(19) United States
(12) Patent Application Publication
(10) Pub. No.: US 2006/0276464 A1
(43) Pub. Date: Dec. 7, 2006

(54) Diarylsulfone sulfonamides and use thereof

(75) Inventors: Ariamala Gopalsamy, Mahwah, NJ (US); William Jay Moore, Collegeville, PA (US); Jeffery Curtis Kern, Gilbertsville, PA (US); Albert John Molinari, Pottstown, PA (US); Mengxiao Shi, New Rochelle, NY (US); Gregory Scott Welmaker, Collegeville, PA (US); Mathew Allan Wilson, Royersford, PA (US); Girija Krishnamurthy, Chestnut Ridge, NY (US); Thomas Joseph Commons, Wayne, PA (US); Michael Byron Webb, Douglassville, PA (US); Richard P. Woodworth, North Wales, PA (US)

Correspondence Address: WOODCOCK WASHBURN LLP, ONE LIBERTY PLACE - 46TH FLOOR, PHILADELPHIA, PA, 19103, US

(73) Assignee: Wyeth, Madison, NJ (US)

(21) Appl. No.: 11/432,788

(22) Filed: May 10, 2006

Related U.S. Application Data
(60) Provisional application No. 60/681,080, filed on May 13, 2005.

(51) Int. Cl. A61K 31/537; A61K 31/538; A61K 31/495; A61K 31/445; A61K 31/4172; A61K 31/40; A61K 31/18

(52) U.S. Cl. 514/230.5; 514/252.12; 514/396; 514/602; 514/237.5; 514/317; 514/352; 544/158; 544/399; 546/229; 564/86; 514/408; 544/92

(57) ABSTRACT

Compounds of Formula 1, or pharmaceutically acceptable salts thereof, are provided: 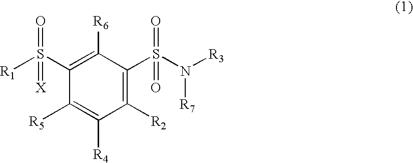 which are modulators of secreted frizzled related protein-1. The compounds, and compositions containing the compounds, can be used to treat a variety of disorders, including osteoporosis.

### CROSS REFERENCE TO RELATED APPLICATIONS

This application claims the benefit of U.S. application Ser. No. 60/681,080, filed May 13, 2005, which is incorporated herein in its entirety.

### FIELD OF THE INVENTION

The present invention relates to novel diarylsulfone sulfonamides that act, for example, as modulators of secreted frizzled-related protein-1. The present invention also relates to processes for the preparation of diarylsulfone sulfonamides and to their use in treating various diseases and disorders.

### BACKGROUND OF THE INVENTION

Bone remodeling, the process by which the adult human skeleton is continuously renewed, is carried out by osteoclasts and osteoblasts, two specialized cell types that originate from hematopoietic and mesenchymal progenitors of the bone marrow, respectively. A continuous and orderly supply of these cells is believed to be essential for skeletal homeostasis, as increased or decreased production of osteoclasts or osteoblasts and/or changes in the rate of their apoptosis are largely responsible for the imbalance between bone resorption and formation that underlies several systemic or localized bone diseases. For example, enhanced osteoclast activity has been found to play a major role in the pathogenesis of postmenopausal osteoporosis, Paget's disease, lytic bone metastases, multiple myeloma, hyperparathyroidism, rheumatoid arthritis, periodontitis, and hypercalcemia of malignancy.

Numerous genes and gene families (and the polypeptides encoded by them) that participate in the regulation of bone cell production and apoptosis have been identified. Wnt proteins have been identified as a family of growth factors consisting of more than a dozen structurally related molecules that are involved in the regulation of fundamental biological processes such as apoptosis, embryogenesis, organogenesis, morphogenesis and tumorigenesis (Nusse and Varmus, Cell 1992, 69:1073-1087). Wnt polypeptides are multipotent factors and have biological activities similar to those of other secretory proteins such as transforming growth factor (TGF)-β, fibroblast growth factors (FGFs), nerve growth factor (NGF), and bone morphogenetic proteins (BMPs).

Studies indicate that certain Wnt proteins interact with a family of proteins named “frizzled” that act as receptors for Wnt proteins or as components of a Wnt receptor complex (in Moon et al., Cell 1997, 88:725-728; Barth et al., Curr. Opin. Cell Biol. 1997, 9:683-690). Frizzled proteins contain an amino terminal signal sequence for secretion, a cysteine-rich domain (CRD) that is thought to bind Wnt, seven putative transmembrane domains that resemble a G-protein coupled receptor, and a cytoplasmic carboxyl terminus. The Frizzled receptors form a signaling complex with another family of membrane receptors known as the low-density lipoprotein (LDL) receptor-related proteins (LRP) (in Logan & Nusse, Annual Review of Cell & Developmental Biology 2004, 20:781-810; Moon et al., Nature Reviews Genetics 2004, 5:691-701).

The first secreted frizzled-related protein (SFRP) was named “Frzb” (for “frizzled motif in bone development”) and was purified and cloned from bovine articular cartilage extracts based on its ability to stimulate in vivo chondrogenic activity in rats (Hoang et al., J. Biol. Chem. 1996, 271:26131-26137; Jones & Jomary, Bioessays 2002, 24:811-820). The human homologue of the bovine gene has also been cloned. Unlike the frizzled proteins, however, Frzb does not contain a serpentine transmembrane domain, and appears to be a secreted receptor for Wnt. The Frzb cDNA encodes a 325 amino acid/36,000 dalton protein and is predominantly expressed in the appendicular skeleton. The highest level of expression is in developing long bones and corresponds to epiphyseal chondroblasts; expression declines and disappeares toward the ossification center.

Studies indicate that SFRPs participate in apoptosis. Some SFRPs have thus been identified as “SARPs” for secreted apoptosis related proteins. Additional members of the SFRP family have been identified, and have been shown to be antagonists of Wnt action. There are currently at least five known human SFRP/SARP genes: SFRP-1/FrzA/FRP-1/SARP-2, SFRP-2/SDF-5/SARP-1, SFRP-3/Frzb-1/FrzB/Fritz, SFRP-4 and SFRP:-5/SARP-3 (Leimeister et al., Mechanisms of Development 1998, 75:2942). Secreted frizzled related protein-1 (SFRP-1) is a Wnt antagonist and is expressed in osteoblasts and osteocytes. Although the precise role that SARPs/SFRPs play in apoptosis is not yet clear, these proteins appear to either suppress or enhance the programmed cell death process. Deletion of SFRP-1 in mice has been shown to lead to decreased osteoblast/osteocyte apoptosis and to increased bone formation. (Bodine, P. V. N, et al., Mol. Endocrinol., 2004, 18(5) 1222-1237.)

A need exists in the art for the identification of modulators of SFRP-1 that can be used as novel agents for the treatment of bone disorders, including bone resorption disorders such as osteoporosis, and for regulation of bone formation in humans.

### SUMMARY OF THE INVENTION

The present invention relates to certain diarylsulfone sulfonamides and to their use, for example, in medical treatment. In one aspect, the invention relates to diarylsulfone sulfonamides that act as modulators of secreted frizzled related protein-1. The compounds can be used, for example, to treat bone disorders such as osteoporosis.

In certain aspects, the present invention is directed to compounds of Formula (1): 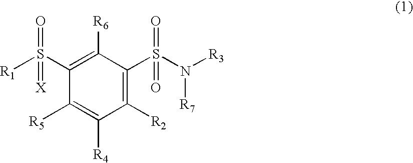
or a pharmaceutically acceptable salt thereof,
wherein

R₁ is 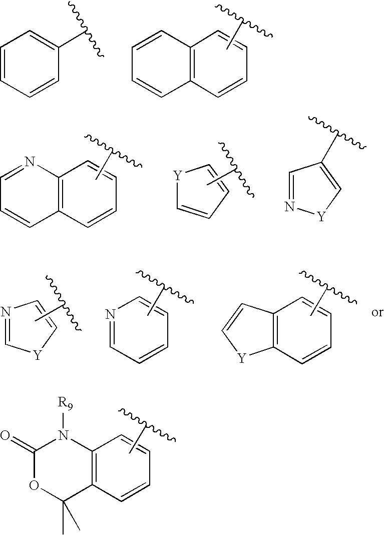
and each R₁ group is optionally substituted with up to three R₈ groups;

Y is O, S, or NR₉;

R₈ is alkyl, arylalkyl, perfluoroalkyl, alkenyl, arylalkenyl, alkynyl, arylalkynyl, cycloalkyl, alkylcycloalkyl, heterocycloalkyl, alkylheterocycloalkyl, aryl, alkylaryl, heteroaryl, alkoxy, perfluoroalkoxy, arylalkoxy, alkylcarbonyl, arylcarbonyl, halogen, cyano, azido, hydroxyl, carboxy, alkoxycarbonyl, alkylamino, dialkylamino, alkylaminocarbonyl, dialkylaminocarbonyl, alkylcarbonylamino, alkylcarbonylalkylamino, hydroxyalkylamino, nitro, alkylcarbonyloxime, alkylsulfonyl, alkylsulfinyl, alkylthio, perfluoroalkylthio, arylthio, tertiary alkylcarbinol, tertiary alkylcycloalkylcarbinol, or tertiary arylalkylcarbinol;

R₉ is hydrogen, alkyl, aryl, arylalkyl, cycloalkylalkyl, heterocycloalkyl, or spirocycloalkyl;

X is oxygen or an electron pair;

R₂ is hydrogen, alkyl, alkoxy, cycloalkyl, perfluoroalkyl, perfluoroalkylalkyl, perfluoroalkoxy, dialkylamino, or halogen;

R₄ is hydrogen, halogen, alkyl, cycloalkyl, alkoxy, perfluoroalkyl, or perfluoroalkoxy;

or R₂ and R₄, together with the carbon atoms to which they are attached, form a cycloalkyl ring of 5 to 7 carbon atoms that is optionally substituted with 1 to 3 R groups;

each R is, independently, hydrogen, alkyl, arylalkyl, cyanoalkyl, cycloalkyl, cycloalkylalkyl, heterocycloalkyl, spirocycloalkyl, aryl, arylalkyl, or alkoxyalkyl;

R₅, and R₆ are, independently, hydrogen, alkyl, aryl, alkoxy, halogen, or perfluoroalkyl;

R₃ and R₇ are each, independently, hydrogen or an optionally substituted alkyl, cycloalkyl, heterocycloalkyl, alkylheterocycloalkyl, heteroarylalkyl, alkylaryl, alkylheteroaryl, alkenyl, alkynyl, fused cycloalkylaryl, fused heterocycloalkylaryl, cycloalkylcarbonyl, or heterocycloalkylcarbonyl group;

or R₃ and R₇, together with the nitrogen atom to which they are attached, form a five or six membered heterocycloalkyl ring optionally substituted with 1 to 5 substituents selected from alkyl, aryl, heterocycloalkyl, heterocycloalkylalkyl, alkylamino, dialkylamino, alkoxycarbonyl, alkylcarbonyl, alkylaminocarbonylalkyl, and heterocycloalkylcarbonylalkyl.

In other embodiments, the invention relates to compositions comprising at least one compound of Formula 1, or a pharmaceutically acceptable salt thereof, and one or more pharmaceutically acceptable excipients, diluents, or carriers.

The present invention also provides methods for treating patients suffering from osteoporosis, arthritis, chronic obstructive pulmonary disease, cartilage defects, bone fractures, or leiomyoma that comprise administering to the patients a therapeutically effective amount of at least one compound of Formula 1.

### DETAILED DESCRIPTION OF ILLUSTRATIVE EMBODIMENTS

The term “alkyl,” as used herein, refers to an optionally substituted aliphatic hydrocarbon chain having 1 to 12 carbon atoms, preferably 1 to 8 carbon atoms, and more preferably 1 to 4 carbon atoms. The term “alkyl” includes straight and branched chains. Straight chain alkyl groups have 1 to 8 carbon atoms and branched chain alkyl groups have 3 to 12 carbon atoms. Examples of alkyl groups include methyl, ethyl, n-propyl, isopropyl, n-butyl, isobutyl, sec-butyl, t-butyl, n-pentyl, isopentyl, neo-pentyl, n-hexyl, and isohexyl groups.

The term “hydroxyalkyl,” as used herein, refers to the group -alkyl-OH where alkyl is an alkyl group as previously defined.

The term “carboxyalkyl,” as used herein, refers to the group -alkyl-C(O)OH where alkyl is an alkyl group as previously defined.

The term “haloalkyl,” as used herein, refers to the group -alkyl-halo where halo is a halogen atom and alkyl is an alkyl group as previously defined.

The term “perfluoroalkyl,” as used herein, refers to an optionally substituted straight or branched aliphatic hydrocarbon chain of 1 to 8 carbon atoms and preferably 1 to 3 carbon atoms, in which all hydrogens are replaced with fluorine.

The term “perfluoroalkylalkyl,” as used herein, refers to the group -alkyl-perfluoroalkyl where alkyl and perfluoroalkyl are as previously defined.

The term “alkenyl,” as used herein, refers to an optionally substituted aliphatic straight or branched hydrocarbon chain having 2 to 12 carbon atoms that contain 1 to 3 double bonds. Straight chain alkenyl groups have 2 to 8 carbon atoms and branched chain alkenyl groups have 3 to 12 carbon atoms. Examples of alkenyl groups include, but are not limited to, vinyl, prop-1-enyl, allyl, but-1-enyl, but-2-enyl, but-3-enyl, 3,3-dimethylbut-1-enyl, or 2-methylvinyl.

The term “alkynyl,” as used herein, refers to an optionally substituted aliphatic straight or branched hydrocarbon chain having 2 to 8 carbon atoms that contains 1 to 3 triple bonds. Straight chain alkynyl groups have 2 to 8 carbon atoms and branched chain alkynyl groups have 5 to 12 carbon atoms.

The term “cycloalkyl,” as used herein, refers to an optionally substituted hydrocarbon ring containing 3 to 12 carbon atoms and preferably 3 to 6 carbon atoms. Cycloalkyl groups may be monocyclic or bicyclic, and may be saturated or partially saturated. The term “bicycloalkyl,” as used herein, refers to a bicyclic cycloalkyl group of 8 to 12 ring carbon atoms. “Bridged” cycloalkyl groups contain at least one carbon-carbon bond between two non-adjacent carbon atoms of the cycloalkyl ring.

The term “alkylcycloalkyl,” as used herein, refers to the group -cycloalkyl-(alkyl)ₙ in which n is 1 to 3, cycloalkyl is a cycloalkyl group as previously defined, and alkyl is an alkyl group as previously defined.

The term “cycloalkylalkyl,” as used herein, refers to the group -alkyl-cycloalkyl in which alkyl is an alkyl group as previously defined and cycloalkyl is a cycloalkyl group as previously defined.

The term “spirocycloalkyl,” as used herein, refers to two optionally substituted cycloalkyl groups as previously defined that are joined by a single sp3 carbon atom that is the only common member of the two joined rings.

The term “heterocycloalkyl,” as used herein, refers to a 3 to 12 membered, and more preferably 5 to 7 membered optionally substituted cycloalkyl group in which one to three carbon atoms of the cycloalkyl group are replaced with a heteroatom independently selected from oxygen, nitrogen, and sulfur, including sulfoxide and sulfonyl. The heterocycloalkyl group may be saturated or partially saturated, and may be monocyclic or bicyclic. The term “heterobicycloalkyl” refers to the bicyclic structure formed when a heterocycloalkyl group is fused to another heterocycloalkyl group, to a cycloalkyl group, to an aryl group, or to a heteroaryl group. Heterobicycloalkyl groups have 8 to 12 ring atoms. “Bridged” heterocycloalkyl groups contain at least one carbon-carbon bond between non-adjacent carbon atoms of the heterocycloalkyl ring.

The term “alkylheterocycloalkyl,” as used herein, refers to the group -heterocycloalkyl-(alkyl)ₙ in which n is 1 to 3, heterocycloalkyl is a heterocycloalkyl group as previously defined, and alkyl is an alkyl group as previously defined.

The term “heterocycloalkylalkyl,” as used herein, refers to the group —R′-heterocycloalkyl where R′ is an alkyl group as previously defined and heterocycloalkyl is a heterocycloalkyl group as previously defined.

The term “aryl,” as used herein refers to an optionally substituted carbocyclic aromatic ring. Aryl groups may be monocyclic or bicyclic. Exemplary aryl groups include phenyl and naphthyl.

The term “carboxyaryl,” as used herein, refers to the group -aryl-C(O)OH, where aryl is an aryl group as previously defined.

The term “heteroaryl,” as used herein refers to an optionally substituted 5 to 10 membered monocyclic or bicyclic carbon containing aromatic ring having 1 to 3 of its ring members independently selected from nitrogen, sulfur and oxygen. Monocyclic rings preferably have 5 to 6 members and bicyclic rings preferably have 8 to 10 membered ring structures. Examples of heteroaryls include, but are not limited to, thienyl, furyl, pyrrolyl, imidazolyl, pyrazolyl, oxazolyl, isoxazolyl, thiazolyl, isothiazolyl, pyridyl, pyrazinyl, pyrimidinyl, pyridazinyl, indolyl, indazolyl, benzofuranyl, isobenzofuranyl, benzothienyl, isobenzothienyl, quinolyl, isoquinolyl, quinoxalinyl, and quinazolinyl.

The term “alkylheteroaryl,” as used herein, refers to the group -heteroaryl-alkyl wherein heteroaryl is a heteroaryl group as previously defined and alkyl is an alkyl group as previously defined.

The term “arylcarbonylalkyl,” as used herein, refers to the group R′-C(O)-aryl where R′ is an alkyl group as previously defined and aryl is an aryl group as previously defined.

The term, “fused cycloalkylaryl,” as used herein, refers to a cycloalkyl group as previously defined fused to an aryl group of five or six carbon atoms as previously defined or fused to a heteroaryl group of five or six atoms as previously defined. The point of attachment can occur at any generally acceptable position.

The term, “fused cycloalkylarylaminocarbonyl,” as used herein, refers to the group —C(O)—NH-fused cycloalkylaryl where fused cycloalkylaryl is a fused cycloalkylaryl group as previously defined.

The term, “fused hetercycloalkylaryl,” as used herein, refers to a heterocycloalkyl group as previously defined fused to an aryl group of five or six carbon atoms as previously defined or fused to a heteroaryl group of five or six atoms as previously defined. The point of attachment can occur at any generally acceptable position.

The term “fused hetercycloalkylarylcarbonyl,” as used herein, refers to the group —C(O)— fused hetercycloalkylaryl where fused hetercycloalkylaryl is a fused hetercycloalkylaryl group as previously defined.

The term “alkylcarbonyl,” as used herein, refers to the group —C(O)R′ where R′ is an alkyl group as previously defined.

The term “alkylthioalkylcarbonyl,” as used herein, refers to the group —C(O)—R′—S—R′ where R′ is an alkyl group as previously defined.

The term “alkylcarbonylamino,” as used herein, refers to the group —NHC(O)R′ where R′ is an alkyl group as previously defined.

The term “alkoxycarbonylamino,” as used herein, refers to the group —NHC(O)OR′ where R′ is an alkyl group as previously defined.

The term “alkylcarbonylalkylamino,” as used herein, refers to the group —NH—R′—C(O)R′ where R′ is an alkyl group as previously defined.

The term “alkylsulfonylamino,” as used herein, refers to the group —NH₂—S(O)₂—R′ where R′ is an alkyl group as previously defined.

The term “carboxyarylsulfonylamino,” as used herein, refers to the group —NH₂—S(O)₂-aryl-C(O)OH where aryl is an aryl group as previously defined.

The term “alkylcarbonyloxime,” as used herein, refers to the group —C(N═OR′)R′ where R′ is an alkyl group as previously defined.

The term “alkoxy,” as used herein, refers to the group —O—R′ where R′ is an alkyl group as previously defined.

The term “perfluoroalkoxy,” as used herein, refers to the group —O—R″ where R″ is a perfluoroalkyl group as previously defined.

The terms “amino,” “alkylamino,” “dialkylamino,” and “imino,” as used herein, refer to the groups —NH₂, —NHR′, —N(R′)₂, and —C═NH, respectively, where each R′ is, independently, an alkyl group as previously defined.

The term “aminoalkyl,” as used herein, refers to the group —R′NH₂ where R′ is an alkyl group as previously defined.

The term “alkylcarbinol,” as used herein, refers to an alkyl group as previously defined substituted with a hydroxyl group.

The term “carboxy,” as used herein, refers to the group —COOH.

The term “carbonyl,” as used herein, refers to a bivalent carbon atom that is further bonded to an oxygen atom through a double bond.

The term “thiocarbonyl,” as used herein, refers to a bivalent carbon atom that is further bonded to a sulfur atom through a double bond.

The terms “halogen” or “halo,” as used herein, refer to chlorine, bromine, fluorine or iodine.

The term “cyano” or “cyanoalkyl,” as used herein, refers to the group —CN or —R′—CN where R′ is an alkyl group as previously defined.

The term “alkoxyalkyl,” as used herein, refers to the group —R′-alkoxy where R′ is an alkyl group as previously defined and alkoxy is an alkoxy group as previously defined.

The term “arylalkyl,” as used herein, refers to the group —R′-aryl where aryl is an aryl group as previously defined, and R′ is an alkyl group as previously defined.

The term “heteroarylalkyl,” as used herein, refers to the group —R′-heteroaryl where heteroaryl is a heteroaryl group as previously defined, and R′ is an alkyl group as previously defined.

The term “arylalkenyl,” as used herein, refers to the group -alkenyl-aryl where aryl is an aryl group as previously defined, and alkenyl is an alkenyl group as previously defined.

The term “arylalkynyl,” as used herein, refers to the group -alkynyl-aryl where aryl is an aryl group as previously defined, and alkynyl is an alkynyl group as previously defined.

The term “arylalkoxy,” as used herein, refers to the group -alkoxy-aryl where aryl is an aryl group as previously defined and alkoxy is an alkoxy group as previously defined. The term “benzoxy” refers to the group —O—CH₂-phenyl.

The term “aminocarbonylalkoxy,” as used herein, refers to the group -alkoxy-C(O)NH₂ where alkoxy is an alkoxy group as previously defined.

The term “alkoxycarbonylalokxy,” as used herein, refers to the group -alkoxy-C(O)-alkoxy where alkoxy is an alkoxy group as previously defined.

The term “carboxyalkoxy,” as used herein, refers to the group -alkoxy-C(O)OH where alkoxy is an alkoxy group as previously defined.

The term “arylalkylcarbonyl,” as used herein, refers to the group -alkylcarbonyl-aryl wherein alkylcarbonyl is an alkylcarbonyl group as previously defined and aryl is an aryl group as previously defined.

The term “arylcarbonyl,” as used herein, refers to the group —C(O)-aryl, where aryl is an aryl group of 6 to 10 carbon atoms as previously defined.

The term “dialkylaminoarylcarbonyl,” as used herein, refers to the group -arylcarbonyl-N(R′)(R′) where arylcarbonyl is an arylcarbonyl group as previously defined.

The term “arylthio,” as used herein, refers to the group —S-aryl where aryl is an aryl group as previously defined.

The term “arylthiol,” as used herein, refers to the group HS-aryl where aryl is an aryl group as previously defined.

The term “arylsulfonyl,” as used herein, refers to the group —S(O)₂-aryl where aryl is an aryl group as previously defined.

The term “arylsulfonylarylsulfonyl,” as used herein, refers to the group —S(O)₂-aryl-S(O)₂-aryl where aryl is an aryl group as previously defined.

The term “carboxyarylsulfonyl,” as used herein, refers to the group —S(O)₂-aryl-C(O)OH where aryl is an aryl group as previously defined.

The term “aminosulfonyl,” as used herein, refers to the group —S(O)₂—NH₂.

The term “heteroarylsulfonyl,” as used herein, refers to the group —S(O)₂-heteroaryl where heteroaryl is a heteroaryl group as previously defined.

The term “arylester,” as used herein, refers to the group —C(O)O-aryl where aryl is an aryl group as previously defined.

The term “alkylcarbonyl,” as used herein, refers to the group —C(O)R′ where R′ is an alkyl group as previously defined.

The term “alkylthiocarbonyl,” as used herein, refers to the group —C(S)R′ where R′ is an alkyl group as previously defined.

The term “alkylaminoalkylcarbonyl,” as used herein, refers to the group —C(O)R′NH(R′) where R′ is an alkyl group as previously defined.

The term “dialkylaminoalkylcarbonyl,” as used herein, refers to the group —C(O)R′N(R′)(R′) where R′ is an alkyl group as previously defined.

The term “perfluoroalkylcarbonyl,” as used herein, refers to the group —C(O)R″ where R″ is a perfluoroalkyl group as previously defined.

The term “carboxyalkylcarbonyl,” as used herein, refers to the group —C(O)R′C(O)OH where R′ is an alkyl group as previously defined.

The term “alkoxycarbonyl,” as used herein, refers to the group —C(O)OR′ where R′ is an alkyl group as previously defined.

The term “alkoxythiocarbonyl,” as used herein, refers to the group —C(S)OR′ where R′ is an alkyl group as previously defined.

The term “alkoxycarbonylalkyl,” as used herein, refers to the group —R′C(O)OR′ where R′ is an alkyl group as previously defined.

The term “arylcarbonyl,” as used herein, refers to the group —C(O)-aryl where aryl is an aryl group as previously defined.

The term “heteroarylcarbonyl,” as used herein, refers to the group —C(O)-heteroaryl where heteroaryl is a heteroaryl group as previously defined.

The term “heteroarylalkylcarbonyl,” as used herein, refers to the group —C(O)—R′-heteroaryl where heteroaryl is a heteroaryl group as previously defined and R′ is an alkyl group as previously defined.

The term “heterocycloalkylalkylcarbonyl,” as used herein, refers to the group —C(O)—R′-heterocycloalkyl where heterocycloalkyl is a heterocycloalkyl group as previously defined and R′ is an alkyl group as previously defined.

The term “heterocycloalkylalkylaminothiocarbonyl,” as used herein, refers to the group —C(O)—S—NH—R′-heterocycloalkyl where heterocycloalkyl is a heterocycloalkyl group as previously defined and R′ is an alkyl group as previously defined.

The term “aryloxycarbonyl,” as used herein, refers to the group —C(O)—O-aryl where aryl is an aryl group as previously defined.

The term “aryloxythiocarbonyl,” as used herein, refers to the group —C(S)—O-aryl where aryl is an aryl group as previously defined.

The term “cyanoarylcarbonyl,” as used herein, refers to the group —C(O)-aryl-CN where aryl is an aryl group as previously defined.

The term “arylalkylcarbonyl,” as used herein, refers to the group —C(O)—R′-aryl where R′ is an alkyl group as previously defined and aryl is an aryl group as previously defined.

The term “cycloalkylcarbonyl,” as used herein, refers to the group —C(O)-cycloalkyl where cycloalkyl is a cycloalkyl group as previously defined.

The term “heterocycloalkylcarbonyl,” as used herein, refers to the group —C(O)-heterocycloalkyl where heterocycloalkyl is a heterocycloalkyl group as previously defined.

The term “heterocycloalkylthiocarbonyl,” as used herein, refers to the group —C(S)-heterocycloalkyl where heterocycloalkyl is a heterocycloalkyl group as previously defined.

The term “aminoalkylcarbonyl,” as used herein, refers to the group —C(O)—R′—NH₂ where R′ is an alkyl group as previously defined.

The term “alkoxycarbonylaminothiocarbonyl,” as used herein, refers to the group —C(O)—S—NH—C(O)—O—R′ where R′ is an alkyl group as previously defined.

The term “alkoxycarbonylalkylaminothiocarbonyl,” as used herein, refers to the group —C(O)—S—NH—R′—C(O)—O—R′ where R′ is an alkyl group as previously defined.

The term “alkylthiocarbonylalkylcarbonyl,” as used herein, refers to the group —C(O)—R′—C(O)—S—R′ where R′ is an alkyl group as previously defined.

The term “cyanoalkoxycarbonyl,” as used herein, refers to the group —C(O)-alkoxy-CN where alkoxy refers to an alkoxy group as previously defined.

The term “alkylaryl,” as used herein, refers to the group -aryl-R′ where R′ is an alkyl group as previously defined, and aryl is an aryl group as previously defined.

The term “alkylester,” as used herein, refers to the group —C(O)OR′ wherein R′ is an alkyl group as previously defined.

The term “aminocarbonyl,” as used herein, refers to the group —C(O)NH₂.

The terms “alkylaminocarbonyl,” and “dialkylaminocarbonyl,” as used herein, refer to the groups —C(O)NHR′ and —C(O)N(R′)₂, respectively, where each R′ is, independently, an alkyl group as previously defined.

The term “heterocycloalkylaminocarbonyl,” as used herein, refers to the group —C(O)NH-heterocycloalkyl where heterocycloalkyl is a heterocycloalkyl group as previously defined.

The term “carboxyalkylcarbonylheterocycloalkylaminocarbonyl,” as used herein, refers to the group -heterocycloalkylaminocarbonyl-C(O)—R′—C(O)OH where heterocycloalkylaminocarbonyl is a heterocycloalkylaminocarbonyl group as previously defined and R′ is an alkyl group as previously defined.

The term “carboxyalkylaminocarbonyl,” as used herein, refers to the group -alkylaminocarbonyl-carboxy where carboxy is a carboxy group as previously defined and alkylaminocarbonyl is an alkylaminocarbonyl group as previously defined.

The term “alkoxycarbonylalkylaminocarbonyl,” as used herein, refers to the group -alkylaminocarbonyl-carbonyl-alkoxy where alkoxy is an alkoxy group as previously defined, carbonyl is a carbonyl group as previously defined, and alkylaminocarbonyl is an alkylaminocarbonyl group as previously defined.

The term “aminocarbonylalkyl,” as used herein, refers to the group —R′C(O)NH₂ where R′ is an alkyl group as previously defined.

The terms “alkylaminocarbonylalkyl,” and “dialkylaminocarbonylalkyl,” as used herein, refer to the groups —R′C(O)NHR′ and —R′C(O)N(R′)₂, respectively, where each R′ is, independently, an alkyl group as previously defined.

The terms “alkylaminothiocarbonyl,” and “dialkylaminothiocarbonyl,” as used herein, refer to the groups —C(S)NHR′ and —C(S)N(R′)₂, respectively, where each R′ is, independently, an alkyl group as previously defined.

The term “heterocycloalkylcarbonylalkyl,” as used herein, refers to the group —R′C(O)heterocycloalkyl where R′ is an alkyl group as previously defined and heterocycloalkyl is a heterocycloalkyl group as previously defined.

The term “arylaminocarbonyl,” as used herein, refers to the group —C(O)NH(aryl), where aryl is an aryl group as previously defined.

The term “heteroarylaminocarbonyl,” as used herein, refers to the group —C(O)NH(heteroaryl), where heteroaryl is a heteroaryl group as previously defined.

The term “heteroarylaminothiocarbonyl,” as used herein, refers to the group —C(S)NH(heteroaryl), where heteroaryl is a heteroaryl group as previously defined.

The term “arylaminothiocarbonyl,” as used herein, refers to the group —C(S)NH(aryl), where aryl is an aryl group as previously defined.

The term “cycloalkylaminocarbonyl,” as used herein, refers to an alkylaminocarbonyl or dialkylaminocarbonyl group as previously defined in which at least one alkyl group is replaced by a cycloalkyl group.

The term “alkylsulfonyl,” as used herein, refers to the group —S(O)₂—R′ where R′ is an alkyl group as previously defined.

The term “alkylsulfinyl,” as used herein, refers to the group —S(O)—R′ where R′ is an alkyl group as previously defined.

The term “alkylthio,” as used herein, refers to the group —S—R′ where R′ is an alkyl group as previously defined.

The term “perfluoroalkylthio,” as used herein, refers to the group —S—R″ where R″ is a perfluoroalkyl group as previously defined.

The term “tertiary alkylcarbinol,” as used herein, refers to the group —C(R′)₂OH where R′ is an alkyl group as previously defined.

The term “tertiary cycloalkylcarbinol,” as used herein, refers to the group —C(cycloalkyl)₂OH where cycloalkyl refers to a cycloalkyl group as previously defined.

The term “tertiary alkylcycloalkylcarbinol,” as used herein, refers to the group —C(R′)(cycloalkyl)OH where R′ refers to an alkyl group as previously defined, and cycloalkyl refers to a cycloalkyl group as previously defined.

The term “tertiary arylcarbinol,” as used herein, refers to the group —C(aryl)₂OH where each “aryl” independently refers to an aryl group as previously defined.

The term “tertiary arylalkylcarbinol,” as used herein, refers to the group —C(R′)(aryl)OH where R′ is an alkyl group as previously defined, and aryl is an aryl group as previously defined.

The term “phosphonic acid alkyl,” as used herein, refers to the group —R′—P(O)(OH)₂ where R′ is an alkyl group as previously defined.

The term “dimethylphosphonatealkyl,” as used herein, refers to the group —R′—P(O)(OCH₃)₂ where R′ is an alkyl group as previously defined.

The term “partially saturated,” as used herein, refers to a nonaromatic cycloalkyl or heterocycloalkyl group containing at least one double bond and preferably one or two double bonds.

The term “therapeutically effective amount,” as used herein, refers to the amount of a compound of formula 1 that, when administered to a patient, is effective to at least partially treat a condition from which the patient is suffering or is suspected to suffer. Such conditions include, but are not limited to, osteoporosis, arthritis, chronic obstructive pulmonary disease, cartilage defects, bone fractures, and leiomyoma.

The term “pharmaceutically acceptable salts” or “pharmaceutically acceptable salt” includes acid addition salts, namely salts derived from treating a compound of formula 1 with an organic or inorganic acids or bases. Where the compound having formula I has an acidic function, for instance where R₃ is carboxyalkyl or R₈ is carboxy or phenolic hydroxyl, the term “pharmaceutically acceptable salts” or “pharmaceutically acceptable salt” includes salts derived from bases, for instance, sodium salts.

The term “patient,” as used herein, refers to a mammal.

The terms “administer,” “administering,” or “administration,” as used herein, refer to either directly administering a compound or composition to a patient, or administering a prodrug derivative or analog of the compound to the patient, which will form an equivalent amount of the active compound or substance within the patient's body.

The terms “treat” and “treating,” as used herein, refer to partially or completely alleviating, inhibiting, preventing, ameliorating and/or relieving a condition from which a patient is suspected to suffer.

The terms “suffer” and “suffering,” as used herein, refer to one or more conditions with which a patient has been diagnosed, or is suspected to have.

Certain embodiments of the invention relate to compounds of Formula (1): 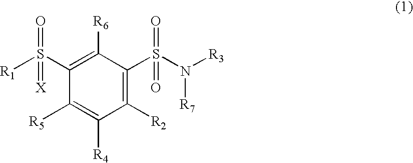
or a pharmaceutically acceptable salt thereof,
wherein

R₁ is 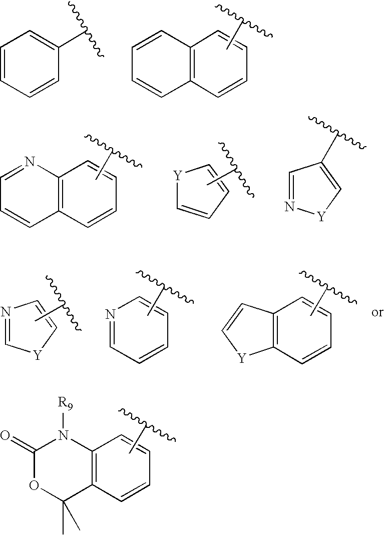
and each R₁ group is optionally substituted with up to three R₈ groups;

Y is O, S, or NR₉;

R₈ is alkyl, arylalkyl, perfluoroalkyl, alkenyl, arylalkenyl, alkynyl, arylalkynyl, cycloalkyl, alkylcycloalkyl, heterocycloalkyl, alkylheterocycloalkyl, aryl, alkylaryl, heteroaryl, alkoxy, perfluoroalkoxy, arylalkoxy, alkylcarbonyl, arylcarbonyl, halogen, cyano, azido, hydroxyl, carboxy, alkoxycarbonyl, alkylamino, dialkylamino, alkylaminocarbonyl, dialkylaminocarbonyl, alkylcarbonylamino, alkylcarbonylalkylamino, hydroxyalkylamino, nitro, alkylcarbonyloxime, alkylsulfonyl, alkylsulfinyl, alkylthio, perfluoroalkylthio, arylthio, tertiary alkylcarbinol, tertiary alkylcycloalkylcarbinol, or tertiary arylalkylcarbinol;

R₉ is hydrogen, alkyl, aryl, arylalkyl, cycloalkylalkyl, heterocycloalkyl, or spirocycloalkyl;

X is oxygen or an electron pair;

R₂ is hydrogen, alkyl, alkoxy, cycloalkyl, perfluoroalkyl, perfluoroalkylalkyl, perfluoroalkoxy, dialkylamino, or halogen;

R₄ is hydrogen, halogen, alkyl, cycloalkyl, alkoxy, perfluoroalkyl, or perfluoroalkoxy;

or R₂ and R₄, together with the carbon atoms to which they are attached, form a cycloalkyl ring of 5 to 7 carbon atoms that is optionally substituted with 1 to 3 R groups;

each R is, independently, hydrogen, alkyl, arylalkyl, cyanoalkyl, cycloalkyl, cycloalkylalkyl, heterocycloalkyl, spirocycloalkyl, aryl, arylalkyl, or alkoxyalkyl;

R₅, and R₆ are, independently, hydrogen, alkyl, aryl, alkoxy, halogen, or perfluoroalkyl;

R₃ and R₇ are each, independently, hydrogen or an optionally substituted alkyl, cycloalkyl, heterocycloalkyl, alkylheterocycloalkyl, heteroarylalkyl, alkylaryl, alkylheteroaryl, alkenyl, alkynyl, fused cycloalkylaryl, fused heterocycloalkylaryl, cycloalkylcarbonyl, or heterocycloalkylcarbonyl group;

or R₃ and R₇, together with the nitrogen atom to which they are attached, form a five or six membered heterocycloalkyl ring optionally substituted with 1 to 5 substituents selected from alkyl, aryl, heterocycloalkyl, heterocycloalkylalkyl, alkylamino, dialkylamino, alkoxycarbonyl, alkylcarbonyl, alkylaminocarbonylalkyl, and heterocycloalkylcarbonylalkyl;

provided that the compound is not

2-methyl-N-(2-phenylethyl)-5-(phenylsulfonyl)-benzenesulfonamide;

2-methyl-N-(2-phenylmethyl)-5-(phenylsulfonyl)-benzenesulfonamide;

5-[(4-chlorophenyl)sulfonyl]-N-cyclohexyl-2-methylbenzenesulfonamide;

N-benzyl-5-[(4-chlorophenyl)sulfonyl]-2-methylbenzenesulfonamide;

5-[(4-chlorophenyl)sulfonyl]-N-(2-furylmethyl)-2-methylbenzenesulfonamide;

5-[(4-chlorophenyl)sulfonyl]-2-methylbenzenesulfonamide;

2-methyl-5-(phenylsulfonyl)-benzenesulfonamide;

5-[(4-bromophenyl)sulfonyl]-2-methylbenzenesulfonamide;

2-methyl-5-[(4-nitrophenyl)sulfonyl]-benzenesulfonamide;

5-[(2,4-dinitrophenyl)sulfonyl]-2-methyl-benzenesulfonamide; or

5-[(4-chlorophenyl)sulfonyl]-2-methyl-N-(3-pyridinylmethyl)-benzenesulfonamide.

Any carbon or nitrogen atom in R₁, R₂, R₃, R₄, R₅, R₆, and R₇, as defined herein, may be optionally substituted with one or more substituents understood by those skilled in the art to be suitable substituents. The following lists provide examples of such substituents.

The alkyl, cycloalkyl, alkylheterocycloalkyl, heteroarylalkyl, alkylaryl, alkylheteroaryl, alkenyl, alkynyl, fused cycloalkylaryl, fused heterocycloalkylaryl, cycloalkylcarbonyl, and heterocycloalkylcarbonyl groups of R₃ and R₇ may each be, independently, optionally substituted with 1 to 5 substituents selected from alkyl, perfluoroalkyl, cycloalkyl, heterocycloalkyl, aryl, heteroaryl, fused cycloalkylaryl, alkoxy, aminocarbonylalkoxy, alkoxycarbonylalkoxy, carboxyalkoxy, cycloalkyloxy, aryloxy, amino, alkylamino, dialkylamino, alkoxycarbonylamino, carboxy, cyano, halogen, oxo, hydroxyl, alkylcarbonyl, carboxyalkylcarbonyl, arylaminocarbonyl, heterocycloalkylcarbonyl, alkoxycarbonyl, alkylaminocarbonyl, dialkylaminocarbonyl, fused cycloalkylarylaminocarbonyl, and fused heterocycloalkylarylcarbonyl.

The cycloalkyl, heterocycloalkyl, aryl, and heteroaryl substituents on the alkyl groups of R₃ and R₇ may be, independently, optionally substituted with 1 to 5 substituents selected from alkyl, cycloalkyl, heterocycloalkyl, spirocycloalkyl, perfluoroalkyl, haloalkyl, cyanoalkyl, carboxyalkyl, dimethylphosphonatealkyl, phosphonic acid alkyl, arylalkyl, cycloalkylalkyl, alkoxy, perfluoroalkoxy, arylalkoxy, benzoxy, aryl, heteroaryl, carboxyaryl, arylcarbonyl, alkylcarbonyl, perfluoroalkylcarbonyl, alkoxycarbonyl, carboxyalkylcarbonyl, aryloxycarbonyl, alkoxythiocarbonyl, aryloxythiocarbonyl, arylcarbonyl, cycloalkylcarbonyl, heterocycloalkylcarbonyl, heterocycloalkylthiocarbonyl, arylthiocarbonyl, alkylaminocarbonyl, dialkylaminocarbonyl, dialkylaminoarylcarbonyl, dialkylaminoalkylcarbonyl, alkylthiocarbonyl, arylaminocarbonyl, heteroarylcarbonyl, heteroarylaminocarbonyl, alkylaminothiocarbonyl, dialkylaminothiocarbonyl, arylaminothiocarbonyl, heteroarylaminothiocarbonyl, aminosulfonyl, alkylsulfonyl, arylsulfonyl, arylsulfonylarylsulfonyl, carboxyarylsulfonyl, nitro, amino, dialkylamino, alkylcarbonylamino, alkylsulfonylamino, carboxyarylsulfonylamino, hydroxy, carboxy, sulfonamide, alkylthio, halogen, cyano, guanidine, and oxo, and the nitrogen atoms in the heteroaryl substituents may be optionally substituted with an oxygen atom.

The heterocycloalkyl groups of R₃ and R₇ may be independently, optionally substituted with 1 to 5 substituents selected from alkyl, hydroxyalkyl, cyanoalkyl, carboxyalkyl, aminocarbonylalkyl, alkoxycarbonylalkyl, aminocarbonylalkyl, alkylaminocarbonylalkyl, dialkylaminocarbonylalkyl, heterocycloalkyl, heterocycloalkylalkyl, heterocycloalkylcarbonylalkyl, arylalkyl, heteroarylalkyl, arylcarbonylalkyl, alkylcarbonyl, cyano, alkylester, alkylamide, cycloalkylamide, aryl, arylester, alkylcarbonyl, perfluoroalkylcarbonyl, aminocarbonyl, arylaminocarbonyl, arylaminothiocarbonyl, cyanoalkoxycarbonyl, cycloalkylcarbonyl, arylcarbonyl, arylthiocarbonyl, alkylaminocarbonyl, dialkylaminocarbonyl, arylaminocarbonyl, alkylaminothiocarbonyl, dialkylaminothiocarbonyl, heteroarylcarbonyl, heterocycloalkylcarbonyl, cyanoarylcarbonyl, arylalkylcarbonyl, alkoxycarbonyl, alkoxyalkylcarbonyl, alkylthioalkylcarbonyl, alkylaminoalkylcarbonyl, dialkylaminoalkylcarbonyl, heterocycloalkylalkylcarbonyl, heterocycloalkylalkylaminothiocarbonyl, arylaminothiocarbonyl, heteroarylaminothiocarbonyl, heteroarylalkylcarbonyl, carboxyalkylcarbonyl, alkoxycarbonylaminothiocarbonyl, alkoxycarbonylalkylaminothiocarbonyl, alkylthiocarbonylalkylcarbonyl, alkylsulfonyl, arylsulfonyl, alkylaminoarylsulfonyl, and heteroarylsulfonyl.

The cycloalkyl, heterocycloalkyl, aryl, and heteroaryl substituents on the heterocycloalkyl groups of R₃ and R₇ may be independently, optionally substituted with 1 to 5 substituents selected from alkyl, cycloalkyl, heterocycloalkyl, spirocycloalkyl, perfluoroalkyl, haloalkyl, cyanoalkyl, carboxyalkyl, dimethylphosphonatealkyl, phosphonic acid alkyl, arylalkyl, cycloalkylalkyl, alkoxy, perfluoroalkoxy, arylalkoxy, benzoxy, aryl, heteroaryl, carboxyaryl, arylcarbonyl, alkylcarbonyl, perfluoroalkylcarbonyl, alkoxycarbonyl, carboxyalkylcarbonyl, aryloxycarbonyl, alkoxythiocarbonyl, aryloxythiocarbonyl, arylcarbonyl, cycloalkylcarbonyl, heterocycloalkylcarbonyl, heterocycloalkylthiocarbonyl, arylthiocarbonyl, alkylaminocarbonyl, dialkylaminocarbonyl, dialkylaminoarylcarbonyl, dialkylaminoalkylcarbonyl, alkylthiocarbonyl, arylaminocarbonyl, heteroarylcarbonyl, heteroarylaminocarbonyl, alkylaminothiocarbonyl, dialkylaminothiocarbonyl, arylaminothiocarbonyl, heteroarylaminothiocarbonyl, aminosulfonyl, alkylsulfonyl, arylsulfonyl, arylsulfonylarylsulfonyl, carboxyarylsulfonyl, nitro, amino, dialkylamino, alkylcarbonylamino, alkylsulfonylamino, carboxyarylsulfonylamino, hydroxy, carboxy, sulfonamide, alkylthio, halogen, cyano, guanidine, and oxo, and the nitrogen atoms in the heteroaryl substituents may be optionally substituted with an oxygen atom.

The amino substituents on the alkyl groups of R₃ and R₇ may be, independently, optionally substituted with 1 or 2 substituents selected from alkyl, hydroxyalkyl, carboxyalkyl, cycloalkyl, alkoxycarbonylalkyl, aryl, alkylcarbonyl, arylcarbonyl, alkylsulfonyl, arylsulfonyl, alkylcarbonyl, cycloalkylcarbonyl, alkylaminocarbonyl, dialkylaminocarbonyl, alkylaminothiocarbonyl, dialkylaminothiocarbonyl, alkoxycarbonylalkylaminocarbonyl, carboxyalkylcarbonyl, carboxyalkylaminocarbonyl, carboxyalkylcarbonylheterocycloalkylaminocarbonyl, arylaminocarbonyl, arylcarbonyl, heteroarylaminocarbonyl, heterocycloalkylcarbonyl, arylaminothiocarbonyl, heteroarylaminothiocarbonyl, heterocycloalkylaminocarbonyl, heterocycloalkylthiocarbonyl, heteroarylcarbonyl, alkoxycarbonyl, aryloxycarbonyl, alkoxythiocarbonyl, and aryloxythiocarbonyl.

The cycloalkyl, heterocycloalkyl, aryl, and heteroaryl substituents on the amino substituents on the alkyl groups of R₃ and R₇ may be, independently, optionally substituted with 1 to 5 substituents selected from alkyl, cycloalkyl, heterocycloalkyl, spirocycloalkyl, perfluoroalkyl, haloalkyl, cyanoalkyl, carboxyalkyl, dimethylphosphonatealkyl, phosphonic acid alkyl, arylalkyl, cycloalkylalkyl, alkoxy, perfluoroalkoxy, arylalkoxy, benzoxy, aryl, heteroaryl, carboxyaryl, arylcarbonyl, alkylcarbonyl, perfluoroalkylcarbonyl, alkoxycarbonyl, carboxyalkylcarbonyl, aryloxycarbonyl, alkoxythiocarbonyl, aryloxythiocarbonyl, arylcarbonyl, cycloalkylcarbonyl, heterocycloalkylcarbonyl, heterocycloalkylthiocarbonyl, arylthiocarbonyl, alkylaminocarbonyl, dialkylaminocarbonyl, dialkylaminoarylcarbonyl, dialkylaminoalkylcarbonyl, alkylthiocarbonyl, arylaminocarbonyl, heteroarylcarbonyl, heteroarylaminocarbonyl, alkylaminothiocarbonyl, dialkylaminothiocarbonyl, arylaminothiocarbonyl, heteroarylaminothiocarbonyl, aminosulfonyl, alkylsulfonyl, arylsulfonyl, arylsulfonylarylsulfonyl, carboxyarylsulfonyl, nitro, amino, dialkylamino, alkylcarbonylamino, alkylsulfonylamino, carboxyarylsulfonylamino, hydroxy, carboxy, sulfonamide, alkylthio, halogen, cyano, guanidine, and oxo, and the nitrogen atoms in the heteroaryl substituents may be optionally substituted with an oxygen atom.

The alkyl group substituents on the heterocycloalkyl ring formed from R₃ and R₇, together with the nitrogen atom to which they are attached may each be, independently, optionally substituted with 1 to 5 substituents selected from aryl, heteroaryl optionally substituted with one to three alkyl groups, aminoalkyl, heterocycloalkyl, fused heterocycloalkylaryl, and heterocycloalkylcarbonyl.

In preferred embodiments of the invention R₁ of Formula 1 is aryl.

In other embodiments of the invention, R₁ of Formula 1 is

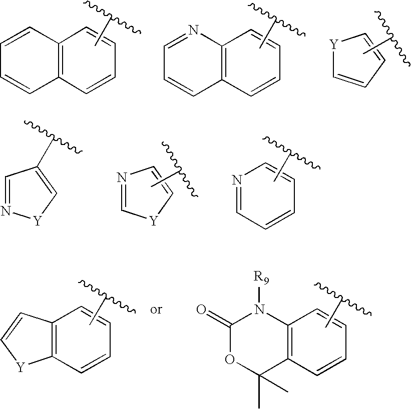

Certain aspects of the invention relate to compounds of Formula 1 wherein each R₈ is, independently, alkyl, alkylaryl, alkylheteroaryl, alkylamino, dialkylamino, carboxy, alkylcarbonyl, alkoxy, perfluoroalkoxy, halogen, or cyano.

Other aspects of the invention are directed to compounds of Formula 1 in which X is oxygen.

Additional embodiments of the invention relate to compounds of Formula 1 in which R₄, R₅, and R₆ are each hydrogen. Alternative embodiments of the invention relate to compounds of Formula 1 in which R₄ is methyl and R₅ and R₆ are each hydrogen, or R₅ is methyl and R₄ and R₆ are each hydrogen, or R₆ is methyl and R₄ and R₅ are each hydrogen.

Further embodiments of the invention are directed to compounds of Formula I in which R₄ is halogen, alkyl, cycloalkyl, alkoxy, perfluoroalkyl, or perfluoroalkoxy.

Other aspects of the invention are directed to compounds of Formula I in which R₅, and R₆ are, independently, alkyl, alkoxy, halogen, or perfluoroalkyl.

Certain embodiments of the invention are directed to compounds of Formula 1 in which R₂ is methyl, ethyl, isopropyl, propyl, Cl, methoxy, trifluoromethyl, or trifluoromethoxy. In preferred embodiments, R₂ is methyl, isopropyl, trifluoromethyl, or trifluoromethoxy. In particularly preferred embodiments, R₂ is isopropyl or trifluoromethyl.

Still further embodiments of the invention relate to compounds of Formula I in which R₂ is hydrogen, alkoxy, cycloalkyl, perfluoroalkyl, perfluoroalkylalkyl , perfluoroalkoxy, dialkylamino, or halogen.

Other aspects of the invention relate to compounds of Formula 1 in which R₃ and R₇, together with the nitrogen atoms to which they are attached, form an optionally substituted 5 or 6 membered heterocycloalkyl group. In preferred aspects, R₃ and R₇, together with the nitrogen atoms to which they are attached, form an optionally substituted piperazinyl group.

Additional embodiments of the invention relate to compounds of Formula I in which R₃ and R₇ are each, independently, alkyl, heterocycloalkyl, heterocycloalkylalkyl, alkylheterocycloalkyl, arylalkyl, heteroarylalkyl, alkenyl, alkynyl, fused cycloalkylaryl, fused heterocycloalkylaryl, cycloalkylcarbonyl, alkoxyalkyl, alkoxycarbonylalkyl, cyanoalkyl, aminoalkyl, dialkylaminocarbonylalkyl, or alkylalkylaminocarbonylalkyl.

Further embodiments of the invention are directed to compounds of Formula 1 in which R₇ is hydrogen and R₃ is alkyl, cycloalkyl, 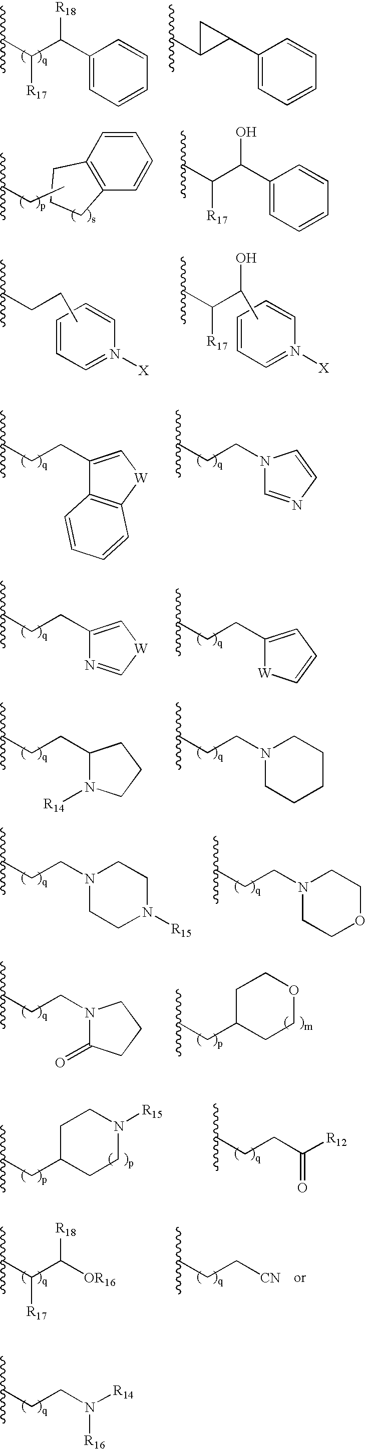
wherein the carbon atoms of each alkyl, cycloalkyl, heterocycloalkyl, aryl or heteroaryl group are optionally substituted with up to four R₁₃ groups;

R₁₃ is hydrogen, F, Cl, Br, alkyl, alkoxy, aryl, nitro, aminosulfonyl, arylalkoxy, perfluoroalkyl, perfluoroalkoxy, amino, alkylamino, dialkylamino, hydroxy, carboxy, cycloalkyl, carboxyalkyl, carboxyalkoxy, alkoxycarbonyl, aminocarbonylalkyl, alkoxycarbonylalkoxy,aminocarbonylalkoxy, alkylaminocarbonylalkyl, aminocarbonyl, alkylamninocarbonyl, dialkylaminocarbonyl, heterocycloalkylcarbonyl, heterocycloalkylaminocarbonyl, alkylaminocarbonylalkoxy, dialkylaminocarbonylalkyl, dialkylaminocarbonylalkoxy, heterocycloalkylcarbonylalkyl, heterocycloalkylaminocarbonylalkoxy, heterocycloalkylcarbonylalkoxy, cycloalkylaminoalkyl, heterocycloalkylaminoalkyl, aminoaaminominoalkylarylamnionalkyl, heteroarylaminoalkylarylalkylaminoalkyl, heteroarylalkylaminoalkyl, alkylaminoalkylamino, dialkylaminoalkylaminoarylamino, heteroarylaminoarylalkylamino, heteroarylalkylamino, or cyano;

each R₁₂ is alkyl, aryl, alkylamino, dialkylamino, alkoxy, hydroxyl, amino, arylamino, diarylamino, aryl(alkyl)amino, or aryloxy;

each R₁₄ is hydrogen, alkyl, aryl, cycloalkyl, alkylcarbonyl, arylcarbonyl, aryloxycarbonyl, alkylsulfonyl, arylsulfonyl, alkylaminocarbonyl, dialkylaminocarbonyl, alkylaminothiocarbonyl, dialkylaminothiocarbonyl, arylaminocarbonyl, heteroarylaminocarbonyl, heterocycloalkylcarbonyl, arylaminothiocarbonyl, heteroarylaminothiocarbonyl, heterocycloalkylthiocarbonyl, heteroarylcarbonyl, alkoxycarbonyl, aryloxycarbonyl, alkoxythiocarbonyl, alkoxycarbonylaminothiocarbonyl, cycloalkylcarbonyl, aminocarbonyl, alkoxycarbonyl, cycloalkylaminocarbonyl, heterocycloalkylaminocarbonyl, cycloalkylsulfonyl, heterocycloalkylsulfonyl, heteroarylsulfonyl, or aryloxythiocarbonyl;

R₁₅ is hydrogen, alkyl, aryl, cycloalkyl, alkylcarbonyl, arylcarbonyl, alkylsulfonyl, arylsulfonyl, alkylcarbonyl, arylcarbonyl, alkylaminocarbonyl, dialkylaminocarbonyl, alkylaminothiocarbonyl, dialkylaminothiocarbonyl, arylaminocarbonyl, heteroarylaminocarbonyl, heterocycloalkylcarbonyl, arylaminothiocarbonyl, heteroarylaminothiocarbonyl, heterocycloalkylthiocarbonyl, heterocycloalkylalkylaminothiocarbonyl, heteroarylcarbonyl, alkoxycarbonyl, aryloxycarbonyl, arylthiocarbonyl, alkoxythiocarbonyl, or aryloxythiocarbonyl;

R₁₆, R₁₇ and R₁₈ are each, independently, hydrogen, alkyl, aryl or cycloalkyl;

m is 0, 1, or 2;

p is 0, 1, or 2;

q is 1 or 2;

s is 1 or 2; and

W is NR₉, O, or S.

The alkyl, aryl and cycloalkyl groups of R₁₃, R₁₄ and R₁₅ may each be, independently, optionally substituted with 1 to 5 substituents selected from alkyl, cycloalkyl, heterocycloalkyl, perfluoroalkyl, aryl, heteroaryl, alkoxy, aryloxy, cycloalkyloxy, amino, alkylamino, dialkylamino, carboxy, cyano, oxo, hydroxyl, alkylcarbonyl, alkoxycarbonyl, arylcarbonyl, alkoxycarbonylamino, alkylcarbonylamino, aminocarbonyl, alkylaminocarbonyl, dialkylaminocarbonyl, alkylthio, alkyloxothio, and alkoxycarbonylalkylamino.

The arylsulfonyl, arylcarbonyl and heteroarylcarbonyl groups of R₁₃, R₁₄ and R₁₅ may be each, independently, optionally substituted with 1 to 5 substituents selected from hydrogen, halogen, hydroxyl, alkyl, cycloalkyl, perfluoroalkyl, aryl, alkoxy, heterocycloalkyl, heteroaryl, cycloalkyloxy, aryloxy, amino, alkylamino, dialkylamino, alkylthio, alkyloxothio, carboxy, cyano, oxo, alkylcarbonyl, arylcarbonyl,alkoxycarbonyl, alkylcarbonylamino, aminocarbonyl, alkylaminocarbonyl, and dialkylaminocarbonyl.

The heterocycloalkyl groups of R₁₃, R₁₄ and R₁₅ may each be, independently, optionally substituted with 1 to 5 substituents selected from hydrogen, hydroxyl, alkyl, cycloalkyl, perfluoroalkyl, aryl, heterocycloalkyl, heteroaryl, heteroarylcarbonyl, heteroarylalkylcarbonyl, alkylcarbonyl, alkylsulfonyl, arylsulfonyl, alkoxycarbonyl, arylcarbonyl, heteroarylcarbonyl, alkylaminocarbonyl, dialkylaminocarbonyl, alkylaminoalkylcarbonyl, dialkylaminoalkylcarbonyl, alkylaminocarbonylalkyl, dialkylaminocarbonylalkyl, heterocycloalkylcarbonylalkyl, carboxyalkylcarbonyl, and arylaminocarbonyl.

In addition, the alkylcarbonyl groups of R₁₃, R₁₄ and R₁₅ may each be, independently, optionally substituted with 1 to 5 substituents selected from amino, alkylamino, dialkylamino, cycloalkyl, heterocycloalkyl, perfluoroalkyl, aryl, heteroaryl, alkoxy, aryloxy, cycloalkyloxy, carboxy, cyano, oxo, hydroxyl, alkylcarbonyl, alkoxycarbonyl, arylcarbonyl, and alkoxycarbonylamino.

The amino groups of the alkylamino and hetercycloalkylamino groups of R₁₃, R₁₄ and R₁₅ may each be, independently, optionally substituted with a substituent selected from hydrogen, alkyl, cycloalkyl, and aryl.

Particular embodiments of the invention relate to compounds of Formula 1 in which R₇ is hydrogen and R₃ is heteroarylethyl, heteroarylpropyl, arylethyl, heterocycloalkyl, heterocycloalkylethyl, heterocycloalkylpropyl, heterocycloalkylmethyl, heterocyclalkylamino, cycloalkyl, fused cycloalkylaryl, aminoalkyl, or alkoxyalkyl. In preferred embodiments R₇ is hydrogen and R₃ is heteroarylethyl, heteroarylpropyl, heterocyclalkylamino, fused cycloalkylaryl, or phenylethyl. In particularly preferred embodiments R₇ is hydrogen and R₃ is pyridinylethyl, imidazolylethyl, imidazolylpropyl, heterocyclalkylamino, fused cycloalkylaryl, or phenylethyl.

Specific, representative compounds of Formula 1 include:
N-(2-phenylethyl)-3-(phenylsulfonyl)benzenesulfonamide;
N-[2-(2-chlorophenyl)ethyl]-3-(phenylsulfonyl)benzenesulfonamide;
N-[2-(2-methoxyphenyl)ethyl]-3-(phenylsulfonyl)benzenesulfonamide;
N-[2-(3-methoxyphenyl)ethyl]-3-(phenylsulfonyl)benzenesulfonamide;
N-[2-(3,4-dimethoxyphenyl)ethyl]-3-(phenylsulfonyl)benzenesulfonamide;
N-[2-(4-bromophenyl)ethyl]-3-(phenylsulfonyl)benzenesulfonamide;
N-[2-(4-fluorophenyl)ethyl]-3-(phenylsulfonyl)benzenesulfonamide;
N-[2-(4-chlorophenyl)ethyl]-3-(phenylsulfonyl)benzenesulfonamide;
N-[2-(4-methoxyphenyl)ethyl]-3-(phenylsulfonyl)benzenesulfonamide;
N-[2-(4-methylphenyl)ethyl]-3-(phenylsulfonyl)benzenesulfonamide;
N-{2-[4-(aminosulfonyl)phenyl]ethyl}-3-(phenylsulfonyl)benzenesulfonamide;
N-{2-[3,4-bis(benzyloxy)phenyl]ethyl}-3-(phenylsulfonyl)benzenesulfonamide;
N-[2-(4-nitrophenyl)ethyl]-3-(phenylsulfonyl)benzenesulfonamide;
N-[2-(1,3-benzodioxol-5-yl)ethyl]-3-(phenylsulfonyl)benzenesulfonamide;
3-(phenylsulfonyl)-N-{2-[3-(trifluoromethyl)phenyl]ethyl}benzenesulfonamide;
N-[2-(3-chlorophenyl)ethyl]-3-(phenylsulfonyl)benzenesulfonamide;
N-[2-(2,4-dichlorophenyl)ethyl]-3-(phenylsulfonyl)benzenesulfonamide;
N-[2-(2,6-dichlorophenyl)ethyl]-3-(phenylsulfonyl)benzenesulfonamide;
N-[2-(2,5-dimethoxyphenyl)ethyl]-3-(phenylsulfonyl)benzenesulfonamide;
N-[2-(3,4-dichlorophenyl)ethyl]-3-(phenylsulfonyl)benzenesulfonamide;
N-[2-(3,5-dimethoxyphenyl)ethyl]-3-(phenylsulfonyl)benzenesulfonamide;
N-[2-(4-ethoxyphenyl)ethyl]-3-(phenylsulfonyl)benzenesulfonamide;
N-[2-(3-fluorophenyl)ethyl]-3-(phenylsulfonyl)benzenesulfonamide;
N-[2-(2-fluorophenyl)ethyl]-3-(phenylsulfonyl)benzenesulfonamide;
N-[2-(3-ethoxy-4-methoxyphenyl)ethyl]-3-(phenylsulfonyl)benzenesulfonamide;
N-[2-(4-ethoxy-3-methoxyphenyl)ethyl]-3-(phenylsulfonyl)benzenesulfonamide;
N-(4-methoxybenzyl)-3-(phenylsulfonyl)benzenesulfonamide;
N-[2-(2-bromo-4,5-dimethoxyphenyl)ethyl]-3-(phenylsulfonyl)benzenesulfonamide;
N-[2-(5-bromo-2-methoxyphenyl)ethyl]-3-(phenylsulfonyl)benzenesulfonamide;
N³-{[3-(phenylsulfonyl)phenyl]sulfonyl}-beta-alaninamide;
methyl N-{3-(phenylsulfonyl)phenyl]sulfonyl}-beta-alaninate;
N-(2-cyanoethyl)-3-(phenylsulfonyl)benzenesulfonamide;
3-(phenylsulfonyl)-N-(2-pyridin-2-ylethyl)benzenesulfonamide;
N-(3-morpholin-4-ylpropyl)-3-(phenylsulfonyl)benzenesulfonamide;
N-[2-(1-methylpyrrolidin-2-yl)ethyl]-3-(phenylsulfonyl)benzenesulfonamide;
3-(phenylsulfonyl)-N-(2-pyridin-4-ylethyl)benzenesulfonamide;
3-(phenylsulfonyl)-N-(2-piperidin-1-ylethyl)benzenesulfonamide;
N-[2-(1H-imidazol-4-yl)ethyl]-3-(phenylsulfonyl)benzenesulfonamide;
N-[2-({[3-(phenylsulfonyl)phenyl]sulfonyl}amino)ethyl]acetamide;
N-(2-morpholin-4-ylethyl)-3-(phenylsulfonyl)benzenesulfonamide;
N-[3-(2-oxopyrrolidin-1-yl)propyl]-3-(phenylsulfonyl)benzenesulfonamide;
N-[2-(diethylamino)ethyl]-3-(phenylsulfonyl)benzenesulfonamide;
N-[2-(dimethylamino)ethyl]-3-(phenylsulfonyl)benzenesulfonamide;
N-(3-methoxypropyl)-3-(phenylsulfonyl)benzenesulfonamide;
N-[3-(dimethylamino)propyl]-3-(phenylsulfonyl)benzenesulfonamide;
N-(2-methoxyethyl)-3-(phenylsulfonyl)benzenesulfonamide;
N-[3-(diethylamino)propyl]-3-(phenylsulfonyl)benzenesulfonamide;
N-[3-(1H-imidazol-1-yl)propyl]-3-(phenylsulfonyl)benzenesulfonamide;
3-(phenylsulfonyl)-N-(3-pyrrolidin-1-ylpropyl)benzenesulfonamide;
N-[3-(4-methylpiperazin-1-yl)propyl]-3-(phenylsulfonyl)benzenesulfonamide;
N-{3-(phenylsulfonyl)phenyl]sulfonyl}-beta-alanine;
N-2,3-dihydro-1H-inden-2-yl-3-(phenylsulfonyl)benzenesulfonamide;
N-[(1S,2R)-2-phenylcyclopropyl]-3-(phenylsulfonyl)benzenesulfonamide;
N-[(2R)-2-phenylpropyl]-3-(phenylsulfonyl)benzenesulfonamide;
N-[(2S)-2-phenylpropyl]-3-(phenylsulfonyl)benzenesulfonamide;
5-[(4-fluorophenyl)sulfonyl]-2-methyl-N-(2-phenylethyl)benzenesulfonamide;
N-[2-(2-chlorophenyl)ethyl]-5-[(4-fluorophenyl)sulfonyl]-2-methylbenzenesulfonamide;
5-[(4-fluorophenyl)sulfonyl]-N-[2-(2-methoxyphenyl)ethyl]-2-methylbenzenesulfonamide;
5-[(4-fluorophenyl)sulfonyl]-N-[2-(3-methoxyphenyl)ethyl]-2-methylbenzenesulfonamide;
N-[2-(3,4-dimethoxyphenyl)ethyl]-5-[(4-fluorophenyl)sulfonyl]-2-methylbenzenesulfonamide;
N-[2-(4-bromophenyl)ethyl]-5-[(4-fluorophenyl)sulfonyl]-2-methylbenzenesulfonamide;
N-[2-(4-fluorophenyl)ethyl]-5-[(4-fluorophenyl)sulfonyl]-2-methylbenzenesulfonamide;
N-[2-(4-chlorophenyl)ethyl]-5-[(4-fluorophenyl)sulfonyl]-2-methylbenzenesulfonamide;
5-[(4-fluorophenyl)sulfonyl]-N-[2-(4-methoxyphenyl)ethyl]-2-methylbenzenesulfonamide;
5-[(4-fluorophenyl)sulfonyl]-2-methyl-N-[2-(4-methylphenyl)ethyl]benzenesulfonamide;
N-{2-[4-(aminosulfonyl)phenyl]ethyl}-5-[(4-fluorophenyl)sulfonyl]-2-methylbenzenesulfonamide;
N-{2-[3,4-bis(benzyloxy)phenyl]ethyl}-5-[(4-fluorophenyl)sulfonyl]-2-methylbenzenesulfonamide;
5-[(4-fluorophenyl)sulfonyl]-2-methyl-N-[2-(4-nitrophenyl)ethyl]benzenesulfonamide;
N-[2-(1,3-benzodioxol-5-yl)ethyl]-5-[(4-fluorophenyl)sulfonyl]-2-methylbenzenesulfonamide;
5-[(4-fluorophenyl)sulfonyl]-2-methyl-N-{2-[3-(trifluoromethyl)phenyl]ethyl}benzenesulfonamide;
N-[2-(3-chlorophenyl)ethyl]-5-[(4-fluorophenyl)sulfonyl]-2-methylbenzenesulfonamide;
N-[2-(2,4-dichlorophenyl)ethyl]-5-[(4-fluorophenyl)sulfonyl]-2-methylbenzenesulfonamide;
N-[2-(2,6-dichlorophenyl)ethyl]-5-[(4-fluorophenyl)sulfonyl]-2-methylbenzenesulfonamide;
N-[2-(2,5-dimethoxyphenyl)ethyl]-5-[(4-fluorophenyl)sulfonyl]-2-methylbenzenesulfonamide;
N-[2-(3,4-dichlorophenyl)ethyl]-5-[(4-fluorophenyl)sulfonyl]-2-methylbenzenesulfonamide;
N-[2-(3,5-dimethoxyphenyl)ethyl]-5-[(4-fluorophenyl)sulfonyl]-2-methylbenzenesulfonamide;
N-[2-(4-ethoxyphenyl)ethyl]-5-[(4-fluorophenyl)sulfonyl]-2-methylbenzenesulfonamide;
N-[2-(3-fluorophenyl)ethyl]-5-[(4-fluorophenyl)sulfonyl]-2-methylbenzenesulfonamide;
N-[2-(2-fluorophenyl)ethyl]-5-[(4-fluorophenyl)sulfonyl]-2-methylbenzenesulfonamide;
N-[2-(3-ethoxy-4-methoxyphenyl)ethyl]-5-[(4-fluorophenyl)sulfonyl]-2-methylbenzenesulfonamide;
N-[2-(4-ethoxy-3-methoxyphenyl)ethyl]-5-[(4-fluorophenyl)sulfonyl]-2-methylbenzenesulfonamide;
5-[(4-fluorophenyl)sulfonyl]-N-(4-methoxybenzyl)-2-methylbenzenesulfonamide;
N-[2-(2-bromo-4,5-dimethoxyphenyl)ethyl]-5-[(4-fluorophenyl)sulfonyl]-2-methylbenzenesulfonamide;
N-[2-(5-bromo-2-methoxyphenyl)ethyl]-5-[(4-fluorophenyl)sulfonyl]-2-methylbenzenesulfonamide;
2-Methyl-N-(2-phenylethyl)-5-(phenylsulfonyl) benzene sulfonamide;
N-[2-(2-chlorophenyl)ethyl]-2-methyl-5-(phenylsulfonyl)benzenesulfonamide;
N-[2-(2-methoxyphenyl)ethyl]-2-methyl-5-(phenylsulfonyl)benzenesulfonamide;
N-[2-(3-methoxyphenyl)ethyl]-2-methyl-5-(phenylsulfonyl)benzenesulfonamide;
N-[2-(3,4-dimethoxyphenyl)ethyl]-2-methyl-5-(phenylsulfonyl)benzenesulfonamide;
N-[2-(4-bromophenyl)ethyl]-2-methyl-5-(phenylsulfonyl)benzenesulfonamide;
N-[2-(4-fluorophenyl)ethyl]-2-methyl-5-(phenylsulfonyl)benzenesulfonamide;
N-[2-(4-chlorophenyl)ethyl]-2-methyl-5-(phenylsulfonyl)benzenesulfonamide;
N-[2-(4-methoxyphenyl)ethyl]-2-methyl-5-(phenylsulfonyl)benzenesulfonamide;
2-methyl-N-[2-(4-methylphenyl)ethyl]-5-(phenylsulfonyl)benzenesulfonamide;
N-{2-[4-(aminosulfonyl)phenyl]ethyl}-2-methyl-5-(phenylsulfonyl)benzenesulfonamide;
N-{2-[3,4-bis(benzyloxy)phenyl]ethyl}-2-methyl-5-(phenylsulfonyl)benzenesulfonamide;
2-methyl-N-[2-(4-nitrophenyl)ethyl]-5-(phenylsulfonyl)benzenesulfonamide;
N-[2-(1,3-benzodioxol-5-yl)ethyl]-2-methyl-5-(phenylsulfonyl)benzenesulfonamide;
2-methyl-5-(phenylsulfonyl)-N-{2-[3-(trifluoromethyl)phenyl]ethyl}benzenesulfonamide;
N-[2-(3-chlorophenyl)ethyl]-2-methyl-5-(phenylsulfonyl)benzenesulfonamide;
N-[2-(2,4-dichlorophenyl)ethyl]-2-methyl-5-(phenylsulfonyl)benzenesulfonamide;
N-[2-(2,6-dichlorophenyl)ethyl]-2-methyl-5-(phenylsulfonyl)benzenesulfonamide;
N-[2-(2,5-dimethoxyphenyl)ethyl]-2-methyl-5-(phenylsulfonyl)benzenesulfonamide;
N-[2-(3,4-dichlorophenyl)ethyl]-2-methyl-5-(phenylsulfonyl)benzenesulfonamide;
N-[2-(3,5-dimethoxyphenyl)ethyl]-2-methyl-5-(phenylsulfonyl)benzenesulfonamide;
N-[2-(4-ethoxyphenyl)ethyl]-2-methyl-5-(phenylsulfonyl)benzenesulfonamide;
N-[2-(3-fluorophenyl)ethyl]-2-methyl-5-(phenylsulfonyl)benzenesulfonamide;
N-[2-(2-fluorophenyl)ethyl]-2-methyl-5-(phenylsulfonyl)benzenesulfonamide;
N-[2-(3-ethoxy-4-methoxyphenyl)ethyl]-2-methyl-5-(phenylsulfonyl)benzenesulfonamide;
N-[2-(4-ethoxy-3-methoxyphenyl)ethyl]-2-methyl-5-(phenylsulfonyl)benzenesulfonamide;
N-(4-methoxybenzyl)-2-methyl-5-(phenylsulfonyl)benzenesulfonamide;
N-[2-(2-bromo-4,5-dimethoxyphenyl)ethyl]-2-methyl-5-(phenylsulfonyl)benzenesulfonamide;
N-[2-(5-bromo-2-methoxyphenyl)ethyl]-2-methyl-5-(phenylsulfonyl)benzenesulfonamide;
5-[(4-chlorophenyl)sulfonyl]-2-methyl-N-(2-phenylethyl)benzenesulfonamide;
N-[2-(2-chlorophenyl)ethyl]-5-[(4-chlorophenyl)sulfonyl]-2-methylbenzenesulfonamide;
5-[(4-chlorophenyl)sulfonyl]-N-[2-(2-methoxyphenyl)ethyl]-2-methylbenzenesulfonamide;
5-[(4-chlorophenyl)sulfonyl]-N-[2-(3-methoxyphenyl)ethyl]-2-methylbenzenesulfonamide;
5-[(4-chlorophenyl)sulfonyl]-N-[2-(3,4-dimethoxyphenyl)ethyl]-2-methylbenzenesulfonamide;
N-[2-(4-bromophenyl)ethyl]-5-[(4-chlorophenyl)sulfonyl]-2-methylbenzenesulfonamide;
5-[(4-chlorophenyl)sulfonyl]-N-[2-(4-fluorophenyl)ethyl]-2-methylbenzenesulfonamide;
N-[2-(4-chlorophenyl)ethyl]-5-[(4-chlorophenyl)sulfonyl]-2-methylbenzenesulfonamide;
5-[(4-chlorophenyl)sulfonyl]-N-[2-(4-methoxyphenyl)ethyl]-2-methylbenzenesulfonamide;
5-[(4-chlorophenyl)sulfonyl]-2-methyl-N-[2-(4-methylphenyl)ethyl]benzenesulfonamide;
N-{2-[4-(aminosulfonyl)phenyl]ethyl}-5-[(4-chlorophenyl)sulfonyl]-2-methylbenzenesulfonamide;
5-[(4-chlorophenyl)sulfonyl]-2-methyl-N-[2-(4-nitrophenyl)ethyl]benzenesulfonamide;
N-(2-(1,3-benzodioxol-5-yl)ethyl]-5-[(4-chlorophenyl)sulfonyl]-2-methylbenzenesulfonamide;
5-[(4-chlorophenyl)sulfonyl]-2-methyl-N-{2-[3-(trifluoromethyl)phenyl]ethyl benzenesulfonamide;
N-[2-(3-chlorophenyl)ethyl]-5-[(4-chlorophenyl)sulfonyl]-2-methylbenzenesulfonamide;
5-[(4-chlorophenyl)sulfonyl]-N-[2-(2,4-dichlorophenyl)ethyl]-2-methylbenzenesulfonamide;
5-[(4-chlorophenyl)sulfonyl]-N-[2-(2,6-dichlorophenyl)ethyl]-2-methylbenzenesulfonamide;
5-[(4-chlorophenyl)sulfonyl]-N-[2-(2,5-dimethoxyphenyl)ethyl]-2-methylbenzenesulfonamide;
5-[(4-chlorophenyl)sulfonyl]-N-[2-(3,4-dichlorophenyl)ethyl]-2-methylbenzenesulfonamide;
5-[(4-chlorophenyl)sulfonyl]-N-[2-(3,5-dimethoxyphenyl)ethyl]-2-methylbenzenesulfonamide;
5-[(4-chlorophenyl)sulfonyl]-N-[2-(4-ethoxyphenyl)ethyl]-2-methylbenzenesulfonamide;
5-[(4-chlorophenyl)sulfonyl]-N-[2-(3-fluorophenyl)ethyl]-2-methylbenzenesulfonamide;
5-[(4-chlorophenyl)sulfonyl]-N-[2-(2-fluorophenyl)ethyl]-2-methylbenzenesulfonamide;
5-[(4-chlorophenyl)sulfonyl]-N-[2-(3-ethoxy-4-methoxyphenyl)ethyl]-2-methylbenzenesulfonamide;
5-[(4-chlorophenyl)sulfonyl]-N-[2-(4-ethoxy-3-methoxyphenyl)ethyl]-2-methylbenzenesulfonamide;
5-[(4-chlorophenyl)sulfonyl]-N-(4-methoxybenzyl)-2-methylbenzenesulfonamide;
N-[2-(2-bromo-4,5-dimethoxyphenyl)ethyl]-5-[(4-chlorophenyl)sulfonyl]-2-methylbenzenesulfonamide;
N-[2-(5-bromo-2-methoxyphenyl)ethyl]-5-[(4-chlorophenyl)sulfonyl]-2-methylbenzenesulfonamide;
2-methyl-5-[(4-methylphenyl)sulfonyl]-N-(2-phenylethyl)benzenesulfonamide;
N-[2-(2-chlorophenyl)ethyl]-2-methyl-5-[(4-methylphenyl)sulfonyl]benzenesulfonamide;
N-[2-(2-methoxyphenyl)ethyl]-2-methyl-5-[(4-methylphenyl)sulfonyl]benzenesulfonamide;
N-[2-(3-methoxyphenyl)ethyl]-2-methyl-5-[(4-methylphenyl)sulfonyl]benzenesulfonamide;
N-[2-(3,4-dimethoxyphenyl)ethyl]-2-methyl-5-[(4-methylphenyl)sulfonyl]benzenesulfonamide;
N-[2-(4-bromophenyl)ethyl]-2-methyl-5-[(4-methylphenyl)sulfonyl]benzenesulfonamide;
N-[2-(4-fluorophenyl)ethyl]-2-methyl-5-[(4-methylphenyl)sulfonyl]benzenesulfonamide;
N-[2-(4-chlorophenyl)ethyl]-2-methyl-5-[(4-methylphenyl)sulfonyl]benzenesulfonamide;
N-[2-(4-methoxyphenyl)ethyl]-2-methyl-5-[(4-methylphenyl)sulfonyl]benzenesulfonamide;
2-methyl-N-[2-(4-methylphenyl)ethyl]-5-[(4-methylphenyl)sulfonyl]benzenesulfonamide;
N-{2-[4-(aminosulfonyl)phenyl]ethyl}-2-methyl-5-[(4-methylphenyl)sulfonyl]benzenesulfonamide;
2-methyl-5-[(4-methylphenyl)sulfonyl]-N-[2-(4-nitrophenyl)ethyl]benzenesulfonamide;
N-[2-(1,3-benzodioxol-5-yl)ethyl]-2-methyl-5-[(4-methylphenyl)sulfonyl]benzenesulfonamide;
2-methyl-5-[(4-methylphenyl)sulfonyl]-N-{2-[3-(trifluoromethyl)phenyl]ethyl}benzenesulfonamide;
N-[2-(3-chlorophenyl)ethyl]-2-methyl-5-[(4-methylphenyl)sulfonyl]benzenesulfonamide;
N-[2-(2,4-dichlorophenyl)ethyl]-2-methyl-5-[(4-methylphenyl)sulfonyl]benzenesulfonamide;
N-[2-(2,6-dichlorophenyl)ethyl]-2methyl-5-[(4-methylphenyl)sulfonyl]benzenesulfonamide;
N-[2-(2,5-dimethoxyphenyl)ethyl]-2-methyl-5-[(4-methylphenyl)sulfonyl]benzenesulfonamide;
N-[2-(3,4-dichlorophenyl)ethyl]-2-methyl-5-[(4-methylphenyl)sulfonyl]benzenesulfonamide;
N-[2-(3,5-dimethoxyphenyl)ethyl]-2-methyl-5-[(4-methylphenyl)sulfonyl]benzenesulfonamide;
N-[2-(4-ethoxyphenyl)ethyl]-2-methyl-5-[(4-methylphenyl)sulfonyl]benzenesulfonamide;
N-[2-(3-fluorophenyl)ethyl]-2-methyl-5-[(4-methylphenyl)sulfonyl]benzenesulfonamide;
N-[2-(2-fluorophenyl)ethyl]-2-methyl-5-[(4-methylphenyl)sulfonyl]benzenesulfonamide;
N-[2-(2-bromo-4,5-dimethoxyphenyl)ethyl]-2-methyl-5-[(4-methylphenyl)sulfonyl]benzenesulfonamide;
N-[2-(5-bromo-2-methoxyphenyl)ethyl]-2-methyl-5-[(4-methylphenyl)sulfonyl]benzenesulfonamide;
2-ethyl-N-(2-phenylethyl)-5-(phenylsulfonyl)benzenesulfonamide;
2-ethyl-N-[2-(2-methoxyphenyl)ethyl]-5-(phenylsulfonyl)benzenesulfonamide;
2-ethyl-N-[2-(3-methoxyphenyl)ethyl]-5-(phenylsulfonyl)benzenesulfonamide;
N-[2-(3,4-dimethoxyphenyl)ethyl]-2-ethyl-5-(phenylsulfonyl)benzenesulfonamide;
N-{2-[4-(aminosulfonyl)phenyl]ethyl}-2-ethyl-5-(phenylsulfonyl)benzenesulfonamide;
2-ethyl-N-[2-(3-fluorophenyl)ethyl]-5-(phenylsulfonyl)benzenesulfonamide;
2-ethyl-N-[2-(2-fluorophenyl)ethyl]-5-(phenylsulfonyl)benzenesulfonamide;
N-[2-(3-ethoxy-4-methoxyphenyl)ethyl]-2-ethyl-5-(phenylsulfonyl)benzenesulfonamide;
N-[2-(4-ethoxy-3-methoxyphenyl)ethyl]-2-ethyl-5-(phenylsulfonyl)benzenesulfonamide;
2-methoxy-N-(2-phenylethyl)-5-(phenylsulfonyl)benzenesulfonamide;
2-methoxy-N-[2-(2-methoxyphenyl)ethyl]-5-(phenylsulfonyl)benzenesulfonamide;
2-methoxy-N-[2-(3-methoxyphenyl)ethyl]-5-(phenylsulfonyl)benzenesulfonamide;
N-[2-(3,4-dimethoxyphenyl)ethyl]-2-methoxy-5-(phenylsulfonyl)benzenesulfonamide;
N-{2-[4-(aminosulfonyl)phenyl]ethyl}-2-methoxy-5-(phenylsulfonyl)benzenesulfonamide;
N-[2-(3-fluorophenyl)ethyl]-2-methoxy-5-(phenylsulfonyl)benzenesulfonamide;
N-[2-(2-fluorophenyl)ethyl]-2-methoxy-5-(phenylsulfonyl)benzenesulfonamide;
N-[2-(3-ethoxy-4-methoxyphenyl)ethyl]-2-methoxy-5-(phenylsulfonyl)benzenesulfonamide;
N-[2-(4-ethoxy-3-methoxyphenyl)ethyl]-2-methoxy-5-(phenylsulfonyl)benzenesulfonamide;
2-methyl-N-(3-phenylpropyl)-5-(phenylsulfonyl)benzenesulfonamide;
2-ethyl-N-(3-phenylpropyl)-5-(phenylsulfonyl)benzenesulfonamide;
N-2,3-dihydro-1H-inden-2-yl-2-methyl-5-(phenylsulfonyl)benzenesulfonamide;
2-methyl-N-[(1S,2R)-2-phenylcyclopropyl]-5-(phenylsulfonyl)benzenesulfonamide;
2-methyl-N-[(2R)-2-phenylpropyl]-5-(phenylsulfonyl)benzenesulfonamide;
N-2,3-dihydro-1H-inden-2-yl-5-[(4-fluorophenyl)sulfonyl]-2-methylbenzenesulfonamide;
5-[(4-fluorophenyl)sulfonyl]-2-methyl-N-[(1S,2R)-2-phenylcyclopropyl]benzenesulfonamide;
5-[(4-fluorophenyl)sulfonyl]-2-methyl-N-[(2R)-2-phenylpropyl]benzenesulfonamide;
N-2,3-dihydro-1H-inden-2-yl-2-methyl-5-[(4-methylphenyl)sulfonyl]benzenesulfonamide;
2-methyl-5-[(4-methylphenyl)sulfonyl]-N-[(1S,2R)-2-phenylcyclopropyl]benzenesulfonamide;
2-methyl-5-[(4-methylphenyl)sulfonyl]-N-[(2R)-2-phenylpropyl]benzenesulfonamide;
5-[(4-chlorophenyl)sulfonyl]-N-2,3-dihydro-1H-inden-2-yl-2-methylbenzenesulfonamide;
5-[(4-chlorophenyl)sulfonyl]-2-methyl-N-[(1S,2R)-2-phenylcyclopropyl]benzenesulfonamide;
5-[(4-chlorophenyl)sulfonyl]-2-methyl-N-[(2R)-2-phenylpropyl]benzenesulfonamide;
5-[(4-fluorophenyl)sulfonyl]-2-methyl-N-[(2S)-2-phenylpropyl]benzenesulfonamide;
2-methyl-N-[(2S)-2-phenylpropyl]-5-(phenylsulfonyl)benzenesulfonamide;
2-methyl-5-[(4-methylphenyl)sulfonyl]-N-[(2S)-2-phenylpropyl]benzenesulfonamide;
5-[(4-chlorophenyl)sulfonyl]-2-methyl-N-[(2S)-2-phenylpropyl]benzenesulfonamide;
N-2,3-dihydro-1H-inden-2-yl-2-ethyl-5-(phenylsulfonyl)benzenesulfonamide;
2-ethyl-N-[(1S,2R)-2-phenylcyclopropyl]-5-(phenylsulfonyl)benzenesulfonamide;
2-ethyl-N-[(2R)-2-phenylpropyl]-5-(phenylsulfonyl)benzenesulfonamide;
N-2,3-dihydro-1H-inden-2-yl-2-methoxy-5-(phenylsulfonyl)benzenesulfonamide;
2-methoxy-N-[(1S,2R)-2-phenylcyclopropyl]-5-(phenylsulfonyl)benzenesulfonamide;
2-methoxy-N-[(2R)-2-phenylpropyl]-5-(phenylsulfonyl)benzenesulfonamide;
2-isopropyl-N-(2-phenylethyl)-5-(phenylsulfonyl)benzenesulfonamide;
2-methyl-N-(2-phenylethyl)-3-(phenylsulfonyl)benzenesulfonamide;
2-chloro-N-(2-phenylethyl)-5-(phenylsulfonyl)benzenesulfonamide;
2-(2-hydroxy-2-methylpropyl)-N-(2-phenylethyl)-5-(phenylsulfonyl)benzenesulfonamide;
N-{[2-methyl-5-(phenylsulfonyl)phenyl]sulfonyl}-L-phenylalaninamide;
methyl N-{2-methyl-5-(phenylsulfonyl)phenyl]sulfonyl}-D-phenylalaninate;
N-(2,3-dihydro-1H-inden-1-yl)-2-methyl-5-(phenylsulfonyl)benzenesulfonamide;
N-[2-(2-fluorophenyl)ethyl]-3-[(4-methylphenyl)sulfonyl]-5,6,7,8-tetrahydronaphthalene-1-sulfonamide;
5-[(4-fluorophenyl)sulfonyl]-2-isopropyl-N-[(2R)-2-phenylpropyl]benzenesulfonamide;
5-[(4-fluorophenyl)sulfonyl]-2-isopropyl-N-[(2S)-2-phenylpropyl]benzenesulfonamide;
N-(2-phenylethyl)-5-(phenylsulfonyl)-2-propylbenzenesulfonamide;
5-(phenylsulfonyl)-2-propylbenzenesulfonamide;
5-[(4-fluorophenyl)sulfonyl]-N-(2-phenylethyl)-2-propylbenzenesulfonamide;
5-[(4-fluorophenyl)sulfonyl]-2-propylbenzenesulfonamide;
N-(2-phenylethyl)-5-(phenylsulfonyl)-2-(trifluoromethyl)-benzenesulfonamide;
2,4-diisopropyl-N-(2-phenylethyl)-5-(phenylsulfonyl)benzenesulfonamide;
N-(2,3-dihydro-1H-inden-2-yl)-2,4-diisopropyl-5-(phenylsulfonyl)benzenesulfonamide;
5-[(4-fluorophenyl)sulfonyl]-2,4-diisopropyl-N-(2-phenylethyl)benzenesulfonamide;
N-(2,3-dihydro-1H-inden-2-yl)-5-[(4-fluorophenyl)sulfonyl]-2,4-diisopropylbenzenesulfonamide;
N-(2-hydroxy-2-phenylethyl)-2-methyl-5-(phenylsulfonyl)benzenesulfonamide;
5-[(4-fluorophenyl)sulfonyl]-N-(2-hydroxy-2-phenylethyl)-2-methylbenzenesulfonamide;
2-ethyl-5-[(4-fluorophenyl)sulfonyl]-N-(2-hydroxy-2-phenylethyl)benzenesulfonamide;
N-(2-hydroxy-2-phenylethyl)-2,4-dimethyl-5-(phenylsulfonyl)benzenesulfonamide;
trans-N-(2-hydroxy-1-methyl-2-phenylethyl)-2-methyl-5-(phenylsulfonyl)benzenesulfonamide;
5-[(4-fluorophenyl)sulfonyl]-N-[trans-2-hydroxy-1-methyl-2-phenylethyl]-2-methylbenzenesulfonamide;
2-ethyl-5-[(4-fluorophenyl)sulfonyl]-N-[(trans)-2-hydroxy-1-methyl-2-phenylethyl]benzenesulfonamide;
N-[(trans)-2-hydroxy-1-methyl-2-phenylethyl]-2,4-dimethyl-5-(phenylsulfonyl)benzenesulfonamide;
N-[(1S*,2S*)-2-hydroxycyclohexyl]-2-methyl-5-(phenylsulfonyl)benzenesulfonamide;
5-[(4-fluorophenyl)sulfonyl]-N-(2-hydroxyethyl)-2-isopropylbenzenesulfonamide;
5-[(4-fluorophenyl)sulfonyl]-2-methyl-N-(2-pyridin-2-ylethyl)benzenesulfonamide;
5-[(4-fluorophenyl)sulfonyl]-2-methyl-N-(3-morpholin-4-ylpropyl)benzenesulfonamide;
5-[(4-fluorophenyl)sulfonyl]-2-methyl-N-(2-pyridin-4-ylethyl)benzenesulfonamide;
2-methyl-5-(phenylsulfonyl)-N-(2-pyridin-2-ylethyl)benzenesulfonamide;
2-methyl-5-(phenylsulfonyl)-N-(2-pyridin-4-ylethyl)benzenesulfonamide;
2-ethyl-5-(phenylsulfonyl)-N-(2-pyridin-2-ylethyl)benzenesulfonamide;
2-methoxy-5-(phenylsulfonyl)-N-(2-pyridin-2-ylethyl)benzenesulfonamide;
2-methoxy-5-(phenylsulfonyl)-N-(2-pyridin-4-ylethyl)benzenesulfonamide;
5-[(4-chlorophenyl)sulfonyl]-2-methyl-N-(2-pyridin-2-ylethyl)benzenesulfonamide;
5-[(4-fluorophenyl)sulfonyl]-2-isopropyl-N-(2-pyridin-2-ylethyl)benzenesulfonamide;
2,4-dimethyl-5-(phenylsulfonyl)-N-(2-pyridin-2-ylethyl)benzenesulfonamide;
5-[(4-fluorophenyl)sulfonyl]-2,4-dimethyl-N-(2-pyridin-2-ylethyl)benzenesulfonamide;
2-chloro-5-(phenylsulfonyl)-N-(2-pyridin-2-ylethyl)benzenesulfonamide;
2,3-dimethyl-5-(phenylsulfonyl)-N-(2-pyridin-2-ylethyl)benzenesulfonamide;
2-methyl-5-{4-(methylamino)phenyl]sulfonyl}-N-(2-pyridin-2-ylethyl)benzenesulfonamide;
2-isopropyl-5-{4-(methylamino)phenyl]sulfonyl}-N-(2-pyridin-2-ylethyl)benzenesulfonamide;
5-{[4-(1-cyclohexyl-1-hydroxyethyl)phenyl]sulfonyl}-2-methyl-N-(2-pyridin-2-ylethyl)benzenesulfonamide;
5-{4-(1-hydroxy-1-phenylethyl)phenyl]sulfonyl}2-methyl-N-(2-pyridin-2-ylethyl)benzenesulfonamide;
5-{4-(1-hydroxy-1-methyl-2-phenylethyl)phenyl]sulfonyl}-2-methyl-N-(2-pyridin-2-ylethyl)benzenesulfonamide;
5-[(3-cyanophenyl)sulfonyl]-2-methyl-N-(2-pyridin-2-ylethyl)benzenesulfonamide;
2-methyl-5-(1-naphthylsulfonyl)-N-(2-pyridin-2-ylethyl)benzenesulfonamide;
5-[(3-hydroxyphenyl)sulfonyl]-2-methyl-N-(2-pyridin-2-ylethyl)benzenesulfonamide;
5-[(3,5-difluorophenyl)sulfonyl]-2-methyl-N-(2-pyridin-2-ylethyl)benzenesulfonamide;
5-[(4-ethylphenyl)sulfonyl]-2-methyl-N-(2-pyridin-2-ylethyl)benzenesulfonamide;
5-[(3-acetylphenyl)sulfonyl]-2-methyl-N-(2-pyridin-2-ylethyl)benzenesulfonamide;
5-[(2-ethoxyphenyl)sulfonyl]-2-methyl-N-(2-pyridin-2-ylethyl)benzenesulfonamide;
5-[(2,5-dimethoxyphenyl)sulfonyl]-2-methyl-N-(2-pyridin-2-ylethyl)benzenesulfonamide;
5-[(2,3-dimethoxyphenyl)sulfonyl]-2-methyl-N-(2-pyridin-2-ylethyl)benzenesulfonamide;
5-[(2,4-dimethoxyphenyl)sulfonyl]-2-methyl-N-(2-pyridin-2-ylethyl)benzenesulfonamide;
2-methyl-N-(2-pyridin-2-ylethyl)-5-(pyridin-3-ylsulfonyl)benzenesulfonamide;
5-(1H-indol-5-ylsulfonyl)-2-methyl-N-(2-pyridin-2-ylethyl)benzenesulfonamide;
5-[(4-cyanophenyl)sulfonyl]-2-methyl-N-(2-pyridin-2-ylethyl)benzenesulfonamide;
5-{3-(ethylsulfonyl)phenyl]sulfonyl}-2-methyl-N-(2-pyridin-2-ylethyl)benzenesulfonamide;
2-methyl-5-[(2-methylphenyl)sulfonyl]-N-(2-pyridin-2-ylethyl)benzenesulfonamide;
5-[(2-ethylphenyl)sulfonyl]-2-methyl-N-(2-pyridin-2-ylethyl)benzenesulfonamide;
5-(biphenyl-2-ylsulfonyl)-2-methyl-N-(2-pyridin-2-ylethyl)benzenesulfonamide;
5-(biphenyl-4-ylsulfonyl)-2-methyl-N-(2-pyridin-2-ylethyl)benzenesulfonamide;
5-(biphenyl-3-ylsulfonyl)-2-methyl-N-(2-pyridin-2-ylethyl)benzenesulfonamide;
5-[(4-tert-butylphenyl)sulfonyl]-2-isopropyl-N-(2-pyridin-2-ylethyl)benzenesulfonamide;
5-[(4-tert-butylphenyl)sulfonyl]-2-methyl-N-(2-pyridin-2-ylethyl)benzenesulfonamide;
5-[(3,5-dimethylphenyl)sulfonyl]-2-isopropyl-N-(2-pyridin-2-ylethyl)benzenesulfonamide;
5-[(3-chlorophenyl)sulfonyl]-2-isopropyl-N-(2-pyridin-2-ylethyl)benzenesulfonamide;
2-isopropyl-5-[(3-methoxyphenyl)sulfonyl]-N-(2-pyridin-2-ylethyl)benzenesulfonamide;
2-isopropyl-5-[(2-methoxyphenyl)sulfonyl]-N-(2-pyridin-2-ylethyl)benzenesulfonamide;
5-[(3,5-difluorophenyl)sulfonyl]-2-isopropyl-N-(2-pyridin-2-ylethyl)benzenesulfonamide;
2-cyclohexyl-5-(phenylsulfonyl)-N-(2-pyridin-2-ylethyl)benzenesulfonamide;
2-cydlohexyl-5-[(4-fluorophenyl)sulfonyl]-N-(2-pyridin-2-ylethyl)benzenesulfonamide;
2-tert-butyl-5-(phenylsulfonyl)-N-(2-pyridin-2-ylethyl)benzenesulfonamide;
2,6-dimethyl-3-(phenylsulfonyl)-N-(2-pyridin-2-ylethyl)benzenesulfonamide;
5-{5-(dimethylamino)-1-naphthyl]sulfonyl}-2-isopropyl-N-(2-pyridin-2-ylethyl)benzenesulfonamide;
5-[(3-chloro-5-cyanophenyl)sulfonyl]-2-methyl-N-(2-pyridin-2-ylethyl)benzenesulfonamide 2-methyl-3-(phenylsulfonyl)-N-(2-pyridin-2-ylethyl)benzenesulfonamide;
5-[(3,5-dichlorophenyl)sulfonyl]-2-isopropyl-N-(2-pyridin-2-ylethyl)benzenesulfonamide;
2-isopropyl-N-(2-pyridin-2-ylethyl)-5-{3-(trifluoromethoxy)phenyl]sulfonyl}benzenesulfonamide;
5-{[4-(dimethylamino)phenyl]sulfonyl}-2-isopropyl-N-(2-pyridin-2-ylethyl)benzenesulfonamide;
2-isopropyl-N-(2-pyridin-2-ylethyl)-5-{[3-(trifluoromethyl)phenyl]sulfonyl}benzenesulfonamide;
5-[(5-chloro-2-methoxyphenyl)sulfonyl]-2-isopropyl-N-(2-pyridin-2-ylethyl)benzenesulfonamide;
2-isopropyl-N-(2-pyridin-2-ylethyl)-5-(quinolin-8-ylsulfonyl)benzenesulfonamide;
5-[(2,5-dichloro-3-thienyl)sulfonyl]-2-isopropyl-N-(2-pyridin-2-ylethyl)benzenesulfonamide;
5-[(5-chloro-1,3-dimethyl-1H-pyrazol-4-yl)sulfonyl]-2-isopropyl-N-(2-pyridin-2-ylethyl)benzenesulfonamide;
2-isopropyl-5-[(1-methyl-1H-imidazol4-yl)sulfonyl]-N-(2-pyridin-2-ylethyl)benzenesulfonamide;
5-[(3-chloro-2-methylphenyl)sulfonyl]-2-isopropyl-N-(2-pyridin-2-ylethyl)benzenesulfonamide;
5-[(4,4-dimethyl-2-oxo-1,4-dihydro-2H-3,1-benzoxazin-6-yl)sulfonyl]-2-isopropyl-N-(2-pyridin-2-ylethyl)benzenesulfonamide;
5-[(4-fluorophenyl)sulfonyl]-2-isopropyl-N-(2-pyridin-4-ylethyl)benzenesulfonamide;
5-[(4-fluorophenyl)sulfonyl]-2-isopropyl-N-(2-pyridin-3-ylethyl)benzenesulfonamide;
5-[(3-cyanophenyl)sulfonyl]-2-isopropyl-N-(2-pyridin-2-ylethyl)benzenesulfonamide;
5-(1H-indol-5-ylsulfonyl)-2-isopropyl-N-(2-pyridin-2-ylethyl)benzenesulfonamide;
5-(phenylsulfonyl)-2-propyl-N-(2-pyridin-2-ylethyl)benzenesulfonamide;
5-[(4-fluorophenyl)sulfonyl]-2-propyl-N-(2-pyridin-2-ylethyl)benzenesulfonamide;
5-(phenylsulfonyl)-N-(2-pyridin-2-ylethyl)-2-(trifluoromethyl)benzenesulfonamide;
2-isopropyl-5-(phenylsulfinyl)-N-(2-pyridin-2-ylethyl)benzenesulfonamide;
5-[(3-bromo-2-methylphenyl)sulfonyl]-2-isopropyl-N-(2-pyridin-2-ylethyl)benzenesulfonamide;
5-[(2-chloro-6-methylphenyl)sulfonyl]-2-isopropyl-N-(2-pyridin-2-ylethyl)benzenesulfonamide;
2-isopropyl-5-[(3-methylphenyl)sulfonyl]-N-(2-pyridin-2-ylethyl)benzenesulfonamide;
2-isopropyl-5-[(R)-phenylsulfinyl]-N-(2-pyridin-2-ylethyl)benzenesulfonamide;
2-isopropyl-5-[(S)-phenylsulfinyl]-N-(2-pyridin-2-ylethyl)benzenesulfonamide;
2-bromo-5-(phenylsulfonyl)-N-(2-pyridin-2-ylethyl)benzenesulfonamide;
5-[(3-cyano-2-methylphenyl)sulfonyl]-2-isopropyl-N-(2-pyridin-2-ylethyl)benzenesulfonamide;
5-[(3-acetyl-2-methylphenyl)sulfonyl]-2-isopropyl-N-(2-pyridin-2-ylethyl)benzenesulfonamide;
5-[(3-chloro-4-fluorophenyl)sulfonyl]-2-isopropyl-N-(2-pyridin-2-ylethyl)benzenesulfonamide;
5-[(4-fluoro-2-methylphenyl)sulfonyl]-2-isopropyl-N-(2-pyridin-2-ylethyl)benzenesulfonamide;
2-isopropyl-5-[(1-methyl-1H-indol-5-yl)sulfonyl]-N-(2-pyridin-2-ylethyl)benzenesulfonamide;
2-isopropyl-5-{2-methyl-4-(methylamino)phenyl]sulfonyl}-N-(2-pyridin-2-ylethyl)benzenesulfonamide;
5-{3-chloro-4-(methylamino)phenyl]sulfonyl}-2-isopropyl-N-(2-pyridin-2-ylethyl)benzenesulfonamide;
2,4-diisopropyl-5-(phenylsulfonyl)-N-(2-pyridin-2-ylethyl)benzenesulfonamide;
2,4-diisopropyl-5-(phenylsulfonyl)-N-(2-pyridin-3-ylethyl)benzenesulfonamide;
2,4-diisopropyl-5-(phenylsulfonyl)-N-(2-pyridin-4-ylethyl)benzenesulfonamide;
5-[(4-fluorophenyl)sulfonyl]-2,4-diisopropyl-N-(2-pyridin-2-ylethyl)benzenesulfonamide;
5-[(4-fluorophenyl)sulfonyl]-2,4-diisopropyl-N-(2-pyridin-3-ylethyl)benzenesulfonamide;
5-[(4-fluorophenyl)sulfonyl]-2,4-diisopropyl-N-(2-pyridin-4-ylethyl)benzenesulfonamide;
2-chloro-5-(phenylsulfonyl)-N-(2-pyridin-3-ylethyl)benzenesulfonamide;
2-chloro-5-(phenylsulfonyl)-N-(2-pyridin-4-ylethyl)benzenesulfonamide;
5-[(4-fluorophenyl)sulfonyl]-2-isopropyl-N-[2-(l1-oxidopyridin-3-yl)ethyl]benzenesulfonamide;
5-[(4-fluorophenyl)sulfonyl]-N-[2-(1H-imidazol-4-yl)ethyl]-2-methylbenzenesulfonamide;
5-[(4-fluorophenyl)sulfonyl]-N-[3-(1H-imidazol-1-yl)propyl]-2-methylbenzenesulfonamide;
N-[2-(1H-imidazol-4-yl)ethyl]-2-methyl-5-(phenylsulfonyl)benzenesulfonamide;
N-[3-(1H-imidazol-1-yl)propyl]-2-methyl-5-(phenylsulfonyl)benzenesulfonamide;
2-ethyl-N-[2-(1H-imidazol-4-yl)ethyl]-5-(phenylsulfonyl)benzenesulfonamide;
2-ethyl-N-[3-(1H-imidazol-1-yl)propyl]-5-(phenylsulfonyl)benzenesulfonamide;
N-[2-(1H-imidazol-4-yl)ethyl]-2-methoxy-5-(phenylsulfonyl)benzenesulfonamide;
N-[3-(1H-imidazol-1-yl)propyl]-2-methoxy-5-(phenylsulfonyl)benzenesulfonamide;
5-[(4-chlorophenyl)sulfonyl]-N-[3-(1H-imidazol-1-yl)propyl]-2-methylbenzenesulfonamide;
5-[(4-azidophenyl)sulfonyl]-N-[3-(1H-imidazol-1-yl)propyl]-2-methylbenzenesulfonamide;
N-[2-(1H-indol-3-yl)ethyl]-2-methyl-5-(phenylsulfonyl)benzenesulfonamide;
N-[3-(1H-imidazol-1-yl)propyl]-2-isopropyl-5-(phenylsulfonyl)benzenesulfonamide;
2-ethyl-5-[(4-fluorophenyl)sulfonyl]-N-[2-(1H-imidazol-1-yl)ethyl]benzenesulfonamide;
2-ethyl-5-[(4-fluorophenyl)sulfonyl]-N-[3-(1H-imidazol-1-yl)propyl]benzenesulfonamide;
5-({4-[ethyl(methyl)amino]phenyl}sulfonyl)-N-[3-(1H-imidazol-1-yl)propyl]-2-methylbenzenesulfonamide;
N-[3-(1H-imidazol-1-yl)propyl]-2-methyl-5-[(4-pyrrolidin-1-ylphenyl)sulfonyl]benzenesulfonamide;
N-[3-(1H-imidazol-1-yl)propyl]-4-methyl-3-(phenylsulfinyl)benzenesulfonamide;
5-[(4-fluorophenyl)sulfonyl]-N-[2-(1H-imidazol-1-yl)ethyl]-2-methylbenzenesulfonamide;
N-[3-(1H-imidazol-1-yl)propyl]-2-methyl-5-{4-(methylamino)phenyl]sulfonyl}benzenesulfonamide;
5-{4-(ethylamino)phenyl]sulfonyl}-N-[3-(1H-imidazol-1-yl)propyl]-2-methylbenzenesulfonamide;
N-[3-(1H-imidazol-1-yl)propyl]-2-methyl-5-[(4-piperidin-1-ylphenyl)sulfonyl]benzenesulfonamide;
N-[3-(1H-imidazol-1-yl)propyl]-2-methyl-5-[(4-morpholin-4-ylphenyl)sulfonyl]benzenesulfonamide;
N-[2-(1H-imidazol-1-yl)ethyl]-2-methyl-5-(phenylsulfonyl)benzenesulfonamide;
N-[3-(1H-imidazol-1-yl)propyl]-5-[(4-methoxyphenyl)sulfonyl]-2-methylbenzenesulfonamide;
N-[3-(1H-imidazol-1-yl)propyl]-5-[(2-methoxyphenyl)sulfonyl]-2-methylbenzenesulfonamide;
N-[3-(1H-imidazol-1-yl)propyl]-4-methyl-3-(phenylsulfonyl)benzenesulfonamide;
5-[(4-fluorophenyl)sulfonyl]-N-[2-(1H-imidazol-1-yl)ethyl]-2-isopropylbenzenesulfonamide;
5-[(4-fluorophenyl)sulfonyl]-N-[3-(1H-imidazol-1-yl)propyl]-2-isopropylbenzenesulfonamide;
N-[3-(1H-imidazol-1-yl)propyl]-5-[(3-methoxyphenyl)sulfonyl]-2-methylbenzenesulfonamide;
N-[2-(1H-imidazol-1-yl)ethyl]-2,4-dimethyl-5-(phenylsulfonyl)benzenesulfonamide;
N-[3-(1H-imidazol-1-yl)propyl]-2,4-dimethyl-5-(phenylsulfonyl)benzenesulfonamide;
5-[(4-fluorophenyl)sulfonyl]-N-[2-(1H-imidazol-1-yl)ethyl]-2,4-dimethylbenzenesulfonamide;
5-[(4-fluorophenyl)sulfonyl]-N-[3-(1H-imidazol-1-yl)propyl]-2,4-dimethylbenzenesulfonamide;
2-chloro-N-[3-(1H-imidazol-1-yl)propyl]-5-(phenylsulfonyl)benzenesulfonamide;
N-[3-(1H-imidazol-1-yl)propyl]-5-[(4-isopropylphenyl)sulfonyl]-2-methylbenzenesulfonamide;
N-[3-(1H-imidazol-1-yl)propyl]-2-methyl-5-(2-naphthylsulfonyl)benzenesulfonamide;
5-[(3,4-dichlorophenyl)sulfonyl]-N-[3-(1H-imidazol-1-yl)propyl]-2-methylbenzenesulfonamide;
5-[(3-chlorophenyl)sulfonyl]-N-[3-(1H-imidazol-1-yl)propyl]-2-methylbenzenesulfonamide;
5-[(3,5-dimethylphenyl)sulfonyl]-N-[3-(1H-imidazol-1-yl)propyl]-2-methylbenzenesulfonamide;
5-[(3,5-dichlorophenyl)sulfonyl]-N-[3-(1H-imidazol-1-yl)propyl]-2-methylbenzenesulfonamide;
5-[(2,5-dichlorophenyl)sulfonyl]-N-[3-(1H-imidazol-1-yl)propyl]-2-methylbenzenesulfonamide;
N-[3-(1H-imidazol-1-yl)propyl]-2-methyl-5-(phenylsulfinyl)benzenesulfonamide;
N-[2-(1H-imidazol-1-yl)ethyl]-2,3-dimethyl-5-(phenylsulfonyl)benzenesulfonamide;
N-[3-(1H-imidazol-1-yl)propyl]-2,3-dimethyl-5-(phenylsulfonyl)benzenesulfonamide;
5-{4-(cyclohexylamino)phenyl]sulfonyl}-N-[3-(1H-imidazol-1-yl)propyl]-2-methylbenzenesulfonamide;
5-({4-[(2-cyanoethyl)amino]phenyl}sulfonyl)-N-[3-(1H-imidazol-1-yl)propyl]-2-methylbenzenesulfonamide;
5-[(4-{[(1S,2S)-1-(hydroxymethyl)-2-methylbutyl]amino}phenyl)sulfonyl]-N-[3-(1H-imidazol-1-yl)propyl]-2-methylbenzenesulfonamide;
N-[3-(1H-imidazol-1-yl)propyl]-2-methyl-5-({4-[(1-phenylethyl)amino]phenyl}sulfonyl)benzenesulfonamide;
5-[(2,3-dichlorophenyl)sulfonyl]-N-[3-(1H-imidazol-1-yl)propyl]-2-methylbenzenesulfonamide;
N-[3-(1H-imidazol-1-yl)propyl]-2-methyl-5-(2-thienylsulfonyl)benzenesulfonamide;
N-[3-(1H-imidazol-1-yl)propyl]-2-methyl-5-[(2-methyl-3-furyl)sulfonyl]benzenesulfonamide;
N-[3-(1H-imidazol-1-yl)propyl]-2-methyl-5-{[4-(tetrahydro-2H-pyran-4-ylamino)phenyl]sulfonyl}benzenesulfonamide;
N-[3-(1H-imidazol-1-yl)propyl]-5-({4-[(3-isopropoxypropyl)amino]phenyl}sulfonyl)-2-methylbenzenesulfonamide;
5-({4-[(cyclopropylmethyl)amino]phenyl}sulfonyl)-N-[3-(1H-imidazol-1-yl)propyl]-2-methylbenzenesulfonamide;
5-({4-[(1R,2R,4S)-bicyclo[2.2.1]hept-2-ylamino]phenyl}sulfonyl)-N-[3-(1H-imidazol-1-yl)propyl]-2-methylbenzenesulfonamide;
5-{4-(benzylamino)phenyl]sulfonyl}-N-[3-(1H-imidazol-1-yl)propyl]-2-methylbenzenesulfonamide;
5-[(4-{[(1 S)-1-cyclohexylethyl]amino }phenyl)sulfonyl]-N-[3-(1H-imidazol-1-yl)propyl]-2-methylbenzenesulfonamide;
5-[(4-{[(1R)-1-cyclohexylethyl]amino}phenyl)sulfonyl]-N-[3-(1H-imidazol-1-yl)propyl]-2-methylbenzenesulfonamide;
5-({4-[(2-hydroxybutyl)amino]phenyl}sulfonyl)-N-[3-(1H-imidazol-1-yl)propyl]-2-methylbenzenesulfonamide;
N-[3-(1H-imidazol-1-yl)propyl]-2-methyl-5-[(4-{4-(trifluoromethyl)benzyl]amino}phenyl)sulfonyl]benzenesulfonamide;
5-[(2-chlorophenyl)sulfonyl]-N-[3-(1H-imidazol-1-yl)propyl]-2-methylbenzenesulfonamide;
N-(tert-butyl)-2-methyl-5-(phenylsulfonyl)benzenesulfonamide;
5-[(4-tert-butylphenyl)sulfonyl]-N-[3-(1H-imidazol-1-yl)propyl]-2-isopropylbenzenesulfonamide;
5-[(4-tert-butylphenyl)sulfonyl]-N-[3-(1H-imidazol-1-yl)propyl]-2-methylbenzenesulfonamide;
5-[(4-fluorophenyl)sulfonyl]-N-[2-(1H-imidazol4-yl)ethyl]-2-isopropylbenzenesulfonamide;
5-({4-[(2-cyanoethyl)amino]phenyl}sulfonyl)-N-[2-(1H-imidazol-1-yl)ethyl]-2-isopropylbenzenesulfonamide;
N-[2-(1H-imidazol-1-yl)ethyl]-2-isopropyl-5-{[4-(methylamino)phenyl]sulfonyl}benzenesulfonamide;
5-({4-[(2-hydroxybutyl)amino]phenyl}sulfonyl)-N-[2-(1H-imidazol-1-yl)ethyl]-2-isopropylbenzenesulfonamide;

5-[(4-{[(2S)-1-(hydroxymethyl)-2-methylbutyl]amino }phenyl)sulfonyl]-N-[2-(1H-imidazol-1-yl)ethyl]-2-isopropylbenzenesulfonamide;
N-[3-(1H-imidazol-1-yl)propyl]-2-methyl-3-(phenylsulfonyl)benzenesulfonamide;
N-[3-(1H-Imidazol-1-yl)propyl]-5-(phenylsulfonyl)-2-propylbenzenesulfonamide;
5-[(4-Fluorophenyl)sulfonyl]-N-[3-(1H-imidazol-1-yl)propyl]-2-propylbenzenesulfonamide;
5-[(4-fluorophenyl)sulfonyl]-2-isopropyl-N-[2-(2-methyl-1H-imidazol-1-yl)ethyl]benzenesulfonamide;
N-[2-(2-ethyl-1H-imidazol-1-yl)ethyl]-5-[(4-fluorophenyl)sulfonyl]-2-isopropylbenzenesulfonamide;
5-[(4-fluorophenyl)sulfonyl]-2-isopropyl-N-[2-(2-isopropyl-1H-imidazol-1-yl)ethyl]benzenesulfonamide;
N-[3-(1H-Imidazol-1-yl)propyl]-2,4-diisopropyl-5-(phenylsulfonyl)benzenesulfonamide;
5-[(4-fluorophenyl)sulfonyl]-2-methyl-N-[2-(1-methylpyrrolidin-2-yl)ethyl]benzenesulfonamide;
5-[(4-fluorophenyl)sulfonyl]-2-methyl-N-(2-piperidin-1-ylethyl)benzenesulfonamide;
5-[(4-fluorophenyl)sulfonyl]-2-methyl-N-(2-morpholin-4-ylethyl)benzenesulfonamide;
5-[(4-fluorophenyl)sulfonyl]-2-methyl-N-[3-(2-oxopyrrolidin-1-yl)propyl]benzenesulfonamide;
N-[3-(dimethylamino)propyl]-5-[(4-fluorophenyl)sulfonyl]-2-methylbenzenesulfonamide;
5-[(4-fluorophenyl)sulfonyl]-N-(2-methoxyethyl)-2-methylbenzenesulfonamide;
5-[(4-fluorophenyl)sulfonyl]-2-methyl-N-(3-pyrrolidin-1-ylpropyl)benzenesulfonamide;
5-[(4-fluorophenyl)sulfonyl]-2-methyl-N-[3-(4-methylpiperazin-1-yl)propyl]benzenesulfonamide;
2-methyl-N-(3-morpholin-4-ylpropyl)-5-(phenylsulfonyl)benzenesulfonamide;
2-methyl-N-[2-(1-methylpyrrolidin-2-yl)ethyl]-5-(phenylsulfonyl)benzenesulfonamide;
2-methyl-5-(phenylsulfonyl)-N-(2-piperidin-1-ylethyl)benzenesulfonamide;
2-methyl-N-(2-morpholin-4-ylethyl)-5-(phenylsulfonyl)benzenesulfonamide;
2-methyl-N-[3-(2-oxopyrrolidin-1-yl)propyl]-5-(phenylsulfonyl)benzenesulfonamide;
2-methyl-5-(phenylsulfonyl)-N-(3-pyrrolidin-1-ylpropyl)benzenesulfonamide;
2-methyl-N-[3-(4-methylpiperazin-1-yl)propyl]-5-(phenylsulfonyl)benzenesulfonamide;
2-ethyl-N-(2-morpholin-4-ylethyl)-5-(phenylsulfonyl)benzenesulfonamide;
2-ethyl-5-(phenylsulfonyl)-N-(3-pyrrolidin-1-ylpropyl)benzenesulfonamide;
2-methoxy-N-(2-morpholin-4-ylethyl)-5-(phenylsulfonyl)benzenesulfonamide;
2-methoxy-5-(phenylsulfonyl)-N-(3-pyrrolidin-1-ylpropyl)benzenesulfonamide;
5-[(4-chlorophenyl)sulfonyl]-2-methyl-N-(2-morpholin-4-ylethyl)benzenesulfonamide;
2-methyl-5-(phenylsulfonyl)-N-tetrahydro-2H-pyran-4-ylbenzenesulfonamide;
2-methyl-5-(phenylsulfonyl)-N-(2-tetrahydro-2H-pyran-4-ylethyl)benzenesulfonamide;
2-ethyl-5-(phenylsulfonyl)-N-tetrahydro-2H-pyran-4-ylbenzenesulfonamide;
2-ethyl-5-(phenylsulfonyl)-N-(2-tetrahydro-2H-pyran-4-ylethyl)benzenesulfonamide;
5-[(4-fluorophenyl)sulfonyl]-2-methyl-N-tetrahydro-2H-pyran-4-ylbenzenesulfonamide;
5-[(4-fluorophenyl)sulfonyl]-2-methyl-N-(2-tetrahydro-2H-pyran-4-ylethyl)benzenesulfonamide;
5-[(4-fluorophenyl)sulfonyl]-2-isopropyl-N-tetrahydro-2H-pyran-4-ylbenzenesulfonamide;
5-[(4-fluorophenyl)sulfonyl]-2-isopropyl-N-(2-tetrahydro-2H-pyran-4-ylethyl)benzenesulfonamide;
2,4-dimethyl-5-(phenylsulfonyl)-N-tetrahydro-2H-pyran-4-ylbenzenesulfonamide;
2,4-dimethyl-5-(phenylsulfonyl)-N-(2-tetrahydro-2H-pyran-4-ylethyl)benzenesulfonamide;
5-[(4-fluorophenyl)sulfonyl]-2,4-dimethyl-N-(tetrahydro-2H-pyran-4-yl)benzenesulfonamide;
5-[(4-fluorophenyl)sulfonyl]-2,4-dimethyl-N-[2-(tetrahydro-2H-pyran-4-yl)ethyl]benzenesulfonamide;
2-chloro-N-(2-morpholin-4-ylethyl)-5-(phenylsulfonyl)benzenesulfonamide;
2,3-dimethyl-5-(phenylsulfonyl)-N-(tetrahydro-2H-pyran-4-yl)benzenesulfonamide;
2,3-dimethyl-5-(phenylsulfonyl)-N-[2-(tetrahydro-2H-pyran-4-yl)ethyl]benzenesulfonamide;
2-isopropyl-5-{[4-(methylamino)phenyl]sulfonyl)}-N-(tetrahydro-2H-pyran-4-yl)benzenesulfonamide;
2-isopropyl-5-{[4-(methylamino)phenyl]sulfonyl}-N-[2-(tetrahydro-2H-pyran-4-yl)ethyl]benzenesulfonamide;
2-chloro-5-(phenylsulfonyl)-N-(tetrahydro-2H-pyran-4-yl)benzenesulfonamide;
5-[(4-bromophenyl)sulfonyl]-2-methyl-N-(tetrahydro-2H-pyran-4-yl)benzenesulfonamide;
5-[(4-cyanophenyl)sulfonyl]-2-methyl-N-(tetrahydro-2H-pyran-4-yl)benzenesulfonamide;
N-[3-(1H-imidazol-1-yl)propyl]-2-methyl-5-(pyridin-2-ylsulfonyl)benzenesulfonamide;
5-[(2,4-dichlorophenyl)sulfonyl]-N-[3-(1H-imidazol-1-yl)propyl]-2-methylbenzenesulfonamide;
5-[(4-acetylphenyl)sulfonyl]-2-methyl-N-(tetrahydro-2H-pyran-4-yl)benzenesulfonamide;
5-[(4-bromophenyl)sulfonyl]-2-methyl-N-(2-pyridin-2-ylethyl)benzenesulfonamide;
2-methyl-5-(phenylsulfonyl)-N-(tetrahydro-2H-pyran-4-ylmethyl)benzenesulfonamide;
5-[(4-fluorophenyl)sulfonyl]-2-methyl-N-(tetrahydro-2H-pyran-4-ylmethyl)benzenesulfonamide;
2-ethyl-5-[(4-fluorophenyl)sulfonyl]-N-(tetrahydro-2H-pyran-4-ylmethyl)benzenesulfonamide;
2,4-dimethyl-5-(phenylsulfonyl)-N-(tetrahydro-2H-pyran-4-ylmethyl)benzenesulfonamide;
N-(2,2-dimethylpropyl)-2-methyl-5-(phenylsulfonyl)benzenesulfonamide;
5-({4-[(1E)-N-hydroxyethanimidoyl]phenyl}sulfonyl)-2-methyl-N-(tetrahydro-2H-pyran-4-yl)benzenesulfonamide;
5-[(4-acetylphenyl)sulfonyl]-2-methyl-N-(2-pyridin-2-ylethyl)benzenesulfonamide;
5-{[4-(1-hydroxy-1-methylethyl)phenyl]sulfonyl}-2-methyl-N-(2-pyridin-2-ylethyl)benzenesulfonamide;
2-methyl-5-(phenylsulfonyl)-N-(2,2,6,6-tetramethylpiperidin-4-yl)benzenesulfonamide;
N-(1-benzylpiperidin-4-yl)-2-methyl-5-(phenylsulfonyl)benzenesulfonamide;
N-(1-benzylpyrrolidin-3-yl)-2-methyl-5-(phenylsulfonyl)benzenesulfonamide;
ethyl 4-({[2-methyl-5-(phenylsulfonyl)phenyl]sulfonyl}amino)piperidine-1-carboxylate;
5-[(4-tert-butylphenyl)sulfonyl]-2-isopropyl-N-(tetrahydro-2H-pyran-4-yl)benzenesulfonamide;
5-[(4-tert-butylphenyl)sulfonyl]-2-methyl-N-(tetrahydro-2H-pyran-4-yl)benzenesulfonamide;
5-[(4-fluorophenyl)sulfonyl]-2-isopropyl-N-(tetrahydro-2H-pyran-4-ylmethyl)benzenesulfonamide;
tert-butyl 4-({[2-methyl-5-(phenylsulfonyl)phenyl]sulfonyl}amino)piperidine-1-carboxylate;
2-methyl-5-(phenylsulfonyl)-N-piperidin-4-ylbenzenesulfonamide;
2-methyl-5-(phenylsulfonyl)-N-[1-(phenylsulfonyl)piperidin-4-yl]benzenesulfonamide;
N-[1-(2-furoyl)piperidin-4-yl]-2-methyl-5-(phenylsulfonyl)benzenesulfonamide;
N-[1-(2-methoxybenzoyl)piperidin-4-yl]-2-methyl-5-(phenylsulfonyl)benzenesulfonamide;
N-[1-(3-methoxybenzoyl)piperidin-4-yl]-2-methyl-5-(phenylsulfonyl)benzenesulfonamide;
N-[1-(3,4-dimethoxybenzoyl)piperidin-4-yl]-2-methyl-5-(phenylsulfonyl)benzenesulfonamide;
2-methyl-5-(phenylsulfonyl)-N-{1-[3-(trifluoromethyl)benzoyl]piperidin-4-yl}benzenesulfonamide;
N-[1-(4-chlorobenzoyl)piperidin-4-yl]-2-methyl-5-(phenylsulfonyl)benzenesulfonamide;
N-[1-(4-methoxybenzoyl)piperidin-4-yl]-2-methyl-5-(phenylsulfonyl)benzenesulfonamide;
2-methyl-N-[1-(4-methylbenzoyl)piperidin-4-yl]-5-(phenylsulfonyl)benzenesulfonamide;
N-[1-(methoxyacetyl)piperidin-4-yl]-2-methyl-5-(phenylsulfonyl)benzenesulfonamide;
2-methyl-N-[1-(phenylacetyl)piperidin-4-yl]-5-(phenylsulfonyl)benzenesulfonamide;
N-[1-(cyclohexylcarbonyl)piperidin-4-yl]-2-methyl-5-(phenylsulfonyl)benzenesulfonamide;
2,6-dimethyl-3-(phenylsulfonyl)-N-(tetrahydro-2H-pyran-4-yl)benzenesulfonamide;
N-[1-(cyclopropylcarbonyl)piperidin-4-yl]-2-methyl-5-(phenylsulfonyl)benzenesulfonamide;
N-[1-(4-cyanobenzoyl)piperidin-4-yl]-2-methyl-5-(phenylsulfonyl)benzenesulfonamide;
N-[1-(3-cyanobenzoyl)piperidin-4-yl]-2-methyl-5-(phenylsulfonyl)benzenesulfonamide;
2-methyl-N-[1-(methylsulfonyl)piperidin-4-yl]-5-(phenylsulfonyl)benzenesulfonamide;
N-(1-acetylpiperidin-4-yl)-2-methyl-5-(phenylsulfonyl)benzenesulfonamide N-(4-{[4-({[2-methyl-5-(phenylsulfonyl)phenyl]sulfonyl}amino)piperidin-1-yl]carbonyl}phenyl)acetamide;
2-methyl-N-{1-[(1-methyl-1H-imidazol-4-yl)sulfonyl]piperidin-4-yl}-5-(phenylsulfonyl)benzenesulfonamide;
2-methyl-5-(phenylsulfonyl)-N-[1-(2-thienylsulfonyl)piperidin-4-yl]benzenesulfonamide;
N-(1-{[5-(dimethylamino)-1-naphthyl]sulfonyl}piperidin-4-yl)-2-methyl-5-(phenylsulfonyl)benzenesulfonamide;
N-[1-(1,3-benzodioxol-5-ylcarbonyl)piperidin-4-yl]-2-methyl-5-(phenylsulfonyl)benzenesulfonamide;
N-[1-(isoxazol-5-ylcarbonyl)piperidin-4-yl]-2-methyl-5-(phenylsulfonyl)benzenesulfonamide;
N-[1-(N,N-dimethylglycyl)piperidin-4-yl]-2-methyl-5-(phenylsulfonyl)benzenesulfonamide;
prop-2-yn-1-yl 4-({[2-methyl-5-(phenylsulfonyl)phenyl]sulfonyl}amino)piperidine-1-carboxylate;
methyl 4-({[2-methyl-5-(phenylsulfonyl)phenyl]sulfonyl}amino)piperidine-1-carboxylate;
2-methoxyphenyl 4-({[2-methyl-5-(phenylsulfonyl)phenyl]sulfonyl}amino)piperidine-1-carboxylate;
N-(tert-butyl)-4-({[2-methyl-5-(phenylsulfonyl)phenyl]sulfonyl}amino)piperidine-1-carboxamide;
N-cyclohexyl-4-({[2-methyl-5-(phenylsulfonyl)phenyl]sulfonyl}amino)piperidine-1-carboxamide;
2-methyl-N-(2-morpholin-4-ylethyl)-3-(phenylsulfonyl)benzenesulfonamide;
2-methyl-N-[1-(2-naphthoyl)piperidin-4-yl]-5-(phenylsulfonyl)benzenesulfonamide;
2-methyl-5-(phenylsulfonyl)-N-[1-(2-thienylcarbonyl)piperidin-4-yl]benzenesulfonamide;
isobutyl 4-({[2-methyl-5-(phenylsulfonyl)phenyl]sulfonyl}amino)piperidine-1-carboxylate;
N-{1-[4-(dimethylamino)benzoyl]piperidin-4-yl}-2-methyl-5-(phenylsulfonyl)benzenesulfonamide;
4-fluorophenyl 4-({[2-methyl-5-(phenylsulfonyl)phenyl]sulfonyl}amino)piperidine-1-carboxylate N-ethyl-4-({[2-methyl-5-(phenylsulfonyl)phenyl]sulfonyl}amino)piperidine-1-carboxamide;
2-methyl-N-[1-(morpholin-4-ylcarbonyl)piperidin-4-yl]-5-(phenylsulfonyl)benzenesulfonamide;
N,N-dimethyl-4-({[2-methyl-5-(phenylsulfonyl)phenyl]sulfonyl}amino)piperidine-1-carboxamide;
N-[1-(3,3-dimethylbutanoyl)piperidin-4-yl]-2-methyl-5-(phenylsulfonyl)benzenesulfonamide;
2-methyl-5-(phenylsulfonyl)-N-[1-(pyridin-3-ylcarbonyl)piperidin-4-yl]benzenesulfonamide;
tert-butyl 4-[({5-[(4-fluorophenyl)sulfonyl]-2-isopropylphenyl}sulfonyl)amino]piperidine-1-carboxylate;
N-(1-{5-(dimethylamino)-1-naphthyl]sulfonyl}piperidin-4-yl)-5-[(4-fluorophenyl)sulfonyl]-2-isopropylbenzenesulfonamide;
5-[(4-fluorophenyl)sulfonyl]-2-isopropyl-N-[1-(methoxyacetyl)piperidin-4-yl]benzenesulfonamide;
5-[(4-fluorophenyl)sulfonyl]-2-isopropyl-N-piperidin-4-ylbenzenesulfonamide;
N-(1-benzylpiperidin-4-yl)-5-[(4-fluorophenyl)sulfonyl)-2-isopropylbenzenesulfonamide;
ethyl 4-[({5-[(4-fluorophenyl)sulfonyl]-2-isopropylphenyl}sulfonyl)amino]piperidine-1-carboxylate;
5-[(4-fluorophenyl)sulfonyl]-2-isopropyl-N-(2-piperidin-1-ylethyl)benzenesulfonamide;
tert-butyl 4-{[(2-isopropyl-5-{[4-(methylamino)phenyl]sulfonyl}phenyl)sulfonyl]amino}piperidine-1-carboxylate;
N-(1-acetylpiperidin-4-yl)-5-[(4-fluorophenyl)sulfonyl]-2-isopropylbenzenesulfonamide;
N-[1-(cyclopropylcarbonyl)piperidin-4-yl]-5-[(4-fluorophenyl)sulfonyl]-2-isopropylbenzenesulfonamide;
N-[1-(4-cyanobenzoyl)piperidin-4-yl]-5-[(4-fluorophenyl)sulfonyl]-2-isopropylbenzenesulfonamide;
5-[(4-fluorophenyl)sulfonyl]-2-isopropyl-N-(tetrahydrofuran-2-ylmethyl)benzenesulfonamide;
5-({4-[(2-cyanoethyl)amino]phenyl}sulfonyl)-2-isopropyl-N-(tetrahydro-2H-pyran-4-yl)benzenesulfonamide;
5-({4-[(2-cyanoethyl)amino]phenyl}sulfonyl)-2-isopropyl-N-(tetrahydro-2H-pyran-4-ylmethyl)benzenesulfonamide;
5-({4-[(2-cyanoethyl)amino]phenyl}sulfonyl)-2-isopropyl-N-[2-(tetrahydro-2H-pyran-4-yl)ethyl]benzenesulfonamide;
N-(3′,6′-dihydroxy-3-oxo-3H-spiro[2-benzofuran-1,9′-xanthen]-5-yl)-4-[({5-[(4-fluorophenyl)sulfonyl]-2-isopropylphenyl}sulfonyl)amino]piperidine-1-carbothioamide;
2-isopropyl-5-[(2-methylphenyl)sulfonyl]-N-(tetrahydro-2H-pyran-4-yl)benzenesulfonamide;
5-[(3-chloro-2-methylphenyl)sulfonyl]-2-isopropyl-N-(tetrahydro-2H-pyran-4-yl)benzenesulfonamide;
2-isopropyl-5-{4-(methylamino)phenyl]sulfonyl}-N-(tetrahydro-2H-pyran-4-ylmethyl)benzenesulfonamide;
5-{4-(dimethylamino)phenyl]sulfonyl}-2-isopropyl-N-(tetrahydro-2H-pyran-4-yl)benzenesulfonamide;
5-{4-(dimethylamino)phenyl]sulfonyl}-2-isopropyl-N-(tetrahydro-2H-pyran-4-ylmethyl)benzenesulfonamide;
5-{4-(dimethylamino)phenyl]sulfonyl}-2-isopropyl-N-[2-(tetrahydro-2H-pyran-4-yl)ethyl]benzenesulfonamide;
2-isopropyl-5-{4-(4-methylpiperazin-1-yl)phenyl]sulfonyl}-N-(tetrahydro-2H-pyran-4-yl)benzenesulfonamide;
2-isopropyl-5-{4-(4-methylpiperazin-1-yl)phenyl]sulfonyl}-N-(tetrahydro-2H-pyran-4-ylmethyl)benzenesulfonamide;
2-isopropyl-5-{4-(4-methylpiperazin-1-yl)phenyl]sulfonyl}-N-[2-(tetrahydro-2H-pyran-4-yl)ethyl]benzenesulfonamide;
5-(phenylsulfonyl)-2-propyl-N-(tetrahydro-2H-pyran-4-yl)benzenesulfonamide;
5-(phenylsulfonyl)-2-propyl-N-(tetrahydro-2H-pyran-4-ylmethyl)benzenesulfonamide;
5-(phenylsulfonyl)-2-propyl-N-[2-(tetrahydro-2H-pyran-4-yl)ethyl]benzenesulfonamide;
tert-butyl 4-({[5-(phenylsulfonyl)-2-propylphenyl]sulfonyl}amino)piperidine-1-carboxylate;
5-[(4-fluorophenyl)sulfonyl]-2-propyl-N-(tetrahydro-2H-pyran-4-yl)benzenesulfonamide;
5-[(4-fluorophenyl)sulfonyl]-2-propyl-N-(tetrahydro-2H-pyran-4-ylmethyl)benzenesulfonamide;
5-[(4-fluorophenyl)sulfonyl]-2-propyl-N-[2-(tetrahydro-2H-pyran-4-yl)ethyl]benzenesulfonamide;
tert-Butyl 4-[({5-[(4-fluorophenyl)sulfonyl]-2-propylphenyl}sulfonyl)amino]piperidine-1-carboxylate;
2-isopropyl-5-{4-(methylamino)phenyl]sulfonyl}-N-piperidin-4-ylbenzenesulfonamide;
5-[(5-chloro-1,3-dimethyl-1H-pyrazol4-yl)sulfonyl]-2-isopropyl-N-(tetrahydro-2H-pyran-4-yl)benzenesulfonamide;
5-[(4-fluorophenyl)sulfonyl]-2-isopropyl-N-[1-(morpholin-4-ylcarbonyl)piperidin-4-yl]benzenesulfonamide;
4-[({5-[(4-fluorophenyl)sulfonyl]-2-isopropylphenyl}sulfonyl)amino]-N,N-dimethylpiperidine-1-carboxamide;
N-(1-benzylpiperidin-4-yl)-5-[(4-fluorophenyl)sulfonyl]-2-methylbenzenesulfonamide;
5-[(4-fluorophenyl)sulfonyl]-2-methyl-N-piperidin-4-ylbenzenesulfonamide;
5-({4-[(2-cyanoethyl)(methyl)amino]phenyl}sulfonyl)-2-isopropyl-N-(tetrahydro-2H-pyran-4-yl)benzenesulfonamide;
5-({4-[(2-cyanoethyl)(methyl)amino]phenyl}sulfonyl)-2-isopropyl-N-(tetrahydro-2H-pyran-4-ylmethyl)benzenesulfonamide;
N-(1-acetylpiperidin-4-yl)-5-[(4-fluorophenyl)sulfonyl]-2-methylbenzenesulfonamide;
N-[1-(4-cyanobenzoyl)piperidin-4-yl]-5-[(4-fluorophenyl)sulfonyl]-2-methylbenzenesulfonamide;
4-[({5-[(4-fluorophenyl)sulfonyl]-2-methylphenyl}sulfonyl)amino]-N,N-dimethylpiperidine-1-carboxamide;
5-[(4-fluorophenyl)sulfonyl]-N-[1-(methoxyacetyl)piperidin-4-yl]-2-methylbenzenesulfonamide;
2-isopropyl-5-[(3-methoxyphenyl)sulfonyl]-N-(tetrahydro-2H-pyran-4-yl)benzenesulfonamide;
5-{4-(dimethylamino)-2-methylphenyl]sulfonyl}-2-isopropyl-N-(2-pyridin-2-ylethyl)benzenesulfonamide;
2-(dimethylamino)-5-(phenylsulfonyl)-N-(2-pyridin-2-ylethyl)benzenesulfonamide;
5-[(4-fluorophenyl)sulfonyl]-2-methyl-N-[1-(morpholin-4-ylcarbonyl)piperidin-4-yl]benzenesulfonamide;
2-chloro-5-[(3-methylphenyl)sulfonyl]-N-(tetrahydro-2H-pyran-4-yl)benzenesulfonamide;
2-chloro-5-[(3-methoxyphenyl)sulfonyl]-N-(tetrahydro-2H-pyran-4-yl)benzenesulfonamide;
2-chloro-5-[(1-methyl-1H-indol-5-yl)sulfonyl]-N-(tetrahydro-2H-pyran-4-yl)benzenesulfonamide;
5-(phenylsulfonyl)-N-(tetrahydro-2H-pyran-4-yl)-2-(trifluoromethyl)benzenesulfonamide;
2,4-diisopropyl-5-(phenylsulfonyl)-N-(tetrahydro-2H-pyran-4-yl)benzenesulfonamide;
2,4-diisopropyl-5-(phenylsulfonyl)-N-(tetrahydro-2H-pyran-4-ylmethyl)benzenesulfonamide;
2,4-diisopropyl-5-(phenylsulfonyl)-N-[2-(tetrahydro-2H-pyran-4-yl)ethyl]benzenesulfonamide;
5-[(4-Fluorophenyl)sulfonyl]-2,4-diisopropyl-N-(tetrahydro-2H-pyran-4-yl)benzenesulfonamide;
2-chloro-5-[(4-fluorophenyl)sulfonyl]-N-(tetrahydro-2H-pyran-4-yl)benzenesulfonamide;
tert-butyl 4-({[2-chloro-5-(phenylsulfonyl)phenyl]sulfonyl}amino)piperidine-1-carboxylate 2-chloro-5-(phenylsulfonyl)-N-piperidin-4-ylbenzenesulfonamide;
tert-butyl 4-[4-({4-isopropyl-3-[(tetrahydro-2H-pyran-4-ylamino)sulfonyl]phenyl}sulfonyl)phenyl]piperazine-1-carboxylate 5-({4-cis-3,5-dimethylpiperazin-1-ylphenyl}sulfonyl)-2-isopropyl-N-(tetrahydro-2H-pyran-4-yl)benzenesulfonamide;
5-({4-trans-2,5-dimethylpiperazin-1-ylphenyl}sulfonyl)-2-isopropyl-N-(tetrahydro-2H-pyran-4-yl)benzenesulfonamide;
2-isopropyl-5-[(4-piperazin-1-ylphenyl)sulfonyl]-N-(tetrahydro-2H-pyran-4-yl)benzenesulfonamide;
5-[(4-Fluorophenyl)sulfonyl]-2,4-diisopropyl-N-(tetrahydro-2H-pyran-4-ylmethyl)benzenesulfonamide;
5-[(4-Fluorophenyl)sulfonyl]-2,4-diisopropyl-N-[2-(tetrahydro-2H-pyran-4-yl)ethyl]benzenesulfonamide;
2-chloro-N-[3-(4-methylpiperazin-1-yl)propyl]-5-(phenylsulfonyl)benzenesulfonamide;
1-{2-chloro-5-(phenylsulfonyl)phenyl]sulfonyl}-N,N-diethylpyrrolidin-3-amine;
ethyl 1-{2-chloro-5-(phenylsulfonyl)phenyl]sulfonyl}piperidine-3-carboxylate;
2-chloro-5-(phenylsulfonyl)-N-[1-(trifluoroacetyl)piperidin-4-yl]benzenesulfonamide;
2-chloro-N-[1-(2,2-dimethylpropanoyl)piperidin-4-yl]-5-(phenylsulfonyl)benzenesulfonamide;
N-(tert-butyl)-4-({[2-chloro-5-(phenylsulfonyl)phenyl]sulfonyl}amino)piperidine-1-carboxamide;
2-chloro-N-[1-(morpholin-4-ylcarbonyl)piperidin-4-yl]-5-(phenylsulfonyl)benzenesulfonamide;
2-chloro-N-(1-cyanopiperidin-4-yl)-5-(phenylsulfonyl)benzenesulfonamide;
N³-({5-[(4-fluorophenyl)sulfonyl]-2-methylphenyl}sulfonyl)-beta-alaninamide;
methyl N-({5-[(4-fluorophenyl)sulfonyl]-2-methylphenyl}sulfonyl)-beta-alaninate;
N-(2-cyanoethyl)-5-[(4-fluorophenyl)sulfonyl]-2-methylbenzenesulfonamide;
N-{2-[({5-[(4-fluorophenyl)sulfonyl]-2-methylphenyl}sulfonyl)amino]ethyl}acetamide;
N-[2-(diethylamino)ethyl]-5-[(4-fluorophenyl)sulfonyl]-2-methylbenzenesulfonamide;
N-[2-(dimethylamino)ethyl]-5-[(4-fluorophenyl)sulfonyl]-2-methylbenzenesulfonamide;
5-[(4-fluorophenyl)sulfonyl]-N-(3-methoxypropyl)-2-methylbenzenesulfonamide;
N-[3-(diethylamino)propyl]-5-[(4-fluorophenyl)sulfonyl]-2-methylbenzenesulfonamide;
N-({5-[(4-fluorophenyl)sulfonyl]-2-methylphenyl}sulfonyl)-beta-alanine;
N³-{[2-methyl-5-(phenylsulfonyl)phenyl]sulfonyl}-beta-alaninamide;
methyl N-{2-methyl-5-(phenylsulfonyl)phenyl]sulfonyl}-beta-alaninate;
N-[2-(diethylamino)ethyl]-2-methyl-5-(phenylsulfonyl)benzenesulfonamide;
N-[2-(dimethylamino)ethyl]-2-methyl-5-(phenylsulfonyl)benzenesulfonamide;
N-(3-methoxypropyl)-2-methyl-5-(phenylsulfonyl)benzenesulfonamide;
N-[3-(dimethylamino)propyl]-2-methyl-5-(phenylsulfonyl)benzenesulfonamide;
N-(2-methoxyethyl)-2-methyl-5-(phenylsulfonyl)benzenesulfonamide;
N-[3-(diethylamino)propyl]-2-methyl-5-(phenylsulfonyl)benzenesulfonamide;
methyl N-{[2-ethyl-5-(phenylsulfonyl)phenyl]sulfonyl}-beta-alaninate;
2-ethyl-N-(3-methoxypropyl)-5-(phenylsulfonyl)benzenesulfonamide;
N-[3-(dimethylamino)propyl]-2-ethyl-5-(phenylsulfonyl)benzenesulfonamide;
2-ethyl-N-(2-methoxyethyl)-5-(phenylsulfonyl)benzenesulfonamide;
N-[3-(diethylamino) propyl]-2-ethyl-5-(phenylsulfonyl)benzenesulfonamide;
methyl N-{2-methoxy-5-(phenylsulfonyl)phenyl]sulfonyl)}-beta-alaninate;
2-methoxy-N-(3-methoxypropyl)-5-(phenylsulfonyl)benzenesulfonamide;
N-[3-(dimethylamino)propyl]-2-methoxy-5-(phenylsulfonyl)benzenesulfonamide;
2-methoxy-N-(2-methoxyethyl)-5-(phenylsulfonyl)benzenesulfonamide;
N-[3-(diethylamino)propyl]-2-methoxy-5-(phenylsulfonyl)benzenesulfonamide;
2-methyl-5-(phenylsulfonyl)-N-propylbenzenesulfonamide;
N-(1-ethylpropyl)-2-methyl-5-(phenylsulfonyl)benzenesulfonamide;
N-cyclobutyl-2-methyl-5-(phenylsulfonyl)benzenesulfonamide;
N-cyclopentyl-2-methyl-5-(phenylsulfonyl)benzenesulfonamide;
N-cyclohexyl-2-methyl-5-(phenylsulfonyl)benzenesulfonamide;
2-methyl-5-(phenylsulfonyl)-N-(2,2,2-trifluoroethyl)benzenesulfonamide;
N-(2-hydroxy-1,1-dimethylethyl)-2-methyl-5-(phenylsulfonyl)benzenesulfonamide;
N-cyclopropyl-2-methyl-5-(phenylsulfonyl)benzenesulfonamide;
N-cyclopropyl-5-[(4-fluorophenyl)sulfonyl]-2-isopropylbenzenesulfonamide;
N-cyclobutyl-5-[(4-fluorophenyl)sulfonyl]-2-isopropylbenzenesulfonamide;
N-cyclopentyl-5-[(4-fluorophenyl)sulfonyl]-2-isopropylbenzenesulfonamide;
N-cyclohexyl-5-[(4-fluorophenyl)sulfonyl]-2-isopropylbenzenesulfonamide;
N-cyclopentyl-5-(phenylsulfonyl)-2-propylbenzenesulfonamide;
N-cyclopentyl-5-[(4-fluorophenyl)sulfonyl]-2-propylbenzenesulfonamide;
N-cyclopentyl-2,4-diisopropyl-5-(phenylsulfonyl)benzenesulfonamide;
N-cyclopentyl-5-[(4-fluorophenyl)sulfonyl]-2,4-diisopropylbenzenesulfonamide;
2-chloro-N-(2-cyanoethyl)-5-(phenylsulfonyl)benzenesulfonamide;
methyl N-{[2-chloro-5-(phenylsulfonyl)phenyl]sulfonyl}-2-methylalaninate;
N-[2-(3,4-dimethoxyphenyl)ethyl]-N,2-dimethyl-5-(phenylsulfonyl)benzenesulfonamide;
N-allyl-N-[2-(3,4-dimethoxyphenyl)ethyl]-2-methyl-5-(phenylsulfonyl)benzenesulfonamide;
N-[2-(3,4-dimethoxyphenyl)ethyl]-2-methyl-5-(phenylsulfonyl)-N-prop-2-ynylbenzenesulfonamide;
N-[2-(2-fluorophenyl)ethyl]-N,2-dimethyl-5-[(4-methylphenyl)sulfonyl]benzenesulfonamide;
N-allyl-N-[2-(2-fluorophenyl)ethyl]-2-methyl-5-[(4-methylphenyl)sulfonyl]benzene sulfonamide;
N-[2-(2-fluorophenyl)ethyl]-2-methyl-5-[(4-methylphenyl)sulfonyl]-N-prop-2-ynylbenzenesulfonamide;
N,2-dimethyl-N-(2-phenylethyl)-5-(phenylsulfonyl)benzenesulfonamide;
1-{[2-methyl-5-(phenylsulfonyl)phenyl]sulfonyl}-4-(2-oxo-2-pyrrolidin-1-ylethyl)piperazine;
N,N-diethyl-N-[2-(4-{2-methyl-5-(phenylsulfonyl)phenyl]sulfonyl}piperazin-1-yl)ethyl]amine;
4-[2-(4-{2-methyl-5-(phenylsulfonyl)phenyl]sulfonyl}piperazin-1-yl)ethyl]morpholine;
1-{[2-methyl-5-(phenylsulfonyl)phenyl]sulfonyl}-4-(2-pyrrolidin-1-ylethyl)piperazine;
4-[3-(4-{2-methyl-5-(phenylsulfonyl)phenyl]sulfonyl}piperazin-1-yl)propyl]morpholine;
2-ethyl-N-methyl-N-(2-phenylethyl)-5-(phenylsulfonyl)benzenesulfonamide;
1-{[2-ethyl-5-(phenylsulfonyl)phenyl]sulfonyl}-4-(2-oxo-2-pyrrolidin-1-ylethyl)piperazine;
N,N-diethyl-N-[2-(4-{2-ethyl-5-(phenylsulfonyl)phenyl]sulfonyl}piperazin-1-yl)ethyl]amine;
4-[2-(4-{2-ethyl-5-(phenylsulfonyl)phenyl]sulfonyl}piperazin-1-yl)ethyl]morpholine;
1-{[2-ethyl-5-(phenylsulfonyl)phenyl]sulfonyl}-4-(2-pyrrolidin-1-ylethyl)piperazine;
4-[3-(4-{2-ethyl-5-(phenylsulfonyl)phenyl]sulfonyl}piperazin-1-yl)propyl]morpholine;
N-[1-(cyclopropylcarbonyl)piperidin-4-yl]-N-({5-[(4-fluorophenyl)sulfonyl]-2-methylphenyl}sulfonyl)cyclopropanecarboxamide;
1-{[2-chloro-5-(phenylsulfonyl)phenyl]sulfonyl}-4-pyrrolidin-1-ylpiperidine;
4-[2-(4-{[2-chloro-5-(phenylsulfonyl)phenyl]sulfonyl}piperazin-1-yl)ethyl]morpholine;
1-(1,3-benzodioxol-5-ylmethyl)-4-{2-chloro-5-(phenylsulfonyl)phenyl]sulfonyl )piperazine;
tert-butyl (1-{[2-chloro-5-(phenylsulfonyl)phenyl]sulfonyl}pyrrolidin-3-yl)carbamate;
tert-butyl (1-{[2-chloro-5-(phenylsulfonyl)phenyl]sulfonyl}piperidin-4-yl)carbamate;
2-chloro-N-methyl-5-(phenylsulfonyl)-N-(2-pyridin-2-ylethyl)benzenesulfonamide;
1-{[2-chloro-5-(phenylsulfonyl)phenyl]sulfonyl}-4-[(2,5-dimethyl-1H-pyrrol-1-yl)methyl]piperidine;
2-chloro-N-(2-hydroxy-1,1-dimethylethyl)-5-(phenylsulfonyl)benzenesulfonamide;
2-chloro-N-(cyanomethyl)-5-(phenylsulfonyl)benzenesulfonamide;
N-(2-cyanoethyl)-5-[(4-fluorophenyl)sulfonyl]-2-isopropylbenzenesulfonamide;
2-methyl-N-(3-oxo-3-pyrrolidin-1-ylpropyl)-5-(phenylsulfonyl)benzenesulfonamide;
N-(tert-butyl)-N³-{[2-methyl-5-(phenylsulfonyl)phenyl]sulfonyl}-β-alaninamide;
N³-{2-methyl-5-(phenylsulfonyl)phenyl]sulfonyl}-N-(1,2,3,4-tetrahydronaphthalen-1-yl)-β-alaninamide;
N-methyl-N³-{2-methyl-5-(phenylsulfonyl)phenyl]sulfonyl}-N-phenyl-p-alaninamide;
2-methyl-N-[3-(6-methyl-3,4-dihydroquinolin-1(2H)-yl)-3-oxopropyl]-5-(phenylsulfonyl)benzenesulfonamide;
2-chloro-N-(2-hydroxyethyl)-5-(phenylsulfonyl)benzenesulfonamide;
2-chloro-N-(2-hydroxy-1-methylethyl)-5-(phenylsulfonyl)benzenesulfonamide;
2-chloro-N-[2-hydroxy-1-(hydroxymethyl)ethyl]-5-(phenylsulfonyl)benzenesulfonamide;
N-(2-cyanoethyl)-5-(phenylsulfonyl)-2-(trifluoromethyl)benzenesulfonamide;
N-(2-hydroxyethyl)-5-(phenylsulfonyl)-2-(trifluoromethyl)benzenesulfonamide;
N-(2-hydroxy-1-methylethyl)-5-(phenylsulfonyl)-2-(trifluoromethyl)benzenesulfonamide;
N-[(1S)-2-hydroxy-1-methylethyl]-5-(phenylsulfonyl)-2-(trifluoromethyl)benzenesulfonamide;
N-[(1R)-2-hydroxy-1-methylethyl]-5-(phenylsulfonyl)-2-(trifluoromethyl)benzenesulfonamide;
5-[(4-fluorophenyl)sulfonyl]-N-(2-hydroxy-1-methylethyl)-2-isopropylbenzenesulfonamide;
5-[(4-fluorophenyl)sulfonyl]-N-[1-(hydroxymethyl)-2-methylpropyl]-2-isopropylbenzenesulfonamide;
5-[(4-fluorophenyl)sulfonyl]-N-(2-hydroxybutyl)-2-isopropylbenzenesulfonamide;
N-(2-cyanoethyl)-5-[(3-methoxyphenyl)sulfonyl]-2-(trifluoromethyl)benzenesulfonamide;
5-[(3-chlorophenyl)sulfonyl]-N-(2-cyanoethyl)-2-(trifluoromethyl)benzenesulfonamide;
5-(phenylsulfonyl)-N-(tetrahydro-2H-pyran-4-ylmethyl)-2-(trifluoromethyl)benzenesulfonamide;
N-(2-morpholin-4-ylethyl)-5-(phenylsulfonyl)-2-(trifluoromethyl)benzenesulfonamide;
N-(3-morpholin-4-ylpropyl)-5-(phenylsulfonyl)-2-(trifluoromethyl)benzenesulfonamide;
N-(3-methoxypropyl)-5-(phenylsulfonyl)-2-(trifluoromethyl)benzenesulfonamide;
N-[1-(hydroxymethyl)-2-methylpropyl]-5-(phenylsulfonyl)-2-(trifluoromethyl)benzenesulfonamide;
N-[(1R)-1-(hydroxymethyl)propyl]-5-(phenylsulfonyl)-2-(trifluoromethyl)benzenesulfonamide;
N-(2-hydroxyethyl)-5-[(3-methoxyphenyl)sulfonyl]-2-(trifluoromethyl)benzenesulfonamide;
N-(2-cyanoethyl)-5-[(4-methoxyphenyl)sulfonyl]-2-(trifluoromethyl)benzenesulfonamide;
N-(2-cyanoethyl)-5-[(4-hydroxyphenyl)sulfonyl]-2-(trifluoromethyl)benzenesulfonamide;
N-[(1-hydroxycyclohexyl)methyl]-5-(phenylsulfonyl)-2-(trifluoromethyl)benzenesulfonamide;
5-[(4-fluorophenyl)sulfonyl]-2-isopropyl-N-(2-piperidin-3-ylethyl)benzenesulfonamide;
N-{[5-(phenylsulfonyl)-2-(trifluoromethyl)phenyl]sulfonyl)}-β-alanine;
4-({[5-(phenylsulfonyl)-2-(trifluoromethyl)phenyl]sulfonyl}amino)butanoic acid;
tert-butyl 4-[({[5-(phenylsulfonyl)-2-(trifluoromethyl)phenyl]sulfonyl}amino)methyl]piperidine-1-carboxyate;
5-(phenylsulfonyl)-N-(piperidin-4-ylmethyl)-2-(trifluoromethyl)benzenesulfonamide;
tert-butyl [trans-4-({[5-(phenylsulfonyl)-2-(trifluoromethyl)phenyl]sulfonyl}amino)cyclohexyl]carbamate;
4-oxo-4-{4-[({[5-(phenylsulfonyl)-2-(trifluoromethyl)phenyl]sulfonyl}amino)methyl]piperidin-1-yl}butanoic acid;
5-oxo-5-{4-[({[5-(phenylsulfonyl)-2-(trifluoromethyl)phenyl]sulfonyl}amino)methyl]piperidin-1-yl pentanoic acid;
3-{(4-[({[5-(phenylsulfonyl)-2-(trifluoromethyl)phenyl]sulfonyl}amino)methyl]piperidin-1-ylsulfonyl)benzoic acid;
tert-butyl 2-[2-({[5-(phenylsulfonyl)-2-(trifluoromethyl)phenyl]sulfonyl}amino)ethyl]pyrrolidine-1-carboxylate;
5-(phenylsulfonyl)-N-(2-pyrrolidin-2-ylethyl)-2-(trifluoromethyl)benzenesulfonamide;
{2-[2-({[5-(phenylsulfonyl)-2-(trifluoromethyl)phenyl]sulfonyl}amino)ethyl]pyrrolidin-1-yl}acetic acid;
4-oxo-4-{2-[2-({[5-(phenylsulfonyl)-2-(trifluoromethyl)phenyl]sulfonyl}amino)ethyl]pyrrolidin-1-yl}butanoic acid;
3-({2-[2-({[5-(phenylsulfonyl)-2-(trifluoromethyl)phenyl]sulfonyl}amino)ethyl]pyrrolidin-1-yl}sulfonyl)benzoic acid;
tert-butyl 4-[2-({[5-(phenylsulfonyl)-2-(trifluoromethyl)phenyl]sulfonyl}amino)ethyl]piperidine-1-carboxylate;
methyl 3-({[2-isopropyl-5-(phenylsulfonyl)phenyl]sulfonyl}amino)cyclohexanecarboxylate;
methyl 4-({[2-isopropyl-5-(phenylsulfonyl)phenyl]sulfonyl}amino)cyclohexanecarboxylate;
methyl trans-4-[({[2-isopropyl-5-(phenylsulfonyl)phenyl]sulfonyl}amino)methyl]cyclohexanecarboxylate;
5-(phenylsulfonyl)-N-(2-piperidin-4-ylethyl)-2-(trifluoromethyl)benzenesulfonamide;
{4-[({[5-(phenylsulfonyl)-2-(trifluoromethyl)phenyl]sulfonyl}amino)methyl]piperidin-1-yl}acetic acid;
{4-[2-({[5-(phenylsulfonyl)-2-(trifluoromethyl)phenyl]sulfonyl}amino)ethyl]piperidin-1-yl}acetic acid;
methyl 3-({[5-(phenylsulfonyl)-2-(trifluoromethyl)phenyl]sulfonyl}amino)cyclohexanecarboxylate;
methyl 4-({[5-(phenylsulfonyl)-2-(trifluoromethyl)phenyl]sulfonyl}amino)cyclohexanecarboxylate;
methyl trans-4-[({[5-(phenylsulfonyl)-2-(trifluoromethyl)phenyl]sulfonyl}amino)methyl]cyclohexanecarboxylate;
3-({[2-isopropyl-5-(phenylsulfonyl)phenyl]sulfonyl}amino)cyclohexanecarboxylic acid;
4-({[2-isopropyl-5-(phenylsulfonyl)phenyl]sulfonyl}amino)cyclohexanecarboxylic acid;
trans-4-[({[2-isopropyl-5-(phenylsulfonyl)phenyl]sulfonyl}amino)methyl]cyclohexanecarboxylic acid;
3-{4-[({[5-(phenylsulfonyl)-2-(trifluoromethyl)phenyl]sulfonyl}amino)methyl]piperidin-1-yl}benzoic acid;
3-{4-[2-({[5-(phenylsulfonyl)-2-(trifluoromethyl)phenyl]sulfonyl}amino)ethyl]piperidin-1-yl}benzoic acid;
N-(trans-4-aminocyclohexyl)-5-(phenylsulfonyl)-2-(trifluoromethyl)benzenesulfonamide;
3-({[5-(phenylsulfonyl)-2-(trifluoromethyl)phenyl]sulfonyl}amino)cyclohexanecarboxylic acid;
4-({[5-(phenylsulfonyl)-2-(trifluoromethyl)phenyl]sulfonyl}amino)cyclohexanecarboxylic acid;
trans-4-[({[5-(phenylsulfonyl)-2-(trifluoromethyl)phenyl]sulfonyl}amino)methyl]cyclohexanecarboxylic acid;
N-(trans-4-hydroxycyclohexyl)-5-(phenylsulfonyl)-2-(trifluoromethyl)benzenesulfonamide;
tert-butyl 4-[({[5-(phenylsulfonyl)-2-(trifluoromethyl)phenyl]sulfonyl}amino)carbonyl]piperidine-1-carboxylate;
N-{5-(phenylsulfonyl)-2-(trifluoromethyl)phenyl]sulfonyl piperidine-4-carboxamide;
5-(phenylsulfonyl)-N-(tetrahydro-2H-pyran-4-yl)-2-(trifluoromethoxy)benzenesulfonamide;
2-isopropyl-5-(phenylsulfonyl)-N-(tetrahydro-2H-pyran-4-yl)benzenesulfonamide;
5-(phenylsulfonyl)-N-piperidin-4-yl-2-(trifluoromethyl)benzenesulfonamide;
2-Methoxy-5-(phenylsulfonyl)-N-(tetrahydro-2H-pyran-4-yl)benzenesulfonamide;
5-[(3-methoxyphenyl)sulfonyl]-N-(tetrahydro-2H-pyran-4-yl)-2-(trifluoromethyl)benzenesulfonamide;
5-[(3-hydroxyphenyl)sulfonyl]-N-(tetrahydro-2H-pyran-4-yl)-2-(trifluoromethyl)benzenesulfonamide;
5-[(3-chlorophenyl)sulfonyl]-N-(tetrahydro-2H-pyran-4-yl)-2-(trifluoromethyl)benzenesulfonamide;
5-(phenylsulfonyl)-N-[(3S)-piperidin-3-yl]-2-(trifluoromethyl)benzenesulfonamide;
5-(phenylsulfonyl)-N-[(3R)-piperidin-3-yl]-2-(trifluoromethyl)benzenesulfonamide;
5-[(1,2-dimethyl-1H-indol-5-yl)sulfonyl]-N-piperidin-4-yl-2-(trifluoromethyl)benzenesulfonamide;
5-(phenylsulfonyl)-N-[(3S)-pyrrolidin-3-yl]-2-(trifluoromethyl)benzenesulfonamide;
5-(phenylsulfonyl)-N-[(3R)-pyrrolidin-3-yl]-2-(trifluoromethyl)benzenesulfonamide;
2-methyl-5-(phenylsulfonyl)-N-(tetrahydro-2H-thiopyran-4-yl)benzenesulfonamide;
5-[(4-fluorophenyl)sulfonyl]-2-isopropyl-N-(tetrahydro-2H-thiopyran-4-yl)benzenesulfonamide;
5-[(4-fluorophenyl)sulfonyl]-N-(tetrahydro-2H-pyran-4-yl)-2-(trifluoromethyl)benzenesulfonamide;
5-[(2-fluorophenyl)sulfonyl]-N-(tetrahydro-2H-pyran-4-yl)-2-(trifluoromethyl)benzenesulfonamide;
5-(phenylsulfonyl)-N-pyrrolidin-3-yl-2-(trifluoromethyl)benzenesulfonamide;
N-[(2R*,4S*,6S*)-2,6-dimethyltetrahydro-2H-pyran-4-y]-5-[(4-fluorophenyl)sulfonyl]-2-methylbenzenesulfonamide;
5-{[4-(methylamino)phenyl]sulfonyl}-N-piperidin-4-yl-2-(trifluoromethyl)benzenesulfonamide;
5-{[4-(dimethylamino)phenyl]sulfonyl}-N-piperidin-4-yl-2-(trifluoromethyl)benzenesulfonamide;
5-({4-[(2-hydroxyethyl)amino]phenyl}sulfonyl)-N-piperidin-4-yl-2-(trifluoromethyl)benzenesulfonamide;
N-(1,1-dioxidotetrahydro-2H-thiopyran-4-yl)-5-[(4-fluorophenyl)sulfonyl]-2-isopropylbenzenesulfonamide;
5-{2-(methylamino)phenyl]sulfonyl}-N-piperidin-4-yl-2-(trifluoromethyl)benzenesulfonamide;
5-{2-(dimethylamino)phenyl]sulfonyl}-N-piperidin-4-yl-2-(trifluoromethyl)benzenesulfonamide;
5-({2-[(2-hydroxyethyl)amino]phenyl}sulfonyl)-N-piperidin-4-yl-2-(trifluoromethyl)benzenesulfonamide;
2-ethyl-5-(phenylsulfonyl)-N-piperidin-4-ylbenzenesulfonamide;
2,3-dimethyl-5-(phenylsulfonyl)-N-piperidin-4-ylbenzenesulfonamide;
2,4-dimethyl-5-(phenylsulfonyl)-N-piperidin-4-ylbenzenesulfonamide;
2-isopropyl-5-(phenylsulfonyl)-N-piperidin-4-ylbenzenesulfonamide;
5-(phenylsulfonyl)-N-piperidin-4-yl-2-propylbenzenesulfonamide;
2-isopropyl-5-(phenylsulfonyl)-N-(2,2,6,6-tetramethylpiperidin-4-yl)benzenesulfonamide;
5-(phenylsulfonyl)-N-(2,2,6,6-tetramethylpiperidin-4-yl)-2-(trifluoromethyl)benzenesulfonamide;
5-[(4-methoxyphenyl)sulfonyl]-N-piperidin-4-yl-2-(trifluoromethyl)benzenesulfonamide;
5-[(3-bromophenyl)sulfonyl]-N-piperidin-4-yl-2-(trifluoromethyl)benzenesulfonamide;
2-chloro-N-[1-(4-fluorobenzoyl)piperidin-4-yl]-5-(phenylsulfonyl)benzenesulfonamide;
2-chloro-N-[1-(4-cyanobenzoyl)piperidin-4-yl]-5-(phenylsulfonyl)benzenesulfonamide;
2-chloro-5-(phenylsulfonyl)-N-{1-[4-(trifluoromethyl)benzoyl]piperidin-4-yl}benzenesulfonamide;
5-(phenylsulfonyl)-2-(trifluoromethyl)-N-{1-[4-(trifluoromethyl)benzoyl]piperidin-4-yl}benzenesulfonamide;
N-[1-(2-chlorobenzoyl)piperidin-4-yl]-5-(phenylsulfonyl)-2-(trifluoromethyl)benzenesulfonamide;
N-[1-(2-methoxybenzoyl)piperidin-4-yl]-5-(phenylsulfonyl)-2-(trifluoromethyl)benzenesulfonamide;
N-[1-(3-chlorobenzoyl)piperidin-4-yl]-5-(phenylsulfonyl)-2-(trifluoromethyl)benzenesulfonamide;
N-[1-(3,4-difluorobenzoyl)piperidin-4-yl]-5-(phenylsulfonyl)-2-(trifluoromethyl)benzenesulfonamide;
N-[1-(3,5-difluorobenzoyl)piperidin-4-yl]-5-(phenylsulfonyl)-2-(trifluoromethyl)benzenesulfonamide;
N-[1-(2,6-dimethoxybenzoyl)piperidin-4-yl]-5-(phenylsulfonyl)-2-(trifluoromethyl)benzenesulfonamide;
N-[1-(2,4-dimethoxybenzoyl)piperidin-4-yl]-5-(phenylsulfonyl)-2-(trifluoromethyl)benzenesulfonamide;
5-(phenylsulfonyl)-N-{1-[4-(trifluoromethoxy)benzoyl]piperidin-4-yl}-2-(trifluoromethyl)benzenesulfonamide;
N-{1-[2-fluoro-4-(trifluoromethyl)benzoyl]piperidin-4-yl}-5-(phenylsulfonyl)-2-(trifluoromethyl)benzenesulfonamide;
N-{1-[3-fluoro4-(trifluoromethyl)benzoyl]piperidin-4-yl}-5-(phenylsulfonyl)-2-(trifluoromethyl)benzenesulfonamide;
N-[1-(3,4-dichlorobenzoyl)piperidin-4-yl]-5-(phenylsulfonyl)-2-(trifluoromethyl)benzenesulfonamide;
N-[1-(4-chlorobenzoyl)piperidin-4-yl]-5-(phenylsulfonyl)-2-(trifluoromethyl)benzenesulfonamide;
N-(1-isonicotinoylpiperidin-4-yl)-5-(phenylsulfonyl)-2-(trifluoromethyl)benzenesulfonamide;
N-[1-(2-chloro-6-methoxyisonicotinoyl)piperidin-4-yl]-5-(phenylsulfonyl)-2-(trifluoromethyl)benzenesulfonamide;
N-[1-(2-chloro-4-fluorobenzoyl)piperidin-4-yl]-5-(phenylsulfonyl)-2-(trifluoromethyl)benzenesulfonamide;
5-(phenylsulfonyl)-N-[1-(2,4,6-trifluorobenzoyl)piperidin-4-yl]-2-(trifluoromethyl)benzenesulfonamide;
N-[1-(4-tert-butylbenzoyl)piperidin-4-yl]-5-(phenylsulfonyl)-2-(trifluoromethyl)benzenesulfonamide;
N-(4-{4-({[5-(phenylsulfonyl)-2-(trifluoromethyl)phenyl]sulfonyl}amino)piperidin-1-yl]carbonyl}phenyl)acetamide;
5-(phenylsulfonyl)-2-(trifluoromethyl)-N-(1-{4-(trifluoromethyl)phenyl]carbonothioyl}piperidin-4-yl)benzenesulfonamide;
N-[1-(4-tert-butylbenzoyl)piperidin-4-yl]-5-[(3-methoxyphenyl)sulfonyl]-2-(trifluoromethyl)benzenesulfonamide;
N-[1-(4-tert-butylbenzoyl)piperidin-4-yl]-5-[(3-chlorophenyl)sulfonyl]-2-(trifluoromethyl)benzenesulfonamide;
5-(phenylsulfonyl)-N-{1-[2-(trifluoromethoxy)benzoyl]piperidin-4-yl}-2-(trifluoromethyl)benzenesulfonamide;
N-(1-benzoylpiperidin-4-yl)-5-(phenylsulfonyl)-2-(trifluoromethyl)benzenesulfonamide;
N-[1-(4-tert-butylbenzoyl)pyrrolidin-3-yl]-5-(phenylsulfonyl)-2-(trifluoromethyl)benzenesulfonamide;
N-[1-(4-tert-butylbenzoyl)piperidin-4-yl]-5-[(3-hydroxyphenyl)sulfonyl]-2-(trifluoromethyl)benzenesulfonamide;
N-[1-(4-benzoylbenzoyl)piperidin-4-yl]-5-(phenylsulfonyl)-2-(trifluoromethyl)benzenesulfonamide;
N-[1-(3-benzoylbenzoyl)piperidin-4-yl]-5-(phenylsulfonyl)-2-(trifluoromethyl)benzenesulfonamide;
1-Isopropyl-4-(phenylsulfonyl)benzene;
2-Isopropyl-N-[1-(2-methoxybenzoyl)piperidin-4-yl]-5-(phenylsulfonyl)benzenesulfonamide;
N-[1-(3-Fluorobenzoyl)piperidin-4-yl]-2-isopropyl-5-(phenylsulfonyl)benzenesulfonamide;
N-[1-(4-Fluorobenzoyl)piperidin-4-yl]-2-isopropyl-5-(phenylsulfonyl)benzenesulfonamide;
N-[1-(2-Fluorobenzoyl)piperidin-4-yl]-2-isopropyl-5-(phenylsulfonyl)benzenesulfonamide; 2-Isopropyl-5-(phenylsulfonyl)-N-{1-[4-(trifluoromethyl)benzoyl]piperidin-4-yl}benzene sulfonamide;
2-Isopropyl-N-[1-(1-naphthoyl)piperidin-4-yl]-5-(phenylsulfonyl)benzenesulfonamide;
2-Isopropyl-N-[1-(2-naphthoyl)piperidin-4-yl]-5-(phenylsulfonyl)benzenesulfonamide;
N-[1-(3-Cyanobenzoyl)piperidin-4-yl]-2-isopropyl-5-(phenylsulfonyl)benzenesulfonamide;
N-[1-(4-Cyanobenzoyl)piperidin-4-yl-2-isopropyl-5-(phenylsulfonyl)benzenesulfonamide;
N-[1-(4-tert-Butylbenzoyl)piperidin-4-yl]-2-isopropyl-5-(phenylsulfonyl)benzenesulfonamide;
N-[1-(2-Ethoxy-1-naphthoyl)piperidin-4-yl]-2-isopropyl-5-(phenylsulfonyl)benzenesulfonamide;
N-[1-(2-chloro-6-methylisonicotinoyl)piperidin-4-yl]-5-(phenylsulfonyl)-2-(trifluoromethyl)benzenesulfonamide;
N-[1-(2,6-dichloroisonicotinoyl)piperidin-4-yl]-5-(phenylsulfonyl)-2-(trifluoromethyl)benzenesulfonamide;
N-{1-[4-(dimethylamino)benzoyl]piperidin-4-yl}-5-(phenylsulfonyl)-2-(trifluoromethyl)benzenesulfonamide;
N-{1-[(6-chloropyridin-3-yl)carbonyl]piperidin-4-yl]-5-(phenylsulfonyl)-2-(trifluoromethyl)benzenesulfonamide;
N-{1-[(2,5-dichloropyridin-3-yl)carbonyl]piperidin-4-yl}-5-(phenylsulfonyl)-2-(trifluoromethyl)benzenesulfonamide;
N-[1-(2-chloroisonicotinoyl)piperidin-4-yl]-5-(phenylsulfonyl)-2-(trifluoromethyl)benzenesulfonamide;
5-(phenylsulfonyl)-N-{1-[(6-pyrrolidin-1-ylpyridin-3-yl)carbonyl]piperidin-4-yl}-2-(trifluoromethyl)benzenesulfonamide;
N-(1-{6-(dimethylamino)pyridin-3-yl]carbonyl}piperidin-4-yl)-5-(phenylsulfonyl)-2-(trifluoromethyl)benzenesulfonamide;
N-{1-[(6-oxo-1-{5-(phenylsulfonyl)-2-(trifluoromethyl)phenyl]sulfonyl}-1,6-dihydropyridin-3-yl)carbonyl]piperidin-4-yl}-5-(phenylsulfonyl)-2-(trifluoromethyl)benzenesulfonamide;
N-{1-[(6-phenylpyridin-3-yl)carbonyl]piperidin-4-yl}-5-(phenylsulfonyl)-2-(trifluoromethyl)benzenesulfonamide;
N-{1-[(6-morpholin-4-ylpyridin-3-yl)carbonyl]piperidin-4-yl}-5-(phenylsulfonyl)-2-(trifluoromethyl)benzenesulfonamide;
5-(phenylsulfonyl)-N-[1-(2-pyrrolidin-1-ylisonicotinoyl)piperidin-4-yl]-2-(trifluoromethyl)benzenesulfonamide;
N-{1-[(6-oxo-1,6-dihydropyridin-3-yl)carbonyl]piperidin-4-yl}-5-(phenylsulfonyl)-2-(trifluoromethyl)benzenesulfonamide;
5-(phenylsulfonyl)-N-[1-(pyridin-3-ylcarbonyl)piperidin-4-yl]-2-(trifluoromethyl)benzenesulfonamide;
5-(phenylsulfonyl)-N-[1-(pyridin-2-ylcarbonyl)piperidin-4-yl]-2-(trifluoromethyl)benzenesulfonamide;
N-{1-[4-(methylthio)benzoyl]piperidin-4-yl}-5-(phenylsulfonyl)-2-(trifluoromethyl)benzenesulfonamide;
5-(phenylsulfonyl)-2-(trifluoromethyl)-N-(1-{[6-(trifluoromethyl)pyridin-3-yl]carbonyl}piperidin-4-yl)benzenesulfonamide;
N-{1-[4-(methylsulfinyl)benzoyl]piperidin-4-yl}-5-(phenylsulfonyl)-2-(trifluoromethyl)benzenesulfonamide;
5-(phenylsulfonyl)-N-[1-(1,3-thiazol4-ylcarbonyl)piperidin-4-yl]-2-(trifluoromethyl)benzenesulfonamide;
tert-butyl 4-({[5-(phenylsulfonyl)-2-(trifluoromethyl)phenyl]sulfonyl}amino)piperidine-1-carboxylate;
tert-butyl (3S)-3-({[5-(phenylsulfonyl)-2-(trifluoromethyl)phenyl]sulfonyl}amino)piperidine-1-carboxylate;
tert-butyl (3R)-3-({[5-(phenylsulfonyl)-2-(trifluoromethyl)phenyl]sulfonyl}amino)piperidine-1-carboxylate;
tert-butyl 4-[({5-[(4-fluorophenyl)sulfonyl]-2-methylphenyl}sulfonyl)amino]piperidine-1-carboxylate;
tert-butyl (3S)-3-({[5-(phenylsulfonyl)-2-(trifluoromethyl)phenyl]sulfonyl}amino)pyrrolidine-1-carboxylate;
tert-butyl (3R)-3-({[5-(phenylsulfonyl)-2-(trifluoromethyl)phenyl]sulfonyl}amino)pyrrolidine-1-carboxylate;
tert-butyl 4-({[5-[(4-fluorophenyl)sulfonyl]-2-(trifluoromethyl)phenyl]sulfonyl}amino)piperidine-1-carboxylate;
tert-butyl 4-({[2-ethyl-5-(phenylsulfonyl)phenyl]sulfonyl}amino)piperidine-1-carboxylate;
tert-butyl 4-({[2,3-dimethyl-5-(phenylsulfonyl)phenyl]sulfonyl}amino)piperidine-1-carboxylate;
tert-butyl 4-({[2,4-dimethyl-5-(phenylsulfonyl)phenyl]sulfonyl}amino)piperidine-1-carboxylate;
tert-butyl 4-({[2-isopropyl-5-(phenylsulfonyl)phenyl]sulfonyl}amino)piperidine-1-carboxylate;
4-{3-({[1-(tert-butoxycarbonyl)piperidin-4-yl]amino }sulfonyl)-4-(trifluoromethyl)phenyl]sulfonyl}benzoic acid;
4-oxo-4-[4-({[5-(phenylsulfonyl)-2-(trifluoromethyl)phenyl]sulfonyl}amino)piperidin-1-yl]butanoic acid;
4-oxo4-[3-({[5-(phenylsulfonyl)-2-(trifluoromethyl)phenyl]sulfonyl}amino)pyrrolidin-1-yl]butanoic acid;
5-oxo-5-[4-({[5-(phenylsulfonyl)-2-(trifluoromethyl)phenyl]sulfonyl}amino)piperidin-1-yl]pentanoic acid;
5-oxo-5-[3-({[5-(phenylsulfonyl)-2-(trifluoromethyl)phenyl]sulfonyl}amino)pyrrolidin-1-yl]pentanoic acid;
N-[1-(N,N-dimethylglycyl)piperidin-4-yl]-5-(phenylsulfonyl)-2-(trifluoromethyl)benzenesulfonamide;
tert-butyl 4-{2-oxo-2-[4-({[5-(phenylsulfonyl)-2-(trifluoromethyl)phenyl]sulfonyl}amino)piperidin-1-yl]ethyl}piperidine-1-carboxylate;
5-(phenylsulfonyl)-N-[1-(piperidin-4-ylacetyl)piperidin-4-yl]-2-(trifluoromethyl)benzenesulfonamide;
N-[1-(N-methylglycyl)piperidin-4-yl]-5-(phenylsulfonyl)-2-(trifluoromethyl)benzenesulfonamide;
5-(phenylsulfonyl)-N-[1-(pyrrolidin-1-ylacetyl)piperidin-4-yl]-2-(trifluoromethyl)benzenesulfonamide;
N-[1-(morpholin-4-ylacetyl)piperidin-4-yl]-5-(phenylsulfonyl)-2-(trifluoromethyl)benzenesulfonamide;
5-(phenylsulfonyl)-N-[1-(piperazin-1-ylacetyl)piperidin-4-yl]-2-(trifluoromethyl)benzenesulfonamide;
N-{1-[3-(methylthio)propanoyl]piperidin-4-yl}-5-(phenylsulfonyl)-2-(trifluoromethyl)benzenesulfonamide;
N-{1-[3-(methylsulfinyl)propanoyl]piperidin-4-yl}-5-(phenylsulfonyl)-2-(trifluoromethyl)benzenesulfonamide;
N-[1-(1H-imidazol-1-ylacetyl)piperidin-4-yl]-5-(phenylsulfonyl)-2-(trifluoromethyl)benzenesulfonamide;
4-[({5-[(4-fluorophenyl)sulfonyl]-2-isopropylphenyl}sulfonyl)amino]-N-1-naphthylpiperidine-1-carbothioamide;
N-(2-fluorophenyl)-4-[({5-[(4-fluorophenyl)sulfonyl]-2-isopropylphenyl}sulfonyl)amino]piperidine-1-carbothioamide;
4-[({5-[(4-fluorophenyl)sulfonyl]-2-isopropylphenyl}sulfonyl)amino]-N-(2-methylphenyl)piperidine-1-carbothioamide;
ethyl ({4-[({5-[(4-fluorophenyl)sulfonyl]-2-isopropylphenyl}sulfonyl)amino]piperidin-1-yl}carbonothioyl)carbamate;
N-butyl-4-[({5-[(4-fluorophenyl)sulfonyl]-2-isopropylphenyl}sulfonyl)amino]piperidine-1-carbothioamide;
4-[({5-[(4-fluorophenyl)sulfonyl]-2-isopropylphenyl}sulfonyl)amino]-N-(4-methoxyphenyl)piperidine-1-carbothioamide;
methyl 4-[({4-[({5-[(4-fluorophenyl)sulfonyl]-2-isopropylphenyl}sulfonyl)amino]piperidin-1-yl}carbonothioyl)amino]benzoate;
methyl N-({4-[({5-[(4-fluorophenyl)sulfonyl]-2-isopropylphenyl}sulfonyl)amino]piperidin-yl}carbonothioyl)glycinate;
4-[({5-[(4-fluorophenyl)sulfonyl]-2-isopropylphenyl}sulfonyl)amino]-N-(2-morpholin-4-ylethyl)piperidine-1-carbothioamide;
4-[({5-[(4-fluorophenyl)sulfonyl]-2-isopropylphenyl}sulfonyl)amino]-N-(3-nitrophenyl)piperidine-1-carbothioamide;
3-[({4-[({5-[(4-fluorophenyl)sulfonyl]-2-isopropylphenyl}sulfonyl)amino]piperidin-1-yl}carbonothioyl)amino]benzoic acid;
4-[({5-[(4-fluorophenyl)sulfonyl]-2-isopropylphenyl}sulfonyl)amino]-N-pyridin-3-ylpiperidine-1-carbothioamide;
4-[({5-[(4-fluorophenyl)sulfonyl]-2-isopropylphenyl}sulfonyl)amino]-N-[4-(trifluoromethyl)phenyl]piperidine-1-carbothioamide;
N-(4-tert-butylphenyl)-4-({[5-(phenylsulfonyl)-2-(trifluoromethyl)phenyl]sulfonyl}amino)piperidine-1-carboxamide;
3-({[4-({[5-(phenylsulfonyl)-2-(trifluoromethyl)phenyl]sulfonyl}amino)piperidin-1-yl]carbonothioyl}amino)benzoic acid;
tert-butyl 4-{4-({[5-(phenylsulfonyl)-2-(trifluoromethyl)phenyl]sulfonyl}amino)piperidin-1-yl]carbonyl}piperidine-1-carboxylate;
5-(phenylsulfonyl)-N-[1-(piperidin-4-ylcarbonyl)piperidin-4-yl]-2-(trifluoromethyl)benzenesulfonamide;
tert-butyl (2S)-2-{[4-({[5-(phenylsulfonyl)-2-(trifluoromethyl)phenyl]sulfonyl}amino)piperidin-1-yl]carbonyl}pyrrolidine-1-carboxylate;
5-(phenylsulfonyl)-N-(1-L-prolylpiperidin-4-yl)-2-(trifluoromethyl)benzenesulfonamide;
tert-butyl (2R)-2-{4-({[5-(phenylsulfonyl)-2-(trifluoromethyl)phenyl]sulfonyl}amino)piperidin-1-yl]carbonyl}pyrrolidine-1-carboxylate;
5-(phenylsulfonyl)-N-(1-D-prolylpiperidin-4-yl)-2-(trifluoromethyl)benzenesulfonamide;
N-[1-(1-acetyl-L-prolyl)piperidin-4-yl]-5-(phenylsulfonyl)-2-(trifluoromethyl)benzenesulfonamide;
tert-butyl (5S)-2-oxo-5-{[4-({[5-(phenylsulfonyl)-2-(trifluoromethyl)phenyl]sulfonyl}amino)piperidin-1-yl]carbonyl}pyrrolidine-1-carboxylate;
N-[1-(5-oxo-L-prolyl)piperidin-4-yl]-5-(phenylsulfonyl)-2-(trifluoromethyl)benzenesulfonamide;
N-[1-(1-methyl-L-prolyl)piperidin-4-yl]-5-(phenylsulfonyl)-2-(trifluoromethyl)benzenesulfonamide;
tert-butyl (4R)-4-{4-({[5-(phenylsulfonyl)-2-(trifluoromethyl)phenyl]sulfonyl}amino)piperidin-1-yl]carbonyl}-1,3-thiazolidine-3-carboxylate;
5-(phenylsulfonyl)-N-{1-[(4R)-1,3-thiazolidin-4-ylcarbonyl]piperidin-4-yl}-2-(trifluoromethyl)benzenesulfonamide;
tert-butyl (3R)-3-{4-({[5-(phenylsulfonyl)-2-(trifluoromethyl)phenyl]sulfonyl}amino)piperidin-1-yl]carbonyl}pyrrolidine-1-carboxylate;
tert-butyl (3S)-3-{4-({[5-(phenylsulfonyl)-2-(trifluoromethyl)phenyl]sulfonyl}amino)piperidin-1-yl]carbonyl}pyrrolidine-1-carboxylate;
5-(phenylsulfonyl)-N-{1-[(3R)-pyrrolidin-3-ylcarbonyl]piperidin-4-yl}-2-(trifluoromethyl)benzenesulfonamide;
5-(phenylsulfonyl)-N-{1-[(3S)-pyrrolidin-3-ylcarbonyl]piperidin-4-yl}-2-(trifluoromethyl)benzenesulfonamide;
tert-butyl (4R)-4-{4-({[5-(phenylsulfonyl)-2-(trifluoromethyl)phenyl]sulfonyl}amino)piperidin-1-yl]carbonyl}-1,3-thiazolidine-3-carboxylate 1-oxide;
N-(1-{[(3R)-1-acetylpyrrolidin-3-yl]carbonyl piperidin-4-yl)-5-(phenylsulfonyl)-2-(trifluoromethyl)benzenesulfonamide;
N-(1-{[(3S)-1-acetylpyrrolidin-3-yl]carbonyl}piperidin-4-yl)-5-(phenylsulfonyl)-2-(trifluoromethyl)benzenesulfonamide;
N-[1-(1-isobutyryl-L-prolyl)piperidin-4-yl]-5-(phenylsulfonyl)-2-(trifluoromethyl)benzenesulfonamide;
N-{1-[1-(2,2-dimethylpropanoyl)-L-prolyl]piperidin-4-yl}-5-(phenylsulfonyl)-2-(trifluoromethyl)benzenesulfonamide;
N-{1-[1-(3,3-dimethylbutanoyl)-L-prolyl]piperidin-4-yl}-5-(phenylsulfonyl)-2-(trifluoromethyl)benzenesulfonamide;
N-1-[1-(cyclohexylcarbonyl)-L-prolyl]piperidin-4-yl}-5-(phenylsulfonyl)-2-(trifluoromethyl)benzenesulfonamide;
N-{1-[1-(morpholin-4-ylcarbonyl)-L-prolyl]piperidin-4-yl}-5-(phenylsulfonyl)-2-(trifluoromethyl)benzenesulfonamide;
(2S)-N-(tert-butyl)-2-{[4-({[5-(phenylsulfonyl)-2-(trifluoromethyl)phenyl]sulfonyl}amino)piperidin-1-yl]carbonyl}pyrrolidine-1-carboxamide;
(2S)-N-phenyl-2-{4-({[5-(phenylsulfonyl)-2-(trifluoromethyl)phenyl]sulfonyl}amino)piperidin-1-yl]carbonyl}pyrrolidine-1-carboxamide;
N-{1-[1-(methylsulfonyl)-L-prolyl]piperidin-4-yl}-5-(phenylsulfonyl)-2-(trifluoromethyl)benzenesulfonamide;
N-[1-(1-benzoyl-L-prolyl)piperidin-4-yl]-5-(phenylsulfonyl)-2-(trifluoromethyl)benzenesulfonamide;
N-(1-{1-[4-(dimethylamino)benzoyl]-L-prolyl}piperidin-4-yl)-5-(phenylsulfonyl)-2-(trifluoromethyl)benzenesulfonamide;
N-[1-(1-isonicotinoyl-L-prolyl)piperidin-4-yl]-5-(phenylsulfonyl)-2-(trifluoromethyl)benzenesulfonamide;
N-(1-{1-[(6-chloropyridin-3-yl)carbonyl]-L-prolyl}piperidin-4-yl)-5-(phenylsulfonyl)-2-(trifluoromethyl)benzenesulfonamide;
4-[((2S)-2-{4-({[5-(phenylsulfonyl)-2-(trifluoromethyl)phenyl]sulfonyl}amino)piperidin-1-yl]carbonyl}pyrrolidin-1-yl)sulfonyl]benzoic acid;
N-[1-(N,N-dimethylglycyl-L-prolyl)piperidin-4-yl]-5-(phenylsulfonyl)-2-(trifluoromethyl)benzenesulfonamide;
N-[1-(1-benzyl-L-prolyl)piperidin-4-yl]-5-(phenylsulfonyl)-2-(trifluoromethyl)benzenesulfonamide;
N-{1-[1-(cyclohexylmethyl)-L-prolyl]piperidin-4-yl}-5-(phenylsulfonyl)-2-(trifluoromethyl)benzenesulfonamide;
N-{1-[1-(3,3-dimethylbutyl)-L-prolyl]piperidin-4-yl}-5-(phenylsulfonyl)-2-(trifluoromethyl)benzenesulfonamide;
(2S)-N-ethyl-2-{4-({[5-(phenylsulfonyl)-2-(trifluoromethyl)phenyl]sulfonyl}amino)piperidin-1-yl]carbonyl}pyrrolidine-1-carboxamide;
(2S)-N,N-dimethyl-2-{[4-({[5-(phenylsulfonyl)-2-(trifluoromethyl)phenyl]sulfonyl}amino)piperidin-1-yl]carbonyl}pyrrolidine-1-carboxamide;
tert-butyl (2S)-2-{[4-({[5-[(3-cyanophenyl)sulfonyl]-2-(trifluoromethyl)phenyl]sulfonyl}amino)piperidin-1-yl]carbonyl}pyrrolidine-1-carboxylate;
N-[1-(2-hydroxy-2-methylpropyl)piperidin-4-yl]-5-(phenylsulfonyl)-2-(trifluoromethyl)benzenesulfonamide;
5-(phenylsulfonyl)-2-(trifluoromethyl)-N-{1-[4-(trifluoromethyl)benzyl]piperidin-4-yl}benzenesulfonamide;
N-[1-(cyanomethyl)piperidin-4-yl]-5-(phenylsulfonyl)-2-(tuoromethyl)benzenesulfonamide;
N-[1-(2-oxo-2-phenylethyl)piperidin-4-yl]-5-(phenylsulfonyl)-2-(trifluoromethyl)benzenesulfonamide;
5-(phenylsulfonyl)-2-(trifluoromethyl)-N-{1-[4-(trifluoromethyl)phenyl]piperidin-4-yl}benzenesulfonamide;
N-[1-(2-hydroxyethyl)piperidin-4-yl]-5-(phenylsulfonyl)-2-(trifluoromethyl)benzenesulfonamide;
2-Isopropyl-N-[(1R*,5S*)-8-methyl-8-azabicyclo[3.2.1]oct-3-yl]-5-(phenylsulfonyl)benzenesulfonamide monohydrochloride;
[4-({[5-(phenylsulfonyl)-2-(trifluoromethyl)phenyl]sulfonyl}amino)piperidin-1-yl]acetic acid;
2-[4-({[5-(phenylsulfonyl)-2-(trifluoromethyl)phenyl]sulfonyl}amino)piperidin-1-yl]acetamide;
5-(phenylsulfonyl)-N-[1-(2H-tetrazol-5-ylmethyl)piperidin-4-yl]-2-(trifluoromethyl)benzenesulfonamide;
3-[4-({[5-(phenylsulfonyl)-2-(trifluoromethyl)phenyl]sulfonyl}amino)piperidin-1-yl]propanoic acid;
3-[4-({[5-(phenylsulfonyl)-2-(trifluoromethyl)phenyl]sulfonyl}amino)piperidin-1-yl]benzoic acid;
4-[4-({[5-(phenylsulfonyl)-2-(trifluoromethyl)phenyl]sulfonyl}amino)piperidin-1-yl]benzoic acid;
N-[1-(3-cyanophenyl)piperidin-4-yl -5-(phenylsulfonyl)-2-(trifluoromethyl)benzenesulfonamide;
2-[4-({[5-(phenylsulfonyl)-2-(trifluoromethyl)phenyl]sulfonyl}amino)piperidin-1-yl]propanamide;
N-[1-(2-morpholin-4-ylethyl)piperidin-4-yl]-5-(phenylsulfonyl)-2-(trifluoromethyl)benzenesulfonamide;
N-[(1R *, 5S *)-8-Methyl-8-azabicyclo[3.2.1]oct-3-yl]-5-(phenylsulfonyl)-2-(trifluoromethyl)benzenesulfonamide;
(2R)-2-[4-({[5-(phenylsulfonyl)-2-(trifluoromethyl)phenyl]sulfonyl}amino)piperidin-1-yl]propanamide;
(2S)-2-(4-({[5-(phenylsulfonyl)-2-(trifluoromethyl)phenyl]sulfonyl}amino)piperidin-1-yl]propanamide;
methyl [4-({[5-(phenylsulfonyl)-2-(trifluoromethyl)phenyl]sulfonyl}amino)piperidin-1-yl]acetate;
N-methyl-2-[4-({[5-(phenylsulfonyl)-2-(trifluoromethyl)phenyl]sulfonyl}amino)piperidin-1-yl]acetamide;
N,N-dimethyl-2-[4-({[5-(phenylsulfonyl)-2-(trifluoromethyl)phenyl]sulfonyl}amino)piperidin-1-yl]acetamide;
N-isopropyl-2-[4-({[5-(phenylsulfonyl)-2-(trifluoromethyl)phenyl]sulfonyl}amino)piperidin-1-yl]acetamide;
N-[1-(2-morpholin-4-yl-2-oxoethyl)piperidin-4-yl]-5-(phenylsulfonyl)-2-(trifluoromethyl)benzenesulfonamide;
N-{1-[(4-tert-butylphenyl)sulfonyl]piperidin-4-yl}-5-(phenylsulfonyl)-2-(trifluoromethyl)benzenesulfonamide;
5-(phenylsulfonyl)-N-[1-(phenylsulfonyl)piperidin-4-yl]-2-(trifluoromethyl)benzenesulfonamide;
3-{[4-({[5-(phenylsulfonyl)-2-(trifluoromethyl)phenyl]sulfonyl}amino)piperidin-1-yl]sulfonyl}benzoic acid;
4-{4-({[5-(phenylsulfonyl)-2-(trifluoromethyl)phenyl]sulfonyl}amino)piperidin-1-yl]sulfonyl}benzoic acid;
N-{1-[(4-hydroxyphenyl)sulfonyl]piperidin-4-yl}-5-(phenylsulfonyl)-2-(trifluoromethyl)benzenesulfonamide;
methyl 3-{4-({[5-(phenylsulfonyl)-2-(trifluoromethyl)phenyl]sulfonyl}amino)piperidin-1-yl]sulfonyl}benzoate;
N-{1-[(3-cyanophenyl)sulfonyl]piperidin-4-yl}-5-(phenylsulfonyl)-2-(trifluoromethyl)benzenesulfonamide;
5-(phenylsulfonyl)-N-(1-{3-(2H-tetrazol-5-yl)phenyl]sulfonyl}piperidin-4-yl)-2-(trifluoromethyl)benzenesulfonamide;
5-(phenylsulfonyl)-N-(2-pyridin-3-ylethyl)-2-(trifluoromethoxy)benzenesulfonamide;
2-isopropyl-5-(phenylsulfonyl)-N-(2-pyridin-2-ylethyl)benzenesulfonamide;
2-isopropyl-5-(phenylsulfonyl)-N-(2-pyridin-3-ylethyl)benzenesulfonamide;
5-(phenylsulfonyl)-N-(2-pyridin-3-ylethyl)-2-(trifluoromethyl)benzenesulfonamide;
1-Methoxy-4-(phenylsulfonyl)benzene;
N-[2-(1-oxidopyridin-3-yl)ethyl]-5-(phenylsulfonyl)-2-(trifluoromethyl)benzenesulfonamide;
5-[(4-fluorophenyl)sulfonyl]-N-(2-hydroxy-2-pyridin-2-ylethyl)-2-isopropylbenzenesulfonamide;
N-(2-hydroxy-2-pyridin-2-ylethyl)-2-isopropyl-5-(phenylsulfonyl)benzene-sulfonamide;
N-(2-hydroxy-2-pyridin-2-ylethyl)-2,4-dimethyl-5-(phenylsulfonyl)benzenesulfonamide;
5-[(4-fluorophenyl)sulfonyl]-N-(2-hydroxy-2-pyridin-3-ylethyl)-2-isopropylbenzenesulfonamide;
N-(2-hydroxy-2-pyridin-3-ylethyl)-2-isopropyl-5-(phenylsulfonyl)benzenesulfonamide;
N-(2-hydroxy-2-pyridin-3-ylethyl)-2,4-dimethyl-5-(phenylsulfonyl)benzenesulfonamide;
N-[2-(1H-Imidazol-1-yl)ethyl]-5-(phenylsulfonyl)-2-(trifluoromethyl)benzenesulfonamide;
5-[(4-fluorophenyl)sulfonyl]-N-(2-hydroxy-2-pyridin-3-ylethyl)-2-methylbenzenesulfonamide;
N-(2-hydroxy-2-pyridin-3-ylethyl)-5-(phenylsulfonyl)-2-(trifluoromethyl)benzenesulfonamide;
5-[(3-methoxyphenyl)sulfonyl]-N-(2-pyridin-3-ylethyl)-2-(trifluoromethyl)benzenesulfonamide;
5-[(3-chlorophenyl)sulfonyl]-N-(2-pyridin-3-ylethyl)-2-(trifluoromethyl)benzenesulfonamide;
5-[(4-fluorophenyl)sulfonyl]-N-(2-hydroxy-2-pyridin-2-ylethyl)-2-methylbenzenesulfonamide;
N-(2-hydroxy-2-pyridin-2-ylethyl)-5-(phenylsulfonyl)-2-(trifluoromethyl)benzenesulfonamide;
5-(phenylsulfonyl)-N-(2-pyridin-4-ylethyl)-2-(trifluoromethyl)benzenesulfonamide;
5-[(4-hydroxyphenyl)sulfonyl]-N-(2-pyridin-3-ylethyl)-2-(trifluoromethyl)benzenesulfonamide;
5-[(4-fluorophenyl)sulfonyl]-N-[2-(1H-imidazol-1-yl)ethyl]-2-(trifluoromethyl)benzenesulfonamide;
5-[(2-fluorophenyl)sulfonyl]-N-[2-(1H-imidazol-1-yl)ethyl]-2-(trifluoromethyl)benzenesulfonamide;
5-(Phenylsulfonyl)-2-propyl-N-(2-pyridin-4-ylethyl)benzenesulfonamide;
5-(phenylsulfonyl)-N-[2-(2H-tetrazol-5-yl)ethyl]72-(trifluoromethyl)benzenesulfonamide;
5-[(4-Fluorophenyl)sulfonyl]-2-propyl-N-(2-pyridin -3-ylethyl)benzenesulfonamide;
5-[(4-Fluorophenyl)sulfonyl]-2-propyl-N-(2-pyridin-4-ylethyl)benzenesulfonamide;
5-(Phenylsulfonyl)-2-propyl-N-(2-pyridin-3-ylethyl)benzenesulfonamide;
2-isopropyl-5-(phenylsulfonyl)-N-(pyridin-4-ylmethyl)benzenesulfonamide;
5-(phenylsulfonyl)-N-(pyridin-4-ylmethyl)-2-(trifluoromethyl)benzenesulfonamide;
N-[(6-chloropyridin-3-yl)methyl]-5-(phenylsulfonyl)-2-(trifluoromethyl)benzenesulfonamide;
5-(phenylsulfonyl)-N-[(6-pyrrolidin-1-ylpyridin-3-yl)methyl]-2-(trifluoromethyl)benzenesulfonamide;
N-[(6-morpholin-4-ylpyridin-3-yl)methyl]-5-(phenylsulfonyl)-2-(trifluoromethyl)benzenesulfonamide;
2-chloro-N-[(1R*,2R*)-2-hydroxy-1-methyl-2-phenylethyl]-5-(phenylsulfonyl)benzenesulfonamide;
N-(2-hydroxy-2-phenylethyl)-5-(phenylsulfonyl)-2-(trifluoromethyl)benzenesulfonamide;
N-[(1R*,2R*)-2-hydroxy-1-methyl-2-phenylethyl]-5-(phenylsulfonyl)-2-(trifluoromethyl)benzenesulfonamide;
N-[(1S,2R)-2-hydroxy-1-methyl-2-phenylethyl]-5-(phenylsulfonyl)-2-(trifluoromethyl)benzenesulfonamide;
N-[(1R,2S)-2-hydroxy-1-methyl-2-phenylethyl]-5-(phenylsulfonyl)-2-(trifluoromethyl)benzenesulfonamide;
N-[(1S)-1-benzyl-2-hydroxyethyl]-5-(phenylsulfonyl)-2-(trifluoromethyl)benzenesulfonamide;
N-[(1S)-2-hydroxy-1-(1H-indol-3-ylmethyl)ethyl]-5-(phenylsulfonyl)-2-(trifluoromethyl)benzenesulfonamide;
5-[(4-fluorophenyl)sulfonyl]-N-(2-hydroxy-2-phenylethyl)-2-isopropylbenzenesulfonamide;
N-(2-hydroxy-2-phenylethyl)-2-isopropyl-5-(phenylsulfonyl)benzenesulfonamide;
N-[(2R)-2-hydroxy-2-phenylethyl]-5-(phenylsulfonyl)-2-(trifluoromethyl)benzenesulfonamide;
N-[(2S)-2-hydroxy-2-phenylethyl]-5-(phenylsulfonyl)-2-(trifluoromethyl)benzenesulfonamide;
4-[2-({[5-(phenylsulfonyl)-2-(trifluoromethyl)phenyl]sulfonyl}amino)ethyl]benzoic acid;
N-[2-(4-aminophenyl)ethyl]-5-(phenylsulfonyl)-2-(trifluoromethyl)benzenesulfonamide;
N-[2-(4-methoxyphenyl)ethyl]-5-(phenylsulfonyl)-2-(trifluoromethyl)benzenesulfonamide;
N-(4-aminobenzyl)-5-(phenylsulfonyl)-2-(trifluoromethyl)benzenesulfonamide;
N-[2-(4-hydroxyphenyl)ethyl]-5-(phenylsulfonyl)-2-(trifluoromethyl)benzenesulfonamide;
methyl 4-[({[5-(phenylsulfonyl)-2-(trifluoromethyl)phenyl]sulfonyl}amino)methyl]benzoate;
methyl 4-[({[2-isopropyl-5-(phenylsulfonyl)phenyl]sulfonyl}amino)methyl]benzoate;
N-[2-(4-bromophenyl)ethyl]-5-(phenylsulfonyl)-2-(trifluoromethyl)benzenesulfonamide;
3-[({4-[({[5-(phenylsulfonyl)-2-(trifluoromethyl)phenyl]sulfonyl}amino)methyl]phenyl}amino)sulfonyl]benzoic acid;
N-(4-bromobenzyl)-5-(phenylsulfonyl)-2-(trifluoromethyl)benzenesulfonamide;
N-[2-(4-cyanophenyl)ethyl]-5-(phenylsulfonyl)-2-(trifluoromethyl)benzenesulfonamide;
methyl 4-[2-({[5-(phenylsulfonyl)-2-(trifluoromethyl)phenyl]sulfonyl}amino)ethyl]benzoate;
4′-[({[5-(phenylsulfonyl)-2-(trifluoromethyl)phenyl]sulfonyl}amino)methyl]biphenyl-3-carboxylic acid;
4′-[({[5-(phenylsulfonyl)-2-(trifluoromethyl)phenyl]sulfonyl}amino)methyl]biphenyl-4-carboxylic acid;
5-(phenylsulfonyl)-N-{2-[4-(2H-tetrazol-5-yl)phenyl]ethyl}-2-(trifluoromethyl)benzenesulfonamide;
N-(2-{4-[(methylsulfonyl)amino]phenyl}ethyl)-5-(phenylsulfonyl)-2-(trifluoromethyl)benzenesulfonamide;
N-(3-bromobenzyl)-5-(phenylsulfonyl)-2-(trifluoromethyl)benzenesulfonamide;
3′-[({[5-(phenylsulfonyl)-2-(trifluoromethyl)phenyl]sulfonyl}amino)methyl]biphenyl-3-carboxylic acid;
3′-[({[5-(phenylsulfonyl)-2-(trifluoromethyl)phenyl]sulfonyl}amino)methyl]biphenyl-4-carboxylic acid;
N-[2-(4-{[amino(imino)methyl]amino}phenyl)ethyl]-5-(phenylsulfonyl)-2-(trifluoromethyl)benzenesulfonamide;
N-[4-(dimethylamino)benzyl]-5-(phenylsulfonyl)-2-(trifluoromethyl)benzenesulfonamide;
N-(2,4-dimethoxybenzyl)-5-(phenylsulfonyl)-2-(trifluoromethyl)benzenesulfonamide;
tert-Butyl [2-({[5-(phenylsulfonyl)-2-(trifluoromethyl)phenyl]sulfonyl}amino)ethyl]carbamate;
N-(2-Aminoethyl)-5-(phenylsulfonyl)-2-(trifluoromethyl)benzenesulfonamide hydrochloride;
N-[2-({[5-(Phenylsulfonyl)-2-(trifluoromethyl)phenyl]sulfonyl}amino)ethyl]benzamide;
4-Methyl-N-[2-({[5-(phenylsulfonyl)-2-(trifluoromethyl)phenyl]sulfonyl}amino)ethyl]benzamide;
4-tert-Butyl-N-[2-({[5-(phenylsulfonyl)-2-(trifluoromethyl)phenyl]sulfonyl}amino)ethyl]benzamide;
4-Fluoro-N-[2-({[5-(phenylsulfonyl)-2-(trifluoromethyl)phenyl]sulfonyl}amino)ethyl]benzamide;
4-Chloro-N-[2-({[5-(phenylsulfonyl)-2-(trifluoromethyl)phenyl]sulfonyl}amino)ethyl]benzamide;
4-Bromo-N-[2-({[5-(phenylsulfonyl)-2-(trifluoromethyl)phenyl]sulfonyl}amino)ethyl]benzamide;
4-Methoxy-N-[2-({[5-(phenylsulfonyl)-2-(trifluoromethyl)phenyl]sulfonyl}amino)ethyl]benzamide;
N-[2-({[5-(Phenylsulfonyl)-2-(trifluoromethyl)phenyl]sulfonyl}amino)ethyl]-4-(trifluoromethyl)benzamide;
N-[2-({[5-(Phenylsulfonyl)-2-(trifluoromethyl)phenyl]sulfonyl}amino)ethyl]-4-(trifluoromethoxy)benzamide;
N-[2-({[5-(Phenylsulfonyl)-2-(trifluoromethyl)phenyl]sulfonyl}amino)ethyl]isonicotinamide;
tert-Butyl methyl[2-({[5-(phenylsulfonyl)-2-(trifluoromethyl)phenyl]sulfonyl}amino)ethyl]carbamate;
N-[2-(Methylamino)ethyl]-5-(phenylsulfonyl)-2-(trifluoromethyl)benzenesulfonamide hydrochloride;
N-Methyl-N-[2-({[5-(phenylsulfonyl)-2-(trifluoromethyl)phenyl]sulfonyl}amino)ethyl]benzamide;
4-Methoxy-N-methyl-N-[2-({[5-(phenylsulfonyl)-2-(trifluoromethyl)phenyl]sulfonyl}amino)ethyl]benzamide;
N-{2-[(anilinocarbonyl)(methyl)amino]ethyl}-5-(phenylsulfonyl)-2-(trifluoromethyl)benzenesulfonamide;
N-(2-{methyl[(pyridin-3-ylamino)carbonyl]amino}ethyl)-5-(phenylsulfonyl)-2-(trifluoromethyl)benzenesulfonamide;
N-{2-[{[(2,4-dimethoxyphenyl)amino]carbonyl}(methyl)amino]ethyl}-5-(phenylsulfonyl)-2-(trifluoromethyl)benzenesulfonamide;
N-{2-[[(tert-butylamino)carbonyl](methyl)amino]ethyl}-5-(phenylsulfonyl)-2-(trifluoromethyl)benzenesulfonamide;
N-{2-[{[(4-methoxyphenyl)amino]carbonyl}(methyl)amino]ethyl}-5-(phenylsulfonyl)-2-(trifluoromethyl)benzenesulfonamide;;
N-{2-[[(butylamino)carbonyl](methyl)amino]ethyl}-5-(phenylsulfonyl)-2-(trifluoromethyl)benzenesulfonamide N-{2-[{[(2,4-difluorophenyl)amino]carbonyl}(methyl)amino]ethyl}-5-(phenylsulfonyl)-2-(trifluoromethyl)benzenesulfonamide;
N-methyl-N-[2-({[5-(phenylsulfonyl)-2-(trifluoromethyl)phenyl]sulfonyl}amino)ethyl]pyrrolidine-1-carboxamide;
N-{2-[[(diethylamino)carbonyl](methyl)amino]ethyl}-5-(phenylsulfonyl)-2-(trifluoromethyl)benzenesulfonamide;
N-methyl-N-[2-({[5-(phenylsulfonyl)-2-(trifluoromethyl)phenyl]sulfonyl}amino)ethyl]morpholine-4-carboxamide;
N-[2-(methyl{[methyl(phenyl)amino]carbonyl}amino)ethyl]-5-(phenylsulfonyl)-2-(trifluoromethyl)benzenesulfonamide;
N-Methyl-N-[2-({[5-(phenylsulfonyl)-2-(trifluoromethyl)phenyl]sulfonyl}amino)ethyl]-2-furamide;
4-tert-Butyl-N-methyl-N-[2-({[5-(phenylsulfonyl)-2-(trifluoromethyl)phenyl]sulfonyl}amino)ethyl]benzamide;
N-Methyl-N-[2-({[5-(phenylsulfonyl)-2-(trifluoromethyl)phenyl]sulfonyl}amino)ethyl]-2-(trifluoromethoxy)benzamide;
N-methyl-N-[2-({[5-(phenylsulfonyl)-2-(trifluoromethyl)phenyl]sulfonyl}amino)ethyl]cyclohexanecarboxamide;
3-Fluoro-N-methyl-N-[2-({[5-(phenylsulfonyl)-2-(trifluoromethyl)phenyl]sulfonyl}amino)ethyl]-4-(trifluoromethyl)benzamide;
Methyl 4-({methyl[2-({[5-(phenylsulfonyl)-2-(trifluoromethyl)phenyl]sulfonyl}amino)ethyl]amino carbonyl)benzoate;
N-Methyl-N-[2-({[5-(phenylsulfonyl)-2-(trifluoromethyl)phenyl]sulfonyl}amino)ethyl]nicotinamide;
N-Methyl-N-[2-({[5-(phenylsulfonyl)-2-(trifluoromethyl)phenyl]sulfonyl}amino)ethyl]isonicotinamide;
2-Chloro-N-methyl-N-[2-({[5-(phenylsulfonyl)-2-(trifluoromethyl)phenyl]sulfonyl}amino)ethyl]nicotinamide;
N,2,2-trimethyl-N-[2-({[5-(phenylsulfonyl)-2-(trifluoromethyl)phenyl]sulfonyl}amino)ethyl]propanamide;
2-ethyl-N-methyl-N-[2-({[5-(phenylsulfonyl)-2-(trifluoromethyl)phenyl]sulfonyl}amino)ethyl]butanamide;
butyl methyl[2-({[5-(phenylsulfonyl)-2-(trifluoromethyl)phenyl]sulfonyl}amino)ethyl]carbamate;
tert-butyl 4-[({methyl[2-({[5-(phenylsulfonyl)-2-(trifluoromethyl)phenyl]sulfonyl}amino)ethyl]amino }carbonyl)amino]piperidine-1-carboxylate;
2,2-dimethylpropyl methyl[2-({[5-(phenylsulfonyl)-2-(trifluoromethyl)phenyl]sulfonyl}amino)ethyl]carbamate;
isobutyl methyl[2-({[5-(phenylsulfonyl)-2-(trifluoromethyl)phenyl]sulfonyl}amino)ethyl]carbamate;
3-(trifluoromethyl)phenyl methyl[2-({[5-(phenylsulfonyl)-2-(trifluoromethyl)phenyl]sulfonyl}amino)ethyl]carbamate;
4-fluorophenyl methyl [2-({[5-(phenylsulfonyl)-2-(trifluoromethyl)phenyl]sulfonyl}amino)ethyl]carbamate;
4-bromophenyl methyl [2-({[5-(phenylsulfonyl)-2-(trifluoromethyl)phenyl]sulfonyl}amino)ethyl]carbamate;
N-(2-{methyl[(piperidin-4-ylamino)carbonyl]amino}ethyl)-5-(phenylsulfonyl)-2-(trifluoromethyl)benzenesulfonamide;
ethyl N-({methyl[2-({[5-(phenylsulfonyl)-2-(trifluoromethyl)phenyl]sulfonyl}amino)ethyl]amino}carbonyl)glycinate;
3-({methyl[2-({[5-(phenylsulfonyl)-2-(trifluoromethyl)phenyl]sulfonyl}amino)ethyl]amino}sulfonyl)benzoic acid;
tert-butyl 4-({methyl[2-({[5-(phenylsulfonyl)-2-(trifluoromethyl)phenyl]sulfonyl}amino)ethyl]amino }carbonyl)piperazine-1-carboxylate;
N-({methyl[2-({[5-(phenylsulfonyl)-2-(trifluoromethyl)phenyl]sulfonyl}amino)ethyl]amino}carbonyl)glycine;
4-{methyl [2-({[5-(phenylsulfonyl)-2-(trifluoromethyl)phenyl]sulfonyl}amino)ethyl]amino}-4-oxobutanoic acid;
N-methyl-N-[2-({[5-(phenylsulfonyl)-2-(trifluoromethyl)phenyl]sulfonyl}amino)ethyl]piperazine-1-carboxamide;
N-{2-[(2-hydroxyethyl)(methyl)amino]ethyl}-5-(phenylsulfonyl)-2-(trifluoromethyl)benzenesulfonamide;
methyl N-methyl-N-[2-({[5-(phenylsulfonyl)-2-(trifluoromethyl)phenyl]sulfonyl}amino)ethyl]glycinate;
ethyl N-methyl-N-[2-({[5-(phenylsulfonyl)-2-(trifluoromethyl)phenyl]sulfonyl}amino)ethyl]-□-alaninate;
tert-butyl (3S)-3-[({methyl[2-({[5-(phenylsulfonyl)-2-(trifluoromethyl)phenyl]sulfonyl}amino)ethyl]amino }carbonyl)amino]pyrrolidine-1-carboxylate;
tert-butyl methyl [3-({[5-(phenylsulfonyl)-2-(trifluoromethyl)phenyl]sulfonyl}amino)propyl]carbamate;
N-[3-(methylamino)propyl]-5-(phenylsulfonyl)-2-(trifluoromethyl)benzenesulfonamide;
N-[2-(methyl{[(3S)-pyrrolidin-3-ylamino]carbonyl}amino)ethyl]-5-(phenylsulfonyl)-2-(trifluoromethyl)benzenesulfonamide;
tert-butyl 4-[({methyl[3-({[5-(phenylsulfonyl)-2-(trifluoromethyl)phenyl]sulfonyl}amino)propyl]amino }carbonyl)amino]piperidine-1-carboxylate;
tert-butyl N-methyl-N-[2-({[5-(phenylsulfonyl)-2-(trifluoromethyl)phenyl]sulfonyl}amino)ethyl]-β-alaninate;
tert-butyl N-methyl-N-[2-({[5-(phenylsulfonyl)-2-(trifluoromethyl)phenyl]sulfonyl}amino)ethyl]glycinate;
N-(3-{methyl[(piperidin-4-ylamino)carbonyl]amino}propyl)-5-(phenylsulfonyl)-2-(trifluoromethyl)benzenesulfonamide;
tert-butyl 4-({methyl[3-({[5-(phenylsulfonyl)-2-(trifluoromethyl)phenyl]sulfonyl}amino)propyl]amino }carbonyl)piperazine-1-carboxylate;
N-methyl-N-[3-({[5-(phenylsulfonyl)-2-(trifluoromethyl)phenyl]sulfonyl}amino)propyl]piperazine-1-carboxamide;
4-{4-[({methyl[2-({[5-(phenylsulfonyl)-2-(trifluoromethyl)phenyl]sulfonyl}amino)ethyl]amino}carbonyl)amino]piperidin-1-yl}-4-oxobutanoic acid;
N-methyl-N-[2-({[5-(phenylsulfonyl)-2-(trifluoromethyl)phenyl]sulfonyl}amino)ethyl]glycine;
N-methyl-N-[2-({[5-(phenylsulfonyl)-2-(trifluoromethyl)phenyl]sulfonyl}amino)ethyl]-β-alanine;
4-(bromomethyl)-N-methyl-N-[2-({[5-(phenylsulfonyl)-2-(trifluoromethyl)phenyl]sulfonyl}amino)ethyl]benzamide;
tert-butyl [3-({[5-(phenylsulfonyl)-2-(trifluoromethyl)phenyl]sulfonyl}amino)propyl]carbamate;
tert-butyl 4-[methyl({methyl[2-({[5-(phenylsulfonyl)-2-(trifluoromethyl)phenyl]sulfonyl}amino)ethyl]amino}carbonyl)amino]piperidine-1-carboxylate;
tert-butyl (3R)-3-[({methyl[2-({[5-(phenylsulfonyl)-2-(trifluoromethyl)phenyl]sulfonyl}amino)ethyl]amino}carbonyl)amino]pyrrolidine-1-carboxylate;
4-{methyl[3-({[5-(phenylsulfonyl)-2-(trifluoromethyl)phenyl]sulfonyl}amino)propyl]amino}-4-oxobutanoic acid;
N-(3-aminopropyl)-5-(phenylsulfonyl)-2-(trifluoromethyl)benzenesulfonamide;
dimethyl [4-({methyl [2-({[5-(phenylsulfonyl)-2-(trifluoromethyl)phenyl]sulfonyl}amino)ethyl]amino}carbonyl)benzyl]phosphonate;
N-[2-(methyl{[methyl(piperidin-4-yl)amino]carbonyl}amino)ethyl]-5-(phenylsulfonyl)-2-(trifluoromethyl)benzenesulfonamide;
N-[2-(methyl{[(3R)-pyrrolidin-3-ylamino]carbonyl}amino)ethyl]-5-(phenylsulfonyl)-2-(trifluoromethyl)benzenesulfonamide;
tert-butyl (3R)-3-[({methyl[2-({[5-(phenylsulfonyl)-2-(trifluoromethyl)phenyl]sulfonyl}amino)ethyl]amino}carbonyl)amino]piperidine-1-carboxylate;
tert-butyl (3S)-3-[({methyl[2-({[5-(phenylsulfonyl)-2-(trifluoromethyl)phenyl]sulfonyl}amino)ethyl]amino]carbonyl)amino]piperidine-1-carboxylate;
N-[2-(methyl{[(3R)-piperidin-3-ylamino]carbonyl}amino)ethyl]-5-(phenylsulfonyl)-2-(trifluoromethyl)benzenesulfonamide;
N-[2-(methyl{[(3S)-piperidin-3-ylamino]carbonyl}amino)ethyl]-5-(phenylsulfonyl)-2-(trifluoromethyl)benzenesulfonamide;
[4-({methyl[2-({[5-(phenylsulfonyl)-2-(trifluoromethyl)phenyl]sulfonyl}amino)ethyl]amino}carbonyl)benzyl]phosphonic acid;
tert-butyl 4-[methyl({[2-({[5-(phenylsulfonyl)-2-(trifluoromethyl)phenyl]sulfonyl}amino)ethyl]amino}carbonyl)amino]piperidine-1-carboxylate;
tert-butyl 4-[({[2-({[5-(phenylsulfonyl)-2-(trifluoromethyl)phenyl]sulfonyl}amino)ethyl]amino}carbonyl)amino]piperidine-1-carboxylate;
N-(2,3-dihydro-1H-inden-2-yl)-5-(phenylsulfonyl)-2-(trifluoromethyl)benzenesulfonamide;
N-(2-hydroxy-2,3-dihydro-1H-inden-1-yl)-5-(phenylsulfonyl)-2-(trifluoromethyl)benzenesulfonamide;
N-(1-hydroxy-2,3-dihydro-1H-inden-2-yl)-5-(phenylsulfonyl)-2-(trifluoromethyl)benzenesulfonamide;
N-(5-methoxy-2,3-dihydro-1H-inden-2-yl)-5-(phenylsulfonyl)-2-(trifluoromethyl)benzenesulfonamide;
N-(5-hydroxy-2,3-dihydro-1H-inden-2-yl)-5-(phenylsulfonyl)-2-(trifluoromethyl)benzenesulfonamide;
methyl {[2-({[5-(phenylsulfonyl)-2-(trifluoromethyl)phenyl]sulfonyl}amino)-2,3-dihydro-1H -inden-5-yl]oxy }acetate;
{[2-({[5-(phenylsulfonyl)-2-(trifluoromethyl)phenyl]sulfonyl}amino)-2,3-dihydro-1H-inden-5-yl]oxy}acetic acid;
methyl 2-({[5-(phenylsulfonyl)-2-(trifluoromethyl)phenyl]sulfonyl}amino)indane-5-carboxylate;
N-(1-hydroxy-6-methoxy-2,3-dihydro-1H-inden-2-yl)-5-(phenylsulfonyl)-2-(trifluoromethyl)benzenesulfonamide;
2-{2-({[5-(phenylsulfonyl)-2-(trifluoromethyl)phenyl]sulfonyl}amino)-2,3-dihydro-1H-inden-5-yl]oxy}acetamide;
2-({[5-(phenylsulfonyl)-2-(trifluoromethyl)phenyl]sulfonyl}amino)indane-5-carboxylic acid; or
N-(5-bromo-2,3-dihydro-1H-inden-2-yl)-5-(phenylsulfonyl)-2-(trifluoromethyl)benzenesulfonamide.

In certain embodiments of the invention, when R₁ of Formula I is phenyl; X of Formula I is O; R₂ of Formula I is CH₃; and R₄, R₅, R₆, and R₇ of Formula I are each H; then R₃ of Formula I is not methylphenyl, ethylphenyl, or hydrogen. In other embodiments, when R₁ of Formula I is phenyl; X of Formula I is O; R₂ of Formula I is CH₃; and R₄, R₅, R₆, and R₇ of Formula I are each H; then R₃ of Formula I is not alkylphenyl or hydrogen. In still further embodiments, when R₁ of Formula I is phenyl; X of Formula I is O; R₂ of Formula I is CH₃; and R₄, R₅, R₆, and R₇ of Formula I are each H; then R₃ of Formula I is not alkylaryl or hydrogen.

In further embodiments of the invention, when R₁ of Formula I is chlorophenyl; X of Formula I is O; R₂ of Formula I is CH₃; and R₄, R₅, R₆, and R₇ of Formula I are each H; then R₃ of Formula I is not cyclohexyl, methylphenyl, methylfuranyl, methylpyridyl, or hydrogen. In other embodiments, when R₁ of Formula I is chlorophenyl; X of Formula I is O; R₂ of Formula I is CH₃; and R₄, R₅, R₆, and R₇ of Formula I are each H; then R₃ of Formula I is not cycloalkyl, alkylphenyl, alkylfuranyl, alkylpyridyl, or hydrogen. In additional embodiments, when R₁ of Formula I is chlorophenyl; X of Formula I is O; R₂ of Formula I is CH₃; and R₄, R₅, R₆, and R₇ of Formula I are each H; then R₃ of Formula I is not cycloalkyl, alkylaryl, alkylheteroaryl, or hydrogen.

In certain embodiments of the invention, when R₁ of Formula I is bromophenyl; X of Formula I is O; R₂ of Formula I is CH₃; and R₄, R₅, R₆, and R₇ of Formula I are each H; then R₃ of Formula I is not hydrogen.

In certain other embodiments of the invention, when R₁ of Formula I is nitrophenyl or dinitrophenyl; X of Formula I is O; R₂ of Formula I is CH₃; and R₄, R₅, R₆, and R₇ of Formula I are each H; then R₃ of Formula I is not hydrogen.

Compounds of Formula 1 may be used to modulate the activity of secreted frizzled related protein-1. Such compounds are of interest for the treatment of bone fractures as well as bone disorders, including osteoporosis, and for the treatment of arthritis, chronic obstructive pulmonary disease, cartilage defects, prostate cancer and leiomyoma.

In certain embodiments, the present invention therefore provides methods of treating, preventing, inhibiting, or alleviating each of the maladies listed above in a mammal, preferably in a human, comprising administering a therapeutically effective amount of a compound of Formula 1 or a pharmaceutically acceptable salt thereof to a patient suspected to suffer from such a malady.

In other embodiments, the invention relates to compositions comprising at least one compound of Formula 1, or a steroisomer or pharmaceutically acceptable salt thereof, and one or more pharmaceutically acceptable carriers, excipients, or diluents. Such compositions include pharmaceutical compositions for treating or controlling disease states or conditions of the bone. In certain embodiments, the compositions comprise mixtures of one or more compounds of Formula 1.

Certain of the compounds of Formula 1 contain stereogenic carbon atoms or other chiral elements and thus give rise to stereoisomers, including enantiomers and diastereomers. The invention generally relates to all stereoisomers of the compounds of Formula 1, as well as to mixtures of the stereoisomers. Throughout this application, the name of a compound without indication as to the absolute configuration of an asymmetric center is intended to embrace the individual stereoisomers as well as mixtures of stereoisomers. Reference to optical rotation [(+), (—) and (±)]is utilized to distinguish the enantiomers from one another and from the racemate. Furthermore, throughout this application, the designations R* and S* are used to indicate relative stereochemistry, employing the Chemical Abstracts convention which automatically assigns R* to the lowest numbered asymmetric center.

An enantiomer can, in some embodiments of the invention, be provided substantially free of the corresponding enantiomer. Thus, reference to an enantiomer as being substantially free of the corresponding enantiomer indicates that it is isolated or separated via separation techniques or prepared so as to be substantially free of the corresponding enantiomer. “Substantially free,” as used herein, means that a significantly lesser proportion of the corresponding enantiomer is present. In preferred embodiments, less than about 90% by weight of the corresponding enantiomer is present relative to desired enantiomer, more preferably less than about 1% by weight. Preferred enantiomers can be isolated from racemic mixtures by any method known to those skilled in the art, including high performance liquid chromatography (HPLC), and the formation and crystallization of chiral salts, or preferred enantiomers, can be prepared by methods described herein. Methods for the preparation of enantiomers are described, for example, in Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, N.Y., 1962); and Wilen, S. H. Tables of Resolving Agents and Optical Resolutions p. 268 (E. L. Eliel, Ed., Univ. of Notre Dame Press, Notre Dame, Ind. 1972), each of which is hereby incorporated by reference in its entirety.

The following synthetic schemes are designed to illustrate, but not limit, general procedures for the preparation of compounds of Formula 1. The reagents used can be either commercially obtained or can be prepared by standard procedures described in the literature.

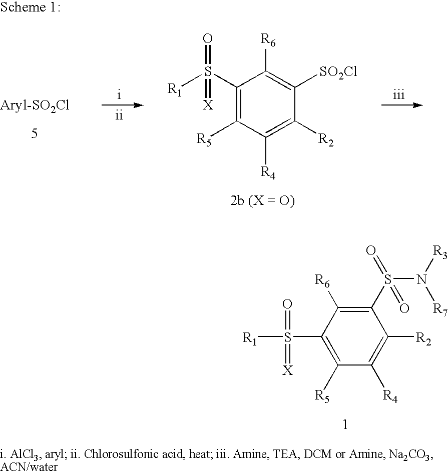

In Scheme 1, step i, a suitably substituted aryl sulfonyl chloride (5), either commercially available, known in the literature, or prepared according to methods known and established for the preparation of sulfonyl chlorides, including procedures exemplified in the experimental section of this document, wherein, R₂, R₄, R₅, R₆ and R₁ are as previously defined, is reacted with an unsubstituted or suitably substituted aryl group, either commercially available, known in the literature, or prepared according to methods known and established for the preparation of substituted benzenes, with or without a Lewis acid catalyst such as aluminum chloride, either using the substituted aryl in excess as the solvent, or using another suitably acceptable solvent. The diaryl sulfone product is then treated with chlorosulfonic acid with or without solvent, step ii, at room temperature or with heating for several hours or longer where appropriate to provide a sulfonyl chloride (2b), which is used directly or purified according to established procedures. The sulfonyl chloride (2) is reacted in step iii with an amine (R₇R₃NH), either commercially available, known in the literature, or prepared according to methods known and established for the preparation of primary and secondary amines, in a suitable solvent, such as dichloromethane or acetonitrile, in the presence of an acid scavenger, such as triethylamine, at room temperature to afford the desired product (1).

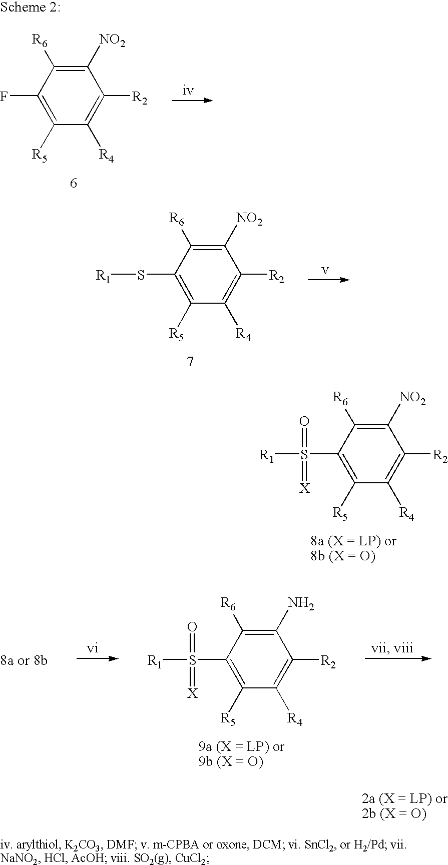

In Scheme 2, step iv, a suitably substituted 3-fluoro nitrobenzene derivative, either commercially available, known in the literature, or prepared according to methods known and established for the preparation of such nitrobenzenes, is reacted with an appropriately substituted arylthiol, either commercially available, known in the literature, or prepared according to methods known and established for the preparation of such arylthiols, in the presence of an acid scavenger, such as potassium carbonate, in a suitable solvent, such as dimethylformamide or dimethylacetamide, at an elevated temperature for several hours to provide diaryl sulfide (7). The diaryl sulfide (7) is oxidized, step v, using established procedures, or as known in the literature, in an acceptable solvent such as dichloromethane, with a suitable oxidant, such as meta-chloroperoxybenzoic acid or oxone, to afford either the sulfoxide (8a) or sulfone (8b). The sulfoxide or sulfone is subjected to reducing conditions, such as stannous chloride, step vi, or reagents that are commonly used to effect the reduction of a nitro group, known in the literature, to afford aniline (9a or 9b). Diazotization, step vii, of the anilinium hydrochloride salt under acidic conditions with sodium nitrite, followed by sulfonylation, step viii, with sulfur dioxide and hydrogen chloride in the presence of an organic metal catalyst, such as copper (II) chloride, according to procedures described in the literature, results in the formation of sulfonyl chloride (2), which may be transformed into the sulfonamide (1) as previously described, step iii.

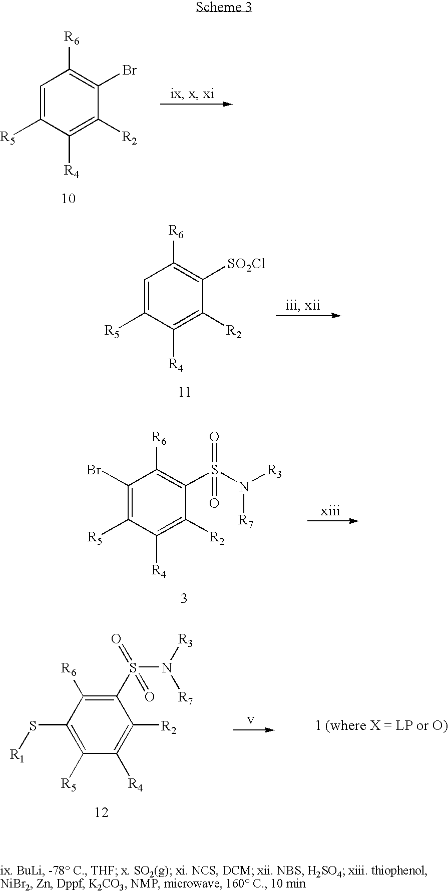

In Scheme 3, an appropriately substituted sulfonyl chloride (11), wherein R₂ is as previously defined for alkyl, either commercially available, known in the literature, or prepared according to methods known and established for the preparation of such sulfonyl chlorides, including procedures exemplified in the experimental section, such as from aryl bromide (10), either commercially available, known in the literature, or prepared according to methods known and established for the preparation of such aryl bromides, is transmetalated, step ix, at reduced temperature and in a suitable solvent such as tetrahydrofuran, using an acceptable organometallic reagent that is known to effect such transformations, such as n-butyl lithium, and the resulting metalated species is treated with sulfur dioxide, step x, to afford a sulfinic acid intermediate that is isolated or immediately oxidized, step xi, to a sulfonyl chloride (11). The sulfonyl chloride (11) is then transformed into a sulfonamide, step iii, and brominated, step xii, using N-bromosuccinimide in concentrated sulfuric acid, or other brominating conditions that are commonly used to brominate aryl sulfonamides, to afford an aryl bromosulfonamide (3). The aryl bromosulfonamide (3) is then reacted with an arylthiol, either commercially available, known in the literature, or prepared according to methods known and established for the preparation of thiophenols, using a catalytic system comprised of nickel (II) bromide and zinc metal, with a suitable ligand such as Dppf, step xiii, in the presence of an inorganic base, such as potassium carbonate, and a polar aprotic solvent, such as 1-methyl-2-pyrrolidinone, at elevated temperatures to afford a sulfide (12), which is oxidized, step v, using a suitable oxidant such as meta-chloroperoxybenzoic acid or oxone, or by established procedures, as known in the literature, in an acceptable solvent, such as dichloromethane, for the oxidation of sulfides, including procedures exemplified in the experimental section, to afford a diarylsulfone sulfonamide (1).

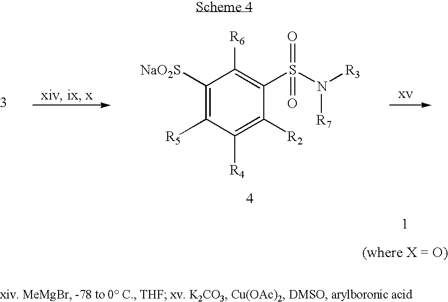

Alternatively, as depicted in Scheme 4, intermediate 3 is exposed to methyl magnesium bromide, step xiv, followed by a metal halogen exchange reagent, such as butyl lithium, step ix, at reduced temperature and in a suitable solvent, such as tetrahydrofuran, to afford a lithiated species that is quenched with sulfur dioxide, step x, to afford a sulfinic acid that is isolated as the sodium salt (4), as exemplified in the experimental section. The sodium sulfinate (4) is then reacted with an appropriately substituted aryl boronic acid, step xv, either commercially available, known in the literature, or prepared according to methods known and established for the preparation of aryl boronic acids, in a cross coupling reaction that is promoted by a copper salt, such as copper (II) acetate, in an appropriate polar aprotic solvent, such as dimethylsulfoxide or dimethylforamide, in the presence of an acid scavenger, such as triethylamine or potassium carbonate, and a desiccant, such as molecular sieves, at ambient or elevated temperatures, or as exemplified in the experimental section, to provide compounds of Formula 1.

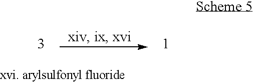

Alternatively, as depicted in Scheme 5, the lithiated intermediate from 3, Scheme 4, is quenched with an appropriately substituted aryl sulfonyl fluoride, step xvi, either commercially available, known in the literature, or prepared according to methods known and established for the preparation of aryl sulfonyl fluorides, as exemplified in the experimental section, to provide compounds of Formula 1.

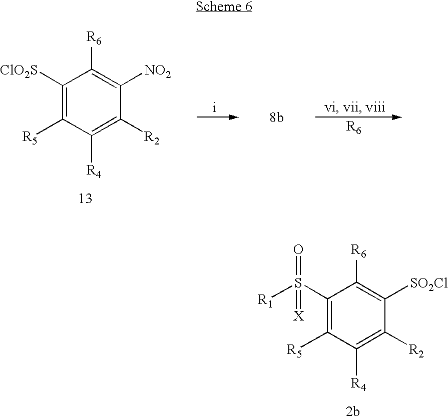

In Scheme 6, an appropriately substituted aryl sulfonyl chloride, either commercially available, known in the literature, or prepared according to methods known and established for the preparation of aryl sulfonyl chlorides, is elaborated as described in Scheme 1, step i, to provide 8, and then, according to Scheme 2, steps vi, vii and viii, to provide sulfonyl chloride (2), which is transformed to compounds of Formula 1 as previously described.

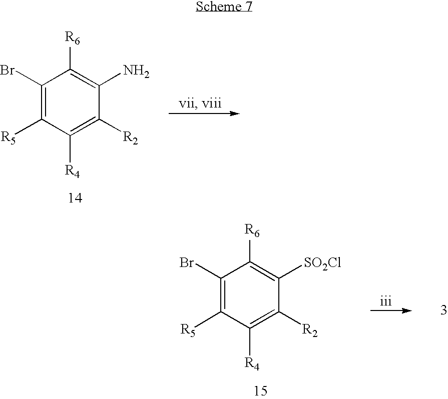

Alternatively, as depicted in Scheme 7, intermediate 3 is prepared from an appropriately substituted bromoaniline (14), either commercially available, known in the literature, or prepared according to methods known and established for the preparation of bromoanilines, according to the protocol followed in Scheme 2, steps vii and viii, to afford a sulfonyl chloride (15), which is converted into a sulfonamide (3), step iii, as outlined in the previous schemes. Intermediate 3 is elaborated to compounds of Formula 1 using methods previously described in Schemes 3, 4 or 5, as appropriate.

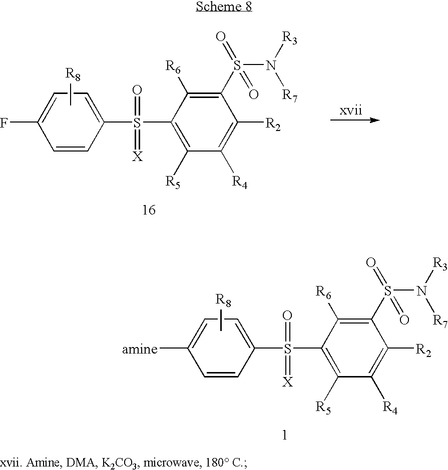

In Scheme 8, an aryl fluoride (16), either prepared according to procedures previously described, exemplified in the experimental section, commercially available, or known in the literature, is reacted with an appropriately substituted primary or secondary amine, either commercially available or known in the literature, using a suitable polar solvent, such as dimethylacetamide, in the presence of an acid scavenger, such as potassium carbonate, at elevated temperatures to afford the desired product.

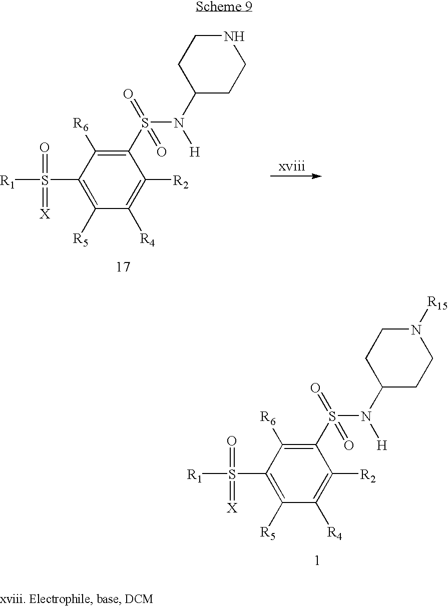

The piperidinyl sulfonamide (17), shown in Scheme 9, either prepared according to procedures exemplified in the experimental section, commercially available, or known in the literature, is reacted with an appropriately substituted electrophile such as, but not limited to, an acid chloride, sulfonyl chloride, alkyl halide, isothiocyanate, isocyanate, or epoxide, in the presence of an acid scavenger, such as morpholinomethyl-polystyrene or triethylamine where appropriate, in a suitable solvent, such as dichloromethane, to provide the desired product of Formula 1.

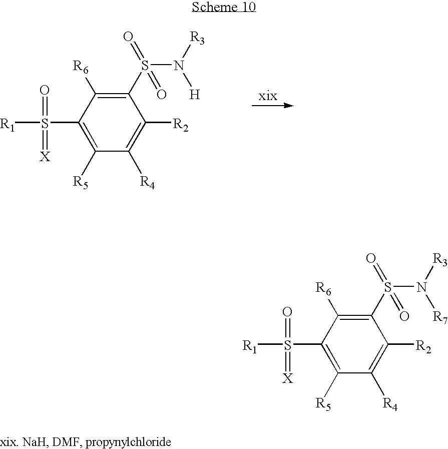

Compounds of Formula 1, in which R₇ is H and R₃ is as previously described, are further alkylated by deprotonating using an alkali base, such as sodium hydride, in an appropriate solvent, such as dimethylformamide, and subsequently treating with an alkylating agent, such as propynyl chloride, to yield a tertiary sulfonamide.

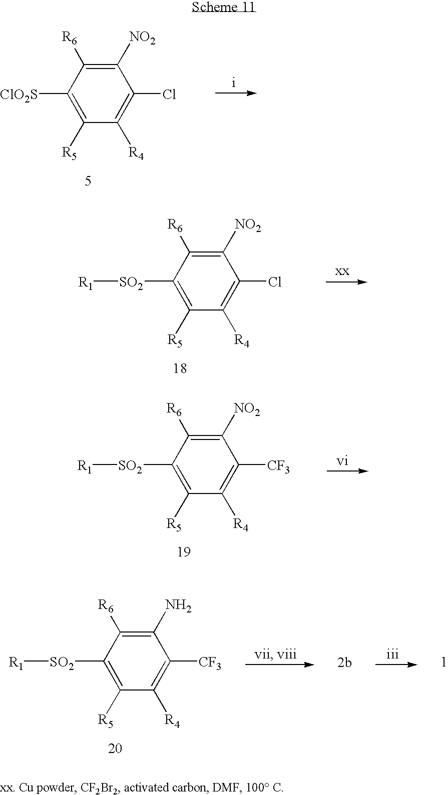

In Scheme 11, an aryl sulfonyl chloride (5), either prepared according to procedures previously described, commercially available, or known in the literature, is reacted as described in Scheme 1, step i, to afford an intermediate diarylsulfone (18). Reaction of 18 with an in situ derived copper trifluoromethylating agent, step xx, also known in the literature as Burton's reagent, which is generated by the reaction of copper metal and dibromodifluoromethane in a polar aprotic solvent such as dimethylformamide, and heating for several hours, provides the trifluoromethyl intermediate 19. The conversion of 19 to aniline 20 and then to the subsequent sulfonyl chloride proceeds as previously described using steps vi-viii.

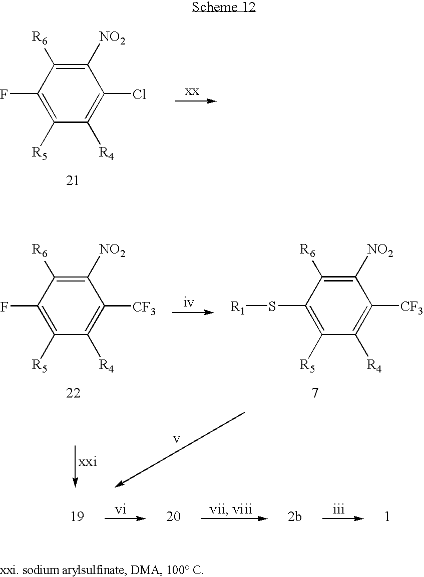

Alternatively, in Scheme 12, the nitro benzene (21) can be converted to the trifluoromethyl intermediate (22) in an analogous manner used to prepared 19, employing step xx. Further elaboration of 22 to a diaryl sulfide (7), step iv, in an analogous fashion as outlined in Scheme 2. The sulfide can than be oxidized to the diaryl sulfone (19) using procedures that were previously discussed in Scheme 2, step v. Diaryl sulfone (19) can also be directly prepared by reacting 22 with an aryl sulfinate, step xxi, either commercially available, or known in the literature, in a polar aprotic solvent such as dimethylacetamide with heating for several hours. Intermediate 19 can be further transformed to aniline 20, step vi, using previously established procedures. The aniline (20) can then be converted into the sulfonyl chloride, step vii-viii, using previously established procedures, and subsequently reacted with an amine, step iii, to afford compounds of formula 1.

In certain embodiments, the invention relates to compositions comprising at least one compound of Formula 1, or a stereoisomer or pharmaceutically acceptable salt thereof, and one or more pharmaceutically acceptable carriers, excipients, or diluents. Such compositions are prepared in accordance with general pharmaceutical formulation procedures, such as, for example, those described in Remingtons Pharmaceutical Sciences, 17th edition, ed. Alfonoso R. Gennaro, Mack Publishing Company, Easton, Pa. (1985), which is incorporated herein by reference in its entirety. Pharmaceutically acceptable carriers are those carriers that are compatible with the other ingredients in the formulation and are biologically acceptable.

The compounds of Formula 1 can be administered orally or parenterally, neat, or in combination with conventional pharmaceutical carriers. Applicable solid carriers can include one or more substances that can also act as flavoring agents, lubricants, solubilizers, suspending agents, fillers, glidants, compression aids, binders, tablet-disintegrating agents, or encapsulating materials. In powders, the carrier is a finely divided solid that is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Liquid carriers can be used in preparing solutions, suspensions, emulsions, syrups and elixirs. The active ingredient can be dissolved or suspended in a pharmaceutically acceptable liquid carrier such as water, an organic solvent, a mixture of both, or a pharmaceutically acceptable oil or fat. The liquid carrier can contain other suitable pharmaceutical additives such as, for example, solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (particularly containing additives as above, e.g. cellulose derivatives, preferably sodium carboxymethyl cellulose solution), alcohols (including monohydric alcohols and polyhydric alcohols e.g. glycols) and their derivatives, and oils (e.g. fractionated coconut oil and arachis oil). For parenteral administration, the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

Liquid pharmaceutical compositions that are sterile solutions or suspensions can be administered by, for example, intramuscular, intraperitoneal or subcutaneous injection. Sterile solutions can also be administered intravenously. Compositions for oral administration can be in either liquid or solid form.

The compounds of Formula 1 can be administered rectally or vaginally in the form of a conventional suppository. For administration by intranasal or intrabronchial inhalation or insufflation, the compounds of Formula 1 can be formulated into an aqueous or partially aqueous solution, which can then be utilized in the form of an aerosol. The compounds of Formula 1 can also be administered transdermally through the use of a transdermal patch containing the active compound and a carrier that is inert to the active compound, is non-toxic to the skin, and allows delivery of the agent for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the active ingredient can also be suitable. A variety of occlusive devices can be used to release the active ingredient into the blood stream such as a semipermeable membrane covering a reservoir containing the active ingredient with or without a carrier, or a matrix containing the active ingredient. Other occlusive devices are known in the literature.

Preferably the pharmaceutical composition is in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example, packeted powders, vials, ampoules, prefilled syringes or sachets containing liquids. The unit dosage form can be, for example, a capsule or tablet itself, or it can be the appropriate number of any such compositions in package form.

The amount provided to a patient will vary depending upon what is being administered, the purpose of the administration, such as prophylaxis or therapy, and the state of the patient, the manner of administration, and the like. In therapeutic applications, compounds of Formula 1 are provided to a patient already suffering from a disease in an amount sufficient to cure or at least partially ameliorate the symptoms of the disease and its complications. An amount adequate to accomplish this is defined as a “therapeutically effective amount.” The dosage to be used in the treatment of a specific case must be subjectively determined by the attending physician. The variables involved include the specific condition and the size, age, and response pattern of the patient. The compounds can be administered orally, rectally, parenterally, or topically to the skin and mucosa. The usual daily dose depends on the specific compound, method of treatment and condition treated. The usual daily dose is 0.01-1000 mg/kg for oral application, preferably 0.5-500 mg/kg, and 0.1-100 mg/kg for parenteral application, preferably 0.5-50 mg/kg.

In certain embodiments, the present invention is directed to prodrugs of compounds of Formula 1. The term “prodrug,” as used herein, means a compound that is convertible in vivo by metabolic means (e.g. by hydrolysis) to a compound of Formula 1. Various forms of prodrugs are known in the art such as those discussed in, for example, Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991), Bundgaard, et al., Journal of Drug Delivery Reviews, 8:1-38(1992), Bundgaard, J. of Pharmaceutical Sciences, 77:285 et seq. (1988); and Higuchi and Stella (eds.) Prodrugs as Novel Drug Delivery Systems, American Chemical Society (1975), each of which is hereby incorporated by reference in its entirety.

The following examples are illustrative of certain embodiments of the invention and should not be considered to limit the scope of the invention. ACD NamePro software was employed to generate IUPAC names for the following examples. The IUPAC names of the following examples are indicative of the neutral or free base forms. Compounds were either isolated as a free base or the corresponding hydrochloride salt as indicated in the experimental procedure.

### EXAMPLE 1

### 2-Methyl-N-(2-phenylethyl)-5-(phenylsulfonyl)benzene sulfonamide

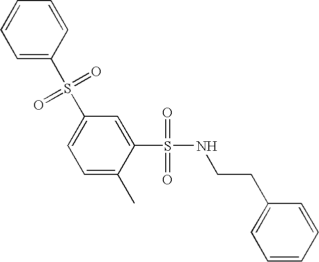

Step 1: To phenyl-4-tolyl sulfone (2.32 g, 10.0 mmol) was added chloro sulfonic acid (6.7 mL, 100 mmol) and the reaction mixture was stirred at 50° C. for 5 hours and then cooled to room temperature. The reaction mixture was slowly poured into ice (200 g) and a white solid precipitated. The resulting suspension was extracted with ethyl acetate (150 mL×3) and the organic layers were combined and washed with brine (200 mL) and collected. The collected organic layer was dried over sodium sulfate and concentrated to give 5-Benzenesulfonyl-2-methyl-benzenesulfonyl chloride (3.30 g, 100% yield). The product was used without further purification.

Step 2: The mixture of 5-Benzenesulfonyl-2-methyl-benzenesulfonyl chloride (66 mg, 0.20 mmol), phenethylamine (36 mg, 0.30 mmol) and triethyl amine (30 mg, 0.30 mmol) in methylene chloride (2 mL) was stirred at 37° C. for 12 hours. Then the reaction mixture was concentrated and purified by reverse phase chromatography to give 2-Methyl-N-(2-phenylethyl)-5-(phenylsulfonyl) benzene sulfonamide (68 mg, 82% yield).

LC/MS conditions: Aquasil C18; Mobile Phase A: 100% (0.1% Formic Acid) in water (by volume), B: 100% (0.1% Formic Acid) in CAN; Flow Rate: 0.800 mL/min.

Column Temperature: 40° C.; Injection Volume:5 μL, LV: monitor 215, 230, 254, 280, and 300 nm; Purity is reported at 254 nm unless otherwise noted.

Gradient Table:

| Time (min) | % B |
|---|---|
| 0 | 0 |
| 2.5 | 100 |
| 4.0 | 100 |
| 4.1 | 0 |
| 5.5 | 0 |

MS Conditions (Same for both LC conditions)

| | |
|---|---|
| Instrument: | Agilent MSD |
| Ionization Mode: | API-ES |
| Gas Temperature: | 350° C. |
| Drying Gas: | 13.0 L/min. |
| Nebulizer Pressure: | 55 psig |
| Polarity: | 50% positive, 50% negative |
| VCap: | 3000 V (positive), 2500 V (negative) |
| Fragmentor: | 120 (positive), 120 (negative) |
| Mass Range: | 100-1000 m/z |
| Threshold: | 150 |
| Step size: | 0.15 |
| Gain: | 1 |
| Peak width: | 0.15 min |

HPLC: Rt = 3.1 min; MS 416(M+H).

¹HNMR (CDCl₃) δ 8.48 (1H, d), 7.97 (1H, dd), 7.94 (2H, dd), 7.58 (dd, 1H), 7.51 (2H, dd), 7.39 (1H, dd), 7.30 (2H, dd), 7.05 (2H, dd), 4.44 (1H, t), 3.27 (2H, m), 2.78 (2H, t), 2.46 (3H, s).

### EXAMPLES 2 TO 30

The compounds of Examples 2 to 30 were prepared generally according to the procedures described in Example 1.

| | | Prepared |
|---|---|---|
| | | according to |
| | | description |
| Example | | provided in |
| No. | Compound Name | Example No. |
| 2 | 5-[(4-fluorophenyl)sulfonyl]-2-methyl-N-(2- | 1 |
| | phenylethyl)benzenesulfonamide | |
| 3 | N-[2-(2-chlorophenyl)ethyl]-5-[(4-fluorophenyl)sulfonyl]-2- | 1 |
| | methylbenzenesulfonamide | |
| 4 | 5-[(4-fluorophenyl)sulfonyl]-N-[2-(2-methoxyphenyl)ethyl]-2- | 1 |
| | methylbenzenesulfonamide | |
| 5 | 5-[(4-fluorophenyl)sulfonyl]-N-[2-(3-methoxyphenyl)ethyl]-2- | 1 |
| | methylbenzenesulfonamide | |
| 6 | N-[2-(3,4-dimethoxyphenyl)ethyl]-5-[(4-fluorophenyl)sulfonyl]-2- | 1 |
| | methylbenzenesulfonamide | |
| 7 | N-[2-(4-bromophenyl)ethyl]-5-[(4-fluorophenyl)sulfonyl]-2- | 1 |
| | methylbenzenesulfonamide | |
| 8 | N-[2-(4-fluorophenyl)ethyl]-5-[(4-fluorophenyl)sulfonyl]-2- | 1 |
| | methylbenzenesulfonamide | |
| 9 | N-[2-(4-chlorophenyl)ethyl]-5-[(4-fluorophenyl)sulfonyl]-2- | 1 |
| | methylbenzenesulfonamide | |
| 10 | 5-[(4-fluorophenyl)sulfonyl]-N-[2-(4-methoxyphenyl)ethyl]-2- | 1 |
| | methylbenzenesulfonamide | |
| 11 | 5-[(4-fluorophenyl)sulfonyl]-2-methyl-N-[2-(4- | 1 |
| | methylphenyl)ethyl]benzenesulfonamide | |
| 12 | N-{2-[4-(aminosulfonyl)phenyl]ethyl}-5-[(4-fluorophenyl)sulfonyl]- | 1 |
| | 2-methylbenzenesulfonamide | |
| 13 | N-{2-[3,4-bis(benzyloxy)phenyl]ethyl}-5-[(4-fluorophenyl)sulfonyl]- | 1 |
| | 2-methylbenzenesulfonamide | |
| 14 | 5-[(4-fluorophenyl)sulfonyl]-2-methyl-N-[2-(4- | 1 |
| | nitrophenyl)ethyl]benzenesulfonamide | |
| 15 | N-[2-(1,3-benzodioxol-5-yl)ethyl]-5-[(4-fluorophenyl)sulfonyl]-2- | 1 |
| | methylbenzenesulfonamide | |
| 16 | 5-[(4-fluorophenyl)sulfonyl]-2-methyl-N-{2-[3- | 1 |
| | (trifluoromethyl)phenyl]ethyl}benzenesulfonamide | |
| 17 | N-[2-(3-chlorophenyl)ethyl]-5-[(4-fluorophenyl)sulfonyl]-2- | 1 |
| | methylbenzenesulfonamide | |
| 18 | N-[2-(2,4-dichlorophenyl)ethyl]-5-[(4-fluorophenyl)sulfonyl]-2- | 1 |
| | methylbenzenesulfonamide | |
| 19 | N-[2-(2,6-dichlorophenyl)ethyl]-5-[(4-fluorophenyl)sulfonyl]-2- | 1 |
| | methylbenzenesulfonamide | |
| 20 | N-[2-(2,5-dimethoxyphenyl)ethyl]-5-[(4-fluorophenyl)sulfonyl]-2- | 1 |
| | methylbenzenesulfonamid | |
| 21 | N-[2-(3,4-dichlorophenyl)ethyl]-5-[(4-fluorophenyl)sulfonyl]-2- | 1 |
| | methylbenzenesulfonamide | |
| 22 | N-[2-(3,5-dimethoxyphenyl)ethyl]-5-[(4-fluorophenyl)sulfonyl]-2- | 1 |
| | methylbenzenesulfonamide | |
| 23 | N-[2-(4-ethoxyphenyl)ethyl]-5-[(4-fluorophenyl)sulfonyl]-2- | 1 |
| | methylbenzenesulfonamide | |
| 24 | N-[2-(3-fluorophenyl)ethyl]-5-[(4-fluorophenyl)sulfonyl]-2- | 1 |
| | methylbenzenesulfonamide | |
| 25 | N-[2-(2-fluorophenyl)ethyl]-5-[(4-fluorophenyl)sulfonyl]-2- | 1 |
| | methylbenzenesulfonamide | |
| 26 | N-[2-(3-ethoxy-4-methoxyphenyl)ethyl]-5-[(4-fluorophenyl)sulfonyl]- | 1 |
| | 2-methylbenzenesulfonamide | |
| 27 | N-[2-(4-ethoxy-3-methoxyphenyl)ethyl]-5-[(4-fluorophenyl)sulfonyl]- | 1 |
| | 2-methylbenzenesulfonamide | |
| 28 | 5-[(4-fluorophenyl)sulfonyl]-N-(4-methoxybenzyl)-2- | 1 |
| | methylbenzenesulfonamide | |
| 29 | N-[2-(2-bromo-4,5-dimethoxyphenyl)ethyl]-5-[(4- | 1 |
| | fluorophenyl)sulfonyl]-2-methylbenzenesulfonamide | |
| 30 | N-[2-(5-bromo-2-methoxyphenyl)ethyl]-5-[(4-fluorophenyl)sulfonyl]- | 1 |
| | 2-methylbenzenesulfonamide | |

### EXAMPLES 31 TO 179

The compounds of Examples 31 to 179 were prepared generally according to the procedures described in Example 1.

| | | Prepared |
|---|---|---|
| | | according |
| | | description |
| Example | | provided in |
| No. | Compound Name | Example No. |
| 31 | N-[2-(2-chlorophenyl)ethyl]-2-methyl-5- | 1 |
| | (phenylsulfonyl)benzenesulfonamide | |
| 32 | N-[2-(2-methoxyphenyl)ethyl]-2-methyl-5- | 1 |
| | (phenylsulfonyl)benzenesulfonamide | |
| 33 | N-[2-(3-methoxyphenyl)ethyl]-2-methyl-5- | 1 |
| | (phenylsulfonyl)benzenesulfonamide | |
| 34 | N-[2-(3,4-dimethoxyphenyl)ethyl]-2-methyl-5- | 1 |
| | (phenylsulfonyl)benzenesulfonamide | |
| 35 | N-[2-(4-bromophenyl)ethyl]-2-methyl-5- | 1 |
| | (phenylsulfonyl)benzenesulfonamide | |
| 36 | N-[2-(4-fluorophenyl)ethyl]-2-methyl-5- | 1 |
| | (phenylsulfonyl)benzenesulfonamide | |
| 37 | N-[2-(4-chlorophenyl)ethyl]-2-methyl-5- | 1 |
| | (phenylsulfonyl)benzenesulfonamide | |
| 38 | N-[2-(4-methoxyphenyl)ethyl]-2-methyl-5- | 1 |
| | (phenylsulfonyl)benzenesulfonamide | |
| 39 | 2-methyl-N-[2-(4-methylphenyl)ethyl]-5- | 1 |
| | (phenylsulfonyl)benzenesulfonamide | |
| 40 | N-{2-[4-(aminosulfonyl)phenyl]ethyl}-2-methyl-5- | 1 |
| | (phenylsulfonyl)benzenesulfonamide | |
| 41 | N-{2-[3,4-bis(benzyloxy)phenyl]ethyl}-2-methyl-5- | 1 |
| | (phenylsulfonyl)benzenesulfonamide | |
| 42 | 2-methyl-N-[2-(4-nitrophenyl)ethyl]-5- | 1 |
| | (phenylsulfonyl)benzenesulfonamide | |
| 43 | N-[2-(1,3-benzodioxol-5-yl)ethyl]-2-methyl-5- | 1 |
| | (phenylsulfonyl)benzenesulfonamide | |
| 44 | 2-methyl-5-(phenylsulfonyl)-N-{2-[3- | 1 |
| | (trifluoromethyl)phenyl]ethyl}benzenesulfonamide | |
| 45 | N-[2-(3-chlorophenyl)ethyl]-2-methyl-5- | 1 |
| | (phenylsulfonyl)benzenesulfonamide | |
| 46 | N-[2-(2,4-dichlorophenyl)ethyl]-2-methyl-5- | 1 |
| | (phenylsulfonyl)benzenesulfonamide | |
| 47 | N-[2-(2,6-dichlorophenyl)ethyl]-2-methyl-5- | 1 |
| | (phenylsulfonyl)benzenesulfonamide | |
| 48 | N-[2-(2,5-dimethoxyphenyl)ethyl]-2-methyl-5- | 1 |
| | (phenylsulfonyl)benzenesulfonamide | |
| 49 | N-[2-(3,4-dichlorophenyl)ethyl]-2-methyl-5- | 1 |
| | (phenylsulfonyl)benzenesulfonamide | |
| 50 | N-[2-(3,5-dimethoxyphenyl)ethyl]-2-methyl-5- | 1 |
| | (phenylsulfonyl)benzenesulfonamide | |
| 51 | N-[2-(4-ethoxyphenyl)ethyl]-2-methyl-5- | 1 |
| | (phenylsulfonyl)benzenesulfonamide | |
| 52 | N-[2-(3-fluorophenyl)ethyl]-2-methyl-5- | 1 |
| | (phenylsulfonyl)benzenesulfonamide | |
| 53 | N-[2-(2-fluorophenyl)ethyl]-2-methyl-5- | 1 |
| | (phenylsulfonyl)benzenesulfonamide | |
| 54 | N-[2-(3-ethoxy-4-methoxyphenyl)ethyl]-2-methyl-5- | 1 |
| | (phenylsulfonyl)benzenesulfonamide | |
| 55 | N-[2-(4-ethoxy-3-methoxyphenyl)ethyl]-2-methyl-5- | 1 |
| | (phenylsulfonyl)benzenesulfonamide | |
| 56 | N-(4-methoxybenzyl)-2-methyl-5- | 1 |
| | (phenylsulfonyl)benzenesulfonamide | |
| 57 | N-[2-(2-bromo-4,5-dimethoxyphenyl)ethyl]-2-methyl-5- | 1 |
| | (phenylsulfonyl)benzenesulfonamide | |
| 58 | N-[2-(5-bromo-2-methoxyphenyl)ethyl]-2-methyl-5- | 1 |
| | (phenylsulfonyl)benzenesulfonamide | |
| 59 | N-(2-phenylethyl)-3-(phenylsulfonyl)benzenesulfonamide | 1 |
| 60 | N-[2-(2-chlorophenyl)ethyl]-3-(phenylsulfonyl)benzenesulfonamide | 1 |
| 61 | N-[2-(2-methoxyphenyl)ethyl]-3-(phenylsulfonyl)benzenesulfonamide | 1 |
| 62 | N-[2-(3-methoxyphenyl)ethyl]-3-(phenylsulfonyl)benzenesulfonamide | 1 |
| 63 | N-[2-(3,4-dimethoxyphenyl)ethyl]-3- | 1 |
| | (phenylsulfonyl)benzenesulfonamide | |
| 64 | N-[2-(4-bromophenyl)ethyl]-3-(phenylsulfonyl)benzenesulfonamide | 1 |
| 65 | N-[2-(4-fluorophenyl)ethyl]-3-(phenylsulfonyl)benzenesulfonamide | 1 |
| 66 | N-[2-(4-chlorophenyl)ethyl]-3-(phenylsulfonyl)benzenesulfonamide | 1 |
| 67 | N-[2-(4-methoxyphenyl)ethyl]-3-(phenylsulfonyl)benzenesulfonamide | 1 |
| 68 | N-[2-(4-methylphenyl)ethyl]-3-(phenylsulfonyl)benzenesulfonamide | 1 |
| 69 | N-{2-[4-(aminosulfonyl)phenyl]ethyl}-3- | 1 |
| | (phenylsulfonyl)benzenesulfonamide | |
| 70 | N-{2-[3,4-bis(benzyloxy)phenyl]ethyl}-3- | 1 |
| | (phenylsulfonyl)benzenesulfonamide | |
| 71 | N-[2-(4-nitrophenyl)ethyl]-3-(phenylsulfonyl)benzenesulfonamide | 1 |
| 72 | N-[2-(1,3-benzodioxol-5-yl)ethyl]-3- | 1 |
| | (phenylsulfonyl)benzenesulfonamide | |
| 73 | 3-(phenylsulfonyl)-N-{2-[3- | 1 |
| | (trifluoromethyl)phenyl]ethyl}benzenesulfonamide | |
| 74 | N-[2-(3-chlorophenyl)ethyl]-3-(phenylsulfonyl)benzenesulfonamide | 1 |
| 75 | N-[2-(2,4-dichlorophenyl)ethyl]-3- | 1 |
| | (phenylsulfonyl)benzenesulfonamide | |
| 76 | N-[2-(2,6-dichlorophenyl)ethyl]-3- | 1 |
| | (phenylsulfonyl)benzenesulfonamide | |
| 77 | N-[2-(2,5-dimethoxyphenyl)ethyl]-3- | 1 |
| | (phenylsulfonyl)benzenesulfonamide | |
| 78 | N-[2-(3,4-dichlorophenyl)ethyl]-3- | 1 |
| | (phenylsulfonyl)benzenesulfonamide | |
| 79 | N-[2-(3,5-dimethoxyphenyl)ethyl]-3- | 1 |
| | (phenylsulfonyl)benzenesulfonamide | |
| 80 | N-[2-(4-ethoxyphenyl)ethyl]-3-(phenylsulfonyl)benzenesulfonamide | 1 |
| 81 | N-[2-(3-fluorophenyl)ethyl]-3-(phenylsulfonyl)benzenesulfonamide | 1 |
| 82 | N-[2-(2-fluorophenyl)ethyl]-3-(phenylsulfonyl)benzenesulfonamide | 1 |
| 83 | N-[2-(3-ethoxy-4-methoxyphenyl)ethyl]-3- | 1 |
| | (phenylsulfonyl)benzenesulfonamide | |
| 84 | N-[2-(4-ethoxy-3-methoxyphenyl)ethyl]-3- | 1 |
| | (phenylsulfonyl)benzenesulfonamide | |
| 85 | N-(4-methoxybenzyl)-3-(phenylsulfonyl)benzenesulfonamide | 1 |
| 86 | N-[2-(2-bromo-4,5-dimethoxyphenyl)ethyl]-3- | 1 |
| | (phenylsulfonyl)benzenesulfonamide | |
| 87 | N-[2-(5-bromo-2-methoxyphenyl)ethyl]-3- | 1 |
| | (phenylsulfonyl)benzenesulfonamide | |
| 88 | 5-[(4-chlorophenyl)sulfonyl]-2-methyl-N-(2- | 1 |
| | phenylethyl)benzenesulfonamide | |
| 89 | N-[2-(2-chlorophenyl)ethyl]-5-[(4-chlorophenyl)sulfonyl]-2- | 1 |
| | methylbenzenesulfonamide | |
| 90 | 5-[(4-chlorophenyl)sulfonyl]-N-[2-(2-methoxyphenyl)ethyl]-2- | 1 |
| | methylbenzenesulfonamide | |
| 91 | 5-[(4-chlorophenyl)sulfonyl]-N-[2-(3-methoxyphenyl)ethyl]-2- | 1 |
| | methylbenzenesulfonamide | |
| 92 | 5-[(4-chlorophenyl)sulfonyl]-N-[2-(3,4-dimethoxyphenyl)ethyl]-2- | 1 |
| | methylbenzenesulfonamide | |
| 93 | N-[2-(4-bromophenyl)ethyl]-5-[(4-chlorophenyl)sulfonyl]-2- | 1 |
| | methylbenzenesulfonamide | |
| 94 | 5-[(4-chlorophenyl)sulfonyl]-N-[2-(4-fluorophenyl)ethyl]-2- | 1 |
| | methylbenzenesulfonamide | |
| 95 | N-[2-(4-chlorophenyl)ethyl]-5-[(4-chlorophenyl)sulfonyl]-2- | 1 |
| | methylbenzenesulfonamide | |
| 96 | 5-[(4-chlorophenyl)sulfonyl]-N-[2-(4-methoxyphenyl)ethyl]-2- | 1 |
| | methylbenzenesulfonamide | |
| 97 | 5-[(4-chlorophenyl)sulfonyl]-2-methyl-N-[2-(4- | 1 |
| | methylphenyl)ethyl]benzenesulfonamide | |
| 98 | N-{2-[4-(aminosulfonyl)phenyl]ethyl}-5-[(4-chlorophenyl)sulfonyl]- | 1 |
| | 2-methylbenzenesulfonamide | |
| 99 | 5-[(4-chlorophenyl)sulfonyl]-2-methyl-N-[2-(4- | 1 |
| | nitrophenyl)ethyl]benzenesulfonamide | |
| 100 | N-[2-(1,3-benzodioxol-5-yl)ethyl]-5-[(4-chlorophenyl)sulfonyl]-2- | 1 |
| | methylbenzenesulfonamide | |
| 101 | 5-[(4-chlorophenyl)sulfonyl]-2-methyl-N-{2-[3- | 1 |
| | (trifluoromethyl)phenyl]ethyl}benzenesulfonamide | |
| 102 | N-[2-(3-chlorophenyl)ethyl]-5-[(4-chlorophenyl)sulfonyl]-2- | 1 |
| | methylbenzenesulfonamide | |
| 103 | 5-[(4-chlorophenyl)sulfonyl]-N-[2-(2,4-dichlorophenyl)ethyl]-2- | 1 |
| | methylbenzenesulfonamide | |
| 104 | 5-[(4-chlorophenyl)sulfonyl]-N-[2-(2,6-dichlorophenyl)ethyl]-2- | 1 |
| | methylbenzenesulfonamide | |
| 105 | 5-[(4-chlorophenyl)sulfonyl]-N-[2-(2,5-dimethoxyphenyl)ethyl]-2- | 1 |
| | methylbenzenesulfonamide | |
| 106 | 5-[(4-chlorophenyl)sulfonyl]-N-[2-(3,4-dichlorophenyl)ethyl]-2- | 1 |
| | methylbenzenesulfonamide | |
| 107 | 5-[(4-chlorophenyl)sulfonyl]-N-[2-(3,5-dimethoxyphenyl)ethyl]-2- | 1 |
| | methylbenzenesulfonamide | |
| 108 | 5-[(4-chlorophenyl)sulfonyl]-N-[2-(4-ethoxyphenyl)ethyl]-2- | 1 |
| | methylbenzenesulfonamide | |
| 109 | 5-[(4-chlorophenyl)sulfonyl]-N-[2-(3-fluorophenyl)ethyl]-2- | 1 |
| | methylbenzenesulfonamide | |
| 110 | 5-[(4-chlorophenyl)sulfonyl]-N-[2-(2-fluorophenyl)ethyl]-2- | 1 |
| | methylbenzenesulfonamide | |
| 111 | 5-[(4-chlorophenyl)sulfonyl]-N-[2-(3-ethoxy-4-methoxyphenyl)ethyl]- | 1 |
| | 2-methylbenzenesulfonamide | |
| 112 | 5-[(4-chlorophenyl)sulfonyl]-N-[2-(4-ethoxy-3-methoxyphenyl)ethyl]- | 1 |
| | 2-methylbenzenesulfonamide | |
| 113 | 5-[(4-chlorophenyl)sulfonyl]-N-(4-methoxybenzyl)-2- | 1 |
| | methylbenzenesulfonamide | |
| 114 | N-[2-(2-bromo-4,5-dimethoxyphenyl)ethyl]-5-[(4- | 1 |
| | chlorophenyl)sulfonyl]-2-methylbenzenesulfonamide | |
| 115 | N-[2-(5-bromo-2-methoxyphenyl)ethyl]-5-[(4-chlorophenyl)sulfonyl]- | 1 |
| | 2-methylbenzenesulfonamide | |
| 116 | 2-methyl-5-[(4-methylphenyl)sulfonyl]-N-(2- | 1 |
| | phenylethyl)benzenesulfonamide | |
| 117 | N-[2-(2-chlorophenyl)ethyl]-2-methyl-5-[(4- | 1 |
| | methylphenyl)sulfonyl]benzenesulfonamide | |
| 118 | N-[2-(2-methoxyphenyl)ethyl]-2-methyl-5-[(4- | 1 |
| | methylphenyl)sulfonyl]benzenesulfonamide | |
| 119 | N-[2-(3-methoxyphenyl)ethyl]-2-methyl-5-[(4- | 1 |
| | methylphenyl)sulfonyl]benzenesulfonamide | |
| 120 | N-[2-(3,4-dimethoxyphenyl)ethyl]-2-methyl-5-[(4- | 1 |
| | methylphenyl)sulfonyl]benzenesulfonamide | |
| 121 | N-[2-(4-bromophenyl)ethyl]-2-methyl-5-[(4- | 1 |
| | methylphenyl)sulfonyl]benzenesulfonamide | |
| 122 | N-[2-(4-fluorophenyl)ethyl]-2-methyl-5-[(4- | 1 |
| | methylphenyl)sulfonyl]benzenesulfonamide | |
| 123 | N-[2-(4-chlorophenyl)ethyl]-2-methyl-5-[(4- | 1 |
| | methylphenyl)sulfonyl]benzenesulfonamide | |
| 124 | N-[2-(4-methoxyphenyl)ethyl]-2-methyl-5-[(4- | 1 |
| | methylphenyl)sulfonyl]benzenesulfonamide | |
| 125 | 2-methyl-N-[2-(4-methylphenyl)ethyl]-5-[(4- | 1 |
| | methylphenyl)sulfonyl]benzenesulfonamide | |
| 126 | N-{2-[4-(aminosulfonyl)phenyl]ethyl}-2-methyl-5-[(4- | 1 |
| | methylphenyl)sulfonyl]benzenesulfonamide | |
| 127 | 2-methyl-5-[(4-methylphenyl)sulfonyl]-N-[2-(4- | 1 |
| | nitrophenyl)ethyl]benzenesulfonamide | |
| 128 | N-[2-(1,3-benzodioxol-5-yl)ethyl]-2-methyl-5-[(4- | 1 |
| | methylphenyl)sulfonyl]benzenesulfonamide | |
| 129 | 2-methyl-5-[(4-methylphenyl)sulfonyl]-N-{2-[3- | 1 |
| | (trifluoromethyl)phenyl]ethyl}benzenesulfonamide | |
| 11 | N-[2-(3-chlorophenyl)ethyl]-2-methyl-5-[(4- | 1 |
| | methylphenyl)sulfonyl]benzenesulfonamide | |
| 131 | N-[2-(2,4-dichlorophenyl)ethyl]-2-methyl-5-[(4- | 1 |
| | methylphenyl)sulfonyl]benzenesulfonamide | |
| 132 | N-[2-(2,6-dichlorophenyl)ethyl]-2-methyl-5-[(4- | 1 |
| | methylphenyl)sulfonyl]benzenesulfonamide | |
| 133 | N-[2-(2,5-dimethoxyphenyl)ethyl]-2-methyl-5-[(4- | 1 |
| | methylphenyl)sulfonyl]benzenesulfonamide | |
| 134 | N-[2-(3,4-dichlorophenyl)ethyl]-2-methyl-5-[(4- | 1 |
| | methylphenyl)sulfonyl]benzenesulfonamide | |
| 135 | N-[2-(3,5-dimethoxyphenyl)ethyl]-2-methyl-5-[(4- | 1 |
| | methylphenyl)sulfonyl]benzenesulfonamide | |
| 136 | N-[2-(4-ethoxyphenyl)ethyl]-2-methyl-5-[(4- | 1 |
| | methylphenyl)sulfonyl]benzenesulfonamide | |
| 137 | N-[2-(3-fluorophenyl)ethyl]-2-methyl-5-[(4- | 1 |
| | methylphenyl)sulfonyl]benzenesulfonamide | |
| 138 | N-[2-(2-fluorophenyl)ethyl]-2-methyl-5-[(4- | 1 |
| | methylphenyl)sulfonyl]benzenesulfonamide | |
| 139 | N-[2-(2-bromo-4,5-dimethoxyphenyl)ethyl]-2-methyl-5-[(4- | 1 |
| | methylphenyl)sulfonyl]benzenesulfonamide | |
| 140 | N-[2-(5-bromo-2-methoxyphenyl)ethyl]-2-methyl-5-[(4- | 1 |
| | methylphenyl)sulfonyl]benzenesulfonamide | |
| 141 | 2-ethyl-N-(2-phenylethyl)-5-(phenylsulfonyl)benzenesulfonamide | 1 |
| 142 | 2-ethyl-N-[2-(2-methoxyphenyl)ethyl]-5- | 1 |
| | (phenylsulfonyl)benzenesulfonamide | |
| 143 | 2-ethyl-N-[2-(3-methoxyphenyl)ethyl]-5- | 1 |
| | (phenylsulfonyl)benzenesulfonamide | |
| 144 | N-[2-(3,4-dimethoxyphenyl)ethyl]-2-ethyl-5- | 1 |
| | (phenylsulfonyl)benzenesulfonamide | |
| 145 | N-{2-[4-(aminosulfonyl)phenyl]ethyl}-2-ethyl-5- | 1 |
| | (phenylsulfonyl)benzenesulfonamide | |
| 146 | 2-ethyl-N-[2-(3-fluorophenyl)ethyl]-5- | 1 |
| | (phenylsulfonyl)benzenesulfonamide | |
| 147 | 2-ethyl-N-[2-(2-fluorophenyl)ethyl]-5- | 1 |
| | (phenylsulfonyl)benzenesulfonamide | |
| 148 | N-[2-(3-ethoxy-4-methoxyphenyl)ethyl]-2-ethyl-5- | 1 |
| | (phenylsulfonyl)benzenesulfonamide | |
| 149 | N-[2-(4-ethoxy-3-methoxyphenyl)ethyl]-2-ethyl-5- | 1 |
| | (phenylsulfonyl)benzenesulfonamide | |
| 150 | 2-methoxy-N-(2-phenylethyl)-5-(phenylsulfonyl)benzenesulfonamide | 1 |
| 151 | 2-methoxy-N-[2-(2-methoxyphenyl)ethyl]-5- | 1 |
| | (phenylsulfonyl)benzenesulfonamide | |
| 152 | 2-methoxy-N-[2-(3-methoxyphenyl)ethyl]-5- | 1 |
| | (phenylsulfonyl)benzenesulfonamide | |
| 153 | N-[2-(3,4-dimethoxyphenyl)ethyl]-2-methoxy-5- | 1 |
| | (phenylsulfonyl)benzenesulfonamide | |
| 154 | N-{2-[4-(aminosulfonyl)phenyl]ethyl}-2-methoxy-5- | 1 |
| | (phenylsulfonyl)benzenesulfonamide | |
| 155 | N-[2-(3-fluorophenyl)ethyl]-2-methoxy-5- | 1 |
| | (phenylsulfonyl)benzenesulfonamide | |
| 156 | N-[2-(2-fluorophenyl)ethyl]-2-methoxy-5- | 1 |
| | (phenylsulfonyl)benzenesulfonamide | |
| 157 | N-[2-(3-ethoxy-4-methoxyphenyl)ethyl]-2-methoxy-5- | 1 |
| | (phenylsulfonyl)benzenesulfonamide | |
| 158 | N-[2-(4-ethoxy-3-methoxyphenyl)ethyl]-2-methoxy-5- | 1 |
| | (phenylsulfonyl)benzenesulfonamide | |
| 159 | N³-({5-[(4-fluorophenyl)sulfonyl]-2-methylphenyl}sulfonyl)-beta- | 1 |
| | alaninamide | |
| 160 | methyl N-({5-[(4-fluorophenyl)sulfonyl]-2-methylphenyl}sulfonyl)- | 1 |
| | beta-alaninate | |
| 161 | N-(2-cyanoethyl)-5-[(4-fluorophenyl)sulfonyl]-2- | 1 |
| | methylbenzenesulfonamide | |
| 162 | 5-[(4-fluorophenyl)sulfonyl]-2-methyl-N-(2-pyridin-2- | 1 |
| | ylethyl)benzenesulfonamide | |
| 163 | 5-[(4-fluorophenyl)sulfonyl]-2-methyl-N-(3-morpholin-4- | 1 |
| | ylpropyl)benzenesulfonamide | |
| 164 | 5-[(4-fluorophenyl)sulfonyl]-2-methyl-N-[2-(1-methylpyrrolidin-2- | 1 |
| | yl)ethyl]benzenesulfonamide | |
| 165 | 5-[(4-fluorophenyl)sulfonyl]-2-methyl-N-(2-pyridin-4- | 1 |
| | ylethyl)benzenesulfonamide | |
| 166 | 5-[(4-fluorophenyl)sulfonyl]-2-methyl-N-(2-piperidin-1- | 1 |
| | ylethyl)benzenesulfonamide | |
| 167 | 5-[(4-fluorophenyl)sulfonyl]-N-[2-(1H-imidazol-4-yl)ethyl]-2- | 1 |
| | methylbenzenesulfonamide | |
| 168 | N-{2-[({5-[(4-fluorophenyl)sulfonyl]-2- | 1 |
| | methylphenyl}sulfonyl)amino]ethyl}acetamide | |
| 169 | 5-[(4-fluorophenyl)sulfonyl]-2-methyl-N-(2-morpholin-4- | 1 |
| | ylethyl)benzenesulfonamide | |
| 170 | 5-[(4-fluorophenyl)sulfonyl]-2-methyl-N-[3-(2-oxopyrrolidin-1- | 1 |
| | yl)propyl]benzenesulfonamide | |
| 171 | N-[2-(diethylamino)ethyl]-5-[(4-fluorophenyl)sulfonyl]-2- | 1 |
| | methylbenzenesulfonamide | |
| 172 | N-[2-(dimethylamino)ethyl]-5-[(4-fluorophenyl)sulfonyl]-2- | 1 |
| | methylbenzenesulfonamide | |
| 173 | 5-[(4-fluorophenyl)sulfonyl]-N-(3-methoxypropyl)-2- | 1 |
| | methylbenzenesulfonamide | |
| 174 | N-[3-(dimethylamino)propyl]-5-[(4-fluorophenyl)sulfonyl]-2- | 1 |
| | methylbenzenesulfonamide | |
| 175 | 5-[(4-fluorophenyl)sulfonyl]-N-(2-methoxyethyl)-2- | 1 |
| | methylbenzenesulfonamide | |
| 176 | N-[3-(diethylamino)propyl]-5-[(4-fluorophenyl)sulfonyl]-2- | 1 |
| | methylbenzenesulfonamide | |
| 177 | 5-[(4-fluorophenyl)sulfonyl]-N-[3-(1H-imidazol-1-yl)propyl]-2- | 1 |
| | methylbenzenesulfonamide | |
| 178 | 5-[(4-fluorophenyl)sulfonyl]-2-methyl-N-(3-pyrrolidin-1- | 1 |
| | ylpropyl)benzenesulfonamide | |
| 179 | 5-[(4-fluorophenyl)sulfonyl]-2-methyl-N-[3-(4-methylpiperazin-1- | 1 |
| | yl)propyl]benzenesulfonamide | |

### EXAMPLE 180

### N-({5-[(4-fluorophenyl)sulfonyl]-2-methylphenyl}sulfonyl)-beta-alanine

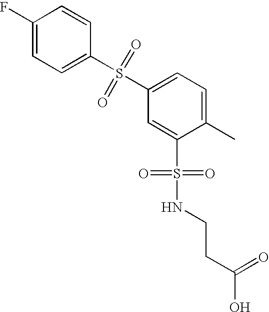

To methyl N-({5-[(4-fluorophenyl)sulfonyl]-2-methylphenyl}sulfonyl)-beta-alaninate (Example 160) (30 mg, 0.07 mmol) in methanol (1 mL) was added 2.0 M of lithium hydroxide aqueous solution (1 mL, 2 mmol). The reaction mixture was stirred at room temperature for 5 hours and purified by reverse phase column to yield N-({5-[(4-fluoro-phenyl) sulfonyl]-2-methylphenyl}sulfonyl)-beta-alanine (25 mg, 86% yield).

HPLC (Method 1): Rt=2.56 min; MS 402.04 [M+H]

### EXAMPLE 181-202

The compounds of Examples 181 to 201 were prepared generally according to the procedures described in Example 1.

| | | Prepared |
|---|---|---|
| | | according |
| | | description |
| Example | | provided in |
| No. | Compound Name | Example No. |
| 181 | N³-{[3-(phenylsulfonyl)phenyl]sulfonyl}-beta-alaninamide | 1 |
| 182 | methyl N-{[3-(phenylsulfonyl)phenyl]sulfonyl}-beta-alaninate | 1 |
| 183 | N-(2-cyanoethyl)-3-(phenylsulfonyl)benzenesulfonamide | 1 |
| 184 | 3-(phenylsulfonyl)-N-(2-pyridin-2-ylethyl)benzenesulfonamide | 1 |
| 185 | N-(3-morpholin-4-ylpropyl)-3-(phenylsulfonyl)benzenesulfonamide | 1 |
| 186 | N-[2-(1-methylpyrrolidin-2-yl)ethyl]-3- | 1 |
| | (phenylsulfonyl)benzenesulfonamide | |
| 187 | 3-(phenylsulfonyl)-N-(2-pyridin-4-ylethyl)benzenesulfonamide | 1 |
| 188 | 3-(phenylsulfonyl)-N-(2-piperidin-1-ylethyl)benzenesulfonamide | 1 |
| 189 | N-[2-(1H-imidazol-4-yl)ethyl]-3-(phenylsulfonyl)benzenesulfonamide | 1 |
| 190 | N-[2-({[3-(phenylsulfonyl)phenyl]sulfonyl}amino)ethyl]acetamide | 1 |
| 191 | N-(2-morpholin-4-ylethyl)-3-(phenylsulfonyl)benzenesulfonamide | 1 |
| 192 | N-[3-(2-oxopyrrolidin-1-yl)propyl]-3- | 1 |
| | (phenylsulfonyl)benzenesulfonamide | |
| 193 | N-[2-(diethylamino)ethyl]-3-(phenylsulfonyl)benzenesulfonamide | 1 |
| 194 | N-[2-(dimethylamino)ethyl]-3-(phenylsulfonyl)benzenesulfonamide | 1 |
| 195 | N-(3-methoxypropyl)-3-(phenylsulfonyl)benzenesulfonamide | 1 |
| 196 | N-[3-(dimethylamino)propyl]-3-(phenylsulfonyl)benzenesulfonamide | 1 |
| 197 | N-(2-methoxyethyl)-3-(phenylsulfonyl)benzenesulfonamide | 1 |
| 198 | N-[3-(diethylamino)propyl]-3-(phenylsulfonyl)benzenesulfonamide | 1 |
| 199 | N-[3-(1H-imidazol-1-yl)propyl]-3- | 1 |
| | (phenylsulfonyl)benzenesulfonamide | |
| 200 | 3-(phenylsulfonyl)-N-(3-pyrrolidin-1-ylpropyl)benzenesulfonamide | 1 |
| 201 | N-[3-(4-methylpiperazin-1-yl)propyl]-3- | 1 |
| | (phenylsulfonyl)benzenesulfonamide | |

### EXAMPLE 202

### N-{[3-(phenylsulfonyl)phenyl]sulfonyl)-beta-alanine

The compound of Example 202 was prepared generally according to the procedures described in Example 180.

### EXAMPLES 203-221

The compounds of Examples 203 to 221 were prepared generally according to the procedures described in Example 1.

| | | Prepared |
|---|---|---|
| | | according |
| | | description |
| Example | | provided in |
| No. | Compound Name | Example No. |
| 203 | N³-{[2-methyl-5- | 1 |
| | (phenylsulfonyl)phenyl]sulfonyl}-beta- | |
| | alaninamide | |
| 204 | methyl N-{[2-methyl-5- | 1 |
| | (phenylsulfonyl)phenyl]sulfonyl}-beta- | |
| | alaninate | |
| 205 | 2-methyl-5-(phenylsulfonyl)-N-(2-pyridin-2- | 1 |
| | ylethyl)benzenesulfonamide | |
| 206 | 2-methyl-N-(3-morpholin-4-ylpropyl)- | 1 |
| | 5-(phenylsulfonyl)benzenesulfonamide | |
| 207 | 2-methyl-N-[2-(1-methylpyrrolidin-2-yl)ethyl]- | 1 |
| | 5-(phenylsulfonyl)benzenesulfonamide | |
| 208 | 2-methyl-5-(phenylsulfonyl)-N-(2-pyridin-4- | 1 |
| | ylethyl)benzenesulfonamide | |
| 209 | 2-methyl-5-(phenylsulfonyl)-N-(2-piperidin-1- | 1 |
| | ylethyl)benzenesulfonamide | |
| 210 | N-[2-(1H-imidazol-4-yl)ethyl]-2-methyl-5- | 1 |
| | (phenylsulfonyl)benzenesulfonamide | |
| 211 | 2-methyl-N-(2-morpholin-4-ylethyl)-5- | 1 |
| | (phenylsulfonyl)benzenesulfonamide | |
| 212 | 2-methyl-N-[3-(2-oxopyrrolidin-1-yl)propyl]-5- | 1 |
| | (phenylsulfonyl)benzenesulfonamide | |
| 213 | N-[2-(diethylamino)ethyl]-2-methyl-5- | 1 |
| | (phenylsulfonyl)benzenesulfonamide | |
| 214 | N-[2-(dimethylamino)ethyl]-2-methyl-5- | 1 |
| | (phenylsulfonyl)benzenesulfonamide | |
| 215 | N-(3-methoxypropyl)-2-methyl-5- | 1 |
| | (phenylsulfonyl)benzenesulfonamide | |
| 216 | N-[3-(dimethylamino)propyl]-2-methyl-5- | 1 |
| | (phenylsulfonyl)benzenesulfonamide | |
| 217 | N-(2-methoxyethyl)-2-methyl-5- | 1 |
| | (phenylsulfonyl)benzenesulfonamide | |
| 218 | N-[3-(diethylamino)propyl]-2-methyl-5- | 1 |
| | (phenylsulfonyl)benzenesulfonamide | |
| 219 | N-[3-(1H-imidazol-1-yl)propyl]-2-methyl-5- | 1 |
| | (phenylsulfonyl)benzenesulfonamide | |
| 220 | 2-methyl-5-(phenylsulfonyl)-N-(3-pyrrolidin-1- | 1 |
| | ylpropyl)benzenesulfonamide | |
| 221 | 2-methyl-N-[3-(4-methylpiperazin-1-yl)propyl]- | 1 |
| | 5-(phenylsulfonyl)benzenesulfonamide | |

### EXAMPLES 222, 223, AND 225 TO 229

The compounds of Examples 222, 223, and 225 to 229 were prepared generally according to the procedures described in Example 230.

| | | Prepared |
|---|---|---|
| | | according |
| | | description |
| Example | | provided in |
| No. | Compound Name | Example No. |
| 222 | methyl N-{[2-ethyl-5- | 230 |
| | (phenylsulfonyl)phenyl]sulfonyl}-beta- | |
| | alaninate | |
| 223 | 2-ethyl-5-(phenylsulfonyl)-N-(2-pyridin-2- | 230 |
| | ylethyl)benzenesulfonamide | |
| 225 | 2-ethyl-N-[2-(1H-imidazol-4-yl)ethyl]-5- | 230 |
| | (phenylsulfonyl)benzenesulfonamide | |
| 226 | 2-ethyl-N-(2-morpholin-4-ylethyl)-5- | 230 |
| | (phenylsulfonyl)benzenesulfonamide | |
| 227 | 2-ethyl-N-(3-methoxypropyl)-5- | 230 |
| | (phenylsulfonyl)benzenesulfonamide | |
| 228 | N-[3-(dimethylamino)propyl]-2-ethyl-5- | 230 |
| | (phenylsulfonyl)benzenesulfonamide | |
| 229 | 2-ethyl-N-(2-methoxyethyl)-5- | 230 |
| | (phenylsulfonyl)benzenesulfonamide | |

### EXAMPLE 230

### N-[3-(diethylamino) propyl]-2-ethyl-5-(phenylsulfonyl)benzene sulfonamide

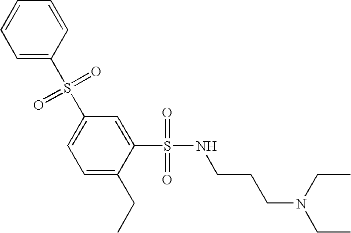

Step 1: To 4-Ethyl-benzenesulfonyl chloride (4.12 g, 20.0 mmol) and aluminum chloride (3.20 g, 24.0 mmol) was added benzene (10 mL). The reaction mixture was stirred at room temperature over night and then poured into water. The resulting solution was diluted with ethyl acetate (200 mL) and the organic layer was washed with 1M aqueous sodium hydroxide solution (100 mL) and brine (100 mL). The organic layer was dried over sodium sulfate, filtered and concentrated to give 1-Benzenesulfonyl-4-ethyl-benzene (4.58 g, 92.1% yield) as a white solid.

¹HNMR (CDCl₃) δ 8.09 (2H, dd), 7.89 (2H, dd), 7.94 (2H, dd), 7.51 (m, 2H), 7.29 (1H, m), 7.00 (2H, dd), 2.86 (2H, qt), 1.22 (3H, t).

Step 2: Following the same procedure described in Example 1 (step 1), 5-Benzenesulfonyl-2-ethyl-benzenesulfonyl chloride was made quanitively.

Step 3: Following the same procedure described in Example 1 (step 2), a 0.1 mmol scale reaction was set up and N-[3-(diethylamino) propyl]-2-ethyl-5-(phenylsulfonyl)benzene sulfonamide (38 mg, 86% yield) was synthesized.

HPLC (Method 2): Rt=2.8 min; MS 439.2 [M+H]

### EXAMPLES 231-243

The compounds of Examples 231 to 243 were prepared generally according to the procedures described in Example 230.

| | | Prepared |
|---|---|---|
| | | according |
| | | description |
| Example | | provided in |
| No. | Compound Name | Example No. |
| 231 | 2-ethyl-N-[3-(1H-imidazol-1-yl)propyl]-5- | 230 |
| | (phenylsulfonyl)benzenesulfonamide | |
| 232 | 2-ethyl-5-(phenylsulfonyl)-N-(3-pyrrolidin-1- | 230 |
| | ylpropyl)benzenesulfonamide | |
| 233 | methyl N-{[2-methoxy-5- | 230 |
| | (phenylsulfonyl)phenyl]sulfonyl}-beta- | |
| | alaninate | |
| 234 | 2-methoxy-5-(phenylsulfonyl)-N-(2-pyridin-2- | 230 |
| | ylethyl)benzenesulfonamide | |
| 235 | 2-methoxy-5-(phenylsulfonyl)-N-(2-pyridin-4- | 230 |
| | ylethyl)benzenesulfonamide | |
| 236 | N-[2-(1H-imidazol-4-yl)ethyl]-2-methoxy-5- | 230 |
| | (phenylsulfonyl)benzenesulfonamide | |
| 237 | 2-methoxy-N-(2-morpholin-4-ylethyl)-5- | 230 |
| | (phenylsulfonyl)benzenesulfonamide | |
| 238 | 2-methoxy-N-(3-methoxypropyl)-5- | 230 |
| | (phenylsulfonyl)benzenesulfonamide | |
| 239 | N-[3-(dimethylamino)propyl]-2-methoxy-5- | 230 |
| | (phenylsulfonyl)benzenesulfonamide | |
| 240 | 2-methoxy-N-(2-methoxyethyl)-5- | 230 |
| | (phenylsulfonyl)benzenesulfonamide | |
| 241 | N-[3-(diethylamino)propyl]-2-methoxy-5- | 230 |
| | (phenylsulfonyl)benzenesulfonamide | |
| 242 | N-[3-(1H-imidazol-1-yl)propyl]-2-methoxy-5- | 230 |
| | (phenylsulfonyl)benzenesulfonamide | |
| 243 | 2-methoxy-5-(phenylsulfonyl)-N-(3-pyrrolidin- | 230 |
| | 1-ylpropyl)benzenesulfonamide | |

### EXAMPLES 244-265

The compounds of Examples 244 to 265 were prepared generally according to the procedures described in Example 1.

| | | Prepared |
|---|---|---|
| | | according |
| | | description |
| Example | | provided in |
| No. | Compound Name | Example No. |
| 244 | 2-methyl-N-(3-phenylpropyl)-5-(phenylsulfonyl)benzenesulfonamide | 1 |
| 245 | 2-ethyl-N-(3-phenylpropyl)-5-(phenylsulfonyl)benzenesulfonamide | 1 |
| 246 | N-2,3-dihydro-1H-inden-2-yl-2-methyl-5- | 1 |
| | (phenylsulfonyl)benzenesulfonamide | |
| 247 | 2-methyl-N-[(1S,2R)-2-phenylcyclopropyl]-5- | 1 |
| | (phenylsulfonyl)benzenesulfonamide | |
| 248 | 2-methyl-N-[(2R)-2-phenylpropyl]-5- | 1 |
| | (phenylsulfonyl)benzenesulfonamide | |
| 249 | N-2,3-dihydro-1H-inden-2-yl-5-[(4-fluorophenyl)sulfonyl]-2- | 1 |
| | methylbenzenesulfonamide | |
| 250 | 5-[(4-fluorophenyl)sulfonyl]-2-methyl-N-[(1S,2R)-2- | 1 |
| | phenylcyclopropyl]benzenesulfonamide | |
| 251 | 5-[(4-fluorophenyl)sulfonyl]-2-methyl-N-[(2R)-2- | 1 |
| | phenylpropyl]benzenesulfonamide | |
| 252 | N-2,3-dihydro-1H-inden-2-yl-2-methyl-5-[(4- | 1 |
| | methylphenyl)sulfonyl]benzenesulfonamide | |
| 253 | 2-methyl-5-[(4-methylphenyl)sulfonyl]-N-[(1S,2R)-2- | 1 |
| | phenylcyclopropyl]benzenesulfonamide | |
| 254 | 2-methyl-5-[(4-methylphenyl)sulfonyl]-N-[(2R)-2- | 1 |
| | phenylpropyl]benzenesulfonamide | |
| 255 | 5-[(4-chlorophenyl)sulfonyl]-N-2,3-dihydro-1H-inden-2-yl-2- | 1 |
| | methylbenzenesulfonamide | |
| 256 | 5-[(4-chlorophenyl)sulfonyl]-2-methyl-N-[(1S,2R)-2- | 1 |
| | phenylcyclopropyl]benzenesulfonamide | |
| 257 | 5-[(4-chlorophenyl)sulfonyl]-2-methyl-N-[(2R)-2- | 1 |
| | phenylpropyl]benzenesulfonamide | |
| 258 | N-2,3-dihydro-1H-inden-2-yl-3-(phenylsulfonyl)benzenesulfonamide | 1 |
| 259 | N-[(1S,2R)-2-phenylcyclopropyl]-3- | 1 |
| | (phenylsulfonyl)benzenesulfonamide | |
| 260 | N-[(2R)-2-phenylpropyl]-3-(phenylsulfonyl)benzenesulfonamide | 1 |
| 261 | 5-[(4-fluorophenyl)sulfonyl]-2-methyl-N-[(2S)-2- | 1 |
| | phenylpropyl]benzenesulfonamide | |
| 262 | 2-methyl-N-[(2S)-2-phenylpropyl]-5- | 1 |
| | (phenylsulfonyl)benzenesulfonamide | |
| 263 | N-[(2S)-2-phenylpropyl]-3-(phenylsulfonyl)benzenesulfonamide | 1 |
| 264 | 2-methyl-5-[(4-methylphenyl)sulfonyl]-N-[(2S)-2- | 1 |
| | phenylpropyl]benzenesulfonamide | |
| 265 | 5-[(4-chlorophenyl)sulfonyl]-2-methyl-N-[(2S)-2- | 1 |
| | phenylpropyl]benzenesulfonamide | |

### EXAMPLES 266-271

The compounds of Examples 266 to 271 were prepared generally according to the procedures described in Example 230.

| | | Prepared |
|---|---|---|
| | | according |
| | | description |
| Example | | provided in |
| No. | Compound Name | Example No. |
| 266 | N-2,3-dihydro-1H-inden-2-yl-2-ethyl-5- | 230 |
| | (phenylsulfonyl)benzenesulfonamide | |
| 267 | 2-ethyl-N-[(1S,2R)-2-phenylcyclopropyl]-5- | 230 |
| | (phenylsulfonyl)benzenesulfonamide | |
| 268 | 2-ethyl-N-[(2R)-2-phenylpropyl]-5- | 230 |
| | (phenylsulfonyl)benzenesulfonamide | |
| 269 | N-2,3-dihydro-1H-inden-2-yl-2-methoxy-5- | 230 |
| | (phenylsulfonyl)benzenesulfonamide | |
| 270 | 2-methoxy-N-[(1S,2R)-2-phenylcyclopropyl]-5- | 230 |
| | (phenylsulfonyl)benzenesulfonamide | |
| 271 | 2-methoxy-N-[(2R)-2-phenylpropyl]-5- | 230 |
| | (phenylsulfonyl)benzenesulfonamide | |

### EXAMPLES 272-275

The compounds of Examples 272 to 275 were prepared generally according to the procedures described in Example 276.

| | | Prepared |
|---|---|---|
| | | according |
| Exam- | | description |
| ple | | provided in |
| No. | Compound Name | Example No. |
| 272 | N-[2-(3,4-dimethoxyphenyl)ethyl]-N,2-dimethyl- | 276 |
| | 5-(phenylsulfonyl)benzenesulfonamide | |
| 273 | N-allyl-N-[2-(3,4-dimethoxyphenyl)ethyl]-2- | 276 |
| | methyl-5-(phenylsulfonyl)benzenesulfonamide | |
| 274 | N-[2-(3,4-dimethoxyphenyl)ethyl]-2-methyl-5- | 276 |
| | (phenylsulfonyl)-N-prop-2- | |
| | ynylbenzenesulfonamide | |
| 275 | N-[2-(2-fluorophenyl)ethyl]-N,2-dimethyl-5-[(4- | 276 |
| | methylphenyl)sulfonyl]benzenesulfonamide | |

### EXAMPLE 276

### N-allyl-N-[2-(2-fluorophenyl)ethyl]-2-methyl-5-[(4-methylphenyl)sulfonyl]benzene sulfonamide

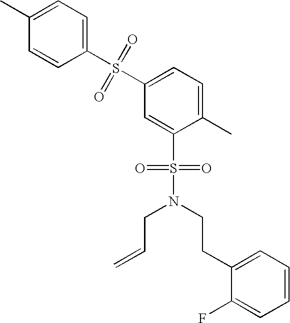

To N-[2-(2-fluorophenyl) ethyl]-2-methyl-5-[(4-methylphenyl) sulfonyl]benzene sulfonamide (Example 138) (22 mg, 0.05 mmol) in DMF (1 mL) was added sodium hydride (4 mg, 0.1 mmol). The reaction mixture was stirred at room temperature for 20 minutes, and then allyl bromide (12 mg, 0.1 mmol) was added. The reaction mixture was stirred at room temperature for another 2 hours and purified by a reverse phase column to yield N-allyl-N-[2-(2-fluorophenyl)ethyl]-2-methyl-5-[(4-methylphenyl)sulfonyl]benzene sulfonamide (8.2 mg, 34% yield).

### EXAMPLES 277

### N-[2-(2-fluorophenyl)ethyl]-2-methyl-5-[(4-methylphenyl)sulfonyl]-N-prop-2-ynylbenzenesulfonamide

The compound of Example 277 was prepared generally according to the procedures described in Example 276.

### EXAMPLES 278-283

The compounds of Examples 278 to 283 were prepared generally according to the procedures described in Example 1.

| | | Prepared |
|---|---|---|
| | | according |
| Exam- | | description |
| ple | | provided in |
| No. | Compound Name | Example No. |
| 278 | N,2-dimethyl-N-(2-phenylethyl)-5- | 1 |
| | (phenylsulfonyl)benzenesulfonamide | |
| 279 | 1-{[2-methyl-5-(phenylsulfonyl)phenyl]sulfonyl}- | 1 |
| | 4-(2-oxo-2-pyrrolidin-1-ylethyl)piperazine | |
| 280 | N,N-diethyl-N-[2-(4-{[2-methyl-5- | 1 |
| | (phenylsulfonyl)phenyl]sulfonyl}piperazin-1- | |
| | yl)ethyl]amine | |
| 281 | 4-[2-(4-{[2-methyl-5- | 1 |
| | (phenylsulfonyl)phenyl]sulfonyl}piperazin-1- | |
| | yl)ethyl]morpholine | |
| 282 | 1-{[2-methyl-5-(phenylsulfonyl)phenyl]sulfonyl}- | 1 |
| | 4-(2-pyrrolidin-1-ylethyl)piperazine | |
| 283 | 4-[3-(4-{[2-methyl-5- | 1 |
| | (phenylsulfonyl)phenyl]sulfonyl}piperazin-1- | |
| | yl)propyl]morpholine | |

### EXAMPLES 284-289

The compounds of Examples 284 to 289 were prepared generally according to the procedures described in Example 230.

| | | Prepared |
|---|---|---|
| | | according |
| Exam- | | description |
| ple | | provided in |
| No. | Compound Name | Example No. |
| 284 | 2-ethyl-N-methyl-N-(2-phenylethyl)-5- | 230 |
| | (phenylsulfonyl)benzenesulfonamide | |
| 285 | 1-{[2-ethyl-5-(phenylsulfonyl)phenyl]sulfonyl}-4- | 230 |
| | (2-oxo-2-pyrrolidin-1-ylethyl)piperazine | |
| 286 | N,N-diethyl-N-[2-(4-{[2-ethyl-5- | 230 |
| | (phenylsulfonyl)phenyl]sulfonyl}piperazin-1- | |
| | yl)ethyl]amine | |
| 287 | 4-[2-(4-{[2-ethyl-5- | 230 |
| | (phenylsulfonyl)phenyl]sulfonyl}piperazin-1- | |
| | yl)ethyl]morpholine | |
| 288 | 1-{[2-ethyl-5-(phenylsulfonyl)phenyl]sulfonyl}- | 230 |
| | 4-(2-pyrrolidin-1-ylethyl)piperazine | |
| 289 | 4-[3-(4-{[2-ethyl-5- | 230 |
| | (phenylsulfonyl)phenyl]sulfonyl}piperazin-1- | |
| | yl)propyl]morpholine | |

The following table provides the HPLC retention time and mass spec data for the compounds of Examples 1 to 289.

| | Ret.Time | |
|---|---|---|
| Example | (minutes) | MS |
| 1 | 3.1 | 416 [M + H |
| 2 | 2.2 | 889 [2M + Na] |
| 3 | 2.34 | 468 [M + H] |
| 4 | 2.32 | 464 [M + H] |
| 5 | 2.29 | 464 [M + H] |
| 6 | 2.2 | 494 [M + H] |
| 7 | 2.4 | 512 [M + H] |
| 8 | 2.32 | 469 [M + NH4] |
| 9 | 2.3 | 466 [M − H] |
| 10 | 2.25 | 464 [M + H] |
| 11 | 2.35 | 448 [M + H] |
| 12 | 1.96 | 513 [M + H] |
| 13 | 2.6 | 64 [M − H] |
| 14 | 2.2 | 496 [M + NH4] |
| 15 | 2.4 | 496 [M + NH4] |
| 16 | 2.4 | 519 [M + NH4] |
| 17 | 2.4 | 486 [M + NH4] |
| 18 | 2.4 | 519 [M + NH4] |
| 19 | 2.2 | 502 [M + H] |
| 20 | 2.3 | 494 [M + H] |
| 21 | 2.4 | 500 [M − H] |
| 22 | 3.1 | 494 [M + H] |
| 23 | 3.2 | 478 [M + H] |
| 24 | 2.3 | 452 [M + H] |
| 25 | 3.1 | 469 [M + NH4] |
| 26 | 2.2 | 525 [M + NH4] |
| 27 | 2.2 | 525 [M + NH4] |
| 28 | 2.2 | 448 [M − H] |
| 29 | 2.31 | 572 [M − H] |
| 30 | 2.4 | 540 [M − H] |
| 31 | 2.19 | 923 [2M + Na] |
| 32 | 3.1 | 446 [M + H] |
| 33 | 3.1 | 446 [M + H] |
| 34 | 2.96 | 498 [M + Na] |
| 35 | 3.2 | 516 [M + Na] |
| 36 | 3.12 | 434 [M + H] |
| 37 | 3.25 | 450 [M + H] |
| 38 | 3.08 | 446 [M + H] |
| 39 | 3.2 | 881 [2M + Na] |
| 40 | 1.87 | 493 [M − H] |
| 41 | 3.5 | 628 [M + H] |
| 42 | 3.0 | 483 [M + Na] |
| 43 | 3.0 | 482 [M + Na] |
| 44 | 3.25 | 506 [M + Na] |
| 45 | 3.22 | 451 [M + H] |
| 46 | 3.22 | 484 [M + H] |
| 47 | 3.27 | 484 [M + H] |
| 48 | 3.12 | 476 [M + H] |
| 49 | 3.32 | 484 [M + H] |
| 50 | 3.09 | 476 [M + H] |
| 51 | 3.18 | 460 [M + H] |
| 52 | 3.12 | 434 [M + H] |
| 53 | 3.12 | 434 [M + H] |
| 54 | 3.06 | 490 [M + H] |
| 55 | 3.07 | 490 [M + H] |
| 56 | 2.08 | 430 [M − H] |
| 57 | 3.1 | 555 [M + H] |
| 58 | 3.29 | 525 [M + H] |
| 59 | 3.08 | 424 [M + Na] |
| 60 | 3.16 | 458 [M + Na] |
| 61 | 3.11 | 885 [2M + Na]] |
| 62 | 2.19 | 430 [M − H] |
| 63 | 2.07 | 460 [M − H] |
| 64 | 3.22 | 502 [M + Na] |
| 65 | 3.08 | 442 [M + Na] |
| 66 | 3.19 | 458 [M + Na] |
| 67 | 3.04 | 432 [M + H] |
| 68 | 3.18 | 416 [M + H] |
| 69 | 1.83 | 481 [M + H] |
| 70 | 3.02 | 636 [M + Na] |
| 71 | 3.03 | 469 [M + Na] |
| 72 | 3.01 | 468 [M + Na] |
| 73 | 3.21 | 470 [M + H] |
| 74 | 3.18 | 458 [M + Na] |
| 75 | 3.32 | 492 [M + Na] |
| 76 | 3.32 | 470 [M + H] |
| 77 | 3.08 | 484 [M + Na] |
| 78 | 3.29 | 492 [M + Na] |
| 79 | 3.05 | 462 [M + H] |
| 80 | 2.27 | 444 [M − H] |
| 81 | 2.21 | 418 [M − H] |
| 82 | 2.21 | 418 [M − H] |
| 83 | 2.16 | 474 [M − H] |
| 84 | 3.04 | 476 [M + H]] |
| 85 | 2.97 | 440 [M + Na] |
| 86 | 3.08 | 540 [M + H] |
| 87 | 3.25 | 510 [M + H] |
| 88 | 3.3 | 448 [M − H] |
| 89 | 3.37 | 485 [M + H] |
| 90 | 3.34 | 481 [M + H] |
| 91 | 3.28 | 481 [M + H] |
| 92 | 3.15 | 511 [M + H] |
| 93 | 3.42 | 530 [M + H] |
| 94 | 3.29 | 468 [M + H] |
| 95 | 3.39 | 485 [M + H] |
| 96 | 3.26 | 481 [M + H] |
| 97 | 3.39 | 465 [M + H] |
| 98 | 2.86 | 530 [M + H] |
| 99 | 3.23 | 495 [M + H] |
| 100 | 3.22 | 494 [M + H] |
| 101 | 3.41 | 518 [M + H] |
| 102 | 3.4 | 485 [M + H] |
| 103 | 3.49 | 519 [M + H] |
| 104 | 3.44 | 519 [M + H] |
| 105 | 3.31 | 511 [M + H] |
| 106 | 3.48 | 520 [M + H] |
| 107 | 3.27 | 511 [M + H] |
| 108 | 3.36 | 495 [M + H] |
| 109 | 3.3 | 468 [M + H] |
| 110 | 3.3 | 468 [M + H] |
| 111 | 3.24 | 525 [M + H] |
| 112 | 3.25 | 525 [M + H] |
| 113 | 3.2 | 488 [M + Na] |
| 114 | 3.28 | 589 [M + H] |
| 115 | 3.46 | 559 [M + H] |
| 116 | 3.23 | 430 [M + H] |
| 117 | 3.3 | 465 [M + H] |
| 118 | 3.27 | 460 [M + H] |
| 119 | 3.21 | 460 [M + H] |
| 120 | 3.06 | 490 [M + H] |
| 121 | 3.36 | 509 [M + H] |
| 122 | 3.23 | 448 [M + H] |
| 123 | 3.34 | 465 [M + H] |
| 124 | 3.19 | 460 [M + H] |
| 125 | 3.32 | 444 [M + H] |
| 126 | 2.79 | 509 [M + H] |
| 127 | 3.16 | 475 [M + H] |
| 128 | 3.14 | 474 [M + H] |
| 129 | 3.35 | 498 [M + H] |
| 130 | 3.33 | 465 [M + H] |
| 131 | 3.44 | 499 [M + H] |
| 132 | 3.38 | 499 [M + H] |
| 133 | 3.24 | 490 [M + H] |
| 134 | 3.42 | 499 [M + H] |
| 135 | 3.2 | 490 [M + H] |
| 136 | 3.29 | 474 [M + H] |
| 137 | 3.23 | 448 [M + H] |
| 138 | 3.23 | 448 [M + H] |
| 139 | 3.21 | 569 [M + H] |
| 140 | 3.4 | 539 [M + H] |
| 141 | 2.9 | 430.1 [M + H] |
| 142 | 2.93 | 460.1 [M + H] |
| 143 | 2.88 | 460.1 [M + H] |
| 144 | 2.78 | 490.1 [M + H] |
| 145 | 2.6 | 507.1 [M − H] |
| 146 | 2.9 | 448.1 [M + H] |
| 147 | 2.89 | 448.1 [M + H] |
| 148 | 2.85 | 504.1 [M + H] |
| 149 | 2.86 | 504.1 [M + H] |
| 150 | 2.75 | 432.1 [M + H] |
| 151 | 2.79 | 462.1 [M + H] |
| 152 | 2.78 | 462.1 [M + H] |
| 153 | 2.65 | 492.1 [M + H] |
| 154 | 2.43 | 511.1 [M + H] |
| 155 | 2.82 | 450.1 [M + H] |
| 156 | 2.77 | 450.1 [M + H] |
| 157 | 2.77 | 506.1 [M + H] |
| 158 | 2.74 | 506.1 [M + H] |
| 159 | 2.42 | 401.056 [M + H] |
| 160 | 2.71 | 416.056 [M + H] |
| 161 | 2.65 | 383.046 [M + H] |
| 162 | 2.77 | 435.077 [M + H] |
| 163 | 2.78 | 457.119 [M + H] |
| 164 | 2.98 | 441.124 [M + H] |
| 165 | 2.71 | 435.077 [M + H] |
| 166 | 2.99 | 441.124 [M + H] |
| 167 | 2.63 | 424.072 [M + H] |
| 168 | 2.51 | 415.072 [M + H] |
| 169 | 2.71 | 443.103 [M + H] |
| 170 | 2.66 | 455.103 [M + H] |
| 171 | 2.93 | 429.124 [M + H] |
| 172 | 2.84 | 401.093 [M + H] |
| 173 | 2.74 | 402.077 [M + H] |
| 174 | 2.88 | 415.108 [M + H] |
| 175 | 2.68 | 388.061 [M + H] |
| 176 | 2.94 | 443.14 [M + H] |
| 177 | 2.71 | 438.088 [M + H] |
| 178 | 2.96 | 441.124 [M + H] |
| 179 | 3.17 | 470.151 [M + H] |
| 180 | 2.56 | 402.04 [M + H] |
| 181 | 1.86 | 369 [M + H] |
| 182 | 2.11 | 384 [M + H] |
| 183 | 2.05 | 351 [M + H] |
| 184 | 1.77 | 403 [M + H] |
| 185 | 1.67 | 425 [M + H] |
| 186 | 1.71 | 409 [M + H] |
| 187 | 1.72 | 403 [M + H] |
| 188 | 1.72 | 409 [M + H]] |
| 189 | 1.66 | 392 [M + H] |
| 190 | 1.92 | 383 [M + H] |
| 191 | 1.66 | 411 [M + H] |
| 192 | 2.07 | 423 [M + H] |
| 193 | 1.73 | 397 [M + H] |
| 194 | 1.64 | 369 [M + H] |
| 195 | 2.14 | 370 [M + H] |
| 196 | 1.68 | 383 [M + H] |
| 197 | 2.09 | 356 [M + H] |
| 198 | 1.74 | 411 [M + H] |
| 199 | 1.69 | 406 [M + H] |
| 200 | 1.68 | 409 [M + H] |
| 201 | 1.60 | 438 [M + H] |
| 202 | 1.96 | 370 [M + H] |
| 203 | 2.29 | 383.1 [M + H] |
| 204 | 2.75 | 398.1 [M + H] |
| 205 | 2.6 | 417.1 [M + H] |
| 206 | 2.6 | 439.1 [M + H] |
| 207 | 2.79 | 423.1 [M + H] |
| 208 | 2.56 | 417.1 [M + H] |
| 209 | 2.83 | 423.1 [M + H] |
| 210 | 2.44 | 406.1 [M + H] |
| 211 | 2.51 | 423.1 [M − H] |
| 212 | 2.5 | 437.1 [M + H] |
| 213 | 2.73 | 411.1 [M + H] |
| 214 | 2.68 | 383.1 [M + H] |
| 215 | 2.57 | 384.1 [M + H] |
| 216 | 2.74 | 397.1 [M + H] |
| 217 | 2.5 | 370.1 [M + H] |
| 218 | 2.73 | 425.1 [M + H] |
| 219 | 2.53 | 420.1 [M + H] |
| 220 | 2.74 | 423.1 [M + H] |
| 221 | 2.89 | 452.2 [M + H] |
| 222 | 2.64 | 412.1 [M + H] |
| 223 | 2.69 | 431.1 [M + H] |
| 224 | 2.64 | 429.1 [M − H] |
| 225 | 2.51 | 420.1 [M + H] |
| 226 | 2.59 | 439.1 [M + H] |
| 227 | 2.68 | 398.1 [M + H] |
| 228 | 2.78 | 411.1 [M + H] |
| 229 | 2.61 | 384.1 [M + H] |
| 230 | 2.8 | 439.2 [M + H] |
| 231 | 2.61 | 434.1 [M + H] |
| 232 | 2.82 | 437.1 [M + H] |
| 233 | 2.46 | 414.1 [M + H] |
| 234 | 2.52 | 433.1 [M + H] |
| 235 | 2.5 | 433.1 [M + H] |
| 236 | 2.39 | 422.1 [M + H] |
| 237 | 2.45 | 441.1 [M + H] |
| 238 | 2.48 | 400.1 [M + H] |
| 239 | 2.6 | 413.1 [M + H] |
| 240 | 2.41 | 386.1 [M + H] |
| 241 | 2.68 | 441.1 [M + H] |
| 242 | 2.48 | 434.1 [M − H] |
| 243 | 2.68 | 439.1 [M + H] |
| 244 | 3.0 | 428 [M − H] |
| 245 | 3.2 | 442 [M − H] |
| 246 | 3.14 | 428.1 [M + H] |
| 247 | 3.11 | 428.1 [M + H] |
| 248 | 3.14 | 430.1 [M + H] |
| 249 | 3.17 | 446.1 [M + H] |
| 250 | 3.14 | 446.1 [M + H] |
| 251 | 3.18 | 448.1 [M + H] |
| 252 | 3.22 | 442.1 [M + H] |
| 253 | 3.19 | 442.1 [M + H] |
| 254 | 3.23 | 444.1 [M + H] |
| 255 | 3.27 | 462 [M + H] |
| 256 | 3.25 | 462 [M + H] |
| 257 | 3.28 | 464 [M + H] |
| 258 | 3.09 | 414.1 [M + H] |
| 259 | 3.07 | 414.1 [M + H] |
| 260 | 3.11 | 416.1 [M + H] |
| 261 | 3.06 | 448.097 [M + H] |
| 262 | 3.03 | 430.107 [M + H] |
| 263 | 2.99 | 416.091 [M + H] |
| 264 | 3.1 | 444.123 [M + H] |
| 265 | 3.16 | 464.068 [M + H] |
| 266 | 2.93 | 442.1 [M + H] |
| 267 | 2.9 | 440 [M − H] |
| 268 | 2.94 | 444.1 [M + H] |
| 269 | 2.8 | 444.1 [M + H] |
| 270 | 2.77 | 442.1 [M − H] |
| 271 | 2.81 | 446.1 [M + H] |
| 272 | 2.8 | 490 [M + H] |
| 273 | 3.1 | 538 [M + Na] |
| 274 | 3.2 | 514 [M + H] |
| 275 | 3.3 | 462 [M + H] |
| 276 | 3.4 | 488 [M + H] |
| 277 | 3.5 | 486 [M + H] |
| 278 | 3.1 | 430 [M + H] |
| 279 | 1.9 | 492 [M + H] |
| 280 | 1.7 | 480 [M + H] |
| 281 | 1.6 | 494 [M + H] |
| 282 | 1.65 | 478 [M + H] |
| 283 | 2.3 | 508 [M + H] |
| 284 | 3.3 | 444 [M + H] |
| 285 | 2.1 | 506 [M + H] |
| 286 | 1.83 | 494 [M + H] |
| 287 | 1.76 | 508 [M + H] |
| 288 | 1.8 | 492 [M + H] |
| 289 | 1.6 | 522 [M + H] |

### EXAMPLE 290

### 5-[(4-chlorophenyl)sulfonyl]-2-methyl-N-(2-morpholin-4-ylethyl)benzenesulfonamide

The title compound was prepared in a manner similar to that described in Example 1.

Step 1: 4-chlorophenyl-4-tolyl sulfone and chlorosulfonic acid were used to prepare 5-(4-chlorophenyl)sulfonyl-2-methylbenzenesulfonyl chloride.

Step 2: 5-(4-chlorophenyl)sulfonyl-2-methylbenzenesulfonyl chloride and 4-(2-aminoethyl)-morpholine were used to prepare 5-[(4-chlorophenyl)sulfonyl]-2-methyl-N-(2-moropholin-4-ylethyl)benzenesulfonamide.

MS (ES−) m/z 456.8;

HRMS: calcd for C₁₉H₂₃ClN₂O₅S₂+H+, 459.08097; found (ESI, [M+H]⁺), 459.0798.

### EXAMPLE 291

### 5-[(4-chlorophenyl)sulfonyl]-2-methyl-N-(2-pyridin-2-ylethyl)benzenesulfonamide

In an analogous manner to Example 290, 2-(2-aminoethyl)-pyridine was used to prepare 5-[(4-chlorophenyl)sulfonyl]-2-methyl-N-(2-pyridin-2-ylethyl)benzenesulfonamide.

MS (ES−) m/z 448.8;

HRMS: calcd C₂₀H₁₉ClN₂O₄S₂+H+, 451.05475; found (ESI, [M+H]⁺), 451.0546.

### EXAMPLE 292

### 5-[(4-chlorophenyl)sulfonyl]-N-[3-(1H-imidazol-1-yl)propyl]-2-methylbenzenesulfonamide

In an analogous manner to Example 290, 1-(3-aminopropyl)-imidazole was used to prepare 5-[(4-chlorophenyl)sulfonyl]-N-[3-(1H-imidazol-1-yl)propyl]-2-methylbenzenesulfonamide.

MS (ES−) m/z 451.8; HRMS: calcd for C₁₉H₂₀ClN₃O₄S₂+H+, 454.06565; found (ESI, [M+H]⁺), 454.0644.

### EXAMPLE 293

### 5-[(4-azidophenyl)sulfonyl]-N-[3-(1H-imidazol-1-yl)propyl]-2-methylbenzenesulfonamide

In an analogous manner to Example 353, 5-[(4-fluorophenyl)sulfonyl]-N-[3-(1H-imidazol-1-yl)propyl]-2-methylbenzenesulfonamide and sodium azide in DMF were used to prepare 5-[(4-azidophenyl)sulfonyl]-N-[3-(1H-imidazol-1-yl)propyl]-2-methylbenzenesulfonamide.

MS (ES−) m/z 458.8;

HRMS: calcd for C₁₉H₂₀N₆O₄S₂+H+, 461.10602; found (ESI, [M+H]⁺), 461.1066.

### EXAMPLE 294

### N-[2-(1H-indol-3-yl)ethyl]-2-methyl-5-(phenylsulfonyl)benzenesulfonamide

To a solution of 5-benzenesulfonyl-2-methyl-benzenesulfonyl chloride (0.36 g, 1.1 mmol) in DMF (10 mL) was added pyridine (0.13 mL, 1.6 mmol) and tryptamine (0.21 g, 1.3 mmol). The reaction was stirred at room temperature overnight and then diluted with ethyl acetate and washed with saturated ammonium chloride, saturated sodium bicarbonate, and brine. The organic layer was dried over magnesium sulfate, filtered and concentrated under reduced pressure. The crude residue was purified using automated flash chromatography with a gradient mobile phase consisting of ethyl acetate and hexane resulting in the isolation of N-[2-(1H-indol-3-yl)ethyl]-2-methyl-5-(phenylsulfonyl)benzenesulfonamide.

MS (ES−) m/z 452.8.

### EXAMPLE 295

### 2-isopropyl-N-(2-phenylethyl)-5-(phenylsulfonyl)benzenesulfonamide

The title compound was prepared in a manner similar to that described in Example 230.

Step 1: 4-isopropylbenzene sulfonyl chloride and benzene were used to prepare 1-benzenesulfonyl-4-isopropylbenzene, which was purified via Biotage Horizon™ (FLASH 40 M, silica, gradient from 100% hexane to 20% EtOAc/hexane).

Step 2: 1-Benzenesulfonyl-4-isopropylbenzene and chlorosulfonic acid were used to prepare 5-benzenesulfonyl-2-isopropyl-benzenesulfonyl chloride, which was purified via Biotage Horizon (FLASH 40 M, silica, gradient from 100% hexane to 20% EtOAc/hexane).

Step 3: 5-Benzenesulfonyl-2-isopropyl-benzenesulfonyl chloride and phenethyl amine were used to prepare 2-isopropyl-N-(2-phenylethyl)-5-(phenylsulfonyl)benzenesulfonamide, which was purified using a via Biotage Horizon™ (FLASH 25 M, silica, gradient from 10% hexane to 50% EtOAc/hexane gradient).

MS (ES−) m/z 441.9;

HRMS: calcd for C₂₃H₂₅NO₄S₂+H+, 444.12978; found (ESI, [M+H]⁺), 444.1297.

### EXAMPLE 296

### N-[3-(1H-imidazol-1-yl)propyl]-2-isopropyl-5-(phenylsulfonyl)benzenesulfonamide

In an analogous manner to Example 295:

Step 3: 5-Benzenesulfonyl-2-isopropyl-benzenesulfonyl chloride and 3-(1H-imidazol-1-yl)propylamine were used to prepare N-[3-(1H-imidazol-1-yl)propyl]-2-isopropyl-5-(phenylsulfonyl)benzenesulfonamide.

MS (ES−) m/z 445.9;

HRMS: calcd for C₂₁H₂₅N₃O₄S₂+H+, 448.13592; found (ESI, [M+H]⁺), 448.1364.

### EXAMPLE 297

### 2-ethyl-5-[(4-fluorophenyl)sulfonyl]-N-[2-(1H-imidazol-1-yl)ethyl]benzenesulfonamide

In an analogous manner to Example 295:

Step 1: 4-ethylbenzene sulfonyl chloride and fluorobenzene were used to prepare 1-ethyl-4-[(4-fluorophenyl)sulfonyl]benzene.

Step 2: 1-Ethyl-4-[(4-fluorophenyl)sulfonyl]benzene and chlorosulfonic acid were used to prepare 2-ethyl-5-(4-fluoro-benzenesulfonyl)-benzenesulfonyl chloride.

Step 3: 2-Ethyl-5-(4-fluoro-benzenesulfonyl)-benzenesulfonyl chloride and 2-(1H-imidazol-1-yl)ethylamine were used to prepare 2-ethyl-5-[(4-fluorophenyl)sulfonyl]-N-[2-(1H-imidazol-1-yl)ethyl]benzenesulfonamide.

MS (ES−) m/z 435.9;

HRMS: calcd for C₁₉H₂₀FN₃O₄S₂+H+, 438.09520; found (ESI, [M+H]⁺), 438.0945.

### EXAMPLE 298

### 2-methyl-5-(phenylsulfonyl)-N-tetrahydro-2H-pyran-4-ylbenzenesulfonamide

In an analogous manner to Example 1:

Step 2: 5-Benzenesulfonyl-2-methyl-benzenesulfonyl chloride and tetrahydro-pyran-4-ylamine were used to prepare 2-methyl-5-(phenylsulfonyl)-N-tetrahydro-2H-pyran-4-ylbenzenesulfonamide, which was purified using a via Biotage Horizon™ (FLASH 25 M, silica, gradient from 10% EtOAc/hexane to 50% EtOAc/hexane gradient.

MS (ESI+) m/z 396;

MS (ESI−) m/z 394;

HRMS: calcd for C₁₈H₂₁NO₅S₂+H+, 396.09339; found (ESI, [M+H]⁺), 396.0932.

### EXAMPLE 299

### 2-methyl-5-(phenylsulfonyl)-N-(2-tetrahydro-2H-pyran-4-ylethyl)benzenesulfonamide

In an analogous manner to Example 298:

Step 2: 5-Benzenesulfonyl-2-methyl-benzenesulfonyl chloride and 2-(tetrahydro-pyran-4-yl)-ethylamine were used to prepare 2-methyl-5-(phenylsulfonyl)-N-(2-tetrahydro-2H-pyran-4-ylethyl)benzenesulfonamide.

MS (ESI+) m/z 424;

MS (ESI−) m/z 422;

HRMS: calcd for C₂₀H₂₅NO₅S₂+H+, 424.12469; found (ESI, [M+H]⁺), 424.1237.

### EXAMPLE 300

### 2-ethyl-5-(phenylsulfonyl)-N-tetrahydro-2H-pyran-4-ylbenzenesulfonamide

In an analogous manner to Example 295:

Step 3: 5-benzenesulfonyl-2-ethyl-benzenesulfonyl chloride and tetrahydro-pyran-4-ylamine were used to prepare 2-ethyl-5-(phenylsulfonyl)-N-tetrahydro-2H-pyran-4-ylbenzenesulfonamide.

MS (ESI+) m/z 410;

MS (ESI−) m/z 408;

HRMS: calcd for C₁₉H₂₃NO₅S₂+H+, 410.10904; found (ESI, [M+H]⁺), 410.11.

### EXAMPLE 301

### 2-ethyl-5-(phenylsulfonyl)-N-(2-tetrahydro-2H-pyran-4-ylethyl)benzenesulfonamide

In an analogous manner to Example 295:

Step 3: 5-benzenesulfonyl-2-ethyl-benzenesulfonyl chloride and 2-(tetrahydro-pyran-4-yl)-ethylamine were used to prepare 2-ethyl-5-(phenylsulfonyl)-N-(2-tetrahydro-2H-pyran-4-ylethyl)benzenesulfonamide.

MS (ESI+) m/z 438;

MS (ESI−) m/z 436;

HRMS: calcd for C₂₁H₂₇NO₅S₂+H+, 438.14034; found (ESI, [M+H]⁺), 438.1385.

### EXAMPLE 302

### 2-methyl-N-(2-phenylethyl)-3-(phenylsulfonyl)benzenesulfonamide

To a stirred solution of 2-fluoro-6-nitrotoluene (5.0 g, 32.2 mmol) in DMF (80 mL) was added benzenethiol (4.0 mL, 38.8 mmol) and potassium carbonate (8.9 g, 64.4 mmol). The resulting solution was heated to 100° C. overnight and concentrated. The residue was taken up in ethyl acetate and washed with ammonium chloride solution (sat) and brine. The organic phase was dried over magnesium sulfate, concentrated and flash column separated using 3% ethyl acetate/hexane. The recovered material was dissolved in methylene chloride (70 mL) and m-chloroperbenzoic acid (8.0 g, 46.3 mmol) was added portionwise over 1 hour. The mixture was stirred an additional 30 minutes, washed with sodium bicarbonate solution (sat), dried over magnesium sulfate and concentrated. The residue was flash column separated using 20% ethyl acetate/hexane. The resulting material was added to methanol (30 mL), chilled to 0° C. and 10% palladium on carbon (120 mg) was added. Sodium borohydride (0.84 g, 22.2 mmol) was then added portionwise and stirred for 1 hour. The reaction was quenched with ammonium chloride solution (sat) and extracted several times with ethyl acetate. The organic layer was filtered through celite, washed with brine, dried over magnesium sulfate, and concentrated. The resulting material was dissolved in acetonitrile (16 mL), chilled to 0° C. and concentrated acetic acid (1.6 mL) and concentrated hydrochloric acid (1.6 mL) were added. Sodium nitrite (0.17 g, 2.42 mmol) dissolved in D.I. water (0.5 mL) was added dropwise and the solution was stirred 20 minutes. Sulfur dioxide was then bubbled into the solution over 20 minutes. Copper chloride dihydrate (0.34 g, 2.02 mmol) dissolved in D.I. water (0.5 mL) was added to the solution, the ice bath was removed and the solution was stirred at room temperature for 3.5 hours. The mixture was concentrated, taken up in ethyl acetate and washed with brine. The organic layer was dried over magnesium sulfate, and concentrated. The resulting material was dissolved in THF (4 mL), triethylamine (0.4 mL, 2.86 mmol) and phenethylamine (0.1 mL, 0.79 mmol) were added and the mixture was stirred at room temperature for 30 minutes. The solution was treated with sodium bicarbonate solution (sat) and extracted several times with ethyl acetate. The combined organic layers were dried over magnesium sulfate and concentrated to give 2-methyl-N-(2-phenylethyl)-3-(phenylsulfonyl)benzenesulfonamide (0.05 g, 5%).

MS (ES−) m/z 413.9;

HRMS: calcd for C₂₁H₂₁NO₄S₂+H+, 416.09848; found (ESI, [M+H]⁺), 416.0975.

### EXAMPLE 303

### 2-ethyl-5-[(4-fluorophenyl)sulfonyl]-N-[3-(1H-imidazol-1-yl)propyl]benzenesulfonamide

In an analogous manner to Example 295:

Step 3: 2-Ethyl-5-(4-fluoro-benzenesulfonyl)-benzenesulfonyl chloride and 3-(1H-imidazol-1-yl)propylamine were used to prepare 2-ethyl-5-[(4-fluorophenyl)sulfonyl]-N-[2-(1H-imidazol-1 -yl)propyl]benzenesulfonamide.

### EXAMPLE 304

### 5-({4-[ethyl(methyl)amino]phenyl}sulfonyl)-N-[3-(1H-imidazol-1-yl)propyl]-2-methylbenzenesulfonamide

In an analogous manner to Example 353, 5-[(4-fluorophenyl)sulfonyl]-N-[3-(1H-imidazol-1-yl)propyl]-2-methylbenzenesulfonamide and N-ethylmethylamine were used to prepare 5-({4-[ethyl(methyl)amino]phenyl}sulfonyl)-N-[3-(1H-imidazol-1-yl)propyl]-2-methylbenzenesulfonamide.

MS (ES−) m/z 474.9;

HRMS: calcd for C₂₂H₂₈N₄O₄S₂+H+, 477.16247; found (ESI, [M+H]⁺), 477.1625.

### EXAMPLE 305

### N-[3-(1H-imidazol-1-yl)propyl]-2-methyl-5-[(4-pyrrolidin-1-ylphenyl)sulfonyl]benzenesulfonamide

In an analogous manner to Example 353, 5-[(4-fluorophenyl)sulfonyl]-N-[3-(1H-imidazol-1-yl)propyl]-2-methylbenzenesulfonamide and pyrrolidine were used to prepare N-[3-(1H-imidazol-1-yl)propyl]-2-methyl-5-[(4-pyrrolidin-1-ylphenyl)sulfonyl]benzenesulfonamide.

MS (ES−) m/z 486.9;

HRMS: calcd for C₂₃H₂₈N₄O₄S₂+H+, 489.16247; found (ESI, [M+H]⁺), 489.1643.

### EXAMPLE 306

### N-[3-(1H-imidazol-1-yl)propyl]-4-methyl-3-(phenylsulfinyl)benzenesulfonamide

In an analogous manner to Example 302, 2-fluoro-4-nitrotoluene and 1 equivalent of m-chloroperbenzoic acid were used to prepare N-[3-(1H-imidazol-1-yl)propyl-4-methyl-3-(phenylsulfinyl)benzenesulfonamide.

MS (ESI+) m/z 404;

HRMS: calcd for C₁₉H₂₁N₃O₃S₂+H+, 404.10971; found (ESI, [M+H]⁺), 404.1116.

### EXAMPLE 307

### 5-[(4-fluorophenyl)sulfonyl]-N-[2-(1H-imidazol-1-yl)ethyl]-2-methylbenzenesulfonamide

In an analogous manner to Example 298:

Step 2: 2-Methyl-5-(4-fluoro-benzenesulfonyl)-benzenesulfonyl chloride and 2-(1H-imidazol-1-yl)ethylamine were used to prepare 5-[(4-fluorophenyl)sulfonyl]-N-[2-(1H-imidazol-1-yl)ethyl]-2-methylbenzenesulfonamide.

MS (ES+) m/z 424;

MS (ES−) m/z 422;

HRMS: calcd for C₁₈H₁₈FN₃O₄S₂+H+, 424.07955; found (ESI, [M+H]⁺), 424.0798.

### EXAMPLE 308

### 5-[(4-fluorophenyl)sulfonyl]-2-methyl-N-tetrahydro-2H-pyran-4-ylbenzenesulfonamide

In an analogous manner to Example 298:

Step 2: 2-Methyl-5-(4-fluoro-benzenesulfonyl)-benzenesulfonyl chloride and tetrahydro-pyran-4-ylamine were used to prepare 5-[(4-fluorophenyl)sulfonyl]-2-methyl-N-tetrahydro-2H-pyran-4-ylbenzenesulfonamide.

MS (ES+) m/z 414;

MS (ES−) m/z 412;

HRMS: calcd for C₁₈H₂₀FNO₅S₂+H+, 414.08397; found (ESI, [M+H]⁺), 414.0843.

### EXAMPLE 309

### 5-[(4-fluorophenyl)sulfonyl]-2-methyl-N-(2-tetrahydro-2H-pyran-4-ylethyl)benzenesulfonamide

In an analogous manner to Example 298:

Step 2: 2-Methyl-5-(4-fluoro-benzenesulfonyl)-benzenesulfonyl chloride and 2-(tetrahydro-pyran-4-yl)-ethylamine were used to prepare 5-[(4-fluorophenyl)sulfonyl]-2-methyl-N-(2-tetrahydro-2H-pyran-4-ylethyl)benzenesulfonamide.

MS (ES+) m/z 442;

MS (ES−) m/z 440;

HRMS: calcd for C₂₀H₂₄FNO₅S₂+H+, 442.11527; found (ESI, [M+H]⁺), 442.1167.

### EXAMPLE 310

### N-[3-(1H-imidazol-1-yl)propyl]-2-methyl-5-{[4-(methylamino)phenyl]sulfonyl}benzenesulfonamide

In an analogous manner to Example 356, 5-[(4-fluorophenyl)sulfonyl]-N-[3-(1H-imidazol-1-yl)propyl]-2-methylbenzenesulfonamide and methylamine were used to prepare N-[3-(1H-imidazol-1-yl)propyl]-2-methyl-5-{[4-(methylamino)phenyl]sulfonyl}benzenesulfonamide.

MS (ES−) m/z 446.9;

HRMS: calcd for C₂₀H₂₄N₄O₄S₂+H+, 449.13117; found (ESI, [M+H]⁺), 449.1319.

### EXAMPLE 311

### 5-{[4-(ethylamino)phenyl]sulfonyl}-N-[3-(1H-imidazol-1-yl)propyl]-2-methylbenzenesulfonamide

In an analogous manner to Example 356, 5-[(4-fluorophenyl)sulfonyl]-N-[3-(1H-imidazol-1-yl)propyl]-2-methylbenzenesulfonamide and ethylamine (70% aqueous solution) were used to prepare 5-{[4-(ethylamino)phenyl]sulfonyl}-N-[3-(1H-imidazol-1-yl)propyl]-2-methylbenzenesulfonamide.

MS (ES−) m/z 460.9;

HRMS: calcd for C₂₁H₂₆N₄O₄S₂+H+, 463.14682; found (ESI, [M+H]⁺), 463.1487.

### EXAMPLE 312

### N-[3-(1H-imidazol-1-yl)propyl]-2-methyl-5-[(4-piperidin-1-ylphenyl)sulfonyl]benzenesulfonamide

In an analogous manner to Example 353, 5-[(4-fluorophenyl)sulfonyl]-N-[3-(1H-imidazol-1-yl)propyl]-2-methylbenzenesulfonamide and piperidine were used to prepare N-[3-(1H-imidazol-1-yl)propyl]-2-methyl-5-[(4-piperidin-1-ylphenyl)sulfonyl]benzenesulfonamide.

MS (ES+) m/z 503;

MS (ES−) m/z 501;

HRMS: calcd for C₂₄H₃₀N₄O₄S₂+H+, 503.17812; found (ESI, [M+H]⁺), 503.1782.

### EXAMPLE 313

### N-[3-(1H-imidazol-1-yl)propyl]-2-methyl-5-[(4-morpholin-4-ylphenyl)sulfonyl]benzenesulfonamide

In an analogous manner to Example 353, 5-[(4-fluorophenyl)sulfonyl]-N-[3-(1H-imidazol-1-yl)propyl]-2-methylbenzenesulfonamide and morpholine were used to prepare N-[3-(1H-imidazol-1-yl)propyl]-2-methyl-5-[(4-morpholin-4-ylphenyl)sulfonyl]benzenesulfonamide.

MS (ES+) m/z 505;

MS (ES−) m/z 503;

HRMS: calcd for C₂₃H₂₈N₄O₅S₂+H+, 505.15739; found (ESI, [M+H]⁺), 505.1587.

### EXAMPLE 314

### N-[2-(1H-imidazol-1-yl)ethyl]-2-methyl-5-(phenylsulfonyl)benzenesulfonamide

In an analogous manner to Example 298:

Step 2: 5-Benzenesulfonyl-2-methyl-benzenesulfonyl chloride and 2-(1H-imidazol-1-yl)ethylamine were used to prepare N-[2-(1H-imidazol-1-yl)ethyl]-2-methyl-5-(phenylsulfonyl)benzenesulfonamide.

MS (ES−) m/z 403.8;

HRMS: calcd for C₁₈H₁₉N₃O₄S₂+H+, 406.08897; found (ESI, [M+H]⁺), 406.0878.

### EXAMPLE 315

### N-[3-(1H-imidazol-1-yl)propyl]-5-[(4-methoxyphenyl)sulfonyl]-2-methylbenzenesulfonamide

Step 1: To a stirred solution of 5-bromo-2-methylaniline (1.49 g, 8.01 mmol) in acetonitrile (65 mL) at 0° C. was added glacial acetic acid (6.5 mL) and concentrated HCl (6.5 mL). A solution of sodium nitrite (0.66 g, 9.61 mmol) dissolved in D.I water (2 mL) was added dropwise. The resulting solution was stirred 20 minutes. Sulfur dioxide was then bubbled into the solution for 20 minutes. A solution of copper (II) chloride dihydrate (1.37 g, 8.01 mmol) dissolved in D.I. water (2 mL) was added, the ice bath was removed and the solution was allowed to stir overnight at room temperature. The reaction volume was concentrated to one third and extracted several times with ethyl acetate. The organic layers were combined, washed with water and sat. sodium bicarbonate solution, dried over magnesium sulfate and concentrated. The crude was dissolved in THF (22 mL) and triethylamine (1.8 mL, 12.91 mmol) was added. 1-(3-aminopropyl)-imidazole (1.0 mL, 8.38 mmol) was added dropwise and the resulting solution was stirred 30 minutes at room temperature. The solution was partitioned between an aqueous ammonium chloride solution and ethyl acetate. The organic layer was dried over magnesium sulfate and concentrated to give 5-bromo-N-[3-(1H-imidazol-1-yl)propyl]-2-methylbenzenesulfonamide (1.41 g, 49%).

MS (ES) m/z 355.8;

HRMS: calcd for C₁₃H₁₆BrN₃O₂S+H+, 358.02193; found (ESI, [M+H]⁺), 358.0211.

Step 2: A solution of nickel (II) bromide (0.03 g, 0.11 mmol), zinc powder (0.03 g, 0.45 mmol), 1,1′-bis(diphenylphosphino)-ferrocene (0.12 g, 0.22 mmol), and potassium carbonate (0.31 g, 2.24 mmol) in NMP (10 mL) was stirred at room temperature for 1 hour. 5-bromo-N-[3-(1H-imidazol-1-yl)propyl]-2-methylbenzenesulfonamide (0.70 g, 1.95 mmol) and 4-methoxybenzenethiol (0.2 mL, 1.61 mmol) were added and the resulting solution was heated overnight at 80° C. The solution was partitioned between an aqueous ammonium chloride solution and ethyl acetate. The organic layer was concentrated and HPLC separated. The resulting solid was dissolved in methylene chloride (5 mL). M-chloroperbenzoic acid (0.29 g, 77%, 1.29 mmol) was added and the solution was stirred 1 hour at room temperature, concentrated and HPLC separated to give N-[3-(1H-imidazol-1-yl)propyl]-5-[(4-methoxyphenyl)sulfonyl]-2-methylbenzenesulfonamide (0.11 g, 13%).

MS (ES−) m/z 447.9;

HRMS: calcd for C₂₀H₂₃N₃O₅S₂+H+, 450.11519; found (ESI, [M+H]⁺), 450.1148.

### EXAMPLE 316

### N-[3-(1H-imidazol-1-yl)propyl]-5-[(2-methoxyphenyl)sulfonyl]-2-methylbenzenesulfonamide

In an analogous manner to Example 315:

Step 2: 2-methoxybenzenethiol was used to prepare N-[3-(1H-imidazol-1-yl)propyl]-5-[(2-methoxyphenyl)sulfonyl]-2-methylbenzenesulfonamide.

MS (ES+) m/z 450;

MS (ES−) m/z 448;

HRMS: calcd for C₂₀H₂₃N₃O₅S₂+H+, 450.11519; found (ESI, [M+H]⁺), 450.1148.

### EXAMPLE 317

### N-[3-(1H-imidazol-1-yl)propyl]-4-methyl-3-(phenylsulfonyl)benzenesulfonamide

To a stirred solution of N-[3-(1H-imidazol-1-yl)propyl]-4-methyl-3-(phenylsulfinyl)benzenesulfonamide (0.13 g, 0.32 mmol) in methylene chloride (7 mL) was added m-chloroperbenzoic acid (0.05 g, 0.32 mmol). The solution was then concentrated and HPLC separated to give N-[3-(1H-imidazol-1-yl)propyl]-4-methyl-3-(phenylsulfonyl)benzenesulfonamide. (0.004 g, 3%).

MS (ES+) m/z 420;

MS (ES−) m/z 418.

### EXAMPLE 318

### 5-[(4-fluorophenyl)sulfonyl]-N-[2-(1H-imidazol-1-yl)ethyl]-2-isopropylbenzenesulfonamide

In an analogous manner to Example 295:

Step 1: 4-isopropylbenzene sulfonyl chloride and fluorobenzene were used to prepare 1-isopropyl-4-[(4-fluorophenyl)sulfonyl]benzene, which was purified via Biotage Horizon™ (FLASH 40 M, silica, gradient from 100% hexane to 20% EtOAc/hexane).

Step 2: 1-Isopropyl-4-[(4-fluorophenyl)sulfonyl]benzene and chlorosulfonic acid were used to prepare 2-isopropyl-5-(4-fluoro-benzenesulfonyl)-benzenesulfonyl chloride, which was purified via Biotage Horizon™ (FLASH 40 M, silica, gradient from 100% hexane to 20% EtOAc/hexane).

Step 3: 2-Isopropyl-5-(4-fluoro-benzenesulfonyl)-benzenesulfonyl chloride and 2-(1H-imidazol-1-yl)ethylamine were used to prepare 5-[(4-fluorophenyl)sulfonyl]-N-[2-(1H-imidazol-1-yl)ethyl]-2-isopropylbenzenesulfonamide.

MS (ES−) m/z 449.8;

HRMS: calcd for C₂₀H₂₂FN₃O₄S₂+H+, 452.11085; found (ESI, [M+H]⁺), 452.1094.

### EXAMPLE 319

### 5-[(4-fluorophenyl)sulfonyl]-N-[3-(1H-imidazol-1-yl)propyl]-2-isopropylbenzenesulfonamide

In an analogous manner to Example 295:

Step 3: 2-Isopropyl-5-(4-fluoro-benzenesulfonyl)-benzenesulfonyl chloride and 3-(1H-imidazol-1-yl)propylamine were used to prepare 5-[(4-fluorophenyl)sulfonyl]-N-[3-(1H-imidazol-1-yl)propyl]-2-isopropylbenzenesulfonamide.

MS (ES−) m/z 463.8;

HRMS: calcd for C₂₁H₂₄FN₃O₄S₂+H+, 466.12650; found (ESI, [M+H]⁺), 466.1253.

### EXAMPLE 320

### 5-[(4-fluorophenyl)sulfonyl]-2-isopropyl-N-(2-pyridin-2-ylethyl)benzenesulfonamide

In an analogous manner to Example 295:

Step 3: 2-Isopropyl-5-(4-fluoro-benzenesulfonyl)-benzenesulfonyl chloride and 2-pyridin-2-yl-ethylamine were used to prepare 5-[(4-fluorophenyl)sulfonyl]-2-isopropyl-N-(2-pyridin-2-ylethyl)benzenesulfonamide.

MS (ES−) m/z 460.8;

HRMS: calcd for C₂₂H₂₃FN₂O₄S₂+H+, 463.11560; found (ESI, [M+H]⁺), 463.115.

### EXAMPLE 321

### 5-[(4-fluorophenyl)sulfonyl]-2-isopropyl-N-tetrahydro-2H-pyran-4-ylbenzenesulfonamide

In an analogous manner to Example 295:

Step 3: 2-Isopropyl-5-(4-fluoro-benzenesulfonyl)-benzenesulfonyl chloride and tetrahydro-pyran-4-ylamine were used to prepare 5-[(4-fluorophenyl)sulfonyl]-2-isopropyl-N-tetrahydro-2H-pyran-4-ylbenzenesulfonamide.

MS (ES−) m/z 439.9;

HRMS: calcd for C₂₀H₂₄FNO₅S₂+H+, 442.11527; found (ESI, [M+H]⁺), 442.1174.

### EXAMPLE 322

### 5-[(4-fluorophenyl)sulfonyl]-2-isopropyl-N-(2-tetrahydro-2H-pyran-4-ylethyl)benzenesulfonamide

In an analogous manner to Example 295:

Step 3: 2-Isopropyl-5-(4-fluoro-benzenesulfonyl)-benzenesulfonyl chloride and 2-(tetrahydro-pyran-4-yl)-ethylamine were used to prepare 5-[(4-fluorophenyl)sulfonyl]-2-isopropyl-N-(2-tetrahydro-2H-pyran-4-ylethyl)benzenesulfonamide.

MS (ES−) m/z 467.9;

HRMS: calcd for C₂₂H₂₈FNO₅S₂+H+, 470.14657; found (ESI, [M+H]⁺), 470.1471;

### EXAMPLE 323

### N-[3-(1H-imidazol-1-yl)propyl]-5-[(3-methoxyphenyl)sulfonyl]-2-methylbenzenesulfonamide

In an analogous manner to Example 315:

Step2: 3-methoxybenzenethiol was used to prepare N-[3-(1H-imidazol-1-yl)propyl]-5-[(3-methoxyphenyl)sulfonyl]-2-methylbenzenesulfonamide.

MS (ES−) m/z 447.9;

HRMS: calcd for C₂₀H₂₃N₃O₅S₂+H+, 450.11519; found (ESI, [M+H]⁺), 450.1154.

### EXAMPLE 324

### N-[2-(1H-imidazol-1-yl)ethyl]-2,4-dimethyl-5-(phenylsulfonyl)benzenesulfonamide

In an analogous manner to Example 295:

Step 1: Benzene sulfonyl chloride and meta xylene were used to prepare 1-benzenesulfonyl-2,4-dimethyl-benzene, which was purified via Biotage Horizon™ (FLASH 40 M, silica, gradient from 100% hexane to 20% EtOAc/hexane).

Step 2: 1-Benzenesulfonyl-2,4-dimethyl-benzene and chlorosulfonic acid were used to prepare 5-benzenesulfonyl-2,4-dimethyl-benzenesulfonyl chloride, which was purified via Biotage Horizon™ (FLASH 40 M, silica, gradient from 100% hexane to 20% EtOAc/hexane).

Step 3: 5-Benzenesulfonyl-2,4-dimethyl-benzenesulfonyl chloride and 2-(1H-imidazol-1-yl)ethylamine were used to prepare N-[2-(1H-imidazol-1-yl)ethyl]-2,4-dimethyl-5-(phenylsulfonyl)benzenesulfonamide.

MS (ESI+) m/z 420;

MS (ESI−) m/z 418;

HRMS: calcd for C₁₉H₂₁N₃O₄S₂+H+, 420.10462; found (ESI, [M+H]⁺), 420.1051.

### EXAMPLE 325

### N-[3-(1H-imidazol-1-yl)propyl]-2,4-dimethyl-5-(phenylsulfonyl)benzenesulfonamide

In an analogous manner to Example 298:

Step 2: 5-Benzenesulfonyl-2,4-dimethyl-benzenesulfonyl chloride and 3-(1H-imidazol-1-yl)propylamine were used to prepare N-[3-(1H-imidazol-1-yl)propyl]-2,4-dimethyl-5-(phenylsulfonyl)benzenesulfonamide.

MS (ESI+) m/z 434;

MS (ESI−) m/z 432;

HRMS: calcd for C₂₀H23N₃O₄S₂+H+, 434.12027; found (ESI, [M+H]⁺), 434.1186.

### EXAMPLE 326

### 2,4-dimethyl-5-(phenylsulfonyl)-N-(2-pyridin-2-ylethyl)benzenesulfonamide

In an analogous manner to Example 298:

Step 2: 5-Benzenesulfonyl-2,4-dimethyl-benzenesulfonyl chloride and 2-pyridin-2-yl-ethylamine were used to prepare 2,4-dimethyl-5-(phenylsulfonyl)-N-(2-pyridin-2-ylethyl)benzenesulfonamide.

MS (ES−) m/z 428.9;

HRMS: calcd for C₂₁H₂₂N₂O₄S₂+H+, 431.10937; found (ESI, [M+H]⁺), 431.1079.

### EXAMPLE 327

### 2,4-dimethyl-5-(phenylsulfonyl)-N-tetrahydro-2H-pyran-4-ylbenzenesulfonamide

In an analogous manner to Example 298:

Step 2: 5-Benzenesulfonyl-2,4-dimethyl-benzenesulfonyl chloride and tetrahydro-pyran-4-ylamine were used to prepare 2,4-dimethyl-5-(phenylsulfonyl)-N-tetrahydro-2H-pyran-4-ylbenzenesulfonamide.

MS (ES−) m/z 407.9;

HRMS: calcd for C₁₉H₂₃NO₅S₂+H+, 410.10904; found (ESI, [M+H]⁺), 410.1096.

### EXAMPLE 328

### 2,4-dimethyl-5-(phenylsulfonyl)-N-(2-tetrahydro-2H-pyran-4-ylethyl)benzenesulfonamide

In an analogous manner to Example 298:

Step 2: 5-Benzenesulfonyl-2,4-dimethyl-benzenesulfonyl chloride and 2-(tetrahydro-pyran-4-yl)-ethylamine were used to prepare 2,4-dimethyl-5-(phenylsulfonyl)-N-(2-tetrahydro-2H-pyran-4-ylethyl)benzenesulfonamide.

MS (ES−) m/z 435.9;

HRMS: calcd for C₂₁H₂₇NO₅S₂+H+, 438.14034; found (ESI, [M+H]⁺), 438.141.

### EXAMPLE 329

### 5-[(4-fluorophenyl)sulfonyl]-N-[2-(1H-imidazol-1-yl)ethyl]-2,4-dimethylbenzenesulfonamide

In an analogous manner to Example 295:

Step 1: 4-Fluorobenzene sulfonyl chloride and meta xylene were used to prepare 1-(4-fluoro-benzenesulfonyl)-2,4-dimethyl-benzene, which was purified via Biotage Horizon™ (FLASH 40 M, silica, gradient from 100% hexane to 20% EtOAc/hexane).

Step 2: 1-(4-Fluoro-benzenesulfonyl)-2,4-dimethyl-benzene and chlorosulfonic acid were used to prepare 5-(4-fluoro-benzenesulfonyl)-2,4-dimethyl-benzenesulfonyl chloride, which was purified via Biotage Horizon™ (FLASH 40 M, silica, gradient from 100% hexane to 20% EtOAc/hexane).

Step 3: 5-(4-Fluoro-benzenesulfonyl)-2,4-dimethyl-benzenesulfonyl chloride and 2-(1H-imidazol-1-yl)ethylamine were used to prepare 5-[(4-fluorophenyl)sulfonyl]-N-[2-(1H-imidazol-1-yl)ethyl]-2,4-dimethylbenzenesulfonamide.

MS (ESI+) m/z 438;

MS (ESI−) m/z 436;

HRMS: calcd for C₁₉H₂₀FN₃O₄S₂+H+, 438.09520; found (ESI, [M+H]⁺), 438.0945.

### EXAMPLE 330

### 5-[(4-fluorophenyl)sulfonyl]-N-[3-(1H-imidazol-1-yl)propyl]-2,4-dimethylbenzenesulfonamide

In an analogous manner to Example 298:

Step 2: 5-(4-Fluoro-benzenesulfonyl)-2,4-dimethyl-benzenesulfonyl chloride and 3-(1H-imidazol-1-yl)propylamine were used to prepare 5-[(4-fluorophenyl)sulfonyl]-N-[3-(1H-imidazol-1-yl)propyl]-2,4-dimethylbenzenesulfonamide.

MS (ESI+) m/z 452;

MS (ESI−) m/z 450;

HRMS: calcd for C₂₀H₂₂FN₃O₄S₂+H+, 452.11085; found (ESI, [M+H]⁺), 452.1115.

### EXAMPLE 331

### 5-[(4-fluorophenyl)sulfonyl]-2,4-dimethyl-N-(2-pyridin-2-ylethyl)benzenesulfonamide

In an analogous manner to Example 298:

Step 2: 5-(4-Fluoro-benzenesulfonyl)-2,4-dimethyl-benzenesulfonyl chloride and 2-pyridin-2-yl-ethylamine were used to prepare 5-[(4-fluorophenyl)sulfonyl]-2,4-dimethyl-N-(2-pyridin-2-ylethyl)benzenesulfonamide.

MS (ESI+) m/z 449;

MS (ESI−) m/z 447;

HRMS: calcd for C₂₁H₂₁FN₂O₄S₂+H+, 449.09995; found (ESI, [N+H]⁺), 449.0979.

### EXAMPLE 332

### 5-[(4-fluorophenyl)sulfonyl]-2,4-dimethyl-N-(tetrahydro-2H-pyran-4-yl)benzenesulfonamide

In an analogous manner to Example 298:

Step 2: 5-(4-Fluoro-benzenesulfonyl)-2,4-dimethyl-benzenesulfonyl chloride and tetrahydro-pyran-4-ylamine were used to prepare 5-[(4-fluorophenyl)sulfonyl]-2,4-dimethyl-N-(tetrahydro-2H-pyran-4-yl)benzenesulfonamide.

MS (ESI+) m/z 428;

MS (ESI−) m/z 426;

HRMS: calcd for C₁₉H₂₂FNO₅S₂+H+, 428.09962; found (ESI, [M+H]⁺), 428.0981.

### EXAMPLE 333

### 5-[(4-fluorophenyl)sulfonyl]-2,4-dimethyl-N-[2-(tetrahydro-2H-pyran-4-yl)ethyl]benzenesulfonamide

In an analogous manner to Example 298:

Step 2: 5-(4-Fluoro-benzenesulfonyl)-2,4-dimethyl-benzenesulfonyl chloride and 2-(tetrahydro-pyran-4-yl)-ethylamine were used to prepare 5-[(4-fluorophenyl)sulfonyl]-2,4-dimethyl-N-[2-(tetrahydro-2H-pyran-4-yl)ethyl]benzenesulfonamide.

MS (ESI+) m/z 456;

MS (ESI−) m/z 454;

HRMS: calcd for C₂₁H₂₆FNO₅S₂+H+, 456.13092; found (ESI, [M+H]⁺), 456.1321.

### EXAMPLE 334

### 2-chloro-N-(2-phenylethyl)-5-(phenylsulfonyl)benzenesulfonamide

Step 1: 3-nitro-4-chlorobenzenesulfonylchloride (5.0 g, 19.5 mmol) was added portionwise to a stirred solution of aluminum chloride (3.12 g, 23.4 mmol) in benzene (10 mL) and stirred overnight at room temperature. The solution was poured over ice and extracted several times with ethyl acetate. The combined organic layers were dried over magnesium sulfate and concentrated. The crude solid was dissolved in methanol (100 mL) and water (3 mL). Tin (II) chloride (11.0 g, 58.0 mmol) was added and the resulting solution was heated to 70° C. overnight. The solution was concentrated and partitioned between ethyl acetate and a saturated sodium bicarbonate solution. The organic layer was dried over magnesium sulfate and concentrated to give [2-chloro-5-(phenylsulfonyl)phenyl]amine (2.59 g, 36%).

MS (ESI+) m/z 268;

HRMS: calcd for C₁₂H₁₀ClNO₂S +H+, 268.01935; found (ESI, [M+H]⁺), 268.0202;

Step 2: To a stirred solution of [2-chloro-5-(phenylsulfonyl)phenyl]amine (2.5 g, 9.34 mmol) in acetonitrile (75 mL) at 0° C. was added glacial acetic acid (7.5 mL) and concentrated HCl (7.5 mL). A solution of sodium nitrite (0.77 g, 11.21 mmol) dissolved in D.I water (3 mL) was added dropwise. The resulting solution was stirred 20 minutes. Sulfur dioxide was then bubbled into the solution for 20 minutes. A solution of copper (II) chloride dihydrate (1.59 g, 9.34 mmol) dissolved in D.I. water (3 mL) was added, the ice bath was removed and the solution was allowed to stir overnight at room temperature. The reaction volume was concentrated to one third and extracted several times with ethyl acetate. The organic layers were combined, washed with water and sat. sodium bicarbonate solution, dried over magnesium sulfate and concentrated. The crude material was carried on without further purification.

Step 3: Following the same procedure described in Example 1 (step 2), 5-benzenesulfonyl-2-chlorobenzenesulfonyl chloride and phenethylamine were used to prepare 2-chloro-N-(2-phenylethyl)-5-(phenylsulfonyl)benzenesulfonamide.

MS (ES−) m/z 433.8;

HRMS: calcd for C₂₀H₁₈ClNO₄S₂+H+, 436.04385; found (ESI, [M+H]⁺), 436.0475.

### EXAMPLE 335

### 2-chloro-N-(2-morpholin-4-ylethyl)-5-(phenylsulfonyl)benzenesulfonamide

In an analogous manner to Example 334:

Step 3: 4-(2-aminoethyl)-morpholine was used to prepare 2-chloro-N-(2-morpholin-4-ylethyl)-5-(phenylsulfonyl)benzenesulfonamide.

MS (ES−) m/z 442.8;

HRMS: calcd for C₁₈H₂₁ClN₂O₅S₂+H+, 445.06532; found (ESI, [M+H]⁺), 445.0679.

### EXAMPLE 336

### 2-chloro-N-[3-(1H-imidazol-1-yl)propyl]-5-(phenylsulfonyl)benzenesulfonamide

In an analogous manner to Example 334:

Step 3: 1-(3-aminopropyl)-imidazole was used to prepare 2-chloro-N-[3-(1H-imidazol-1-yl)propyl]-5-(phenylsulfonyl)benzenesulfonamide.

MS (ES−) m/z 437.8;

HRMS: calcd for C₁₈H₁₈ClN₃O₄S₂+H+, 440.05000; found (ESI, [M+H]⁺), 440.0482.

### EXAMPLE 337

### 2-chloro-5-(phenylsulfonyl)-N-(2-pyridin-2-ylethyl)benzenesulfonamide

In an analogous manner to Example 334:

Step 3: 2-(2-aminoethyl)-pyridine was used to prepare 2-chloro-5-(phenylsulfonyl)-N-(2-pyridin-2-ylethyl)benzenesulfonamide.

MS (ES−) m/z 434.8;

HRMS: calcd for C₁₉H₁₇ClN₂O₄S₂+H+, 437.03910; found (ESI, [M+H]⁺), 437.042.

### EXAMPLE 338

### N-[3-(1H-imidazol-1-yl)propyl]-5-[(4-isopropylphenyl)sulfonyl]-2-methylbenzenesulfonamide

In an analogous manner to Example 315:

Step 2: 4-isopropylbenzenethiol was used to prepare N-[3-(1H-imidazol-1-yl)propyl]-5-[(4-isopropylphenyl)sulfonyl]-2-methylbenzenesulfonamide.

MS (ES−) m/z 460.0;

HRMS: calcd for C₂₂H₂₇N₃O₄S₂+H+, 462.15157; found (ESI, [M+H]⁺), 462.1525.

### EXAMPLE 339

### N-[3-(1H-imidazol-1-yl)propyl]-2-methyl-5-(2-naphthylsulfonyl)benzenesulfonamide

In an analogous manner to Example 315:

Step 2: 2-naphthlyene was used to prepare N-[3-(1H-imidazol-1-yl)propyl]-2-methyl-5-(2-naphthylsulfonyl)benzenesulfonamide.

MS (ES−) m/z 467.9;

HRMS: calcd for C₂₃H₂₃N₃O₄S₂+H+, 470.12027; found (ESI, [M+H]⁺), 470.1189.

### EXAMPLE 340

### 5-[(3,4-dichlorophenyl)sulfonyl]-N-[3-(1H-imidazol-1-yl)propyl]-2-methylbenzenesulfonamide

Step 1: In an analogous manner to Example 315, step 1.

Step 2: A solution of nickel (II) bromide (0.03 g, 0.11 mmol), zinc powder (0.03 g, 0.45 mmol), 1,1′-bis(diphenylphosphino)-ferrocene (0.12 g, 0.22 mmol), and potassium carbonate (0.31 g, 2.24 mmol) in NMP (8 mL) was stirred at room temperature for 1 hour. 5-bromo-N-[3-(1H-imidazol-1-yl)propyl]-2-methylbenzenesulfonamide (0.70 g, 1.95 mmol) and 3,4-dichlorobenzenethiol (0.2 mL,1.57 mmol) were added and the resulting solution was heated in the microwave at 160° C. for 10 minutes. The solution was partitioned between a saturated aqueous ammonium chloride solution and ethyl acetate. The organic layer was concentrated and HPLC separated. The resulting solid was dissolved in methylene chloride (5 mL). M-chloroperbenzoic acid (0.22 g, 77%, 0.99 mmol) was added and the solution was stirred 1 hour at room temperature, washed with a saturated aqueous sodium dithionite solution, a saturated aqueous sodium bicarbonate solution, concentrated and HPLC separated to give 5-[(3,4-dichlorophenyl)sulfonyl]-N-[3-(1H-imidazol-1-yl)propyl]-2-methylbenzenesulfonamide (0.08 g, 10%).

MS (ES−) m/z 485.8;

HRMS: calcd for C₁₉H₁₉Cl₂N₃O₄S₂+H+, 488.02668; found (ESI, [M+H]⁺), 488.03.

### EXAMPLE 341

### 2-(2-hydroxy-2-methylpropyl)-N-(2-phenylethyl)-5-(phenylsulfonyl)benzenesulfonamide

To a solution of 2-methyl-N-(2-phenylethyl)-5-(phenylsulfonyl)benzenesulfonamide (1.0 g 2.4 mmol) in tetrahydrofuran (20 mL) was added butyllithium (6.0 mL of 2.5 M in hexanes) dropwise at −78° C. The solution was allowed to stir at −78° C. for twenty minutes and then acetone (0.18 mL, 2.4 mmol) was added dropwise. The reaction was stirred at −78° C. for one hour and then poured onto ice/water and extracted with ethyl acetate (3×75 mL). The combined organics were dried with magnesium sulfate and concentrated down. The crude residue was purified using automated flash chromatography with a gradient mobile phase consisting of ethyl acetate and hexane resulting in the isolation of 2-(2-hydroxy-2-methylpropyl)-N-(2-phenylethyl)-5-(phenylsulfonyl)benzenesulfonamide (0.12 g, 11%).

MS (ES+) m/z 474;

MS (ES−) m/z 472;

HRMS: calcd. for C₂₄H₂₇NO₅S₂+H+, 474.1409: found (ESI, [m+H]⁺), 474.1397.

### EXAMPLE 342

### 5-[(3-chlorophenyl)sulfonyl]-N-[3-(1H-imidazol-1-yl)propyl]-2-methylbenzenesulfonamide

In an analogous manner to Example 340:

Step 2: 3-chlorobenzenethiol was used to prepare 5-[(3-chlorophenyl)sulfonyl]-N-[3-(1H-imidazol-1-yl)propyl]-2-methylbenzenesulfonamide.

HRMS: calcd for C₁₉H₂₀ClN₃O₄S₂+H+, 454.06565; found (ESI, [M+H]⁺), 454.0649.

### EXAMPLE 343

### 5-[(3,5-dimethylphenyl)sulfonyl]-N-[3-(1H-imidazol-1-yl)propyl]-2-methylbenzenesulfonamide

In an analogous manner to Example 340:

Step 2: 3,5-dimethylbenzenethiol was used to prepare 5-[(3,5-dimethylphenyl)sulfonyl]-N-[3-(1H-imidazol-1-yl)propyl]-2-methylbenzenesulfonamide.

MS (ESI+) m/z 448;

HRMS: calcd for C₂₁H₂₅N₃O₄S₂+H+, 448.13592; found (ESI, [M+H]⁺), 448.1387.

### EXAMPLE 344

### 5-[(3,5-dichlorophenyl)sulfonyl]-N-[3-(1H-imidazol-1-yl)propyl]-2-methylbenzenesulfonamide

In an analogous manner to Example 340:

Step2: 3,5-dichlorobenzenethiol was used to prepare 5-[(3,5-dichlorophenyl)sulfonyl]-N-[3-(1H-imidazol-1-yl)propyl]-2-methylbenzenesulfonamide.

HRMS: calcd for C₁₉H₁₉Cl₂N₃O₄S₂+H+, 488.02668; found (ESI, [M+H]⁺), 488.0278.

### EXAMPLE 345

### 5-[(2,5-dichlorophenyl)sulfonyl]-N-[3-(1H-imidazol-1-yl)propyl]-2-methylbenzenesulfonamide

In an analogous manner to Example 377:

Step 2: 2,5-dichlorobenzenethiol was used to prepare 5-[(2,5-dichlorophenyl)sulfonyl]-N-[3-(1H-imidazol-1-yl)propyl]-2-methylbenzenesulfonamide.

MS (ES−) m/z 485.8;

HRMS: calcd for C₁₉H₁₉Cl₂N₃O₄S₂+H+, 488.02668; found (ESI, [M+H]⁺), 488.0279.

### EXAMPLE 346

### N-[3-(1H-imidazol-1-yl)propyl]-2-methyl-5-(phenylsulfinyl)benzenesulfonamide

In an analogous manner to Example 340:

Step 2: benzenethiol and 1 equivalent of m-chloroperbenzoic acid were used to prepare N-[3-(1H-imidazol-1-yl)propyl]-2-methyl-5-(phenylsulfinyl)benzenesulfonamide.

MS (ES−) m/z 402.0;

HRMS: calcd for C₁₉H₂₁N₃O₃S₂+H+, 404.10971; found (ESI, [M+H]⁺), 404.1118.

### EXAMPLE 347

### N-[2-(1H-imidazol-1-yl)ethyl]-2,3-dimethyl-5-(phenylsulfonyl)benzenesulfonamide

In an analogous manner to Example 295:

Step 1: Benzene sulfonyl chloride and ortho xylene were used to prepare 4-Benzenesulfonyl-1,2-dimethyl-benzene, which was purified via Biotage Horizon™ (FLASH 40 M, silica, gradient from 100% hexane to 20% EtOAc/hexane).

Step 2: 4-Benzenesulfonyl-1,2-dimethyl-benzene and chlorosulfonic acid were used to prepare 5-benzenesulfonyl-2,3-dimethyl-benzenesulfonyl chloride, which was purified via Biotage Horizon™ (FLASH 40 M, silica, gradient from 100% hexane to 20% EtOAc/hexane).

Step 3: 5-Benzenesulfonyl-2,3-dimethyl-benzenesulfonyl chloride and 2-(1H-imidazol-1-yl)ethylamine were used to prepare N-[2-(1H-imidazol-1-yl)ethyl]-2,3-dimethyl-5-(phenylsulfonyl)benzenesulfonamide.

HRMS: calcd for C₁₉H₂₁N₃O₄S₂+H+, 420.10462; found (ESI, [M+H]⁺), 420.1036.

### EXAMPLE 348

### N-[3-(1H-imidazol-1-yl)propyl]-2,3-dimethyl-5-(phenylsulfonyl)benzenesulfonamide

In an analogous manner to Example 298:

Step 2: 5-Benzenesulfonyl-2,3-dimethyl-benzenesulfonyl chloride and 3-(1H-imidazol-1-yl)propylamine were used to prepare N-[3-(1H-imidazol-1-yl)propyl]-2,3-dimethyl-5-(phenylsulfonyl)benzenesulfonamide.

HRMS: calcd for C₂₀H₂₃N₃O₄S₂+H+, 434.12027; found (ESI, [M+H]⁺), 434.1187.

### EXAMPLE 349

### 2,3-dimethyl-5-(phenylsulfonyl)-N-(2-pyridin-2-ylethyl)benzenesulfonamide

In an analogous manner to Example 298:

Step 2: 5-Benzenesulfonyl-2,3-dimethyl-benzenesulfonyl chloride and 2-pyridin-2-yl-ethylamine were used to prepare 2,3-dimethyl-5-(phenylsulfonyl)-N-(2-pyridin-2-ylethyl)benzenesulfonamide.

HRMS: calcd for C₂₁H₂₂N₂O₄S₂+H+, 431.10937; found (ESI, [M+H]⁺), 431.1085.

### EXAMPLE 350

### 2,3-dimethyl-5-(phenylsulfonyl)-N-(tetrahydro-2H-pyran-4-yl)benzenesulfonamide

In an analogous manner to Example 298:

Step 2: 5-Benzenesulfonyl-2,3-dimethyl-benzenesulfonyl chloride and tetrahydro-pyran-4-ylamine were used to prepare 2,3-dimethyl-5-(phenylsulfonyl)-N-(tetrahydro-2H-pyran-4-yl)benzenesulfonamide.

HRMS: calcd for C₁₉H₂₃NO₅S₂+H+, 410.1096; found (ESI, [M+H]⁺), 410.1114.

### EXAMPLE 351

### 2,3-dimethyl-5-(phenylsulfonyl)-N-[2-(tetrahydro-2H-pyran-4-yl)ethyl]benzenesulfonamide

In an analogous manner to Example 298:

Step 2: 5-Benzenesulfonyl-2,3-dimethyl-benzenesulfonyl chloride and 2-(tetrahydro-pyran-4-yl)-ethylamine were used to prepare 2,3-dimethyl-5-(phenylsulfonyl)-N-[2-(tetrahydro-2H-pyran-4-yl)ethyl]benzenesulfonamide.

HRMS: calcd for C₂₁H₂₇NO₅S₂+H+, 438.14034; found (ESI, [M+H]⁺), 438.1413.

### EXAMPLE 352

### 5-{[4-(cyclohexylamino)phenyl]sulfonyl}-N-[3-(1H-imidazol-1-yl)propyl]-2-methylbenzenesulfonamide

In an analogous manner to Example 353, 5-[(4-fluorophenyl)sulfonyl]-N-[3-(1H-imidazol-1-yl)propyl]-2-methylbenzenesulfonamide and cyclohexylamine were used to prepare 5-{[4-(cyclohexylamino)phenyl]sulfonyl}-N-[3-(1H-imidazol-1-yl)propyl]-2-methylbenzenesulfonamide.

MS (ES−) m/z 515.0;

HRMS: calcd for C₂₅H₃₂N₄O₄S₂+H+, 517.19377; found (ESI, [M+H]⁺), 517.1782.

### EXAMPLE 353

### 5-({4-[(2-cyanoethyl)amino]phenyl}sulfonyl)-N-[3-(1H-imidazol-1-yl)propyl]-2-methylbenzenesulfonamide

To a solution of 5-[(4-fluorophenyl)sulfonyl]-N-[3-(1H-imidazol-1-yl)propyl]-2-methylbenzenesulfonamide (75 mg, 0.17 mmol) in N,N-dimethylacetamide (0.5 mL) was added 3-aminopropionitrile (67 mg, 0.96 mmol). The solution was heated with stirring using microwave irradiation (180° C.) for 65 minutes. Upon cooling, the reaction mixture was diluted with water (10 mL) and the aqueous phase was extracted with ethyl acetate (3×10 mL). The combined organic phases were concentrated and the resulting crude residue was purified using automated flash column chromatography with a graduated mobile phase consisting of dichloromethane and methanol resulting in the isolation of 5-({4-[(2-cyanoethyl)amino]phenyl}sulfonyl)-N-[3-(1H-imidazol-1-yl)propyl]-2-methylbenzenesulfonamide (38 mg, 45%).

MS (ES+) m/z 488.0

MS (ES−) m/z 485.9

HRMS: calcd for C₂₂H25N₅O₄S₂+H+, 488.1426; found (ESI, [M+H]⁺), 488.1440.

### EXAMPLE 354

### 5-[(4-{[(1S,2S)-1-(hydroxymethyl)-2-methylbutyl]amino}phenyl)sulfonyl]-N-[3-(1H-imidazol-1-yl)propyl]-2-methylbenzenesulfonamide

In an analogous manner to Example 353, 5-[(4-fluorophenyl)sulfonyl]-N-[3-(1H-imidazol-1-yl)propyl]-2-methylbenzenesulfonamide and (1S,2S)-1-(hydroxymethyl)-2-methylbutyl]amine were used to prepare 5-[(4-{(1S,2S)-1-(hydroxymethyl)-2-methylbutyl]amino}phenyl)sulfonyl]-N-[3-(1H-imidazol-1-yl)propyl]-2-methylbenzenesulfonamide.

MS (ES−) m/z 533.0;

HRMS: calcd for C₂₅H₃₄N₄O₅S₂+H+, 535.20434; found (ESI, [M+H]⁺), 535.2024.

### EXAMPLE 355

### N-[3-(1H-imidazol-1-yl)propyl]-2-methyl-5-({4-[(1-phenylethyl)amino]phenyl}sulfonyl)benzenesulfonamide

In an analogous manner to Example 353, 5-[(4-fluorophenyl)sulfonyl]-N-[3-(1H-imidazol-1-yl)propyl]-2-methylbenzenesulfonamide and 2-phenylethylamine were used to prepare N-[3-(1H-imidazol-1-yl)propyl]-2-methyl-5-({4-[(1-phenylethyl)amino]phenyl}sulfonyl)benzenesulfonamide.

MS (ES−) m/z 536.9;

HRMS: calcd for C₂₇H₃₀N₄O₄S₂+H+, 539.17812; found (ESI, [M+H]⁺), 539.1788.

### EXAMPLE 356

### 2-methyl-5-{[4-(methylamino)phenyl]sulfonyl)-N-(2-pyridin-2-ylethyl)benzenesulfonamide

To a solution of 5-[(4-fluorophenyl)sulfonyl]-2-methyl-N-(2-pyridin-2-ylethyl)benzene-sulfonamide (75 mg, 0.17 mmol) in N,N-dimethylacetamide (0.5 mL) was added an ethanolic solution containing methyl amine (33%, 0.5 mL). The solution was heated with stirring using microwave irradiation (180° C.) for 20 minutes. Upon cooling, the reaction mixture was diluted with water (10 mL) and the aqueous phase was extracted with ethyl acetate (3×10 mL). The combined organic phases were concentrated and the resulting crude residue was purified using automated flash column chromatography with a graduated mobile phase consisting of dichloromethane and methanol resulting in the isolation of 2-methyl-5-{[4-(methylamino)phenyl]sulfonyl}-N-(2-pyridin-2-ylethyl)-benzenesulfonamide (92 mg, 81%).

MS (ES−) m/z 446;

HRMS: calcd for C₂₁H₂₃N₃O₄S₂+H+, 446.12027; found (ESI, [M+H]⁺), 446.1209.

### EXAMPLE 357

### 2-isopropyl-5-{[4-(methylamino)phenyl]sulfonyl}-N-(2-pyridin-2-ylethyl)benzenesulfonamide

In an analogous manner to Example 356, 5-[(4-fluorophenyl)sulfonyl]-2-isopropyl-N-(2-pyridin-2-ylethyl)benzenesulfonamide and methylamine were used to prepare N-[3-(1H-imidazol-1-yl)propyl]-2-methyl-5-{[4-(methylamino)phenyl]sulfonyl}benzenesulfonamide.

MS (ES+) m/z 474;

MS (ES−) m/z 472;

HRMS: calcd for C₂₃H₂₇N₃O₄S₂+H+, 474.15157; found (ESI, [M+H]⁺), 474.1511.

### EXAMPLE 358

### 2-isopropyl-5-{[4-(methylamino)phenyl]sulfonyl}-N-(tetrahydro-2H-pyran-4-yl)benzenesulfonamide

In an analogous manner to Example 356, 5-[(4-fluorophenyl)sulfonyl]-2-isopropyl-N-tetrahydro-2H-pyran-4-ylbenzenesulfonamide and methylamine were used to prepare 2-isopropyl-5-{[4-(methylamino)phenyl]sulfonyl}-N-(tetrahydro-2H-pyran-4-yl)benzenesulfonamide.

MS (ES+) m/z 453;

MS (ES−) m/z 451;

HRMS: calcd for C₂₁H₂₈N₂O₅S₂+H+, 453.15124; found (ESI, [M+H]⁺), 453.1505.

### EXAMPLE 359

### 2-isopropyl-5-{[4-(methylamino)phenyl]sulfonyl}-N-[2-(tetrahydro-2H-pyran-4-yl)ethyl]benzenesulfonamide

In an analogous manner to Example 356, 5-[(4-fluorophenyl)sulfonyl]-2-isopropyl-N-(2-tetrahydro-2H-pyran-4-ylethyl)benzenesulfonamide and methylamine were used to prepare 2-isopropyl-5-{[4-(methylamino)phenyl]sulfonyl}-N-[2-(tetrahydro-2H-pyran-4-yl)ethyl]benzenesulfonamide.

MS (ES+) m/z 481;

MS (ES−) m/z 479;

HRMS: calcd for C₂₃H₃₂N₂O₅S₂+H+, 481.18254; found (ESI, [M+H]⁺), 481.1837.

### EXAMPLE 360

### 5-[(2,3-dichlorophenyl)sulfonyl]-N-[3-(1H-imidazol-1-yl)propyl]-2-methylbenzenesulfonamide

In an analogous manner to Example 340:

Step 2: 2,3-dichlorobenzenethiol was used to prepare 5-[(2,3-dichlorophenyl)sulfonyl]-N-[3-(1H-imidazol-1-yl)propyl]-2-methylbenzenesulfonamide.

MS (ESI+) m/z 488;

MS (ESI−) m/z 486;

HRMS: calcd for C₁₉H₁₉Cl₂N₃O₄S₂+H+, 488.02668; found (ESI, [M+H]⁺), 488.0269.

### EXAMPLE 361

### 2-chloro-5-(phenylsulfonyl)-N-(tetrahydro-2H-pyran-4-yl)benzenesulfonamide

In an analogous manner to Example 334:

Step 3: tetrahydropyran-4-ylamine was used to prepare 2-chloro-5-(phenylsulfonyl)-N-(tetrahydro-2H-pyran-4-yl)benzenesulfonamide.

MS (ESI−) m/z 414;

HRMS: calcd for C₁₇H₁₈ClNO₅S₂+H+, 416.03877; found (ESI, [M+H]⁺), 416.0392.

### EXAMPLE 362

### N-[3-(1H-imidazol-1-yl)propyl]-2-methyl-5-(2-thienylsulfonyl)benzenesulfonamide

In an analogous manner to Example 340:

Step 2: thiophene-2-thiol was used to prepare N-[3-(1H-imidazol-1-yl)propyl]-2-methyl-5-(2-thienylsulfonyl)benzenesulfonamide.

MS (ESI+) m/z 426;

HRMS: calcd for C₁₇H₁₉N₃O₄S₃+H+, 426.06104; found (ESI, [M+H]⁺), 426.062.

### EXAMPLE 363

### N-[3-(1H-imidazol-1-yl)propyl]-2-methyl-5-[(2-methyl-3-furyl)sulfonyl]benzenesulfonamide

In an analogous manner to Example 340:

Step 2: 2-methylfuran-3-thiol was used to prepare N-[3-(1H-imidazol-1-yl)propyl]-2-methyl-5-[(2-methyl-3-furyl)sulfonyl]benzenesulfonamide.

MS (ESI+) m/z 424;

MS (ESI−) m/z 422;

HRMS: calcd for C₁₈H₂₁N₃O₅S₂+H+, 424.09954; found (ESI, [M+H]⁺), 424.0983.

### EXAMPLE 364

### N-{[2-methyl-5-(phenylsulfonyl)phenyl]sulfonyl}-L-phenylalaninamide

In an analogous manner to Example 294, 5-benzenesulfonyl-2-methyl-benzenesulfonyl chloride, diisopropylethylamine, and L-phenylalaninamide in dichloromethane were used to prepare N-{[2-methyl-5-(phenylsulfonyl)phenyl]sulfonyl}-L-phenylalaninamide.

MS (ESI+) m/z 459

MS (ESI−) m/z 457.

### EXAMPLE 365

### methyl N-{[2-methyl-5-(phenylsulfonyl)phenyl]sulfonyl}-D-phenylalaninate

In an analogous manner to Example 294, 5-benzenesulfonyl-2-methyl-benzenesulfonyl chloride, diisopropylethylamine, and D-phenylalanine methyl ester in dichloromethane were used to prepare methyl N-{[2-methyl-5-(phenylsulfonyl)phenyl]sulfonyl}-D-phenylalaninate.

MS (ESI+) m/z 474;

MS (ESI−) m/z 472.

### EXAMPLE 366

### N-[3-(1H-imidazol-1-yl)propyl]-2-methyl-5-{[4-(tetrahydro-2H-pyran-4-ylamino)phenyl]sulfonyl}benzenesulfonamide

In an analogous manner to Example 353, 5-[(4-fluorophenyl)sulfonyl]-N-[3-(1H-imidazol-1-yl)propyl]-2-methylbenzenesulfonamide and 4-aminotetrahydropyran were used to prepare N-[3-(1H-imidazol-1-yl)propyl]-2-methyl-5-{[4-(tetrahydro-2H-pyran-4-ylamino)phenyl]sulfonyl}benzenesulfonamide.

MS (ESI+) m/z 519;

MS (ESI−) m/z 517;

HRMS: calcd for C₂₄H₃₀N₄O₅S₂+H+, 519.17304; found (ESI, [M+H]⁺), 519.1755.

### EXAMPLE 367

### N-[3-(1H-imidazol-1-yl)propyl]-5-({4-[(3-isopropoxypropyl)amino]phenyl}sulfonyl)-2-methylbenzenesulfonamide

In an analogous manner to Example 353, 5-[(4-fluorophenyl)sulfonyl]-N-[3-(1H-imidazol-1-yl)propyl]-2-methylbenzenesulfonamide and 3-isopropoxypropyl)amine were used to prepare N-[3-(1H-imidazol-1-yl)propyl]-5-({4-[(3-isopropoxypropyl)amino]phenyl}sulfonyl)-2-methylbenzenesulfonamide.

MS (ESI+) m/z 535;

MS (ESI−) m/z 533;

HRMS: calcd for C₂₅H₃₄N₄O₅S₂+H+, 535.20434; found (ESI, [M+H]⁺), 535.2054.

### EXAMPLE 368

### 5-({4-[(cyclopropylmethyl)amino]phenyl}sulfonyl)-N-[3-(1H-imidazol-1-yl)propyl]-2-methylbenzenesulfonamide

In an analogous manner to Example 353, 5-[(4-fluorophenyl)sulfonyl]-N-[3-(1H-imidazol-1-yl)propyl]-2-methylbenzenesulfonamide and cyclopropylmethylamine were used to prepare 5-({4-[(cyclopropylmethyl)amino]phenyl}sulfonyl)-N-[3-(1H-imidazol-1-yl)propyl]-2-methylbenzenesulfonamide.

MS (ESI+) m/z 489;

MS (ESI−) m/z 487;

HRMS: calcd for C₂₃H₂₈N₄O₄S₂+H+, 489.16247; found (ESI, [M+H]⁺), 489.1653.

### EXAMPLE 369

### 5-({4-[(1R,2R,4S)-bicyclo[2.2.1]hept-2-ylamino]phenyl}sulfonyl)-N-[3-(1H-imidazol-1-yl)propyl]-2-methylbenzenesulfonamide

In an analogous manner to Example 353, 5-[(4-fluorophenyl)sulfonyl]-N-[3-(1H-imidazol-1-yl)propyl]-2-methylbenzenesulfonamide and (1R,2R,4S)-bicyclo[2.2.1]hept-2-ylamine were used to prepare 5-({4-[(1R,2R,4S)-bicyclo[2.2.1]hept-2-ylamino]phenyl}sulfonyl)-N-[3-(1H-imidazol-1-yl)propyl]-2-methylbenzenesulfonamide.

MS (ESI+) m/z 529;

MS (ESI−) m/z 527;

HRMS: calcd for C₂₆H₃₂N₄O₄S₂+H+, 529.19377; found (ESI, [M+H]⁺), 529.1946.

### EXAMPLE 370

### 5-{[4-(benzylamino)phenyl]sulfonyl}-N-[3-(1H-imidazol-1-yl)propyl]-2-methylbenzenesulfonamide

In an analogous manner to Example 353, 5-[(4-fluorophenyl)sulfonyl]-N-[3-(1H-imidazol-1-yl)propyl]-2-methylbenzenesulfonamide and benzylamine were used to prepare 5-{[4-(benzylamino)phenyl]sulfonyl)-N-[3-(1H-imidazol-1-yl)propyl]-2-methylbenzenesulfonamide.

MS (ESI+) m/z 525;

MS (ESI−) m/z 523.

### EXAMPLE 371

### 5-[(4-{[(1S)-1-cyclohexylethyl]amino}phenyl)sulfonyl]-N-[3-(1H-imidazol-1-yl)propyl]-2-methylbenzenesulfonamide

In an analogous manner to Example 353, 5-[(4-fluorophenyl)sulfonyl]-N-[3-(1H-imidazol-1-yl)propyl]-2-methylbenzenesulfonamide and (1S)-1-cyclohexylethylamine were used to prepare 5-[(4-{[(1S)-1-cyclohexylethyl]amino}phenyl)sulfonyl]-N-[3-(1H-imidazol-1-yl)propyl]-2-methylbenzenesulfonamide.

MS (ESI+) m/z 545;

HRMS: calcd for C₂₇H₃₆N₄O₄S₂+H+, 545.22507; found (ESI, [M+H]⁺), 545.2244.

### EXAMPLE 372

### 5-[(4-{[(1R)-1-cyclohexylethyl]amino}phenyl)sulfonyl]-N-[3-(1H-imidazol-1-yl)propyl]-2-methylbenzenesulfonamide

In an analogous manner to Example 353, 5-[(4-fluorophenyl)sulfonyl]-N-[3-(1H-imidazol-1-yl)propyl]-2-methylbenzenesulfonamide and (1R)-1-cyclohexylethylamine were used to prepare 5-[(4-{[(1R)-1-cyclohexylethyl]amino}phenyl)sulfonyl]-N-[3-(1H-imidazol-1-yl)propyl]-2-methylbenzenesulfonamide.

MS (ESI+) m/z 545;

MS (ESI−) m/z 543;

HRMS: calcd for C₂₇H₃₆N₄O₄S₂+H+, 545.22507; found (ESI, [M+H]⁺), 545.2255.

### EXAMPLE 373

### 5-({4-[(2-hydroxybutyl)amino]phenyl}sulfonyl)-N-[3-(1H-imidazol-1-yl)propyl]-2-methylbenzenesulfonamide

In an analogous manner to Example 353, 5-[(4-fluorophenyl)sulfonyl]-N-[3-(1H-imidazol-1-yl)propyl]-2-methylbenzenesulfonamide and 2-hydroxybutylamine were used to prepare 5-({4-[(2-hydroxybutyl)amino]phenyl}sulfonyl)-N-[3-(1H-imidazol-1-yl)propyl]-2-methylbenzenesulfonamide.

MS (ESI+) m/z 507;

MS (ESI−) m/z 505;

HRMS: calcd for C₂₃H₃₀N₄O₅S₂+H+, 507.17304; found (ESI, [M+H]⁺), 507.1738.

### EXAMPLE 374

### N-[3-(1H-imidazol-1-yl)propyl]-2-methyl-5-[(4-{[4-(trifluoromethyl)benzyl]amino}phenyl)sulfonyl]benzenesulfonamide

In an analogous manner to Example 353, 5-[(4-fluorophenyl)sulfonyl]-N-[3-(1H-imidazol-1-yl)propyl]-2-methylbenzenesulfonamide and 4-(trifluoromethyl)benzylamine were used to prepare N-[3-(1H-imidazol-1-yl)propyl]-2-methyl-5-[(4-{[4-(trifluoromethyl)benzyl]amino}phenyl)sulfonyl]benzenesulfonamide.

MS (ESI+) m/z 593;

HRMS: calcd for C₂₇H₂₇F₃N₄O₄S₂+H+, 593.14986; found (ESI, [M+H]⁺), 593.1508.

### EXAMPLE 375

### 5-[(4-bromophenyl)sulfonyl]-2-methyl-N-(tetrahydro-2H-pyran-4-yl)benzenesulfonamide

In an analogous manner to Example 230:

Step 1: p-toluene sulfonyl chloride and bromobenzene were used to prepare 1-bromo-4-[(4-methylphenyl)sulfonyl]benzene.

Step 2: Following the same procedure described in Example 1 (step 1), 1-bromo-4-[(4-methylphenyl)sulfonyl]benzene and chlorosulfonic acid was used to prepare 5-(4-bromobenzenesulfonyl)-2-methylbenzenesulfonyl chloride.

Step 3: Following the same procedure described in Example 1 (step 2), 5-(4-bromobenzenesulfonyl)-2-methylbenzenesulfonyl chloride and tetrahydropyran-4-ylamine were used to prepare 5-[(4-bromophenyl)sulfonyl]-2-methyl-N-(tetrahydro-2H-pyran-4-yl)benzenesulfonamide.

MS (ESI−) m/z 472;

### EXAMPLE 376

### 5-[(4-cyanophenyl)sulfonyl]-2-methyl-N-(tetrahydro-2H-pyran-4-yl)benzenesulfonamide

To a stirred solution of 5-[(4-bromophenyl)sulfonyl]-2-methyl-N-(tetrahydro-2H-pyran-4-yl)benzenesulfonamide (0.30 g, 0.63 mmol) in DMF (3 mL) was added zinc cyanide (0.05 g, 0.38 mmol), tris(dibenzylideneacetone) dipalladium(0) (0.03 g, 0.03 mmol), and 1,1′-bis(diphenylphosphino)-ferrocene (0.04 g, 0.06 mmol). The resulting solution was heated to reflux for 1 hour. The solution was concentrated. Flash column separation with 10%-60% ethyl acetate/hexane followed by trituration gave 5-[(4-cyanophenyl)sulfonyl]-2-methyl-N-(tetrahydro-2H-pyran-4-yl)benzenesulfonamide (0.11 g, 42%).

MS (ESI−) m/z 419.

### EXAMPLE 377

### 5-[(2-chlorophenyl)sulfonyl]-N-[3-(1H-imidazol-1-yl)propyl]-2-methylbenzenesulfonamide

Step 1: In an analogous manner to Example 315, step 1.

Step 2: A solution of nickel (II) bromide (0.03 g, 0.11 mmol), zinc powder (0.03 g, 0.45 mmol), 1,1′-bis(diphenylphosphino)-ferrocene (0.12 g, 0.22 mmol), and potassium carbonate (0.31 g, 2.24 mmol) in NMP (8 mL) was stirred at room temperature for 1 hour. 5-bromo-N-[3-(1H-imidazol-1-yl)propyl]-2-methylbenzenesulfonamide (0.70 g, 1.95 mmol) and 2-chlorobenzenethiol (0.18 mL,1.57 mmol) were added and the resulting solution was heated in the microwave at 160° C. for 10 minutes. The solution was partitioned between a saturated aqueous ammonium chloride solution and ethyl acetate. The organic layer was concentrated and HPLC separated. The resulting solid was dissolved in methylene chloride (5 mL). Oxone (0.68 g, 1.10 mmol) was added and the solution was stirred 2 days at room temperature, washed with a saturated aqueous ammonium chloride solution, dried over magnesium sulfate and concentrated to give 5-[(2-chlorophenyl)sulfonyl]-N-[3-(1H-imidazol-1-yl)propyl]-2-methylbenzenesulfonamide (0.04 g, 4%).

MS (ESI+) m/z 454;

MS (ESI−) m/z 452;

HRMS: calcd for C₁₉H₂₀ClN₃O₄S₂+H+, 454.06565; found (ESI, [M+H]⁺), 454.0683.

### EXAMPLE 378

### N-[3-(1H-imidazol-1-yl)propyl]-2-methyl-5-(pyridin-2-ylsulfonyl)benzenesulfonamide

In an analogous manner to Example 340:

Step 2: 2-mercaptopyridine was used to prepare N-[3-(1H-imidazol-1-yl)propyl]-2-methyl-5-(pyridin-2-ylsulfonyl)benzenesulfonamide.

MS (ESI+) m/z 421;

HRMS: calcd for C₁₈H₂₀N₄O₄S₂+H+, 421.09987; found (ESI, [M+H]⁺), 421.0989.

### EXAMPLE 379

### 5-[(2,4-dichlorophenyl)sulfonyl]-N-[3-(1H-imidazol-1-yl)propyl]-2-methylbenzenesulfonamide

In an analogous manner to Example 377:

Step 2: 2,4-dichlorobenzenethiol was used to prepare 5-[(2,4-dichlorophenyl)sulfonyl]-N-[3-(1H-imidazol-1-yl)propyl]-2-methylbenzenesulfonamide.

MS (ESI+) m/z 488;

MS (ESI−) m/z 486;

HRMS: calcd for C₁₉H₁₉Cl₂N₃O₄S₂+H+, 488.02668; found (ESI, [M+H]⁺), 488.0299.

### EXAMPLE 380

### 5-[(4-acetylphenyl)sulfonyl]-2-methyl-N-(tetrahydro-2H-pyran-4-yl)benzenesulfonamide

To a stirred mixture of 5-[(4-bromophenyl)sulfonyl]-2-methyl-N-(tetrahydro-2H-pyran-4-yl)benzenesulfonamide (0.70 g, 1.46 mmol) in toluene (10 mL) was added tetrakis(triphenylphosphine) palladium(0) (0.01 g, 0.07 mmol), and tributyl(1-ethoxyvinyl)tin (0.5 mL, 1.48 mmol). The resulting solution was heated at 100° C. overnight and partitioned between a saturated aqueous ammonium chloride solution and ethyl acetate. The organic layer was concentrated, taken up in THF (10 mL) and 2N HCl (5 mL) was added. The resulting solution was stirred at room temperature for 2 hours and extracted several times with ethyl acetate. The combined organic layers were filtered through celite and concentrated. Trituration with ether gave 5-[(4-acetylphenyl)sulfonyl]-2-methyl-N-(tetrahydro-2H-pyran-4-yl)benzenesulfonamide (0.54 g, 85%).

MS (ESI+) m/z 438;

MS (ESI−) m/z 436;

HRMS: calcd for C₂₀H₂₃NO₆S₂+H+, 438.10396; found (ESI, [M+H]⁺), 438.1023.

### EXAMPLE 381

### 5-[(4-bromophenyl)sulfonyl]-2-methyl-N-(2-pyridin-2-ylethyl)benzenesulfonamide

In an analogous manner to Example 375:

Step 3: 2-(2-aminoethyl)-pyridine was used to prepare 5-[4-bromophenyl)sulfonyl-2-methyl-N-(2-pyridin-2-ylethyl)benzenesulfonamide.

MS (ESI+) m/z 495;

HRMS: calcd for C₂₀H₁₉BrN₂O₄S₂+H+, 495.00424; found (ESI, [M+H]⁺), 495.0048.

### EXAMPLE 382

### 2-methyl-5-(phenylsulfonyl)-N-(tetrahydro-2H-pyran-4-ylmethyl)benzenesulfonamide

In an analogous manner to Example 298:

Step 2: 5-Benzenesulfonyl-2-methyl-benzenesulfonyl chloride and 2-(tetrahydro-pyran-4-yl)-methylamine were used to prepare 2-methyl-5-(phenylsulfonyl)-N-(tetrahydro-2H-pyran-4-ylmethyl)benzenesulfonamide.

MS (ESI+) m/z 410;

MS (ESI−) m/z 408;

HRMS: calcd for C₁₉H₂₃NO₅S₂+H+, 410.10904; found (ESI, [M+H]⁺), 410.111.

### EXAMPLE 383

### 5-[(4-fluorophenyl)sulfonyl]-2-methyl-N-(tetrahydro-2H-pyran-4-ylmethyl)benzenesulfonamide

In an analogous manner to Example 298:

Step 2: 2-Methyl-5-(4-fluoro-benzenesulfonyl)-benzenesulfonyl chloride and tetrahydro-pyran-4-yl-methylamine were used to prepare 5-[(4-fluorophenyl)sulfonyl]-2-methyl-N-(tetrahydro-2H-pyran-4-ylmethyl)benzenesulfonamide.

MS (ESI+) m/z 428;

MS (ESI−) m/z 426;

HRMS: calcd for C₁₉H₂₂FNO₅S₂+H+, 428.09962; found (ESI, [M+H]⁺), 428.1015.

### EXAMPLE 384

### 2-ethyl-5-[(4-fluorophenyl)sulfonyl]-N-(tetrahydro-2H-pyran-4-ylmethyl)benzenesulfonamide

In an analogous manner to Example 298:

Step 2: 2-Ethyl-5-(4-fluoro-benzenesulfonyl)-benzenesulfonyl chloride and tetrahydro-pyran-4-yl-methylamine were used to prepare 2-ethyl-5-[(4-fluorophenyl)sulfonyl]-N-(tetrahydro-2H-pyran-4-ylmethyl)benzenesulfonamide.

MS (ESI+) m/z 442;

MS (ESI−) m/z 440;

HRMS: calcd for C₂₀H₂₄FNO₅S₂+H+, 442.11527; found (ESI, [M+H]⁺), 442.1144.

### EXAMPLE 385

### 2,4-dimethyl-5-(phenylsulfonyl)-N-(tetrahydro-2H-pyran-4-ylmethyl)benzenesulfonamide

In an analogous manner to Example 298:

Step 2: 5-Benzenesulfonyl-2,4-dimethyl-benzenesulfonyl chloride and tetrahydro-pyran-4-yl-mthylamine were used to prepare 2,4-dimethyl-5-(phenylsulfonyl)-N-(tetrahydro-2H-pyran-4-ylmethyl)benzenesulfonamide.

MS (ESI+) m/z 424;

MS (ESI−) m/z 422;

HRMS: calcd for C₂₀H₂₅NO₅S₂+H+, 424.12469; found (ESI, [M+H]⁺), 424.124.

### EXAMPLE 386

### N-(2-hydroxy-2-phenylethyl)-2-methyl-5-(phenylsulfonyl)benzenesulfonamide

In an analogous manner to Example 298:

Step 2: 5-Benzenesulfonyl-2-methyl-benzenesulfonyl chloride and 2-amino-1-phenyl-ethanol were used to prepare N-(2-hydroxy-2-phenylethyl)-2-methyl-5-(phenylsulfonyl)benzenesulfonamide.

MS (ESI−) m/z 430;

HRMS: calcd for C₂₁H₂₁NO₅S₂+NH₄⁺, 449.11994; found (ESI, [M+NH4]⁺), 449.119.

### EXAMPLE 387

### 5-[(4-fluorophenyl)sulfonyl]-N-(2-hydroxy-2-phenylethyl)-2-methylbenzenesulfonamide

In an analogous manner to Example 298:

Step 2: 2-Methyl-5-(4-fluoro-benzenesulfonyl)-benzenesulfonyl chloride and 2-amino-1-phenyl-ethanol were used to prepare 5-[(4-fluorophenyl)sulfonyl]-N-(2-hydroxy-2-phenylethyl)-2-methylbenzenesulfonamide.

MS (ESI−) m/z 448;

HRMS: calcd for C₂₁H₂₀FNO₅S₂+NH₄⁺, 467.11052; found (ESI, [M+NH4]⁺), 467.1105.

### EXAMPLE 388

### 2-ethyl-5-[(4-fluorophenyl)sulfonyl]-N-(2-hydroxy-2-phenylethyl)benzenesulfonamide

In an analogous manner to Example 298:

Step 2: 2-Ethyl-5-(4-fluoro-benzenesulfonyl)-benzenesulfonyl chloride and 2-amino-1-phenyl-ethanol were used to prepare 2-ethyl-5-[(4-fluorophenyl)sulfonyl]-N-(2-hydroxy-2-phenylethyl)benzenesulfonamide.

MS (ESI−) m/z 462.

### EXAMPLE 389

### N-(2-hydroxy-2-phenylethyl)-2,4-dimethyl-5-(phenylsulfonyl)benzenesulfonamide

In an analogous manner to Example 298:

Step 2: 5-Benzenesulfonyl-2,4-dimethyl-benzenesulfonyl chloride and 2-amino-1-phenyl-ethanol were used to prepare N-(2-hydroxy-2-phenylethyl)-2,4-dimethyl-5-(phenylsulfonyl)benzenesulfonamide.

MS (ESI−) m/z 444;

HRMS: calcd for C₂₂H₂₃NO₅S₂+NH₄⁺, 463.13559; found (ESI, [M+NH4]⁺), 463.1356.

### EXAMPLE 390

### trans-N-(2-hydroxy-1-methyl-2-phenylethyl)-2-methyl-5-(phenylsulfonyl)benzenesulfonamide

In an analogous manner to Example 298:

Step 2: 5-Benzenesulfonyl-2-methyl-benzenesulfonyl chloride and norephedrine hydrochloride were used to prepare trans-N-(2-hydroxy-1-methyl-2-phenylethyl)-2-methyl-5-(phenylsulfonyl)benzenesulfonamide.

MS (ESI−) m/z 444;

HRMS: calcd for C₂₂H₂₃NO₅S₂+NH₄⁺, 463.13559; found (ESI, [M+NH4]⁺), 463.1376.

### EXAMPLE 391

### 5-[(4-fluorophenyl)sulfonyl]-N-[trans-2-hydroxy-1-methyl-2-phenylethyl]-2-methylbenzenesulfonamide

In an analogous manner to Example 298:

Step 2: 2-Methyl-5-(4-fluoro-benzenesulfonyl)-benzenesulfonyl chloride and norephedrine hydrochloride were used to prepare 5-[(4-fluorophenyl)sulfonyl]-N-[trans-2-hydroxy-1-methyl-2-phenylethyl]-2-methylbenzenesulfonamide.

MS (ESI−) m/z 462;

HRMS: calcd for C₂₂H₂₂FNO₅S₂+NH₄⁺, 481.12617; found (ESI, [M+NH4]⁺), 481.1258.

### EXAMPLE 392

### 2-ethyl-5-[(4-fluorophenyl)sulfonyl]-N-[(trans)-2-hydroxy-1-methyl-2-phenylethyl]benzenesulfonamide

In an analogous manner to Example 298:

Step 2: 2-Ethyl-5-(4-fluoro-benzenesulfonyl)-benzenesulfonyl chloride and norephedrine hydrochloride were used to prepare 2-ethyl-5-[(4-fluorophenyl)sulfonyl]-N-[(trans)-2-hydroxy-1-methyl-2-phenylethyl]benzenesulfonamide.

MS (ESI−) m/z 476;

HRMS: calcd for C₂₃H₂₄FNO₅S₂+NH₄⁺, 495.14182; found (ESI, [M+NH4]⁺), 495.1408.

### EXAMPLE 393

### N-[(trans)-2-hydroxy-1-methyl-2-phenylethyl]-2,4-dimethyl-5-(phenylsulfonyl)benzenesulfonamide

In an analogous manner to Example 298:

Step 2: 5-Benzenesulfonyl-2,4-dimethyl-benzenesulfonyl chloride and norephedrine hydrochloride were used to prepare N-[(trans)-2-hydroxy-1-methyl-2-phenylethyl]-2,4-dimethyl-5-(phenylsulfonyl)benzenesulfonamide.

HRMS: calcd for C₂₃H₂₅NO₅S₂+NH₄⁺, 477.15124; found (ESI, [M+NH4]⁺), 477.1517.

### EXAMPLE 394

### 2-methyl-5-(phenylsulfonyl)-N-propylbenzenesulfonamide

In an analogous manner to Example 294, 5-benzenesulfonyl-2-methyl-benzenesulfonyl chloride, triethylamine, and propylamine hydrochloride in dichloromethane were used to prepare 2-methyl-5-(phenylsulfonyl)-N-propylbenzenesulfonamide.

MS (ES−) m/z 352.0.

### EXAMPLE 395

### N-(tert-butyl)-2-methyl-5-(phenylsulfonyl)benzenesulfonamide

In an analogous manner to Example 294, 5-benzenesulfonyl-2-methyl-benzenesulfonyl chloride, triethylamine, and tert-butylamine in dichloromethane were used to prepare N-(tert-butyl)-2-methyl-5-(phenylsulfonyl)benzenesulfonamide.

MS (ESI−) m/z 366.

### EXAMPLE 396

### N-(2,2-dimethylpropyl)-2-methyl-5-(phenylsulfonyl)benzenesulfonamide

In an analogous manner to Example 294, 5-benzenesulfonyl-2-methyl-benzenesulfonyl chloride, triethylamine, and neo-pentylamine in dichloromethane were used to prepare N-(2,2-dimethylpropyl)-2-methyl-5-(phenylsulfonyl)benzenesulfonamide.

MS (ES−) m/z 380.0.

### EXAMPLE 397

### N-(1-ethylpropyl)-2-methyl-5-(phenylsulfonyl)benzenesulfonamide

In an analogous manner to Example 294, 5-benzenesulfonyl-2-methyl-benzenesulfonyl chloride, triethylamine, and 3-aminopentane in dichloromethane were used to prepare N-(1-ethylpropyl)-2-methyl-5-(phenylsulfonyl)benzenesulfonamide.

MS (ES−) m/z 380.0.

### EXAMPLE 398

### N-cyclobutyl-2-methyl-5-(phenylsulfonyl)benzenesulfonamide

In an analogous manner to Example 294, 5-benzenesulfonyl-2-methyl-benzenesulfonyl chloride, triethylamine, and cyclobutylamine in dichloromethane were used to prepare N-cyclobutyl-2-methyl-5-(phenylsulfonyl)benzenesulfonamide.

MS (ESI−) m/z 364.

### EXAMPLE 399

### N-cyclopentyl-2-methyl-5-(phenylsulfonyl)benzenesulfonamide

In an analogous manner to Example 294, 5-benzenesulfonyl-2-methyl-benzenesulfonyl chloride, triethylamine, and cyclopentylamine in dichloromethane were used to prepare N-cyclopentyl-2-methyl-5-(phenylsulfonyl)benzenesulfonamide.

MS (ESI−) m/z 378.

### EXAMPLE 400

### N-cyclohexyl-2-methyl-5-(phenylsulfonyl)benzenesulfonamide

In an analogous manner to Example 294, 5-benzenesulfonyl-2-methyl-benzenesulfonyl chloride, triethylamine, and cyclohexylamine in dichloromethane were used to prepare N-cyclohexyl-2-methyl-5-(phenylsulfonyl)benzenesulfonamide.

MS (ESI−) m/z 392.

### EXAMPLE 401

### 2-methyl-5-(phenylsulfonyl)-N-(2,2,2-trifluoroethyl)benzenesulfonamide

In an analogous manner to Example 294, 5-benzenesulfonyl-2-methyl-benzenesulfonyl chloride, triethylamine, and 2,2,2-trifluoroethylamine in dichloromethane were used to prepare 2-methyl-5-(phenylsulfonyl)-N-(2,2,2-trifluoroethyl)benzenesulfonamide.

MS (ES−) m/z 391.9.

### EXAMPLE 402

### 5-({4-[(1E)-N-hydroxyethanimidoyl]phenyl}sulfonyl)-2-methyl-N-(tetrahydro-2H-pyran-4-yl)benzenesulfonamide

To a stirred mixture of 5-[(4-acetylphenyl)sulfonyl]-2-methyl-N-(tetrahydro-2H-pyran-4-yl)benzenesulfonamide (0.20 g, 0.46 mmol) in ethanol (2 mL) and D.I. water (0.5 mL) was added sodium acetate (0.05 g, 0.55 mmol) and hydroxylamine hydrochloride (0.04 g, 0.50 mmol). The resulting mixture was heated to reflux for 2.5 hours. The solution was concentrated and filtered to give 5-({4-[(1E)-N-hydroxyethanimidoyl]phenyl}sulfonyl)-2-methyl-N-(tetrahydro-2H-pyran-4-yl)benzenesulfonamide (0.11 g, 55%).

MS (ESI+) m/z 453;

HRMS: calcd for C₂₀H₂₄N₂O₆S₂, 452.10758; found (ESI, [H+M]⁺), 453.1131.

### EXAMPLE 403

### 5-[(4-acetylphenyl)sulfonyl]-2-methyl-N-(2-pyridin-2-ylethyl)benzenesulfonamide

In an analogous manner to Example 380, 5-[(4-bromophenyl)sulfonyl]-2-methyl-N-(2-pyridin-2-ylethyl)benzenesulfonamide was used to prepare 5-[(4-acetylphenyl)sulfonyl]-2-methyl-N-(2-pyridin-2-ylethyl)benzenesulfonamide.

MS (ESI+) m/z 459;

HRMS: calcd for C₂₂H₂₂N₂O₅S₂+H+, 459.10429; found (ESI, [M+H]⁺), 459.1054.

### EXAMPLE 404

### N-(2-hydroxy-1,1-dimethylethyl)-2-methyl-5-(phenylsulfonyl)benzenesulfonamide

In an analogous manner to Example 294, 5-benzenesulfonyl-2-methyl-benzenesulfonyl chloride, triethylamine, and 2-amino-2-methyl-1-propanol in dichloromethane were used to prepare N-(2-hydroxy-1,1-dimethylethyl)-2-methyl-5-(phenylsulfonyl)benzenesulfonamide.

MS (ESI−) m/z 382.

### EXAMPLE 405

### 5-{[4-(1-hydroxy-1-methylethyl)phenyl]sulfonyl}-2-methyl-N-(2-pyridin-2-ylethyl)benzenesulfonamide

To a stirred mixture of 5-[(4-acetylphenyl)sulfonyl]-2-methyl-N-(2-pyridin-2-ylethyl)benzenesulfonamide (0.15 g, 0.33 mmol) in THF (3 mL) was added methyl magnesium bromide 1.4M in 75% toluene/THF (0.75 mL, 1.0 mmol) at 0° C. The resulting mixture was stirred 2 hours, quenched with a saturated aqueous ammonium chloride solution, and extracted several times with ethyl acetate. The combined organic layers were washed with brine, dried over magnesium sulfate, and concentrated. HPLC separation gave 5-{[4-(1-hydroxy-1-methylethyl)phenyl]sulfonyl)-2-methyl-N-(2-pyridin-2-ylethyl)benzenesulfonamide (0.02 g, 13%).

MS (ES+) m/z 475;

HRMS: calcd for C₂₃H₂₆N₂O₅S₂, 474.12831; found (ESI, [H+M]⁺), 475.1382.

### EXAMPLE 406

### 5-{[4-(1-cyclohexyl-1-hydroxyethyl)phenyl]sulfonyl}-2-methyl-N-(2-pyridin-2-ylethyl)benzenesulfonamid

In an analogous manner to Example 405, cyclohexyl magnesium chloride was used to prepare 5-{[4-(1-cyclohexyl-1-hydroxyethyl)phenyl]sulfonyl}-2-methyl-N-(2-pyridin-2-ylethyl)benzenesulfonamide.

MS (ES−) m/z 541.0;

HRMS: calcd for C₂₈H₃₄N₂O₅S₂+H+, 543.19819; found (ESI, [M+H]⁺), 543.1959.

### EXAMPLE 407

### 5-{[4-(1-hydroxy-1-phenylethyl)phenyl]sulfonyl}-2-methyl-N-(2-pyridin-2-ylethyl)benzenesulfonamide

In an analogous manner to Example 405, phenyl magnesium bromide was used to prepare 5-{[4-(1-hydroxy-1-phenylethyl)phenyl]sulfonyl}-2-methyl-N-(2-pyridin-2-ylethyl)benzenesulfonamide.

MS (ES−) m/z 535.0;

HRMS: calcd for C₂₈H₂₈N₂O₅S₂+H+, 537.15124; found (ESI, [M+H]⁺), 537.1492.

### EXAMPLE 408

### 5-{[4-(1-hydroxy-1-methyl-2-phenylethyl)phenyl]sulfonyl}-2-methyl-N-(2-pyridin-2-ylethyl)benzenesulfonamide

In an analogous manner to Example 405, benzyl magnesium chloride was used to prepare 5-{[4-(1-hydroxy-1-methyl-2-phenylethyl)phenyl]sulfonyl}-2-methyl-N-(2-pyridin-2-ylethyl)benzenesulfonamide.

MS (ES−) m/z 549.0;

HRMS: calcd for C₂₉H₃₀N₂O₅S₂+H+, 551.16689; found (ESI, [M+H]⁺), 551.1655.

### EXAMPLE 409

### 5-[(3-cyanophenyl)sulfonyl]-2-methyl-N-(2-pyridin-2-ylethyl)benzenesulfonamide

Step 1: Following the same procedure described in Example 315 (step 1), 2-(2-aminoethyl)-pyridine was used to prepare 5-bromo-2-methyl-N-(2-pyridin-2-ylethyl)benzenesulfonamide.

MS (ES) m/z 352.9;

Step 2: To a stirred solution of 5-bromo-2-methyl-N-(2-pyridin-2-ylethyl)benzenesulfonamide (10.0 g, 28.15 mmol) in THF (200 mL) at −78° C. was added methyl magnesium bromide 1.4M in 75% toluene/THF (24.0 mL, 33.6 mmol). The reaction was stirred 10 minutes and n-butyl lithium 2.5M in hexane (14.0 mL, 35.0 mmol) was added dropwise. The reaction was stirred an additional 10 minutes. Sulfur dioxide was bubbled into the reaction for 20 minutes and was then allowed to warm to room temperature over a period of 2 hours. The reaction was quenched with D.I. water (200 mL) and extracted with ethyl acetate. Sodium hydroxide 2.5M was added to the aqueous layer to a pH of 11 and extracted again with ethyl acetate. The aqueous layer was concentrated and the resulting solid was triturated with methanol to give 4-methyl-3-{[(2-pyridin-2-ylethyl)amino]sulfonyl }benzenesulfinic acid (10.6 g, 103% contains undetermined quantity of inorganic salts.)

MS (ESI) m/z 341;

Step 3: To a stirred solution of 4-methyl-3-{[(2-pyridin-2ylethyl)amino]sulfonyl}benzenesulfinic acid (0.30 g, 0.83 mmol) in DMSO (4 mL) was added 3-cyanophenylboronic acid (0.12 g, 0.83 mmol), potassium carbonate (0.46 g, 3.31 mmol), and copper (II) acetate (0.16 g, 0.91 mmol). The resulting solution was stirred overnight at room temperature under a drying tube. The reaction was partitioned between ammonium chloride solution (sat) and ethyl acetate. The organic layer was concentrated. HPLC separation gave 5-[(3-cyanophenyl)sulfonyl]-2-methyl-N-(2-pyridin-2-ylethyl)benzenesulfonamide (0.02 g, 5%).

MS (ES+) m/z 442.0;

MS (ES−) m/z 440.0.

### EXAMPLE 410

### 2-methyl-5-(1-naphthylsulfonyl)-N-(2-pyridin-2-ylethyl)benzenesulfonamide

In an analogous manner to Example 409,

Step 3: 1-naphthylene boronic acid was used to prepare 2-methyl-5-(1-naphthylsulfonyl)-N-(2-pyridin-2-ylethyl)benzenesulfonamide.

MS (ES−) m/z 465.0;

HRMS: calcd for C₂₄H₂₂N₂O₄S₂+H+, 467.10937; found (ESI, [M+H]⁺), 467.1113.

### EXAMPLE 411

### 2-methyl-5-(phenylsulfonyl)-N-(2,2,6,6-tetramethylpiperidin-4-yl)benzenesulfonamide

In an analogous manner to Example 294, 5-benzenesulfonyl-2-methyl-benzenesulfonyl chloride, triethylamine, and 4-amino-2,2,4,4-tetramethylpiperidine in dichloromethane were used to prepare 2-methyl-5-(phenylsulfonyl)-N-(2,2,6,6-tetramethylpiperidin-4-yl)benzenesulfonamide.

MS (ESI+) m/z 451;

MS (ESI−) m/z 449.

### EXAMPLE 412

### N-[(1S*,2S*)-2-hydroxycyclohexyl]-2-methyl-5-(phenylsulfonyl)benzenesulfonamide

In an analogous manner to Example 294, 5-benzenesulfonyl-2-methyl-benzenesulfonyl chloride, triethylamine, and trans-2-aminocyclohexanol hydrochloride in dichloromethane were used to prepare N-[(1S*,2S*)-2-hydroxycyclohexyl]-2-methyl-5-(phenylsulfonyl)benzenesulfonamide.

MS (ESI+) m/z 410;

MS (ESI−) m/z 408.

### EXAMPLE 413

### N-(1-benzylpiperidin-4-yl)-2-methyl-5-(phenylsulfonyl)benzenesulfonamide

In an analogous manner to Example 294, 5-benzenesulfonyl-2-methyl-benzenesulfonyl chloride, triethylamine, and 4-amino-1-benzylpiperidine in dichloromethane were used to prepare N-(1-benzylpiperidin-4-yl)-2-methyl-5-(phenylsulfonyl)benzenesulfonamide.

MS (ESI+) m/z 485;

MS (ESI−) m/z 483.

### EXAMPLE 414

### N-(1-benzylpyrrolidin-3-yl)-2-methyl-5-(phenylsulfonyl)benzenesulfonamide

In an analogous manner to Example 294, 5-benzenesulfonyl-2-methyl-benzenesulfonyl chloride, triethylamine, and 1-benzyl-3-aminopyrrolidine in dichloromethane were used to prepare N-(1-benzylpyrrolidin-3-yl)-2-methyl-5-(phenylsulfonyl)benzenesulfonamide.

MS (ESI+) m/z 471;

MS (ESI−) m/z 469.

### EXAMPLE 415

### N-(2,3-dihydro-1H-inden-1-yl)-2-methyl-5-(phenylsulfonyl)benzenesulfonamide

In an analogous manner to Example 294, 5-benzenesulfonyl-2-methyl-benzenesulfonyl chloride, triethylamine, and 1-aminoindane in dichloromethane were used to prepare N-(2,3-dihydro-1H-inden-1-yl)-2-methyl-5-(phenylsulfonyl)benzenesulfonamide.

MS (ESI−) m/z 426.

### EXAMPLE 416

### 5-[(3-hydroxyphenyl)sulfonyl]-2-methyl-N-(2-pyridin-2-ylethyl)benzenesulfonamide

In an analogous manner to Example 409,

Step 3:3-hydroxyphenyl boronic acid was used to prepare 5-(3 hydroxyphenyl)sulfonyl]-2-methyl-N-(2-pyridin-2-ylethyl)benzenesulfonamide.

HRMS: calcd for C₂₀H₂₀N₂O₅S₂+H+, 433.08864; found (ESI, [M+H]⁺), 433.0912.

### EXAMPLE 417

### 5-[(3,5-difluorophenyl)sulfonyl]-2-methyl-N-(2-pyridin-2-ylethyl)benzenesulfonamide

In an analogous manner to Example 409,

Step 3: 3,5-difluorophenyl boronic acid was used to prepare 5-[(3,5-difluorophenyl)sulfonyl]-2-methyl-N-(2-pyridin-2-ylethyl)benzenesulfonamide.

MS (ES−) m/z 451.0;

HRMS: calcd for C₂₀H₁₈F₂N₂O₄S₂+H+, 453.07488; found (ESI, [M+H]⁺), 453.0736.

### EXAMPLE 418

### 5-[(4-ethylphenyl)sulfonyl]-2-methyl-N-(2-pyridin-2-ylethyl)benzenesulfonamide

In an analogous manner to Example 409,

Step 3: 4-ethylphenyl boronic acid was used to prepare 5-[(4-ethylphenyl)sulfonyl]-2-methyl-N-(2-pyridin-2-ylethyl)benzenesulfonamide.

MS (ES−) m/z 443.0;

HRMS: calcd for C₂₂H₂₄N₂O₄S₂+H+, 445.12502; found (ESI, [M+H]⁺), 445.1262.

### EXAMPLE 419

### 5-[(3-acetylphenyl)sulfonyl]-2-methyl-N-(2-pyridin-2-ylethyl)benzenesulfonamide

In an analogous manner to Example 409,

Step 3: 2-acetylphenyl boronic acid was used to prepare 5-[(3-acetylphenyl)sulfonyl]-2-methyl-N-(2-pyridin-2-ylethyl)benzenesulfonamide.

MS (ES−) m/z 457.0;

HRMS: calcd for C₂₂H₂₂N₂O₅S₂+H+, 459.10429; found (ESI, [M+H]⁺), 459.1059.

### EXAMPLE 420

### 5-[(2-ethoxyphenyl)sulfonyl]-2-methyl-N-(2-pyridin-2-ylethyl)benzenesulfonamide

In an analogous manner to Example 409,

Step 3: 2-ethyoxyphenyl boronic acid was used to prepare 5-[(2-ethoxyphenyl)sulfonyl]-2-methyl-N-(2-pyridin-2-ylethyl)benzenesulfonamide.

MS (ES−) m/z 459.0;

HRMS: calcd for C₂₂H₂₄N₂O₅S₂+H+, 461.11994; found (ESI, [M+H]⁺), 461.1215.

### EXAMPLE 421

### 5-[(2,5-dimethoxyphenyl)sulfonyl]-2-methyl-N-(2-pyridin-2-ylethyl)benzenesulfonamide

In an analogous manner to Example 409,

Step 3: 2,5-dimethoxyphenyl boronic acid was used to prepare 5-[(2,5-dimethoxyphenyl)sulfonyl]-2-methyl-N-(2-pyridin-2-ylethyl)benzenesulfonamide.

MS (ES−) m/z 475.0;

HRMS: calcd for C₂₂H₂₄N₂O₆S₂+H+, 477.11485; found (ESI, [M+H]⁺), 477.1174.

### EXAMPLE 422

### 5-[(2,3-dimethoxyphenyl)sulfonyl]-2-methyl-N-(2-pyridin-2-ylethyl)benzenesulfonamide

In an analogous manner to Example 409,

Step 3: 2,3-dimethoxyphenyl boronic acid was used to prepare 5-[(2,3-dimethoxyphenyl)sulfonyl]-2-methyl-N-(2-pyridin-2-ylethyl)benzenesulfonamide.

MS (ES−) m/z 475.0;

HRMS: calcd for C₂₂H₂₄N₂O₆S₂+H+, 477.11485; found (ESI, [M+H]⁺), 477.1169.

### EXAMPLE 423

### 5-[(2,4-dimethoxyphenyl)sulfonyl]-2-methyl-N-(2-pyridin-2-ylethyl)benzenesulfonamide

In an analogous manner to Example 409,

Step 3: 2,4-dimethoxyphenyl boronic acid was used to prepare 5-[(2,4-dimethoxyphenyl)sulfonyl]-2-methyl-N-(2-pyridin-2-ylethyl)benzenesulfonamide.

MS (ES−) m/z 475.0;

HRMS: calcd for C₂₂H₂₄N₂O₆S₂+H+, 477.11485; found (ESI, [M+H]⁺), 477.1144.

### EXAMPLE 424

### N-cyclopropyl-2-methyl-5-(phenylsulfonyl)benzenesulfonamide

In an analogous manner to Example 294, 5-benzenesulfonyl-2-methyl-benzenesulfonyl chloride, triethylamine, and cyclopropylamine in dichloromethane were used to prepare N-cyclopropyl-2-methyl-5-(phenylsulfonyl)benzenesulfonamide.

MS (ES−) m/z 350.0.

### EXAMPLE 425

### ethyl 4-({[2-methyl-5-(phenylsulfonyl)phenyl]sulfonyl}amino)piperidine-1-carboxylate

In an analogous manner to Example 294, 5-benzenesulfonyl-2-methyl-benzenesulfonyl chloride, triethylamine, and ethyl 4-amino-1-piperidine carboxylate in dichloromethane were used to prepare ethyl 4-({[2-methyl-5-(phenylsulfonyl)phenyl]sulfonyl}amino)piperidine-1-carboxylate.

MS (ES−) m/z 465.0.

### EXAMPLE 426

### 2-methyl-N-(2-pyridin-2-ylethyl)-5-(pyridin-3-ylsulfonyl)benzenesulfonamide

In an analogous manner to Example 409,

Step 3: pyridine-3-boronic acid was used to prepare 2-methyl-N-(2-pyridin-2-ylethyl)-5-(pyridin-3-ylsulfonyl)benzenesulfonamide.

MS (ESI+) m/z 418.

### EXAMPLE 427

### 5-(1H-indol-5-ylsulfonyl)-2-methyl-N-(2-pyridin-2-ylethyl)benzenesulfonamide

In an analogous manner to Example 409,

Step 3: 5-indole boronic acid was used to prepare 5-(1H-indol-5-ylsulfonyl)-2-methyl-N-(2-pyridin-2-ylethyl)benzenesulfonamide.

MS (ES+) m/z 456.1.

### EXAMPLE 428

### 5-[(4-cyanophenyl)sulfonyl]-2-methyl-N-(2-pyridin-2-ylethyl)benzenesulfonamide

In an analogous manner to Example 409,

Step 3: 4-cyanophenyl boronic acid was used to prepare 5-[(4-cyanophenyl)sulfonyl]-2-methyl-N-(2-pyridin-2-ylethyl)benzenesulfonamide.

MS (ESI+) m/z 442.

### EXAMPLE 429

### 5-{[3-(ethylsulfonyl)phenyl]sulfonyl}-2-methyl-N-(2-pyridin-2-ylethyl)benzenesulfonamide

In an analogous manner to Example 409,

Step 3: 3-(ethylsulfonyl)phenyl boronic acid was used to prepare 5-{[3-(ethylsulfonyl)phenyl]sulfonyl}-2-methyl-N-(2-pyridin-2-ylethyl)benzenesulfonamide.

MS (ES+) m/z 509.0.

### EXAMPLE 430

### 2-methyl-5-[(2-methylphenyl)sulfonyl]-N-(2-pyridin-2-ylethyl)benzenesulfonamide

In an analogous manner to Example 409,

Step 3: 2-methylphenyl boronic acid was used to prepare 2-methyl-5-[(2-methylphenyl)sulfonyl]-N-(2-pyridin-2-ylethyl)benzenesulfonamide.

MS (ESI+) m/z 431.

### EXAMPLE 431

### 5-[(2-ethylphenyl)sulfonyl]-2-methyl-N-(2-pyridin-2-ylethyl)benzenesulfonamide

In an analogous manner to Example 409,

Step 3: 2-ethylphenyl boronic acid was used to prepare 5-[(2-ethylphenyl)sulfonyl]-2-methyl-N-(2-pyridin-2-ylethyl)benzenesulfonamide.

MS (ESI+) m/z 445.

### EXAMPLE 432

### 5-(biphenyl-2-ylsulfonyl)-2-methyl-N-(2-pyridin-2-ylethyl)benzenesulfonamide

In an analogous manner to Example 409,

Step 3: 2-biphenyl boronic acid was used to prepare 5-(biphenyl-2-ylsulfonyl)-2-methyl-N-(2-pyridin-2-ylethyl)benzenesulfonamide.

MS (ESI+) m/z 493.

### EXAMPLE 433

### 5-(biphenyl-4-ylsulfonyl)-2-methyl-N-(2-pyridin-2-ylethyl)benzenesulfonamide

In an analogous manner to Example 409,

Step 3: 4-biphenyl boronic acid was used to prepare 5-(biphenyl-4-ylsulfonyl)-2-methyl-N-(2-pyridin-2-ylethyl)benzenesulfonamide.

MS (ESI+) m/z 493.

### EXAMPLE 434

### 5-(biphenyl-3-ylsulfonyl)-2-methyl-N-(2-pyridin-2-ylethyl)benzenesulfonamide

In an analogous manner to Example 409,

Step 3: 3-biphenyl boronic acid was used to prepare 5-(biphenyl-3-ylsulfonyl)-2-methyl-N-(2-pyridin-2-ylethyl)benzenesulfonamide.

MS (ESI+) m/z 493.

### EXAMPLE 435

### 5-[(4-tert-butylphenyl)sulfonyl]-2-isopropyl-N-(2-pyridin-2-ylethyl)benzenesulfonamide

In an analogous manner to Example 230,

Step 1: 4-Isopropylbenzenesulfonyl chloride and tert-butylbenzene were used to prepare 1-tert-butyl-4-[(4-isopropylphenyl)sulfonyl]benzene.

Step 2: 1-tert-Butyl-4-[(4-isopropylphenyl)sulfonyl]benzene and chlorosulfonic acid were used to prepare 5-(4-tert-butyl-benzenesulfonyl)-2-isopropyl-benzenesulfonyl chloride.

Step 3: To a solution of 5-(4-tert-Butyl-benzenesulfonyl)-2-isopropyl-benzenesulfonyl chloride (150 mg, 0.36 mmol) in dichloromethane (4.0 mL) was added pyridine (73 mg, 0.72 mmol) and 2-(2-aminoethyl)pyridine (44 mg, 0.36 mmol). The reaction was stirred at room temperature overnight and then extracted with saturated sodium bicarbonate (2×10 mL), followed by saturated ammonium chloride (2×10 mL). The organic layer was dried with magnesium sulfate and concentrated down under vacuum. The crude residue was purified using automated flash chromatography with a gradient mobile phase consisting of ethyl acetate and hexane resulting in the isolation of 5-[(4-tert-butylphenyl)sulfonyl]-2-isopropyl-N-(2-pyridin-2-ylethyl)benzenesulfonamide (178 mg, 98%).

MS (ES+) m/z 501.

### EXAMPLE 436

### 5-[(4-tert-butylphenyl)sulfonyl]-N-[3-(1H-imidazol-1-yl)propyl]-2-isopropylbenzenesulfonamide

In an analogous manner to Example 435, 5-(4-tert-Butyl-benzenesulfonyl)-2-isopropyl-benzenesulfonyl chloride and 1-(3-aminopropyl)imidazole were used to prepare 5-[(4-tert-butylphenyl)sulfonyl]-N-[3-(1H-imidazol-1-yl)propyl]-2-isopropylbenzenesulfonamide.

MS (ES+) m/z 504;

HRMS: calcd. for C₂₅H₃₃N₃O₄S₂+H+, 504.1991: found (ESI, [m+H]⁺), 504.2007.

### EXAMPLE 437

### 5-[(4-tert-butylphenyl)sulfonyl]-2-isopropyl-N-(tetrahydro-2H-pyran-4-yl)benzenesulfonamide

In an analogous manner to Example 435, 5-(4-tert-Butyl-benzenesulfonyl)-2-isopropyl-benzenesulfonyl chloride and 4-aminotetrahydropyran were used to prepare 5-[(4-tert-butylphenyl)sulfonyl]-2-isopropyl-N-(tetrahydro-2H-pyran-4-yl)benzenesulfonamide.

MS (ES+) m/z 480.

### EXAMPLE 438

### 5-[(4-tert-butylphenyl)sulfonyl]-2-methyl-N-(2-pyridin-2-ylethyl)benzenesulfonamide

In an analogous manner to Example 230,

Step 1: p-Toluenesulfonyl chloride and tert-butylbenzene were used to prepare 1-tert-butyl-4-[(4-methylphenyl)sulfonyl]benzene.

Step 2: 1-tert-Butyl-4-[(4-methylphenyl)sulfonyl]benzene and chlorosulfonic acid were used to prepare 5-(4-tert-butyl-benzenesulfonyl)-2-methyl-benzenesulfonyl chloride.

Step 3: In an analogous manner to Example 435, 5-(4-tert-Butyl-benzenesulfonyl)-2-methyl-benzenesulfonyl chloride and 2-(2-aminoethyl)pyridine were used to prepare 5-[(4-tert-butylphenyl)sulfonyl]-2-methyl-N-(2-pyridin-2-ylethyl)benzenesulfonamide.

MS (ES+) m/z 473;

HRMS: calcd. for C₂₄H₂₈N₂O₄S₂+H⁺, 473.1569; found (ESI, [m+H]⁺), 473.1551.

### EXAMPLE 439

### 5-[(4-tert-butylphenyl)sulfonyl]-N-[3-(1H-imidazol-1-yl)propyl]-2-methylbenzenesulfonamide

In an analogous manner to Example 435, 5-(4-tert-Butyl-benzenesulfonyl)-2-methyl-benzenesulfonyl chloride and 1-(3-aminopropyl)imidazole were used to prepare 5-[(4-tert-butylphenyl)sulfonyl]-N-[3-(1H-imidazol-1-yl)propyl]-2-methylbenzenesulfonamide.

MS (ES+) m/z 476;

HRMS: calcd. for C₂₃H₂₉N₃O₄S₂+H⁺, 476.1678; found (ESI, [m+H]⁺), 476.1662.

### EXAMPLE 440

### 5-[(4-tert-butylphenyl)sulfonyl]-2-methyl-N-(tetrahydro-2H-pyran-4-yl)benzenesulfonamide

In an analogous manner to Example 435, 5-(4-tert-Butyl-benzenesulfonyl)-2-methyl-benzenesulfonyl chloride and 4-aminotetrahydropyran were used to prepare 5-[(4-tert-butylphenyl)sulfonyl]-2-methyl-N-(tetrahydro-2H-pyran-4-yl)benzenesulfonamide.

MS (ES+) m/z 452.

### EXAMPLE 441

### N-[2-(2-Fluorophenyl)ethyl]-3-[(4-methylphenyl)sulfonyl]-5,6,7,8-tetrahydronaphthalene-1-sulfonamide

Step a: A stirred solution of 1,2,3,4-tetrahydronaphthalene (10.0 mL, 74 mmol) and p-toluenesulfonyl chloride (3.81 g, 20 mmol) was cooled to 0° C. and treated slowly under nitrogen with solid anhydrous aluminum chloride (3.20 g, 24 mmol). After stirring neat for 4 hours at room temperature, the mixture was slowly poured into ice water and extracted with ethyl acetate (2×). The organic phase was washed with a saturated, aqueous sodium chloride solution, dried over anhydrous sodium sulfate, and filtered through a short column of silica gel. The filtrate was evaporated in vacuo to yield a crude oil containing a mixture of isomers in approximately a 6:1 ratio. The crude oil was recrystallized twice from diethyl ether-hexane to yield 4-methylphenyl 5,6.7,8-tetrahydronaphthalen-2-yl sulfone (3.04 g, 53%) as a homogeneous, colorless, crystalline solid, m.p. 130-132° C.; MS(EI) m/z 286; HRMS: calcd for C₁₇H₁₈O₂S, 286.10275; found (EI, M+.), 286.1022.

Step b: 4-Methylphenyl 5,6,7,8-tetrahydronaphthalen-2-yl sulfone (0.29 g, 1.0 mmol) was heated with stirring at 60° C. for one hour under nitrogen with chlorosulfonic acid (0.67 mL, 1.17 g, 10.0 mmol). The mixture was cooled to room temperature, poured slowly into a cold solution of 1N hydrochloric acid, and extracted with ethyl acetate (2×.). The organic phase was washed with a saturated, aqueous sodium chloride solution, dried over anhydrous sodium sulfate, filtered, and the solvent concentrated in vacuo to an oil. The oil was dissolved in dichloromethane and treated dropwise under nitrogen with a solution of 2-fluorophenethylamine (0.28 g, 2.0 mmol) in dichloromethane. After stirring for one hour at room temperature, the reaction mixture was concentrated in vacuo and diluted with ethyl acetate. The organic phase was washed sequentially with 1N hydrochloric acid and a saturated, aqueous sodium chloride solution, dried over anhydrous sodium sulfate, filtered through a short column of silica gel, and the filtrate concentrated to a crude oil. The crude oil was purified by preparative liquid chromatography on a Biotage® 40 Mi column of pre-packed silica gel (90 g) eluting with a gradient of 25% -50% methyl tert-butyl ether in hexane at a flow rate of 50 ml/min to afford, after evaporation of the solvent under vacuum, N-[2-(2-fluorophenyl)ethyl]-3-[(4-methylphenyl)sulfonyl]-5.6,7,8-tetrahydronaphthalene-1-sulfonamide (0.12 g, 25%) as a colorless, homogeneous, amorphous foam, m.p. 55-60° C.;

MS (ES−) m/z 486.1;

MS (ES+) m/z 488.1.

### EXAMPLE 442

### 5-[(4-fluorophenyl)sulfonyl]-N-[2-(1H-imidazol-4-yl)ethyl]-2-isopropylbenzenesulfonamide

In an analogous manner to Example 298:

Step 3: 2-Isopropyl-5-(4-fluoro-benzenesulfonyl)-benzenesulfonyl chloride and 2-(1H-imidazol-4-yl)-ethylamine were used to prepare 5-[(4-fluorophenyl)sulfonyl]-N-[2-(1H-imidazol-4-yl)ethyl]-2-isopropylbenzenesulfonamide.

MS (ES+) m/z 452.0;

MS (ES−) m/z 450.0.

### EXAMPLE 443

### 5-[(4-fluorophenyl)sulfonyl]-2-isopropyl-N-[(2R)-2-phenylpropyl]benzenesulfonamide

In an analogous manner to Example 298:

Step 3: 2-Isopropyl-5-(4-fluoro-benzenesulfonyl)-benzenesulfonyl chloride and 2-(R)-phenylpropyl-1-amine were used to prepare 5-[(4-fluorophenyl)sulfonyl]-2-isopropyl-N-[(2R)-2-phenylpropyl]benzenesulfonamide.

MS (ES+) m/z 476.1;

MS (ES−) m/z 474.1.

### EXAMPLE 444

### 5-[(4-fluorophenyl)sulfonyl]-2-isopropyl-N-[(2S)-2-phenylpropyl]benzenesulfonamide

In an analogous manner to Example 298:

Step 3: 2-Isopropyl-5-(4-fluoro-benzenesulfonyl)-benzenesulfonyl chloride and 2-(S)-phenylpropyl-1-amine were used to prepare 5-[(4-fluorophenyl)sulfonyl]-2-isopropyl-N-[(2S)-2-phenylpropyl]benzenesulfonamide.

MS (ES−) m/z 474.1.

### EXAMPLE 445

### 5-[(4-fluorophenyl)sulfonyl]-2-isopropyl-N-(tetrahydro-2H-pyran-4-ylmethyl)benzenesulfonamide

In an analogous manner to Example 298:

Step 3: 2-Isopropyl-5-(4-fluoro-benzenesulfonyl)-benzenesulfonyl chloride and tetrahydro-pyran-4-yl-methylamine were used to prepare 5-[(4-fluorophenyl)sulfonyl]-2-isopropyl-N-(tetrahydro-2H-pyran-4-ylmethyl)benzenesulfonamide.

MS (ES+) m/z 456.1;

MS (ES−) m/z 454.1.

### EXAMPLE 446

### tert-butyl 4-({[2-methyl-5-(phenylsulfonyl)phenyl]sulfonyl}amino)piperidine-1-carboxylate

In an analogous manner to Example 435, 5-benzenesulfonyl-2-methyl-benzenesulfonyl chloride and 4-amino-1-Boc-piperidine were used to prepare tert-butyl 4-({[2-methyl-5-(phenylsulfonyl)phenyl]sulfonyl}amino)piperidine-1-carboxylate.

MS (ES−) m/z 493.

### EXAMPLE 447

### 2-methyl-5-(phenylsulfonyl)-N-piperidin-4-ylbenzenesulfonamide

To a solution of 25% trifluoroacetic acid/dichloromethane was added tert-butyl 4-({[2-methyl-5-(phenylsulfonyl)phenyl]sulfonyl}amino)piperidine-1-carboxylate (2.0 g, 4.04 mmol) and the solution was stirred at room temperature for two hours. The reaction was then extracted with saturated sodium bicarbonate, dried with magnesium sulfate, and concentrated down under vacuum to give 2-methyl-5-(phenylsulfonyl)-N-piperidin-4-ylbenzenesulfonamide (1.5 g, 94%).

MS (ES+) m/z 495

MS (ES−) m/z 493.

### EXAMPLE 448

### 2-methyl-5-(phenylsulfonyl)-N-[1-(phenylsulfonyl)piperidin-4-yl]benzenesulfonamide

To a solution of 2-methyl-5-(phenylsulfonyl)-N-piperidin-4-ylbenzenesulfonamide (125 mg, 0.32 mmol) and pyridine (48 mg, 0.48 mmol) in dichloromethane (4 mL) was added benzenesulfonyl chloride (59 mg, 0.33 mmol). The reaction was stirred at room temperature for three hours and then extracted with saturate sodium bicarbonate. The aqueous phase was washed with dichloromethane (2×10 mL). The combined organic phases were dried with magnesium sulfate and concentrated down under vacuum. The crude residue was purified using automated flash chromatography with a gradient mobile phase consisting of ethyl acetate and hexane resulting in the isolation of 2-methyl-5-(phenylsulfonyl)-N-[1-(phenylsulfonyl)piperidin-4-yl]benzenesulfonamide (95 mg, 56%).

MS (ES+) m/z 535

MS (ES−) m/z 533.

### EXAMPLE 449

### N-[1-(2-furoyl)piperidin-4-yl]-2-methyl-5-(phenylsulfonyl)benzenesulfonamide

To a slurry of 2-methyl-5-(phenylsulfonyl)-N-piperidin-4-ylbenzenesulfonamide (75 mg, 0.19 mmol), morphlinomethyl-polystyrene (170 mg, 62 mmol), and dimethylaminopyridine-polystyrene (70 mg, 0.14 mmol) in dichloromethane (8 mL) was added 2-furoyl chloride (50 mg, 0.38 mmol). The reaction was rotated on an orbital shaker for four hours. Then aminomethylated-polystyrene (150 mg, 0.33 mmol) was added to the reaction and it was rotated for an addition two and one-half hours. The reaction was then filtered and the resin was washed alternately with dichloromethane (3×5 mL) and methanol (3×5 mL) and concentrated down under vacuum. The crude residue was purified using automated flash chromatography with a gradient mobile phase consisting of ethyl acetate and hexane resulting in the isolation of N-[1-(2-furoyl)piperidin-4-yl]-2-methyl-5-(phenylsulfonyl)benzenesulfonamide (68 mg, 73%).

MS (ES+) m/z 489.

### EXAMPLE 450

### 5-({4-[(2-cyanoethyl)amino]phenyl}sulfonyl)-N-[2-(1H-imidazol-1-yl)ethyl]-2-isopropylbenzenesulfonamide

In an analogous manner to Example 353, 5-[(4-fluorophenyl)sulfonyl]-N-[2-(1H-imidazol-1-yl)ethyl]-2-isopropylbenzenesulfonamide and 3-aminopropionitrile were used to prepare 5-({4-[(2-cyanoethyl)amino]phenyl}sulfonyl)-N-[2-(1H-imidazol-1-yl)ethyl]-2-isopropylbenzenesulfonamide.

MS (ESI+) m/z 502;

MS (ESI−) m/z 500.

### EXAMPLE 451

### N-[2-(1H-imidazol-1-yl)ethyl]-2-isopropyl-5-{[4-(methylamino)phenyl]sulfonyl)benzenesulfonamide

In an analogous manner to Example 356, 5-[(4-fluorophenyl)sulfonyl]-N-[2-(1H-imidazol-1-yl)ethyl]-2-isopropylbenzenesulfonamide and methylamine were used to prepare N-[2-(1H-imidazol-1-yl)ethyl]-2-isopropyl-5-{[4-(methylamino)phenyl]sulfonyl}benzenesulfonamide.

MS (ESI+) m/z 463;

MS (ESI−) m/z 461.

### EXAMPLE 452

### 5-({4-[(2-hydroxybutyl)amino]phenyl}sulfonyl)-N-[2-(1H-imidazol-1-yl)ethyl]-2-isopropylbenzenesulfonamide

In an analogous manner to Example 353, 5-[(4-fluorophenyl)sulfonyl]-N-[2-(1H-imidazol-1-yl)ethyl]-2-isopropylbenzenesulfonamide and 2-hydroxybutylamine were used to prepare 5-({4-[(2-hydroxybutyl)amino]phenyl}sulfonyl)-N-[2-(1H-imidazol-1-yl)ethyl]-2-isopropylbenzenesulfonamide.

MS (ESI+) m/z 521;

MS (ESI−) m/z 519.

### EXAMPLE 453

### 5-[(4-{[(2S)-1-(hydroxymethyl)-2-methylbutyl]amino}phenyl)sulfonyl]-N-[2-(1H-imidazol-1-yl)ethyl]-2-isopropylbenzenesulfonamide

In an analogous manner to Example 353, 5-[(4-fluorophenyl)sulfonyl]-N-[2-(1H-imidazol-1-yl)ethyl]-2-isopropylbenzenesulfonamide and L-isoleucinol were used to prepare 5-[(4-{[(2S)-1-(hydroxymethyl)-2-methylbutyl]amino}phenyl)sulfonyl]-N-[2-(1H-imidazol-1-yl)ethyl]-2-isopropylbenzenesulfonamide.

MS (ESI+) m/z 549;

MS (ESI−) m/z 547.

### EXAMPLE 454

### 5-[(3,5-dimethylphenyl)sulfonyl]-2-isopropyl-N-(2-pyridin-2-ylethyl)benzenesulfonamide

Step 1: Following the procedure described in Example 409 (Step 2), 5-bromo-2-isopropyl-N-(2-pyridin-2-ylethyl)benzenesulfonamide (Example 474 Steps 1 and 2) was used to prepare 4-isopropyl-3-{[(2-pyridin-2-ylethyl)amino]sulfonyl}benzenesulfinic acid.

Step 2: Following the procedure described in Example 409 (Step 3), 4-isopropyl-3-{[(2-pyridin-2-ylethyl)amino]sulfonyl}benzenesulfinic acid and 3,5-dimethylphenyl boronic acid were used to prepare 5-[(3,5-dimethylphenyl)sulfonyl]-2-isopropyl-N-(2-pyridin-2-ylethyl)benzenesulfonamide.

MS (ESI+) m/z 473;

MS (ESI−) m/z 471.

### EXAMPLE 455

### 5-[(3-chlorophenyl)sulfonyl]-2-isopropyl-N-(2-pyridin-2-ylethyl)benzenesulfonamide

In an analogous manner to Example 454,

Step 2: 3-chlorophenyl boronic acid was used to prepare 5-[(3-chlorophenyl)sulfonyl]-2-isopropyl-N-(2-pyridin-2-ylethyl)benzenesulfonamide.

MS (ESI+) m/z 479;

MS (ESI−) m/z 477.

### EXAMPLE 456

### 2-isopropyl-5-[(3-methoxyphenyl)sulfonyl]-N-(2-pyridin-2-ylethyl)benzenesulfonamide

In an analogous manner to Example 454,

Step 2: 3-methyoxyphenyl boronic acid was used to prepare 2-isopropyl-5-[(3-methoxyphenyl)sulfonyl]-N-(2-pyridin-2-ylethyl)benzenesulfonamide.

MS (ESI+) m/z 475;

MS (ESI−) m/z 473.

### EXAMPLE 457

### 2-isopropyl-5-[(2-methoxyphenyl)sulfonyl]-N-(2-pyridin-2-ylethyl)benzenesulfonamide

In an analogous manner to Example 454,

Step 2: 2-methoxyphenyl boronic acid was used to prepare 2-isopropyl-5-[(2-methoxyphenyl)sulfonyl]-N-(2-pyridin-2-ylethyl)benzenesulfonamide.

MS (ESI+) m/z;

MS (ESI−) m/z.

### EXAMPLE 458

### 5-[(3,5-difluorophenyl)sulfonyl]-2-isopropyl-N-(2-pyridin-2-ylethyl)benzenesulfonamide

In an analogous manner to Example 454,

Step 2: 3,5-difluorophenyl boronic acid was used to prepare 5-[(3,5-difluorophenyl)sulfonyl]-2-isopropyl-N-(2-pyridin-2-ylethyl)benzenesulfonamide.

MS (ESI+) m/z 481;

MS (ESI−) m/z 479.

### EXAMPLE 459

### 2-cyclohexyl-5-(phenylsulfonyl)-N-(2-pyridin-2-ylethyl)benzenesulfonamide

Step 1: Following the same procedure described in Example 474 (Step 1), 2-cyclohexylbromobenzene was used to prepare 2-cyclohexyl-N-(2-pyridin-2-ylethyl)benzenesulfonamide.

MS (ESI+) m/z 345;

MS (ESI−) m/z 343.

Step 2: Following the same procedure described in Example 474 (Step 2), 2-cyclohexyl-N-(2-pyridin-2-ylethyl)benzenesulfonamide was used to prepare 5-bromo-2-cyclohexyl-N-(2-pyridin-2-ylethyl)benzenesulfonamide.

MS (ESI+) m/z 423;

MS (ESI−) m/z 421.

Step 3: Following the same procedure described in Example 409 (Step 2), 5-bromo-2-cyclohexyl-N-(2-pyridin-2-ylethyl)benzenesulfonamide was used to prepare 4-cyclohexyl-3-{[(2-pyridin-2-ylethyl)amino]sulfonyl}benzenesulfinic acid.

Step 4: Following the same procedure in Example 409 (Step 3), 4-cyclohexyl-3-{[(2-pyridin-2-ylethyl)amino]sulfonyl}benzenesulfinic acid and phenyl boronic acid was used to prepare 2-cyclohexyl-5-(phenylsulfonyl)-N-(2-pyridin-2-ylethyl)benzenesulfonamide

MS (ESI+) m/z 485;

MS (ESI−) m/z 483.

### EXAMPLE 460

### 2-cyclohexyl-5-[(4-fluorophenyl)sulfonyl]-N-(2-pyridin-2-ylethyl)benzenesulfonamide

In an analogous manner to Example 459,

Step 4: 4-fluorophenyl boronic acid was used to prepare 2-cyclohexyl-5-[(4-fluorophenyl)sulfonyl]-N-(2-pyridin-2-ylethyl)benzenesulfonamide.

MS (ESI+) m/z 503;

MS (ESI−) m/z 501.

### EXAMPLE 461

### 2-tert-butyl-5-(phenylsulfonyl)-N-(2-pyridin-2-ylethyl)benzenesulfonamide

Step 1: To a stirred solution of 2-tertbutylbenzenethiol (4.72 g, 28.4 mmol) in concentrated sulfuric acid (90 mL) at 0° C. was added 6% sodium hypochlorite solution (426 mL) dropwise. The resulting mixture was extracted with methylene chloride. Triethylamine (10.0 mL, 71.7 mmol) and 2-(2-aminoethyl)pyridine (4.0 mL, 33.4 mmol) were added and the mixture was stirred 30 minutes, washed with a saturated aqueous ammonium chloride solution and concentrated. Flash column separation with 50% ethyl acetate/hexane gave 2-tert-butyl-N-(2-pyridin-2-ylethyl)benzenesulfonamide (3.29 g, 36%).

MS (ESI+) m/z 319;

MS (ESI−) m/z 317.

Step 2: Following the same procedure described in Example 474 (Step 2), 2-tert-butyl-N-(2-pyridin-2-ylethyl)benzenesulfonamide was used to prepare 5-bromo-2-tert-butyl-N-(2-pyridin-2-ylethyl)benzenesulfonamide.

Step 3: Following the same procedure described in Example 474 (Step 4), 5-bromo-2-tert-butyl-N-(2-pyridin-2-ylethyl)benzenesulfonamide and phenyl sulfonyl fluoride were used to prepare 2-tert-butyl-5-(phenylsulfonyl)-N-(2-pyridin-2-ylethyl)benzenesulfonamide.

MS (ESI+) m/z 459.

### EXAMPLE 462

### N-[1-(2-methoxybenzoyl)piperidin-4-yl]-2-methyl-5-(phenylsulfonyl)benzenesulfonamide

To a slurry of 2-methyl-5-(phenylsulfonyl)-N-piperidin-4-ylbenzenesulfonamide (50 mg, 0.13 mmol) and morphlinomethyl-polystyrene (170 mg, 62 mmol) in dichloromethane (8 mL) was added 2-anisolyl chloride (65 mg, 0.38 mmol). The reaction was rotated on an orbital shaker overnight. Then aminomethylated-polystyrene (150 mg, 0.33 mmol) was added to the reaction and it was rotated for addition four hours. The reaction was then filtered and the resin was washed alternately with 10% dichloromethane/methanol (3×6 mL). The crude residue was purified using automated flash chromatography with a gradient mobile phase consisting of ethyl acetate and hexane resulting in the isolation of N-[1-(2-methoxybenzoyl)piperidin-4-yl]-2-methyl-5-(phenylsulfonyl)benzenesulfonamide (52 mg, 78%).

MS (ES+) m/z 529.

### EXAMPLE 463

### N-[1-(3-methoxybenzoyl)piperidin-4-yl]-2-methyl-5-(phenylsulfonyl)benzenesulfonamide

In an analogous manner to Example 462, 2-methyl-5-(phenylsulfonyl)-N-piperidin-4-ylbenzenesulfonamide and 3-anisolyl chloride were used to prepare N-[1-(3-methoxybenzoyl)piperidin-4-yl-2-methyl-5-(phenylsulfonyl)benzenesulfonamide.

MS (ES+) m/z 529.

### EXAMPLE 464

### N-[1-(3,4-dimethoxybenzoyl)piperidin-4-yl]-2-methyl-5-(phenylsulfonyl)benzenesulfonamide

In an analogous manner to Example 462, 2-methyl-5-(phenylsulfonyl)-N-piperidin-4-ylbenzenesulfonamide and 3,4-dimethoxybenzene-1-carbonyl chloride were used to prepare N-[1-(3,4-dimethoxybenzoyl)piperidin-4-yl]-2-methyl-5-(phenylsulfonyl)benzenesulfonamide.

MS (ES+) m/z 559.

### EXAMPLE 465

### 2-methyl-5-(phenylsulfonyl)-N-{1-[3-(trifluoromethyl)benzoyl]piperidin-4-yl}benzenesulfonamide

In an analogous manner to Example 462, 2-methyl-5-(phenylsulfonyl)-N-piperidin-4-ylbenzenesulfonamide and 3-(trifluoromethyl)benzoyl chloride were used to prepare 2-methyl-5-(phenylsulfonyl)-N-{1-[3-(trifluoromethyl)benzoyl]piperidin-4-yl}benzenesulfonamide.

MS (ES+) m/z 567.

### EXAMPLE 466

### N-[1-(4-chlorobenzoyl)piperidin-4-yl]-2-methyl-5-(phenylsulfonyl)benzenesulfonamide

In an analogous manner to Example 462, 2-methyl-5-(phenylsulfonyl)-N-piperidin-4-ylbenzenesulfonamide and 4-chlorobenzoic acid chloride were used to prepare N-[1-(4-chlorobenzoyl)piperidin-4-yl]-2-methyl-5-(phenylsulfonyl)benzenesulfonamide.

MS (ES+) m/z 533.

### EXAMPLE 467

### N-[1-(4-methoxybenzoyl)piperidin-4-yl]-2-methyl-5-(phenylsulfonyl)benzenesulfonamide

In an analogous manner to Example 462, 2-methyl-5-(phenylsulfonyl)-N-piperidin-4-ylbenzenesulfonamide and 4-anisoyl chloride were used to prepare N-[1-(4-methoxybenzoyl)piperidin-4-yl]-2-methyl-5-(phenylsulfonyl)benzenesulfonamide.

MS (ES+) m/z 529.

### EXAMPLE 468

### 2-methyl-N-[1-(4-methylbenzoyl)piperidin-4-yl]-5-(phenylsulfonyl)benzenesulfonamide

In an analogous manner to Example 462, 2-methyl-5-(phenylsulfonyl)-N-piperidin-4-ylbenzenesulfonamide and 4-methylbenzoyl chloride were used to prepare 2-methyl-N-[1-(4-methylbenzoyl)piperidin-4-yl]-5-(phenylsulfonyl)benzenesulfonamide.

MS (ES+) m/z 513.

### EXAMPLE 469

### N-[1-(methoxyacetyl)piperidin-4-yl]-2-methyl-5-(phenylsulfonyl)benzenesulfonamide

In an analogous manner to Example 462, 2-methyl-5-(phenylsulfonyl)-N-piperidin-4-ylbenzenesulfonamide and methoxyacetic acid chloride were used to prepare N-[1-(methoxyacetyl)piperidin-4-yl]-2-methyl-5-(phenylsulfonyl)benzenesulfonamide.

MS (ES+) m/z 467.

### EXAMPLE 470

### 2-methyl-N-[1-(phenylacetyl)piperidin-4-yl]-5-(phenylsulfonyl)benzenesulfonamide

In an analogous manner to Example 462, 2-methyl-5-(phenylsulfonyl)-N-piperidin-4-ylbenzenesulfonamide and α-toluyl chloride were used to prepare 2-methyl-N-[1-(phenylacetyl)piperidin-4-yl]-5-(phenylsulfonyl)benzenesulfonamide.

MS (ES+) m/z 513.

### EXAMPLE 471

### N-[1-(cyclohexylcarbonyl)piperidin-4-yl]-2-methyl-5-(phenylsulfonyl)benzenesulfonamide

In an analogous manner to Example 462, 2-methyl-5-(phenylsulfonyl)-N-piperidin-4-ylbenzenesulfonamide and cyclohexanecarbonyl chloride were used to prepare N-[1-(cyclohexylcarbonyl)piperidin-4-yl]-2-methyl-5-(phenylsulfonyl)benzenesulfonamide.

MS (ES+) m/z 505.

### EXAMPLE 472

### 2,6-dimethyl-3-(phenylsulfonyl)-N-(2-pyridin-2-ylethyl)benzenesulfonamide

Step 1: Following the same procedure described in Example 1 (Step 1), 2-bromo-1,3-dimethyl benzene was used to prepare 3-bromo-2,4-dimethylbenzenesulfonyl chloride.

Step 2: Following the same procedure described in Example 230 (Step 1), 3-bromo-2,4-dimethylbenzenesulfonyl chloride was used to prepare 2-bromo-1,3-dimethyl-4-(phenylsulfonyl)benzene.

Step 3: Following the same procedure described in Example 474 (Step 1), 2-bromo-1,3-dimethyl-4-(phenylsulfonyl)benzene was used to prepare 2,6-dimethyl-3-(phenylsulfonyl)-N-(2-pyridin-2-ylethyl)benzenesulfonamide.

MS (ESI+) m/z 431;

MS (ESI−) m/z 429.

### EXAMPLE 473

### 2,6-dimethyl-3-(phenylsulfonyl)-N-(tetrahydro-2H-pyran-4-yl)benzenesulfonamide

In an analogous manner to Example 472,

Step 3: tetrahydropyran-4-ylamine was used to prepare 2,6-dimethyl-3-(phenylsulfonyl)-N-(tetrahydro-2H-pyran-4-yl)benzenesulfonamide.

MS (ESI+) m/z 410;

MS (ESI−) m/z 408.

### EXAMPLE 474

### 5-{[5-(dimethylamino)-1-naphthyl]sulfonyl}-2-isopropyl-N-(2-pyridin-2-ylethyl)benzenesulfonamide

Step 1: To a stirred solution of 2-bromoisopropylbenzene (10.0 g, 50.2 mmol) in THF (350 mL) at −78° C. was added n-butyl lithium 2.5 M in hexane (14.0 mL, 35.0 mmol) dropwise. After 10 minutes, sulfur dioxide was bubbled in over a period of 30 minutes. The solution was allowed to warm to room temperature and stirred for 2 hours. The solution was concentrated and taken up in methylene chloride (300 mL). N-chlorosuccinimide (8.1 g, 60.6 mmol) was added and the solution was stirred 1.5 hours at room temperature. 2-(2-aminoethyl)pyridine (7.2 mL, 60.2 mmol) was added and the solution was stirred an additional 45 minutes at room temperature, washed with a saturated aqueous ammonium chloride solution. The organic layer was dried over magnesium sulfate and concentrated. Trituration with ether gave 2-isopropyl-N-(2-pyridin-2-ylethyl)benzenesulfonamide (8.67 g, 56%).

MS (ESI) m/z 305.

Step 2: To a stirred solution of 2-isopropyl-N-(2-pyridin-2-ylethyl)benzenesulfonamide (10.0 g, 32.8 mmol) in 90% concentrated sulfuric acid (110 mL) was added N-bromosuccinimide (5.85 g, 32.8 mmol) at room temperature and the solution was stirred 15 minutes. The solution was chilled in an ice bath and 25% sodium hydroxide was slowly added to pH 9. The solution was extracted several times with ethyl acetate. The organic layers were combined, dried over magnesium sulfate, and concentrated. Trituration with ether gave 5-bromo-2-isopropyl-N-(2-pyridin-2-ylethyl)benzenesulfonamide (6.0 g, 48%).

MS (ESI+) m/z 383;

MS (ESI−) m/z 381.

Step 3: To a stirred solution of dansyl chloride (1.0 g, 3.70 mmol) in acetonitrile (3 mL), was added potassium fluoride (0.86 g, 14.8 mmol) and 18-crown-6 (0.05 g, 0.19 mmol). The resulting solution was stirred overnight at room temperature. The solution was diluted with D.I. water and extracted several times with ethyl acetate. The combined organic layers were dried over magnesium sulfate and concentrated to give dansyl fluoride (0.50 g, 53%).

Step 4: To a stirred solution of 5-bromo-2-isopropyl-N-(2-pyridin-2-ylethyl)benzenesulfonamide (0.30 g, 0.78 mmol) in THF (3 mL) at 0° C. was added methyl magnesium bromide 1.4 M in 75% toluene/THF (0.6 mL, 0.84 mmol). The reaction was stirred 10 minutes, then cooled to −78° C. and n-butyl lithium 2.5 M in hexane (0.35 mL, 0.87 mmol) was added dropwise. The reaction was stirred an additional 10 minutes. The reaction was warmed to 0° C. and dansyl fluoride (0.20 g, 0.79 mmol) was added. The reaction was stirred overnight at room temperature, partitioned between ammonium chloride solution and ethyl acetate. The organic layer was concentrated and HPLC separated to give 5-{[5-(dimethylamino)-1-naphthyl]sulfonyl}-2-isopropyl-N-(2-pyridin-2-ylethyl)benzenesulfonamide (0.05 g, 12%).

MS (ESI+) m/z 538;

MS (ESI−) m/z 536.

### EXAMPLE 475

### N-[1-(cyclopropylcarbonyl)piperidin-4-yl]-2-methyl-5-(phenylsulfonyl)benzenesulfonamide

In an analogous manner to Example 462, 2-methyl-5-(phenylsulfonyl)-N-piperidin-4-ylbenzenesulfonamide and cyclopropanecarbonyl chloride were used to prepare N-[1-(cyclopropylcarbonyl)piperidin-4-yl]-2-methyl-5-(phenylsulfonyl)benzenesulfonamide.

MS (ES+) m/z 463.

### EXAMPLE 476

### N-[1-(4-cyanobenzoyl)piperidin-4-yl]-2-methyl-5-(phenylsulfonyl)benzenesulfonamide

In an analogous manner to Example 462, 2-methyl-5-(phenylsulfonyl)-N-piperidin-4-ylbenzenesulfonamide and 4-(chlorocarbonyl)benzonitrile were used to prepare N-[1-(4-cyanobenzoyl)piperidin-4-yl]-2-methyl-5-(phenylsulfonyl)benzenesulfonamide.

MS (ES+) m/z 524.

### EXAMPLE 477

### N-[1-(3-cyanobenzoyl)piperidin-4-yl]-2-methyl-5-(phenylsulfonyl)benzenesulfonamide

In an analogous manner to Example 462, 2-methyl-5-(phenylsulfonyl)-N-piperidin-4-ylbenzenesulfonamide and 3-cyanobenzoyl chloride were used to prepare N-[1-(3-cyanobenzoyl)piperidin-4-yl]-2-methyl-5-(phenylsulfonyl)benzenesulfonamide.

MS (ES+) m/z 524.

### EXAMPLE 478

### 2-methyl-N-[1-(methylsulfonyl)piperidin-4-yl]-5-(phenylsulfonyl)benzenesulfonamide

In an analogous manner to Example 462, 2-methyl-5-(phenylsulfonyl)-N-piperidin-4-ylbenzenesulfonamide and methanesulfonyl chloride were used to prepare 2-methyl-N-[1-(methylsulfonyl)piperidin-4-yl]-5-(phenylsulfonyl)benzenesulfonamide.

MS (ES+) m/z 473.

### EXAMPLE 479

### N-(1-acetylpiperidin-4-yl)-2-methyl-5-(phenylsulfonyl)benzenesulfonamide

In an analogous manner to Example 462, 2-methyl-5-(phenylsulfonyl)-N-piperidin-4-ylbenzenesulfonamide and acetyl chloride were used to prepare N-(1-acetylpiperidin-4-yl)-2-methyl-5-(phenylsulfonyl)benzenesulfonamide.

MS (ES+) m/z 437.

### EXAMPLE 480

### N-(4-{[4-({[2-methyl-5-(phenylsulfonyl)phenyl]sulfonyl}amino)piperidin-1-yl]carbonyl}phenyl)acetamide

In an analogous manner to Example 462, 2-methyl-5-(phenylsulfonyl)-N-piperidin-4-ylbenzenesulfonamide and 4-acetamidobenzoyl chloride were used to prepare N-(4-{[4-({[2-methyl-5-(phenylsulfonyl)phenyl]sulfonyl}amino)piperidin-1-yl]carbonyl}phenyl)acetamide.

MS (ES+) m/z 556.

### EXAMPLE 481

### 2-methyl-N-{1-[(1-methyl-1H-imidazol-4-yl)sulfonyl]piperidin-4-yl}-5-(phenylsulfonyl)benzenesulfonamide

In an analogous manner to Example 462, 2-methyl-5-(phenylsulfonyl)-N-piperidin-4-ylbenzenesulfonamide and 1-methylimidazole-4-sulfonyl chloride were used to prepare 2-methyl-N-{1-[(1-methyl-1H-imidazol-4-yl)sulfonyl]piperidin-4-yl}-5-(phenylsulfonyl)benzenesulfonamide.

MS (ES+) m/z 539.

### EXAMPLE 482

### 2-methyl-5-(phenylsulfonyl)-N-[1-(2-thienylsulfonyl)piperidin-4-yl]benzenesulfonamide

In an analogous manner to Example 462, 2-methyl-5-(phenylsulfonyl)-N-piperidin-4-ylbenzenesulfonamide and 2-thiophenesulfonyl chloride were used to prepare 2-methyl-5-(phenylsulfonyl)-N-[1-(2-thienylsulfonyl)piperidin-4-yl]benzenesulfonamide.

MS (ES+) m/z 541.

### EXAMPLE 483

### N-(1-{[5-(dimethylamino)-1-naphthyl]sulfonyl}piperidin-4-yl)-2-methyl-5-(phenylsulfonyl)benzenesulfonamide

In an analogous manner to Example 462, 2-methyl-5-(phenylsulfonyl)-N-piperidin-4-ylbenzenesulfonamide and dansyl chloride were used to prepare N-(1-{[5-(dimethylamino)-1-naphthyl]sulfonyl}piperidin-4-yl)-2-methyl-5-(phenylsulfonyl)benzenesulfonamide.

MS (ES+) m/z 628.

### EXAMPLE 484

### N-[1-(1,3-benzodioxol-5-ylcarbonyl)piperidin-4-yl]-2-methyl-5-(phenylsulfonyl)benzenesulfonamide

In an analogous manner to Example 462, 2-methyl-5-(phenylsulfonyl)-N-piperidin-4-ylbenzenesulfonamide and 1,3-benzodioxole-5-carbonyl chloride were used to prepare N-[1-(1,3-benzodioxol-5-ylcarbonyl)piperidin-4-yl]-2-methyl-5-(phenylsulfonyl)benzenesulfonamide.

MS (ES+) m/z 543

MS (ES−) m/z 541.

### EXAMPLE 485

### N-[1-(isoxazol-5-ylcarbonyl)piperidin-4-yl]-2-methyl-5-(phenylsulfonyl)benzenesulfonamide

In an analogous manner to Example 462, 2-methyl-5-(phenylsulfonyl)-N-piperidin-4-ylbenzenesulfonamide and 5-isoxazolecarbonyl chloride were used to prepare N-[1-(isoxazol-5-ylcarbonyl)piperidin-4-yl]-2-methyl-5-(phenylsulfonyl)benzenesulfonamide.

MS (ES+) m/z 490.

### EXAMPLE 486

### N-[1-(N,N-dimethylglycyl)piperidin-4-yl]-2-methyl-5-(phenylsulfonyl)benzenesulfonamide

In an analogous manner to Example 462, 2-methyl-5-(phenylsulfonyl)-N-piperidin-4-ylbenzenesulfonamide and dimethylaminoacetyl chloride hydrochloride were used to prepare N-[1-(N,N-dimethylglycyl)piperidin-4-yl]-2-methyl-5-(phenylsulfonyl)benzenesulfonamide.

MS (ES+) m/z 480.

### EXAMPLE 487

### prop-2-yn-1-yl 4-({[2-methyl-5-(phenylsulfonyl)phenyl]sulfonyl}amino)piperidine-1-carboxylate

In an analogous manner to Example 462, 2-methyl-5-(phenylsulfonyl)-N-piperidin-4-ylbenzenesulfonamide and propargylchloroformate were used to prepare prop-2-yn-1-yl 4-({[2-methyl-5-(phenylsulfonyl)phenyl]sulfonyl}amino)piperidine-1-carboxylate.

MS (ES+) m/z 477

MS (ES−) m/z 475.

### EXAMPLE 488

### methyl 4-({[2-methyl-5-(phenylsulfonyl)phenyl]sulfonyl}amino)piperidine-1-carboxylate

In an analogous manner to Example 462, 2-methyl-5-(phenylsulfonyl)-N-piperidin-4-ylbenzenesulfonamide and methylchloroformate were used to prepare methyl 4-({[2-methyl-5-(phenylsulfonyl)phenyl]sulfonyl}amino)piperidine-1-carboxylate.

MS (ES+) m/z 453

MS (ES−) m/z 451.

### EXAMPLE 489

### 2-methoxyphenyl 4-({[2-methyl-5-(phenylsulfonyl)phenyl]sulfonyl}amino)piperidine-1-carboxylate

In an analogous manner to Example 462, 2-methyl-5-(phenylsulfonyl)-N-piperidin-4-ylbenzenesulfonamide and 2-methoxyphenylchloroformate were used to prepare 2-methoxyphenyl 4-({[2-methyl-5-(phenylsulfonyl)phenyl]sulfonyl}amino)piperidine-1-carboxylate.

MS (ES+) m/z 545

MS (ES−) m/z 543.

### EXAMPLE 490

### N-(tert-butyl)-4-({[2-methyl-5-(phenylsulfonyl)phenyl]sulfonyl}amino)piperidine-1-carboxamide

In an analogous manner to Example 462, 2-methyl-5-(phenylsulfonyl)-N-piperidin-4-ylbenzenesulfonamide and t-butylisocyanate were used to prepare N-(tert-butyl)-4-({[2-methyl-5-(phenylsulfonyl)phenyl]sulfonyl}amino)piperidine-1-carboxamide.

MS (ES+) m/z 494

MS (ES−) m/z 492.

### EXAMPLE 491

### N-cyclohexyl-4-({[2-methyl-5-(phenylsulfonyl)phenyl]sulfonyl}amino)piperidine-1-carboxamide

In an analogous manner to Example 462, 2-methyl-5-(phenylsulfonyl)-N-piperidin-4-ylbenzenesulfonamide and cyclohexylisocyanate were used to prepare N-cyclohexyl-4-({[2-methyl-5-(phenylsulfonyl)phenyl]sulfonyl}amino)piperidine-1-carboxamide.

MS (ES+) m/z 520;

MS (ES−) m/z 518.

### EXAMPLE 492

### 5-[(3-chloro-5-cyanophenyl)sulfonyl]-2-methyl-N-(2-pyridin-2-ylethyl)benzenesulfonamide

In an analogous manner to Example 409,

Step 3: 3-chloro-5-cyanophenyl boronic acid was used to prepare 5-[(3-chloro-5-cyanophenyl)sulfonyl]-2-methyl-N-(2-pyridin-2-ylethyl)benzenesulfonamide.

MS (ES−) m/z 474.0.

### EXAMPLE 493

### N-[3-(1H-imidazol-1-yl)propyl]-2-methyl-3-(phenylsulfonyl)benzenesulfonamide

In an analogous manner to Example 302, 1-(3-aminopropyl)-imidazole was used to prepare N-[3-(1H-imidazol-1-yl)propyl]-2-methyl-3-(phenylsulfonyl)benzenesulfonamide.

MS (ES−) m/z 417.9;

HRMS: calcd for C₁₉H₂₁N₃O₄S₂+H+, 420.10462; found (ESI, [M+H]⁺), 420.1049.

### EXAMPLE 494

### 2-methyl-3-(phenylsulfonyl)-N-(2-pyridin-2-ylethyl)benzenesulfonamide

In an analogous manner to Example 302, 2-(2-aminoethyl)-pyridine was used to prepare 2-methyl-3-(phenylsulfonyl)-N-(2-pyridin-2-ylethyl)benzenesulfonamide.

MS (ES−) m/z 414.9;

HRMS: calcd for C₂₀H₂₀N₂O₄S₂+H+, 417.09372; found (ESI, [M+H]⁺), 417.0934.

### EXAMPLE 495

### 2-methyl-N-(2-morpholin-4-ylethyl)-3-(phenylsulfonyl)benzenesulfonamide

In an analogous manner to Example 302, 1-ethylaminomorpholine was used to prepare 2-methyl-N-(2-morpholin-4-ylethyl)-3-(phenylsulfonyl)benzenesulfonamide (0.28 g, 2%).

MS (ES−) m/z 422.9;

HRMS: calcd for C₁₉H₂₄N₂O₅S₂+H+, 425.11994; found (ESI, [M+H]⁺), 425.1211.

### EXAMPLE 496

### 2-methyl-N-[1-(2-naphthoyl)piperidin-4-yl]-5-(phenylsulfonyl)benzenesulfonamide

In an analogous manner to Example 462, 2-methyl-5-(phenylsulfonyl)-N-piperidin-4-ylbenzenesulfonamide and 2-naphthalenecarbonyl chloride were used to prepare 2-methyl-N-[1-(2-naphthoyl)piperidin-4-yl]-5-(phenylsulfonyl)benzenesulfonamide.

MS (ES+) m/z 549

MS (ES−) m/z 547

HRMS: calcd. for C₂₉H₂₈N₂O₅S₂+H⁺, 549.15124: found (ESI, [m+H]⁺), 549.15026.

### EXAMPLE 497

### 2-methyl-5-(phenylsulfonyl)-N-[1-(2-thienylcarbonyl)piperidin-4-yl]benzenesulfonamide

In an analogous manner to Example 462, 2-methyl-5-(phenylsulfonyl)-N-piperidin-4-ylbenzenesulfonamide and 2-thenoyl chloride were used to prepare 2-methyl-5-(phenylsulfonyl)-N-[1-(2-thienylcarbonyl)piperidin-4-yl]benzenesulfonamide.

MS (ES+) m/z 505

MS (ES−) m/z 503

HRMS: calcd. for C₂₃H₂₄N₂O₅S₃+H⁺; 505.092564: found (ESI, [m+H]⁺), 505.0947.

### EXAMPLE 498

### isobutyl 4-({[2-methyl-5-(phenylsulfonyl)phenyl]sulfonyl}amino)piperidine-1-carboxylate

In an analogous manner to Example 462, 2-methyl-5-(phenylsulfonyl)-N-piperidin-4-ylbenzenesulfonamide and isobutyl chloroformate were used to prepare isobutyl 4-({[2-methyl-5-(phenylsulfonyl)phenyl]sulfonyl}amino)piperidine-1-carboxylate.

MS (ES+) m/z 495

MS (ES−) m/z 493

HRMS: calcd. for C₂₃H₃₀N₂O₆S₂+H⁺, 495.16181: found (ESI, [m+H]⁺), 495.16266.

### EXAMPLE 499

### N-{1-[4-(dimethylamino)benzoyl]piperidin-4-yl}-2-methyl-5-(phenylsulfonyl)benzenesulfonamide

In an analogous manner to Example 462, 2-methyl-5-(phenylsulfonyl)-N-piperidin-4-ylbenzenesulfonamide and 4-dimethylaminobenzoyl chloride were used to prepare N-{1-[4-(dimethylamino)benzoyl]piperidin-4-yl -2-methyl-5-(phenylsulfonyl)benzenesulfonamide.

MS (ES+) m/z 542

MS (ES−) m/z 540

HRMS: calcd. for C₂₇H₃₁N₃O₅S₂+H⁺, 542.17779: found (ESI, [m+H]⁺), 542.17725.

### EXAMPLE 500

### 4-fluorophenyl 4-({[2-methyl-5-(phenylsulfonyl)phenyl]sulfonyl}amino)piperidine-1-carboxylate

In an analogous manner to Example 462, 2-methyl-5-(phenylsulfonyl)-N-piperidin-4-ylbenzenesulfonamide and 4-fluorophenylchloroformate were used to prepare 4-fluorophenyl 4-({[2-methyl-5-(phenylsulfonyl)phenyl]sulfonyl}amino)piperidine-1-carboxylate.

MS (ES+) m/z 533

MS (ES−) m/z 531

HRMS: calcd. for C₂₅H₂₅FN₂O₆S₂+H⁺, 533.12109: found (ESI, [m+H]⁺), 533.12157.

### EXAMPLE 501

### N-ethyl-4-({[2-methyl-5-(phenylsulfonyl)phenyl]sulfonyl}amino)piperidine-1-carboxamide

In an analogous manner to Example 462, 2-methyl-5-(phenylsulfonyl)-N-piperidin-4-ylbenzenesulfonamide and ethylisocyanate were used to prepare N-ethyl-4-({[2-methyl-5-(phenylsulfonyl)phenyl]sulfonyl}amino)piperidine-1-carboxamide.

MS (ES+) m/z 466

MS (ES−) m/z 464

HRMS: calcd. for C₂₁H₂₇N₃O₅S₂+H⁺, 466.14649: found (ESI, [m+H]⁺), 466.14684.

### EXAMPLE 502

### 2-methyl-N-[1-(morpholin-4-ylcarbonyl)piperidin-4-yl]-5-(phenylsulfonyl)benzenesulfonamide

In an analogous manner to Example 462, 2-methyl-5-(phenylsulfonyl)-N-piperidin-4-ylbenzenesulfonamide and 4-morpholinecarbonyl chloride were used to prepare 2-methyl-N-[1-(morpholin-4-ylcarbonyl)piperidin-4-yl]-5-(phenylsulfonyl)benzenesulfonamide.

MS (ES+) m/z 508

MS (ES−) m/z 506

HRMS: calcd. for C₂₃H₂₉N₃O₆S₂+H⁺, 508.15706: found (ESI, [m+H]⁺), 508.15678.

### EXAMPLE 503

### N,N-dimethyl-4-({[2-methyl-5-(phenylsulfonyl)phenyl]sulfonyl}amino)piperidine-1-carboxamide

In an analogous manner to Example 462, 2-methyl-5-(phenylsulfonyl)-n-piperidin-4-ylbenzenesulfonamide and dimethylcarbamyl chloride were used to prepare N,N-dimethyl-4-({[2-methyl-5-(phenylsulfonyl)phenyl]sulfonyl}amino)piperidine-1-carboxamide.

MS (ES+) m/z 466

MS (ES−) m/z 464

HRMS: calcd. for C₂₁H₂₇N₃O₅S₂+H⁺, 466.14649: found (ESI, [m+H]⁺), 466.14672.

### EXAMPLE 504

### N-[1-(3,3-dimethylbutanoyl)piperidin-4-yl]-2-methyl-5-(phenylsulfonyl)benzenesulfonamide

In an analogous manner to Example 462, 2-methyl-5-(phenylsulfonyl)-n-piperidin-4-ylbenzenesulfonamide and t-butylacetyl chloride were used to prepare N-[1-(3,3-dimethylbutanoyl)piperidin-4-yl]-2-methyl-5-(phenylsulfonyl)benzenesulfonamide.

MS (ES+) m/z 493

MS (ES−) m/z 491

HRMS: calcd. for C₂₄H₃₂N₂O₅S₂+H⁺, 493.183092: found (ESI, [m+H]⁺), 493.1813.

### EXAMPLE 505

### 5-[(3,5-dichlorophenyl)sulfonyl]-2-isopropyl-N-(2-pyridin-2-ylethyl)benzenesulfonamide

Step 1: Following the same procedure described in Example 474 (Step 3), 3,5-dichlorobenzene sulfonyl chloride was used to prepare 3,5-dichlorobenzene sulfonyl fluoride.

Step 2: Following the same procedure described in Example 474 (Step 4), 5-bromo-2-isopropyl-N-(2-pyridin-2-ylethyl)benzenesulfonamide and 3,5-dichlorobenzene sulfonyl fluoride were used to prepare 5-[(3,5-dichlorophenyl)sulfonyl]-2-isopropyl-N-(2-pyridin-2-ylethyl)benzenesulfonamide.

MS (ESI) m/z 513;

MS (ESI) m/z 511;

HRMS: calcd for C₂₂H₂₂Cl₂N₂O₄S₂+H+, 513.04708; found (ESI-FTMS, [M+H]¹⁺), 513.04709.

### EXAMPLE 506

### 2-isopropyl-N-(2-pyridin-2-ylethyl)-5-{[3-(trifluoromethoxy)phenyl]sulfonyl}benzenesulfonamide

In an analogous manner to Example 454,

Step 2: 3-trifluoromethoxyphenyl boronic acid was used to prepare 2-isopropyl-N-(2-pyridin-2-ylethyl)-5-{[3-(trifluoromethoxy)phenyl]sulfonyl}benzenesulfonamide.

MS (EST) m/z 529;

MS (ESI) m/z 527;

HRMS: calcd for C23H₂₃F₃N₂O₅S₂+H+, 529.10732; found (ESI-FTMS, [M+H]¹⁺), 529.10719.

### EXAMPLE 507

### 5-{[4-(dimethylamino)phenyl]sulfonyl}-2-isopropyl-N-(2-pyridin-2-ylethyl)benzenesulfonamide

In an analogous manner to Example 454,

Step 2: 4-dimethylaminophenyl boronic acid was used to prepare 5-{[4-(dimethylamino)phenyl]sulfonyl}-2-isopropyl-N-(2-pyridin-2-ylethyl)benzenesulfonamide.

MS (ESI) m/z 488;

MS (ESI) m/z 486;

HRMS: calcd for C₂₄H₂₉N₃O₄S₂+H+, 488.16722; found (ESI-FTMS, [M+H]¹⁺), 488.16721.

### EXAMPLE 508

### 2-isopropyl-N-(2-pyridin-2-ylethyl)-5-{[3-(trifluoromethyl)phenyl]sulfonyl}benzenesulfonamide

Step 1: Following the same procedure described in Example 474 (Step 3), 3-trifluoromethylbenzene sulfonyl chloride was used to prepare 3-trifluoromethylbenzene sulfonyl fluoride.

Step 2: Following the same procedure described in Example 474 (Step 4), 5-bromo-2-isopropyl-N-(2-pyridin-2-ylethyl)benzenesulfonamide and 3-trifluoromethylbenzene sulfonyl fluoride were used to prepare 2-isopropyl-N-(2-pyridin-2-ylethyl)-5-{[3-(trifluoromethyl)phenyl]sulfonyl}benzenesulfonamide.

MS (ESI) m/z 513;

MS (ESI) m/z 511;

HRMS: calcd for C₂₃H₂₃F₃N₂O₄S₂+H+, 513.11241; found (ESI-FTMS, [M+H]¹⁺), 513.11169.

### EXAMLE 509

### 5-[(5-chloro-2-methoxyphenyl)sulfonyl]-2-isopropyl-N-(2-pyridin-2-ylethyl)benzenesulfonamide

Step 1: Following the same procedure described in Example 474, (Step 3), 5-chloro-2-methoxybenzene sulfonyl chloride was used to prepare 5-chloro-2-methoxybenzene sulfonyl fluoride.

Step 2: Following the same procedure described in Example 474 (Step 4), 5-bromo-2-isopropyl-N-(2-pyridin-2-ylethyl)benzenesulfonamide and 5-chloro-2-methoxybenzene sulfonyl fluoride were used to prepare 5-[(5-chloro-2-methoxyphenyl)sulfonyl]-2-isopropyl-N-(2-pyridin-2-ylethyl)benzenesulfonamide.

MS (ESI) m/z 509;

MS (ESI) m/z 507;

HRMS: calcd for C₂₃H₂₅ClN₂O₅S₂+H+, 509.09662; found (ESI-FTMS, [M+H]¹⁺), 509.0966.

### EXAMPLE 510

### 2-isopropyl-N-(2-pyridin-2-ylethyl)-5-(quinolin-8-ylsulfonyl)benzenesulfonamide

Step 1: Following the same procedure described in Example 474, (Step 3), quinolin-8-sulfonyl chloride was used to prepare quinolin-8-sulfonyl fluoride.

Step 2: Following the same procedure described in Example 474, (Step 4), 5-bromo-2-isopropyl-N-(2-pyridin-2-ylethyl)benzenesulfonamide and quinolin-8-sulfonyl fluoride were used to prepare 2-isopropyl-N-(2-pyridin-2-ylethyl)-5-(quinolin-8-ylsulfonyl)benzenesulfonamide.

MS (ESI) m/z 496;

MS (ESI) m/z 494;

HRMS: calcd for C₂₅H₂₅N₃O₄S₂+H+, 496.13592; found (ESI-FTMS, [M+H]¹⁺), 496.13588.

### EXAMPLE 511

### 5-[(2,5-dichloro-3-thienyl)sulfonyl]-2-isopropyl-N-(2-pyridin-2-ylethyl)benzenesulfonamide

Step 1: Following the same procedure described in Example 474, (Step 3), 2,5-dichlorothiophene-3-sulfonyl chloride was used to prepare 2,5-dichlorothiophene-3-sulfonyl fluoride.

Step 2: Following the same procedure described in Example 474, (Step 4), 5-bromo-2-isopropyl-N-(2-pyridin-2-ylethyl)benzenesulfonamide and 2,5-dichlorothiophene-3-sulfonyl fluoride were used to prepare 5-[(2,5-dichloro-3-thienyl)sulfonyl]-2-isopropyl-N-(2-pyridin-2-ylethyl)benzenesulfonamide.

MS (ESI) m/z 519;

MS (ESI) m/z 517;

HRMS: calcd for C₂₀H₂₀Cl₂N₂O₄S₃+H+, 519.00350; found (ESI-FTMS, [M+H]¹⁺), 519.00298.

### EXAMPLE 512

### 5-[(5-chloro-1,3-dimethyl-1H-pyrazol-4-yl)sulfonyl]-2-isopropyl-N-(2-pyridin-2-ylethyl)benzenesulfonamide

Step 1: Following the same procedure described in Example 474, (Step 3), 5-chloro-1,3-dimethyl-1H-pyrazole-4-sulfonyl chloride was used to prepare 5-chloro-1,3-dimethyl-1H-pyrazole-4-sulfonyl fluoride.

Step 2: Following the same procedure described in Example 474, (Step 4), 5-bromo-2-isopropyl-N-(2-pyridin-2-ylethyl)benzenesulfonamide and 5-chloro-1,3-dimethyl-1H-pyrazole-4-sulfonyl fluoride were used to prepare 5-[(5-chloro-1,3-dimethyl-1H-pyrazol-4-yl)sulfonyl]-2-isopropyl-N-(2-pyridin-2-ylethyl)benzenesulfonamide.

MS (ESI) m/z 497;

MS (ESI) m/z 495;

HRMS: calcd for C₂₁H₂₅ClN40₄S₂+H+, 497.10785; found (ESI, [M+H]⁺), 497.1062.

### EXAMPLE 513

### 2-isopropyl-5-[(1-methyl-1H-imidazol-4-yl)sulfonyl]-N-(2-pyridin-2-ylethyl)benzenesulfonamide

Step 1: Following the same procedure described in Example 474, (Step 3), 1-methyl-1H-imidazole-4-sulfonyl chloride was used to prepare 1-methyl-1H-imidazole-4-sulfonyl fluoride.

Step 2: Following the same procedure described in Example 474, (Step 4), 5-bromo-2-isopropyl-N-(2-pyridin-2-ylethyl)benzenesulfonamide and 1-methyl-1H-imidazole-4-sulfonyl fluoride were used to prepare 2-isopropyl-5-[(1-methyl-1H-imidazol-4-yl)sulfonyl]-N-(2-pyridin-2-ylethyl)benzenesulfonamide.

MS (ESI) m/z 449;

MS (ESI) m/z 447;

HRMS: calcd for C₂₀H₂₄N₄O₄S₂+H+, 449.13117; found (ESI, [M+H]⁺), 449.133.

### EXAMPLE 514

### 5-[(3-chloro-2-methylphenyl)sulfonyl]-2-isopropyl-N-(2-pyridin-2-ylethyl)benzenesulfonamide

Step 1: Following the same procedure described in Example 474, (Step 3), 3-chloro-2-methylbenzene sulfonyl chloride was used to prepare 3-chloro-2-methylbenzene sulfonyl fluoride.

Step 2: Following the same procedure described in Example 474, (Step 4), 5-bromo-2-isopropyl-N-(2-pyridin-2-ylethyl)benzenesulfonamide and 3-chloro-2-methylbenzene sulfonyl fluoride were used to prepare 5-[(3-chloro-2-methylphenyl)sulfonyl]-2-isopropyl-N-(2-pyridin-2-ylethyl)benzenesulfonamide.

MS (ESI) m/z 493;

MS (ESI) m/z 491;

HRMS: calcd for C₂₃H₂₅ClN₂O₄S₂+H+, 493.10170; found (ESI, [M+H]⁺), 493.1028.

### EXAMPLE 515

### 5-[(4,4-dimethyl-2-oxo-1,4-dihydro-2H-3,1-benzoxazin-6-yl)sulfonyl]-2-isopropyl-N-(2-pyridin-2-ylethyl)benzenesulfonamide

In an analogous manner to Example 454,

Step 2: 4,4-dimethyl-2-oxo-1,4-dihydro-2H-3,1-benzoxazin-6-boronic acid was used to prepare 5-[(4,4-dimethyl-2-oxo-1,4-dihydro-2H-3,1-benzoxazin-6-yl)sulfonyl]-2-isopropyl-N-(2-pyridin-2-ylethyl)benzenesulfonamide.

MS (ES) m/z 542.0;

HRMS: calcd for C₂₆H₂₉N₃O₆S₂+H+, 544.15705; found (ESI-FTMS, [M+H]¹⁺), 544.1574;

### EXAMPLE 516

### 2-methyl-5-(phenylsulfonyl)-N-[1-(pyridin-3-ylcarbonyl)piperidin-4-yl]benzenesulfonamide

In an analogous manner to Example 462, 2-methyl-5-(phenylsulfonyl)-n-piperidin-4-ylbenzenesulfonamide and 3-pyridinecarbonyl chloride were used to prepare 2-methyl-5-(phenylsulfonyl)-N-[1-(pyridin-3-ylcarbonyl)piperidin-4-yl]benzenesulfonamide.

MS (ES+) m/z 500

MS (ES−) m/z 498

HRMS: calcd. for C₂₄H₂₅N₃O₅S₂+H⁺, 500.131391: found (ESI, [m+H]⁺), 500.1339.

### EXAMPLE 517

### tert-butyl 4-[({5-[(4-fluorophenyl)sulfonyl]-2-isopropylphenyl}sulfonyl)amino]piperidine-1-carboxylate

In an analogous manner to Example 435, 5-(4-fluoro-benzenesulfonyl)-2-isopropyl-benzenesulfonyl chloride and 4-amino-1-boc-piperidine were used to prepare tert-butyl 4-[({5-[(4-fluorophenyl)sulfonyl]-2-isopropylphenyl}sulfonyl)amino]piperidine-1-carboxylate.

MS (ES−) m/z 539.

### EXAMPLE 518

### N-(1-{[5-(dimethylamino)-1-naphthyl]sulfonyl}piperidin-4-yl)-5-[(4-fluorophenyl)sulfonyl]-2-isopropylbenzenesulfonamide

In an analogous manner to Example 462, 2-isopropyl-5-(phenylsulfonyl)-n-piperidin-4-ylbenzenesulfonamide and dansyl chloride were used to prepare N-(1-{[5-(dimethylamino)-1-naphthyl]sulfonyl}piperidin-4-yl)-5-[(4-fluorophenyl)sulfonyl]-2-isopropylbenzenesulfonamide.

MS (ES+) m/z 674

MS (ES−) m/z 672.

### EXAMPLE 519

### 5-[(4-fluorophenyl)sulfonyl]-2-isopropyl-N-[1-(methoxyacetyl)piperidin-4-yl]benzenesulfonamide

In an analogous manner to Example 462, 2-isopropyl-5-(phenylsulfonyl)-n-piperidin-4-ylbenzenesulfonamide and methoxyacetic chloride were used to prepare 5-[(4-fluorophenyl)sulfonyl]-2-isopropyl-N-[1-(methoxyacetyl)piperidin-4-yl]benzenesulfonamide.

MS (ES+) m/z 513

MS (ES−) m/z 511

HRMS: calcd. for C₂₃H₂₉FN₂O₆S₂+H⁺, 513.1524: found (ESI, [m+H]⁺), 513.1525.

### EXAMPLE 520

### 5-[(4-fluorophenyl)sulfonyl]-2-isopropyl-N-piperidin-4-ylbenzenesulfonamide

tert-Butyl 4-[({5-[(4-fluorophenyl)sulfonyl]-2-isopropylphenyl}sulfonyl)amino]piperidine-1-carboxylate (0.5 mg, 0.92 mmol) was added to 3 mL of 4 M hydrochloric acid in dioxane and the precipitate was filtered giving the desired 5-[(4-fluorophenyl)sulfonyl]-2-isopropyl-N-piperidin-4-ylbenzenesulfonamide.

MS (ES−) m/z 539

HRMS: calcd. for C₂₅H₃₃FN₂O₆S₂+H⁺, 558.2102: found (ESI, [m+H]⁺), 558.211.

### EXAMPLE 521

### N-cyclopropyl-5-[(4-fluorophenyl)sulfonyl]-2-isopropylbenzenesulfonamide

In an analogous manner to Example 435, 5-(4-fluoro-benzenesulfonyl)-2-isopropyl-benzenesulfonyl chloride and cyclopropylamine were used to prepare N-cyclopropyl-5-[(4-fluorophenyl)sulfonyl]-2-isopropylbenzenesulfonamide.

MS (ES+) m/z 398

MS (ES−) m/z 396

HRMS: calcd. for C₁₈H₂₀FNO₄S₂+H⁺, 398.0891: found (ESI, [m+H]⁺), 398.091.

### EXAMPLE 522

### N-cyclobutyl-5-[(4-fluorophenyl)sulfonyl]-2-isopropylbenzenesulfonamide

In an analogous manner to Example 435, 5-(4-fluoro-benzenesulfonyl)-2-isopropyl-benzenesulfonyl chloride and cyclobutylamine were used to prepare N-cyclobutyl-5-[(4-fluorophenyl)sulfonyl]-2-isopropylbenzenesulfonamide.

MS (ES+) m/z 412

MS (ES−) m/z 410

HRMS: calcd. for C₁₉H₂₂FNO₄S₂+H⁺, 412.1047: found (ESI, [m+H]⁺), 412.1059.

### EXAMPLE 523

### N-cyclopentyl-5-[(4-fluorophenyl)sulfonyl]-2-isopropylbenzenesulfonamide

In an analogous manner to Example 435, 5-(4-fluoro-benzenesulfonyl)-2-isopropyl-benzenesulfonyl chloride and cyclopentylamine were used to prepare N-cyclopentyl-5-[(4-fluorophenyl)sulfonyl]-2-isopropylbenzenesulfonamide.

MS (ES+) m/z 426

MS (ES−) m/z 424

HRMS: calcd. for C₂₀H₂₄FNO₄S₂+H⁺, 426.1204: found (ESI, [m+H]⁺), 426.1211.

### EXAMPLE 524

### N-cyclohexyl-5-[(4-fluorophenyl)sulfonyl]-2-isopropylbenzenesulfonamide

In an analogous manner to Example 435, 5-(4-fluoro-benzenesulfonyl)-2-isopropyl-benzenesulfonyl chloride and cyclohexylamine were used to prepare N-cyclohexyl-5-[(4-fluorophenyl)sulfonyl]-2-isopropylbenzenesulfonamide.

MS (ES+) m/z 440

MS (ES−) m/z 438

HRMS: calcd. for C₂₁H₂₆FNO₄S₂+H⁺, 440.136: found (ESI, [m+H]⁺), 440.1373.

### EXAMPLE 525

### N-(1-benzylpiperidin-4-yl)-5-[(4-fluorophenyl)sulfonyl]-2-isopropylbenzenesulfonamide

In an analogous manner to Example 435, 5-(4-fluoro-benzenesulfonyl)-2-isopropyl-benzenesulfonyl chloride and 4-amino-1-benzylpiperidine were used to prepare N-(1-benzylpiperidin-4-yl)-5-[(4-fluorophenyl)sulfonyl]-2-isopropylbenzenesulfonamide.

MS (ES+) m/z 531

MS (ES−) m/z 529

HRMS: calcd. for C₂₇H₃₁FN₂O₄S₂+H⁺, 531.1782: found (ESI, [m+H]⁺), 531.1785.

### EXAMPLE 526

### ethyl 4-[({5-[(4-fluorophenyl)sulfonyl]-2-isopropylphenyl}sulfonyl)amino]piperidine-1-carboxylate

In an analogous manner to Example 435, 5-(4-fluoro-benzenesulfonyl)-2-isopropyl-benzenesulfonyl chloride and ethyl 4-amino-1-piperidine carboxylate were used to prepare ethyl 4-[({5-[(4-fluorophenyl)sulfonyl]-2-isopropylphenyl}sulfonyl)amino]piperidine-1-carboxylate.

MS (ES+) m/z 513

MS (ES−) m/z 511

HRMS: calcd. for C₂₃H₂₉FN₂O₆S₂+H⁺, 513.1524: found (ESI, [m+H]⁺), 513.1536.

### EXAMPLE 527

### 5-[(4-fluorophenyl)sulfonyl]-2-isopropyl-N-(2-piperidin-1-ylethyl)benzenesulfonamide

In an analogous manner to Example 435, 5-(4-fluoro-benzenesulfonyl)-2-isopropyl-benzenesulfonyl chloride and 1-(2-aminoethyl)piperidine were used to prepare 5-[(4-fluorophenyl)sulfonyl]-2-isopropyl-N-(2-piperidin-1-ylethyl)benzenesulfonamide.

MS (ES+) m/z 469

MS (ES−) m/z 467

HRMS: calcd. for C₂₂H₂₉FN₂O₄S₂+H⁺, 469.1626: found (ESI, [m+H]⁺), 469.1632.

### EXAMPLE 528

### tert-butyl 4-{[(2-isopropyl-5-{[4-(methylamino)phenyl]sulfonyl}phenyl)sulfonyl]amino}piperidine-1-carboxylate

To a solution of methylamine in ethanol (2.0 mL, 2.0 M) was added tert-butyl 4-[({5-[(4-fluorophenyl)sulfonyl]-2-isopropylphenyl}sulfonyl)amino]piperidine-1-carboxylate (350 mg, 0.64 mmol) and the reaction was heated to 130° C. for 30 minutes in the microwave. The reaction mixture was concentrated and purified by automated flash chromatography using a gradient of 20% to 100% ethyl acetate/hexane resulting in the isolation of 353 mg of tert-butyl 4-{[(2-isopropyl-5-{[4-(methylamino)phenyl]sulfonyl}phenyl)sulfonyl]amino}piperidine-1-carboxylate.

MS (ES−) m/z 550.

### EXAMPLE 529

### N-(1-acetylpiperidin-4-yl)-5-[(4-fluorophenyl)sulfonyl]-2-isopropylbenzenesulfonamide

In an analogous manner to Example 462, 2-isopropyl-5-(phenylsulfonyl)-n-piperidin-4-ylbenzenesulfonamide and acetyl chloride were used to prepare N-(1-acetylpiperidin-4-yl)-5-[(4-fluorophenyl)sulfonyl]-2-isopropylbenzenesulfonamide.

MS (ES+) m/z 483

MS (ES−) m/z 481

HRMS: calcd. for C₂₂H₂₇FN₂O₅S₂+H⁺, 483.142370: found (ESI, [m+H]⁺), 483.143.

### EXAMPLE 530

### N-[1-(cyclopropylcarbonyl)piperidin-4-yl]-5-[(4-fluorophenyl)sulfonyl]-2-isopropylbenzenesulfonamide

In an analogous manner to Example 462, 2-isopropyl-5-(phenylsulfonyl)-n-piperidin-4-ylbenzenesulfonamide and cyclopropylcarbonyl chloride were used to prepare N-[1-(cyclopropylcarbonyl)piperidin-4-yl]-5-[(4-fluorophenyl)sulfonyl]-2-isopropylbenzenesulfonamide.

MS (ES+) m/z 509

MS (ES−) m/z 507

HRMS: calcd. for C₂₄H₂₉FN₂O₅S₂+H⁺, 509.158019: found (ESI, [m+H]⁺), 509.1589.

### EXAMPLE 531

### N-[1-(4-cyanobenzoyl)piperidin-4-yl]-5-[(4-fluorophenyl)sulfonyl]-2-isopropylbenzenesulfonamide

In an analogous manner to Example 462, 2-isopropyl-5-(phenylsulfonyl)-N-piperidin-4-ylbenzenesulfonamide and 4-cyanobenzoyl chloride were used to prepare N-[1-(4-cyanobenzoyl)piperidin-4-yl]-5-[(4-fluorophenyl)sulfonyl]-2-isopropylbenzenesulfonamide.

MS (ES+) m/z 570

MS (ES−) m/z 568.

### EXAMPLE 532

### 5-[(4-fluorophenyl)sulfonyl]-2-isopropyl-N-(tetrahydrofuran-2-ylmethyl)benzenesulfonamide

In an analogous manner to Example 435, 5-(4-fluoro-benzenesulfonyl)-2-isopropyl-benzenesulfonyl chloride and tetrahydrofurfurylamine were used to prepare 5-[(4-fluorophenyl)sulfonyl]-2-isopropyl-N-(tetrahydrofuran-2-ylmethyl)benzenesulfonamide.

MS (ES+) m/z 442

MS (ES−) m/z 440.

### EXAMPLE 533

### 5-[(4-fluorophenyl)sulfonyl]-2-isopropyl-N-(2-pyridin-4-ylethyl)benzenesulfonamide

In an analogous manner to Example 435, 5-(4-fluoro-benzenesulfonyl)-2-isopropyl-benzenesulfonyl chloride and 4-(2-aminoethyl)pyridine were used to prepare 5-[(4-fluorophenyl)sulfonyl]-2-isopropyl-N-(2-pyridin-4-ylethyl)benzenesulfonamide.

MS (ES+) m/z 463

MS (ES−) m/z 461.

### EXAMPLE 534

### 5-[(4-fluorophenyl)sulfonyl]-2-isopropyl-N-(2-pyridin-3-ylethyl)benzenesulfonamide

In an analogous manner to Example 435, 5-(4-fluoro-benzenesulfonyl)-2-isopropyl-benzenesulfonyl chloride and 3-(2-aminoethyl)pyridine were used to prepare 5-[(4-fluorophenyl)sulfonyl]-2-isopropyl-N-(2-pyridin-3-ylethyl)benzenesulfonamide.

MS (ES+) m/z 463

MS (ES−) m/z 461

HRMS: calcd. for C₂₂H₂₃FN₂O₄S₂+H⁺, 463.116155: found (ESI, [m+H]⁺), 463.1174.

### EXAMPLE 535

### 5-({4-[(2-cyanoethyl)amino]phenyl}sulfonyl)-2-isopropyl-N-(tetrahydro-2H-pyran-4-yl)benzenesulfonamide

In an analogous manner to Example 353, 5-[(4-fluorophenyl)sulfonyl]-2-isopropyl-N-tetrahydro-2H-pyran-4-ylbenzenesulfonamide and 3-aminopropionitrile in DMA were used to prepare 5-({4-[(2-cyanoethyl)amino]phenyl}sulfonyl)-2-isopropyl-N-(tetrahydro-2H-pyran-4-yl)benzenesulfonamide.

MS (ES) m/z 490.1;

HPLC purity 96.7% at 210-370 nm, 8.3 min.; 96.9% at 304 nm, 8.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₃H₂₉N₃O₅S₂+H+, 492.16214; found (ESI, [M+H]⁺), 492.1638.

### EXAMPLE 536

### 5-({4-[(2-cyanoethyl)amino]phenyl}sulfonyl)-2-isopropyl-N-(tetrahydro-2H-pyran-4-ylmethyl)benzenesulfonamide

In an analogous manner to Example 353, 5-[(4-fluorophenyl)sulfonyl]-2-isopropyl-N-tetrahydro-2H-pyran-4-ylmethyl)benzenesulfonamide and 3-aminopropionitrile in DMA were used to prepare 5-({4-[(2-cyanoethyl)amino]phenyl}sulfonyl)-2-isopropyl-N-(tetrahydro-2H-pyran-4-ylmethyl)benzenesulfonamide.

MS (ES) m/z 504.1;

HPLC purity 96.7% at 210-370 nm, 8.5 min.; 97.0% at 304 nm, 8.5 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₄H₃₁N₃O₅S₂+H+, 506.17779; found (ESI, [M+H]⁺), 506.1786.

### EXAMPLE 537

### 5-({4-[(2-cyanoethyl)amino]phenyl}sulfonyl)-2-isopropyl-N-[2-(tetrahydro-2H-pyran-4-yl)ethyl]benzenesulfonamide

In an analogous manner to Example 353, 5-[(4-fluorophenyl)sulfonyl]-2-isopropyl-N-tetrahydro-2H-pyran-4-ylethyl)benzenesulfonamide and 3-aminopropionitrile in DMA were used to prepare 5-({4-[(2-cyanoethyl)amino]phenyl}sulfonyl)-2-isopropyl-N-[2-(tetrahydro-2H-pyran-4-yl)ethyl]benzenesulfonamide.

MS (ES) m/z 518.2;

HPLC purity 97.4% at 210-370 nm, 8.7 min.; 97.6% at 304 nm, 8.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₅H₃₃N₃O₅S₂+H+, 520.19344; found (ESI, [M+H]⁺), 520.1949.

### EXAMPLE 538

### N-(3′,6′-dihydroxy-3-oxo-3H-spiro[2-benzofuran-1,9′-xanthen]-5-yl)-4-[({5-[(4-fluorophenyl)sulfonyl]-2-isopropylphenyl}sulfonyl)amino]piperidine-1-carbothioamide

In an analogous manner to Example 462, 2-isopropyl-5-(phenylsulfonyl)-n-piperidin-4-ylbenzenesulfonamide and fluorescein isothiocyanate were used to prepare N-(3′,6′-dihydroxy-3-oxo-3H-spiro[2-benzofuran-1,9′-xanthen]-5-yl)-4-[({5-[(4-fluorophenyl)sulfonyl]-2-isopropylphenyl}sulfonyl)amino]piperidine-1-carbothioamide.

MS (ES+) m/z 830.

### EXAMPLE 539

### 5-[(3-cyanophenyl)sulfonyl]-2-isopropyl-N-(2-pyridin-2-ylethyl)benzenesulfonamide

In an analogous manner to Example 454,

Step 2: 3-cyanophenylboronic acid was used to prepare 5-[(3-cyanophenyl)sulfonyl]-2-isopropyl-N-(2-pyridin-2-ylethyl)benzenesulfonamide.

MS (ES) m/z 468.1;

HRMS: calcd for C₂₃H₂₃N₃O₄S₂+H+, 470.12027; found (ESI, [M+H]⁺), 470.1223.

### EXAMPLE 540

### 5-(1H-indol-5-ylsulfonyl)-2-isopropyl-N-(2-pyridin-2-ylethyl)benzenesulfonamide

In an analogous manner to Example 454,

Step 2: indole-5-boronic acid was used to prepare 5-(1H-indol-5-ylsulfonyl)-2-isopropyl-N-(2-pyridin-2-ylethyl)benzenesulfonamide.

MS (ES) m/z 482.1;

HRMS: calcd for C₂₄H₂₅N₃O₄S₂+H+, 484.13592; found (ESI, [M+H]⁺), 484.1382.

### EXAMPLE 541

### 2-isopropyl-5-[(2-methylphenyl)sulfonyl]-N-(tetrahydro-2H-pyran-4-yl)benzenesulfonamide

In an analogous manner to Example 474,

Step 1: 4-amino tetrahydropyran was used to prepare 2-isopropyl-N-(tetrahydro-2H-pyran-4-yl)benzenesulfonamide.

Step 2: 2-isopropyl-N-(tetrahydro-2H-pyran-4-yl)benzenesulfonamide was used to prepare 5-bromo-2-isopropyl-N-(tetrahydro-2H-pyran-4-yl)benzenesulfonamide.

Step 3: 2-methylphenyl sulfonyl fluoride and 5-bromo-2-isopropyl-N-(tetrahydro-2H-pyran-4-yl)benzenesulfonamide were used to prepare 2-isopropyl-5-[(2-methylphenyl)sulfonyl]-N-(tetrahydro-2H-pyran-4-yl)benzenesulfonamide.

MS (ES) m/z 436.1;

HRMS: calcd for C₂₁H₂₇NO₅S₂+H+, 438.14034; found (ESI, [M+H]⁺), 438.1409.

### EXAMPLE 542

### 5-[(3-chloro-2-methylphenyl)sulfonyl]-2-isopropyl-N-(tetrahydro-2H-pyran-4-yl)benzenesulfonamide

In an analogous manner to Example 541,

Step 3: 5-bromo-2-isopropyl-N-(tetrahydro-2H-pyran-4-yl)benzenesulfonamide and 3-chloro-2-methylphenyl sulfonyl fluoride were used to prepare 5-[(3-chloro-2-methylphenyl)sulfonyl]-2-isopropyl-N-(tetrahydro-2H-pyran-4-yl)benzenesulfonamide.

MS (ES) m/z 470.1;

HRMS: calcd for C₂₁H₂₆ClNO₅S₂+H+, 472.10137; found (ESI-FTMS, [M+H]¹⁺), 472.1024.

### EXAMPLE 543

### 2-isopropyl-5-{[4-(methylamino)phenyl]sulfonyl}-N-(tetrahydro-2H-pyran-4-ylmethyl)benzenesulfonamide

In an analogous manner to Example 356, 5-[(4-fluorophenyl)sulfonyl]-2-isopropyl-N-(2-tetrahydro-2H-pyran-4-ylmethyl)benzenesulfonamide and methylamine were used to prepare 2-isopropyl-5-{[4-(methylamino)phenyl]sulfonyl}-N-(tetrahydro-2H-pyran-4-ylmethyl)benzenesulfonamide.

MS (ES) m/z 465.2;

HPLC purity 96.6% at 210-370 nm, 9.0 min.; 98.2% at 310 nm, 9.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₂H₃₀N₂O₅S₂+H+, 467.16689; found (ESI, [M+H]⁺), 467.1691.

### EXAMPLE 544

### 5-{[4-(dimethylamino)phenyl]sulfonyl}-2-isopropyl-N-(tetrahydro-2H-pyran-4-yl)benzenesulfonamide

In an analogous manner to Example 356, 5-[(4-fluorophenyl)sulfonyl]-2-isopropyl-N-tetrahydro-2H-pyran-4-ylbenzenesulfonamide and a dimethylamine solution in THF were used to prepare 5-{[4-(dimethylamino)phenyl]sulfonyl}-2-isopropyl-N-(tetrahydro-2H-pyran-4-yl)benzenesulfonamide.

MS (ES) m/z 465.1;

HPLC purity 96.2% at 210-370 nm, 9.2 min.; 96.2% at 318 nm, 9.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₂H₃₀N₂O₅S₂+H+, 467.16689; found (ESI, [M+H]⁺), 467.1694.

### EXAMPLE 545

### 5-{[4-(dimethylamino)phenyl]sulfonyl}-2-isopropyl-N-(tetrahydro-2H-pyran-4-ylmethyl)benzenesulfonamide

In an analogous manner to Example 356, 5-[(4-fluorophenyl)sulfonyl]-2-isopropyl-N-(2-tetrahydro-2H-pyran-4-ylmethyl)benzenesulfonamide and a dimethylamine solution in THF were used to prepare 5-{[4-(dimethylamino)phenyl]sulfonyl}-2-isopropyl-N-(tetrahydro-2H-pyran-4-ylmethyl)benzenesulfonamide.

MS (ES) m/z 479.2;

HPLC purity 96.3% at 210-370 nm, 9.4 min.; 96.6% at 318 nm, 9.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₃H₃₂N₂O₅S₂+H+, 481.18254; found (ESI, [M+H]⁺), 481.1829.

### EXAMPLE 546

### 5-{[4-(dimethylamino)phenyl]sulfonyl}-2-isopropyl-N-[2-(tetrahydro-2H-pyran-4-yl)ethyl]benzenesulfonamide

In an analogous manner to Example 356, 5-[(4-fluorophenyl)sulfonyl]-2-isopropyl-N-(2-tetrahydro-2H-pyran-4-ylethyl)benzenesulfonamide and a dimethylamine solution in THF were used to prepare 5-{[4-(dimethylamino)phenyl]sulfonyl}-2-isopropyl-N-[2-(tetrahydro-2H-pyran-4-yl)ethyl]benzenesulfonamide.

MS (ES) m/z 493.2;

HPLC purity 100% at 210-370 nm, 9.6 min.; 99.2% at 318 nm, 9.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₄H₃₄N₂O₅S₂+H+, 495.19819; found (ESI, [M+H]⁺), 495.1986.

### EXAMPLE 547

### 2-isopropyl-5-{[4-(4-methylpiperazin-1-yl)phenyl]sulfonyl}-N-(tetrahydro-2H-pyran-4-yl)benzenesulfonamide

To a solution of 5-[(4-fluorophenyl)sulfonyl]-2-isopropyl-N-tetrahydro-2H-pyran-4-ylbenzenesulfonamide (88 mg, 0.2 mmol) in acetonitrile (0.5 mL) was added N-methylpiperazine (101 mg, 0.99 mmol). The solution was heated with stirring using microwave irradiation (150° C.) for 60 minutes. Upon cooling, the reaction mixture was diluted with water (10 mL) and the aqueous phase was extracted with ethyl acetate (3×10 mL). The combined organic phases were concentrated and the resulting crude residue was purified using automated flash column chromatography with a graduated mobile phase consisting of dichloromethane and methanol resulting in the isolation of 2-isopropyl-5-{[4-(4-methylpiperazin-1-yl)phenyl]sulfonyl}-N-(tetrahydro-2H-pyran-4-yl)benzenesulfonamide (100 mg, 97%).

MS (ES) m/z 520.2;

HPLC purity 93.9% at 210-370 nm, 6.7 min.; 95.8% at 300 nm, 6.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₅H₃₅N₃O₅S₂+H+, 522.20909; found (ESI, [M+H]⁺), 522.2094.

### EXAMPLE 548

### 2-isopropyl-5-{[4-(4-methylpiperazin-1-yl)phenyl]sulfonyl}-N-(tetrahydro-2H-pyran-4-ylmethyl)benzenesulfonamide

In an analogous manner to Example 547, 5-[(4-fluorophenyl)sulfonyl]-2-isopropyl-N-tetrahydro-2H-pyran-4-ylmethyl)benzenesulfonamide and N-methylpiperazine were used to prepare 2-isopropyl-5-{[4-(4-methylpiperazin-1-yl)phenyl]sulfonyl}-N-(tetrahydro-2H-pyran-4-ylmethyl)benzenesulfonamide.

MS (ES) m/z 534.2;

HPLC purity 94.8% at 210-370 nm, 7.0 min.; 95.2% at 300 nm, 7.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₆H₃₇N₃O₅S₂+H+, 536.22474; found (ESI, [M+H]⁺), 536.2244.

### EXAMPLE 549

### 2-isopropyl-5-{[4-(4-methylpiperazin-1-yl)phenyl]sulfonyl}-N-[2-(tetrahydro-2H-pyran-4-yl)ethyl]benzenesulfonamide

In an analogous manner to Example 547, 5-[(4-fluorophenyl)sulfonyl]-2-isopropyl-N-tetrahydro-2H-pyran-4-ylethyl)benzenesulfonamide and N-methylpiperazine were used to prepare 2-isopropyl-5-{[4-(4-methylpiperazin-1-yl)phenyl]sulfonyl}-N-[2-(tetrahydro-2H-pyran-4-yl)ethyl]benzenesulfonamide.

MS (ES) m/z 548.2;

HPLC purity 96.1% at 210-370 nm, 7.3 min.; 96.2% at 300 nm, 7.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₇H₃₉N₃O₅S₂+H+, 550.24039; found (ESI, [M+H]⁺), 550.2411.

### EXAMPLE 550

### N-(2-phenylethyl)-5-(phenylsulfonyl)-2-propylbenzenesulfonamide

### Step a) 1-(Phenylsulfonyl)-4-propylbenzene

A stirred solution of n-propylbenzene (2.40 g, 20 mmol) and benzenesulfonyl chloride (3.52 g, 20 mmol) was cooled to −40° C. and treated slowly under nitrogen with solid anhydrous aluminum chloride (3.20 g, 24 mmol). After stirring neat for 4 hours at room temperature, the mixture was slowly poured into ice water and extracted with ethyl acetate (2×). The organic phase was washed with a saturated, aqueous sodium chloride solution, dried over anhydrous sodium sulfate, and filtered through a short column of silica gel. The filtrate was evaporated in vacuo to yield a crude oil, which was crystallized from diethyl ether-hexane to yield 1-(phenylsulfonyl)4-propylbenzene (4.57 g, 88%) as a homogeneous, colorless, crystalline solid, m.p. 83-85° C.;

MS (+ESI), m/z: 261 [M+H]⁺;

MS (−ESI), m/z: 259 [M−H]⁻;

HRMS: calcd for C₁₅H₁₆O₂S, 260.08710; found (EI, M+.), 260.0876;

HPLC purity 100% at 210-370 nm, 9.8 min; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### Step b) 5-Benzenesulfonyl-2-propyl-benzenesulfonyl chloride

1-(Phenylsulfonyl)4-propylbenzene (2.60 g, 10 mmol) was heated with stirring at 60° C. for one hour under nitrogen with chlorosulfonic acid (6.7 mL, 11.7 g, 100 mmol). The mixture was cooled to room temperature, poured slowly into a cold solution of 1N hydrochloric acid, and extracted with ethyl acetate (2×.). The organic phase was washed with a saturated, aqueous sodium chloride solution, dried over anhydrous sodium sulfate, filtered, and the solvent concentrated in vacuo to a crude oil. The crude oil was crystallized from diethyl ether-hexane to afford 5-benzenesulfonyl-2-propyl-benzenesulfonyl chloride (3.51 g, 98%) as a homogeneous, colorless, crystalline solid, which was utilized in subsequent reactions.

### Step c) N-(2-Phenylethyl)-5-(phenylsulfonyl)-2-propylbenzenesulfonamide

A stirred solution of 5-benzenesulfonyl-2-propyl-benzenesulfonyl chloride (0.18 g, 0.5 mmol) in dichloromethane (10 mL) was treated dropwise under nitrogen with a solution of phenethylamine (0.12 g, 1.0 mmol) in dichloromethane. After stirring for one hour at room temperature, or until the reaction was complete, the mixture was purified by preparative liquid chromatography on a Biotage® 40 Si column of pre-packed silica gel (45 g), eluting with a gradient of 0%-25% methyl tert-butyl ether in hexane at a flow rate of 50 mL/min. After evaporation of the solvent under vacuum, the title compound, N-(2-phenylethyl)-5-(phenylsulfonyl)-2-propylbenzenesulfonamide (0.17 g, 77%), was obtained as a homogeneous, clear oil;

MS (−ESI), m/z: 442.1 [M−H]⁻;

HRMS: calcd for C₂₃H₂₅NO₄S₂+H+, 444.12978; found (ESI, [M+H]⁺), 444.1323;

HPLC purity 100% at 210-370 nm, 10.3 min; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 551

### 5-(Phenylsulfonyl)-2-propyl-N-(tetrahydro-2H-pyran-4-yl)benzenesulfonamide

The title compound was prepared from 5-benzenesulfonyl-2-propyl-benzenesulfonyl chloride (0.18 g, 0.5 mmol) and tetrahydro-pyran-4-ylamine (0.10 g, 1.0 mmol) according to the procedure and in the same manner as described in Example 550, step c. The crude product was purified by preparative liquid chromatography on a Biotage® 40 Si column of pre-packed silica gel (45 g), eluting with a gradient of 30%-70% methyl tert-butyl ether in hexane at a flow rate of 50 mL/min, to afford 5-(phenylsulfonyl)-2-propyl-N-(tetrahydro-2H-pyran-4-yl)benzenesulfonamide (0.15 g, 71%), as a homogeneous, colorless, crystalline solid, m.p. 131-133° C.;

MS (−ESI), m/z: 422.1 [M−H]⁻;

HRMS: calcd for C₂₀H₂₅NO₅S₂+H+, 424.12469; found (ESI, [M+H]⁺), 424.1262;

HPLC purity 100% at 210-370 nm, 9.0 min; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 552

### 5-(Phenylsulfonyl)-2-propyl-N-(tetrahydro-2H-pyran-4-ylmethyl)benzenesulfonamide

The title compound was prepared from 5-benzenesulfonyl-2-propyl-benzenesulfonyl chloride (0.18 g, 0.5 mmol) and (tetrahydro-pyran-4-yl)methylamine (0.12 g, 1.0 mmol) according to the procedure and in the same manner as described in Example 550, step c. The crude product was purified by preparative liquid chromatography on a Biotage® 40 Si column of pre-packed silica gel (45 g), eluting with a gradient of 30%-70% methyl tert-butyl ether in hexane at a flow rate of 50 mL/min, to afford 5-(phenylsulfonyl)-2-popyl-N-(tetrahydro-2H-pyran-4-ylmethyl)benzenesulfonamide (0.21 g, 98%), as a homogeneous, clear oil;

MS (−ESI), m/z: 436.1 [M−H]⁻;

HRMS: calcd for C₂₁H₂₇NO₅S₂+H+, 438.14034; found (ESI, [M+H]⁺), 438.1419;

HPLC purity 100% at 210-370 nm, 9.2 min; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 553

### 5-(Phenylsulfonyl)-2-propyl-N-[2-(tetrahydro-2H-pyran-4-yl)ethyl]benzenesulfonamide

The title compound was prepared from 5-benzenesulfonyl-2-propyl-benzenesulfonyl chloride (0.18 g, 0.5 mmol) and 2-(tetrahydro-pyran-4-yl)ethylamine (0.13 g, 1.0 mmol) according to the procedure and in the same manner as described in Example 550, step c. The crude product was purified by preparative liquid chromatography on a Biotage® 40 Si column of pre-packed silica gel (45 g), eluting with a gradient of 30%-70% methyl tert-butyl ether in hexane at a flow rate of 50 mL/min, to afford 5-(vhenylsulfonyl)-2-propyl-N-[2-(tetrahydro-2H-pyran-4-yl)ethyl]benzenesulfonamide (0.16 g, 72%), as a glass;

MS (−ESI), m/z: 450.1 [M−H]⁻;

HRMS: calcd for C₂₂H₂₉NO₅S₂+H+, 452.15599; found (ESI, [M+H]⁺), 452.1551;

HPLC purity 100% at 210-370 nm, 9.4 min; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 554

### N-Cyclopentyl-5-(phenylsulfonyl)-2-propylbenzenesulfonamide

The title compound was prepared from 5-benzenesulfonyl-2-propyl-benzenesulfonyl chloride (0.18 g, 0.5 mmol) and cyclopentylamine (0.09 g, 1.0 mmol) according to the procedure and in the same manner as described in Example 550, step c. The crude product was purified by preparative liquid chromatography on a Biotage® 40 Si column of pre-packed silica gel (45 g), eluting with a gradient of 0%-25% methyl tert-butyl ether in hexane at a flow rate of 50 mL/min, to afford N-cycloipentyl-5-(phenylsulfonyl)-2-propylbenzenesulfonamide (0.14 g, 67%), as a glass;

MS (−ESI), m/z: 406.1 [M−H]⁻;

HRMS: calcd for C₂₀H₂₅NO₄S₂+H+, 408.12978; found (ESI, [M+H]⁺), 408.1322;

HPLC purity 100% at 210-370 nm, 10.1 min; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 555

### 5-(Phenylsulfonyl)-2-propyl-N-(2-pyridin-2-ylethyl)benzenesulfonamide

The title compound was prepared from 5-benzenesulfonyl-2-propyl-benzenesulfonyl chloride (0.18 g, 0.5 mmol) and 2-pyridin-2-yl-ethylamine (0.12 g, 1.0 mmol) according to the procedure and in the same manner as described in Example 550, step c. The crude product was purified by preparative liquid chromatography on a Biotage® 40 Si column of pre-packed silica gel (45 g), eluting with a gradient of 50%-100% methyl tert-butyl ether in hexane at a flow rate of 50 mrlmin, to afford 5-(phenylsulfonyl)-2-propyl-N-(2-pyridin-2-ylethyl)benzenesulfonamide (0.17 g, 75%), as a homogeneous, colorless, crystalline solid, m.p. 100-102° C.;

MS (+ESI), m/z: 445 [M+H]⁺;

MS (−ESI), m/z: 443 [M−H]⁻;

HRMS: calcd for C₂₂H₂₄N₂O₄S₂+H+, 445.12502; found (ESI, [M+H]⁺), 445.1255;

HPLC purity 100% at 210-370 nm, 9.0 min; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 556

### N-[3-(1H-Imidazol-1-yl)propyl]-5-(phenylsulfonyl)-2-propylbenzenesulfonamide

The title compound was prepared from 5-benzenesulfonyl-2-propyl-benzenesulfonyl chloride (0.18 g, 0.5 mmol) and 3-imidazol-1-yl-propylamine (0.13 g, 1.0 mmol) according to the procedure and in the same manner as described in Example 550, step c. The crude product was purified by preparative liquid chromatography on a Biotage® 40 Si column of pre-packed silica gel (45 g), eluting with a gradient of 0%-4% methanol in dichloromethane at a flow rate of 50 mL/min, to afford N-[3-(1H-imidazol-1-yl)propyl]-5-(phenylsulfonyl)-2-prolpylbenzenesulfonamide (0.03 g, 14%), as a homogeneous, off-white, amorphous solid, m.p. 143-146° C.;

MS (−ESI), m/z: 443 [M−H]⁻;

HRMS: calcd for C₂₁H₂₅N₃O₄S₂+H+, 448.13592; found (ESI, [M+H]⁺), 448.1381;

HPLC purity 100% at 210-370 nm, 7.3 min; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 557

### 5-(Phenylsulfonyl)-2-propylbenzenesulfonamide

The title compound was prepared from 5-benzenesulfonyl-2-propyl-benzenesulfonyl chloride (0.18 g, 0.5 mmol) and 3-imidazol-1-yl-propylamine (0.13 g, 1.0 mmol) according to the procedure and in the same manner as described in Example 550, step c. The crude product was purified by preparative liquid chromatography on a Biotage® 40 Si column of pre-packed silica gel (45 g), eluting with a gradient of 0%-4% methanol in dichloromethane at a flow rate of 50 mL/min, to afford 5-(phenylsulfonyl)-2-propylbenzenesulfonamide (0.06 g, 34%), as a clear, tan oil;

MS (−ESI), m/z: 338.1 [M−H]⁻;

HRMS: calcd for C₁₅H₁₇NO₄S₂+NH₄⁺, 357.09372; found (ESI, [M+NH4]⁺), 357.0938;

HPLC purity 89.5% at 210-370 nm; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 558

### tert-Butyl 4-({[5-(phenylsulfonyl)-2-propylphenyl]sulfonyl}amino)piperidine-1-carboxylate

The title compound was prepared from 5-benzenesulfonyl-2-propyl-benzenesulfonyl chloride (0.36 g, 1.0 mmol) and 4-amino-piperidine-1-carboxylic acid, tert-butyl ester (0.40 g, 2.0 mmol) according to the procedure and in the same manner as described in Example 550, step c. The crude product was purified by preparative liquid chromatography on a Biotage® 40 Si column of pre-packed silica gel (45 g), eluting with a gradient of 15%-50% methyl tert-butyl ether in hexane at a flow rate of 50 mL/min, to afford tert-butyl 4-({[5-(phenylsulfonyl)-2-propylphenyl]sulfonyl}amino)piperidine-1-carboxylate (0.40 g, 77%), as homogeneous, colorless, crystalline solid, m.p. 128-130° C.;

MS (−ESI), m/z: 521.1 [M−H]⁻;

HRMS: calcd for C₂₅H₃₄N₂O₆S₂+NH₄⁺, 540.21965; found (ESI, [M+NH4]⁺), 540.2227;

HPLC purity 100% at 210-370 nm, 10.3 min; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 559

### 5-[(4-fluorophenyl)sulfonyl]-N-(2-phenylethyl)-2-propylbenzenesulfonamide

### Step a) 1-Fluoro-4-[(4-propylphenyl)sulfonyl]benzene

A stirred solution of n-propylbenzene (2.40 g, 20 mmol) and 4-fluorobenzenesulfonyl chloride (3.88 g, 20 mmol) was cooled to −40° C. and treated slowly under nitrogen with solid anhydrous aluminum chloride (3.20 g, 24 mmol). After stirring neat for 4 hours at room temperature, the mixture was poured slowly into ice water and extracted with ethyl acetate (2×). The organic phase was washed with a saturated, aqueous sodium chloride solution, dried over anhydrous sodium sulfate, and filtered through a short column of silica gel. The filtrate was evaporated in vacuo to yield an oil, 1-fluoro-4-[(4-propylphenyl)sulfonyl]benzene (5.56 g, 100%) as a homogeneous, clear, pale, yellow oil;

MS (+ESI), m/z: 279.1 [M+H]⁺;

HRMS: calcd for C₁₅H₁₅FO₂S, 278.07768; found (EI, M+.), 278.0774;

HPLC purity 98.8% at 242 nm, 9.9 min; Xterra RP18, 3.5 u, 150×4.6 mm column, 1.2 ml/min, 85/15-5/95 (Ammon. Form. Buff. Ph=3.5/ACN+MeOH) for 10 min, hold 4 min.

### Step b) 5-[(4-Fluorophenyl)sulfonyl]-2-propylbenzenesulfonyl chloride

1-Fluoro-4-[(4-propylphenyl)sulfonyl]benzene (2.78 g, 10 mmol) was heated with stirring at 60° C. for one hour under nitrogen with chlorosulfonic acid (6.7 mL, 11.7 g, 100 mmol). The mixture was cooled to room temperature, poured slowly into a cold solution of 1N hydrochloric acid, and extracted with ethyl acetate (2×.). The organic phase was washed with a saturated, aqueous sodium chloride solution, dried over anhydrous sodium sulfate, filtered, and the solvent concentrated in vacuo to a homogeneous, clear, tan oil, 5-[(4-fluorophenyl)sulfonyl]-2-propylbenzenesulfonyl chloride (3.74 g, 99%), which solidified on standing and was utilized in subsequent reactions.

### Step c) 5-[(4-Fluorophenyl)sulfonyl]-N-(2-phenylethyl)-2-propylbenzenesulfonamide

A stirred solution of 5-[(4-fluorophenyl)sulfonyl]-2-propylbenzenesulfonyl chloride (0.19 g, 0.5 mmol) in dichloromethane (10 mL) was treated dropwise under nitrogen with a solution of phenethylamine (0.12 g, 1.0 mmol) in dichloromethane. After stirring for one hour at room temperature or until the reaction was complete, the mixture was purified by preparative liquid chromatography on a Biotage® 40 Si column of pre-packed silica gel (45 g), eluting with a gradient of 0%-25% methyl tert-butyl ether in hexane at a flow rate of 50 mL/min. After evaporation of the solvent under vacuum, the title compound, 5-[(4-fluorophenyl)sulfonyl]-N-(2-phenylethyl)-2-propylbenzenesulfonamide (0.22 g, 93%), was obtained as a homogeneous, clear, colorless oil;

MS (−ESI), m/z: 460.1 [M−H]⁻;

HRMS: calcd for C₂₃H₂₄FNO₄S₂+H+, 462.12035; found (ESI, [M+H]⁺), 462.1214;

HPLC purity 100% at 210-370 mn, 10.4 min.; 100% at 242 nm, 10.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 560

### 5-[(4-Fluorophenyl)sulfonyl]-2-propyl-N-(tetrahydro-2H-pyran-4-yl)benzenesulfonamide

The title compound was prepared from 5-[(4-fluorophenyl)sulfonyl]-2-propylbenzenesulfonyl chloride (0.19 g, 0.5 mmol) and tetrahydro-pyran-4-ylamine (0.10 g, 1.0 mmol) according to the procedure and in the same manner as described in Example 559, step c. The crude product was purified by preparative liquid chromatography on a Biotage® 40 Si column of pre-packed silica gel (45 g), eluting with a gradient of 30%-70% methyl tert-butyl ether in hexane at a flow rate of 50 mL/min, to afford 5-[(4-fluorophenyl)sulfonyl]-2-propyl-N-(tetrahydro-2H-pyran-4-yl)benzenesulfonamide (0.18 g, 80%), as a homogeneous, colorless, crystalline solid, m.p. 131-133° C.;

MS (−ESI), m/z: 440.1 [M−H]⁻;

HRMS: calcd for C₂₀H₂₄FNO₅S₂+H+, 442.11527; found (ESI, [M+H]⁺), 442.1644;

HPLC purity 100% at 210-370 nm, 9.2 min; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 561

### 5-[(4-Fluorophenyl)sulfonyl]-2-propyl-N-(tetrahydro-2H-pyran-4-ylmethyl)benzenesulfonamide

The title compound was prepared from 5-[(4-fluorophenyl)sulfonyl]-2-propylbenzenesulfonyl chloride (0.19 g, 0.5 mmol) and (tetrahydro-pyran-4-yl)methylamine (0.12 g, 1.0 mmol) according to the procedure and in the same manner as described in Example 559, step c. The crude product was purified by preparative liquid chromatography on a Biotage® 40 Si column of pre-packed silica gel (45 g), eluting with a gradient of 30%-70% methyl tert-butyl ether in hexane at a flow rate of 50 mL/min, to afford 5-[(4-fluorophenyl)sulfonyl]-2-propyl-N-(tetrahydro-2H-pyran-4-ylmethyl)benzenesulfonamide (0.20 g, 87%), as a glass;

MS (−ESI), m/z: 454.1 [M−H]⁻;

HRMS: calcd for C₂₁H₂₆FNO₅S₂+H+, 456.13092; found (ESI, [M+H]⁺), 299.19;

HPLC purity 100% at 210-370 nm, 9.4 min; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 562

### 5-[(4-Fluorophenyl)sulfonyl]-2-propyl-N-[2-(tetrahydro-2H-pyran-4-yl)ethyl]benzenesulfonamide

The title compound was prepared from 5-[(4-fluorophenyl)sulfonyl]-2-propylbenzenesulfonyl chloride (0.19 g, 0.5 mmol) and 2-(tetrahydro-pyran-4-yl)ethylamine (0.13 g, 1.0 mmol) according to the procedure and in the same manner as described in Example 559, step c. The crude product was purified by preparative liquid chromatography on a Biotage® 40 Si column of pre-packed silica gel (45 g), eluting with a gradient of 30%-70% methyl tert-butyl ether in hexane at a flow rate of 50 mL/min, to afford 5-[(4-fluorophenyl)sulfonyl]-2-propyl-N-[2-(tetrahydro-2H-pyran-4-yl)ethyl]benzenesulfonamide (0.14 g, 58%), as a glass;

MS (−ESI), m/z: 468.1 [M−H]⁻;

HRMS: calcd for C₂₂H₂₈FNO₅S₂+H+, 470.14657; found (ESI, [M+H]⁺), 470.1467;

HPLC purity 100% at 210-370 nm, 9.6 min; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 563

### N-Cyclopentyl-5-[(4-fluorophenyl)sulfonyl]-2-propylbenzenesulfonamide

The title compound was prepared from 5-[(4-fluorophenyl)sulfonyl]-2-propylbenzenesulfonyl chloride (0.19 g, 0.5 mmol) and cyclopentylamine (0.09 g, 1.0 mmol) according to the procedure and in the same manner as described in Example 559, step c. The crude product was purified by preparative liquid chromatography on a Biotage® 40 Si column of pre-packed silica gel (45 g), eluting with a gradient of 0%-25% methyl tert-butyl ether in hexane at a flow rate of 50 mL/min, to afford N-cyclopentyl-5-[(4-fluorophenyl)sulfonyl]-2-propylbenzenesulfonamide (0.15 g, 71%), as a glass;

MS (−ESI), m/z: 424.1 [M−H]⁻;

HRMS: calcd for C₂₀H₂₄FNO₄S₂+H+, 426.12035; found (ESI, [M+H]⁺), 426.1199;

HPLC purity 99.1% at 210-370 nm, 10.2 min; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 564

### 5-[(4-Fluorophenyl)sulfonyl]-2-propyl-N-(2-pyridin-2-ylethyl)benzenesulfonamide

The title compound was prepared from 5-[(4-fluorophenyl)sulfonyl]-2-propylbenzenesulfonyl chloride (0.19 g, 0.5 mmol) and 2-(pyridin-2-yl)ethylamine (0.12 g, 1.0 mmol) according to the procedure and in the same manner as described in Example 559, step c. The crude product was purified by preparative liquid chromatography on a Biotage® 40 Si column of pre-packed silica gel (45 g), eluting with a gradient of 50%-100% methyl tert-butyl ether in hexane at a flow rate of 50 mL/min, to afford 5-[(4-fluorophenyl)sulfonyl]-2-prolpyl-N-(2-pyridin-2-ylethyl)benzenesulfonamide (0.18 g, 78%), as a homogeneous, colorless, crystalline solid, m.p. 113-115° C.;

MS (+ESI), m/z: 463 [M+H]⁺;

MS (−ESI), m/z: 461 [M−H]⁻;

HRMS: calcd for C₂₂H₂₃FN₂O₄S₂+H+, 463.11560; found (ESI, [M+H]⁺), 463.1176;

HPLC purity 100% at 210-370 nm, 9.2 min; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 565

### 5-[(4-Fluorophenyl)sulfonyl]-N-[3-(1H-imidazol-1-yl)propyl]-2-propylbenzenesulfonamide

The title compound was prepared from 5-[(4-fluorophenyl)sulfonyl]-2-propylbenzenesulfonyl chloride (0.38 g, 1.0 mmol) and 3-(1H-imidazol-1yl)propylamine (0.25 g, 2.0 mmol) according to the procedure and in the same manner as described in Example 559, step c. The crude product was purified by preparative liquid chromatography on a Biotage®]40 Si column of pre-packed silica gel (45 g), eluting with a gradient of 0%-4% methanol in dichloromethane at a flow rate of 50 mL/min, to afford 5-[(4-fluorophenyl)sulfonyl]-N-[3-(1H-imidazol-1-yl)propyl]-2-propylbenzenesulfonamide (0.12 g, 25%), as a foam;

MS (−ESI), m/z: 464.1 [M−H]⁻;

HRMS: calcd for C₂₁H₂₄FN₃O₄S₂+H+, 466.12650; found (ESI, [M+H]⁺), 466.125;

HPLC purity 100% at 210-370 nm, 7.6 min; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 566

### 5-[(4-Fluorophenyl)sulfonyl]-2-propylbenzenesulfonamide

The title compound was prepared from 5-[(4-fluorophenyl)sulfonyl]-2-propylbenzenesulfonyl chloride (0.38 g, 1.0 mmol) and 3-(1H-imidazol-1yl)propylamine (0.25 g, 2.0 mmol) according to the procedure and in the same manner as described in Example 559, step c. The crude product was purified by preparative liquid chromatography on a Biotage® 40 Si column of pre-packed silica gel (45 g), eluting with a gradient of 0%-4% methanol in dichloromethane at a flow rate of 50 mL/min, to afford 5-[(4-fluorophenyl)sulfonyl]-2-propylbenzenesulfonamide (0.23 g, 50%), as a tan foam;

MS (−ESI), m/z: 356.1 [M−H]⁻;

HRMS: calcd for C₁₅H₁₆FNO₄S₂+H+, 358.05775; found (ESI, [M+H]⁺), 409.0887;

HPLC purity 79.8% at 210-370 nm, 8.5 min; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 567

### tert-Butyl 4-[({5-[(4-fluorophenyl)sulfonyl]-2-propylphenyl}sulfonyl)amino]piperidine-1-carboxylate

The title compound was prepared from 5-[(4-fluorophenyl)sulfonyl]-2-propylbenzenesulfonyl chloride (0.38 g, 1.0 mmol) and 4-amino-piperidine-1-carboxylic acid, tert-butyl ester (0.40 g, 2.0 mmol) according to the procedure and in the same manner as described in Example 559, step c. The crude product was purified by preparative liquid chromatography on a Biotage® 40 Si column of pre-packed silica gel (45 g), eluting with a gradient of 15%-50% methyl tert-butyl ether in hexane at a flow rate of 50 mL/min, to afford tert-butyl 4-[({5-[(4-fluorophenyl)sulfonyl]-2-propylphenyl}sulfonyl)amino]piperidine-1-carboxylate (0.46 g, 85%), as a homogeneous, colorless, crystalline solid, m.p. 151-153° C.;

MS (−ESI), m/z: 539.1 [M−H]⁻;

HRMS: calcd for C₂₅H₃₃FN₂O₆S₂+NH₄⁺, 558.21023; found (ESI, [M+NH4]⁺), 558.208;

HPLC purity 100% at 210-370 nm, 10.4 min; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 568

### 2-isopropyl-5-{[4-(methylamino)phenyl]sulfonyl}-N-piperidin-4-ylbenzenesulfonamide

100 mg of tert-butyl 4-{[(2-isopropyl-5-{[4-(methylamino)phenyl]sulfonyl}phenyl)sulfonyl]amino}piperidine-1-carboxylate was added to 2 mL of 4M hydrochloric acid in dioxane and the precipitate was filtered giving the desired 2-isopropyl-5-{[4-(methylamino)phenyl]sulfonyl}-N-piperidin-4-ylbenzenesulfonamide.

MS (ES+) m/z 452

MS (ES−) m/z 450

HRMS: calcd. for C₂₁H₂₉N₃O₄S₂+H⁺, 452.167776: found (ESI, [m+H]⁺), 452.166.

### EXAMPLE 569

### 5-[(5-chloro-1,3-dimethyl-1H-pyrazol-4-yl)sulfonyl]-2-isopropyl-N-(tetrahydro-2H-pyran-4-yl)benzenesulfonamide

In an analogous manner to Example 541,

Step 3: 5-bromo-2-isopropyl-N-(tetrahydro-2H-pyran-4-yl)benzenesulfonamide and 5-chloro-1,3-dimethyl-1H-pyrazole-4-sulfonyl fluoride were used to prepare 5-[(5-chloro-1,3-dimethyl-1H-pyrazol-4-yl)sulfonyl]-2-isopropyl-N-(tetrahydro-2H-pyran-4-yl)benzenesulfonamide.

MS (ES) m/z 474.1;

HRMS: calcd for C₁₉H₂₆ClN₃O₅S₂+H+, 476.10752; found (ESI, [M+H]⁺), 476.1059.

### EXAMPLE 570

### N-(2-Phenylethyl)-5-(phenylsulfonyl)-2-(trifluoromethyl)-benzenesulfonamide

Step 1: 1-Benzenesulfonyl-2-nitro-4-trifluomethyl-benzene: A mixture of 4-bromo-3-nitrobenzotrifluoride (10 mL, 65 mmol) and benzenesulfinic acid, sodium salt (10.77 g, 66 mmol) in 100 mL of DMF was stirred under nitrogen overnight at 80° C. After cooling to room temperature the reaction was partitioned with water and methylene chloride. The organic layer was separated and the aqueous layer was extracted three times with methylene chloride. The combined extracts were dried (anhydrous MgSO₄), filtered and the solvent was removed under reduced pressure to give 1-benzenesulfonyl-2-nitro-4-trifluomethyl-benzene (21.52 g, 99%) as a white solid, mp 148-150° C.

Step 2: 2-Benzenesulfonyl-5-trifluoromethyl-phenylamine. A mixture of 1-benzenesulfonyl-2-nitro-4-trifluomethyl-benzene (10.21 g, 31 mmol), prepared in the previous step and Tin(II) chloride (29.22 g, 160 mol) in 100 mL of methanol and 5 mL of water was stirred at reflux under nitrogen for three days. The methanol was removed under reduced pressure and then the residue was partitioned between methylene chloride and 2 N HCl. The organic layer was separated and the aqueous layer extracted three times with methylene chloride. The combined extracts were dried (anhydrous MgSO₄), filtered and the solvent removed under reduced pressure to give 2-benzenesulfonyl-5-trifluoromethyl-phenylamine (8.87 g, 95%) as a white solid, mp 142-143° C.

Step 3: [4-bromo-2-(phenylsulfonyl)-5-(trifluoromethyl)phenyl]amine. A mixture of 2-benzenesulfonyl-5-trifluoromethyl-phenylamine (3.87 g, 13 mmol), prepared in the previous step, benzyltrimethylammonium tribromide (5.02 g, 13 mmol), and calcium carbonate (3.86 g, 40 mmol) in 100 mL methylene chloride and 50 mL of methanol was stirred under nitrogen overnight at room temperature. The solvent was removed under reduced pressure and then the residue was partitioned between methylene chloride and 2 N HCl. The organic layer was separated and the aqueous layer extracted three times with methylene chloride. The combined extracts were dried (anhydrous MgSO₄), filtered and the solvent removed under reduced pressure to produce 4.97 g of a yellow solid. The solid was taken up in methylene chloride and purified on a Horizon™ Flash Collector (Column: Biotage Si 40+M) using a linear gradient of 5% ethyl acetate-hexane to 100% ethyl acetate as the eluent. Isolation of the main component [4-bromo-2-(phenylsulfonyl)-5-(trifluoromethyl)phenyl]amine (4.87g, 94%) as a light yellow solid, mp 169-171° C.; MS (ESI) m/z 380; MS (ESI) m/z 378;

Step 4: 4-Benzenesulfonyl-2-bromo-1-trifluoromethyl-benzene. A solution of Sodium Nitrite (918.9 mg, 13.3 mmol) in 20 mL of water was added under nitrogen at ice bath temperature drop wise over 15 minutes to a solution of [4-bromo-2-(phenylsulfonyl)-5-(trifluoromethyl)phenyl]amine (4.22 g, 11.1 mmol) prepared in the previous step in 200 mL of acetonitrile and 20 mL of concentrated sulfuric acid. After the addition the reaction was stirred at ice bath temperature for thirty minutes. 50% Hypophosphorous acid-water (11.5 mL, 110 mmol) was then added under nitrogen at ice bath temperature drop wise over 20 minutes to the stirring reaction and then was immediately capped and placed into the refrigerator overnight. The reaction was then partitioned between ethyl acetate and water. The ethyl acetate layer was separated, washed four times with water, dried (anhydrous MgSO₄) and the solvent removed under reduced pressure to give 4-benzenesulfonyl-2-bromo-1-trifluoromethyl-benzene (3.20 g, 79%) as a yellow solid.

Step 5: 5-Benzenesulfonyl-2-trifluoromethyl-benzenesulfinic acid. Butyllithium (2.5 M solution in hexanes; 1.6 mL, 4.0 mmol) was added under nitrogen over approximately two minutes to a solution of 4-benzenesulfonyl-2-bromo-1-trifluoromethyl-benzene (1.0 g, 2.7 mmol) in 10 mL of anhydrous tetrahydrofuran at dry ice-acetone temperature. After the addition the reaction was stirred at dry ice-acetone temperature for one hour. After that time SO₂ gas was bubbled into the reaction flask for approximately five minutes at dry ice-acetone bath temperature. After warming to room temperature the solvent and the excess SO₂ were removed under reduced pressure and then the residue was partitioned between methylene chloride and 2 N HCl. The organic layer was separated and the aqueous layer extracted three times with methylene chloride. The combined extracts were dried (anhydrous MgSO₄), filtered and the solvent removed under reduced pressure to produce 0.77 g of brown solid.

Step 6: N-(2-phenylethyl)-5-(phenylsulfonyl)-2-(trifluoromethyl)benzenesulfonamide. A mixture of 5-benzenesulfonyl-2-trifluoromethyl-benzenesulfinic acid (0.58 g, 1.6 mmol), prepared in the previous step, N-Chlorosuccinimide (0.26 g, 1.98 mmol), and triethylamine (0.7 mL, 5 mmol) in 30 mL methylene chloride was stirred under nitrogen for thirty minutes at room temperature. Phenethylamine (0.25 mL, 2 mmol) was slowly syringed into the flask and the reaction was stirred under nitrogen overnight at room temperature. The solvent was removed under reduced pressure and then the residue was partitioned between methylene chloride and 2 N HCl. The organic layer was separated and the aqueous layer extracted three times with methylene chloride. The combined extracts were dried (anhydrous MgSO₄), filtered and the solvent removed under reduced pressure to produce 0.82 g of brown solid. The solid was taken up in methylene chloride and purified on a Horizon™ Flash Collector (Column: Biotage Si 25+M) using a linear gradient of 5% methylene chloride-hexane to 100% methylene chloride as the eluent. Additional purification was done on 0.20 g of product with a Luna C18 (50×250 mm) reverse phase column on a Varian HPLC system using 70% methanol/water mobile phase isocraticly at 100 ml/min. Isolation of the main component gave the title compound N-(2-phenylethyl)-5-(phenylsulfonyl)-2-(trifluoromethyl)benzenesulfonamide (0.23 g, 35.4%), as a white solid mp 151-153° C.

MS (ES) m/z 468.1.

### EXAMPLE 571

### 5-(Phenylsulfonyl)-N-(2-pyridin-2-ylethyl)-2-(trifluoromethyl)benzenesulfonamide

A mixture of 5-benzenesulfonyl-2-trifluoromethyl-benzenesulfinic acid (0.59 g, 1.7 mmol), prepared in the same manner as described in step 5 of Example 570, N-Chlorosuccinimide (0.27 g, 2.0 mmol), and triethylamine (0.7 mL, 5 mmol) in 30 ml methylene chloride was stirred under nitrogen for thirty minutes at room temperature. 2-(2-pyridyl)ethylamine (0.24 mL, 2 mmol) was slowly syringed into the flask and the reaction was stirred under nitrogen overnight at room temperature. The solvent was removed under reduced pressure and then the residue was partitioned between methylene chloride and 2 N HCl. The organic layer was separated and the aqueous layer extracted three times with methylene chloride. The combined extracts were dried (anhydrous MgSO₄), filtered and the solvent was removed under reduced pressure to produce 0.67 g of a dark brown solid. The solid was taken up in methylene chloride and purified on a Horizon™ Flash Collector (Column: Biotage Si 25+M) using a linear gradient of 5% methylene chloride-hexane to 100% methylene chloride as the eluent. Isolation of the main component gave the title compound 5-(phenylsulfonyl)-N-(2-pyridin-2-ylethyl)-2-(trifluoromethyl)benzenesulfonamide (75.5 mg, 9.6%) as a white solid, mp 149-151° C.;

MS (ES) m/z 469.0.

### EXAMPLE 572

### 5-[(4-fluorophenyl)sulfonyl]-2-isopropyl-N-[1-(morpholin-4-ylcarbonyl)piperidin-4-yl]benzenesulfonamide

In an analogous manner to Example 462, 2-isopropyl-5-(phenylsulfonyl)-n-piperidin-4-ylbenzenesulfonamide and 4-morpholinecarbonyl chloride were used to prepare 5-[(4-fluorophenyl)sulfonyl]-2-isopropyl-N-[1-(morpholin-4-ylcarbonyl)piperidin-4-yl]benzenesulfonamide.

MS (ES+) m/z 554

MS (ES−) m/z 552

HRMS: calcd. for C₂₅H₃₂FN₃O₆S₂+H⁺, 554.179484: found (ESI, [m+H]⁺), 554.1799.

### EXAMPLE 573

### 4-[({5-[(4-fluorophenyl)sulfonyl]-2-isopropylphenyl}sulfonyl)amino]-N,N-dimethylpiperidine-1-carboxamide

In an analogous manner to Example 462, 2-isopropyl-5-(phenylsulfonyl)-n-piperidin-4-ylbenzenesulfonamide and dimethylcarbamyl chloride were used to prepare 4-[({5-[(4-fluorophenyl)sulfonyl]-2-isopropylphenyl}sulfonyl)amino]-N,N-dimethylpiperidine-1-carboxamide.

MS (ES+) m/z 512

MS (ES−) m/z 510

HRMS: calcd. for C₂₃H₃₀FN₃O₅S₂+H⁺, 512.168919: found (ESI, [m+H]⁺), 512.1692.

### EXAMPLE 574

### N-(1-benzylpiperidin-4-yl)-5-[(4-fluorophenyl)sulfonyl]-2-methylbenzenesulfonamide

In an analogous manner to Example 435, 5-(4-fluoro-benzenesulfonyl)-2-methyl-benzenesulfonyl chloride and 4-amino-1-benzylpiperidine were used to prepare N-(1-benzylpiperidin-4-yl)-5-[(4-fluorophenyl)sulfonyl]-2-methylbenzenesulfonamide.

MS (ES+) m/z 503

MS (ES−) m/z 501

HRMS: calcd. for C₂₅H₂₇FN₂O₄S₂+H⁺, 503.147455: found (ESI, [m+H]⁺), 503.1489.

### EXAMPLE 575

### 2-isopropyl-5-(phenylsulfinyl)-N-(2-pyridin-2-ylethyl)benzenesulfonamide

Step 1: Following the same procedure as described in Example 474 (Step 1), 2-bromoisopropylbenzene was used to prepare 2-isopropyl-N-(2-pyridin-2-ylethyl)benzenesulfonamide.

Step 2: Following the same procedure as described in Example 474 (Step 2), 2-isopropyl-N-(2-pyridin-2-ylethyl)benzenesulfonamide was used to prepare 5-bromo-2-isopropyl-N-(2-pyridin-2-ylethyl)benzenesulfonamide.

Step 3: Following the same procedure as described in Example 346, 5-bromo-2-isopropyl-N-(2-pyridin-2-ylethyl)benzenesulfonamide, and benzenethiol were used to prepare 2-isopropyl-5-(phenylsulfinyl)-N-(2-pyridin-2-ylethyl)benzenesulfonamide.

MS (ES) m/z 427.1;

HRMS: calcd for C₂₂H₂₄N₂O₃S₂+H+, 429.1301 1; found (ESI, [M+H]⁺), 429.1309.

### EXAMPLE 576

### 5-[(4-fluorophenyl)sulfonyl]-2-methyl-N-piperidin-4-ylbenzenesulfonamide

200 mg of palladium on carbon were wetted with 30 mL of ethanol and 1.5 g of N-(1-benzylpiperidin-4-yl)-5-[(4-fluorophenyl)sulfonyl]-2-methylbenzenesulfonamide hydrochloride was added. The reaction was mixed on a Parr shaker at 45 psi overnight. The reaction mixture was filtered through Celite® and the filtrate was concentrated down giving the desired 5-[(4-fluorophenyl)sulfonyl]-2-methyl-N-piperidin-4-ylbenzenesulfonamide.

MS (ES+) m/z 413

MS (ES−) m/z 411

HRMS: calcd. for C₁₈H₂₁FN₂O₄S₂+H⁺, 413.100505: found (ESI, [m+H]⁺), 413.0997.

### EXAMPLE 577

### 5-[(3-bromo-2-methylphenyl)sulfonyl]-2-isopropyl-N-(2-pyridin-2-ylethyl)benzenesulfonamide

Step 1: Following the same procedure described in Example 334, 2-methyl-3-bromoaniline was used to prepare 2-methyl-3-bromobenzenesulfonyl chloride.

Step 2: Following the same procedure described in Example 474, 2-methyl-3-bromobenzenesulfonyl chloride was used to prepare 2-methyl-3-bromobenzenesulfonyl fluoride.

Step 4: Following the same procedure described in Example 474, 5-bromo-2-isopropyl-N-(2-pyridin-2-ylethyl)benzenesulfonamide and 3-bromo-2-methylbenzene sulfonyl fluoride were used to prepare 5-[(3-bromo-2-methylphenyl)sulfonyl]-2-isopropyl-N-(2-pyridin-2-ylethyl)benzenesulfonamide.

MS (ESI) m/z 537;

HRMS: calcd for C₂₃H₂₅BrN₂O₄S₂+H+, 537.05119; found (ESI, M+H), 537.0517.

### EXAMPLE 578

### 5-[(2-chloro-6-methylphenyl)sulfonyl]-2-isopropyl-N-(2-pyridin-2-ylethyl)benzenesulfonamide

Step 1: Following the same procedure described in Example 474 (Step 3), 2-chloro-6-methylbenzene sulfonyl chloride was used to prepare 2-chloro-6-methylbenzene sulfonyl fluoride.

Step 2: Following the same procedure described in Example 474 (Step 4), 5-bromo-2-isopropyl-N-(2-pyridin-2-ylethyl)benzenesulfonamide and 2-chloro-6-methylbenzene sulfonyl fluoride were used to prepare 5-[(2-chloro-6-methylphenyl)sulfonyl]-2-isopropyl-N-(2-pyridin-2-ylethyl)benzenesulfonamide.

MS (ESI) m/z 493;

HRMS: calcd for C₂₃H₂₅ClN₂O₄S₂+H+, 493.10170; found (ESI, M+H), 493.1023.

### EXAMPLE 579

### 2-isopropyl-5-[(3-methylphenyl)sulfonyl]-N-(2-pyridin-2-ylethyl)benzenesulfonamide

Step 1: Following the same procedure described in Example 474 (Step 3), 3-methylbenzene sulfonyl chloride was used to prepare 3-methylbenzene sulfonyl fluoride.

Step 2: Following the same procedure described in Example 474 (Step 4), 5-bromo-2-isopropyl-N-(2-pyridin-2-ylethyl)benzenesulfonamide and 3-methylbenzene sulfonyl fluoride were used to prepare 2-isopropyl-5-[(3-methylphenyl)sulfonyl]-N-(2-pyridin-2-ylethyl)benzenesulfonamide.

MS (ESI) m/z 459;

HRMS: calcd for C₂₃H₂₆N₂O₄S₂+H+, 459.14067; found (ESI, M+H), 459.1412.

### EXAMPLE 580

### 5-({4-[(2-cyanoethyl)(methyl)amino]phenyl}sulfonyl)-2-isopropyl-N-(tetrahydro-2H-pyran-4-yl)benzenesulfonamide

In an analogous manner to Example 353, 5-[(4-fluorophenyl)sulfonyl]-2-isopropyl-N-tetrahydro-2H-pyran-4-ylbenzenesulfonamide and 3-(methylamino)propionitrile in DMA were used to prepare 5-({4-[(2-cyanoethyl)(methyl)amino]phenyl}sulfonyl)-2-isopropyl-N-(tetrahydro-2H-pyran-4-yl)benzenesulfonamide.

MS (ESI) m/z 506;

HPLC purity 96.0% at 210-370 nm, 8.6 min.; 95.8% at 312 nm, 8.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₄H₃₁N₃O₅S₂+H+, 506.17779; found (ESI, [M+H]+), 506.1783.

### EXAMPLE 581

### 5-({4-[(2-cyanoethyl)(methyl)amino]phenyl}sulfonyl)-2-isopropyl-N-(tetrahydro-2H-pyran-4-ylmethyl)benzenesulfonamide

In an analogous manner to Example 353, 5-[(4-fluorophenyl)sulfonyl]-2-isopropyl-N-tetrahydro-2H-pyran-4-ylmethyl)benzenesulfonamide and 3-(methylamino)propionitrile in DMA were used to prepare 5-({4-[(2-cyanoethyl)(methyl)amino]phenyl}sulfonyl)-2-isopropyl-N-(tetrahydro-2H-pyran-4-ylmethyl)benzenesulfonamide.

MS (ESI) m/z 520;

HPLC purity 96.3% at 210-370 nm, 8.8 min.; 96.0% at 312 nm, 8.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₅H₃₃N₃O₅S₂+H+, 520.19344; found (ESI, [M+H]⁺), 520.194.

### EXAMPLE 582

### 5-[(4-fluorophenyl)sulfonyl]-N-(2-hydroxyethyl)-2-isopropylbenzenesulfonamide

In an analogous manner to Example 230:

Step 3: 2-Isopropyl-5-(4-fluoro-benzenesulfonyl)-benzenesulfonyl chloride and 2-aminoethanol were used to prepare 5-[(4-fluorophenyl)sulfonyl]-N-(2-hydroxyethyl)-2-isopropylbenzenesulfonamide.

MS (ESI+) m/z 402;

MS (ESI−) m/z 400;

HPLC purity 100% at 210-370 nm, 8.3 min.; 100% at 242 nm, 8.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₇H₂₀FNO₅S₂+H+, 402.08397; found (ESI, [M+H]+), 402.0845.

### EXAMPLE 583

### 5-[(4-fluorophenyl)sulfonyl]-2-isopropyl-N-[2-(2-methyl-1H-imidazol-1-yl)ethyl]benzenesulfonamide

Step 1: To a stirred solution of 5-[(4-fluorophenyl)sulfonyl]-N-(2-hydroxyethyl)-2-isopropylbenzenesulfonamide (0.6 mg, 1.5 mmol), triethylamine (2.2 mmol) and a catalytic amount of N,Ndimethylaminopyridine in dichloromethane was added 4-methylbenzenesulfonyl chloride (1.8 mmol) and after 4 hours an additional 1.8 mmol of 4-methylbenzene sulfonyl chloride was added. The reaction was concentrated and then purified by flash column chromatography using an ethyl acetate-hexane gradient affording predominately the N,O-bistosyl intermediate.

Step 2: 2-Methylimidazole (164 mg, 2 mmol) was dissolved in anhydrous tetrahydrofuran (1 mL) and sodium hydride (60% mineral oil dispersion, 24 mg, 1 mmol) was added. After 30 minutes, a solution of the intermediate bistosylate (step 1, 0.14 mmol) in tetrahydrofuran was added and the reaction was allowed to stir at room temperature for 30 additional minutes. The mixture was quenched upon the addition of water. The mixture was extracted with ethyl acetate (3×2 mL). The combined organic phases were concentrated and the resulting crude residue was purified by automated flash column chromatography resulting in the recovery of 56 mg (85%) of 5-[(4-fluorophenyl)sulfonyl]-2-isopropyl-N-[2-(2-methyl-1H-imidazol-1-yl)ethyl]benzenesulfonamide.

MS (ESI+) m/z 466;

MS (ESI−) m/z 464;

HPLC purity 100% at 210-370 nm, 7.5 min.; 100% at 244 nm, 7.5 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₁H₂₄FN₃O₄S₂+H+, 466.12650; found (ESI, [M+H]+), 466.1271.

### EXAMPLE 584

### N-[2-(2-ethyl-1H-imidazol-1-yl)ethyl]-5-[(4-fluorophenyl)sulfonyl]-2-isopropylbenzenesulfonamide

In an analogous manner to Example 583:

Step 2: 2-The bistosylate and 2-ethylimidazole were used to prepare N-[2-(2-ethyl-1H-imidazol-1-yl)ethyl]-5-[(4-fluorophenyl)sulfonyl]-2-isopropylbenzenesulfonamide.

MS (ESI+) m/z 480;

MS (ESI−) m/z 478;

HPLC purity 100% at 210-370 nm, 7.7 min.; 100% at 244 nm, 7.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₂H₂₆FN₃O₄S₂+H+, 480.14215; found (ESI, [M+H]+), 480.1427.

### EXAMPLE 585

### N-(1-acetylpiperidin-4-yl)-5-[(4-fluorophenyl)sulfonyl]-2-methylbenzenesulfonamide

In an analogous manner to Example 462, 5-[(4-fluorophenyl)sulfonyl]-2-methyl-N-piperidin-4-ylbenzenesulfonamide and acetic anhydride were used to prepare N-(1-acetylpiperidin-4-yl)-5-[(4-fluorophenyl)sulfonyl]-2-methylbenzenesulfonamide.

MS (ES+) m/z 455

MS (ES−) m/z 453.

### EXAMPLE 586

### N-[1-(4-cyanobenzoyl)piperidin-4-yl]-5-[(4-fluorophenyl)sulfonyl]-2-methylbenzenesulfonamide

In an analogous manner to Example 462, 5-[(4-fluorophenyl)sulfonyl]-2-methyl-N-piperidin-4-ylbenzenesulfonamide and 4-cyanobenzoyl chloride were used to prepare N-[1-(4-cyanobenzoyl)piperidin-4-yl]-5-[(4-fluorophenyl)sulfonyl]-2-methylbenzenesulfonamide.

MS (ES+) m/z 542

MS (ES−) m/z 540.

### EXAMPLE 587

### 4-[({5-[(4-fluorophenyl)sulfonyl]-2-methylphenyl}sulfonyl)amino]-N,N-dimethylpiperidine-1-carboxamide

In an analogous manner to Example 462, 5-[(4-fluorophenyl)sulfonyl]-2-methyl-N-piperidin-4-ylbenzenesulfonamide and dimethylcarbamyl chloride were used to prepare 4-[({5-[(4-fluorophenyl)sulfonyl]-2-methylphenyl}sulfonyl)amino]-N,N-dimethylpiperidine-1-carboxamide.

MS (ES+) m/z 484

MS (ES−) m/z 482.

### EXAMPLE 588

### 5-[(4-fluorophenyl)sulfonyl]-2-isopropyl-N-[2-(2-isopropyl-1H-imidazol-1-yl)ethyl]benzenesulfonamide

In an analogous manner to Example 583:

Step 2: 2—The bistosylate and 2-isopropylimidazole were used to prepare 5-[(4-fluorophenyl)sulfonyl]-2-isopropyl-N-[2-(2-isopropyl-1H-imidazol-1-yl)ethyl]benzenesulfonamide.

MS (ESI+) m/z 494;

MS (ESI−) m/z 492;

HPLC purity 91.6% at 210-370 nm, 8.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₃H₂₈FN₃O₄S₂+H+, 494.15780; found (ESI, M+H), 494.1584.

### EXAMPLE 589

### 5-[(4-fluorophenyl)sulfonyl]-N-[1-(methoxyacetyl)piperidin-4-yl]-2-methylbenzenesulfonamide

In an analogous manner to Example 462, 5-[(4-fluorophenyl)sulfonyl]-2-methyl-N-piperidin-4-ylbenzenesulfonamide and methoxyacetyl chloride were used to prepare 5-[(4-fluorophenyl)sulfonyl]-N-[1-(methoxyacetyl)piperidin-4-yl]-2-methylbenzenesulfonamide.

MS (ES+) m/z 485

MS (ES−) m/z 483

HRMS: calcd. for C₂₁H₂₅FN₂O₆S₂+H⁺, 485.121634: found (ESI, [m+H]⁺), 485.1216.

### EXAMPLE 590

### 2-isopropyl-5-[(R)-phenylsulfinyl]-N-(2-pyridin-2-ylethyl)benzenesulfonamide

Chiral separation of 2-isopropyl-5-(phenylsulfinyl)-N-(2-pyridin-2-ylethyl)benzenesulfonamide using a prep chiral SFC column AD-H 35% methanol/65% CO₂ at 50 mL/min yielded 2-isopropyl-5-[(R)-phenylsulfinyl]-N-(2-pyridin-2-ylethyl)benzenesulfonamide.

MS (ES) m/z 426.9.

### EXAMPLE 591

### 2-isopropyl-5-[(S)-phenylsulfinyl]-N-(2-pyridin-2-ylethyl)benzenesulfonamide

Chiral separation of 2-isopropyl-5-(phenylsulfinyl)-N-(2-pyridin-2-ylethyl)benzenesulfonamide using a prep chiral SFC column AD-H 35% methanol/65% CO₂ at 50 mL/min yielded 2-isopropyl-5-[(S)-phenylsulfinyl]-N-(2-pyridin-2-ylethyl)benzenesulfonamide.

MS (ES) m/z 426.9.

### EXAMPLE 592

### 2-bromo-5-(phenylsulfonyl)-N-(2-pyridin-2-ylethyl)benzenesulfonamide

Step 1: Following the same procedure described in Example 1 (Step 1), 4-nitrobromobenzene and chlorosulfonic acid were used to prepare 2-bromo-5-nitrobenzenesulfonyl chloride.

Step 2: Following the same procedure described in Example 1 (Step 2), 2-(2-ethylamino)pyridine and 2-bromo-5-nitrobenzenesulfonyl chloride were used to prepare 2-bromo-5-nitro-N-(2-pyridin-2-ylethyl)benzenesulfonamide.

Step 3: 2-bromo-5-nitro-N-(2-pyridin-2-ylethyl)benzenesulfonamide (1.5 g, 3.88 mmol) was dissolved in methanol (25 mL) and water (1 mL). Tin (II) chloride (3.68 g, 19.4 mmol) was added and the resulting solution was heated to 70° C. overnight. The solution was concentrated and partitioned between ethyl acetate and a saturated aqueous sodium bicarbonate solution. The organic layer was dried over magnesium sulfate and concentrated to give 2-bromo-5-amino-N-(2-pyridin-2-ylethyl)benzenesulfonamide (1.2 g, 87%).

Step 4: Following the same procedure described in Example 334 (Step 2), 2-bromo-5-amino-N-(2-pyridin-2-ylethyl)benzenesulfonamide was used to prepare 4-bromo-3-(2-pyridin-2-yl-ethylsulfamoyl)-benzenesulfonyl chloride.

Step 5: To a stirred solution of aluminum chloride (1.82 g, 13.64 mmol) in nitrobenzene (40 mL), was added benzene (3 mL), and 4-bromo-3-(2-pyridin-2-yl-ethylsulfamoyl)-benzenesulfonyl chloride (1.0 g, 2.27 mmol) at 80° C. The resulting mixture was stirred 4 days at 80° C. with daily addition of benzene (2 mL). The reaction was then cooled to room temperature, poured over ice and extracted with dichloromethane. The organic phase was concentrated and flash column separated with 50% ethyl acetate/hexane to give 2-bromo-5-(phenylsulfonyl)-N-(2-pyridin-2-ylethyl)benzenesulfonamide (0.23 g, 21%).

MS (ES) m/z 478.8;

HRMS: calcd for C₁₉H₁₇BrN₂O₄S₂+H+, 480.98859; found (ESI, M+H), 480.9891.

### EXAMPLE 593

### 5-[(3-cyano-2-methylphenyl)sulfonyl]-2-isopropyl-N-(2-pyridin-2-ylethyl)benzenesulfonamide

In an analogous manner to Example 376, 5-[(3-bromo-2-methylphenyl)sulfonyl]-2-isopropyl-N-(2-pyridin-2-ylethyl)benzenesulfonamide was used to prepare 5-[(3-cyano-2-methylphenyl)sulfonyl]-2-isopropyl-N-(2-pyridin-2-ylethyl)benzenesulfonamide.

MS (ES) m/z 481.9.

### EXAMPLE 594

### 5-[(3-acetyl-2-methylphenyl)sulfonyl]-2-isopropyl-N-(2-pyridin-2-ylethyl)benzenesulfonamide

In an analogous manner to Example 380, 5-[(3-bromo-2-methylphenyl)sulfonyl]-2-isopropyl-N-(2-pyridin-2-ylethyl)benzenesulfonamide was used to prepare 5-[(3-acetyl-2-methylphenyl)sulfonyl]-2-isopropyl-N-(2-pyridin-2-ylethyl)benzenesulfonamide.

MS (ES) m/z 499.0;

HRMS: calcd for C₂₅H₂₈N₂O₅S₂+H+, 501.15124; found (ESI, M+H), 501.1518.

### EXAMPLE 595

### 5-[(3-chloro-4-fluorophenyl)sulfonyl]-2-isopropyl-N-(2-pyridin-2-ylethyl)benzenesulfonamide

Step 1: Following the same procedure described in Example 474 (Step 3), 3-chloro-4-fluorobenzene sulfonyl fluoride was used to prepare 3-chloro-4-fluorobenzene sulfonyl fluoride.

Step 2: Following the same procedure described in Example 474 (Step 4), 5-bromo-2-isopropyl-N-(2-pyridin-2-ylethyl)benzenesulfonamide and 3-chloro-4-fluorobenzene sulfonyl fluoride were used to prepare 5-[(3-chloro-4-fluorophenyl)sulfonyl]-2-isopropyl-N-(2-pyridin-2-ylethyl)benzenesulfonamide.

MS (ESI) m/z 497;

MS (ESI) m/z 495.

### EXAMPLE 596

### 5-[(4-fluoro-2-methylphenyl)sulfonyl]-2-isopropyl-N-(2-pyridin-2-ylethyl)benzenesulfonamide

Step 1: Following the same procedure described in Example 474 (Step 3), 2-methyl-4-fluorobenzene sulfonyl chloride was used to prepare 2-methyl-4-fluorobenzene sulfonyl fluoride.

Step 2: Following the same procedure described in Example 474 (Step 4), 5-bromo-2-isopropyl-N-(2-pyridin-2-ylethyl)benzenesulfonamide and 2-methyl-4-fluorobenzene sulfonyl fluoride were used to prepare 5-[(4-fluoro-2-methylphenyl)sulfonyl]-2-isopropyl-N-(2-pyridin-2-ylethyl)benzenesulfonamide.

MS (ES) m/z 474.9;

HRMS: calcd for C₂₃H₂₅FN₂O₄S₂+H+, 477.13125; found (ESI, M+H), 477.1318.

### EXAMPLE 597

### 2-isopropyl-5-[(1-methyl-1H-indol-5-yl)sulfonyl]-N-(2-pyridin-2-ylethyl)benzenesulfonamide

Step 1: To a stirred solution of 5-bromoindole (2.0 g, 10.2 mmol) in DMF (25 mL) at 0° C. was added sodium hydride (0.45 g, 60%, 11.22 mmol) and the resulting solution was stirred for 5 minutes. Methyl iodide (1.3 mL, 20.8 mmol) was added and the reaction was allowed to warm to room temperature. After 1 hr the reaction was quenched with a saturated aqueous ammonium chloride solution and extracted several times with ethyl acetate. The combined organic layers were washed with brine, dried over magnesium sulfate, and concentrated to give crude 1-methyl-5-bromoindole. This material was dissolved in THF (20 mL) and chilled to −78° C. n-Butyl lithium 2.5 M in hexane (3.8 mL, 9.5 mmol) was added dropwise. After 10 minutes sulfur dioxide was bubbled into the reaction mixture for 5 minutes. The mixture was allowed to warm to room temperature and concentrated. The crude mixture was taken up in methylene chloride (20 mL) and N-bromosuccinimide (1.25 g, 9.36 mmol) was added and the resulting solution was stirred for 1 hr. The solution was washed with a saturated aqueous ammonium chloride solution, dried over magnesium sulfate, and concentrated. The mixture was flash column separated using 20% ethyl acetate/hexane to give 1-methylindole-5-sulfonyl chloride (0.64 g, 27%).

Step 2: Following the same procedure described in Example 474 (Step 3), 1-methylindole-5-sulfonyl chloride was used to prepare 1-methylindole-5-sulfonyl fluoride.

Step 3: Following the same procedure described in Example 474 (Step 4), 5-bromo-2-isopropyl-N-(2-pyridin-2-ylethyl)benzenesulfonamide and 1-methylindole-5-sulfonyl fluoride were used to prepare 5-[(4-fluoro-2-methylphenyl)sulfonyl]-2-isopropyl-N-(2-pyridin-2-ylethyl)benzenesulfonamide.

MS (ESI) m/z 498;

MS (ESI) m/z 496.

### EXAMPLE 598

### 2-isopropyl-5-[(3-methoxyphenyl)sulfonyl]-N-(tetrahydro-2H-pyran-4-yl)benzenesulfonamide

Step 1: Following the same procedure described in Example 474 (Step 3), 3-methoxybenzene sulfonyl chloride was used to prepare 3-methoxybenzenesulfonyl fluoride.

Step 2: 3-methoxybenzenesulfonyl fluoride and 5-bromo-2-isopropyl-N-(tetrahydro-2H-pyran-4-yl)benzenesulfonamide were used to prepare 2-isopropyl-5-[(3-methoxyphenyl)sulfonyl]-N-(tetrahydro-2H-pyran-4-yl)benzenesulfonamide.

MS (ESI) m/z 454;

MS (ESI) m/z 452.

### EXAMPLE 599

### 2-isopropyl-5-{[2-methyl-4-(methylamino)phenyl]sulfonyl}-N-(2-pyridin-2-ylethyl)benzenesulfonamide

In an analogous manner to Example 356, 5-[(4-fluoro-2-methylphenyl)sulfonyl]-2-isopropyl-N-(2-pyridin-2-ylethyl)benzenesulfonamide was used to prepare 2-isopropyl-5-{[2-methyl-4-(methylamino)phenyl]sulfonyl}-N-(2-pyridin-2-ylethyl)benzenesulfonamide.

MS (ESI) m/z 488;

MS (ESI) m/z 486.

### EXAMPLE 600

### 5-{[4-(dimethylamino)-2-methylphenyl]sulfonyl}-2-isopropyl-N-(2-pyridin-2-ylethyl)benzenesulfonamide

In an analogous manner to Example 353, 5-[(4-fluoro-2-methylphenyl)sulfonyl]-2-isopropyl-N-(2-pyridin-2-ylethyl)benzenesulfonamide was used to prepare 5-{[4-(dimethylamino)-2-methylphenyl]sulfonyl}-2-isopropyl-N-(2-pyridin-2-ylethyl)benzenesulfonamide.

MS (ES) m/z 500.1;

HRMS: calcd for C₂₅H₃₁N₃O₄S₂+H+, 502.18287; found (ESI-FTMS, [M+H]¹⁺), 502.18264.

### EXAMPLE 601

### 2-(dimethylamino)-5-(phenylsulfonyl)-N-(2-pyridin-2-ylethyl)benzenesulfonamide

Step 1: Following the same procedure described in Example 1 (Step 1), 4-nitrobromobenzene and chlorosulfonic acid were used to prepare 2-bromo-5-nitrobenzenesulfonyl chloride.

Step 2: Following the same procedure described in Example 1 (Step 2), 2-(2-ethylamino)pyridine and 2-bromo-5-nitrobenzenesulfonyl chloride were used to prepare 2-bromo-5-nitro-N-(2-pyridin-2-ylethyl)benzenesulfonamide.

Step 3: 2-bromo-5-nitro-N-(2-pyridin-2-ylethyl)benzenesulfonamide (1.5 g, 3.88 mmol) was dissolved in dimethylamine 2.0 M in THF (10 mL) and the resulting solution was microwave heated for 10 mintues at 160° C. The solution was diluted with ethyl acetate and washed with a saturated aquous ammonium chloride solution. The organic layer was dried over magnesium sulfate, filtered, and concentrated to give 2-dimethylamino-5-nitro-N-(2-pyridin-2-ylethyl)benzenesulfonamide (1.3 g, 95%).

Step 4: Following the same procedure described in Example 592 (Step 3), 2-dimethylamino-5-nitro-N-(2-pyridin-2-ylethyl)benzenesulfonamide was used to prepare 2-dimethylamino-5-amino-N-(2-pyridin-2-ylethyl)benzenesulfonamide.

Step 5: To a stirred solution of 2-dimethylamino-5-amino-N-(2-pyridin-2-ylethyl)benzenesulfonamide (0.60 g, 1.87 mmol) in acetonitrile (15 mL) and hydrobromic acid 48% (1.5 mL) at 0° C. was added sodium nitrite (0.16 g, 2.2 mmol) dissolved in a minimum amount of water dropwise. The resulting solution was stirred 20 minutes and copper I bromide (0.36 g, 2.5 mmol) was added and stirred 1 hr. The solution was diluted with water, neutralized with sodium hydroxide and extracted with ethyl acetate. The organic layer was dried over magnesium sulfate, filtered, and concentrated to give 2-dimethylamino-5-bromo-N-(2-pyridin-2-ylethyl)benzenesulfonamide (0.49 g, 81%).

Step 6: Following the same procedure described in Example 474 (Step 4), 2-dimethylamino-5-bromo-N-(2-pyridin-2-ylethyl)benzenesulfonamide and benzene sulfonyl fluoride were used to prepare 2-(dimethylamino)-5-(phenylsulfonyl)-N-(2-pyridin-2-ylethyl)benzenesulfonamide.

MS (ES) m/z 444.1;

HRMS: calcd for C₂₁H₂₃N₃O₄S₂+H+, 446.12027; found (ESI-FTMS, [M+H]¹⁺), 446.1205

### EXAMPLE 602

### N-[1-(cyclopropylcarbonyl)piperidin-4-yl]-N-({5-[(4-fluorophenyl)sulfonyl]-2-methylphenyl}sulfonyl)cyclopropanecarboxamide

In an analogous manner to Example 462, 5-[(4-fluorophenyl)sulfonyl]-2-methyl-N-piperidin-4-ylbenzenesulfonamide and cyclopropylcarbonyl chloride were used to prepare N-[1-(cyclopropylcarbonyl)piperidin-4-yl]-N-({5-[(4-fluorophenyl)sulfonyl]-2-methylphenyl}sulfonyl)cyclopropanecarboxamide.

MS (ES+) m/z 549.

### EXAMPLE 603

### 5-[(4-fluorophenyl)sulfonyl]-2-methyl-N-[1-(morpholin-4-ylcarbonyl)piperidin-4-yl]benzenesulfonamide

In an analogous manner to Example 462, 5-[(4-fluorophenyl)sulfonyl]-2-methyl-N-piperidin-4-ylbenzenesulfonamide and 4-morpholinecarbonyl chloride were used to prepare 5-[(4-fluorophenyl)sulfonyl]-2-methyl-N-[1-(morpholin-4-ylcarbonyl)piperidin-4-yl]benzenesulfonamide.

MS (ES+) m/z 526

MS (ES−) m/z 524.

### EXAMPLE 604

### 2-chloro-5-[(3-methylphenyl)sulfonyl]-N-(tetrahydro-2H-pyran-4-yl)benzenesulfonamide

Step 1: Following the same procedure described in Example 1 (Step 2), 4-aminotetrahydropyran and 2-chloro-5-nitrobenzenesulfonyl chloride were used to prepare 5-nitro-2-chloro-N-(tetrahydro-2H-pyran-4-yl)benzenesulfonamide.

Step 2: Following the same procedure described in Example 592 (Step 3), 5-nitro-2-chloro-N-(tetrahydro-2H-pyran-4-yl)benzenesulfonamide was used to prepare 5-amino-2-chloro-N-(tetrahydro-2H-pyran-4-yl)benzenesulfonamide.

Step 3: Following the same procedure described in Example 601 (Step 5), 5-amino-2-chloro-N-(tetrahydro-2H-pyran-4-yl)benzenesulfonamide was used to prepare 5-bromo-2-chloro-N-(tetrahydro-2H-pyran-4-yl)benzenesulfonamide.

Step 4: Following the same procedure described in Example 474 (Step 3), 3-methylbenzene sulfonyl chloride was used to prepare 3-methylbenzenesulfonyl fluoride.

Step 5: Following the same procedure described in Example 474 (Step 4), 5-bromo-2-chloro-N-(tetrahydro-2H-pyran-4-yl)benzenesulfonamide and 3-methylbenzene sulfonyl fluoride were used to prepare 2-chloro-5-[(3-methylphenyl)sulfonyl]-N-(tetrahydro-2H-pyran-4-yl)benzenesulfonamide.

MS (ES) m/z 428.0.

### EXAMPLE 605

### 5-{[3-chloro-4-(methylamino)phenyl]sulfonyl}-2-isopropyl-N-(2-pyridin-2-ylethyl)benzenesulfonamide

In an analogous manner to Example 356, 5-[(3-chloro-4-fluorophenyl)sulfonyl]-2-isopropyl-N-(2-pyridin-2-ylethyl)benzenesulfonamide was used to prepare 5-{[3-chloro-4-(methylamino)phenyl]sulfonyl}-2-isopropyl-N-(2-pyridin-2-ylethyl)benzenesulfonamide.

MS (ES) m/z 506.1.

### EXAMPLE 606

### 2-chloro-5-[(3-methoxyphenyl)sulfonyl]-N-(tetrahydro-2H-pyran-4-yl)benzenesulfonamide

Step 1: Following the same procedure described in Example 474 (Step 3), 3-methoxybenzene sulfonyl chloride was used to prepare 3-methoxybenzenesulfonyl fluoride.

Step 2: Following the same procedure described in Example 474 (Step 4), 5-bromo-2-chloro-N-(tetrahydro-2H-pyran-4-yl)benzenesulfonamide and 3-methoxybenzene sulfonyl fluoride were used to prepare 2-chloro-5-{(3-methoxyphenyl)sulfonyl]-N-(tetrahydro-2H-pyran-4-yl)benzenesulfonamide.

MS (ES) m/z 444.0.

### EXAMPLE 607

### 2-chloro-5-[(1-methyl-1H-indol-5-yl)sulfonyl]-N-(tetrahydro-2H-pyran-4-yl)benzenesulfonamide

In an analogous manner to Example 597,

Step 3: 5-bromo-2-chloro-N-(tetrahydro-2H-pyran-4-yl)benzenesulfonamide was used to prepare 2-chloro-5-[(1-methyl-1H-indol-5-yl)sulfonyl]-N-(tetrahydro-2H-pyran-4-yl)benzenesulfonamide.

MS (ES) m/z 467.0.

### EXAMPLE 608

### 5-(phenylsulfonyl)-N-(tetrahydro-2H-pyran-4-yl)-2-(trifluoromethyl)benzenesulfonamide

A mixture of 5-Benzenesulfonyl-2-trifluoromethyl-benzenesulfinic acid (0.47 g, 1.35 mmol), prepared in the same manner as described in step 5 of Example 570, N-Chlorosuccinimide (0.18 g, 1.36 mmol), and triethylamine (0.38 mL, 2.7 mmol) in 20 mL methylene chloride was stirred under nitrogen for thirty minutes at room temperature. Tetrahydro-2H-pyran-4-amine (0.14 g, 1.35 mmol) was slowly syringed into the flask and the reaction was stirred under nitrogen overnight at room temperature. The solvent was removed under reduced pressure and then the residue was partitioned between methylene chloride and 2 N HCl. The organic layer was separated and the aqueous layer extracted three times with methylene chloride. The combined extracts were dried (anhydrous MgSO₄), filtered and the solvent removed under reduced pressure to produce 0.51 g of a dark brown solid. The solid was taken up in methylene chloride and purified on a Horizon™ Flash Collector (Column: Biotage Si 25+M) using a linear gradient of 5% methylene chloride-hexane to 100% methylene chloride as the eluent. Isolation of the main component gave the title compound 5-(phenylsulfonyl)-N-(tetrahydro-2H-pyran-4-yl)-2-(trifluoromethyl)benzenesulfonamide (55.1 mg, 10%) as a white solid, mp 155-158° C.

MS (ES) m/z 448.0;

Anal. Calcd for C₁₈H₁₈F₃NO₅S₂: C, 48.10; H, 4.04; N, 3.12. Found: C, 48.12; H, 3.81; N, 2.88.

### EXAMPLE 609

### 2,4-Diisopropyl-N-(2-phenylethyl)-5-(phenylsulfonyl)benzenesulfonamide

### Step a) 2,4-Diisopropyl-1-(phenylsulfonyl)benzene

A stirred solution of 1,3-diisopropylbenzene (16.25 g, 100 mmol) and benzenesulfonyl chloride (8.8 g, 50 mmol) was cooled to −20° C. and treated slowly under nitrogen with solid anhydrous aluminum chloride (8.0 g, 60 mmol). After stirring neat for 4 hours at room temperature, the mixture was poured slowly into ice water and extracted with ethyl acetate (2×). The organic phase was washed with a saturated, aqueous sodium chloride solution, dried over anhydrous sodium sulfate, and filtered through a short column of silica gel. The filtrate was evaporated in vacuo to yield a crude oil. The crude oil was purified by preparative liquid chromatography on a Biotage® 40 Mi column of pre-packed silica gel (90 g) (multiple runs), eluting with a gradient of 0%-25% methyl tert-butyl ether in hexane at a flow rate of 50 mL/min, to afford 2,4-diisopropyl-1-(phenylsulfonyl)benzene (5.07 g, 33%), as a clear oil which crystallized on standing to a homogeneous, amorphous solid, m.p. 105-107° C.;

MS (−ESI), m/z: 301 [M−H]⁻;

HRMS: calcd for C₁₈H₂₂O₂S, 302.13405; found (EI, M+.), 302.1332;

HPLC purity 100% at 210-370 nm, 10.5 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### Step b) 2,4-Diisopropyl-5-(phenylsulfonyl)benzenesulfonyl chloride

2,4-Diisopropyl-1-(phenylsulfonyl)benzene (3.01 g, 10 mmol) was heated with stirring at 60° C. for 30 minutes under nitrogen with chlorosulfonic acid (6.7 mL, 11.7 g, 100 mmol). The mixture was cooled to room temperature, poured slowly into a cold solution of 1N hydrochloric acid, and extracted with ethyl acetate (2×.). The organic phase was washed with a saturated, aqueous sodium chloride solution, dried over anhydrous sodium sulfate, filtered, and the solvent concentrated in vacuo to a crude oil. The crude oil was crystallized from diethyl ether-hexane to afford 2,4-diisopropyl-5-(phenylsulfonyl)benzenesulfonyl chloride (2.92 g, 73%) as a colorless, crystalline solid, which was utilized in subsequent reactions.

### Step c) 2,4-Diisopropyl-N-(2-phenylethyl)-5-(phenylsulfonyl)benzenesulfonamide

A stirred solution of 2,4-diisopropyl-5-(phenylsulfonyl)benzenesulfonyl chloride (0.20 g, 0.5 mmol) in dichloromethane (10 mL) was treated dropwise under nitrogen with a solution of phenethylamine (0.12 g, 1.0 mmol) in dichloromethane. After stirring for one hour at room temperature or until the reaction was complete, the mixture was purified by preparative liquid chromatography on a Biotage® 40 Si column of pre-packed silica gel (45 g), eluting with a gradient of 0%-25% methyl tert-butyl ether in hexane at a flow rate of 50 mL/min. After evaporation of the solvent under vacuum, the title compound, 2,4-diisopropyl-N-(2-phenylethyl)-5-(phenylsulfonyl)benzenesulfonamide (0.22 g, 90%), was obtained as a homogeneous, colorless, crystalline solid, m.p. 156-158° C.;

MS (−ESI), m/z: 483.9.1 [M−H]⁻;

HRMS: calcd for C₂₆H₃₁NO₄S₂+H+, 486.17673; found (ESI, [M+H]⁺), 486.1773;

HPLC purity 100% at 210-370 nm, 10.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 610

### 2,4-Diisopropyl-5-(phenylsulfonyl)-N-(tetrahydro-2H-pyran-4-yl)benzenesulfonamide

The title compound was prepared from 2,4-diisopropyl-5-(phenylsulfonyl)benzenesulfonyl chloride (0.20 g, 0.5 mmol) and tetrahydro-pyran-4-ylamine (0.10 g, 1.0 mmol) according to the procedure and in the same manner as described in Example 609, step c. The crude product was purified by preparative liquid chromatography on a Biotage® 40 Si column of pre-packed silica gel (45 g), eluting with a gradient of 30%-70% methyl tert-butyl ether in hexane at a flow rate of 50 mL/min, to afford 2,4-diisopropyl-5-(phenylsulfonyl)-N-(tetrahydro-2H-pyran-4-yl)benzenesulfonamide (0.20 g, 87%), as a homogeneous, colorless, crystalline solid, m.p. 159-160° C.;

MS (⁻ESI), m/z: 464.0 [M−H]⁻;

HRMS: calcd for C₂₃H₃₁NO₅S₂+H+, 466.17164; found (ESI, M+H), 466.1722;

HPLC purity 100% at 210-370 nm, 9.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 611

### 2,4-Diisopropyl-5-(phenylsulfonyl)-N-(tetrahydro-2H-pyran-4-ylmethyl)benzenesulfonamide

The title compound was prepared from 2,4-diisopropyl-5-(phenylsulfonyl)benzenesulfonyl chloride (0.20 g, 0.5 mmol) and (tetrahydro-pyran-4-yl)methylamine (0.12 g, 1.0 mmol) according to the procedure and in the same manner as described in Example 609, step c. The crude product was purified by preparative liquid chromatography on a Biotage® 40 Si column of pre-packed silica gel (45 g), eluting with a gradient of 30%-70% methyl tert-butyl ether in hexane at a flow rate of 50 mL/min, to afford 2,4-diisopropyl-5-(phenylsulfonyl)-N-(tetrahydro-2H-pyran-4-ylmethyl)benzenesulfonamide (0.22 g, 93%), as a homogeneous, colorless, crystalline solid, m.p. 148-150° C.;

MS (+ESI), m/z: 480 [M+H]⁺;

MS (−ESI), m/z: 478 [M−H]⁻;

HPLC purity 100% at 210-370 nm, 9.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 612

### 2,4-Diisopropyl-5-(phenylsulfonyl)-N-[2-(tetrahydro-2H-pyran-4-yl)ethyl]benzenesulfonamide

The title compound was prepared from 2,4-diisopropyl-5-(phenylsulfonyl)benzenesulfonyl chloride (0.20 g, 0.5 mmol) and 2-(tetrahydro-pyran-4-yl)ethylamine (0.12 g, 1.0 mmol) according to the procedure and in the same manner as described in Example 609, step c. The crude product was purified by preparative liquid chromatography on a Biotage® 40 Si column of pre-packed silica gel (45 g), eluting with a gradient of 30%-70% methyl tert-butyl ether in hexane at a flow rate of 50 mL/min, to afford 2,4-diisopropyl-5-(phenylsulfonyl)-N-[2-(tetrahydro-2H-pyran-4-yl)ethyl]benzenesulfonamide (0.23 g, 91%), as a homogeneous, colorless, crystalline solid, m.p. 90-92° C.;

MS (+ESI), m/z: 494 [M+H]⁺;

MS (−ESI), m/z: 492 [M−H]⁻;

HPLC purity 100% at 210-370 nm, 10.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 613

### N-Cyclopentyl-2,4-diisopropyl-5-(phenylsulfonyl)benzenesulfonamide

The title compound was prepared from 2,4-diisopropyl-5-(phenylsulfonyl)benzenesulfonyl chloride (0.20 g, 0.5 mmol) and cyclopentylamine (0.09 g, 1.0 mmol) according to the procedure and in the same manner as described in Example 609, step c. The crude product was purified by preparative liquid chromatography on a Biotage® 40 Si column of pre-packed silica gel (45 g), eluting with a gradient of 0%-25% methyl tert-butyl ether in hexane at a flow rate of 50 mL/min, to afford N-cyclopentyl-2,4-diisopropyl-5-(phenylsulfonyl)benzenesulfonamide (0.17 g, 76%), as a homogeneous, colorless, crystalline solid, m.p. 179-181° C.;

MS (+ESI), m/z: 450 [M+H]⁺;

MS (−ESI), m/z: 448 [M−H]⁻;

HPLC purity 100% at 210-370 nm, 10.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 614

### 2,4-Diisopropyl-5-(phenylsulfonyl)-N-(2-pyridin-2-ylethyl)benzenesulfonamide

The title compound was prepared from 2,4-diisopropyl-5-(phenylsulfonyl)benzenesulfonyl chloride (0.20 g, 0.5 mmol) and 2-(pyridin-2-yl)ethylamine (0.12 g, 1.0 mmol) according to the procedure and in the same manner as described in Example 609, step c. The crude product was purified by preparative liquid chromatography on a Biotage® 40 Si column of pre-packed silica gel (45 g), eluting with a gradient of 50%-100% methyl tert-butyl ether in hexane at a flow rate of 50 mL/min, to afford 2,4-diisopropyl-5-(phenylsulfonyl)-N-(2-pyridin-2-ylethyl)benzenesulfonamide (0.21 g, 84%), as a homogeneous, colorless, crystalline solid, m.p. 189-191° C.;

MS (+ESI), m/z: 487 [M+H]⁺;

MS (−ESI), m/z: 485 [M−H]⁻;

HPLC purity 100% at 210-370 nm, 9.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 615

### N-[3-(1H-Imidazol-1-yl)propyl]-2,4-diisopropyl-5-(phenylsulfonyl)benzenesulfonamide

The title compound was prepared from 2,4-diisopropyl-5-(phenylsulfonyl)benzenesulfonyl chloride (0.20 g, 0.5 mmol) and 3-(1H-imidazol-1-yl)propylamine (0.13 g, 1.0 mmol) according to the procedure and in the same manner as described in Example 609, step c. The crude product was purified by preparative liquid chromatography on a Biotage® 40 Si column of pre-packed silica gel (45 g), eluting with a gradient of 0%-4% methanol in dichloromethane at a flow rate of 50 mL/min, to afford N-[3-(1H-imidazol-1-yl)propyl]-2,4-diisopropyl-5-(phenylsulfonyl)benzenesulfonamide (0.07 g, 27%), as a homogeneous, colorless, crystalline solid, m.p. 203-205° C.;

MS (−ESI), m/z: 488.2 [M−H]⁻;

HPLC purity 100% at 210-370 nm, 8.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 616

### 2,4-Diisopropyl-5-(phenylsulfonyl)-N-(2-pyridin-3-ylethyl)benzenesulfonamide

The title compound was prepared from 2,4-diisopropyl-5-(phenylsulfonyl)benzenesulfonyl chloride (0.20 g, 0.5 mmol) and 2-(pyridin-3-yl)ethylamine (0.12 g, 1.0 mmol) according to the procedure and in the same manner as described in Example 609, step c. The crude product was purified by preparative liquid chromatography on a Biotage® 40 Si column of pre-packed silica gel (45 g), eluting with a gradient of 50%-100% methyl tert-butyl ether in hexane at a flow rate of 50 mL/min, to afford 2,4-diisopropyl-5-(phenylsulfonyl)-N-(2-pyridin-3-ylethyl)benzenesulfonamide (0.15 g, 60%), as a homogeneous, colorless, crystalline solid, m.p. 163-165° C.;

MS (−ESI), m/z: 485.2 [M−H]⁻;

HPLC purity 100% at 210-370 nm, 9.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 617

### 2,4-Diisopropyl-5-(phenylsulfonyl)-N-(2-pyridin-4-ylethyl)benzenesulfonamide

The title compound was prepared from 2,4-diisopropyl-5-(phenylsulfonyl)benzenesulfonyl chloride (0.20 g, 0.5 mmol) and 2-(pyridin-4-yl)ethylamine (0.12 g, 1.0 mmol) according to the procedure and in the same manner as described in Example 609, step c. The crude product was purified by preparative liquid chromatography on a Biotage® 40 Si column of pre-packed silica gel (45 g), eluting with a gradient of 50%-100% methyl tert-butyl ether in hexane at a flow rate of 50 mL/min, to afford 2,4-diisoipropyl-5-(phenylsulfonyl)-N-(2-pyridin-4-ylethyl)benzenesulfonamide (0.12 g, 49%), as a homogeneous, colorless, crystalline solid, m.p. 178-180° C.;

MS (−ESI), m/z: 485.1 [M−H]⁻;

HPLC purity 100% at 210-370 nm, 9.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 618

### N-(2,3-Dihydro-1H-inden-2-yl)-2,4-diisopropyl-5-(phenylsulfonyl)benzenesulfonamide

The title compound was prepared from 2,4-diisopropyl-5-(phenylsulfonyl)benzenesulfonyl chloride (0.20 g, 0.5 mmol) and indan-2-ylamine (0.13 g, 1.0 mmol) according to the procedure and in the same manner as described in Example 609, step c. The crude product was purified by preparative liquid chromatography on a Biotage® 40 Si column of pre-packed silica gel (45 g), eluting with a gradient of 0%-25% methyl tert-butyl ether in hexane at a flow rate of 50 mL/min, to afford N-(2,3-dihydro-1H-inden-2-yl)-2,4-diisopropyl-5-(phenylsulfonyl)benzenesulfonamide (0.22 g, 90%), as a homogeneous, colorless, crystalline solid, m.p. 215-217° C.;

MS (−ESI), m/z: 496.1 [M−H]⁻;

HPLC purity 100% at 210-370 nm, 11.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 619

### 5-[(4-Fluorophenyl)sulfonyl]-2,4-diisopropyl-N-(2-phenylethyl)benzenesulfonamide

### Step a) 1-[(4-Fluorophenyl)sulfonyl]-2,4-diisopropylbenzene

A stirred solution of 1,3-diisopropylbenzene (16.25 g, 100 mmol) and 4-fluorobenzenesulfonyl chloride (9.73 g, 50 mmol) was cooled to −20° C. and treated slowly under nitrogen with solid anhydrous aluminum chloride (8.0 g, 60 mmol). After stirring neat for 4 hours at room temperature, the mixture was poured slowly into ice water and extracted with ethyl acetate (2×). The organic phase was washed with a saturated, aqueous sodium chloride solution, dried over anhydrous sodium sulfate, and filtered through a short column of silica gel. The filtrate was evaporated in vacuo to yield a crude oil. The crude oil was purified by preparative liquid chromatography on a Biotage® 40 Mi column of pre-packed silica gel (90 g) (multiple runs), eluting with a gradient of 0%-25% methyl tert-butyl ether in hexane at a flow rate of 50 mL/min, to afford 1-[(4-fluorophenyl)sulfonyl]-2,4-diisopropylbenzene (9.71 g, 60%), as a clear oil which crystallized on standing to a homogeneous, amorphous solid, m.p. 75-80° C.;

MS (EI), m/z: 320 [M⁻]⁺;

HRMS: calcd for C₁₈H₂₁FO₂S, 320.12463; found (ESI, M+.), 320.1237;

HPLC purity 100% at 210-370 nm, 10.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### Step b) 5-[(4-Fluorophenyl)sulfonyl]-2,4-diisopropylbenzenesulfonyl chloride

1-[(4-Fluorophenyl)sulfonyl]-2,4-diisopropylbenzene (3.20 g, 10 mmol) was heated with stirring at 60° C. for 30 minutes under nitrogen with chlorosulfonic acid (6.7 mL, 11.7 g, 100 mmol). The mixture was cooled to room temperature, poured slowly into a cold solution of 1N hydrochloric acid, and extracted with ethyl acetate (2×). The organic phase was washed with a saturated, aqueous sodium chloride solution, dried over anhydrous sodium sulfate, filtered, and the solvent concentrated in vacuo to a crude oil. The crude oil was crystallized from diethyl ether-hexane to afford 5-[(4-fluorophenyl)sulfonyl]-2,4-diisopropylbenzenesulfonyl chloride (3.11 g, 74%) as a colorless, crystalline solid, which was utilized in subsequent reactions.

### Step c) 5-[(4-Fluorophenyl)sulfonyl]-2,4-diisopropyl-N-(2-phenylethyl)benzenesulfonamide

A stirred solution of 5-[(4-fluorophenyl)sulfonyl]-2,4-diisopropylbenzenesulfonyl chloride (0.21 g, 0.5 mmol) in dichloromethane (10 mL) was treated dropwise under nitrogen with a solution of phenethylamine (0.12 g, 1.0 mmol) in dichloromethane. After stirring for one hour at room temperature or until the reaction was complete, the mixture was purified by preparative liquid chromatography on a Biotage® 40 Si column of pre-packed silica gel (45 g), eluting with a gradient of 0%-25% methyl tert-butyl ether in hexane at a flow rate of 50 mL/min. After evaporation of the solvent under vacuum, the title compound, 5-[(4-fluorophenyl)sulfonyl]-2,4-diisopropyl-N-(2-phenylethyl)benzenesulfonamide (0.24 g, 95%), was obtained as a homogeneous, colorless, crystalline solid, m.p. 154-156° C.;

MS (−ESI), m/z: 502 [M−H]⁻;

HPLC purity 100% at 210-370 nm, 10.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 620

### 5-[(4-Fluorophenyl)sulfonyl]-2,4-diisopropyl-N-(tetrahydro-2H-pyran-4-yl)benzenesulfonamide

The title compound was prepared from 5-[(4-fluorophenyl)sulfonyl]-2,4-diisopropylbenzenesulfonyl chloride (0.32 g, 0.75 mmol) and tetrahydro-pyran-4-ylamine (0.15 g, 1.5 mmol) according to the procedure and in the same manner as described in Example 619, step c. The crude product was purified by preparative liquid chromatography on a Biotage® 40 Si column of pre-packed silica gel (45 g), eluting with a gradient of 30%-70% methyl tert-butyl ether in hexane at a flow rate of 50 mL/min, to afford 5-[(4-fluorophenyl)sulfonyl]-2,4-diisopropyl-N-(tetrahydro-2H-pyran-4-yl)benzenesulfonamide (0.31 g, 86%), as a homogeneous, colorless, crystalline solid, m.p. 183-186° C.;

MS (−ESI), m/z: 481.8 [M−H]⁻;

HPLC purity 100% at 210-370 nm, 10.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 621

### N-Cyclopentyl-5-[(4-fluorophenyl)sulfonyl]-2,4-diisopropylbenzenesulfonamide

The title compound was prepared from 5-[(4-fluorophenyl)sulfonyl]-2,4-diisopropylbenzenesulfonyl chloride (0.21 g, 0.5 mmol) and cyclopentylamine (0.09 g, 1.0 mmol) according to the procedure and in the same manner as described in Example 619, step c. The crude product was purified by preparative liquid chromatography on a Biotage® 40 Si column of pre-packed silica gel (45 g), eluting with a gradient of 0%-25% methyl tert-butyl ether in hexane at a flow rate of 50 mL/min, to afford N-cyclopentyl-5-[(4-fluorophenyl)sulfonyl]-2,4-diisopropylbenzenesulfonamide (0.20 g, 85%), as a homogeneous, colorless, crystalline solid, m.p. 202-205° C.;

MS (−ESI), m/z: 465.7 [M−H]⁻;

HPLC purity 100% at 210-370 nm, 10.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 622

### 2-chloro-5-[(4-fluorophenyl)sulfonyl]-N-(tetrahydro-2H-pyran-4-yl)benzenesulfonamide

Step 1: Following the same procedure described in Example 474 (Step 3), 4-fluorobenzene sulfonyl chloride was used to prepare 4-fluorobenzenesulfonyl fluoride.

Step 2: Following the same procedure described in Example 474 (Step 4), 5-bromo-2-chloro-N-(tetrahydro-2H-pyran-4-yl)benzenesulfonamide and 4-fluorobenzene sulfonyl fluoride were used to prepare 2-chloro-5-[(4-fluorophenyl)sulfonyl]-N-(tetrahydro-2H-pyran-4-yl)benzenesulfonamide.

MS (ES) m/z 432.0.

### EXAMPLE 623

### tert-butyl 4-({[2-chloro-5-(phenylsulfonyl)phenyl]sulfonyl}amino)piperidine-1-carboxylate

In an analogous manner to Example 334,

Step 3: 4-aminopiperidine-1-carboxylic acid tert-butyl ester was used to prepare tert-butyl 4-({[2-chloro-5-(phenylsulfonyl)phenyl]sulfonyl}amino)piperidine-1-carboxylate.

MS (ES) m/z 513.1.

### EXAMPLE 624

### 2-chloro-5-(phenylsulfonyl)-N-piperidin-4-ylbenzenesulfonamide

tert-butyl 4-({[2-chloro-5-(phenylsulfonyl)phenyl]sulfonyl}amino)piperidine-1-carboxylate (0.65 g, 1.26 mmol) was stirred in HCl 4 M in dioxane (5 mL) for 1 hr. The mixture was neutralized with a saturated aqueous sodium bicarbonate solution and extracted with ethyl acetate. The organic layer was dried over magnesium sulfate, filtered, and concentrated to give 2-chloro-5-(phenylsulfonyl)-N-piperidin-4-ylbenzenesulfonamide (0.37 g, 70%).

MS (ES) m/z 412.9.

### EXAMPLE 625

### tert-butyl 4-[4-({4-isopropyl-3-[(tetrahydro-2H-pyran-4-ylamino)sulfonyl]phenyl}sulfonyl)phenyl]piperazine-1-carboxylate

In an analogous manner to Example 547, 5-[(4-fluorophenyl)sulfonyl]-2-isopropyl-N-tetrahydro-2H-pyran-4-ylbenzenesulfonamide, piperazine-1-carboxylic acid tert-butyl ester, and dimethylacetamide as solvent were used to prepare tert-butyl 4-[4-({4-isopropyl-3-[(tetrahydro-2H-pyran-4-ylamino)sulfonyl]phenyl}sulfonyl)phenyl]piperazine-1-carboxylate.

MS (ESI +) m/z 608;

HPLC purity 97.2% at 210-370 nm, 10.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 626

### 5-({4-cis-3,5-dimethylpiperazin-1-ylphenyl}sulfonyl)-2-isopropyl-N-(tetrahydro-2H-pyran-4-yl)benzenesulfonamide

In an analogous manner to Example 547, 5-[(4-fluorophenyl)sulfonyl]-2-isopropyl-N-tetrahydro-2H-pyran-4-ylbenzenesulfonamide, cis-2,6-dimethylpiperazine, and dimethylacetamide as solvent were used to prepare 5-({4-cis-3,5-dimethylpiperazin-1-ylphenyl}sulfonyl)-2-isopropyl-N-(tetrahydro-2H-pyran-4-yl)benzenesulfonamide.

MS (ESI+) m/z 536;

HPLC purity 100% at 210-370 nm, 7.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 627

### 5-({4-trans-2,5-dimethylpiperazin-1-ylphenyl}sulfonyl)-2-isopropyl-N-(tetrahydro-2H-pyran-4-yl)benzenesulfonamide

In an analogous manner to Example 547, 5-[(4-fluorophenyl)sulfonyl]-2-isopropyl-N-tetrahydro-2H-pyran-4-ylbenzenesulfonamide, cis-2,6-dimethylpiperazine, and dimethylacetamide as solvent were used to prepare 5-({4-trans-2,5-dimethylpiperazin-1-ylphenyl}sulfonyl)-2-isopropyl-N-(tetrahydro-2H-pyran-4-yl)benzenesulfonamide.

MS (ESI+) m/z 536;

HPLC purity 100% at 210-370 nm, 7.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 628

### 2-isopropyl-5-[(4-piperazin-1-ylphenyl)sulfonyl]-N-(tetrahydro-2H-pyran-4-yl)benzenesulfonamide

To a stirred slurry of tert-butyl 4-[4-({4-isopropyl-3-[(tetrahydro-2H-pyran-4-ylamino)sulfonyl]phenyl}sulfonyl)phenyl]piperazine-1-carboxylate (50 mg, 0.08 mmol) in anhydrous tetrahydrofuran (1 mL) was added a 2.0 M HCl solution in tetrahydrofuran (0.5 mL). After 16 hours the starting material was still present and an additional 0.5 mL of the HCl solution was added and the slurry was stirred at room temperature for an additional 12 h where upon a predominately single product was observed. An aqueous solution of 2 M HCl was added and the aqueous phase was extracted with ethyl acetate (2×1 mL). The aqueous phase was then neutralized with a saturated aqueous solution of sodium bicarbonate until it was basic as determined by pH strip. The aqueous phase was then extracted with ethyl acetate (3×1 mL) and the combined organic phases were dried (MgSO₄), filtered and concentrated to afford 2-isopropyl-5-[(4-piperazin-1-ylphenyl)sulfonyl]-N-(tetrahydro-2H-pyran-4-yl)benzenesulfonamide (31 mg, 75%).

MS (ES−) m/z 506.1;

HPLC purity 100% at 210-370 nn, 6.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 629

### 5-[(4-Fluorophenyl)sulfonyl]-2,4-diisopropyl-N-(tetrahydro-2H-pyran-4-ylmethyl)benzenesulfonamide

The title compound was prepared from 5-[(4-fluorophenyl)sulfonyl]-2,4-diisopropylbenzenesulfonyl chloride (0.21 g, 0.5 mmol) and (tetrahydro-pyran-4-yl)methylamine (0.12 g, 1.0 mmol) according to the procedure and in the same manner as described in Example 619, step c. The crude product was purified by preparative liquid chromatography on a Biotage® 40 Si column of pre-packed silica gel (45 g), eluting with a gradient of 30%-70% methyl tert-butyl ether in hexane at a flow rate of 50 mL/min, to afford 5-8 (4-fluorophenyl)sulfonyl]-2,4-diisopropyl-N-(tetrahydro-2H-pyran-4-ylmethyl)benzenesulfonamide (0.22 g, 87%), as a homogeneous, colorless, crystalline solid, m.p. 200-202° C.;

MS (−ESI), m/z: 496.1 [M−H]⁻;

HPLC purity 100% at 210-370 nm, 10.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 630

### 5-[(4-Fluorophenyl)sulfonyl]-2,4-diisopropyl-N-[2-(tetrahydro-2H-pyran-4-yl)ethyl]benzenesulfonamide

The title compound was prepared from 5-[(4-fluorophenyl)sulfonyl]-2,4-diisopropylbenzenesulfonyl chloride (0.21 g, 0.5 mmol) and 2-(tetrahydro-pyran-4-yl)ethylamine (0.13 g, 1.0 mmol) according to the procedure and in the same manner as described in Example 619, step c. The crude product was purified by preparative liquid chromatography on a Biotage® 40 Si column of pre-packed silica gel (45 g), eluting with a gradient of 30%-70% methyl tert-butyl ether in hexane at a flow rate of 50 mL/min, to afford 5-[(4-fluorophenyl)sulfonyl]-2,4-diisopropyl-N-[2-(tetrahydro-2H-pyran-4-yl)ethyl]benzenesulfonamide (0.18 g, 69%), as a homogeneous, colorless, crystalline solid, m.p: 141-143° C.;

MS (−ESI), m/z: 510.2 [M−H]⁻;

HPLC purity 100% at 210-370 nr, 10.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 631

### 5-[(4-Fluorophenyl)sulfonyl]-2,4-diisopropyl-N-(2-pyridin-2-ylethyl)benzenesulfonamide

The title compound was prepared from 5-[(4-fluorophenyl)sulfonyl]-2,4-diisopropylbenzenesulfonyl chloride (0.21 g, 0.5 mmol) and 2-(pyridin-2-yl)ethylamine (0.12 g, 1.0 mmol) according to the procedure and in the same manner as described in Example 619, step c. The crude product was purified by preparative liquid chromatography on a Biotage® 40 Si column of pre-packed silica gel (45 g), eluting with a gradient of 50%-100% methyl tert-butyl ether in hexane at a flow rate of 50 mL/min, to afford 5-[(4-fluorophenyl)sulfonyl]-2,4-diisopropyl-N-(2-pyridin-2-ylethyl)benzenesulfonamide (0.22 g, 86%), as a homogeneous, colorless, crystalline solid, m.p. 159-161° C.;

MS (−ESI), m/z: 503.1 [M−H]⁻;

HPLC purity 100% at 210-370 nm, 10.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 632

### 5-[(4-Fluorophenyl)sulfonyl]-2,4-diisopropyl-N-(2-pyridin-3-ylethyl)benzenesulfonamide

The title compound was prepared from 5-[(4-fluorophenyl)sulfonyl]-2,4-diisopropylbenzenesulfonyl chloride (0.21 g, 0.5 mmol) and 2-(pyridin-3-yl)ethylamine (0.12 g, 1.0 mmol) according to the procedure and in the same manner described in Example 619, step c. The crude product was purified by preparative liquid chromatography on a Biotage® 40 Si column of pre-packed silica gel (45 g), eluting with a gradient of 50%-100% methyl tert-butyl ether in hexane at a flow rate of 50 mL/min, to afford 5-[(4-fluorolphenyl)sulfonyl]-2,4-diisopropyl-N-(2-pyridin-3-ylethyl)benzenesulfonamide (0.18 g, 71%), as a homogeneous, colorless, crystalline solid, m.p. 148-150° C.;

MS (−ESI), m/z: 503.1 [M−H]⁻;

HPLC purity 100% at 210-370 nm, 9.8 min.; Xteffa RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 633

### 5-[(4-Fluorophenyl)sulfonyl]-2,4-diisopropyl-N-(2-pyridin-4-ylethyl)benzenesulfonamide

The title compound was prepared from 5-[(4-fluorophenyl)sulfonyl]-2,4-diisopropylbenzenesulfonyl chloride (0.21 g, 0.5 mmol) and 2-(pyridin-4-yl)ethylamine (0.12 g, 1.0 mmol) according to the procedure and in the same manner described in Example 619, step c. The crude product was purified by preparative liquid chromatography on a Biotage® 40 Si column of pre-packed silica gel (45 g), eluting with a gradient of 50%-100% methyl tert-butyl ether in hexane at a flow rate of 50 mL/min, to afford 5-[(4-fluorophenyl)sulfonyl]-2,4-diisopropyl-N-(2-pyridin-4-ylethyl)benzenesulfonamide (0.14 g, 55%), as a homogeneous, colorless, crystalline solid, m.p. 210-211° C.;

MS (−ESI), m/z: 503.1 [M−H]⁻;

HPLC purity 100% at 210-370 mn, 9.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 634

### N-(2,3-Dihydro-1H-inden-2-yl)-5-[(4-fluorophenyl)sulfonyl]-2,4-diisopropylbenzenesulfonamide

The title compound was prepared from 5-[(4-fluorophenyl)sulfonyl]-2,4-diisopropylbenzenesulfonyl chloride (0.21 g, 0.5 mmol) and indan-2-ylamine (0.13 g, 1.0 mmol) according to the procedure and in the same manner as described in Example 619, step c. The crude product was purified by preparative liquid chromatography on a Biotage® 40 Si column of pre-packed silica gel (45 g), eluting with a gradient of 0%-25% methyl tert-butyl ether in hexane at a flow rate of 50 mL/min, to afford N-(2,3-dihydro-1H-inden-2-yl)-5-[(4-fluorophenyl)sulfonyl]-2,4-diisopropylbenzenesulfonamide (0.23 g, 89%), as a homogeneous, colorless, crystalline solid, m.p. 202-204° C.;

MS (−ESI), m/z: 514.1 [M−H]⁻;

HPLC purity 100% at 210-370 nm, 11.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 635

### 1-{[2-chloro-5-(phenylsulfonyl)phenyl]sulfonyl}-4-pyrrolidin-1-ylpiperidine

In an analogous manner to Example 334,

Step 3: 4-pyrrolidin-1-yl-piperidine was used to prepare 1-{[2-chloro-5-(phenylsulfonyl)phenyl]sulfonyl}-4-pyrrolidin-1-ylpiperidine.

MS (ESI) m/z 469.

### EXAMPLE 636

### 4-[2-(4-{[2-chloro-5-(phenylsulfonyl)phenyl]sulfonyl}piperazin-1-yl)ethyl]morpholine

In an analogous manner to Example 334,

Step 3: 4-(2-piperazin-1-ylethyl)-morpholine was used to prepare 4-[2-(4-{[2-chloro-5-(phenylsulfonyl)phenyl]sulfonyl}piperazin-1-yl)ethyl]morpholine.

MS (ESI) m/z 514.

### EXAMPLE 637

### 1-(1,3-benzodioxol-5-ylmethyl)-4-{[2-chloro-5-(phenylsulfonyl)phenyl]sulfonyl}piperazine

In an analogous manner to Example 334,

Step 3: 1-benzo[1,3]dioxol-5-ylmethyl-piperazine was used to prepare 1-(1,3-benzodioxol-5-ylmethyl)-4-{[2-chloro-5-(phenylsulfonyl)phenyl]sulfonyl}piperazine.

MS (ESI) m/z 535.

### EXAMPLE 638

### tert-butyl(1-{[2-chloro-5-(phenylsulfonyl)phenyl]sulfonyl}pyrrolidin-3-yl)carbamate

In an analogous manner to Example 334,

Step 3: pyrrolidin-3-yl-carbamic acid tert-butyl ester,was used to prepare tert-butyl(1-{[2-chloro-5-(phenylsulfonyl)phenyl]sulfonyl}pyrrolidin-3-yl)carbamate.

MS (ESI) m/z 501.

### EXAMPLE 639

### tert-butyl(1-{[2-chloro-5-(phenylsulfonyl)phenyl]sulfonyl}piperidin-4-yl)carbamate

In an analogous manner to Example 334,

Step 3: piperidin-4-yl-carbamic acid tert-butyl ester was used to prepare tert-butyl(1-{[2-chloro-5-(phenylsulfonyl)phenyl]sulfonyl}piperidin-4-yl)carbamate.

MS (ESI) m/z 515.

### EXAMPLE 640

### 2-chloro-5-(phenylsulfonyl)-N-(2-pyridin-3-ylethyl)benzenesulfonamide

In an analogous manner to Example 334,

Step 3: 2-pyridin-3-yl-ethylamine was used to prepare 2-chloro-5-(phenylsulfonyl)-N-(2-pyridin-3-ylethyl)benzenesulfonamide.

MS (ESI) m/z 437.

### EXAMPLE 641

### 2-chloro-5-(phenylsulfonyl)-N-(2-pyridin-4-ylethyl)benzenesulfonamide

In an analogous manner to Example 334,

Step 3: 2-pyridin-4-yl-ethylamine was used to prepare 2-chloro-5-(phenylsulfonyl)-N-(2-pyridin-4-ylethyl)benzenesulfonamide.

MS (ESI) m/z 437.

### EXAMPLE 642

### 2-chloro-N-[3-(4-methylpiperazin-1-yl)propyl]-5-(phenylsulfonyl)benzenesulfonamide

In an analogous manner to Example 334,

Step 3: 3-(4-methylpiperazin-1-yl)-propylamine was used to prepare 2-chloro-N-[3-(4-methylpiperazin-1-yl)propyl]-5-(phenylsulfonyl)benzenesulfonamide.

MS (ESI) m/z 472.

### EXAMPLE 643

### 2-chloro-N-(2-cyanoethyl)-5-(phenylsulfonyl)benzenesulfonamide

In an analogous manner to Example 334,

Step 3: 3-aminopropionitrile was used to prepare 2-chloro-N-(2-cyanoethyl)-5-(phenylsulfonyl)benzenesulfonamide.

MS (ESI) m/z 385.

### EXAMPLE 644

### 1-{[2-chloro-5-(phenylsulfonyl)phenyl]sulfonyl}-N,N-diethylpyrrolidin-3-amine

In an analogous manner to Example 334,

Step 3: diethylpyrrolidin-3-yl-amine was used to prepare 1-{[2-chloro-5-(phenylsulfonyl)phenyl]sulfonyl}-N,N-diethylpyrrolidin-3-amine.

MS (ESI) m/z 457.

### EXAMPLE 645

### ethyl 1-{[2-chloro-5-(phenylsulfonyl)phenyl]sulfonyl}piperidine-3-carboxylate

In an analogous manner to Example 334,

Step 3: piperidine-3-carboxylic acid ethyl ester was used to prepare ethyl 1-{[2-chloro-5-(phenylsulfonyl)phenyl]sulfonyl}piperidine-3-carboxylate.

MS (ESI) m/z 472.

### EXAMPLE 646

### 2-chloro-N-methyl-5-(phenylsulfonyl)-N-(2-pyridin-2-ylethyl)benzenesulfonamide

In an analogous manner to Example 334,

Step 3: methyl-(2-pyridin-2-yl-ethyl)-amine was used to prepare 2-chloro-N-methyl-5-(phenylsulfonyl)-N-(2-pyridin-2-ylethyl)benzenesulfonamide.

MS (ESI) m/z 451.

### EXAMPLE 647

### 2-chloro-5-(phenylsulfonyl)-N-[1-(trifluoroacetyl)piperidin-4-yl]benzenesulfonamide

To a stirred solution of 2-chloro-5-(phenylsulfonyl)-N-piperidin-4-ylbenzenesulfonamide (0.09 g, 0.22 mmol) in dichloromethane (2 mL) was added triethylamine (0.1 mL, 0.72 mmol) and trifluoroacetic anhydride (0.09 g, 0.42 mmol). The resulting solution was stirred for 1 hr and concentrated. The crude mixture was flash column separated using 10-50% ethyl acetate/hexane to give 2-chloro-5-(phenylsulfonyl)-N-[1-(trifluoroacetyl)piperidin-4-yl]benzenesulfonamide (0.04 g, 36%).

MS (ESI) m/z 511.

### EXAMPLE 648

### 2-chloro-N-[1-(2,2-dimethylpropanoyl)piperidin-4-yl]-5-(phenylsulfonyl)benzenesulfonamide

In an analogous manner to Example 647, trimethylacetyl chloride was used to prepare 2-chloro-N-[1-(2,2-dimethylpropanoyl)piperidin-4-yl]-5-(phenylsulfonyl)benzenesulfonamide.

MS (ESI) m/z 499.

### EXAMPLE 649

### N-(tert-butyl)-4-({[2-chloro-5-(phenylsulfonyl)phenyl]sulfonyl}amino)piperidine-1-carboxamide

In an analogous manner to Example 647, tert-butyl isocyanate was used to prepare N-(tert-butyl)-4-({[2-chloro-5-(phenylsulfonyl)phenyl]sulfonyl}amino)piperidine-1-carboxamide.

MS (ESI) m/z 512.

### EXAMPLE 650

### 2-chloro-N-[1-(morpholin-4-ylcarbonyl)piperidin-4-yl]-5-(phenylsulfonyl)benzenesulfonamide

In an analogous manner to Example 647, morpholine-4-carbonyl chloride was used to prepare 2-chloro-N-[1-(morpholin-4-ylcarbonyl)piperidin-4-yl]-5-(phenylsulfonyl)benzenesulfonamide.

MS (ESI) m/z 528.

### EXAMPLE 651

### 2-chloro-N-(1-cyanopiperidin-4-yl)-5-(phenylsulfonyl)benzenesulfonamide

In an analogous manner to Example 647, cyanogens bromide was used to prepare 2-chloro-N-(1-cyanopiperidin-4-yl)-5-(phenylsulfonyl)benzenesulfonamide.

MS (ESI) m/z 440.

### EXAMPLE 652

### 5-[(4-fluorophenyl)sulfonyl]-2-isopropyl-N-[2-(1-oxidopyridin-3-yl)ethyl]benzenesulfonamide

100 mg of 5-[(4-fluorophenyl)sulfonyl]-2-isopropyl-N-(2-pyridin-3-ylethyl)benzenesulfonamide was added to 4 mL of 50% hydrogen peroxide/acetic acid and the reaction was heated at 100° C. for four hours. The reaction was poured onto saturated bicarbonate solution and extracted with dichloromethane. The organic layer was washed with sodium dithionite solution. The organic layer was dried with magnesium sulfate and concentrated. Crude material was purified by flash chromatography using 10% methanol/dichloromethane to give 5-[(4-fluorophenyl)sulfonyl]-2-isopropyl-N-[2-(1-oxidopyridin-3-yl)ethyl]benzenesulfonamide.

MS (ES+) m/z 479

MS (ES−) m/z 477.

### EXAMPLE 653

### 1-{[2-chloro-5-(phenylsulfonyl)phenyl]sulfonyl}-4-[(2,5-dimethyl-1H-pyrrol-1-yl)methyl]piperidine

In an analogous manner to Example 647, 4-(2,5-dimethylpyrrol-1-ylmethyl)-piperidine was used to prepare 1-{[2-chloro-5-(phenylsulfonyl)phenyl]sulfonyl}-4-[(2,5-dimethyl-1H-pyrrol-1-yl)methyl]piperidine.

MS (ES) m/z 507.0.

### EXAMPLE 654

### methyl N-{[2-chloro-5-(phenylsulfonyl)phenyl]sulfonyl}-2-methylalaninate

To a stirred solution of methyl alpha-aminoisobutyrate hydrochloride (1.3 g, 8.53 mmol) in a saturated aqueous sodium bicarbonate solution was added 5-benzenesulfonyl-2-chlorobenzenesulfonyl chloride (1.5 g, 4.27 mmol) dissolved in acetonitrile (10 mL). The resulting solution was stirred vigorously 1 hr and extracted several times with ethyl acetate. The combined organic layers were washed with ammonium chloride solution and concentrated. Flash column separation using 10-50% ethyl acetate/hexane gave methyl N-{[2-chloro-5-(phenylsulfonyl)phenyl]sulfonyl}-2-methylalaninate (0.96 g, 52%)

MS (ESI) m/z 432.

### EXAMPLE 655

### 2-chloro-N-(2-hydroxy-1,1-dimethylethyl)-5-(phenylsulfonyl)benzenesulfonamide

To a stirred solution of methyl N-{[2-chloro-5-(phenylsulfonyl)phenyl]sulfonyl}-2-methylalaninate (1.20 g, 2.78 mmol) in THF (16 mL) at −78° C. was added diisobutylaluminum hydride 1.0 M in toluene (16.0 mL, 16.0 mmol) dropwise over 10 minutes. The resulting solution was stirred for 6 hours at −78° C., quenched with 0.5 M HCl solution and extracted several times with ethyl acetate. The combined organic layer was dried over magnesium sulfate, and concentrated to give 2-chloro-N-(2-hydroxy-1,1-dimethylethyl)-5-(phenylsulfonyl)benzenesulfonamide (0.64 g, 57%).

MS (ES−) m/z 402.0;

HPLC purity 100% at 210-370 nm, 8.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₆H₁₈ClNO₅S₂+H+, 404.03877; found (ESI, [M+H]⁺), 404.0384.

### EXAMPLE 656

### 2-chloro-N-[1-(4-fluorobenzoyl)piperidin-4-yl]-5-(phenylsulfonyl)benzenesulfonamide

In an analogous manner to example 462, 2-chloro-5-(phenylsulfonyl)-N-piperidin-4-ylbenzenesulfonamide and 4-fluorobenzoyl chloride were used to prepare 2-chloro-N-[1-(4-fluorobenzoyl)piperidin-4-yl]-5-(phenylsulfonyl)benzenesulfonamide.

MS (ES−) m/z 534.9;

HPLC purity 100% at 210-370 nm, 9.2 min.; Xterra RP18, 3.5 u, 150×4.6 mm column, 1.2 mL/min, 85/15-5/95 (Ammon. Form. Buff. Ph=3.5/ACN+MeOH) for 1 min, hold 4 min.

HRMS: calcd for C₂₄H₂₂ClFN₂O₅S₂+H+, 537.07154; found (ESI, [M+H]⁺), 537.0699.

### EXAMPLE 657

### 2-chloro-N-[1-(4-cyanobenzoyl)piperidin-4-yl]-5-(phenylsulfonyl)benzenesulfonamide

In an analogous manner to example 462, 2-chloro-5-(phenylsulfonyl)-N-piperidin-4-ylbenzenesulfonamide and 4-cyanobenzoyl chloride were used to prepare 2-chloro-N-[1-(4-cyanobenzoyl)piperidin-4-yl]-5-(phenylsulfonyl)benzenesulfonamide.

MS (ES−) m/z 541.9;

HPLC purity 100% at 210-370 nm, 8.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₅H₂₂ClN₃O₅S₂+H+, 544.07622; found (ESI, [M+H]⁺), 544.075.

### EXAMPLE 658

### 2-chloro-5-(phenylsulfonyl)-N-{1-[4-(trifluoromethyl)benzoyl]piperidin-4-yl}benzenesulfonamide

In an analogous manner to example 462, 2-chloro-5-(phenylsulfonyl)-N-piperidin-4-ylbenzenesulfonamide and 4-trifluoromethylbenzoyl chloride were used to prepare 2-chloro-5-(phenylsulfonyl)-N-{1-[4-(trifluoromethyl)benzoyl]piperidin-4-yl}benzenesulfonamide.

MS (ES−) m/z 584.9;

HPLC purity 94.5% at 210-370 nm, 9.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 660

### 5-(phenylsulfonyl)-N-(2-pyridin-3-ylethyl)-2-(trifluoromethoxy)benzenesulfonamide

Step 1: 1-bromo-4-trifluoromethoxybenzene (0.50 g, 2.07 mmol) was added to chlorosulfonic acid (2 mL, 29.0 mmol) at room temperature. The resulting mixture was stirred 2 hours, poured over ice, and extracted several times with ethyl acetate. The combined organic layer was washed with water, dried over magnesium sulfate and concentrated to give 5-bromo-2-trifluoromethoxybenzene sulfonyl chloride (0.45 g, 64%)

Step 2: In an analogous manner to example 435, 5-bromo-2-trifluoromethoxybenzene sulfonyl chloride and 3-(2-aminoethyl)pyridine were used to prepare 5-bromo-N-(2-pyridin-3-ylethyl)-2-(trifluoromethoxy)benzenesulfonamide.

Step 3: Following the same procedure described on example 474 (Step 4), 5-bromo-N-(2-pyridin-3-ylethyl)-2-(trifluoromethoxy)benzenesulfonamide and benzene sulfonyl fluoride were used to prepare 5-(phenylsulfonyl)-N-(2-pyridin-3-ylethyl)-2-(trifluoromethoxy)benzenesulfonamide.

MS (ES−) m/z 485.0;

HPLC purity 92.5% at 210-370 nm, 8.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₀H₁₇F₃N₂O₅S₂+H+, 487.06037; found (ESI, [M+H]⁺), 487.0615.

### EXAMPLE 662

### 5-(phenylsulfonyl)-N-(tetrahydro-2H-pyran-4-yl)-2-(trifluoromethoxy)benzenesulfonamide

In an analogous manner to Example 660,

Step 2: 5-bromo-2-trifluoromethoxybenzene sulfonyl chloride and tetrahydro-pyran-4-ylamine was used to prepare 5-bromo-N-(tetrahydro-2H-pyran-4-yl)-2-(trifluoromethoxy)benzenesulfonamide

Step 3: 5-bromo-N-(tetrahydro-2H-pyran-4-yl)-2-(trifluoromethoxy)benzenesulfonamide and benzene sulfonyl fluoride were used to prepare 5-(phenylsulfonyl)-N-(tetrahydro-2H-pyran-4-yl)-2-(trifluoromethoxy)benzenesulfonamide.

MS (ES−) m/z 464.0;

HPLC purity 95.2% at 210-370 nm, 8.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₈H₁₈F₃NO₆S₂+H+, 466.06004; found (ESI, [M+H]⁺), 466.0606.

### EXAMPLE 663

### 2-chloro-N-(cyanomethyl)-5-(phenylsulfonyl)benzenesulfonamide

In an analogous manner to example 654, 2-chloro-5-(phenylsulfonyl)-benzenesulfonyl chloride and aminoacetonitrile HCl were used to prepare 2-chloro-N-(cyanomethyl)-5-(phenylsulfonyl)benzenesulfonamide.

MS (ES−) m/z 369.0;

HPLC purity 88.5% at 210-370 nm, 7.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 664

### N-(2-cyanoethyl)-5-[(4-fluorophenyl)sulfonyl]-2-isopropylbenzenesulfonamide

In an analogous manner to example 435, 5-(4-fluoro-benzenesulfonyl)-2-isopropyl-benzenesulfonyl chloride and aminopropionitrile were used to prepare N-(2-cyanoethyl)-5-[(4-fluorophenyl)sulfonyl]-2-isopropylbenzenesulfonamide.

MS (ES−) m/z 409.1;

HPLC purity 99.0% at 210-370 nm, 8.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₈H₁₉FN₂O₄S₂+H+, 411.08430; found (ESI, [M+H]⁺), 411.0841.

### EXAMPLE 665

### 2-methyl-N-(3-oxo-3-pyrrolidin-1-ylpropyl)-5-(phenylsulfonyl)benzenesulfonamide

To a solution of methyl N-{[2-methyl-5-(phenylsulfonyl)phenyl]sulfonyl}-beta-alaninate (1.3 g, 3.3 mmol) in water (10 mL) and tetrahydrofuran (10 mL) was added lithium hydroxide monohydrate (0.67 g, 16 mmol). The resulting mixture was stirred overnight at room temperature. The mixture was then washed with ether and the organic phase was discarded. The aqueous phase was treated with 4 N HCl to pH 1 and then extracted with ethyl acetate. The ethyl acetate extract was dried (MgSO₄), filtered, and concentrated to provide the desired 3-(5-benzenesulfonyl-2-methyl-benzenesulfonylamino)-propionic acid (1.2 g, 3.1 mmol). The 3-(5-benzenesulfonyl-2-methyl-benzenesulfonylamino)-propionic acid (1.2 g, 3.1 mmol) was dissolved in dichloromethane (40 mL) containing dimethylformamide (3 drops) and then treated with oxalyl chloride (1.0 mL, 11 mmol). The resulting solution was stirred at room temperature for 2 h and then concentrated to dryness. The crude 3-(5-benzenesulfonyl-2-methyl-benzenesulfonylamino)-propionyl chloride was dissolved in dichloromethane and used without further purification. To a solution of crude 3-(5-benzenesulfonyl-2-methyl-benzenesulfonylamino)-propionyl chloride (0.10 g, 0.25 mmol) in dichloromethane (1 mL) was added pyrrolidine (22 mg, 0.31 mmol) and triethylamine (55 μL, 0.39 mmol). The resulting solution was stirred overnight at ambient temperature, and then diluted with ethyl acetate and washed with saturated aqueous ammonium chloride, sodium bicarbonate, and brine. The organic phase was dried (MgSO₄), concentrated, and purified by flash column chromatography to provide 2-methyl-N-(3-oxo-3-pyrrolidin-1-ylpropyl)-5-(phenylsulfonyl)benzenesulfonamide (48 mg).

MS (ES+) m/z 437.1;

HPLC purity 91.3% at 210-370 nm, 8.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 666

### N-(tert-butyl)-N³-{[2-methyl-5-(phenylsulfonyl)phenyl]sulfonyl}-β-alaninamide

In an analogous manner to Example 665, crude 3-(5-benzenesulfonyl-2-methyl-benzenesulfonylamino)-propionyl chloride (0.10 g, 0.25 mmol), t-butylamine (23 mg, 0.31 mmol) and triethylamine (55 μL, 0.39 mmol) in dichloromethane (1 mL) were used to prepare N-(tert-butyl)-N³-{[2-methyl-5-(phenylsulfonyl)phenyl]sulfonyl}-β-alaninamide (21 mg).

MS (ES+) m/z 439.1;

HPLC purity 86.3% at 210-370 nm, 8.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 667

### N³-{[2-methyl-5-(phenylsulfonyl)phenyl]sulfonyl}-N-(1,2,3,4-tetrahydronaphthalen-1-yl)-β-alaninamide

In an analogous manner to Example 665, crude 3-(5-benzenesulfonyl-2-methyl-benzenesulfonylamino)-propionyl chloride (0.10 g, 0.25 mmol), 1,2,3,4-tetrahydro-1-naphthylamine (46 mg, 0.31 mmol) and triethylamine (55 μL, 0.39 mmol) in dichloromethane (1 mL) were used to prepare N³-{[2-methyl-5-(phenylsulfonyl)phenyl]sulfonyl}-N-(1,2,3,4-tetrahydronaphthalen-1-yl)-β-alaninamide (63 mg).

MS (ES−) m/z 511.0;

HPLC purity 87.0% at 210-370 nm, 9.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 668

### N-methyl-N³-{[2-methyl-5-(phenylsulfonyl)phenyl]sulfonyl}-N-phenyl-β-alaninamide

In an analogous manner to Example 665, crude 3-(5-benzenesulfonyl-2-methyl-benzenesulfonylamino)-propionyl chloride (0.10 g, 0.25 mmol), N-methylaniline (34 mg, 0.31 mmol) and triethylamine (55 μL, 0.39 mmol) in dichloromethane (1 mL) were used to prepare N-methyl-N³-{[2-methyl-5-(phenylsulfonyl)phenyl]sulfonyl}-N-phenyl-β-alaninamide (34 mg).

MS (ES+) m/z 473.1;

HPLC purity 82.7% at 210-370 nm, 8.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 669

### 2-methyl-N-[3-(6-methyl-3,4-dihydroquinolin-1(2H)-yl)-3-oxopropyl]-5-(phenylsulfonyl)benzenesulfonamide

In an analogous manner to Example 665, crude 3-(5-benzenesulfonyl-2-methyl-benzenesulfonylamino)-propionyl chloride (0.10 g, 0.25 mmol), 6-methyl-1,2,3,4-tetrahydroquinoline (46 mg, 0.31 mmol) and triethylamine (55 μL, 0.39 mmol) in dichloromethane (1 mL) were used to prepare 2-methyl-N-[3-(6-methyl-3,4-dihydroquinolin-1(2H)-yl)-3-oxopropyl]-5-(phenylsulfonyl)benzenesulfonamide (29 mg).

MS (ES+) m/z 513.1;

HPLC purity 91.0% at 210-370 nm, 9.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 670

### 2-isopropyl-5-(phenylsulfonyl)-N-(tetrahydro-2H-pyran-4-yl)benzenesulfonamide

In an analogous manner to Step 3, Example 295:

5-Benzenesulfonyl-2-isopropyl-benzenesulfonyl chloride and tetrahydro-pyran-4-ylamine were used to prepare 2-isopropyl-5-(phenylsulfonyl)-N-(tetrahydro-2H-pyran-4-yl)benzenesulfonamide

MS (ES−) m/z 422.1;

HPLC purity 99.5% at 210-370 nm, 8.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₀H₂₅NO₅S₂+H+, 424.12469; found (ESI, [M+H]⁺), 424.127.

### EXAMPLE 671

### 2-isopropyl-5-(phenylsulfonyl)-N-(2-pyridin-2-ylethyl)benzenesulfonamide

In an analogous manner to Step 3, Example 295:

5-Benzenesulfonyl-2-isopropyl-benzenesulfonyl chloride and 2-pyridin-2-yl-ethylamine were used to prepare 2-isopropyl-5-(phenylsulfonyl)-N-(2-pyridin-2-ylethyl)benzenesulfonamide.

MS (ES+) m/z 445.0;

HPLC purity 99.5% at 210-370 nm, 9.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₂H₂₄N₂O₄S₂+H+, 445.12502; found (ESI, [M+H]⁺), 445.1251.

### EXAMPLE 672

### 2-isopropyl-5-(phenylsulfonyl)-N-(2-pyridin-3-ylethyl)benzenesulfonamide

In an analogous manner to Step 3, Example 295:

5-Benzenesulfonyl-2-isopropyl-benzenesulfonyl chloride and 2-pyridin-3-yl-ethylamine were used to prepare 2-isopropyl-5-(phenylsulfonyl)-N-(2-pyridin-3-ylethyl)benzenesulfonamide.

MS (ES+) m/z 445.0;

HPLC purity 99.6% at 210-370 nm, 8.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 673

### 2-chloro-N-(2-hydroxyethyl)-5-(phenylsulfonyl)benzenesulfonamide

In an analogous manner to example 654, 2-chloro-5-(phenylsulfonyl)-benzenesulfonyl chloride and ethanolamine were used to prepare 2-chloro-N-(2-hydroxyethyl)-5-(phenylsulfonyl)benzenesulfonamide.

MS (ES−) m/z 373.9;

HPLC purity 99.6% at 210-370 nm, 7.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₄H₁₄ClNO₅S₂+H+, 376.00747; found (ESI, [M+H]⁺), 376.0079.

### EXAMPLE 674

### 2-chloro-N-(2-hydroxy-1-methylethyl)-5-(phenylsulfonyl)benzenesulfonamide

In an analogous manner to example 654, 2-chloro-5-(phenylsulfonyl)-benzenesulfonyl chloride and alanol were used to prepare 2-chloro-N-(2-hydroxy-1-methylethyl)-5-(phenylsulfonyl)benzenesulfonamide.

MS (ES−) m/z 388.0;

HPLC purity 96.9% at 210-370 nm, 7.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₅H₁₆ClNO₅S₂+H+, 390.02312; found (ESI-FTMS, [M+H]¹⁺), 390.023

### EXAMPLE 675

### 2-chloro-N-[2-hydroxy-1-(hydroxymethyl)ethyl]-5-(phenylsulfonyl)benzenesulfonamide

In an analogous manner to example 654, 2-chloro-5-(phenylsulfonyl)-benzenesulfonyl chloride and serinol were used to prepare 2-chloro-N-[2-hydroxy-1-(hydroxymethyl)ethyl]-5-(phenylsulfonyl)benzenesulfonamide.

MS (ES−) m/z 403.9;

HPLC purity 95.9% at 210-370 nm, 6.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₅H₁₆ClNO₆S₂+H+, 406.01803; found ESI-FTMS, [M+H]¹⁺), 406.01897.

### EXAMPLE 676

### 2-chloro-N-[(1R*,2R*)-2-hydroxy-1-methyl-2-phenylethyl]-5-(phenylsulfonyl)benzenesulfonamide

In an analogous manner to example 654, 2-chloro-5-(phenylsulfonyl)-benzenesulfonyl chloride and norephedrine were used to prepare 2-chloro-N-[(1R*,2R*)-2-hydroxy-1-methyl-2-phenylethyl]-5-(phenylsulfonyl)benzenesulfonamide.

MS (ES−) m/z 463.9;

HPLC purity 100% at 210-370 nm, 9.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₁H₂₀ClNO₅S₂+Na+, 488.03636; found (ESI-FTMS, [M+Na]¹⁺), 488.0366.

### EXAMPLE 677

### 5-(phenylsulfonyl)-N-(2-pyridin-3-ylethyl)-2-(trifluoromethyl)benzenesulfonamide

Step 1: To a stirred solution of aluminum chloride (9.56 g, 71.7 mmol) in benzene (30 mL) was added 4-chloro-3-nitrobenzenesulfonyl chloride (15.3 g, 59.75 mmol). The resulting mixture was stirred overnight at room temperature, poured over ice, and extracted with ethyl acetate. The organic layer was dried over magnesium sulfate and concentrated to give 5-(phenylsulfonyl)-2-chloro-nitrobenzene. (14.9 g, 84%).

Step 2: To a stirred solution of 5-(phenylsulfonyl)-2-chloro-nitrobenzene (13.8 g, 41.66 mmol) in dimethylacetamide (85 mL) was added copper powder (15.9 g, 250 mmol), activated charcoal (6.9 g), and difluorodibromomethane (7.6 mL, 83.32 mmol). The resulting solution was stirred at 100° C. for 3 hours. The mixture was allowed to cool to room temperature and filtered through celite with ethyl acetate. The filtrate was partitioned between ammonium chloride solution (sat) and ethyl acetate. The aqueous layer was extracted a second time with ethyl acetate. The combined organic layers were washed with water several times, washed with brine, dried over magnesium sulfate and concentrated. Flash column separation using 0%-20% ethyl acetate/hexane gradient gave 5-(phenylsulfonyl)-2-trifluoromethyl-nitrobenzene (5.6 g, 41%).

Step 3: To a stirred solution of 5-(phenylsulfonyl)-2-trifluoromethyl-nitrobenzene (4.14 g, 12.5 mmol) in methanol (90 mL) was added water (4 mL) and Tin (II) Chloride (11.85 g, 62.85 mmol). The resulting solution was heated to 70° C. for 3 days. The solution was allowed to cool to room temperature and quenched with sodium bicarbonate solution (sat) and extracted several times with ethyl acetate. The combined organic layer was washed with brine, dried over magnesium sulfate and concentrated to give 5-(phenylsulfonyl)-2-trifluoromethylaniline (3.6 g, 95%).

Step 4: To a stirred solution of 5-(phenylsulfonyl)-2-trifluoromethylaniline (3.6 g, 11.95 mmol) in acetonitrile (85 mL) at 0° C. was added concentrated acetic acid (8.5 mL) and concentrated hydrochloric acid (8.5 mL). To this solution was added sodium nitrite (0.99 g, 14.34 mmol) dissolved in water (1.5 mL) in a dropwise fashion. The resulting solution was stirred for 20 minutes, followed by bubbling in sulfur dioxide over 10 minutes. Immediately upon completion of sulfur dioxide addition, copper (II) chloride dihydride (2.0 g, 12.0 mmol) dissolved in water (2 mL) was added all at once. The mixture was allowed to warm to room temperature and stirred overnight. The reaction was diluted with water and extracted with ethyl acetate several times. The combined organic layer was washed with ammonium chloride solution (sat.), water, and brine. The organic phase was dried over magnesium sulfate, and concentrated. Flash column separation using 10%-30% ethyl acetate/hexane gradient gave 2-trifluoromethyl-5-(phenylsulfonyl)-benzenesulfonyl chloride (1.7 g, 37%).

Step 5: Following the same procedure described on example 435, 2-trifluoromethyl-5-(phenylsulfonyl)-benzenesulfonyl chloride and 3-(2-aminoethyl)pyridine were used to prepare 5-(phenylsulfonyl)-N-(2-pyridin-3-ylethyl)-2-(trifluoromethyl)benzenesulfonamide.

MS (ES−) m/z 468.9;

HPLC purity 100% at 210-370 nm, 8.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₀H₁₇F₃N₂O₄S₂+H+, 471.06546; found (ESI-FTMS, [M+H]¹⁺), 471.06448.

### EXAMPLE 678

### 1-Methoxy-4-(phenylsulfonyl)benzene

Step a

A stirred solution of benzene (10 mL, 100 mmol) and 4-methoxybenzenesulfonyl chloride (4.12 g, 20 mmol) was cooled to −40° C. and treated slowly under nitrogen with solid anhydrous aluminum chloride (3.2 g, 24 mmol). After stirring neat for 4 hours at room temperature, the mixture was slowly poured into cold 1N hydrochloric acid and extracted with ethyl acetate (2×). The organic phase was washed with a saturated, aqueous sodium chloride solution, dried over anhydrous sodium sulfate, and filtered through a short column of silica gel. The filtrate was evaporated in vacuo to yield 1-methoxy-4-(phenylsulfonyl)benzene (4.96 g, 100%) as a homogeneous oil which solidified on standing.

Step b

### 2-Methoxy-5-(phenylsulfonyl)benzenesulfonyl chloride

1-methoxy-4-(phenylsulfonyl)benzene (4.96 g, 20 mmol) was stirred for 30 minutes under nitrogen with chlorosulfonic acid (13.2 mL, 23.2 g, 200 mmol). The mixture was cooled to room temperature, poured slowly into a cold solution of 1N hydrochloric acid, and extracted with ethyl acetate (2×). The organic phase was washed with a saturated, aqueous sodium chloride solution, dried over anhydrous sodium sulfate, filtered, and the solvent concentrated in vacuo to a crude oil. The crude oil was crystallized from diethyl ether-hexane to afford 2-methoxy-5-(phenylsulfonyl)benzenesulfonyl chloride (5.12 g, 74%) as a colorless solid, which was utilized in subsequent reactions.

Step c

### 2-Methoxy-5-(phenylsulfonyl)-N-(2-pyridin-3-ylethyl)benzenesulfonamide

A stirred solution of 2-methoxy-5-(phenylsulfonyl)benzenesulfonyl chloride (0.35 g, 1.0 mmol) in dichloromethane (10 mL) was treated dropwise under nitrogen with a solution of 2-(pyridin-3-yl)ethanamine (0.24 g, 2.0 mmol) in dichloromethane. The reaction was stirred for 18 hours at room temperature. The crude product was purified by preparative liquid chromatography on a Biotage® 40 Si column of pre-packed silica gel (90 g), eluting with a gradient of 50%-100% methyl tert-butyl ether in hexane at a flow rate of 50 mL/min, to afford, after crystallization from ethyl acetate-diethyl ether-hexane, 2-methoxy-5-(phenylsulfonyl)-N-(2-pyridin-3-ylethyl)benzenesulfonamide (0.26 g, 60%), as a homogeneous, colorless, crystalline solid, m.p. 181-183° C.;

MS (+ESI), m/z: 433.1 [M+H]⁺;

HRMS: calcd for C₂₀H₂₀N₂O₅S₂+H+, 433.08864; found (ESI, [M+H]⁺), 433.0907;

HPLC purity 100% at 210-370 nm, 6.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 679

### N-(2-cyanoethyl)-5-(phenylsulfonyl)-2-(trifluoromethyl)benzenesulfonamide

In an analogous manner to example 435, 2-trifluoromethyl-5-(phenylsulfonyl)-benzenesulfonyl chloride and aminopropionitrile were used to prepare N-(2-cyanoethyl)-5-(phenylsulfonyl)-2-(trifluoromethyl)benzenesulfonamide.

MS (ES−) m/z 417.0;

HPLC purity 95.3% at 210-370 nm, 8.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₆H₁₃F₃N₂O₄S₂+H+, 419.03416; found (ESI, [M+H]⁺), 419.0344.

### EXAMPLE 680

### 5-(phenylsulfonyl)-2-(trifluoromethyl)-N-{1-[4-(trifluoromethyl)benzoyl]piperidin-4-yl}benzenesulfonamide

Step 1: In an analogous manner to example 462, piperidin-4-yl-carbamic acid tert-butyl ester and 4-trifluoromethylbenzoyl chloride were used to prepare [1-(4-trifluoromethyl-benzoyl)-piperidin-4-yl]-carbamic acid tert-butyl ester.

Step 2: [1-(4-trifluoromethyl-benzoyl)-piperidin-4-yl]-carbamic acid tert-butyl ester (1.50 g, 4.0 mmol) was dissolved in HCl in 1,4-dioxane 4M solution (6 mL, 24 mmol). The resulting solution was stirred 2 hours and concentrated. The crude was partitioned between sodium bicarbonate solution (sat) and ethyl acetate. The organic layer was washed with brine, dried over magnesium sulfate and concentrated to give 1-(trifluoromethyl)benzoyl-4-aminopiperidine.

Step 3: In an analogous manner to example 435, 2-trifluoromethyl-5-(phenylsulfonyl)-benzenesulfonyl chloride and 1-(trifluoromethyl)benzoyl-4-aminopiperidine were used to prepare 5-(phenylsulfonyl)-2-(trifluoromethyl)-N-{1-[4-(trifluoromethyl)benzoyl]piperidin-4-yl}benzenesulfonamide.

MS (ES+) m/z 621.1;

HPLC purity 100% at 210-370 nm, 10.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₆H₂₂F₆N₂O₅S₂+H+, 621.09471; found (ESI, [M+H]⁺), 621.0933.

### EXAMPLE 681

### N-(2-hydroxyethyl)-5-(phenylsulfonyl)-2-(trifluoromethyl)benzenesulfonamide

In an analogous manner to example 654, 2-trifluoromethyl-5-(phenylsulfonyl)-benzenesulfonyl chloride and ethanolamine were used to prepare N-(2-hydroxyethyl)-5-(phenylsulfonyl)-2-(trifluoromethyl)benzenesulfonamide.

MS (ES−) m/z 410.1;

HPLC purity 100% at 210-370 nm, 7.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₅H₁₄F₃NO₅S₂+H+, 410.03382; found (ESI, [M+H]⁺), 410.0352.

### EXAMPLE 682

### N-(2-hydroxy-2-phenylethyl)-5-(phenylsulfonyl)-2-(trifluoromethyl)benzenesulfonamide

In an analogous manner to example 654, 2-trifluoromethyl-5-(phenylsulfonyl)-benzenesulfonyl chloride and 2-phenylethanolamine were used to prepare N-(2-hydroxy-2-phenylethyl)-5-(phenylsulfonyl)-2-(trifluoromethyl)benzenesulfonamide.

MS (ES−) m/z 484.0;

HPLC purity 100% at 210-370 nm, 9.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 683

### N-(2-hydroxy-1-methylethyl)-5-(phenylsulfonyl)-2-(trifluoromethyl)benzenesulfonamide

In an analogous manner to example 654, 2-trifluoromethyl-5-(phenylsulfonyl)-benzenesulfonyl chloride and alanol were used to prepare N-(2-hydroxy-1-methylethyl)-5-(phenylsulfonyl)-2-(trifluoromethyl)benzenesulfonamide.

MS (ES−) m/z 422.0;

HPLC purity 97.9% at 210-370 nm, 8.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₆H₁₆F₃NO₅S₂+H+, 424.04947; found (ESI, [M+H]⁺), 424.0484.

### EXAMPLE 684

### N-[(1R*,2R*)-2-hydroxy-1-methyl-2-phenylethyl]-5-(phenylsulfonyl)-2-(trifluoromethyl)benzenesulfonamide

In an analogous manner to example 654, 2-trifluoromethyl-5-(phenylsulfonyl)-benzenesulfonyl chloride and norephedrine were used to prepare N-[(1R*,2R*)-2-hydroxy-1-methyl-2-phenylethyl]-5-(phenylsulfonyl)-2-(trifluoromethyl)benzenesulfonamide.

MS (ES−) m/z 498.0;

HPLC purity 98.0% at 210-370 nm, 9.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 685

### N-[(1S,2R)-2-hydroxy-1-methyl-2-phenylethyl]-5-(phenylsulfonyl)-2-(trifluoromethyl)benzenesulfonamide

In an analogous manner to example 654, 2-trifluoromethyl-5-(phenylsulfonyl)-benzenesulfonyl chloride and (1S,2R)-2-hydroxy-2-phenyl-1-methyl-1-aminoethane were used to prepare N-[(1S,2R)-2-hydroxy-1-methyl-2-phenylethyl]-5-(phenylsulfonyl)-2-(trifluoromethyl)benzenesulfonamide.

MS (ES−) m/z 498.0;

HPLC purity 96.6% at 210-370 nm, 9.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 686

### N-[(1R,2S)-2-hydroxy-1-methyl-2-phenylethyl]-5-(phenylsulfonyl)-2-(trifluoromethyl)benzenesulfonamide

In an analogous manner to example 654, 2-trifluoromethyl-5-(phenylsulfonyl)-benzenesulfonyl chloride and (1R,2S)-2-hydroxy-2-phenyl-1-methyl-1-aminoethane were used to prepare N-[(1R,2S)-2-hydroxy-1-methyl-2-phenylethyl]-5-(phenylsulfonyl)-2-(trifluoromethyl)benzenesulfonamide.

MS (ES−) m/z 498.0;

HPLC purity 97.6% at 210-370 nm, 9.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 687

### tert-butyl 4-({[5-(phenylsulfonyl)-2-(trifluoromethyl)phenyl]sulfonyl}amino)piperidine-1-carboxylate

In an analogous manner to example 435, 2-trifluoromethyl-5-(phenylsulfonyl)-benzenesulfonyl chloride and 4-aminopiperidine-1-carboxylic acid tert-butyl ester were used to prepare tert-butyl 4-({[5-(phenylsulfonyl)-2-(trifluoromethyl)phenyl]sulfonyl}amino)piperidine-1-carboxylate.

MS (ES−) m/z 547.0;

HPLC purity 97.4% at 210-370 nm, 10.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 688

### 5-(phenylsulfonyl)-N-piperidin-4-yl-2-(trifluoromethyl)benzenesulfonamide

tert-butyl 4-({[5-(phenylsulfonyl)-2-(trifluoromethyl)phenyl]sulfonyl}amino)piperidine-1-carboxylate (1.57 g, 2.86 mmol) was dissolved in HCl in 1,4-dioxane 4M solution (8 mL, 32 mmol). The resulting solution was stirred overnight and concentrated. The crude was partitioned between sodium bicarbonate solution (sat) and ethyl acetate. The organic layer was washed with brine, dried over magnesium sulfate and concentrated to give 5-(phenylsulfonyl)-N-piperidin-4-yl-2-(trifluoromethyl)benzenesulfonamide (1.1 g, 85%).

MS (ES+) m/z 449.1;

HPLC purity 98.0% at 210-370 nm, 6.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₈H₁₉F₃N₂O₄S₂+H+, 449.08111; found (ESI, [M+H]⁺), 449.083.

### EXAMPLE 689

### 2-Methoxy-5-(phenylsulfonyl)-N-(tetrahydro-2H-pyran-4-yl)benzenesulfonamide

A stirred solution of 2-methoxy-5-(phenylsulfonyl)benzenesulfonyl chloride (0.35 g, 1.0 mmol) in dichloromethane (10 mL) was treated under nitrogen with tetrahydro-2H-pyran-4-amine hydrochloride (0.27 g, 2.0 mmol) and a solution of diisopropylethylamine (0.39 g, 3.0 mmol) in dichloromethane. The reaction was stirred for 18 hours at room temperature. The crude product was purified by preparative liquid chromatography on a Biotage® 40 Si column of pre-packed silica gel (90 g), eluting with a gradient of 30%-70% methyl tert-butyl ether in hexane at a flow rate of 50 mL/min, to afford, after crystallization from ethyl acetate-diethyl ether-hexane, 2-methoxy-5-(phenylsulfonyl)-N-(tetrahydro-2H-pyran-4-yl)benzenesulfonamide (0.29 g, 70%), as a homogeneous, colorless, crystalline solid, m.p. 176-178° C.;

MS (+ESI), m/z: 412.1 [M+H]⁺;

HRMS: calcd for C₁₈H₂₁NO₆S₂+H+, 412.08830; found (ESI, [M+H]⁺), 412.0899;

HPLC purity 100% at 210-370 nm, 7.5 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 690

### 4-[({5-[(4-fluorophenyl)sulfonyl]-2-isopropylphenyl}sulfonyl)amino]-N-1-naphthylpiperidine-1-carbothioamide

To a solution of 5-[(4-fluorophenyl)sulfonyl]-2-isopropyl-N-piperidin-4-ylbenzenesulfonamide (example 520) (75 mg, 0.17 mmol) in dichloromethane (4.0 mL) was added triethylamine (36 μL, 0.25 mmol) followed by 1-naphthyl isothiocyanate (37 mg, 0.2 mmol). The reaction was mixed on an orbital shaker overnight at room temperature. The following day the reaction was concentrated down onto silica gel and purified using automated chromatography with a gradient mobile phase consisting of ethyl acetate and hexane resulting in the isolation of 4-[({5-[(4-fluorophenyl)sulfonyl]-2-isopropylphenyl}sulfonyl)amino]-N-1-naphthylpiperidine-1-carbothioamide (42 mg, 40%).

MS (ES+) m/z 626;

HRMS: calcd. for C₃₁H₃₂FN₃O₄S₃+H⁺, 626.1617: found (ESI, [m+H]⁺), 626.1589.

### EXAMPLE 691

### N-(2-fluorophenyl)-4-[({5-[(4-fluorophenyl)sulfonyl]-2-isopropylphenyl}sulfonyl)amino]piperidine-1-carbothioamide

In an analogous manner to example 690, 5-[(4-fluorophenyl)sulfonyl]-2-isopropyl-N-piperidin-4-ylbenzenesulfonamide and 2-fluorophenyl isothiocyanate were used to prepare N-(2-fluorophenyl)-4-[({5-[(4-fluorophenyl)sulfonyl]-2-isopropylphenyl}sulfonyl)amino]piperidine-1-carbothioamide.

MS (ES+) m/z 594.1;

HRMS: calcd. for C₂₇H₂₉F₂N₃O₄S₃+H⁺, 594.1366: found (ESI, [m+H]⁺), 594.137.

### EXAMPLE 692

### 4-[({5-[(4-fluorophenyl)sulfonyl]-2-isopropylphenyl}sulfonyl)amino]-N-(2-methylphenyl)piperidine-1-carbothioamide

In an analogous manner to example 690, 5-[(4-fluorophenyl)sulfonyl]-2-isopropyl-N-piperidin-4-ylbenzenesulfonamide and o-toluyl isothiocyanate were used to prepare 4-[({5-[(4-fluorophenyl)sulfonyl]-2-isopropylphenyl}sulfonyl)amino]-N-(2-methylphenyl)piperidine-1-carbothioamide.

MS (ES+) m/z 590;

HRMS: calcd. for C₂₈H₃₂FN₃O₄S₃+H⁺, 590.1617: found (ESI, [m+H]⁺), 590.1628.

### EXAMPLE 693

### ethyl({4-[({5-[(4-fluorophenyl)sulfonyl]-2-isopropylphenyl}sulfonyl)amino]piperidin-1-yl}carbonothioyl)carbamate

In an analogous manner to example 690, 5-[(4-fluorophenyl)sulfonyl]-2-isopropyl-N-piperidin-4-ylbenzenesulfonamide and ethyl isothiocyanatoformate were used to prepare ethyl({4-[({5-[(4-fluorophenyl)sulfonyl]-2-isopropylphenyl}sulfonyl)amino]piperidine-1-yl}carbonothioyl)carbamate.

MS (ES+) m/z 572;

HRMS: calcd. for C₂₄H₃₀FN₃O₆S₃+H⁺, 572.1359: found (ESI, [m+H]⁺), 572.1353.

### EXAMPLE 694

### N-butyl-4-[({5-[(4-fluorophenyl)sulfonyl]-2-isopropylphenyl}sulfonyl)amino]piperidine-1-carbothioamide

In an analogous manner to example 690, 5-[(4-fluorophenyl)sulfonyl]-2-isopropyl-N-piperidin-4-ylbenzenesulfonamide and 1-butyl isothiocyanate were used to prepare N-butyl-4-[({5-[(4-fluorophenyl)sulfonyl]-2-isopropylphenyl}sulfonyl)amino]piperidine-1-carbothioamide.

MS (ES+) m/z 556.2;

HRMS: calcd. for C₂₅H₃₄FN₃O₄S₃+H⁺, 556.1773: found (ESI, [m+H]⁺), 556.1765.

### EXAMPLE 695

### 4-[({5-[(4-fluorophenyl)sulfonyl]-2-isopropylphenyl}sulfonyl)amino]-N-(4-methoxyphenyl)piperidine-1-carbothioamide

In an analogous manner to example 690, 5-[(4-fluorophenyl)sulfonyl]-2-isopropyl-N-piperidin-4-ylbenzenesulfonamide and 4-isothiocyanatoanisole were used to prepare 4-[({5-[(4-fluorophenyl)sulfonyl]-2-isopropylphenyl}sulfonyl)amino]-N-(4-methoxyphenyl)piperidine-1-carbothioamide.

MS (ES+) m/z 606.1;

HRMS: calcd. for C₂₈H₃₂FN₃O₅S₃+H⁺, 606.1566: found (ESI, [m+H]⁺), 606.1456.

### EXAMPLE 696

### methyl 4-[({4-[({5-[(4-fluorophenyl)sulfonyl]-2-isopropylphenyl}sulfonyl)amino]piperidin-1-yl}carbonothioyl)amino]benzoate

In an analogous manner to example 690, 5-[(4-fluorophenyl)sulfonyl]-2-isopropyl-N-piperidin-4-ylbenzenesulfonamide and 4-isothiocyanato-benzoic acid methyl ester were used to prepare methyl 4-[({4-[({5-[(4-fluorophenyl)sulfonyl]-2-isopropylphenyl}sulfonyl)amino]piperidin-1-yl}carbonothioyl)amino]benzoate.

MS (ES−) m/z 632.1;

HRMS: calcd. for C₂₉H₃₂FN₃O₆S₃+H⁺, 634.1516: found (ESI, [m+H]⁺), 634.1478.

### EXAMPLE 697

### methyl N-({4-[({5-[(4-fluorophenyl)sulfonyl]-2-isopropylphenyl}sulfonyl)amino]piperidin-1-yl}carbonothioyl)glycinate

In an analogous manner to example 690, 5-[(4-fluorophenyl)sulfonyl]-2-isopropyl-N-piperidin-4-ylbenzenesulfonamide and 2-isothiocyanatoacetic methyl ester were used to prepare methyl N-({4-[({5-[(4-fluorophenyl)sulfonyl]-2-isopropylphenyl}sulfonyl)amino]piperidin-1-yl}carbonothioyl)glycinate.

MS (ES−) m/z 570;

HRMS: calcd. for C₂₄H₃₀FN₃O₆S₃+H⁺, 572.1359: found (ESI, [m+H]⁺), 572.1348.

### EXAMPLE 698

### 4-[({5-[(4-fluorophenyl)sulfonyl]-2-isopropylphenyl}sulfonyl)amino]-N-(2-morpholin-4-ylethyl)piperidine-1-carbothioamide

In an analogous manner to example 690, 5-[(4-fluorophenyl)sulfonyl]-2-isopropyl-N-piperidin-4-ylbenzenesulfonamide and 2-(4-morpholino)ethyl isothiocyanate were used to prepare methyl N-({4-[({5-[(4-fluorophenyl)sulfonyl]-2-isopropylphenyl}sulfonyl)amino]piperidin-1-yl}carbonothioyl)glycinate.

MS (ES−) m/z 611.

### EXAMPLE 699

### 4-[({5-[(4-fluorophenyl)sulfonyl]-2-isopropylphenyl}sulfonyl)amino]-N-(3-nitrophenyl)piperidine-1-carbothioamide

In an analogous manner to example 690, 5-[(4-fluorophenyl)sulfonyl]-2-isopropyl-N-piperidin-4-ylbenzenesulfonamide and 3-nitrophenyl isothiocyanate were used to prepare 4-[({5-[(4-fluorophenyl)sulfonyl]-2-isopropylphenyl}sulfonyl)amino]-N-(3-nitrophenyl)piperidine-1-carbothioamide.

MS (ES−) m/z 619;

HRMS: calcd. for C₂₇H₂₉FN₄O₆S₃+H⁺, 621.1312: found (ESI, [m+H]⁺), 621.1219.

### EXAMPLE 700

### 3-[({4-[({5-[(4-fluorophenyl)sulfonyl]-2-isopropylphenyl}sulfonyl)amino]piperidin-1-yl}carbonothioyl)amino]benzoic acid

In an analogous manner to example 690, 5-[(4-fluorophenyl)sulfonyl]-2-isopropyl-N-piperidin-4-ylbenzenesulfonamide and 3-carboxyphenyl isothiocyanate were used to prepare 3-[({4-[({5-[(4-fluorophenyl)sulfonyl]-2-isopropylphenyl}sulfonyl)amino]piperidin-1-yl}carbonothioyl)amino]benzoic acid.

MS (ES−) m/z 618;

HRMS: calcd. for C₂₈H₃₀FN₃O₆S₃+H⁺, 620.1359: found (ESI, [m+H]⁺), 620.1364.

### EXAMPLE 701

### 4-[({5-[(4-fluorophenyl)sulfonyl]-2-isopropylphenyl}sulfonyl)amino]-N-pyridin-3-ylpiperidine-1-carbothioamide

In an analogous manner to example 690, 5-[(4-fluorophenyl)sulfonyl]-2-isopropyl-N-piperidin-4-ylbenzenesulfonamide and 3-pyridyl isothiocyanate were used to prepare 4-[({5-[(4-fluorophenyl)sulfonyl]-2-isopropylphenyl}sulfonyl)amino]-N-pyridin-3-ylpiperidine-1-carbothioamide.

MS (ES+) m/z 577.1;

HRMS: calcd. for C₂₆H₂₉FN₄O₄S₃+H⁺, 577.1413: found (ESI, [m+H]⁺), 577.1396.

### EXAMPLE 702

### N-[1-(2-chlorobenzoyl)piperidin-4-yl]-5-(phenylsulfonyl)-2-(trifluoromethyl)benzenesulfonamide

In an analogous manner to example 462, 5-(phenylsulfonyl)-N-piperidin-4-yl-2-(trifluoromethyl)benzenesulfonamide and 2-chlorobenzoyl chloride were used to prepare N-[1-(2-chlorobenzoyl)piperidin-4-yl]-5-(phenylsulfonyl)-2-(trifluoromethyl)benzenesulfonamide.

MS (ES+) m/z 587.0;

HPLC purity 93.6% at 210-370 nm, 9.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₅H₂₂ClF₃N₂O₅S₂+H+, 587.06835; found (ESI, [M+H]⁺), 587.0666.

### EXAMPLE 703

### N-[1-(2-methoxybenzoyl)piperidin-4-yl]-5-(phenylsulfonyl)-2-(trifluoromethyl)benzenesulfonamide

In an analogous manner to example 462, 5-(phenylsulfonyl)-N-piperidin-4-yl-2-(trifluoromethyl)benzenesulfonamide and 2-methoxybenzoyl chloride were used to prepare N-[1-(2-methoxybenzoyl)piperidin-4-yl]-5-(phenylsulfonyl)-2-(trifluoromethyl)benzenesulfonamide.

MS (ES+) m/z 583.1;

HPLC purity 96.8% at 210-370 nm, 9.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 704

### N-[1-(3-chlorobenzoyl)piperidin-4-yl]-5-(phenylsulfonyl)-2-(trifluoromethyl)benzenesulfonamide

In an analogous manner to example 462, 5-(phenylsulfonyl)-N-piperidin-4-yl-2-(trifluoromethyl)benzenesulfonamide and 3-chlorobenzoyl chloride were used to prepare N-[1-(3-chlorobenzoyl)piperidin-4-yl]-5-(phenylsulfonyl)-2-(trifluoromethyl)benzenesulfonamide.

MS (ES+) m/z 587.0;

HPLC purity 98.4% at 210-370 nm, 9.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₅H₂₂ClF₃N₂O₅S₂+H+, 587.06835; found (ESI, [M+H]⁺), 587.0674.

### EXAMPLE 705

### N-[1-(3,4-difluorobenzoyl)piperidin-4-yl]-5-(phenylsulfonyl)-2-(trifluoromethyl)benzenesulfonamide

In an analogous manner to example 462, 5-(phenylsulfonyl)-N-piperidin-4-yl-2-(trifluoromethyl)benzenesulfonamide and 3,4-difluorobenzoyl chloride were used to prepare N-[1-(3,4-difluorobenzoyl)piperidin-4-yl]-5-(phenylsulfonyl)-2-(trifluoromethyl)benzenesulfonamide.

MS (ES+) m/z 589.1;

HPLC purity 98.3% at 210-370 nm, 9.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₅H₂₁F₅N₂O₅S₂+H+, 589.08848; found (ESI, [M+H]⁺), 589.0875.

### EXAMPLE 706

### N-[1-(3,5-difluorobenzoyl)piperidin-4-yl]-5-(phenylsulfonyl)-2-(trifluoromethyl)benzenesulfonamide

In an analogous manner to example 462, 5-(phenylsulfonyl)-N-piperidin-4-yl-2-(trifluoromethyl)benzenesulfonamide and 3,5-difluorobenzoyl chloride were used to prepare N-[1-(3,5-difluorobenzoyl)piperidin-4-yl]-5-(phenylsulfonyl)-2-(trifluoromethyl)benzenesulfonamide.

MS (ES+) m/z 589.1;

HPLC purity 99.0% at 210-370 nm, 9.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₅H₂₁F₅N₂O₅S₂+H+, 589.08848; found (ESI, [M+H]⁺), 589.0884.

### EXAMPLE 707

### N-[1-(2,6-dimethoxybenzoyl)piperidin-4-yl]-5-(phenylsulfonyl)-2-(trifluoromethyl)benzenesulfonamide

In an analogous manner to example 462, 5-(phenylsulfonyl)-N-piperidin-4-yl-2-(trifluoromethyl)benzenesulfonamide and 2,6-dimethoxybenzoyl chloride were used to prepare N-[1-(2,6-dimethoxybenzoyl)piperidin-4-yl]-5-(phenylsulfonyl)-2-(trifluoromethyl)benzenesulfonamide.

MS (ES+) m/z 613.1;

HPLC purity 98.3% at 210-370 nm, 9.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₇H₂₇F₃N₂O₇S₂+H+, 613.12845; found (ESI, [M+H]⁺), 613.1266.

### EXAMPLE 708

### N-[1-(2,4-dimethoxybenzoyl)piperidin-4-yl]-5-(phenylsulfonyl)-2-(trifluoromethyl)benzenesulfonamide

In an analogous manner to example 462, 5-(phenylsulfonyl)-N-piperidin-4-yl-2-(trifluoromethyl)benzenesulfonamide and 2,4-dimethoxybenzoyl chloride were used to prepare N-[1-(2,4-dimethoxybenzoyl)piperidin-4-yl]-5-(phenylsulfonyl)-2-(trifluoromethyl)benzenesulfonamide.

MS (ES+) m/z 613.1;

HPLC purity 95.9% at 210-370 nm, 9.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 709

### 5-(phenylsulfonyl)-N-{1-[4-(trifluoromethoxy)benzoyl]piperidin-4-yl}-2-(trifluoromethyl)benzenesulfonamide

In an analogous manner to example 462, 5-(phenylsulfonyl)-N-piperidin-4-yl-2-(trifluoromethyl)benzenesulfonamide and 4-trifluoromethoxybenzoyl chloride were used to prepare 5-(phenylsulfonyl)-N-{1-[4-(trifluoromethoxy)benzoyl]piperidin-4-yl}-2-(trifluoromethyl)benzenesulfonamide.

MS (ES+) m/z 637.1;

HPLC purity 98.6% at 210-370 nm, 10.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₆H₂₂F₆N₂O₆S₂+H+, 637.08962; found (ESI, [M+H]⁺), 637.0912.

### EXAMPLE 710

### N-{1-[2-fluoro-4-(trifluoromethyl)benzoyl]piperidin-4-yl}-5-(phenylsulfonyl)-2-(trifluoromethyl)benzenesulfonamide

In an analogous manner to example 462, 5-(phenylsulfonyl)-N-piperidin-4-yl-2-(trifluoromethyl)benzenesulfonamide and 2-fluoro-4-trifluoromethylbenzoyl chloride were used to prepare N-{1-[2-fluoro-4-(trifluoromethyl)benzoyl]piperidin-4-yl}-5-(phenylsulfonyl)-2-(trifluoromethyl)benzenesulfonamide.

MS (ES+) m/z 639.1;

HPLC purity 98.9% at 210-370 nm, 10.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₆H₂₁F₇N₂O₅S₂+H+, 639.08528; found (ESI, [M+H]⁺), 639.0881.

### EXAMPLE 711

### N-{1-[3-fluoro-4-(trifluoromethyl)benzoyl]piperidin-4-yl}-5-(phenylsulfonyl)-2-(trifluoromethyl)benzenesulfonamide

In an analogous manner to example 462, 5-(phenylsulfonyl)-N-piperidin-4-yl-2-(trifluoromethyl)benzenesulfonamide and 3-fluoro-4-trifluoromethylbenzoyl chloride were used to prepare N-{1-[3-fluoro-4-(trifluoromethyl)benzoyl]piperidin-4-yl}-5-(phenylsulfonyl)-2-(trifluoromethyl)benzenesulfonamide.

MS (ES+) m/z 639.1;

HPLC purity 98.2% at 210-370 nm, 10.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₆H₂₁F₇N₂O₅S₂+H+, 639.08528; found (ESI, [M+H]⁺), 639.0863.

### EXAMPLE 712

### N-[1-(3,4-dichlorobenzoyl)piperidin-4-yl]-5-(phenylsulfonyl)-2-(trifluoromethyl)benzenesulfonamide

In an analogous manner to example 462, 5-(phenylsulfonyl)-N-piperidin-4-yl-2-(trifluoromethyl)benzenesulfonamide and 3,4-dichlorobenzoyl chloride were used to prepare N-[1-(3,4-dichlorobenzoyl)piperidin-4-yl]-5-(phenylsulfonyl)-2-(trifluoromethyl)benzenesulfonamide.

MS (ES+) m/z 621.9;

HPLC purity 98.2% at 210-370 nm, 10.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₅H₂₁Cl₂F₃N₂O₅S₂+H+, 621.02938; found (ESI, [M+H]⁺), 621.0306.

### EXAMPLE 713

### N-[1-(4-chlorobenzoyl)piperidin-4-yl]-5-(phenylsulfonyl)-2-(trifluoromethyl)benzenesulfonamide

In an analogous manner to example 462, 5-(phenylsulfonyl)-N-piperidin-4-yl-2-(trifluoromethyl)benzenesulfonamide and 4-chlorobenzoyl chloride were used to prepare N-[1-(4-chlorobenzoyl)piperidin-4-yl]-5-(phenylsulfonyl)-2-(trifluoromethyl)benzenesulfonamide.

MS (ES+) m/z 587.0;

HPLC purity 98.2% at 210-370 nm, 9.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₅H₂₂ClF₃N₂O₅S₂+H+, 587.06835; found (ESI, [M+H]⁺), 587.0698.

### EXAMPLE 714

### N-(1-isonicotinoylpiperidin-4-yl)-5-(phenylsulfonyl)-2-(trifluoromethyl)benzenesulfonamide

In an analogous manner to example 462, 5-(phenylsulfonyl)-N-piperidin-4-yl-2-(trifluoromethyl)benzenesulfonamide and pyridine-4-carbonyl chloride were used to prepare N-(1-isonicotinoylpiperidin-4-yl)-5-(phenylsulfonyl)-2-(trifluoromethyl)benzenesulfonamide.

MS (ES+) m/z 554.1;

HPLC purity 99.4% at 210-370 nm, 8.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₄H₂₂F₃N₃O₅S₂+H+, 554.10257; found (ESI, [M+H]⁺), 554.1038.

### EXAMPLE 715

### N-[1-(2-chloro-6-methoxyisonicotinoyl)piperidin-4-yl]-5-(phenylsulfonyl)-2-(trifluoromethyl)benzenesulfonamide

In an analogous manner to example 462, 5-(phenylsulfonyl)-N-piperidin-4-yl-2-(trifluoromethyl)benzenesulfonamide and 2-chloro-6-methoxypyridine-4-carbonyl chloride were used to prepare N-[1-(2-chloro-6-methoxyisonicotinoyl)piperidin-4-yl]-5-(phenylsulfonyl)-2-(trifluoromethyl)benzenesulfonamide.

MS (ES+) m/z 618.1;

HPLC purity 98.5% at 210-370 nm, 9.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₅H₂₃ClF₃N₃O₆S₂+H+, 618.07416; found (ESI, [M+H]⁺), 618.0737.

### EXAMPLE 716

### N-[1-(2-chloro-4-fluorobenzoyl)piperidin-4-yl]-5-(phenylsulfonyl)-2-(trifluoromethyl)benzenesulfonamide

In an analogous manner to example 462, 5-(phenylsulfonyl)-N-piperidin-4-yl-2-(trifluoromethyl)benzenesulfonamide and 2-chloro-4-fluorobenzoyl chloride were used to prepare N-[1-(2-chloro-4-fluorobenzoyl)piperidin-4-yl]-5-(phenylsulfonyl)-2-(trifluoromethyl)benzenesulfonamide.

MS (ES+) m/z 605.0;

HPLC purity 98.7% at 210-370 nm, 9.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₅H₂₁ClF₄N₂O₅S₂+H+, 605.05893; found (ESI, [M+H]⁺), 605.0582.

### EXAMPLE 717

### 5-(phenylsulfonyl)-N-[1-(2,4,6-trifluorobenzoyl)piperidin-4-yl]-2-(trifluoromethyl)benzenesulfonamide

In an analogous manner to example 462, 5-(phenylsulfonyl)-N-piperidin-4-yl-2-(trifluoromethyl)benzenesulfonamide and 2,4,6-trifluorobenzoyl chloride were used to prepare 5-(phenylsulfonyl)-N-[1-(2,4,6-trifluorobenzoyl)piperidin-4-yl]-2-(trifluoromethyl)benzenesulfonamide.

MS (ES+) m/z 607.0;

HPLC purity 93.5% at 210-370 nm, 9.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₅H₂₀F₆N₂O₅S₂+H+, 607.07906; found (ESI, [M+H]⁺), 607.0799.

### EXAMPLE 718

### N-[1-(4-tert-butylbenzoyl)piperidin-4-yl]-5-(phenylsulfonyl)-2-(trifluoromethyl)benzenesulfonamide

In an analogous manner to example 462, 5-(phenylsulfonyl)-N-piperidin-4-yl-2-(trifluoromethyl)benzenesulfonamide and 4-tert-butylbenzoyl chloride were used to prepare N-[1-(4-tert-butylbenzoyl)piperidin-4-yl]-5-(phenylsulfonyl)-2-(trifluoromethyl)benzenesulfonamide.

MS (ES+) m/z 609.1;

HPLC purity 98.0% at 210-370 nm, 10.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₉H₃₁F₃N₂O₅S₂+H+, 609.16992; found (ESI, [M+H]⁺), 609.1714.

### EXAMPLE 719

### N-(4-{[4-({[5-(phenylsulfonyl)-2-(trifluoromethyl)phenyl]sulfonyl}amino)piperidin-1-yl]carbonyl}phenyl)acetamide

In an analogous manner to example 462, 5-(phenylsulfonyl)-N-piperidin-4-yl-2-(trifluoromethyl)benzenesulfonamide and 4-N-acetamidebenzoyl chloride were used to prepare N-(4-{[4-({[5-(phenylsulfonyl)-2-(trifluoromethyl)phenyl]sulfonyl}amino)piperidin-1-yl]carbonyl}phenyl)acetamide.

MS (ES+) m/z 610.1;

HPLC purity 96.7% at 210-370 nm, 8.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₇H₂₆F₃N₃O₆S₂+H+, 610.12879; found (ESI, [M+H]⁺), 610.1293.

### EXAMPLE 720

### N-[(1S)-1-benzyl-2-hydroxyethyl]-5-(phenylsulfonyl)-2-(trifluoromethyl)benzenesulfonamide

In an analogous manner to example 435, 2-trifluoromethyl-5-(phenylsulfonyl)-benzenesulfonyl chloride and L-phenylalaninol were used to prepare N-[(1S)-1-benzyl-2-hydroxyethyl]-5-(phenylsulfonyl)-2-(trifluoromethyl)benzenesulfonamide.

MS (ES+) m/z 500.0;

HPLC purity 99.1% at 210-370 nm, 9.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₂H₂₀F₃NO₅S₂+H+, 500.08077; found (ESI, [M+H]⁺), 500.0791.

### EXAMPLE 721

### N-[(1S)-2-hydroxy-1-(1H-indol-3-ylmethyl)ethyl]-5-(phenylsulfonyl)-2-(trifluoromethyl)benzenesulfonamide

In an analogous manner to example 654, 2-trifluoromethyl-5-(phenylsulfonyl)-benzenesulfonyl chloride and L-tryptophanol were used to prepare N-[(1S)-2-hydroxy-1-(1H-indol-3-ylmethyl)ethyl]-5-(phenylsulfonyl)-2-(trifluoromethyl)benzenesulfonamide.

MS (ES+) m/z 539.0;

HPLC purity 97.2% at 210-370 nm, 9.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₄H₂₁F₃N₂O₅S₂+H+, 539.09167; found (ESI, [M+H]⁺), 539.0943.

### EXAMPLE 722

### N-[1-(2-hydroxy-2-methylpropyl)piperidin-4-yl]-5-(phenylsulfonyl)-2-(trifluoromethyl)benzenesulfonamide

To a stirred mixture of 5-(phenylsulfonyl)-N-piperidin-4-yl-2-(trifluoromethyl)benzenesulfonamide (0.075 g, 0.167 mmol) in ethanol (2 mL) was added 2,2-Dimethyl-oxirane (0.05 g, 0.69 mmol) and the resulting mixture was heated to reflux overnight. The crude mixture was concentrated and flash column separation using 50% to 100% ethyl acetate/hexane gradient gave N-[1-(2-hydroxy-2-methylpropyl)piperidin-4-yl]-5-(phenylsulfonyl)-2-(trifluoromethyl)benzenesulfonamide (0.06 g, 70%).

MS (ES−) m/z 519.0;

HPLC purity 96.8% at 210-370 nm, 7.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₂H₂₇F₃N₂O₅S₂+H+, 521.13862; found (ESI, [M+H]⁺), 521.1403.

### EXAMPLE 723

### N-[2-(1-oxidopyridin-3-yl)ethyl]-5-(phenylsulfonyl)-2-(trifluoromethyl)benzenesulfonamide

To a stirred solution of 5-(phenylsulfonyl)-N-(2-pyridin-3-ylethyl)-2-(trifluoromethyl)benzenesulfonamide (0.10 g, 0.21 mmol) in concentrated acetic acid (2 mL) was added 30% hydrogen peroxide solution (2 mL) and the resulting solution was heated to reflux for three hours. The solution was allowed to cool, poured into sodium bicarbonate solution (sat), and extracted with ethyl acetate. The organic layer was washed with sodium dithionite solution (sat), and brine. The organic layer was concentrated and flash column separation using 0%-10% methanol/methylene chloride gradient gave N-[2-(1-oxidopyridin-3-yl)ethyl]-5-(phenylsulfonyl)-2-(trifluoromethyl)benzenesulfonamide (0.015 g, 15%).

MS (ES−) m/z 485.0;

HPLC purity 98.8% at 210-370 nm, 7.5 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₀H₁₇F₃N₂O₅S₂+H+, 487.06037; found (ESI, [M+H]⁺), 487.0619.

### EXAMPLE 724

### 5-(phenylsulfonyl)-2-(trifluoromethyl)-N-(1-{[4-(trifluoromethyl)phenyl]carbonothioyl}piperidin-4-yl)benzenesulfonamide

To a stirred solution of 5-(phenylsulfonyl)-2-(trifluoromethyl)-N-{1-[4-(trifluoromethyl)benzoyl]piperidin-4-yl}benzenesulfonamide (0.10 g, 0.16 mmol) in toluene (1 mL) was added Lawesson's reagent (0.06 g, 0.15 mmol) and the resulting solution was stirred overnight at reflux. The solution was allowed to cool, partitioned between ethyl acetate and ammonium chloride solution (sat.). The organic layer was concentrated and flash column separation using 0% to 20% ethyl acetate/hexane gradient gave 5-(phenylsulfonyl)-2-(trifluoromethyl)-N-(1-{[4-(trifluoromethyl)phenyl]carbonothioyl}piperidin-4-yl)benzenesulfonamide (0.053 g, 56%).

MS (ES+) m/z 637.1;

HPLC purity 100% at 210-370 nm, 10.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₆H₂₂F₆N₂O₄S₃+H+, 637.07186; found (ESI, [M+H]⁺), 637.0749.

### EXAMPLE 725

### N-[(1S)-2-hydroxy-1-methylethyl]-5-(phenylsulfonyl)-2-(trifluoromethyl)benzenesulfonamide

In an analogous manner to example 654, 2-trifluoromethyl-5-(phenylsulfonyl)-benzenesulfonyl chloride and S-2-amino-1-propanol were used to prepare N-[(1S)-2-hydroxy-1-methylethyl]-5-(phenylsulfonyl)-2-(trifluoromethyl)benzenesulfonamide.

MS (ES+) m/z 424.1;

HPLC purity 95.1% at 210-370 nm, 8.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₆H₁₆F₃NO₅S₂+H+, 424.04947; found (ESI, [M+H]⁺), 424.0497.

### EXAMPLE 726

### N-[(1R)-2-hydroxy-1-methylethyl]-5-(phenylsulfonyl)-2-(trifluoromethyl)benzenesulfonamide

In an analogous manner to example 654, 2-trifluoromethyl-5-(phenylsulfonyl)-benzenesulfonyl chloride and R-2-amino-1-propanol were used to prepare N-[(1R)-2-hydroxy-1-methylethyl]-5-(phenylsulfonyl)-2-(trifluoromethyl)benzenesulfonamide.

MS (ES+) m/z 424.0;

HPLC purity 98.2% at 210-370 nm, 8.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₆H₁₆F₃NO₅S₂+H+, 424.04947; found (ESI, [M+H]⁺), 424.0505.

### EXAMPLE 727

### 5-[(4-fluorophenyl)sulfonyl]-N-(2-hydroxy-2-pyridin-2-ylethyl)-2-isopropylbenzenesulfonamide

In an analogous manner to Step 3, Example 317:

2-Isopropyl-5-(4-fluoro-benzenesulfonyl)-benzenesulfonyl chloride and 2-amino-1-pyridin-2-yl-ethanol were used to prepare 5-[(4-fluorophenyl)sulfonyl]-N-(2-hydroxy-2-pyridin-2-ylethyl)-2-isopropylbenzenesulfonamide.

MS (ES+) m/z 479.1;

HPLC purity Checked by AN LC/MS. MW confirmed.; Luna C8(2), 5 u, 4.6×250 mm column, 0.5 mL/min, Water/MeOH 20 min gradient.

HPLC purity 96.1% at 210-370 nm, 8.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₂H₂₃FN₂O₅S₂+H+, 479.11052; found (ESI, [M+H]⁺), 479.1099.

### EXAMPLE 728

### N-(2-hydroxy-2-pyridin-2-ylethyl)-2-isopropyl-5-(phenylsulfonyl)benzene-sulfonamide

In an analogous manner to Step 3, Example 295:

5-Benzenesulfonyl-2-isopropyl-benzenesulfonyl chloride and 2-amino-1-pyridin-2-yl-ethanol were used to prepare N-(2-hydroxy-2-pyridin-2-ylethyl)-2-isopropyl-5-(phenylsulfonyl)benzenesulfonamide.

MS (ES+) m/z 461.2;

HPLC purity Checked by AN LC/MS. MW confirmed by MS.; Luna C8(2), 5 u, 4.6×250 mm column, 0.5 mL/min, prep conditions.

HPLC purity 100% at 210-370 nm, 8.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₂H₂₄N₂O₅S₂+H+, 461.11994; found (ESI, [M+H]⁺), 461.118.

### EXAMPLE 729

### N-(2-hydroxy-2-pyridin-2-ylethyl)-2,4-dimethyl-5-(phenylsulfonyl)benzenesulfonamide

In an analogous manner to Step 3, Example 295:

5-Benzenesulfonyl-2,4-dimethyl-benzenesulfonyl chloride and 2-amino-1-pyridin-2-yl-ethanol were used to prepare N-(2-hydroxy-2-pyridin-2-ylethyl)-2,4-dimethyl-5-(phenylsulfonyl)benzenesulfonamide.

MS (ES+) m/z 447.1;

HPLC purity Checked by AN LC/MS. MW confirmed by MS.; Luna C8 (2), 5 u, 4.6×250 mm column, 0.5 mL/min, MeOH/water 20 min gradient.

HPLC purity 100% at 210-370 nm, 8.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₁H₂₂N₂O₅S₂+H+, 447.10429; found (ESI, [M+H]⁺), 447.1033.

### EXAMPLE 730

### 4-[({5-[(4-fluorophenyl)sulfonyl]-2-isopropylphenyl}sulfonyl)amino]-N-[4-(trifluoromethyl)phenyl]piperidine-1-carbothioamide

In an analogous manner to example 690, 5-[(4-fluorophenyl)sulfonyl]-2-isopropyl-N-piperidin-4-ylbenzenesulfonamide and 4-(trifluoromethyl)phenyl isothiocyanate were used to prepare 4-[({5-[(4-fluorophenyl)sulfonyl]-2-isopropylphenyl}sulfonyl)amino]-N-[4-(trifluoromethyl)phenyl]piperidine-1-carbothioamide.

MS (ES+) m/z 644.1;

HRMS: calcd. for C₂₈H₂₉F₄N₃O₄S₃+H⁺, 644.1335: found (ESI, [m+H]⁺), 644.1406.

### EXAMPLE 731

### 5-[(4-fluorophenyl)sulfonyl]-N-(2-hydroxy-2-pyridin-3-ylethyl)-2-isopropylbenzenesulfonamide

In an analogous manner to Step 3, Example 317:

2-Isopropyl-5-(4-fluoro-benzenesulfonyl)-benzenesulfonyl chloride and 2-amino-1-pyridin-3-yl-ethanol were used to prepare 5-[(4-fluorophenyl)sulfonyl]-N-(2-hydroxy-2-pyridin-3-ylethyl)-2-isopropylbenzenesulfonamide.

MS (ES+) m/z 479.1;

HPLC purity 96.7% at 210-370 nm, 8.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₂H₂₃FN₂O₅S₂+H+, 479.11052; found (ESI, [M+H]⁺), 479.1088.

### EXAMPLE 732

### 5-[(4-fluorophenyl)sulfonyl]-N-(2-hydroxy-2-phenylethyl)-2-isopropylbenzenesulfonamide

In an analogous manner to Step 3, Example 317:

2-Isopropyl-5-(4-fluoro-benzenesulfonyl)-benzenesulfonyl chloride and 2-amino-1-phenyl-ethanol were used to prepare 5-[(4-fluorophenyl)sulfonyl]-N-(2-hydroxy-2-phenylethyl)-2-isopropylbenzenesulfonamide.

MS (ES−) m/z 476.1;

HPLC purity 100% at 210-370 nm, 9.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₃H₂₄FNO₅S₂+NH₄⁺, 495.14182; found (ESI, [M+NH4]⁺), 495.1433.

### EXAMPLE 733

### 5-[(4-fluorophenyl)sulfonyl]-N-(2-hydroxy-1-methylethyl)-2-isopropylbenzenesulfonamide

In an analogous manner to Step 3, Example 317:

2-Isopropyl-5-(4-fluoro-benzenesulfonyl)-benzenesulfonyl chloride and 2-amino-1-propanol were used to prepare 5-[(4-fluorophenyl)sulfonyl]-N-(2-hydroxy-1-methylethyl)-2-isopropylbenzenesulfonamide.

MS (ES−) m/z 414.0;

HPLC purity 98.1% at 210-370 nm, 8.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₈H₂₂FNO₅S₂+H+, 416.09962; found (ESI, [M+H]⁺), 416.0975.

### EXAMPLE 734

### 5-[(4-fluorophenyl)sulfonyl]-N-[1-(hydroxymethyl)-2-methylpropyl]-2-isopropylbenzenesulfonamide

In an analogous manner to Step 3, Example 317:

2-Isopropyl-5-(4-fluoro-benzenesulfonyl)-benzenesulfonyl chloride and 2-amino-3-methyl-butan-1-ol were used to prepare 5-[(4-fluorophenyl)sulfonyl]-N-[1-(hydroxymethyl)-2-methylpropyl]-2-isopropylbenzenesulfonamide.

MS (ES+) m/z 444.1;

HPLC purity 96.4% at 210-370 nm, 9.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₀H₂₆FNO₅S₂+H+, 444.13092; found (ESI, [M+H]⁺), 444.1322.

### EXAMPLE 735

### N-(2-hydroxy-2-pyridin-3-ylethyl)-2-isopropyl-5-(phenylsulfonyl)benzenesulfonamide

In an analogous manner to Step 3, Example 295:

5-Benzenesulfonyl-2-isopropyl-benzenesulfonyl chloride and 2-amino-1-pyridin-3-yl-ethanol were used to prepare N-(2-hydroxy-2-pyridin-3-ylethyl)-2-isopropyl-5-(phenylsulfonyl)benzenesulfonamide.

MS (ES+) m/z 461.1;

HPLC purity 100% at 210-370 nm, 8.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₂H₂₄N₂O₅S₂+H+, 461.11994; found (ESI, [M+H]⁺), 461.1203.

### EXAMPLE 736

### N-(2-hydroxy-2-phenylethyl)-2-isopropyl-5-(phenylsulfonyl)benzenesulfonamide

In an analogous manner to Step 3, Example 295:

5-Benzenesulfonyl-2-isopropyl-benzenesulfonyl chloride and 2-amino-1-phenyl-ethanol were used to prepare N-(2-hydroxy-2-phenylethyl)-2-isopropyl-5-(phenylsulfonyl)benzenesulfonamide.

MS (ES−) m/z 458.1;

HPLC purity 97.2% at 210-370 nm, 9.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₃H₂₅NO₅S₂+NH₄⁺, 477.15124; found (ESI, [M+NH4]⁺), 477.1512.

### EXAMPLE 737

### N-(2-hydroxy-2-pyridin-3-ylethyl)-2,4-dimethyl-5-(phenylsulfonyl)benzenesulfonamide

In an analogous manner to Step 3, Example 295:

5-Benzenesulfonyl-2,4-dimethyl-benzenesulfonyl chloride and 2-amino-1-pyridin-3-yl-ethanol were used to prepare N-(2-hydroxy-2-pyridin-3-ylethyl)-2,4-dimethyl-5-(phenylsulfonyl)benzenesulfonamide.

MS (ES+) m/z 447.1;

HPLC purity 100% at 210-370 nm, 7.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₁H₂₂N₂O₅S₂+H+, 447.10429; found (ESI, [M+H]⁺), 447.1032.

### EXAMPLE 738

### 5-[(4-fluorophenyl)sulfonyl]-N-(2-hydroxybutyl)-2-isopropylbenzenesulfonamide

In an analogous manner to Step 3, Example 317:

2-Isopropyl-5-(4-fluoro-benzenesulfonyl)-benzenesulfonyl chloride and 1-aminobutan-2-ol were used to prepare 5-[(4-fluorophenyl)sulfonyl]-N-(2-hydroxybutyl)-2-isopropylbenzenesulfonamide.

MS (ES−) m/z 428.1;

HPLC purity 100% at 210-370 nm, 9.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₉H₂₄FNO₅S₂+H+, 430.11527; found (ESI, [M+H]⁺), 430.1147.

### EXAMPLE 739

### N-[2-(1H-Imidazol-1-yl)ethyl]-5-(phenylsulfonyl)-2-(trifluoromethyl)benzenesulfonamide

[2-(1H-Imidazol-1-yl)ethyl]amine dihydrochloride (97.3 mg, 0.529 mmol) was dissolved in 1 mL of water containing 169.3 mg (1.6 mmol) of sodium carbonate. Acetonitrile (4 mL) was then added. To this mixture 5-(phenylsulfonyl)-2-(trifluoromethyl)benzenesulfonyl chloride (201.8 mg, 0.524 mmol), prepared in Example 677, in 10 mL of acetonitrile was added dropwise over 1.5 h. After the addition the reaction was stirred at room temperature for 19 h (overnight). The reaction was filtered and the filtrate concentrated under reduced pressure to give 255.4 mg of a yellow oil. Purification of the oil on 50 g of silica gel (230-400 mesh) using 50% ethyl acetate-methylene chloride and then 5% methanol-methylene chloride as the eluents gave N-[2-(1H-imidazol-1-yl)ethyl]-5-(phenylsulfonyl)-2-(trifluoromethyl)benzenesulfonamide (78.4 mg, 33%) as a white solid.

MS (ES) m/z 460.0;

HRMS: calcd for C₁₈H₁₆F₃N₃O₄S₂+H⁺, 460.06071; found (ESI, [M+H]⁺), 460.0605;

Anal. Calcd for C₁₈H₁₆F₃N₃O₄S₂: C, 47.05; H, 3.51; N, 9.15. Found: C, 47.04; H, 3.14; N, 9.03.

### EXAMPLE 740

### 5-[(4-fluorophenyl)sulfonyl]-N-(2-hydroxy-2-pyridin-3-ylethyl)-2-methylbenzenesulfonamide

In an analogous manner to Step 3, Example 298:

2-Methyl-5-(4-fluoro-benzenesulfonyl)-benzenesulfonyl chloride and 2-amino-1-pyridin-3-yl-ethanol were used to prepare 5-[(4-fluorophenyl)sulfonyl]-N-(2-hydroxy-2-pyridin-3-ylethyl)-2-methylbenzenesulfonamide.

MS (ES+) m/z 451.1;

HPLC purity 100% at 210-370 nm, 7.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₀H₁₉FN₂O₅S₂+H+, 451.07922; found (ESI, [M+H]⁺), 451.0803.

### EXAMPLE 741

### N-(2-hydroxy-2-pyridin-3-ylethyl)-5-(phenylsulfonyl)-2-(trifluoromethyl)benzenesulfonamide

In an analogous manner to Step 3, Example 677:

5-Benzenesulfonyl-2-trifluoromethyl-benzenesulfonyl chloride and 2-amino-1-pyridin-3-yl-ethanol were used to prepare N-(2-hydroxy-2-pyridin-3-ylethyl)-5-(phenylsulfonyl)-2-(trifluoromethyl)benzenesulfonamide.

MS (ES+) m/z 487.1;

HPLC purity 98.0% at 210-370 nm, 7.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₀H₁₇F₃N₂O₅S₂+H+, 487.06037; found (ESI, [M+H]⁺), 487.0612.

### EXAMPLE 742

### tert-Butyl[2-({[5-(phenylsulfonyl)-2-(trifluoromethyl)phenyl]sulfonyl}amino)ethyl]carbamate

5-(Phenylsulfonyl)-2-(trifluoromethyl)benzenesulfonyl chloride (2.1471 g, 5.58 mmol), prepared in Example 677, in 25 mL of methylene chloride was added under nitrogen dropwise over 15 minutes to a solution of N-(2-aminoethyl)carbamic acid tert-butyl ester (883 [symbol] L, 5.58 mmol) and triethylamine (2.33 mL, 16.7 mmol) in 50 mL of methylene chloride at room temperature. After the addition the reaction was stirred at room temperature for 5.5 h. The reaction was extracted with 2 N HCl, dried (anhydrous magnesium sulfate) and the solvent removed under reduced pressure to give 2.71 g of a white solid. Purification of the solid on 300 g of silica gel (230-400 mesh) using 100% methylene chloride to 15% ethyl acetate-methylene chloride as the eluent gave tert-butyl[2-({[5-(phenylsulfonyl)-2-(trifluoromethyl)phenyl]sulfonyl}amino)ethyl]carbamate (2.05 g, 72%) as a white solid.

MS (ESI) m/z 507.

### EXAMPLE 743

### 5-[(3-methoxyphenyl)sulfonyl]-N-(tetrahydro-2H-pyran-4-yl)-2-(trifluoromethyl)benzenesulfonamide

Step 1: Following the same procedure described on example 677 (Step 2), 2-chloro-5-fluoronitrobenzene was used to prepare 2-nitro-4-fluorobenzotrifluoride.

Step 2: To a stirred solution of 2-nitro-4-fluorobenzotrifluoride (1.6 g, 7.65 mmol) in dimethylformamide (20 mL) was added 3-methoxybenzenethiol (1 mL, 8.0 mmol) and potassium carbonate (2.1 g, 15.3 mmol). The resulting mixture was stirred at room temperature 2 hours, diluted with water and extracted with ethyl acetate. The organic layer was washed several times with water, dried over magnesium sulfate and concentrated. The crude mixture was dissolved in methylene chloride (25 mL) and mCPBA (3.5 g 77%, 15.7 mmol) was added. The resulting mixture was stirred at room temperature for 30 minutes, washed with sodium dithionite solution and with sodium bicarbonate solution (sat). The organic layer was dried over magnesium sulfate and concentrated. Flash column separation using 0%-20% ethyl acetate/hexane gradient gave 5-(3-methoxyphenylsulfonyl)-2-trifluoromethyl-nitrobenzene (1.84 g, 65%).

Step 3: Following the same procedure described on example 677 (Step 3), 5-(3-methoxyphenylsulfonyl)-2-trifluoromethyl-nitrobenzene was used to prepare 5-(3-methoxyphenylsulfonyl)-2-trifluoromethylaniline.

Step 4: Following the same procedure described on example 677 (Step 4), 5-(3-methoxyphenylsulfonyl)-2-trifluoromethylaniline, was used to prepare 2-trifluoromethyl-5-(3-methoxylphenylsulfonyl)-benzenesulfonyl chloride.

Step 5: In an analogous manner to example 435, 2-trifluoromethyl-5-(3-methoxylphenylsulfonyl)-benzenesulfonyl chloride and tetrahydrofurfurylamine were used to prepare 5-[(3-methoxyphenyl)sulfonyl]-N-(tetrahydro-2H-pyran-4-yl)-2-(trifluoromethyl)benzenesulfonamide.

MS (ES−) m/z 478.0;

HPLC purity 97.2% at 210-370 nm, 9.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 744

### 5-[(3-hydroxyphenyl)sulfonyl]-N-(tetrahydro-2H-pyran-4-yl)-2-(trifluoromethyl)benzenesulfonamide

To a stirred solution of 5-[(3-methoxyphenyl)sulfonyl]-N-(tetrahydro-2H-pyran-4-yl)-2-(trifluoromethyl)benzenesulfonamide (0.08 g, 0.17 mmol) in methylene chloride (2 mL) was added cyclohexene (2 drops) and boron tribromide solution 1M in methylene chloride (0.6 mL, 0.6 mmol) at 0° C. The resulting solution was stirred 1 hour, quenched with methanol, and washed with sodium bicarbonate solution (sat). The organic layer was concentrated and the crude solid was triturated in methylene chloride to give 5-[(3-hydroxyphenyl)sulfonyl]-N-(tetrahydro-2H-pyran-4-yl)-2-(trifluoromethyl)benzenesulfonamide (0.04 g, 51%).

MS (ES−) m/z 464.0;

HPLC purity 95.7% at 210-370 nm, 8.5 min.; Xterra RP18, 3.5 u, 150×4.6 mm column, 1.2 mL/min, 85/15-5/95 (Ammon. Form. Buff. Ph=3.5/ACN+MeOH) for 1 min, hold 4 min.

HRMS: calcd for C₁₈H₁₈F₃NO₆S₂+H+, 466.06004; found (ESI, [M+H]⁺), 466.0625.

### EXAMPLE 745

### N-(2-cyanoethyl)-5-[(3-methoxyphenyl)sulfonyl]-2-(trifluoromethyl)benzenesulfonamide

In an analogous manner to example 435, 2-trifluoromethyl-5-(3-methoxylphenylsulfonyl)-benzenesulfonyl chloride and aminopropionitrile were used to prepare N-(2-cyanoethyl)-5-[(3-methoxyphenyl)sulfonyl]-2-(trifluoromethyl)benzenesulfonamide.

MS (ES−) m/z 447.0;

HPLC purity 97.9% at 210-370 nm, 8.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₇H₁₅F₃N₂O₅S₂+H+, 449.04472; found (ESI, [M+H]⁺), 449.0448.

### EXAMPLE 746

### N-[1-(4-tert-butylbenzoyl)piperidin-4-yl]-5-[(3-methoxyphenyl)sulfonyl]-2-(trifluoromethyl)benzenesulfonamide

Step 1: Following the same procedure described on example 462, piperidin-4-yl-carbamic acid tert-butyl ester and 4-tertbutylbenzoyl chloride were used to prepare [1-(4-tert-butyl-benzoyl)-piperidin-4-yl]-carbamic acid tert-butyl ester.

Step 2: Following the same procedure described on example 688, [1-(4-tert-butyl-benzoyl)-piperidin-4-yl]-carbamic acid tert-butyl ester was used to prepare (4-amino-piperidin-1-yl)-(4-tert-butyl-phenyl)-methanone.

Step 3: Following the same procedure described on example 435, 2-trifluoromethyl-5-(3-methoxylphenylsulfonyl)-benzenesulfonyl chloride and (4-amino-piperidin-1-yl)-(4-tert-butyl-phenyl)-methanone were used to prepare N-[1-(4-tert-butylbenzoyl)piperidin-4-yl]-5-[(3-methoxyphenyl)sulfonyl]-2-(trifluoromethyl)benzenesulfonamide.

MS (ES+) m/z 639.2;

HPLC purity 96.2% at 210-370 nm, 10.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₃₀H₃₃F₃N₂O₆S₂+H+, 639.18049; found (ESI, [M+H]⁺), 639.1804.

### EXAMPLE 747

### 5-[(3-methoxyphenyl)sulfonyl]-N-(2-pyridin-3-ylethyl)-2-(trifluoromethyl)benzenesulfonamide

In an analogous manner to example 435, 2-trifluoromethyl-5-(3-methoxylphenylsulfonyl)-benzenesulfonyl chloride and 3-(2-aminoethyl)pyridine were used to prepare 5-[(3-methoxyphenyl)sulfonyl]-N-(2-pyridin-3-ylethyl)-2-(trifluoromethyl)benzenesulfonamide.

MS (ES+) m/z 501.1;

HPLC purity 100% at 210-370 nm, 8.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₁H₁₉F₃N₂O₅S₂+H+, 501.07602; found (ESI, [M+H]⁺), 501.0756.

### EXAMPLE 748

### N-{1-[(4-tert-butylphenyl)sulfonyl]piperidin-4-yl}-5-(phenylsulfonyl)-2-(trifluoromethyl)benzenesulfonamide

In an analogous manner to example 462, 5-(phenylsulfonyl)-N-piperidin-4-yl-2-(trifluoromethyl)benzenesulfonamide and 4-tert-butylbenzenesulfonyl chloride were used to prepare N-{1-[(4-tert-butylphenyl)sulfonyl]piperidin-4-yl}-5-(phenylsulfonyl)-2-(trifluoromethyl)benzenesulfonamide.

MS (ES+) m/z 645.1;

HPLC purity 94.7% at 210-370 nm, 10.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₈H₃₁F₃N₂O₆S₃+H+, 645.13691; found (ESI, [M+H]⁺), 645.1376.

### EXAMPLE 749

### 5-(phenylsulfonyl)-2-(trifluoromethyl)-N-{1-[4-(trifluoromethyl)benzyl]piperidin-4-yl}benzenesulfonamide

In an analogous manner to example 462, 5-(phenylsulfonyl)-N-piperidin-4-yl-2-(trifluoromethyl)benzenesulfonamide and 4-trifluoromethylbenzyl bromide were used to prepare 5-(phenylsulfonyl)-2-(trifluoromethyl)-N-{1-[4-(trifluoromethyl)benzyl]piperidin-4-yl}benzenesulfonamide.

MS (ES+) m/z 607.1;

HPLC purity 96.8% at 210-370 nm, 9.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₆H₂₄F₆N₂O₄S₂+H+, 607.11544; found (ESI, [M+H]⁺), 607.1147.

### EXAMPLE 750

### N-[1-(cyanomethyl)piperidin-4-yl]-5-(phenylsulfonyl)-2-(trifluoromethyl)benzenesulfonamide

In an analogous manner to example 462, 5-(phenylsulfonyl)-N-piperidin-4-yl-2-(trifluoromethyl)benzenesulfonamide and bromoacetonitrile were used to prepare N-[1-(cyanomethyl)piperidin-4-yl]-5-(phenylsulfonyl)-2-(trifluoromethyl)benzenesulfonamide.

MS (ES+) m/z 488.1;

HPLC purity 96.7% at 210-370 nm, 8.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₀H₂₀F₃N₃O₄S₂+H+, 488.09201; found (ESI, [M+H]⁺), 488.0933.

### EXAMPLE 751

### N-[1-(2-oxo-2-phenylethyl)piperidin-4-yl]-5-(phenylsulfonyl)-2-(trifluoromethyl)benzenesulfonamide

In an analogous manner to example 462, 5-(phenylsulfonyl)-N-piperidin-4-yl-2-(trifluoromethyl)benzenesulfonamide and 2′-bromoacetophenone were used to prepare N-[1-(2-oxo-2-phenylethyl)piperidin-4-yl]-5-(phenylsulfonyl)-2-(trifluoromethyl)benzenesulfonamide.

MS (ES+) m/z 567.1;

HPLC purity 92.6% at 210-370 nm, 8.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₆H₂₅F₃N₂O₅S₂+H+, 567.12297; found (ESI, [M+H]⁺), 567.1232.

### EXAMPLE 752

### 5-[(3-chlorophenyl)sulfonyl]-N-(2-cyanoethyl)-2-(trifluoromethyl)benzenesulfonamide

In an analogous manner to example 743

Step 2: 3-chlorobenzenethiol and 2-nitro-4-fluorobenzotrifluoride were used to prepare 5-(3-chlorophenylsulfonyl)-2-trifluoromethyl-nitrobenzene.

Step 3: Following the same procedure described on example 677 (Step 3), 5-(3-chlorophenylsulfonyl)-2-trifluoromethyl-nitrobenzene was used to prepare 5-(3-chlorophenylsulfonyl)-2-trifluoromethylaniline.

Step 4: Following the same procedure described on example 677 (Step 4), 5-(3-chlorophenylsulfonyl)-2-trifluoromethylaniline, was used to prepare 2-trifluoromethyl-5-(3-chlorophenylsulfonyl)-benzenesulfonyl chloride.

Step 5: In an analogous manner to example 435, 2-trifluoromethyl-5-(3-chlorophenylsulfonyl)-benzenesulfonyl chloride and propionitrile were used to prepare 5-[(3-chlorophenyl)sulfonyl]-N-(2-cyanoethyl)-2-(trifluoromethyl)benzenesulfonamide.

MS (ES−) m/z 450.9;

HPLC purity 100% at 210-370 nm, 9.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 753

### N-[1-(4-tert-butylbenzoyl)piperidin-4-yl]-5-[(3-chlorophenyl)sulfonyl]-2-(trifluoromethyl)benzenesulfonamide

Step 1: Following the same procedure described on example 462, piperidin-4-yl-carbamic acid tert-butyl ester and 4-tertbutylbenzoyl chloride were used to prepare [1-(4-tert-butyl-benzoyl)-piperidin-4-yl]-carbamic acid tert-butyl ester.

Step 2: Following the same procedure described on example 688, [1-(4-tert-butyl-benzoyl)-piperidin-4-yl]-carbamic acid tert-butyl ester was used to prepare (4-amino-piperidin-1-yl)-(4-tert-butyl-phenyl)-methanone.

Step 3: Following the same procedure described on example 435, 2-trifluoromethyl-5-(3-chlorophenylsulfonyl)-benzenesulfonyl chloride and (4-amino-piperidin-1-yl)-(4-tert-butyl-phenyl)-methanone were used to prepare N-[1-(4-tert-butylbenzoyl)piperidin-4-yl]-5-[(3-chlorophenyl)sulfonyl]-2-(trifluoromethyl)benzenesulfonamide.

MS (ES+) m/z 643.2;

HPLC purity 99.3% at 210-370 nm, 11.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₉H₃₀ClF₃N₂O₅S₂+H+, 643.13095; found (ESI, [M+H]⁺), 643.1304.

### EXAMPLE 754

### 5-[(3-chlorophenyl)sulfonyl]-N-(tetrahydro-2H-pyran-4-yl)-2-(trifluoromethyl)benzenesulfonamide

In an analogous manner to example 435, 2-trifluoromethyl-5-(3-chlorophenylsulfonyl)-benzenesulfonyl chloride and tetrahydrofurfurylamine were used to prepare 5-[(3-chlorophenyl)sulfonyl]-N-(tetrahydro-2H-pyran-4-yl)-2-(trifluoromethyl)benzenesulfonamide.

MS (ES−) m/z 481.9;

HPLC purity 100% at 210-370 nm, 9.5 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 755

### 5-[(3-chlorophenyl)sulfonyl]-N-(2-pyridin-3-ylethyl)-2-(trifluoromethyl)benzenesulfonamide

In an analogous manner to example 435, 2-trifluoromethyl-5-(3-chlorophenylsulfonyl)-benzenesulfonyl chloride and 3-(2-aminoethyl)pyridine were used to prepare 5-[(3-chlorophenyl)sulfonyl]-N-(2-pyridin-3-ylethyl)-2-(trifluoromethyl)benzenesulfonamide.

MS (ES+) m/z 505.1;

HPLC purity 94.1% at 210-370 nm, 9.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₀H₁₆ClF₃N₂O₄S₂+H+, 505.02649; found (ESI, [M+H]⁺), 505.0247.

### EXAMPLE 756

### 5-[(4-fluorophenyl)sulfonyl]-N-(2-hydroxy-2-pyridin-2-ylethyl)-2-methylbenzenesulfonamide

In an analogous manner to Step 3, Example 298:

2-Methyl-5-(4-fluoro-benzenesulfonyl)-benzenesulfonyl chloride and 2-amino-1-pyridin-2-yl-ethanol were used to prepare 5-[(4-fluorophenyl)sulfonyl]-N-(2-hydroxy-2-pyridin-2-ylethyl)-2-methylbenzenesulfonamide.

MS (ES+) m/z 451.1;

HPLC purity 100% at 210-370 nm, 7.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₀H₁₉FN₂O₅S₂+H+, 451.07922; found (ESI, [M+H]⁺), 451.0801.

### EXAMPLE 757

### N-(2-hydroxy-2-pyridin-2-ylethyl)-5-(phenylsulfonyl)-2-(trifluoromethyl)benzenesulfonamide

In an analogous manner to Step 3, Example 677:

5-Benzenesulfonyl-2-trifluoromethyl-benzenesulfonyl chloride and 2-amino-1-pyridin-2-yl-ethanol were used to prepare N-(2-hydroxy-2-pyridin-2-ylethyl)-5-(phenylsulfonyl)-2-(trifluoromethyl)benzenesulfonamide.

MS (ES+) m/z 487.1;

HPLC purity 100% at 210-370 nm, 8.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₀H₁₇F₃N₂O₅S₂+H+, 487.06037; found (ESI, [M+H]⁺), 487.0581.

### EXAMPLE 758

### 5-(phenylsulfonyl)-N-{1-[2-(trifluoromethoxy)benzoyl]piperidin-4-yl}-2-(trifluoromethyl)benzenesulfonamide

In an analogous manner to example 462, 5-(phenylsulfonyl)-N-piperidin-4-yl-2-(trifluoromethyl)benzenesulfonamide and 2-trifluoromethoxybenzoyl chloride were used to prepare 5-(phenylsulfonyl)-N-{1-[2-(trifluoromethoxy)benzoyl]piperidin-4-yl}-2-(trifluoromethyl)benzenesulfonamide.

MS (ES+) m/z 637.1;

HPLC purity 96.0% at 210-370 nm, 9.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₆H₂₂F₆N₂O₆S₂+H+, 637.08962; found (ESI, [M+H]⁺), 637.0891.

### EXAMPLE 759

### N-(4-tert-butylphenyl)-4-({[5-(phenylsulfonyl)-2-(trifluoromethyl)phenyl]sulfonyl}amino)piperidine-1-carboxamide

In an analogous manner to example 462, 5-(phenylsulfonyl)-N-piperidin-4-yl-2-(trifluoromethyl)benzenesulfonamide and 4tertbutylphenylisocyanate were used to prepare N-(4-tert-butylphenyl)-4-({[5-(phenylsulfonyl)-2-(trifluoromethyl)phenyl]sulfonyl}amino)piperidine-1-carboxamide.

MS (ES+) m/z 624.2;

HPLC purity 97.0% at 210-370 nm, 10.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₉H₃₂F₃N₃O₅S₂+H+, 624.18082; found (ESI, [M+H]⁺), 624.1806.

### EXAMPLE 760

### N-(1-benzoylpiperidin-4-yl)-5-(phenylsulfonyl)-2-(trifluoromethyl)benzenesulfonamide

Step 1: In an analogous manner to example 462, piperidin-4-yl-carbamic acid tert-butyl ester and benzoyl chloride were used to prepare (1-benzoyl-piperidin-4-yl)-carbamic acid tert-butyl ester.

Step 2: In an analogous manner to example 680, (1-benzoyl-piperidin-4-yl)-carbamic acid tert-butyl ester was used to give 1-benzoyl-4-aminopiperidine.

Step 3: In an analogous manner to example 435, 2-trifluoromethyl-5-(phenylsulfonyl)-benzenesulfonyl chloride and 1-benzoyl-4-aminopiperidine were used to prepare N-(1-benzoylpiperidin-4-yl)-5-(phenylsulfonyl)-2-(trifluoromethyl)benzenesulfonamide.

MS (ES+) m/z 553.1;

HPLC purity 98.6% at 210-370 nm, 9.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₅H₂₃F₃N₂O₅S₂+H+, 553.10732; found (ESI, [M+H]⁺), 553.1075.

### EXAMPLE 761

### N-[1-(4-tert-butylbenzoyl)pyrrolidin-3-yl]-5-(phenylsulfonyl)-2-(trifluoromethyl)benzenesulfonamide

Step 1: In an analogous manner to example 462, 3-aminopyrrolidine-1-carboxylic acid tert-butyl ester and 4-tertbutylbenzoyl chloride were used to prepare [1-(4-tert-butyl-benzoyl)-pyrrolidin-3-yl]-carbamic acid tert-butyl ester.

Step 2: In an analogous manner to example 680, [1-(4-tert-butyl-benzoyl)-pyrrolidin-3-yl]-carbamic acid tert-butyl ester was used to give 1-(4-tertbutylbenzoyl)-3-aminopyrrolidine.

Step 3: In an analogous manner to example 435, 2-trifluoromethyl-5-(phenylsulfonyl)-benzenesulfonyl chloride and 1-(4-tertbutylbenzoyl)-3-aminopyrrolidine were used to prepare N-[1-(4-tert-butylbenzoyl)pyrrolidin-3-yl]-5-(phenylsulfonyl)-2-(trifluoromethyl)benzenesulfonamide.

MS (ES+) m/z 595.1;

HPLC purity 96.2% at 210-370 nm, 10.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₈H₂₉F₃N₂O₅S₂+H+, 595.15427; found (ESI, [M+H]⁺), 595.1547.

### EXAMPLE 762

### N-(2-Aminoethyl)-5-(phenylsulfonyl)-2-(trifluoromethyl)benzenesulfonamide hydrochloride

Approximately 100 mL of a saturated solution of anhydrous hydrogen chloride in ethyl acetate was added under nitrogen to a solution of tert-butyl[2-({[5-(phenylsulfonyl)-2-(trifluoromethyl)phenyl]sulfonyl}amino)ethyl]carbamate (2.00 g, 3.93 mmol), prepared in Example 742, in 100 mL of ethyl acetate at room temperature. After the addition the reaction was stirred for 5 h. The solid was collected by filtration, rinsed with ethyl acetate and dried under reduced pressure to give N-(2-aminoethyl)-5-(phenylsulfonyl)-2-(trifluoromethyl)benzenesulfonamide hydrochloride (1.6270 g, 93%) as a white solid.

MS (ES+) m/z 409.1;

HRMS: calcd for C₁₅H₁₅F₃N₂O₄S₂+H⁺, 409.04981; found (ESI, [M+H]⁺), 409.049.

### EXAMPLE 763

### 5-(phenylsulfonyl)-2-(trifluoromethyl)-N-{1-[4-(trifluoromethyl)phenyl]piperidin-4-yl}benzenesulfonamide

To a stirred solution of 5-(phenylsulfonyl)-N-piperidin-4-yl-2-(trifluoromethyl)benzenesulfonamide (0.09 g, 0.2 mmol) in dimethylformamide (1 mL) was added potassium carbonate (0.06 g, 0.43 mmol) and 4-fluorobenzotrifluoride (0.06 g, 0.37 mmol). The resulting solution was heated to 100° C. for three days and concentrated. The crude mixture was dissolved in methylene chloride, washed with ammonium chloride solution (sat.) and concentrated. Flash column separation using 0%-30% ethyl acetate/hexane gradient gave 5-(phenylsulfonyl)-2-(trifluoromethyl)-N-{1-[4-(trifluoromethyl)phenyl]piperidin-4-yl}benzenesulfonamide (0.032 g, 28%).

MS (ES+) m/z 593.1;

HPLC purity 98.1% at 210-370 nm, 11.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₅H₂₂F₆N₂O₄S₂+H+, 593.09979; found (ESI, [M+H]⁺), 593.1013.

### EXAMPLE 764

### N-[1-(4-tert-butylbenzoyl)piperidin-4-yl]-5-[(3-hydroxyphenyl)sulfonyl]-2-(trifluoromethyl)benzenesulfonamide

In an analogous manner to example 744, N-[1-(4-tert-butylbenzoyl)piperidin-4-yl]-5-[(3-methoxyphenyl)sulfonyl]-2-(trifluoromethyl)benzenesulfonamide was used to prepare N-[1-(4-tert-butylbenzoyl)piperidin-4-yl]-5-[(3-hydroxyphenyl)sulfonyl]-2-(trifluoromethyl)benzenesulfonamide.

MS (ES+) m/z 625.2;

HPLC purity 97.0% at 210-370 nm, 10.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₉H₃₁F₃N₂O₆S₂+H+, 625.16484; found (ESI, [M+H]⁺), 625.1625.

### EXAMPLE 765

### 5-(phenylsulfonyl)-N-(tetrahydro-2H-pyran-4-ylmethyl)-2-(trifluoromethyl)benzenesulfonamide

A stirred solution of 5-(phenylsulfonyl)-2-(trifluoromethyl)benzenesulfonyl chloride (0.101 g, 0.26 mmol) prepared in same matter as example 677, was taken up in dichloromethane (2 ml). 1.2 eq of 4-Aminomethyltetrahydropyran and 1.5 eq. trietylamine was syringed into the reaction vial and was allowed to stir overnight at room temperature. The product was transferred onto a 4 g Isco RediSep® Normal Phase column and was purified by automated flash chromatography using a gradient of 20% to 100% hexane/ethyl acetate. Isolation of the main component gave the title compound of 5-(phenylsulfonyl)-N-(tetrahydro-2H-pyran-4-ylmethyl)-2-(trifluoromethyl)benzenesulfonamide (29 mg, 23.8%) as a white solid.

MS (ES+) m/z 464.0;

HPLC purity 98.9% at 210-370 nm, 8.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₉H₂₀F₃NO₅S₂+H+, 464.08077; found (ESI, [M+H]+), 464.079.

### EXAMPLE 766

### N-(2-morpholin-4-ylethyl)-5-(phenylsulfonyl)-2-(trifluoromethyl)benzenesulfonamide

In an analogous manner to example 765, 5-(phenylsulfonyl)-2-(trifluoromethyl)benzenesulfonyl chloride and 2-morpholinoethanamine was used to prepare the title compound of N-(2-morpholin-4-ylethyl)-5-(phenylsulfonyl)-2-(trifluoromethyl)benzenesulfonamide (99 mg, 78.8%) as a white solid.

MS (ES+) m/z 479.1;

HPLC purity 100% at 210-370 nm, 7.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₉H₂₁F₃N₂O₅S₂+H+, 479.09167; found (ESI, [M+H]+), 479.0921.

### EXAMPLE 767

### N-(3-morpholin-4-ylpropyl)-5-(phenylsulfonyl)-2-(trifluoromethyl)benzenesulfonamide

In an analogous manner to example 765, 5-(phenylsulfonyl)-2-(trifluoromethyl)benzenesulfonyl chloride and N-(3-Aminopropyl)-morpholine was used to prepare the title compound N-(3-morpholin-4-ylpropyl)-5-(phenylsulfonyl)-2-(trifluoromethyl)benzenesulfonamide (72 mg, 55.7%) as a white solid.

MS (ES+) m/z 493.1;

HPLC purity 93.8% at 210-370 nm, 6.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₀H₂₃F₃N₂O₅S₂+H+, 493.10732; found (ESI, [M+H]+), 493.1053;

### EXAMPLE 768

### N-(3-methoxypropyl)-5-(phenylsulfonyl)-2-(trifluoromethyl)benzenesulfonamide

In an analogous manner to example 765, 5-(phenylsulfonyl)-2-(trifluoromethyl)benzenesulfonyl chloride and 3-methoxypropylamine was used to prepare the title compound N-(3-methoxypropyl)-5-(phenylsulfonyl)-2-(trifluoromethyl)benzenesulfonamide (75 mg, 65.3%) as a white solid.

MS (ES+) m/z 438.1;

HPLC purity 100% at 210-370 nm, 8.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₇H₁₈F₃NO₅S₂+H+, 438.06512; found (ESI, [M+H]+), 438.064.

### EXAMPLE 769

### N-[1-(hydroxymethyl)-2-methylpropyl]-5-(phenylsulfonyl)-2-(trifluoromethyl)benzenesulfonamide

In an analogous manner to example 765, 5-(phenylsulfonyl)-2-(trifluoromethyl)benzenesulfonyl chloride and 2-amino-3-methylbutanol was used to prepare the title compound N-[1-(hydroxymethyl)-2-methylpropyl]-5-(phenylsulfonyl)-2-(trifluoromethyl)benzenesulfonamide (27.5 mg, 23%) as a white solid.

MS (ES+) m/z 452;

HPLC purity 89.5% at 210-370 nm, 8.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₈H₂₀F₃NO₅S₂+H+, 452.08077; found (ESI, [M+H]+), 452.0813.

### EXAMPLE 770

### N-[2-({[5-(Phenylsulfonyl)-2-(trifluoromethyl)phenyl]sulfonyl}amino)ethyl]benzamide

Benzoyl chloride (39 μL, 0.336 mmol) was added under nitrogen to a mixture of N-(2-aminoethyl)-5-(phenylsulfonyl)-2-(trifluoromethyl)benzenesulfonamide hydrochloride (150.7 mg, 0.339 mmole), prepared in Example 762, and triethylamine (57 μL, 0.409 mmol) in 20 mL of methylene chloride at room temperature. After the addition the reaction was stirred at room temperature for approximately 24 h. An additional 57 μL (0.409 mmol) of triethylamine and 20 μL (0.172 mmol) of benzoyl chloride were added and the reaction was stirred for 4 h at room temperature. The reaction was extracted with 2 N HCl. The organic layer was separated and the aqueous layer was extracted three times with 10% methanol-methylene chloride. The combined organic extracts were dried (anhydrous magnesium sulfate) and the solvent removed under reduced pressure to give 183 mg of a solid. Putification of the solid on a 12 g Redi Sep Flash Column (silica gel) using a gradient of 100% methylene chloride to 20% ethyl acetate-methylene chloride as the eluent gave N-[2-({[5-(phenylsulfonyl)-2-(trifluoromethyl)phenyl]sulfonyl}amino)ethyl]benzamide (106.2 mg, 61%) as a white solid.

MS (ESI+) m/z 513;

HRMS: calcd for C₂₂H₁₉F₃N₂O₅S₂+H⁺, 513.07602; found (ESI, [M+H]⁺), 513.0763;

Anal. Calcd for C₂₂H₁₉F₃N₂O₅S₂: C, 51.56; H, 3.74; N, 5.47. Found: C, 51.53; H, 3.58; N, 5.40.

### EXAMPLE 771

### 5-(phenylsulfonyl)-N-(2-pyridin-4-ylethyl)-2-(trifluoromethyl)benzenesulfonamide

In an analogous manner to example 765, 5-(phenylsulfonyl)-2-(trifluoromethyl)benzenesulfonyl chloride and 2-(pyridine-4-yl)ethanamine was used to prepare the title compound 5-(phenylsulfonyl)-N-(2-pyridin-4-ylethyl)-2-(trifluoromethyl)benzenesulfonamide (40.4 mg, 38%) as a white solid.

MS (ES+) m/z 471.1;

HPLC purity 100% at 210-370 nm, 8.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₀H₁₇F₃N₂O₄S₂+H+, 471.06546; found (ESI, [M+H]+), 471.0663.

### EXAMPLE 772

### N-(2,3-dihydro-1H-inden-2-yl)-5-(phenylsulfonyl)-2-(trifluoromethyl)benzenesulfonamide

In an analogous manner to example 765, 5-(phenylsulfonyl)-2-(trifluoromethyl)benzenesulfonyl chloride and 2-aminoindan was used to prepare the title compound N-(2,3-dihydro-1H-inden-2-yl)-5-(phenylsulfonyl)-2-(trifluoromethyl)benzenesulfonamide (56.2 mg, 44%) as a white solid.

MS (ES−) m/z 480.0;

HPLC purity 91.5% at 210-370 nm, 10.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 773

### N-[(2R)-2-hydroxy-2-phenylethyl]-5-(phenylsulfonyl)-2-(trifluoromethyl)benzenesulfonamide

In an analogous manner to example 765, 5-(phenylsulfonyl)-2-(trifluoromethyl)benzenesulfonyl chloride and (R)-2-amino-1-phenylethanol was used to prepare the title compound N-[(2R)-2-hydroxy-2-phenylethyl]-5-(phenylsulfonyl)-2-(trifluoromethyl)benzenesulfonamide (24.6 mg, 19%) as a white solid.

MS (ES−) m/z 484.0;

HPLC purity 100% at 210-370 nm, 9.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 774

### N-[(2S)-2-hydroxy-2-phenylethyl]-5-(phenylsulfonyl)-2-(trifluoromethyl)benzenesulfonamide

In an analogous manner to example 765, 5-(phenylsulfonyl)-2-(trifluoromethyl)benzenesulfonyl chloride and (S)-2-amino-1-phenylethanol was used to prepare the title compound N-[(2S)-2-hydroxy-2-phenylethyl]-5-(phenylsulfonyl)-2-(trifluoromethyl)benzenesulfonamide (45.5 mg, 36%) as a white solid.

MS (ES−) m/z 484.0;

HPLC purity 100% at 210-370 nm, 9.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 775

### 4-Methyl-N-[2-({[5-(phenylsulfonyl)-2-(trifluoromethyl)phenyl]sulfonyl}amino)ethyl]benzamide

In an analogous manner as described in Example 770, replacing benzoyl chloride with 4-methyl-benzoyl chloride and using a gradient of 100% methylene chloride to 50% ethyl acetate-methylene chloride as the eluent gave 4-methyl-N-[2-({[5-(phenylsulfonyl)-2-(trifluoromethyl)phenyl]sulfonyl}amino)ethyl]benzamide (105.1 mg, 59%) as a white solid.

MS (ES+) m/z 527.1;

HRMS: calcd for C₂₃H₂₁F₃N₂O₅S₂+H⁺, 527.09167; found (ESI, [M+H]⁺), 527.0905;

Anal. Calcd for C₂₃H₂₁F₃N₂O₅S₂: C, 52.46; H, 4.02; N, 5.32. Found: C, 52.28; H, 4.05; N, 5.12.

### EXAMPLE 776

### 4-tert-Butyl-N-[2-({[5-(phenylsulfonyl)-2-(trifluoromethyl)phenyl]sulfonyl}amino)ethyl]benzamide

In an analogous manner as described in Example 775, and replacing 4-methyl-benzoyl chloride with 4-tert-butyl-benzoyl chloride, gave 4-tert-butyl-N-[2-({[5-(phenylsulfonyl)-2-(trifluoromethyl)phenyl]sulfonyl}amino)ethyl]benzamide (134.2 mg, 70%) as a white solid.

MS (ES+) m/z 569.2;

HRMS: calcd for C₂₆H₂₇F₃N₂O₅S₂+H⁺, 569.13862; found (ESI, [M+H]⁺), 569.1392;

Anal. Calcd for C₂₆H₂₇F₃N₂O₅S₂: C, 54.92; H, 4.79; N, 4.93. Found: C, 54.89; H, 4.74; N, 4.77.

### EXAMPLE 777

### 4-Fluoro-N-[2-({[5-(phenylsulfonyl)-2-(trifluoromethyl)phenyl]sulfonyl}amino)ethyl]benzamide

In an analogous manner as described in Example 775, and replacing 4-methyl-benzoyl chloride with 4-fluoro-benzoyl chloride, gave 4-fluoro-N-[2-({[5-(phenylsulfonyl)-2-(trifluoromethyl)phenyl]sulfonyl}amino)ethyl]benzamide (96.8 mg, 54%) as a white solid.

MS (ES+) m/z 531.0;

HRMS: calcd for C₂₂H₁₈F₄N₂O₅S₂+H⁺, 531.06660; found (ESI, [M+H]⁺), 531.0664;

Anal. Calcd for C₂₂H₁₈F₄N₂O₅S₂: C, 49.81; H, 3.42; N, 5.28. Found: C, 49.71; H, 3.42; N, 5.09.

### EXAMPLE 778

### 4-Chloro-N-[2-({[5-(phenylsulfonyl)-2-(trifluoromethyl)phenyl]sulfonyl}amino)ethyl]benzamide

In an analogous manner as described in Example 775, and replacing 4-methyl-benzoyl chloride with 4-chloro-benzoyl chloride, gave 4-chloro-N-[2-({[5-(phenylsulfonyl)-2-(trifluoromethyl)phenyl]sulfonyl}amino)ethyl]benzamide (131.2 mg, 71%) as a white solid.

MS (ES+) m/z 547.0;

HRMS: calcd for C₂₂H₁₈ClF₃N₂O₅S₂+H⁺, 547.03705; found (ESI, [M+H]⁺), 547.0378;

Anal. Calcd for C₂₂H₁₈ClF₃N₂O₅S₂: C, 48.31; H, 3.32; N, 5.12. Found: C, 48.15; H, 3.38; N, 4.95.

### EXAMPLE 779

### 4-Bromo-N-[2-({[5-(phenylsulfonyl)-2-(trifluoromethyl)phenyl]sulfonyl}amino)ethyl]benzamide

In an analogous manner as described in Example 775, and replacing 4-methyl-benzoyl chloride with 4-bromo-benzoyl chloride, gave 4-bromo-N-[2-({[5-(phenylsulfonyl)-2-(trifluoromethyl)phenyl]sulfonyl}amino)ethyl]benzamide (149.3 mg, 74%) as a white solid.

MS (ESI+) m/z 591;

HRMS: calcd for C₂₂H₁₈BrF₃N₂O₅S₂+H⁺, 590.98653; found (ESI, [M+H]⁺), 590.9851;

Anal. Calcd for C₂₂H₁₈BrF₃N₂O₅S₂: C, 44.68; H, 3.07; N, 4.74. Found: C, 44.57; H, 3.32; N, 4.55.

### EXAMPLE 780

### 4-Methoxy-N-[2-({[5-(phenylsulfonyl)-2-(trifluoromethyl)phenyl]sulfonyl}amino)ethyl]benzamide

In an analogous manner as described in Example 775, and replacing 4-methyl-benzoyl chloride with 4-methoxy-benzoyl chloride, gave 4-methoxy-N-[2-({[5-(phenylsulfonyl)-2-(trifluoromethyl)phenyl]sulfonyl}amino)ethyl]benzamide (163.5 mg, 89%) as a white solid.

MS (ES+) m/z 543.1;

HRMS: calcd for C₂₃H₂₁F₃N₂O₆S₂+H⁺, 543.08659; found (ESI, [M+H]⁺), 543.0869;

Anal. Calcd for C₂₃H₂₁F₃N₂O₆S₂: C, 50.92; H, 3.90; N, 5.16. Found: C, 51.11; H, 3.71; N, 4.94.

### EXAMPLE 781

### N-[2-({[5-(Phenylsulfonyl)-2-(trifluoromethyl)phenyl]sulfonyl}amino)ethyl]-4-(trifluoromethyl)benzamide

In an analogous manner as described in Example 775, and replacing 4-methyl-benzoyl chloride with 4-trifluoromethyl-benzoyl chloride, gave N-[2-({[5-(phenylsulfonyl)-2-(trifluoromethyl)phenyl]sulfonyl}amino)ethyl]-4-(trifluoromethyl)benzamide (118.8 mg, 60%) as a white solid.

MS (ES+) m/z 581.0;

HRMS: calcd for C₂₃H₁₈F₆N₂O₅S₂+H⁺, 581.06341; found (ESI, [M+H]⁺), 581.0607;

Anal. Calcd for C₂₃H₁₈F₆N₂O₅S₂: C, 47.59; H, 3.13; N, 4.83. Found: C, 47.45; H, 2.84; N, 4.63.

### EXAMPLE 782

### N-[2-({[5-(Phenylsulfonyl)-2-(trifluoromethyl)phenyl]sulfonyl}amino)ethyl]-4-(trifluoromethoxy)benzamide

In an analogous manner as described in Example 775, and replacing 4-methyl-benzoyl chloride with 4-trifluoromethoxy-benzoyl chloride, gave N-[2-({[5-(phenylsulfonyl)-2-(trifluoromethyl)phenyl]sulfonyl}amino)ethyl]-4-(trifluoromethoxy)benzamide (122.8 mg, 60%) as a white solid.

MS (ES+) m/z 597.0;

HRMS: calcd for C₂₃H₁₈F₆N₂O₆S₂+H⁺, 597.05832; found (ESI, [M+H]⁺), 597.0574;

Anal. Calcd for C₂₃H₁₈F₆N₂O₆S₂: C, 46.31; H, 3.04; N, 4.70. Found: C, 46.24; H, 2.59; N, 4.49.

### EXAMPLE 783

### N-[2-({[5-(Phenylsulfonyl)-2-(trifluoromethyl)phenyl]sulfonyl}amino)ethyl]isonicotinamide

Isonicotinoyl chloride (61.5 mg, 0.345 mmol) was added under nitrogen to a mixture of N-(2-aminoethyl)-5-(phenylsulfonyl)-2-(trifluoromethyl)benzenesulfonamide hydrochloride (150.1 mg, 0.337 mmol), prepared in Example 762, and triethylamine (141 [symbol] L, 1.01 mmol) in 20 mL of methylene chloride at room temperature. After the addition the reaction was stirred at room temperature for approximately 24 h. The reaction was partitioned with water. The organic layer was separated and the aqueous layer was extracted three times with methylene chloride. The combined extracts were dried (anhydrous magnesium sulfate) and the solvent removed under reduced pressure to give 186.8 mg of a solid. Purification of the solid on a 12 g Redi Sep Flash Column (silica gel) using a gradient of 100% methylene chloride to 20% methanol-methylene chloride as the eluent gave N-[2-({[5-(phenylsulfonyl)-2-(trifluoromethyl)phenyl]sulfonyl}amino)ethyl]isonicotinamide (153.5 mg, 89%) as a white solid.

MS (ES+) m/z 514.1;

HRMS: calcd for C₂₁H₁₈F₃N₃O₅S₂+H⁺, 514.07127; found (ESI, [M+H]⁺), 514.0713;

Anal. Calcd for C₂₁H₁₈F₃N₃O₅S₂: C, 49.12; H, 3.53; N, 8.18. Found: C, 48.98; H, 2.94; N, 8.04.

### EXAMPLE 784

### N-[(1R)-1-(hydroxymethyl)propyl]-5-(phenylsulfonyl)-2-(trifluoromethyl)benzenesulfonamide

In an analogous manner to example 765, 5-(phenylsulfonyl)-2-(trifluoromethyl)benzenesulfonyl chloride and (S)-(+)-2-amino-1-butanol was used to prepare the title compound N-[(1R)-1-(hydroxymethyl)propyl]-5-(phenylsulfonyl)-2-(trifluoromethyl)benzenesulfonamide (74.6 mg, 65%) as a white solid.

MS (ES+) m/z 438.0;

HPLC purity 100% at 210-370 nm, 8.5 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₇H₁₈F₃NO₅S₂+H+, 438.06512; found (ESI, [M+H]+), 438.0621.

### EXAMPLE 785

### N-(2-hydroxyethyl)-5-[(3-methoxyphenyl)sulfonyl]-2-(trifluoromethyl)benzenesulfonamide

In an analogous manner to example 654, 2-trifluoromethyl-5-(3-methoxylphenylsulfonyl)-benzenesulfonyl chloride and ethanolamine were used to prepare N-(2-hydroxyethyl)-5-[(3-methoxyphenyl)sulfonyl]-2-(trifluoromethyl)benzenesulfonamide.

MS (ES−) m/z 438.0;

HPLC purity 100% at 210-370 nm, 8.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₆H₁₆F₃NO₆S₂+H+, 440.04439; found (ESI, [M+H]⁺), 440.0446.

### EXAMPLE 786

### tert-butyl(3S)-3-({[5-(phenylsulfonyl)-2-(trifluoromethyl)phenyl]sulfonyl}amino)piperidine-1-carboxylate

In an analogous manner to example 435, 2-trifluoromethyl-5-(phenylsulfonyl)-benzenesulfonyl chloride and S-3-aminopiperidine-1-carboxylic acid tert-butyl ester were used to prepare tert-butyl(3S)-3-({[5-(phenylsulfonyl)-2-(trifluoromethyl)phenyl]sulfonyl}amino)piperidine-1-carboxylate.

MS (ES−) m/z 547.0;

HPLC purity 96.6% at 210-370 nm, 10.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₃H₂₇F₃N₂O₆S₂+H+, 549.13354; found (ESI, [M+H]⁺), 549.1335.

### EXAMPLE 787

### tert-butyl(3R)-3-({[5-(phenylsulfonyl)-2-(trifluoromethyl)phenyl]sulfonyl}amino)piperidine-1-carboxylate

In an analogous manner to example 435, 2-trifluoromethyl-5-(phenylsulfonyl)-benzenesulfonyl chloride and R-3-aminopiperidine-1-carboxylic acid tert-butyl ester were used to prepare tert-butyl(3R)-3-({[5-(phenylsulfonyl)-2-(trifluoromethyl)phenyl]sulfonyl}amino)piperidine-1-carboxylate.

MS (ES−) m/z 547.0;

HPLC purity 97.3% at 210-370 nm, 10.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₃H₂₇F₃N₂O₆S₂+H+, 549.13354; found (ESI, [M+H]⁺), 549.1335.

### EXAMPLE 788

### N-[1-(2-hydroxyethyl)piperidin-4-yl]-5-(phenylsulfonyl)-2-(trifluoromethyl)benzenesulfonamide

To a stirred solution of 5-(phenylsulfonyl)-N-piperidin-4-yl-2-(trifluoromethyl)benzenesulfonamide (0.09 g, 0.20 mmol) in 1,4-dioxane (1 mL) was added 2-bromoethanol (0.05, 0.40 mmol) and triethylamine (0.1 mL, 0.7 mmol). The resulting solution was heated 150° C. in a microwave for 10 minutes and concentrated. Flash column separation using 0%-5% methanol/methylene chloride gradient gave N-[1-(2-hydroxyethyl)piperidin-4-yl]-5-(phenylsulfonyl)-2-(trifluoromethyl)benzenesulfonamide. (0.03 g, 30%).

MS (ES+) m/z 493.1;

HPLC purity 98.3% at 210-370 nm, 6.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₀H₂₃F₃N₂O₅S₂+H+, 493.10732; found (ESI, [M+H]⁺), 493.1083.

### EXAMPLE 789

### tert-butyl 4-[({5-[(4-fluorophenyl)sulfonyl]-2-methylphenyl}sulfonyl)amino]piperidine-1-carboxylate

To a solution of 5-(4-fluoro-benzenesulfonyl)-2-methyl-benzenesulfonyl chloride (1.0 g, 2.87 mmol) in dichloromethane (20.0 mL) was added triethylamine (0.80 mL, 5.73 mmol) followed by 4-amino-1-Boc-piperidne (0.69 g, 3.44 mmol). The reaction was stirred overnight at room temperature under nitrogen. The following day the reaction was concentrated down onto silica gel and purified using automated chromatography with a gradient mobile phase consisting of ethyl acetate and hexane resulting in the isolation of tert-butyl 4-[({5-[(4-fluorophenyl)sulfonyl]-2-methylphenyl}sulfonyl)amino]piperidine-1-carboxylate (1.42 g, 97%).

MS (ES−) m/z 511.0.

### EXAMPLE 790

### 5-(phenylsulfonyl)-N-[(3S)-piperidin-3-yl]-2-(trifluoromethyl)benzenesulfonamide

In an analogous manner to example 688, tert-butyl(3S)-3-({[5-(phenylsulfonyl)-2-(trifluoromethyl)phenyl]sulfonyl}amino)piperidine-1-carboxylate was used to prepare 5-(phenylsulfonyl)-N-[(3S)-piperidin-3-yl]-2-(trifluoromethyl)benzenesulfonamide.

MS (ES+) m/z 449.1;

HPLC purity 97.1% at 210-370 nm, 6.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₈H₁₉F₃N₂O₄S₂+H+, 449.08111; found (ESI, [+H]⁺), 449.0825.

### EXAMPLE 791

### 5-(phenylsulfonyl)-N-[(3R)-piperidin-3-yl]-2-(trifluoromethyl)benzenesulfonamide

In an analogous manner to example 688, tert-butyl(3R)-3-({[5-(phenylsulfonyl)-2-(trifluoromethyl)phenyl]sulfonyl}amino)piperidine-1-carboxylate was used to prepare 5-(phenylsulfonyl)-N-[(3R)-piperidin-3-yl]-2-(trifluoromethyl)benzenesulfonamide.

MS (ES+) m/z 449.1;

HPLC purity 96.9% at 210-370 nm, 6.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₈H₁₉F₃N₂O₄S₂+H+, 449.08111; found (ESI, [M+H]⁺), 449.0822.

### EXAMPLE 792

### 5-[(1,2-dimethyl-1H-indol-5-yl)sulfonyl]-N-piperidin-4-yl-2-(trifluoromethyl)benzenesulfonamide

Step 1: To a stirred solution of 4-fluoro-2-bromobenzotrifluoride (2.0 g, 8.23 mmol) in THF (50 mL) at −78° C. was added nButyl lithium 2.5M in hexane (4.0 mL, 10.0 mmol) dropwise over 5 minutes. The resulting solution was stirred for 15 minutes, then sulfur dioxide was bubbled in over 20 minutes, and the solution as concentrated. The crude mixture was taken up in methylene chloride (50 mL) and N-Chlorosuccinimide (1.1 g, 8.23 mmol) was added. The resulting solution was stirred room temperature for 1.5 hrs, washed with ammonium chloride solution (sat.) and concentrated. Flash column separation using 0%-30% ethyl acetate/hexane gradient gave 5-fluoro-2-trifluoromethylbenzenesulfonyl chloride (0.55 g, 25%).

Step 2: In an analogous manner to example 435, 5-fluoro-2-trifluoromethylbenzenesulfonyl chloride and 4-aminopiperidine-1-carboxylic acid tert-butyl ester were used to prepare tert-butyl 4-({[5-fluoro-2-(trifluoromethyl)phenyl]sulfonyl}amino)piperidine-1-carboxylate.

Step 3: To a stirred solution of 5-bromo-2-methyl-indole (5.0 g, 23.5 mmol) in DMF (50 mL) at 0° C. was added sodium hydride (1.13 g (60%), 28.05 mmol) and the resulting solution was stirred for 5 minutes. Methyl iodide (3.3 mL, 52.8 mmol) was added and the reaction was allowed to warm to room temperature. After 1 hr, the reaction was quenched with ammonium chloride solution (sat). and extracted several times with ethyl acetate. The combined organic layers were washed with brine, dried over magnesium sulfate, and concentrated. Flash column separation using 0%-10% ethyl acetate/hexane gradient gave crude 1,2-dimethyl-5-bromoindole. This material was dissolved in TIF (30 mL) and chilled to −78° C. nButyl lithium 2.5M in hexane (4.0 mL, 10.0 mmol) was added dropwise. After 10 minutes sulfur dioxide was bubbled into the reaction mixture for 15 minutes. The mixture was allowed to warm to room temperature and concentrated to give 1,2-dimethylindole-5-sulfinic acid lithium salt.

HRMS: calcd for C₂₁H₂₅F₃N₂O₆S₂+H+, 523.11789; found (ESI, [M+H—C₄H₈]⁺), 467.0601. C₄H₈.

Step 4: To a stirred solution of 1,2-dimethylindole-5-sulfinic acid lithium salt (0.10 g, 0.54 mmol) in dimethylformamide (1 mL), tert-butyl 4-({[5-fluoro-2-(trifluoromethyl)phenyl]sulfonyl}amino)piperidine-1-carboxylate (0.115 g, 0.27 mmol) was added and the resulting solution was heated to 80° C. overnight and concentrated. The crude material was taken up in ethyl acetate, washed with sodium bicarbonate solution (sat) and concentrated. Flash column separation using 0%-40% ethyl acetate/hexane gradient gave tert-butyl 4-({[5-(1,2-dimethyl-1H-indol-5-yl)sulfonyl)-2-(trifluoromethyl)phenyl]sulfonyl}amino)piperidine-1-carboxylate.

Step 5: In an analogous manner to example 688, tert-butyl 4-({[5-(1,2-dimethyl-1H-indol-5-yl)sulfonyl)-2-(trifluoromethyl)phenyl]sulfonyl}amino)piperidine-1-carboxylate was used to prepare 5-[(1,2-dimethyl-1H-indol-5-yl)sulfonyl]-N-piperidin-4-yl-2-(trifluoromethyl)benzenesulfonamide.

MS (ES+) m/z 516.1;

HPLC purity 100% at 210-370 nm, 8.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₂H₂₄F₃N₃O₄S₂+H+, 516.12331; found (ESI, [M+H]⁺), 516.1237.

### EXAMPLE 793

### tert-Butyl methyl[2-({[5-(phenylsulfonyl)-2-(trifluoromethyl)phenyl]sulfonyl}amino)ethyl]carbamate

5-(Phenylsulfonyl)-2-(trifluoromethyl)benzenesulfonyl chloride (2.6940 g, 7.00 mmol), prepared in Example 677, in 25 mL of methylene chloride was added under nitrogen dropwise over 30 minutes to a solution of N-(2-aminoethyl)-N-methyl carbamic acid tert-butyl ester (1.25 mL, 6.99 mmol) and triethylamine (2.93 mL, 21.0 mmol) in 50 mL of methylene chloride at room temperature. After the addition the reaction was stirred at room temperature for 7 h. The reaction was extracted with 2 N HCl. The organic layer was separated and the aqueous layer was extracted three times with methylene chloride. The combined extracts were dried (anhydrous magnesium sulfate) and the solvent removed under reduced pressure to give 2.98 g of a yellow foam. Purification of the foam on 500 g of silica gel (230-400 mesh) using 10% ethyl acetate-methylene chloride to 20% ethyl acetate-methylene chloride as the eluent gave tert-butyl methyl[2-({[5-(phenylsulfonyl)-2-(trifluoromethyl)phenyl]sulfonyl}amino)ethyl]carbamate (862.1 mg, 24%) as a yellow foam.

MS (ES) m/z 521.0.

### EXAMPLE 794

### tert-butyl(3S)-3-({[5-(phenylsulfonyl)-2-(trifluoromethyl)phenyl]sulfonyl}amino)pyrrolidine-1-carboxylate

In an analogous manner to example 435, 2-trifluoromethyl-5-(phenylsulfonyl)-benzenesulfonyl chloride and S-3-aminopyrrolidine-1-carboxylic acid tert-butyl ester were used to prepare tert-butyl(3S)-3-({[5-(phenylsulfonyl)-2-(trifluoromethyl)phenyl]sulfonyl}amino)pyrrolidine-1-carboxylate.

MS (ES−) m/z 533.0;

HPLC purity 97.3% at 210-370 nm, 9.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 795

### tert-butyl(3R)-3-({[5-(phenylsulfonyl)-2-(trifluoromethyl)phenyl]sulfonyl}amino)pyrrolidine-1-carboxylate

In an analogous manner to example 435, 2-trifluoromethyl-5-(phenylsulfonyl)-benzenesulfonyl chloride and R-3-aminopyrrolidine-1-carboxylic acid tert-butyl ester were used to prepare tert-butyl(3R)-3-({[5-(phenylsulfonyl)-2-(trifluoromethyl)phenyl]sulfonyl}amino)pyrrolidine-1-carboxylate.

HPLC purity 97.6% at 210-370 nm, 9.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

MS (ES−) m/z 533.0.

### EXAMPLE 796

### 5-(phenylsulfonyl)-N-[(3S)-pyrrolidin-3-yl]-2-(trifluoromethyl)benzenesulfonamide

In an analogous manner to example 688, tert-butyl(3S)-3-({[5-(phenylsulfonyl)-2-(trifluoromethyl)phenyl]sulfonyl}amino)pyrrolidine-1-carboxylate was used to prepare 5-(phenylsulfonyl)-N-[(3S)-pyrrolidin-3-yl]-2-(trifluoromethyl)benzenesulfonamide.

MS (ES+) m/z 434.7;

HPLC purity 97.2% at 210-370 nm, 6.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₇H₁₇F₃N₂O₄S₂+H+, 435.06546; found (ESI, [M+H]⁺), 435.0656.

### EXAMPLE 797

### 5-(phenylsulfonyl)-N-[(3R)-pyrrolidin-3-yl]-2-(trifluoromethyl)benzenesulfonamide

In an analogous manner to example 688, tert-butyl(3R)-3-({[5-(phenylsulfonyl)-2-(trifluoromethyl)phenyl]sulfonyl}amino)pyrrolidine-1-carboxylate was used to prepare 5-(phenylsulfonyl)-N-[(3R)-pyrrolidin-3-yl]-2-(trifluoromethyl)benzenesulfonamide.

MS (ES+) m/z 434.7;

HPLC purity 96.9% at 210-370 nm, 6.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₇H₁₇F₃N₂O₄S₂+H+, 435.06546; found (ESI, [M+H]⁺), 435.0658.

### EXAMPLE 798

### 2-methyl-5-(phenylsulfonyl)-N-(tetrahydro-2H-thiopyran-4-yl)benzenesulfonamide

Step 1: To a solution of tetrahydrothiopyran-4-one (3.0 g, 25.82 mmol) and 2,4-dimethoxybenzylamine (4.3 g, 25.82 mmol) in dichloroethane was added sodium triacetoxyborohydride (7.7 g, 36.15 mmol). The reaction was stirred overnight at room temperature under nitrogen. The reaction was quenched with a saturated aqueous bicarbonate solution and extracted. Dried with magnesium sulfate and concentrated down to give (2,4-dimethoxy-benzyl)-(tetrahydro-thiopyran-4-yl)-amine (6.7 g, 97%).

Step 2: To a solution of 5-benzenesulfonyl-2-methyl-benzenesulfonyl chloride (0.30 g, 0.90 mmol) in dichloromethane (12 mL) was added triethylamine (0.38 μL, 2.72 mmol) followed by (2,4-dimethoxy-benzyl)-(tetrahydro-thiopyran-4-yl)-amine (0.49 g, 1.81 mmol). The reaction was stirred overnight at room temperature under nitrogen. The following day the reaction was concentrated down onto silica gel and purified using automated chromatography with a gradient mobile phase consisting of ethyl acetate and hexane resulting in the isolation of 5-benzenesulfonyl-N-(2,4-dimethoxy-benzyl)-2-methyl-N-(tetrahydro-thiopyran-4-yl)-benzenesulfonamide.

Step 3: 5-Benzenesulfonyl-N-(2,4-dimethoxy-benzyl)-2-methyl-N-(tetrahydro-thiopyran-4-yl)-benzenesulfonamide was taken up in 5 mL of 6% trifluoroacetic acid/dichloromethane and was stirred overnight at room temperature under nitrogen. The reaction was extracted with water. The organic layer was washed with saturated solution of sodium bicarbonate and dried with magnesium sulfate. The reaction was concentrated down onto silica gel and purified using automated chromatography with a gradient mobile phase consisting of ethyl acetate and hexane resulting in the isolation of 2-methyl-5-(phenylsulfonyl)-N-(tetrahydro-2H-thiopyran-4-yl)benzenesulfonamide (0.31 g, 84%).

MS (ES+) m/z 411.7;

HRMS: calcd. for C₁₈H₂₁NO₄S₃+H⁺, 412.0711: found (ESI, [M+H]⁺), 412.0681.

### EXAMPLE 799

### N-[2-(Methylamino)ethyl]-5-(phenylsulfonyl)-2-(trifluoromethyl)benzenesulfonamide hydrochloride

Approximately 100 mL of a saturated solution of anhydrous hydrogen chloride in ethyl acetate was added to a solution of tert-butyl methyl[2-({[5-(phenylsulfonyl)-2-(trifluoromethyl)phenyl]sulfonyl}amino)ethyl]carbamate (2.8555 g, 5.46 mmol), prepared in Example 793, in 100 mL of ethyl acetate at room temperature. After the addition the reaction was stirred for 6 h. The solid was collected by filtration, rinsed with ethyl acetate and dried under reduced pressure to give N-[2-(methylamino)ethyl]-5-(phenylsulfonyl)-2-(trifluoromethyl)benzenesulfonamide hydrochloride (2.2506 g, 90%) as a white solid.

MS (ES−) m/z 420.6;

HRMS: calcd for C₁₆H₁₇F₃N₂O₄S₂+H⁺, 423.06546; found (ESI, [M+H]⁺), 423.0543.

### EXAMPLE 800

### N-[1-(4-benzoylbenzoyl)piperidin-4-yl]-5-(phenylsulfonyl)-2-(trifluoromethyl)benzenesulfonamide

A solution of 4-benzoyl-benzoyl chloride (34 mg, 0.14 mmol) in dichloromethane (1 mL) was added to a stirred dichloromethane solution of Example 688 (62 mg, 0.14 mmol) and triethylamine (0.28 mmol). The reaction was concentrated to dryness and immediately purified by flash column chromatography using an ethyl acetate hexane gradient (20-50%) resulting in the isolation of N-[1-(4-benzoylbenzoyl)piperidin-4-yl]-5-(phenylsulfonyl)-2-(trifluoromethyl)benzenesulfonamide (72 mg, 79%).

MS (ES+) m/z 656.6;

HPLC purity 93.4% at 210-370 nm, 10.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₃₂H₂₇F₃N₂O₆S₂+H+, 657.13354; found (ESI, [M+H]⁺), 657.1337.

### EXAMPLE 801

### N-[1-(3-benzoylbenzoyl)piperidin-4-yl]-5-(phenylsulfonyl)-2-(trifluoromethyl)benzenesulfonamide

A solution of 3-benzoyl-benzoyl chloride (34 mg, 0.14 mmol) in dichloromethane (1 mL) was added to a stirred dichloromethane solution of Example 688 (62 mg, 0.14 mmol) and triethylamine (0.28 mmol). The reaction was concentrated to dryness and immediately purified by flash column chromatography using an ethyl acetate hexane gradient (20-50%) resulting in the isolation of N-[1-(3-benzoylbenzoyl)piperidin-4-yl]-5-(phenylsulfonyl)-2-(trifluoromethyl)benzenesulfonamide (67 mg, 71%).

MS (ES+) m/z 656.7;

HPLC purity 95.1% at 210-370 nm, 10.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₃₂H₂₇F₃N₂O₆S₂+H+, 657.13354; found (ESI, [M+H]⁺), 657.1326.

### EXAMPLE 802

### 1-Isopropyl-4-(phenylsulfonyl)benzene

Step a

The title compound was prepared from 4-isopropylbenzenesulfonyl chloride (8.75 g, 40.0 mmol), benzene (20.0 mL, 200 mmol), and aluminum chloride (6.4 g, 48.0 mmol) according to the procedure and in the same manner as described in Example 678, step a. 1-Isopropyl-4-(phenylsulfonyl)benzene (10.32g, 99%) was obtained as a homogeneous oil which solidified on standing.

Step b

### 2-Isopropyl-5-(phenylsulfonyl)benzenesulfonyl chloride

1-Isopropyl-4-(phenylsulfonyl)benzene (10.4 g, 40.0 mmol) was heated with stirring at 60° C. for one hour under nitrogen with chlorosulfonic acid (26.5 mL, 400 mmol). The mixture was cooled to room temperature, poured slowly into a cold solution of 2N hydrochloric acid, and extracted with ethyl acetate (2×.). The organic phase was washed with a saturated, aqueous sodium chloride solution, dried over anhydrous sodium sulfate, filtered, and the solvent concentrated in vacuo to yield 2-isopropyl-5-(phenylsulfonyl)benzenesulfonyl chloride (3.3 g, 24%) as a homogeneous, colorless, crystalline solid.

Step c

### N-(1-Benzoylpiperidin-4-yl)-2-isopropyl-5-(phenylsulfonyl)benzenesulfonamide

A stirred solution of 2-isopropyl-5-(phenylsulfonyl)benzenesulfonyl chloride (0.18 g, 0.5 mmol) in dichloromethane (10 mL) was treated under nitrogen with (4-aminopiperidin-1-yl)(phenyl)methanone hydrochloride (0.18 g, 0.75 mmol) and a solution of diisopropylethylamine (0.26 g, 2.0 mmol) in dichloromethane. The reaction was stirred from 2-18 hours at room temperature. The crude product was purified by preparative liquid chromatography on a Biotage® 40 Si column of pre-packed silica gel (45 g), eluting with a gradient of 50%-100% methyl tert-butyl ether in hexane at a flow rate of 50 mL/min, to afford, after crystallization from diethyl ether-hexane, N-(1-benzoylpiperidin-4-yl)-2-isopropyl-5-(phenylsulfonyl)benzenesulfonamide (0.26 g, 76%), as a homogeneous, colorless, crystalline solid, m.p. 225° C.;

MS (+ESI), m/z: 526.7 [M+H]⁺;

HRMS: calcd for C₂₇H₃₀N₂O₅S₂+H+, 527.16689; found (ESI, [M+H]⁺), 527.1677;

HPLC purity 100% at 210-370 nm, 9.5 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 803

### 2-Isopropyl-N-[1-(2-methoxybenzoyl)piperidin-4-yl]-5-(phenylsulfonyl)benzenesulfonamide

The title compound was prepared from 2-isopropyl-5-(phenylsulfonyl)benzenesulfonyl chloride (0.18 g, 0.5 mmol), (4-aminopiperidin-1-yl)(2-methoxyphenyl)methanone hydrochloride (0.20 g, 0.75 mmol), and diisopropylethylamine (0.26 g, 2.0 mmol) according to the procedure and in the same manner as described in Example 802, step c. The crude product was purified by preparative liquid chromatography on a Biotage@ 40 Si column of pre-packed silica gel (45 g), eluting with a gradient of 75%-100% methyl tert-butyl ether in hexane at a flow rate of 50 mL/min to afford 2-isopropyl-N-[1-(2-methoxybenzoyl)piperidin-4-yl]-5-(phenylsulfonyl)benzenesulfonamide (0.21 g, 75%), as a homogeneous, amorphous solid, m.p. 160-162° C.;

MS (+ESI), m/z: 556.7 [M+H]⁺;

HRMS: calcd for C₂₈H₃₂N₂O₆S₂+H+, 557.17745; found (ESI, [M+H]⁺), 557.1782;

HPLC purity 99.3% at 210-370 nm, 9.5 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 804

### N-[1-(3-Fluorobenzoyl)piperidin-4-yl]-2-isopropyl-5-(phenylsulfonyl)benzenesulfonamide

The title compound was prepared from 2-isopropyl-5-(phenylsulfonyl)benzenesulfonyl chloride (0.18 g, 0.5 mmol), (4-aminopiperidin-1-yl)(3-fluorophenyl)methanone hydrochloride (0.19 g, 0.75 mmol), and diisopropylethylamine (0.26 g, 2.0 mmol) according to the procedure and in the same manner as described in Example 802, step c. The crude product was purified by preparative liquid chromatography on a Biotage® 40 Si column of pre-packed silica gel (45 g), eluting with a gradient of 50%-100% methyl tert-butyl ether in hexane at a flow rate of 50 mL/min to afford, after crystallization from diethyl ether-hexane, N-[1-(3-fluorobenzoyl)piperidin-4-yl]-2-isopropyl-5-(phenylsulfonyl)benzenesulfonamide (0.16 g, 59%), as a homogeneous, colorless, crystalline solid, m.p. 215-218° C.;

MS (+ESI), m/z: 544.7 [M+H]⁺;

HRMS: calcd for C₂₇H₂₉FN₂O₅S₂+H+, 545.15747; found (ESI, [M+H]⁺), 545.158;

HPLC purity 100% at 210-370 nm, 9.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 805

### N-[1-(4-Fluorobenzoyl)piperidin-4-yl]-2-isopropyl-5-(phenylsulfonyl)benzenesulfonamide

The title compound was prepared from 2-isopropyl-5-(phenylsulfonyl)benzenesulfonyl chloride (0.18 g, 0.5 mmol), (4-aminopiperidin-1-yl)(4-fluorophenyl)methanone hydrochloride (0.19 g, 0.75 mmol), and diisopropylethylamine (0.26 g, 2.0 mmol) according to the procedure and in the same manner as described in Example 802, step c. The crude product was purified by preparative liquid chromatography on a Biotage® 40 Si column of pre-packed silica gel (45 g), eluting with a gradient of 50%-100% methyl tert-butyl ether in hexane at a flow rate of 50 mL/min to afford, after crystallization from diethyl ether-hexane, N-[1-(4-fluorobenzoyl)piperidin-4-yl]-2-isopropyl-5-(phenylsulfonyl)benzenesulfonamide (0.18 g, 64%), as a homogeneous, colorless, crystalline solid, m.p. 218-220° C.;

MS (+ESI), m/z: 544.7 [M+H]⁺;

HRMS: calcd for C₂₇H₂₉FN₂O₅S₂+H+, 545.15747; found (ESI, [M+H]⁺), 545.1559;

HPLC purity 100% at 210-370 nm, 9.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 806

### N-[1-(2-Fluorobenzoyl)piperidin-4-yl]-2-isopropyl-5-(phenylsulfonyl)benzenesulfonamide

The title compound was prepared from 2-isopropyl-5-(phenylsulfonyl)benzenesulfonyl chloride (0.18 g, 0.5 mmol), (4-aminopiperidin-1-yl)(2-fluorophenyl)methanone hydrochloride (0.19 g, 0.75 mmol), and diisopropylethylamine (0.26 g, 2.0 mmol) according to the procedure and in the same manner as described in Example 802, step c. The crude product was purified by preparative liquid chromatography on a Biotage® 40 Si column of pre-packed silica gel (45 g), eluting with a gradient of 50%-100% methyl tert-butyl ether in hexane at a flow rate of 50 mL/min to afford, after crystallization from diethyl ether-hexane, N-[1-(2-fluorobenzoyl)piperidin-4-yl]-2-isopropyl-5-(phenylsulfonyl)benzenesulfonamide (0.18 g, 68%), as a homogeneous, colorless, crystalline solid, m.p. 210-212° C.;

MS (+ESI), m/z: 544.7 [M+H]⁺;

HRMS: calcd for C₂₇H₂₉FN₂O₅S₂+H+, 545.15747; found (ESI, [M+H]⁺), 545.1573;

HPLC purity 100% at 210-370 nm, 9.5 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 807

### 2-Isopropyl-5-(phenylsulfonyl)-N-{1-[4-(trifluoromethyl)benzoyl]piperidin-4-yl}benzenesulfonamide

The title compound was prepared from 2-isopropyl-5-(phenylsulfonyl)benzenesulfonyl chloride (0.18 g, 0.5 mmol), (4-aminopiperidin-1-yl)(4-(trifluoromethyl)phenyl)methanone hydrochloride (0.23 g, 0.75 mmol), and diisopropylethylamine (0.26 g, 2.0 mmol) according to the procedure and in the same manner as described in Example 802, step c. The crude product was purified by preparative liquid chromatography on a Biotage® 40 Si column of pre-packed silica gel (45 g), eluting with a gradient of 25%-100% methyl tert-butyl ether in hexane at a flow rate of 50 mL/min to afford 2-isopropyl-5-(phenylsulfonyl)-N-{1-[4-(trifluoromethyl)benzoyl]piperidin-4-yl}benzenesulfonamide (0.24 g, 79%), as a homogeneous, amorphous solid, m.p. 140-142° C.;

MS (+ESI), m/z: 594.7 [M+H]⁺;

HRMS: calcd for C₂₈H₂₉F₃N₂O₅S₂+H+, 595.15427; found (ESI, [M+H]⁺), 595.1538;

HPLC purity 99.3% at 210-370 nm, 10.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 808

### 2-Isopropyl-N-[1-(1-naphthoyl)piperidin-4-yl]-5-(phenylsulfonyl)benzenesulfonamide

The title compound was prepared from 2-isopropyl-5-(phenylsulfonyl)benzenesulfonyl chloride (0.18 g, 0.5 mmol), (4-aminopiperidin-1-yl)(naphthalen-1-yl)methanone hydrochloride (0.22 g, 0.75 mmol), and diisopropylethylamine (0.26 g, 2.0 mmol) according to the procedure and in the same manner as described in Example 802, step c. The crude product was purified by preparative liquid chromatography on a Biotage® 40 Si column of pre-packed silica gel (45 g), eluting with a gradient of 50%-100% methyl tert-butyl ether in hexane at a flow rate of 50 mL/min to afford 2-isopropyl-N-[1-(1-naphthoyl)piperidin-4-yl]-5-(phenylsulfonyl)benzenesulfonamide (0.27 g, 92%), as a homogeneous, amorphous solid, m.p. 163-165° C.;

MS (+ESI), m/z: 576.7 [M+H]⁺;

HRMS: calcd for C₃₁H₃₂N₂O₅S₂+H+, 577.18254; found (ESI, [M+H]⁺), 577.1828;

HPLC purity 99.0% at 210-370 nm, 10.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 809

### 2-Isopropyl-N-[1-(2-naphthoyl)piperidin-4-yl]-5-(phenylsulfonyl)benzenesulfonamide

The title compound was prepared from 2-isopropyl-5-(phenylsulfonyl)benzenesulfonyl chloride (0.18 g, 0.5 mmol), (4-aminopiperidin-1-yl)(naphthalen-2-yl)methanone hydrochloride (0.22 g, 0.75 mmol), and diisopropylethylamine (0.26 g, 2.0 mmol) according to the procedure and in the same manner as described in Example 802, step c. The crude product was purified by preparative liquid chromatography on a Biotage® 40 Si column of pre-packed silica gel (45 g), eluting with a gradient of 25%-100% methyl tert-butyl ether in hexane at a flow rate of 50 mL/min to afford 2-isopropyl-N-[1-(2-naphthoyl)piperidin-4-yl]-5-(phenylsulfonyl)benzenesulfonamide (0.24 g, 84%), as a homogeneous, amorphous solid, m.p. 130-132° C.;

MS (+ESI), m/z: 576.7 [M+H]+;

HRMS: calcd for C₃₁H₃₂N₂O₅S₂+H+, 577.18254; found (ESI, [M+H]⁺), 577.183;

HPLC purity 99.7% at 210-370 nm, 10.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 810

### N-[1-(3-Cyanobenzoyl)piperidin-4-yl]-2-isopropyl-5-(phenylsulfonyl)benzenesulfonamide

The title compound was prepared from 2-isopropyl-5-(phenylsulfonyl)benzenesulfonyl chloride (0.18 g, 0.5 mmol), 3-(4-aminopiperidine-1-carbonyl)benzonitrile hydrochloride (0.20 g, 0.75 mmol), and diisopropylethylamine (0.26 g, 2.0 mmol) according to the procedure and in the same manner as described in Example 802, step c. The crude product was purified by preparative liquid chromatography on a Biotage® 40 Si column of pre-packed silica gel (45 g), eluting with a gradient of 50%-100% methyl tert-butyl ether in hexane at a flow rate of 50 mL/min to afford N-[1-(3-cyanobenzoyl)piperidin-4-yl]-2-isopropyl-5-(phenylsulfonyl)benzenesulfonamide (0.19 g, 69%), as a homogeneous, amorphous solid, m.p. 120-122° C.;

MS (+ESI), m/z: 551.7 [M+H]⁺;

HRMS: calcd for C₂₈H₂₉N₃O₅S₂+H+, 552.16214; found (ESI, [M+H]⁺), 552.1634;

HPLC purity 100% at 210-370 nm, 9.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 811

### N-[1-(4-Cyanobenzoyl)piperidin-4-yl]-2-isopropyl-5-(phenylsulfonyl)benzenesulfonamide

The title compound was prepared from 2-isopropyl-5-(phenylsulfonyl)benzenesulfonyl chloride (0.18 g, 0.5 mmol), 4-(4-aminopiperidine-1-carbonyl)benzonitrile hydrochloride (0.20 g, 0.75 mmol), and diisopropylethylamine (0.26 g, 2.0 mmol) according to the procedure and in the same manner as described in Example 802, step c. The crude product was purified by preparative liquid chromatography on a Biotage® 40 Si column of pre-packed silica gel (45 g), eluting with a gradient of 25%-100% methyl tert-butyl ether in hexane at a flow rate of 50 mL/min to afford N-[1-(4-cyanobenzoyl)piperidin-4-yl]-2-isopropyl-5-(phenylsulfonyl)benzenesulfonamide (0.21 g, 77%), as a homogeneous, amorphous solid, m.p. 136-138° C.;

MS (+ESI), m/z: 551.7 [M+H]⁺;

HRMS: calcd for C₂₈H₂₉N₃O₅S₂+H+, 552.16214; found (ESI, [M+H]⁺), 552.1635;

HPLC purity 100% at 210-370 nm, 9.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 812

### N-[1-(4-tert-Butylbenzoyl)piperidin-4-yl]-2-isopropyl-5-(phenylsulfonyl)benzenesulfonamide

The title compound was prepared from 2-isopropyl-5-(phenylsulfonyl)benzenesulfonyl chloride (0.18 g, 0.5 mmol), (4-aminopiperidin-1-yl)(4-tert-butylphenyl)methanone hydrochloride (0.22 g, 0.75 mmol), and diisopropylethylamine (0.26 g, 2.0 mmol) according to the procedure and in the same manner as described in Example 802, step c. The crude product was purified by preparative liquid chromatography on a Biotage® 40 Si column of pre-packed silica gel (45 g), eluting with a gradient of 10%-100% methyl tert-butyl ether in hexane at a flow rate of 50 mL/min to afford, after crystallization from diethyl ether-hexane, N-[1-(4-tert-butylbenzoyl)piperidin-4-yl]-2-isopropyl-5-(phenylsulfonyl)benzenesulfonamide (0.28 g, 95%), as a homogeneous, colorless, crystalline solid, m.p. 150-152° C.;

MS (+ESI), m/z: 582.8 [M+H]⁺;

HRMS: calcd for C₃₁H₃₈N₂O₅S₂+H+, 583.22949; found (ESI, [M+H]⁺), 583.2277;

HPLC purity 99.4% at 210-370 nm, 10.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 813

### N-[1-(2-Ethoxy-1-naphthoyl)piperidin-4-yl]-2-isopropyl-5-(phenylsulfonyl)benzenesulfonamide

The title compound was prepared from 2-isopropyl-5-(phenylsulfonyl)benzenesulfonyl chloride (0.18 g, 0.5 mmol), (4-aminopiperidin-1-yl)(2-ethoxynaphthalen-1-yl)methanone hydrochloride (0.25 g, 0.75 mmol), and diisopropylethylamine (0.26 g, 2.0 mmol) according to the procedure and in the same manner as described in Example 802, step c. The crude product was purified by preparative liquid chromatography on a Biotage® 40 Si column of pre-packed silica gel (45 g), eluting with a gradient of 10%-100% methyl tert-butyl ether in hexane at a flow rate of 50 mL/min to afford, after crystallization from diethyl ether-hexane, N-[1-(2-ethoxy-1-naphthoyl)piperidin-4-yl]-2-isopropyl-5-(phenylsulfonyl)benzenesulfonamide (0.28 g, 89%), as a homogeneous, colorless, crystalline solid, m.p. 164-166° C.;

MS (+ESI), m/z: 620.8 [M+H]⁺;

HRMS: calcd for C₃₃H₃₆N₂O₆S₂+H+, 621.20875; found (ESI, [M+H]⁺), 621.2089;

HPLC purity 100% at 210-370 nm, 10.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 814

### 5-[(4-hydroxyphenyl)sulfonyl]-N-(2-pyridin-3-ylethyl)-2-(trifluoromethyl)benzenesulfonamide

Step 1: Following the same procedure described on example 677 (Step 2), 2-chloro-5-fluoronitrobenzene was used to prepare 2-nitro-4-fluorobenzotrifluoride.

Step 2: In an analogous manner to example 743 (Step 2), 4-methoxybenzenethiol was used to prepare 5-(4-methoxyphenylsulfonyl)-2-trifluoromethyl-nitrobenzene.

Step 3: In an analogous manner to example 677 (Step 3), 5-(4-methoxyphenylsulfonyl)-2-trifluoromethyl-nitrobenzene was used to prepare 5-(4-methoxyphenylsulfonyl)-2-trifluoromethylaniline.

Step 4: In an analogous manner to example 677 (Step 4), 5-(4-methoxyphenylsulfonyl)-2-trifluoromethylaniline was used to prepare 2-trifluoromethyl-5-(4-methoxyphenylsulfonyl)-benzenesulfonyl chloride.

Step 5: In an analogous manner to example 435, 2-trifluoromethyl-5-(4-methoxyphenylsulfonyl)-benzenesulfonyl chloride and 3-(2-aminoethyl)pyridine were used to prepare 5-[(4-methoxyphenyl)sulfonyl]-N-(2-pyridin-3-ylethyl)-2-(trifluoromethyl)benzenesulfonamide.

Step 6: In an analogous manner to example 744, 5-[(4-methoxyphenyl)sulfonyl]-N-(2-pyridin-3-ylethyl)-2-(trifluoromethyl)benzenesulfonamide, was used to prepare 5-[(4-hydroxyphenyl)sulfonyl]-N-(2-pyridin-3-ylethyl)-2-(trifluoromethyl)benzenesulfonamide.

MS (ES+) m/z 486.6;

HPLC purity 95.7% at 210-370 nm, 8.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₀H₁₇F₃N₂O₅S₂+H+, 487.06037; found (ESI, [M+H]⁺), 487.0609.

### EXAMPLE 815

### 5-[(4-fluorophenyl)sulfonyl]-2-isopropyl-N-(tetrahydro-2H-thiopyran-4-yl)benzenesulfonamide

Step 1: To a solution of tetrahydrothiopyran (3.0 g, 25.82 mmol) and 2,4-dimethoxybenzylamine (4.3 g, 25.82 mmol) in dichloromethane (100 mL) was added sodium triacetoxyborohydride (7.7 g, 36.15 mmol). The reaction was stirred overnight at room temperature under nitrogen. The reaction was quenched with a saturated aqueous bicarbonate solution and extracted. Dried with magnesium sulfate and concentrated to give N-(2,4-dimethoxybenzyl)tetrahydro-2H-thiopyran-4-amine (6.7 g, 97%).

Step 2: To a solution of 5-(4-fluorophenylsulfonyl)-2-isopropylbenzene-1-sulfonyl chloride (350 mg, 0.93 mmol) in dichloromethane 10 mL) was added triethylamine (388 μL, 2.79 mmol) followed by N-(2,4-dimethoxybenzyl)tetrahydro-2H-thiopyran-4-amine (497 mg, 1.86 mmol). The reaction was stirred overnight at room temperature. The following day the reaction was concentrated down onto silica gel and purified using automated chromatography with a gradient mobile phase consisting of ethyl acetate and hexane resulting in the isolation of N-(2,4-dimethoxybenzyl)-5-(4-fluorophenylsulfonyl)-2-isopropyl-N-(tetrahydro-2H-thiopyran-4-yl)benzenesulfonamide (504 mg, 89%).

Step 3: N-(2,4-dimethoxybenzyl)-5-(4-fluorophenylsulfonyl)-2-isopropyl-N-(tetrahydro-2H-thiopyran-4-yl)benzenesulfonamide (504 mg, 0.83 mmol) was dissolved in 6% trifluoroacetic acid/dichloromethane (6 mL) and stirred overnight at room temperature under nitrogen. Saturated bicarbonate solution was added and the reaction was extracted. Dried with magnesium sulfate and concentrated onto silica gel and purified using automated chromatography with a gradient mobile phase consisting of ethyl acetate and hexane resulting in the isolation of 5-[(4-fluorophenyl)sulfonyl]-2-isopropyl-N-(tetrahydro-2H-thiopyran-4-yl)benzenesulfonamide (350 mg, 92%).

MS (ES+) m/z 456.

### EXAMPLE 816

### 5-[(4-fluorophenyl)sulfonyl]-N-[2-(1H-imidazol-1-yl)ethyl]-2-(trifluoromethyl)benzenesulfonamide

Step 1: 1-chloro-4-(4-fluorophenylsulfonyl)-2-nitrobenzene (L31023-148). A mixture of 4-chloro-3-nitrobenzenesulfonyl chloride (25.13 g, 98.1 mmol), fluorobenzene (50 ml, 533 mmol) and aluminum chloride (15.71 g, 118 mmol) refluxed under nitrogen overnight. The next morning the reaction was allowed to cool to R.T. and then was transferred to a 1 L beaker where the reaction was mixed with ice for ten minutes. The contents of the beaker was partitioned between ethyl acetate and water. The ethyl acetate layer was separated, dried (anhydrous MgSO₄) and the solvent removed under reduced pressure to-give 1-chloro-4-(4-fluorophenylsulfonyl)-2-nitrobenzene (29.12 g, 93.9%).

Step 2: 4-(4-fluorophenylsulfonyl)-2-nitro-1-(trifluoromethyl)benzene (L31023-152). To a flame dried flask the 1-chloro-4-(4-fluorophenylsulfonyl)-2-nitrobenzene (29.12 g, 92.3 mmol) prepared in the previous step was taken up in 200 mL of anh. N,N-Dimethylformamide and was allowed stir under nitrogen at ice bath temperature. To the stirring reaction at ice bath temp., copper nanopowder (25.24 g, 0.4 mol) and activated carbon (13.67 g, 1.1 mol) was added to the reaction stirred for five minutes to which dibromodifluoromethane (19.8 ml, 0.2 mol) was slowly syringed into the reaction. After the complete addition of the dibromodifluoromethane, the reaction was heated to 100° C. for five hours and then allowed to cool to R.T. The contents of the flask was filtered though a plug of celite and the filtrate was then partitioned between ethyl acetate and ammonium chloride/water once. The organic layer was washed twice more with water. The ethyl acetate layer was separated, dried (anhydrous MgSO₄) and the solvent removed under reduced pressure to give 4-(4-fluorophenylsulfonyl)-2-nitro-1-(trifluoromethyl)benzene (24.3 g, 75.3%).

Step 3: 5-(4-fluorophenylsulfonyl)-2-(trifluoromethyl)aniline (L31023-153). A mixture of 4-(4-fluorophenylsulfonyl)-2-nitro-1-(trifluoromethyl)benzene (24.3 g, 69.6 mmol) and tin(II) chloride (65.9 g, 0.4 mol) was taken up in 1:16 water/methanol was refluxed for three days. The reaction was concentrated and then was partitioned between ethyl acetate and 2N hydrochloric acid. The ethyl acetate layer was separated, dried (anhydrous MgSO₄) and the solvent removed under reduced pressure to give 25.6 g of a brown solid. The solid was purified on the Isco using a 330 g column and a gradient of hexane/methylene chloride to give 5-(4-fluorophenylsulfonyl)-2-(trifluoromethyl)aniline.

Step 4: 5-(4-fluorophenylsulfonyl)-2-(trifluoromethyl)benzene-1-sulfonyl chloride (L31023-160). 5-(4-fluorophenylsulfonyl)-2-(trifluoromethyl)aniline(3.79 g, 11.9 mmol) prepared in the previous step was taken up in 100 ml of acetonitrile and was cooled to ice bath temperature. To the chilled flask, 10 ml of acetic acid, 10 ml of hydrochloric acid and then sodium nitrite (0.98 g, 14.2 mmol) taken up in 2 ml of water were slowly syringed into the flask. The mixture was allowed to stir at ice bath temperature for one hour. Sulfur dioxide was then bubbled into the flask for fifteen minutes and then copper(II) chloride dihydrate taken up in 2 ml of water was syringed into the flask and was allowed to stir at ice bath temp. for three hours. The mixture was allowed to warm to room temp. and then was partitioned between ethyl acetate and water. The ethyl acetate layer was separated, dried (anhydrous MgSO₄) and the solvent removed under reduced pressure to provide an orange oil which was purified on the Isco using a 330 g column and a gradient of hexane/methylene chloride to give 5-(4-fluorophenylsulfonyl)-2-(trifluoromethyl)benzene-1-sulfonyl chloride (2.2234 g, 46.5%).

Step 5: 5-[(4-fluorophenyl)sulfonyl]-N-[2-(1H-imidazol-1-yl)ethyl]-2-(trifluoromethyl)benzenesulfonamide). A mixture of sodium carbonate (0.1601 g, 1.5 mmol) in 1 ml of water and 2-(1H-imidazol-1-yl)ethyl amine (0.092, 0.5 mmol) in 3 ml of acetonitrile was treated with the mixture of 5-(4-fluorophenylsulfonyl)-2-(trifluoromethyl)benzene-1-sulfonyl chloride and 5-(2-fluorophenylsulfonyl)-2-(trifluoromethyl)benzene-1-sulfonyl chloride (0.2514 g, 0.6 mmol) prepared in the previous step in 10 ml of acetonitrile. The reaction was allowed to stir overnight at R. T. and then was partitioned between ethyl acetate and 2N hydrochloric acid. The ethyl acetate layer was separated, dried (anhydrous MgSO₄) and the solvent removed under reduced pressure to give approximately 50 mg of the isomeric mixture that was dissolved in 3 mL of methanol/acetonitrile. 200 □L of the resulting solution was repetitively injected onto the Supercritical Fluid Chromatography (SFC) instrument, and the baseline resolved isomers were separately collected using the conditions described below to give the title compound 5-[(4-fluorophenyl)sulfonyl]-N-[2-(1H-imidazol-1-yl)ethyl]-2-(trifluoromethyl)benzenesulfonamide (31.2 mg, 10%) as a white solid.

| | |
|---|---|
| SFC Instrument: | Berger MultiGram Prep SFC |
| Column: | Ethylpyridine; 10□m; 250 mm L × 20 mm ID |
| Column temperature: | 35° C. |
| SFC Modifier: | 15% MeOH with 0.2% DEA/85% CO2 |
| Flow rate: | 50 mL/min |
| Outlet Pressure: | 100 bar |
| Detector: | UV at 220 nm |

MS (ES+) m/z 477.6;

HPLC purity analytical SFc purity check after prep SFC separation of regioisomers peak 1=97.5% peak 2=96.8% minor impurities are of unknown composition; Ethyl pyridine 4.6×250 mm column, 2 mL/min, 15% MeOH/85% CO2.

HPLC purity 100% at 210-370 nm, 7.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₈H₁₅F₄N₃O₄S₂+H+, 478.05129; found (ESI, [M+H]+), 478.0507.

### EXAMPLE 817

### 5-[(4-fluorophenyl)sulfonyl]-N-(tetrahydro-2H-pyran-4-yl)-2-(trifluoromethyl)benzenesulfonamide

In an analogous manner to example 765, a mixture of 5-[(4-fluorophenyl)sulfonyl]-N-[2-(1H-imidazol-1-yl)ethyl]-2-(trifluoromethyl)benzenesulfonamide) and 5-(2-fluorophenylsulfonyl)-2-(trifluoromethyl)benzene-1-sulfonyl chloride and 4-Aminotetrahydropyran to give approximately 85 mg of the isomeric mixture that was dissolved in 2.5 mL of methanol/acetonitrile. 250 μL of the resulting solution was repetitively injected onto the Supercritical Fluid Chromatography (SFC) instrument, and the baseline resolved isomers were separately collected using the conditions described below to give the title compound of 5-[(4-fluorophenyl)sulfonyl]-N-(tetrahydro-2H-pyran-4-yl)-2-(trifluoromethyl)benzenesulfonamide (22.2 mg, 8%) as a white solid.

| | |
|---|---|
| SFC Instrument: | Berger MultiGram Prep SFC |
| Column: | Ethylpyridine; 10 μm; 250 mm L × 20 mm ID |
| Column temperature: | 35° C. |
| SFC Modifier: | 15% MeOH/85% CO2 |
| Flow rate: | 50 mL/min |
| Outlet Pressure: | 100 bar |
| Detector: | UV at 220 nm |

MS (ES−) m/z 465.6;

HPLC purity The peak purity is checked by analytical SFC: peak 1—>99.9% peak 2—>99.9%; Ethyl Pyridine (4.6×250 mm) column, 2 mL/min, 15% MeOH/85% CO2.

HPLC purity 100% at 210-370 nm, 8.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 818

### tert-butyl 4-({[5-[(4-fluorophenyl)sulfonyl]-2-(trifluoromethyl)phenyl]sulfonyl}amino)piperidine-1-carboxylate

In an analogous manner to example 765, a mixture of 5-[(4-fluorophenyl)sulfonyl]-N-[2-(1H-imidazol-1-yl)ethyl]-2-(trifluoromethyl)benzenesulfonamide) and 5-(2-fluorophenylsulfonyl)-2-(trifluoromethyl)benzene-1-sulfonyl chloride and 4-Amino-piperidine-1-carboxylic acid tert-butyl to give approximately 40 mg of the isomeric mixture that was dissolved in 2 mL of methanol/acetonitrile. 250 μL of the resulting solution was repetitively injected onto the Supercritical Fluid Chromatography (SFC) instrument, and the baseline resolved isomers were separately collected using the conditions described below to give the title compound tert-butyl 4-({[5-[(4-fluorophenyl)sulfonyl]-2-(trifluoromethyl)phenyl]sulfonyl}amino)piperidine-1-carboxylate (27.6 mg, 8%) as a white solid.

| | |
|---|---|
| SFC Instrument: | Berger MultiGram Prep SFC |
| Column: | Ethylpyridine; 10 μm; 250 mm L × 20 mm ID |
| Column temperature: | 35° C. |
| SFC Modifier: | 15% MeOH/85% CO2 |
| Flow rate: | 50 mL/min |
| Outlet Pressure: | 100 bar |
| Detector: | UV at 220 nm |

MS (ESI−) m/z 565;

HPLC purity The purity was checked by analytical SFC: peak 1—>99.9% peak 2—>99.9%; Ethyl Pyridine (4.6×250 mm) column, 2 mL/min, 15% MeOH/85% CO₂.

HPLC purity 100% at 210-370 nm, 10.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₃H₂₆F₄N₂O₆S₂+H+, 567.12412; found (ESI, [M−H—C4H8]+) 511.0644.

### EXAMPLE 819

### N-Methyl-N-[2-({[5-(phenylsulfonyl)-2-(trifluoromethyl)phenyl]sulfonyl}amino)ethyl]benzamide

Benzoyl chloride (38 μL, 0.327 mmol) was added under nitrogen to a solution of N-[2-(methylamino)ethyl]-5-(phenylsulfonyl)-2-(trifluoromethyl)benzenesulfonamide hydrochloride (150.8 mg, 0.329 mmol), prepared in Example 799, and triethylamine (229 μL, 1.64 mmol) in 20 mL of methylene chloride at room temperature. After the addition the reaction was stirred at room temperature for 19 h. The reaction was extracted two times with 2 N HCl, dried (anhydrous magnesium sulfate) and the solvent removed under reduced pressure to give 184.1 mg of a solid. Purification of the solid on a 12 g Redi Sep Fash Column (silica gel) using a gradient of 100% methylene chloride to 40% ethyl acetate-methylene chloride as the eluent gave N-methyl-N-[2-({[5-(phenylsulfonyl)-2-(trifluoromethyl)phenyl]sulfonyl}amino)ethyl]benzamide (156.3 mg, 91%) as a white solid.

MS (ES+) m/z 526.6;

HRMS: calcd for C₂₃H₂₁F₃N₂O₅S₂+H⁺, 527.09167; found (ESI, [M+H]⁺), 527.0908;

Anal. Calcd for C₂₃H₂₁F₃N₂O₅S₂. 0.20 CH₂Cl₂: C, 51.27; H, 3.97; N, 5.15. Found C, 52.54; H, 3.87; N, 5.17.

### EXAMPLE 820

### N-(2-cyanoethyl)-5-[(4-methoxyphenyl)sulfonyl]-2-(trifluoromethyl)benzenesulfonamide

Step 1: Following the same procedure described on example 677 (Step 2), 2-chloro-5-fluoronitrobenzene was used to prepare 2-nitro-4-fluorobenzotrifluoride.

Step 2: In an analogous manner to example 743 (Step 2), 4-methoxybenzenethiol was used to prepare 5-(4-methoxyphenylsulfonyl)-2-trifluoromethyl-nitrobenzene.

Step 3: In an analogous manner to example 677 (Step 3), 5-(4-methoxyphenylsulfonyl)-2-trifluoromethyl-nitrobenzene was used to prepare 5-(4-methoxyphenylsulfonyl)-2-trifluoromethylaniline.

Step 4: In an analogous manner to example 677 (Step 4), 5-(4-methoxyphenylsulfonyl)-2-trifluoromethylaniline was used to prepare 2-trifluoromethyl-5-(4-methoxyphenylsulfonyl)-benzenesulfonyl chloride.

Step 5: In an analogous manner to example 435, 2-trifluoromethyl-5-(4-methoxyphenylsulfonyl)-benzenesulfonyl chloride and aminopropionitrile were used to prepare N-(2-cyanoethyl)-5-[(4-methoxyphenyl)sulfonyl]-2-(trifluoromethyl)benzenesulfonamide.

MS (ES+) m/z 448.6;

HPLC purity 94.6% at 210-370 nm, 8.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₇H₁₅F₃N₂O₅S₂+H+, 449.04472; found (ESI, [M+H]⁺), 449.0429

### EXAMPLE 821

### N-(2-cyanoethyl)-5-[(4-hydroxyphenyl)sulfonyl]-2-(trifluoromethyl)benzenesulfonamide

In an analogous manner to example 744, N-(2-cyanoethyl)-5-[(4-methoxyphenyl)sulfonyl]-2-(trifluoromethyl)benzenesulfonamide was used to prepare N-(2-cyanoethyl)-5-[(4-hydroxyphenyl)sulfonyl]-2-(trifluoromethyl)benzenesulfonamide

MS (ES−) m/z 432.5;

HPLC purity 85.6% at 210-370 nm, 7.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₆H₁₃F₃N₂O₅S₂+H+, 435.02907; found (ESI, [M+H]⁺), 435.0308.

### EXAMPLE 822

### 4-Methoxy-N-methyl-N-[2-({[5-(phenylsulfonyl)-2-(trifluoromethyl)phenyl]sulfonyl}amino)ethyl]benzamide

In an analogous manner as described in Example 819, and replacing benzoyl chloride with 4-methoxy-benzoyl chloride, gave 4-methoxy-N-methyl-N-[2-({[5-(phenylsulfonyl)-2-(trifluoromethyl)phenyl]sulfonyl}amino)ethyl]benzamide (176.5 mg, 97%) as a white solid.

MS (ES+) m/z 556.6;

HRMS: calcd for C₂₄H₂₃F₃N₂O₆S₂+H+, 557.10224; found (ESI, [M+H]+), 557.1029;

Anal. Calcd for C₂₄H₂₃F₃N₂O₆S₂: C, 51.79; H, 4.17; N, 5.03. Found: C, 52.03; H, 4.13; N, 4.74.

### EXAMPLE 823

### N-(2-hydroxy-2,3-dihydro-1H-inden-1-yl)-5-(phenylsulfonyl)-2-(trifluoromethyl)benzenesulfonamide

In an analogous manner to example 765, 5-(phenylsulfonyl)-2-(trifluoromethyl)benzenesulfonyl chloride and 1-amino-2,3-dihydro-1H-inden-2-ol was used to prepare the title compound N-(2-hydroxy-2,3-dihydro-1H-inden-1-yl)-5-(phenylsulfonyl)-2-(trifluoromethyl)benzenesulfonamide (150.2 mg, 57%) as a white solid.

MS (ES−) m/z 495.7;

HPLC purity 94.4% at 210-370 nm, 9.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₂H₁₈F₃NO₅S₂+H+, 498.06512; found (ESI, [M+H]+), 498.0664.

### EXAMPLE 824

### 5-[(2-fluorophenyl)sulfonyl]-N-(tetrahydro-2H-pyran-4-yl)-2-(trifluoromethyl)benzenesulfonamide

In an analogous manner to example 765, 5-[(4-fluorophenyl)sulfonyl]-N-[2-(1H-imidazol-1-yl)ethyl]-2-(trifluoromethyl)benzenesulfonamide) and 5-(2-fluorophenylsulfonyl)-2-(trifluoromethyl)benzene-1-sulfonyl chloride and 4-aminotetrahydropyran to give approximately 85 mg of the isomeric mixture that was dissolved in 2.5 mL of methanol/acetonitrile. 250 μL of the resulting solution was repetitively injected onto the Supercritical Fluid Chromatography (SFC) instrument, and the baseline resolved isomers were separately collected using the conditions described below to give the title compound 5-[(4-fluorophenyl)sulfonyl]-N-(tetrahydro-2H-pyran-4-yl)-2-(trifluoromethyl)benzenesulfonamide (22.2 mg, 8%) as a white solid.

| | |
|---|---|
| SFC Instrument: | Berger MultiGram Prep SFC |
| Column: | Ethylpyridine; 10 μm; 250 mm L × 20 mm ID |
| Column temperature: | 35° C. |
| SFC Modifier: | 15% MeOH/85% CO2 |
| Flow rate: | 50 mL/min |
| Outlet Pressure: | 100 bar |
| Detector: | UV at 220 nm |

MS (ES+) m/z 467.6;

HPLC purity 100% at 210-370 nm, 8.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 825

### 5-(phenylsulfonyl)-N-[1-(phenylsulfonyl)piperidin-4-yl]-2-(trifluoromethyl)benzenesulfonamide

In an analogous manner to Example 462, 5-(phenylsulfonyl)-N-piperidin-4-yl-2-(trifluoromethyl)benzenesulfonamide and benzenesulfonyl chloride were used to prepare 5-(phenylsulfonyl)-N-[1-(phenylsulfonyl)piperidin-4-yl]-2-(trifluoromethyl)benzenesulfonamide.

MS (ES+) m/z 588.5;

HPLC purity 98.0% at 210-370 nm, 9.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₄H₂₃F₃N₂O₆S₃+H+, 589.07431; found (ESI, [M+H]⁺), 589.0731.

### EXAMPLE 826

### N-{2-[(anilinocarbonyl)(methyl)amino]ethyl}-5-(phenylsulfonyl)-2-(trifluoromethyl)benzenesulfonamide

To a stirred suspension of N-[2-(methylamino)ethyl]-5-(phenylsulfonyl)-2-(trifluoromethyl)benzenesulfonamide hydrochloride (0.16 g, 0.35 mmol) from Example 799 in a solution of phenyl isocyanate (42 mg, 0.35 mmol) in CH₂Cl₂ (8 mL) under N₂ at room temperature was added diisopropylethylamine (70 μL, 52 mg, 0.40 mmol). The mixture was stirred at room temperature for 18 hours. It was loaded directly onto a silica gel column and eluted with a gradient of hexane and ethyl acetate to give 0.18 g (95%) of N-{2-[(anilinocarbonyl)(methyl)amino]ethyl}-5-(phenylsulfonyl)-2-(trifluoromethyl)benzenesulfonamide.

MS (ES+) m/z 541.6;

HRMS: calcd for C₂₃H₂₂F₃N₃O₅S₂+H+, 542.10257; found (ESI, [M+H]⁺), 542.1027.

### EXAMPLE 827

### N-(2-{methyl[(pyridin-3-ylamino)carbonyl]amino}ethyl)-5-(phenylsulfonyl)-2-(trifluoromethyl)benzenesulfonamide

In an analogous manner to Example 826, N-[2-(methylamino)ethyl]-5-(phenylsulfonyl)-2-(trifluoromethyl)benzenesulfonamide hydrochloride from Example 799 and pyridine-3-isocyanate were used to prepare N-(2-{methyl[(pyridin-3-ylamino)carbonyl]amino}ethyl)-5-(phenylsulfonyl)-2-(trifluoromethyl)benzenesulfonamide.

MS (ES+) m/z 543.2;

HRMS: calcd for C₂₂H₂₁F₃N₄O₅S₂+H+, 543.09782; found (ESI, [M+H]⁺), 543.0983.

### EXAMPLE 828

### N-{2-[{[(2,4-dimethoxyphenyl)amino]carbonyl}(methyl)amino]ethyl}-5-(phenylsulfonyl)-2-(trifluoromethyl)benzenesulfonamide

In an analogous manner to example 826, N-[2-(methylamino)ethyl]-5-(phenylsulfonyl)-2-(trifluoromethyl)benzenesulfonamide hydrochloride from Example 799 and 2,4-dimethoxyphenyl isocyanate were used to prepare N-{2-[{[(2,4-dimethoxyphenyl)amino]carbonyl}(methyl)amino]ethyl}-5-(phenylsulfonyl)-2-(trifluoromethyl)benzenesulfonamide.

MS (ES+) m/z 601.6;

HRMS: calcd for C₂₅H₂₆F₃N₃O₇S₂+H+, 602.12370; found (ESI, [M+H]⁺), 602.1265.

### EXAMPLE 829

### N-{2-[[(tert-butylamino)carbonyl](methyl)amino]ethyl}-5-(phenylsulfonyl)-2-(trifluoromethyl)benzenesulfonamide

In an analogous manner to Example 826, N-[2-(methylamino)ethyl]-5-(phenylsulfonyl)-2-(trifluoromethyl)benzenesulfonamide hydrochloride from Example 799 and 4-(tert-butyl)phenyl isocyanate were used to prepare N-{2-[[(tert-butylamino)carbonyl](methyl)amino]ethyl}-5-(phenylsulfonyl)-2-(trifluoromethyl)benzenesulfonamide.

MS (ES+) m/z 521.7;

HRMS: calcd for C₂₁H₂₆F₃N₃O₅S₂+H+, 522.13387; found (ESI, [M+H]⁺), 522.1318.

### EXAMPLE 830

### N-{2-[{[(4-methoxyphenyl)amino]carbonyl}(methyl)amino]ethyl}-5-(phenylsulfonyl)-2-(trifluoromethyl)benzenesulfonamide

In an analogous manner to Example 826, N-[2-(methylamino)ethyl]-5-(phenylsulfonyl)-2-(trifluoromethyl)benzenesulfonamide hydrochloride from Example 799 and 4-methoxyphenyl isocyanate were used to prepare N-{2-[{[(4-methoxyphenyl)amino]carbonyl}(methyl)amino]ethyl}-5-(phenylsulfonyl)-2-(trifluoromethyl)benzenesulfonamide.

MS (ES+) m/z 571.6;

HRMS; calcd for C₂₄H₂₄F₃N₃O₆S₂+H+, 572.11314; found (ESI, [M+H]⁺), 572.1152.

### EXAMPLE 831

### N-(2-[[(butylamino)carbonyl](methyl)amino]ethyl}-5-(phenylsulfonyl)-2-(trifluoromethyl)benzenesulfonamide

In an analogous manner to Example 826, N-[2-(methylamino)ethyl]-5-(phenylsulfonyl)-2-(trifluoromethyl)benzenesulfonamide hydrochloride from Example 799 and butyl isocyanate were used to prepare N-{2-[[(butylamino)carbonyl](methyl)amino]ethyl}-5-(phenylsulfonyl)-2-(trifluoromethyl)benzenesulfonamide.

MS (ES+) m/z 521.7;

HRMS: calcd for C₂₁H₂₆F₃N₃O₅S₂+H+, 522.13387; found (ESI, [M+H]⁺), 522.1337.

### EXAMPLE 832

### N-{2-[{[(2,4-difluorophenyl)amino]carbonyl}(methyl)amino]ethyl}-5-(phenylsulfonyl)-2-(trifluoromethyl)benzenesulfonamide

In an analogous manner to Example 826, N-[2-(methylamino)ethyl]-5-(phenylsulfonyl)-2-(trifluoromethyl)benzenesulfonamide hydrochloride from Example 799 and 2,4-difluorophenyl isocyanate were used to prepare N-{2-[{[(2,4-difluorophenyl)amino]carbonyl}(methyl)amino]ethyl}-5-(phenylsulfonyl)-2-(trifluoromethyl)benzenesulfonamide.

MS (ES+) m/z 577.6;

HRMS: calcd for C₂₃H₂₀F₅N₃O₅S₂+H+, 578.08373; found (ESI, [M+H]⁺), 578.0847.

### EXAMPLE 833

### N-methyl-N-[2-({[5-(phenylsulfonyl)-2-(trifluoromethyl)phenyl]sulfonyl}amino)ethyl]pyrrolidine-1-carboxamide

To a stirred suspension of N-[2-(methylamino)ethyl]-5-(phenylsulfonyl)-2-(trifluoromethyl)benzenesulfonamide hydrochloride (0.16 g, 0.35 mmol) from Example 799 in a solution of 1-pyrrolidinecarbonyl chloride (39 μL, 47 mg, 0.35 mmol) in CH₂Cl₂ (8 mL) under N₂ at room temperature was added diisopropylethylamine (0.26 mL, 0.19 g, 1.5 mmol). The mixture was stirred at room temperature for 18 hours. It was loaded directly onto a silica gel column and eluted with a gradient of hexane and ethyl acetate to give 0.16 g (85%) of N-methyl-N-[2-({[5-(phenylsulfonyl)-2-(trifluoromethyl)phenyl]sulfonyl}amino)ethyl]pyrrolidine-1-carboxamide.

MS (ES+) m/z 519.7.

### EXAMPLE 834

### N-{2-[[(diethylamino)carbonyl](methyl)amino]ethyl}-5-(phenylsulfonyl)-2-(trifluoromethyl)benzenesulfonamide

In an analogous manner to Example 833, N-[2-(methylamino)ethyl]-5-(phenylsulfonyl)-2-(trifluoromethyl)benzenesulfonamide hydrochloride from Example 799 and diethylcarbamoyl chloride were used to prepare N-{2-[[(diethylamino)carbonyl](methyl)amino]ethyl}-5-(phenylsulfonyl)-2-(trifluoromethyl)benzenesulfonamide.

MS (ES+) m/z 521.8;

HRMS: calcd for C₂₁H₂₆F₃N₃O₅S₂+H+, 522.13387; found (ESI, [M+H]⁺), 522.1322.

### EXAMPLE 835

### N-methyl-N-[2-({[5-(phenylsulfonyl)-2-(trifluoromethyl)phenyl]sulfonyl}amino)ethyl]morpholine-4-carboxamide

In an analogous manner to Example 833, N-[2-(methylamino)ethyl]-5-(phenylsulfonyl)-2-(trifluoromethyl)benzenesulfonamide hydrochloride from Example 799 and 4-morpholinylcarbonyl chloride were used to prepare N-methyl-N-[2-({[5-(phenylsulfonyl)-2-(trifluoromethyl)phenyl]sulfonyl}amino)ethyl]morpholine-4-carboxamide.

MS (ES+) m/z 535.7;

HRMS: calcd for C₂₁H₂₄F₃N₃O₆S₂+H+, 536.11314; found (ESI, [M+H]⁺), 536.1124.

### EXAMPLE 836

### N-[2-(methyl{[methyl(phenyl)amino]carbonyl}amino)ethyl]-5-(phenylsulfonyl)-2-(trifluoromethyl)benzenesulfonamide

In an analogous manner to Example 833, N-[2-(methylamino)ethyl]-5-(phenylsulfonyl)-2-(trifluoromethyl)benzenesulfonamide hydrochloride from Example 799 and N-methyl-N-phenyl carbamoyl chloride were used to prepare N-[2-(methyl{[methyl(phenyl)amino]carbonyl}amino)ethyl]-5-(phenylsulfonyl)-2-(trifluoromethyl)benzenesulfonamide.

MS (ES+) m/z 555.7;

HRMS: calcd for C₂₄H₂₄F₃N₃O₅S₂+H+, 556.11822; found (ESI, [M+H]⁺), 556.1173.

### EXAMPLE 837

### N-Methyl-N-[2-({[5-(phenylsulfonyl)-2-(trifluoromethyl)phenyl]sulfonyl}amino)ethyl]-2-furamide

In an analogous manner as described in Example 819, and replacing benzoyl chloride with furan-2-carbonyl chloride, gave N-methyl-N-[2-({[5-(phenylsulfonyl)-2-(trifluoromethyl)phenyl]sulfonyl}amino)ethyl]-2-furamide (156.3 mg, 93%) as a white solid.

MS (ES+) m/z 516.6;

HRMS: calcd for C₂₁H₁₉F₃N₂O₆S₂+H⁺, 517.07094; found (ESI, [M+H]⁺), 517.072;

Anal. Calcd for C₂₁H₁₉F₃N₂O₆S₂: C, 48.83; H, 3.71; N, 5.42. Found: C, 49.02; H, 3.44; N, 5.31.

### EXAMPLE 838

### 4-tert-Butyl-N-methyl-N-[2-({[5-(phenylsulfonyl)-2-(trifluoromethyl)phenyl]sulfonyl}amino)ethyl]benzamide

In an analogous manner as described in Example 819, and replacing benzoyl chloride with 4-tert-butyl-benzoyl chloride, gave 4-tert-butyl-N-methyl-N-[2-({[5-(phenylsulfonyl)-2-(trifluoromethyl)phenyl]sulfonyl}amino)ethyl]benzamide (184.0 mg, 97%) as a white solid.

MS (ESI+) m/z 583;

HRMS: calcd for C₂₇H₂₉F₃N₂O₅S₂+H⁺, 583.15427; found (ESI, [M+H]⁺), 583.1552;

Anal. Calcd for C₂₇H₂₉F₃N₂O₅S₂: C, 55.66; H, 5.02; N, 4.81. Found: C, 55.63; H, 4.90; N, 4.54.

### EXAMPLE 839

### N-Methyl-N-[2-({[5-(phenylsulfonyl)-2-(trifluoromethyl)phenyl]sulfonyl}amino)ethyl]-2-(trifluoromethoxy)benzamide

In an analogous manner as described in Example 819, and replacing benzoyl chloride with 2-trifluoromethoxy-benzoyl chloride, gave N-methyl-N-[2-({[5-(phenylsulfonyl)-2-(trifluoromethyl)phenyl]sulfonyl}amino)ethyl]-2-(trifluoromethoxy)benzamide (184.1 mg, 92%) as a white solid foam.

MS (ESI+) m/z 611;

HRMS: calcd for C₂₄H₂₀F₆N₂O₆S₂+H⁺, 611.07397; found (ESI, [M+H]⁺), 611.0728;

Anal. Calcd for C₂₄H₂₀F₆N₂O₆S₂: C, 47.21; H, 3.30; N, 4.59. Found: C, 47.51; H, 2.90; N, 4.43.

### EXAMPLE 840

### 5-(phenylsulfonyl)-N-pyrrolidin-3-yl-2-(trifluoromethyl)benzenesulfonamide

Step 1: Following the same procedure described on example 435, 2-trifluoromethyl-5-(phenylsulfonyl)-benzenesulfonyl chloride and 3-aminopyrrolidine-1-carboxylic acid tert-butyl ester were used to prepare tert-butyl-3-({[5-(phenylsulfonyl)-2-(trifluoromethyl)phenyl]sulfonyl}amino)pyrrolidine-1-carboxylate.

Step 2: Following the same procedure described on example 688, tert-butyl-3-({[5-(phenylsulfonyl)-2-(trifluoromethyl)phenyl]sulfonyl}amino)pyrrolidine-1-carboxylate was used to prepare 5-(phenylsulfonyl)-N-pyrrolidin-3-yl-2-(trifluoromethyl)benzenesulfonamide.

MS (ESI+) m/z 435;

HPLC purity 94.2% at 210-370 nm, 6.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₇H₁₇F₃N₂O₄S₂+H+, 435.06546; found (ESI, [M+H]⁺), 435.0666.

### EXAMPLE 841

### N-[(2R*,4S*,6S*)-2,6-dimethyltetrahydro-2H-pyran-4-yl]-5-[(4-fluorophenyl)sulfonyl]-2-methylbenzenesulfonamide

Step 1: 2,6-Dimethyl-gamma-pyrone (5.0 g, 40.28 mmol) was hydrogenated with palladium on carbon (250 mg) in ethanol (50 mL) at 50 psi. The reaction was filtered through Celite® and washed with ethanol. The filtrate was concentrated to give (2R,6S)-2,6-dimethyldihydro-2H-pyran-4(3H)-one (5.1 g, 99%).

Step 2: To a solution of (2R,6S)-2,6-dimethyldihydro-2H-pyran-4(3H)-one (5.1 g, 39.79 mmol) in dichloromethane (50 mL) and 2,4-dimethoxybenzylamine (6.7 g, 39.79 mmol) was added sodium triacetoxyborohydride (11.8g, 55.70 mmol). The reaction was stirred overnight at room temperature under nitrogen. Added saturated bicarbonate solution and extracted. Dried with magnesium sulfate and concentrated to give (2R,4S,6S)-N-(2,4-dimethoxybenzyl)-2,6-dimethyltetrahydro-2H-pyran-4-amine (10.8 g, 97%).

Step 3: To a solution of 5-(4-fluorophenylsulfonyl)-2-methylbenzene-1-sulfonyl chloride (150 mg, 0.43 mmol) in dichloromethane (5.0 mL) was added triethylamine (137 μL, 0.25 mmol) followed by (2R,4S,6S)-N-(2,4-dimethoxybenzyl)-2,6-dimethyltetrahydro-2H-pyran-4-amine (132 mg, 0.47 mmol). The reaction was stirred overnight at room temperature. The following day the reaction was concentrated down onto silica gel and purified using automated chromatography with a gradient mobile phase consisting of ethyl acetate and hexane resulting in the isolation of N-(2,4-dimethoxybenzyl)-N-((2R,4s,6S)-2,6-dimethyltetrahydro-2H-pyran-4-yl)-5-(4-fluorophenylsulfonyl)-2-methylbenzenesulfonamide (96 mg, 38%).

Step 4: N-(2,4-dimethoxybenzyl)-N-((2R,4S,6S)-2,6-dimethyltetrahydro-2H-pyran-4-yl)-5-(4-fluorophenylsulfonyl)-2-methylbenzenesulfonamide (96 mg, 0.16 mmol) was dissolved in 6% trifluoroacetic acid/dichloromethane (6 mL) and stirred overnight at room temperature under nitrogen. Saturated bicarbonate solution was added and the reaction was extracted. Dried with magnesium sulfate and concentrated onto silica gel and purified using automated chromatography with a gradient mobile phase consisting of ethyl acetate and hexane resulting in the isolation of N-[(2R*,4S*,6S*)-2,6-dimethyltetrahydro-2H-pyran-4-yl]-5-[(4-fluorophenyl)sulfonyl]-2-methylbenzenesulfonamide (33 mg, 46%).

MS (ES−) m/z 440;

HRMS: calcd for C₂₀H₂₄FNO₅S₂+H+, 442.115821; found (ESI, [M+H]⁺), 442.1147.

### EXAMPLE 842

### 5-{[4-(methylamino)phenyl]sulfonyl}-N-piperidin-4-yl-2-(trifluoromethyl)benzenesulfonamide

Step 1: A stirred solution of tert-butyl 4-({[5-[(4-fluorophenyl)sulfonyl]-2-(trifluoromethyl)phenyl]sulfonyl}amino)piperidine-1-carboxylate (0.10 g, 0.18 mmol) in 33% methylamine in ethanol (0.5 mL) was microwave irradiated for 15 minutes at 160° C. The resulting solution was washed with ammonium chloride solution (sat.) and extracted with ethyl acetate. The organic layer was concentrated to give tert-butyl 4-({[5-[(4-methylaminophenyl)sulfonyl]-2-(trifluoromethyl)phenyl]sulfonyl}amino)piperidine-1-carboxylate.

Step 2: Following the same procedure described on example 688, tert-butyl 4-({[5-[(4-methylaminophenyl)sulfonyl]-2-(trifluoromethyl)phenyl]sulfonyl}amino)piperidine-1-carboxylate was used to prepare 5-{[4-(methylamino)phenyl]sulfonyl}-N-piperidin-4-yl-2-(trifluoromethyl)benzenesulfonamide.

MS (ESI+) m/z 478;

HPLC purity 100% at 210-370 nm, 7.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₉H₂₂F₃N₃O₄S₂+H+, 478.10766; found (ESI, [M+H]⁺), 478.1064.

### EXAMPLE 843

### 5-{[4-(dimethylamino)phenyl]sulfonyl}-N-piperidin-4-yl-2-(trifluoromethyl)benzenesulfonamide

Step 1: Following the same procedure described on example 842, dimethylamine in THF and tert-butyl 4-({[5-[(4-fluorophenyl)sulfonyl]-2-(trifluoromethyl)phenyl]sulfonyl}amino)piperidine-1-carboxylate was used to prepare tert-butyl 4-({[5-[(4-dimethylaminophenyl)sulfonyl]-2-(trifluoromethyl)phenyl]sulfonyl}amino)piperidine-1-carboxylate.

Step 2: Following the same procedure described on example 688, tert-butyl 4-({[5-[(4-dimethylaminophenyl)sulfonyl]-2-(trifluoromethyl)phenyl]sulfonyl}amino)piperidine-1-carboxylate was used to prepare 5-{[4-(dimethylamino)phenyl]sulfonyl}-N-piperidin-4-yl-2-(trifluoromethyl)benzenesulfonamide.

MS (ESI+) m/z 492;

HPLC purity 97.1% at 210-370 nm, 7.5 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₀H₂₄F₃N₃O₄S₂+H+, 492.12331; found ESI, [M+H]⁺), 492.1243.

### EXAMPLE 844

### 5-({4-[(2-hydroxyethyl)amino]phenyl}sulfonyl)-N-piperidin-4-yl-2-(trifluoromethyl)benzenesulfonamide

Step 1: Following the same procedure described on example 842, ethanolamine in THF and tert-butyl 4-({[5-[(4-fluorophenyl)sulfonyl]-2-(trifluoromethyl)phenyl]sulfonyl}amino)piperidine-1-carboxylate was used to prepare 4-({[5-[(4-[(2-hydroxyethyl)amino]phenyl)sulfonyl]-2-(trifluoromethyl)phenyl]sulfonyl}amino)piperidine-1-carboxylate.

Step 2: Following the same procedure described on example 688, 4-({[5-[(4-[(2-hydroxyethyl)amino]phenyl)sulfonyl]-2-(trifluoromethyl)phenyl]sulfonyl}amino)piperidine-1-carboxylate was used to prepare 5-({4-[(2-hydroxyethyl)amino]phenyl}sulfonyl)-N-piperidin-4-yl-2-(trifluoromethyl)benzenesulfonamide.

MS (ESI+) m/z 508;

HPLC purity 95.1% at 210-370 nm, 6.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₀H₂₄F₃N₃O₅S₂+H+, 508.11822; found (ESI, [M+H]⁺), 508.1198.

### EXAMPLE 845

### 5-[(2-fluorophenyl)sulfonyl]-N-[2-(1H-imidazol-1-yl)ethyl]-2-(trifluoromethyl)benzenesulfonamide

To a mixture of sodium carbonate (0.1601 g, 1.5 mmol) in 1 ml of water and 2-(1H-imidazol-1-yl)ethyl amine (0.092, 0.5 mmol) in 3 ml of acetonitrile was stirred at R.T. as the mixture of 5-(4-fluorophenylsulfonyl)-2-(trifluoromethyl)benzene-1-sulfonyl chloride and 5-(2-fluorophenylsulfonyl)-2-(trifluoromethyl)benzene-1-sulfonyl chloride (0.2514 g, 0.6 mmol) prepared in Example 816 Step 4 was taken up in 10 ml of acetonitrile was slowly added to the reaction with an addition funnel. The reaction was allowed to stir overnight at room temperature and then was partitioned between ethyl acetate and 2N hydrochloric acid. The ethyl acetate layer was separated, dried (anhydrous MgSO₄) and the solvent removed under reduced pressure to give approximately 50 mg of the isomeric mixture that was dissolved in 3 mL of methanol/acetonitrile. 200 μL of the resulting solution was repetitively injected onto the Supercritical Fluid Chromatography (SFC) instrument, and the baseline resolved isomers were separately collected using the conditions described below to give the title compound 5-[(2-fluorophenyl)sulfonyl]-N-[2-(1H-imidazol-1-yl)ethyl]-2-(trifluoromethyl)benzenesulfonamide (19.8 mg, 7%) as a white solid.

MS (ESI+) m/z 478;

HPLC purity 97.8% at 210-370 nm, 6.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₈H₁₅F₄N₃O₄S₂+H+, 478.05129; found (ESI, [M+H]+), 478.0506.

### EXAMPLE 846

### N-methyl-N-[2-({[5-(phenylsulfonyl)-2-(trifluoromethyl)phenyl]sulfonyl}amino)ethyl]cyclohexanecarboxamide

A stirred mixture of tert-butyl methyl[2-({[5-(phenylsulfonyl)-2-(trifluoromethyl)phenyl]sulfonyl}amino)ethyl]carbamate (0.1062 g, 0.25 mmol) prepared in the same manner as Example 799 in dichloromethane (2 ml). 1.1 eq of cyclohexanecarbonyl chloride (35 μL, 0.26 mmol) and 1.5 eq. N,N-Diisoprpylethylamine was syringed into the reaction vial and was allowed to stir overnight at room temperature. The product was transferred onto a 4 g Isco RediSep® Normal Phase column and was purified by automated flash chromatography using a gradient of 20% to 100% hexane/ethyl acetate. Isolation of the main component gave the title compound N-methyl-N-[2-({[5-(phenylsulfonyl)-2-(trifluoromethyl)phenyl]sulfonyl}amino)ethyl]cyclohexanecarboxamide (58.3 mg, 44%) as a white solid.

MS (ESI+) m/z 533;

HPLC purity 100% at 210-370 nm, 9.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₃H₂₇F₃N₂O₅S₂+H+, 533.13862; found (ESI, [M+H]+), 533.138.

### EXAMPLE 847

### 3-Fluoro-N-methyl-N-[2-({[5-(phenylsulfonyl)-2-(trifluoromethyl)phenyl]sulfonyl}amino)ethyl]-4-(trifluoromethyl)benzamide

In an analogous manner as described in Example 819, and replacing benzoyl chloride with 3-fluoro-4-trifluoromethyl-benzoyl chloride, gave 3-fluoro-N-methyl-N-[2-({[5-(phenylsulfonyl)-2-(trifluoromethyl)phenyl]sulfonyl}amino)ethyl]-4-(trifluoromethyl)benzamide (168.1 mg, 84%) as a white solid.

MS (ESI+) m/z 613;

HRMS: calcd for C₂₄H₁₉F₇N₂O₅S₂+H⁺, 613.06964; found (ESI, [M+H]⁺), 613.0691;

Anal. Calcd for C₂₄H₁₉F₇N₂O₅S₂: C, 47.06; H, 3.13; N, 4.57. Found: C, 46.83; H, 3.00; N, 4.34.

### EXAMPLE 848

### Methyl 4-({methyl[2-({[5-(phenylsulfonyl)-2-(trifluoromethyl)phenyl]sulfonyl}amino)ethyl]amino}carbonyl)benzoate

In an analogous manner as described in Example 819, and replacing benzoyl chloride with 4-chlorocarbonyl-benzoic acid methyl ester, gave methyl 4-({methyl[2-({[5-(phenylsulfonyl)-2-(trifluoromethyl)phenyl]sulfonyl}amino)ethyl]amino}carbonyl)benzoate (155.8 mg, 81%) as a white solid.

MS (ESI+) m/z 585;

HRMS: calcd for C₂₅H₂₃F₃N₂O₇S₂+H⁺, 585.09715; found (ESI, [M+H]⁺), 585.0983;

Anal. Calcd for C₂₅H₂₃F₃N₂O₇S₂: C, 51.37; H, 3.97; N, 4.79. Found: C, 51.13; H, 3.74; N, 4.63.

### EXAMPLE 849

### N-Methyl-N-[2-({[5-(phenylsulfonyl)-2-(trifluoromethyl)phenyl]sulfonyl}amino)ethyl]nicotinamide

In an analogous manner as described in Example 819, and replacing benzoyl chloride with nicotinoyl chloride, extracting with water instead of 2 N HCl, and using 100% methylene chloride to 5% methanol-methylene chloride as the eluent, gave N-methyl-N-[2-({[5-(phenylsulfonyl)-2-(trifluoromethyl)phenyl]sulfonyl}amino)ethyl]nicotinamide (166.6 mg, 97%) as a white solid foam.

MS (ESI+) m/z 528;

HRMS: calcd for C₂₂H₂₀F₃N₃O₅S₂+H⁺, 528.08692; found (ESI, [M+H]⁺), 528.0885;

Anal. Calcd for C₂₂H₂₀F₃N₃O₅S₂: C, 50.09; H, 3.82; N, 7.97. Found: C, 49.99; H, 3.59; N, 7.72.

### EXAMPLE 850

### N-Methyl-N-[2-({[5-(phenylsulfonyl)-2-(trifluoromethyl)phenyl]sulfonyl}amino)ethyl]isonicotinamide

In an analogous manner as described in Example 849, and replacing nicotinoyl chloride with isonicotinoyl chloride gave N-methyl-N-[2-({[5-(phenylsulfonyl)-2-(trifluoromethyl)phenyl]sulfonyl}amino)ethyl]isonicotinamide (151.1 mg, 88%) as a white solid.

MS (ESI+) m/z 528;

HRMS: calcd for C₂₂H₂₀F₃N₃O₅S₂+H⁺, 528.08692; found (ESI, [M+H]⁺), 528.0889;

Anal. Calcd for C₂₂H₂₀F₃N₃O₅S₂.0.70 CH₂Cl₂: C, 46.45; H, 3.67; N, 7.16. Found: C, 46.71; H, 3.46; N, 7.18.

### EXAMPLE 851

### 2-Chloro-N-methyl-N-[2-({[5-(phenylsulfonyl)-2-(trifluoromethyl)phenyl]sulfonyl}amino)ethyl]nicotinamide

In an analogous manner as described in Example 849, and replacing nicotinoyl chloride with 2-chloro-nicotinoyl chloride gave 2-chloro-N-methyl-N-[2-({[5-(phenylsulfonyl)-2-(trifluoromethyl)phenyl]sulfonyl}amino)ethyl]nicotinamide (168.4 mg, 91%) as a white solid foam.

MS (ESI+) m/z 562;

HRMS: calcd for C₂₂H₁₉ClF₃N₃O₅S₂+H⁺, 562.04795; found (ESI, [M+H]⁺), 562.0471;

Anal. Calcd for C₂₂H₁₉ClF₃N₃O₅S₂.0.40 CH₂Cl₂: C, 45.15; H, 3.35; N, 7.05. Found: C, 46.48; H, 3.19; N; 7.32.

### EXAMPLE 852

### N,2,2-trimethyl-N-[2-({[5-(phenylsulfonyl)-2-(trifluoromethyl)phenyl]sulfonyl}amino)ethyl]propanamide

In an analogous manner to example 846, tert-butyl methyl[2-({[5-(phenylsulfonyl)-2-(trifluoromethyl)phenyl]sulfonyl}amino)ethyl]carbamate and trimethylacetyl chloride was used to prepare the title compound N,2,2-trimethyl-N-[2-({[5-(phenylsulfonyl)-2-(trifluoromethyl)phenyl]sulfonyl}amino)ethyl]propanamide (102 mg, 81%) as a white solid.

MS (ES+) m/z 506.7;

HPLC purity 98.6% at 210-370 nm, 9.5 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₁H₂₅F₃N₂O₅S₂+H+, 507.12297; found (ESI, [M+H]+), 507.1238.

### EXAMPLE 853

### 3-{[4-({[5-(phenylsulfonyl)-2-(trifluoromethyl)phenyl]sulfonyl}amino)piperidin-1-yl]sulfonyl}benzoic acid

In an analogous manner to example 462, 5-(phenylsulfonyl)-N-piperidin-4-yl-2-(trifluoromethyl)benzenesulfonamide and 3-(chlorosulfonyl)benzoic acid were used to prepare 3-{[4-({[5-(phenylsulfonyl)-2-(trifluoromethyl)phenyl]sulfonyl}amino)piperidin-1-yl]sulfonyl}benzoic acid.

HPLC purity 97.1% at 210-370 nm, 9.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₅H₂₃F₃N₂O₈S₃+H+, 633.06414; found (ESI, [M+H]⁺), 633.0627.

### EXAMPLE 854

### 4-{[4-({[5-(phenylsulfonyl)-2-(trifluoromethyl)phenyl]sulfonyl}amino)piperidin-1-yl]sulfonyl}benzoic acid

In an analogous manner to example 462, 5-(phenylsulfonyl)-N-piperidin-4-yl-2-(trifluoromethyl)benzenesulfonamide and 4-(chlorosulfonyl)benzoic acid were used to prepare 4-{[4-({[5-(phenylsulfonyl)-2-(trifluoromethyl)phenyl]sulfonyl}amino)piperidin-1-yl]sulfonyl}benzoic acid.

MS (ES+) m/z 632.5;

HPLC purity 100% at 210-370 nm, 9.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 855

### 3-({[4-({[5-(phenylsulfonyl)-2-(trifluoromethyl)phenyl]sulfonyl}amino)piperidin-1-yl]carbonothioyl}amino)benzoic acid

In an analogous manner to example 462, 5-(phenylsulfonyl)-N-piperidin-4-yl-2-(trifluoromethyl)benzenesulfonamide and 3-carboxyphenyl isothiocyanate were used to prepare 3-({[4-({[5-(phenylsulfonyl)-2-(trifluoromethyl)phenyl]sulfonyl}amino)piperidin-1-yl]carbonothioyl}amino)benzoic acid.

MS (ES+) m/z 627.5;

HPLC purity 98.4% at 210-370 nm, 9.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₆H₂₄F₃N₃O₆S₃+H+, 628.08521; found (ESI, [M+H]⁺), 628.0853.

### EXAMPLE 856

### N-[1-(2-chloro-6-methylisonicotinoyl)piperidin-4-yl]-5-(phenylsulfonyl)-2-(trifluoromethyl)benzenesulfonamide

In an analogous manner to example 462, 5-(phenylsulfonyl)-N-piperidin-4-yl-2-(trifluoromethyl)benzenesulfonamide and 2-chloro-6-methylpyridine-4-carbonyl chloride were used to prepare N-[1-(2-chloro-6-methylisonicotinoyl)piperidin-4-yl]-5-(phenylsulfonyl)-2-(trifluoromethyl)benzenesulfonamide.

MS (ES−) m/z 599.5;

HPLC purity 95.5% at 210-370 nm, 9.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₅H₂₃ClF₃N₃O₅S₂+H+, 602.07925; found (ESI, [M+H]⁺), 602.0812.

### EXAMPLE 857

### N-[1-(2,6-dichloroisonicotinoyl)piperidin-4-yl]-5-(phenylsulfonyl)-2-(trifluoromethyl)benzenesulfonamide

In an analogous manner to example 462, 5-(phenylsulfonyl)-N-piperidin-4-yl-2-(trifluoromethyl)benzenesulfonamide and 2,6-dichloropyridine-4-carbonyl chloride were used to prepare N-[1-(2,6-dichloroisonicotinoyl)piperidin-4-yl]-5-(phenylsulfonyl)-2-(trifluoromethyl)benzenesulfonamide.

MS (ES+) m/z 621.5;

HPLC purity 91.5% at 210-370 nm, 9.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₄H₂₀Cl₂F₃N₃O₅S₂+H+, 622.02463; found (ESI, [M+H]⁺) 622.0237.

### EXAMPLE 858

### N-(1,1-dioxidotetrahydro-2H-thiopyran-4-yl)-5-[(4-fluorophenyl)sulfonyl]-2-isopropylbenzenesulfonamide

Step 1: To a solution of tetrahydrothiopyran (3.0 g, 25.82 mmol) and 2,4-dimethoxybenzylamine (4.3 g, 25.82 mmol) in dichloromethane (100 mL) was added sodium triacetoxyborohydride (7.7 g, 36.15 mmol). The reaction was stirred overnight at room temperature under nitrogen. Added saturated bicarbonate solution and extracted. Dried with magnesium sulfate and concentrated to give N-(2,4-dimethoxybenzyl)tetrahydro-2H-thiopyran-4-amine (6.7 g, 97%).

Step 2: To a solution of 5-(4-fluorophenylsulfonyl)-2-isopropylbenzene-1-sulfonyl chloride (350 mg, 0.93 mmol) in dichloromethane 10 mL) was added triethylamine (388 μL, 2.79 mmol) followed by N-(2,4-dimethoxybenzyl)tetrahydro-2H-thiopyran-4-amine (497 mg, 1.86 mmol). The reaction was stirred overnight at room temperature. The following day the reaction was concentrated down onto silica gel and purified using automated chromatography with a gradient mobile phase consisting of ethyl acetate and hexane resulting in the isolation of N-(2,4-dimethoxybenzyl)-5-(4-fluorophenylsulfonyl)-2-isopropyl-N-(tetrahydro-2H-thiopyran-4-yl)benzenesulfonamide (504 mg, 89%).

Step 3: N-(2,4-dimethoxybenzyl)-5-(4-fluorophenylsulfonyl)-2-isopropyl-N-(tetrahydro-2H-thiopyran-4-yl)benzenesulfonamide (504 mg, 0.83 mmol) was dissolved in 6% trifluoroacetic acid/dichloromethane (6 mL) and stirred overnight at room temperature under nitrogen. Saturated bicarbonate solution was added and the reaction was extracted. Dried with magnesium sulfate and concentrated onto silica gel and purified using automated chromatography with a gradient mobile phase consisting of ethyl acetate and hexane resulting in the isolation of 5-[(4-fluorophenyl)sulfonyl]-2-isopropyl-N-(tetrahydro-2H-thiopyran-4-yl)benzenesulfonamide (350 mg, 92%).

Step 4: 5-[(4-fluorophenyl)sulfonyl]-2-isopropyl-N-(tetrahydro-2H-thiopyran-4-yl)benzenesulfonamide (150 mg, 0.32 mmol) was dissolved in dichloromethane (8 mL) and 3-chloroperoxybenzoic acid (154 mg, 0.69 mmol) was added. The reaction was stirred overnight at room temperature and was then concentrated onto silica gel and purified using automated chromatography with a gradient mobile phase consisting of ethyl acetate and hexane resulting in the isolation of N-(1,1-dioxidotetrahydro-2H-thiopyran-4-yl)-5-[(4-fluorophenyl)sulfonyl]-2-isopropylbenzenesulfonamide (80 mg, 50%).

MS (ES−) m/z 487.6.

### EXAMPLE 859

### 5-(Phenylsulfonyl)-2-propyl-N-(2-pyridin-4-ylethyl)benzenesulfonamide

The title compound was prepared from 5-phenylsulfonyl-2-propyl-benzenesulfonyl chloride (0.36 g, 1.0 mmol) and 2-(pyridin-4-yl)ethanamine (0.25 g, 2.0 mmol) according to the procedure and in the same manner as described in Example 550, step c. The crude product was purified by preparative liquid chromatography on a Biotage® 40 Si column of pre-packed silica gel (90 g), eluting with a gradient of 50%-100% methyl tert-butyl ether in hexane at a flow rate of 50 mL/min, to afford 5-(phenylsulfonyl)-2-propyl-N-[2-(tetrahydro-2H-pyran-4-yl)ethyl]benzenesulfonamide (0.26 g, 58%), as a homogeneous, amorphous solid, m.p. 155-157° C.;

MS (+ESI), m/z: 444.7 [M+H]⁺;

HRMS: calcd for C₂₂H₂₄N₂O₄S₂+H+, 445.12502; found (ESI, [M+H]⁺), 445.1229;

HPLC purity 100% at 210-370 nm, 8.5 min; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 860

### 2-Isopropyl-N-[(1R*,5S*)-8-methyl-8-azabicyclo[3.2.1]oct-3-yl]-5-(phenylsulfonyl)benzenesulfonamide monohydrochloride

A stirred solution of 2-isopropyl-5-(phenylsulfonyl)benzenesulfonyl chloride (0.36 g, 1.0 mmol) in 3:1 dichloromethane-acetonitrile (10 mL) was treated under nitrogen with endo-8-methyl-8-azabicyclo[3.2.1]octan-3-amine dihydrochloride (0.42 g, 2.0 mmol) and diisopropylethylamine (0.52 g, 4.0 mmol). The reaction was stirred for 18 hours at room temperature. The crude product was purified by reverse phase preparative liquid chromatography on a Gilson® semi-prep column, eluting with a gradient of 10%-100% water in acetonitrile at a flow rate of 2 mL/min, to afford, after concentration of the solvent and extraction with ethyl acetate (3×), an oil. The oil was dissolved in ethanol-dioxane and treated with a solution of 1N hydrogen chloride in diethyl ether. Filtration of the solid afforded 2-isopropyl-N-[(1R*,5S*)-8-methyl-8-azabicyclo[3.2.1]oct-3-yl]-5-(phenylsulfonyl)benzenesulfonamide monohydrochloride (0.10 g, 20%), as a homogeneous, cream-colored, amorphous solid, m.p. 90° C.

MS (+ESI), m/z: 462.8 [M+H]⁺;

HRMS: calcd for C₂₃H₃₀N₂O₄S₂+H+, 463.17197; found (ESI, [M+H]⁺), 463.171;

HPLC purity 98.7% at 210-370 nm, 9.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 861

### 5-{[2-(methylamino)phenyl]sulfonyl}-N-piperidin-4-yl-2-(trifluoromethyl)benzenesulfonamide

Step 1: Following the same procedure described on example 677 Step 1, 4-chloro-3-nitrobenzenesulfonyl chloride and fluorobenzene was used to prepare 5-(2-fluorophenylsulfonyl)-2-chloro-nitrobenzene.

Step 2: Following the same procedure described on example 677 Step 2, 5-(2-fluorophenylsulfonyl)-2-chloro-nitrobenzene was used to prepare 5-(2-fluorophenylsulfonyl)-2-trifluoromethyl-nitrobenzene.

Step 3: Following the same procedure described on example 677 Step 3, 5-(2-fluorophenylsulfonyl)-2-trifluoromethyl-nitrobenzene was used to prepare 5-(2-fluorophenylsulfonyl)-2-trifluoromethylaniline.

Step 4: Following the same procedure described on example 677 Step 4, 5-(2-fluorophenylsulfonyl)-2-trifluoromethylaniline was used to prepare 2-trifluoromethyl-5-(2-fluorophenylsulfonyl)-benzenesulfonyl chloride.

Step 5: Following the same procedure described on example 435, 2-trifluoromethyl-5-(2-fluorophenylsulfonyl)- benzenesulfonyl chloride and 4-aminopiperidine-1-carboxylic acid tert-butyl ester were used to prepare tert-butyl 4-({[5-(2-fluorophenylsulfonyl)-2-(trifluoromethyl)phenyl]sulfonyl}amino)piperidine-1-carboxylate.

Step 6: Following the same procedure described on example 842 Step 1, tert-butyl 4-({[5-(2-fluorophenylsulfonyl)-2-(trifluoromethyl)phenyl]sulfonyl}amino)piperidine-1-carboxylate was used to prepare tert-butyl 4-({[5-(2-methylaminophenylsulfonyl)-2-(trifluoromethyl)phenyl]sulfonyl}amino)piperidine-1-carboxylate.

Step 7: Following the same procedure described on example 842 Step 2: tert-butyl 4-({[5-(2-methylaminophenylsulfonyl)-2-(trifluoromethyl)phenyl]sulfonyl}amino)piperidine-1-carboxylate was used to prepare 5-{[2-(methylamino)phenyl]sulfonyl}-N-piperidin-4-yl-2-(trifluoromethyl)benzenesulfonamide.

MS (ES+) m/z 477.7;

HPLC purity 100% at 210-370 mn, 7.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₉H₂₂F₃N₃O₄S₂+H+, 478.10766; found (ESI, [M+H]⁺), 478.1078.

### EXAMPLE 862

### 5-{[2-(dimethylamino)phenyl]sulfonyl}-N-piperidin-4-yl-2-(trifluoromethyl)benzenesulfonamide

Step 1: Following the same procedure described on example 842 Step 1, tert-butyl 4-({[5-(2-fluorophenylsulfonyl)-2-(trifluoromethyl)phenyl]sulfonyl}amino)piperidine-1-carboxylate and dimethylamine were used to prepare tert-butyl 4-({[5-(2-dimethylaminophenylsulfonyl)-2-(trifluoromethyl)phenyl]sulfonyl}amino)piperidine-1-carboxylate.

Step 2: Following the same procedure described on example 842 Step 2, tert-butyl 4-({[5-(2-dimethylaminophenylsulfonyl)-2-(trifluoromethyl)phenyl]sulfonyl}amino)piperidine-1-carboxylate was used to prepare 5-{[2-(dimethylamino)phenyl]sulfonyl}-N-piperidin-4-yl-2-(trifluoromethyl)benzenesulfonamide.

MS (ES+) m/z 491.7;

HPLC purity 95.8% at 210-370 nm, 7.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 863

### 5-(phenylsulfonyl)-N-[2-(2H-tetrazol-5-yl)ethyl]-2-(trifluoromethyl)benzenesulfonamide

To a stirred solution of trimethylaluminum 2M in toluene (0.13 mL, 0.26 mmol) and trimethylsilylazide (0.03 g, 0.26 mmol) in toluene (1 mL) at 0° C. was added N-(2-cyanoethyl)-5-(phenylsulfonyl)-2-(trifluoromethyl)benzenesulfonamide (0.09 g, 0.22 mmol). The resulting solution was heated to 80° C. overnight, cooled to 0° C., quenched with 6N HCl solution and extracted with ethyl acetate. The organic layer was concentrated and the resulting solid was triturated in ethyl acetate to give 5-(phenylsulfonyl)-N-[2-(2H-tetrazol-5-yl)ethyl]-2-(trifluoromethyl)benzenesulfonamide. (0.045 g, 45%).

MS (ES+) m/z 461.6;

HPLC purity 100% at 210-370 nm, 7.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₆H₁₄F₃N₅O₄S₂+H+, 462.05121; found (ESI, [M+H]⁺), 462.049.

### EXAMPLE 864

### 2-ethyl-N-methyl-N-[2-({[5-(phenylsulfonyl)-2-(trifluoromethyl)phenyl]sulfonyl}amino)ethyl]butanamide

In an analogous manner to example 846, tert-butyl methyl[2-({[5-(phenylsulfonyl)-2-(trifluoromethyl)phenyl]sulfonyl}amino)ethyl]carbamate and 2-Ethylbutyryl chloride was used to prepare the title compound 2-ethyl-N-methyl-N-[2-({[5-(phenylsulfonyl)-2-(trifluoromethyl)phenyl]sulfonyl}amino)ethyl]butanamide (47.8 mg, 36.1%) as a white solid.

MS (ES+) m/z 520.6;

HPLC purity 100% at 210-370 nm, 9.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₂H₂₇F₃N₂O₅S₂+H+, 521.13862; found (ESI, [M+H]⁺), 521.1391.

### EXAMPLE 865

### butyl methyl[2-({[5-(phenylsulfonyl)-2-(trifluoromethyl)phenyl]sulfonyl}amino)ethyl]carbamate

In an analogous manner to example 846, tert-butyl methyl[2-({[5-(phenylsulfonyl)-2-(trifluoromethyl)phenyl]sulfonyl}amino)ethyl]carbamate and butyl chloroformate was used to prepare the title compound butyl methyl[2-({[5-(phenylsulfonyl)-2-(trifluoromethyl)phenyl]sulfonyl}amino)ethyl]carbamate (39.7 mg, 32%) as a white solid.

MS (ES+) m/z 522.6;

HPLC purity 100% at 210-370 nm, 9.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₁H₂₅F₃N₂O₆S₂+H+, 523.11789; found (ESI, [M+H]+), 523.1178.

### EXAMPLE 866

### tert-butyl 4-[({methyl[2-({[5-(phenylsulfonyl)-2-(trifluoromethyl)phenyl]sulfonylamino)ethyl]amino}carbonyl)amino]piperidine-1-carboxylate

Step 1: To a mixture of N,N′-carbonyldiimidazole (143.0 mg, 0.8819 mmol) and 4-amino-1-BOC-piperidine (160.6 mg, 0.80 mmol) was added dichloromethane (10 mL). The reaction was stirred at room temperature under N₂ for 18 hours. The solution was washed with H₂O (3×) and dried over MgSO₄. The solvent was evaporated to give 224.7 mg (95%) of intermediate tert-butyl 4-[(1H-imidazol-1-ylcarbonyl)amino]piperidine-1-carboxylate.

Step 2: To a stirred suspension of N-[2-(methylamino)ethyl]-5-(phenylsulfonyl)-2-(trifluoromethyl)benzenesulfonamide hydrochloride (0.16 g, 0.35 mmol) from Example 799 in a solution of tert-butyl 4-[(1H-imidazol-1-ylcarbonyl)amino]piperidine-1-carboxylate (0.11 g, 0.37 mmol) in CH₂Cl₂ (5 mL) under N₂ at room temperature was added diisopropylethylamine (64 μL, 47 mg, 0.37 mmol). The mixture was stirred at room temperature for 3 days. The solution was washed 3 times with water. It was loaded directly onto a silica gel column and eluted with a gradient of hexane and ethyl acetate to give 0.18 g (80%) of tert-butyl 4-[({methyl[2-({[5-(phenylsulfonyl)-2-(trifluoromethyl)phenyl]sulfonyl}amino)ethyl]amino}carbonyl)amino]piperidine-1-carboxylate.

MS (ES−) m/z 646.7;

HRMS: calcd for C₂₇H₃₅F₃N₄O₇S₂+H+, 649.19720; found (ESI, [M+H]⁺), 649.1979.

### EXAMPLE 867

### 2,2-dimethylpropyl methyl[2-({[5-(phenylsulfonyl)-2-(trifluoromethyl)phenyl]sulfonyl}amino)ethyl]carbamate

In an analogous manner to example 846, tert-butyl methyl[2-({[5-(phenylsulfonyl)-2-(trifluoromethyl)phenyl]sulfonyl}amino)ethyl]carbamate and neopentyl chloroformate was used to prepare the title compound 2,2-dimethylpropyl methyl[2-({[5-(phenylsulfonyl)-2-(trifluoromethyl)phenyl]sulfonyl}amino)ethyl]carbamate (86.2 mg, 67%) as a white solid.

MS (ES+) m/z 536.8;

HPLC purity 99.0% at 210-370 nm, 9.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₂H₂₇F₃N₂O₆S₂+H+, 537.13354; found (ESI, [M+H]⁺), 537.1332.

### EXAMPLE 868

### N-[(1-hydroxycyclohexyl)methyl]-5-(phenylsulfonyl)-2-(trifluoromethyl)benzenesulfonamide

In an analogous manner to example 654, 2-trifluoromethyl-5-(phenylsulfonyl)-benzenesulfonyl chloride and 1-aminomethyl-1-cyclohexanol hydrochloride were used to prepare N-[(1-hydroxycyclohexyl)methyl]-5-(phenylsulfonyl)-2-(trifluoromethyl)benzenesulfonamide.

MS (ES) m/z 475.7;

HPLC purity 97.6% at 210-370 nm, 9.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 869

### 5-({2-[(2-hydroxyethyl)amino]phenyl}sulfonyl)-N-piperidin-4-yl-2-(trifluoromethyl)benzenesulfonamide

Step 1: Following the same procedure described on example 842 Step 1, tert-butyl 4-({[5-(2-fluorophenylsulfonyl)-2-(trifluoromethyl)phenyl]sulfonyl}amino)piperidine-1-carboxylate and ethanolamine were used to prepare tert-butyl 4-({[5-([2-hydroxyethyl)amino]phenylsulfonyl)-2-(trifluoromethyl)phenyl]sulfonyl}amino)piperidine-1-carboxylate.

Step 2: Following the same procedure described on example 842 Step 2, tert-butyl 4-({[5-([2-hydroxyethyl)amino]phenylsulfonyl)-2-(trifluoromethyl)phenyl]sulfonyl}amino)piperidine-1-carboxylate was used to prepare 5-({2-[(2-hydroxyethyl)amino]phenyl}sulfonyl)-N-piperidin-4-yl-2-(trifluoromethyl)benzenesulfonamide.

MS (ES) m/z 507.8;

HPLC purity 94.9% at 210-370 nm, 6.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min. HRMS: calcd for C₂₀H₂₄F₃N₃O₅S₂+H+, 508.11822; found (ESI, [M+H]⁺), 508.119.

### EXAMPLE 870

### isobutyl methyl[2-({[5-(phenylsulfonyl)-2-(trifluoromethyl)phenyl]sulfonyl}amino)ethyl]carbamate

In an analogous manner to example 846, tert-butyl methyl[2-({[5-(phenylsulfonyl)-2-(trifluoromethyl)phenyl]sulfonyl}amino)ethyl]carbamate and isobutyl chloroformate was used to prepare the title compound isobutyl methyl[2-({[5-(phenylsulfonyl)-2-(trifluoromethyl)phenyl]sulfonyl}amino)ethyl]carbamate (57.7 mg, 46%) as a white solid.

MS (ES+) m/z 522.9;

HPLC purity 99.1% at 210-370 nm, 9.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min. HRMS: calcd for C₂₁H₂₅F₃N₂O₆S₂+H+, 523.11789; found (ESI, [M+H]⁺), 523.1175.

### EXAMPLE 871

### 3-(trifluoromethyl)phenyl methyl[2-({[5-(phenylsulfonyl)-2-(trifluoromethyl)phenyl]sulfonyl}amino)ethyl]carbamate

In an analogous manner to example 846, tert-butyl methyl[2-({[5-(phenylsulfonyl)-2-(trifluoromethyl)phenyl]sulfonyl}amino)ethyl]carbamate and 3-(Trifluoromethyl)phenyl chloroformate was used to prepare the title compound 3-(trifluoromethyl)phenyl methyl[2-({[5-(phenylsulfonyl)-2-(trifluoromethyl)phenyl]sulfonyl}amino)ethyl]carbamate (77.8 mg, 53%) as a white solid.

MS (ES+) m/z 610.9;

HPLC purity 98.1% at 210-370 nm, 10.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₄H₂₀F₆N₂O₆S₂+H+, 611.07397; found (ESI, [M+H]⁺), 611.0731.

### EXAMPLE 872

### 4-fluorophenyl methyl[2-({[5-(phenylsulfonyl)-2-(trifluoromethyl)phenyl]sulfonyl]amino)ethyl]carbamate

In an analogous manner to example 846, tert-butyl methyl[2-({[5-(phenylsulfonyl)-2-(trifluoromethyl)phenyl]sulfonyl}amino)ethyl]carbamate and 4-Fluorophenyl chloroformate was used to prepare the title compound 4-fluorophenyl methyl[2-({[5-(phenylsulfonyl)-2-(trifluoromethyl)phenyl]sulfonyl}amino)ethyl]carbamate (85.6 mg, 62.4%) as a white solid.

MS (ES+) m/z 560.7;

HPLC purity 98.1% at 210-370 nm, 9.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₃H₂₀F₄N₂O₆S₂+H+, 561.07717; found (ESI, [M+H]⁺), 561.0757.

### EXAMPLE 873

### 4-bromophenyl methyl[2-({[5-(phenylsulfonyl)-2-(trifluoromethyl)phenyl]sulfonyl}amino)ethyl]carbamate

In an analogous manner to example 846, tert-butyl methyl[2-({[5-(phenylsulfonyl)-2-(trifluoromethyl)phenyl]sulfonyl}amino)ethyl]carbamate and 4-Bromophenyl chloroformate was used to prepare the title compound 4-bromophenyl methyl[2-({[5-(phenylsulfonyl)-2-(trifluoromethyl)phenyl]sulfonyl}amino)ethyl]carbamate (66.7mg, 39%) as a white solid.

MS (ES+) m/z 620.6;

HPLC purity 97.0% at 210-370 nm, 10.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₃H₂₀BrF₃N₂O₆S₂+H+, 620.99710; found (ESI, [M+H]⁺), 620.9971.

### EXAMPLE 874

### 5-[(4-Fluorophenyl)sulfonyl]-2-propyl-N-(2-pyridin-3-ylethyl)benzenesulfonamide

The title compound was prepared from 5-[(4-fluorophenyl)sulfonyl]-2-propylbenzenesulfonyl chloride (0.38 g, 1.0 mmol) and 2-(pyridin-3-yl)ethanamine (0.25 g, 0.2.0 mmol) according to the procedure and in the same manner as described in Example 559, step c. The crude product was purified by preparative liquid chromatography on a Biotage® 40 Si column of pre-packed silica gel (90 g), eluting with a gradient of 50%-100% methyl tert-butyl ether in hexane at a flow rate of 50 mL/min, to afford, after crystallization from ethyl acetate-diethyl ether-hexane, 5-[(4-fluorophenyl)sulfonyl]-2-propyl-N-(2-pyridin-3-ylethyl)benzenesulfonamide (0.27 g, 59%), as a homogeneous, colorless, crystalline solid, m.p. 111-113° C.;

MS (+ESI), m/z: 462.8 [M+H]⁺;

HRMS: calcd for C₂₂H₂₃FN₂O₄S₂+H+, 463.11560; found (ESI, [M+H]⁺), 463.1161;

HPLC purity 100% at 210-370 nm, 8.9 min; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 875

### 5-[(4-Fluorophenyl)sulfonyl]-2-propyl-N-(2-pyridin-4-ylethyl)benzenesulfonamide

The title compound was prepared from 5-[(4-fluorophenyl)sulfonyl]-2-propylbenzenesulfonyl chloride (0.38 g, 1.0 mmol) and 2-(pyridin-4-yl)ethanamine (0.25 g, 0.2.0 mmol) according to the procedure and in the same manner as described in Example 559, step c. The crude product was purified by preparative liquid chromatography on a Biotage® 40 Si column of pre-packed silica gel (90 g), eluting with a gradient of 50%-100% methyl tert-butyl ether in hexane at a flow rate of 50 mL/min, to afford, after crystallization from ethyl acetate-diethyl ether-hexane, 5-[(4-fluorophenyl)sulfonyl]-2-propyl-N-(2-pyridin-4-ylethyl)benzenesulfonamide (0.29 g, 63%), as a homogeneous, colorless, crystalline solid, m.p. 160-162° C.;

MS (+ESI), m/z: 462.8 [M+H]⁺;

HRMS: calcd for C₂₂H₂₃FN₂O₄S₂+H+, 463.11560; found (ESI, [M+H]⁺), 463.1139;

HPLC purity 100% at 210-370 nm, 8.6 min; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 876

### 5-(Phenylsulfonyl)-2-propyl-N-(2-pyridin-3-ylethyl)benzenesulfonamide

The title compound was prepared from 5-phenylsulfonyl-2-propyl-benzenesulfonyl chloride (0.36 g, 1.0 mmol) and 2-(pyridin-3-yl)ethanamine (0.25 g, 0.2.0 mmol) according to the procedure and in the same manner as described in Example 550, step c. The crude product was purified by preparative liquid chromatography on a Biotage® 40 Si column of pre-packed silica gel (90 g), eluting with a gradient of 50%-100% methyl tert-butyl ether in hexane at a flow rate of 50 mL/min, to afford 5-(phenylsulfonyl)-2-propyl-N-(2-pyridin-3-ylethyl)benzenesulfonamide (0.30 g, 68%), as a white foam;

MS (+ESI), m/z: 444.8 [M+H]⁺;

HRMS: calcd for C₂₂H₂₄N₂O₄S₂+H+, 445.12502; found (ESI, [M+H]⁺), 445.1261;

HPLC purity 98.1% at 210-370 nm, 8.6 min; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 877

### 4-[2-({[5-(phenylsulfonyl)-2-(trifluoromethyl)phenyl]sulfonyl}amino)ethyl]benzoic acid

In an analogous manner to example 654, 2-trifluoromethyl-5-(phenylsulfonyl)-benzenesulfonyl chloride and 4-(2-aminoethyl)benzoic acid were used to prepare 4-[2-({[5-(phenylsulfonyl)-2-(trifluoromethyl)phenyl]sulfonyl}amino)ethyl]benzoic acid.

MS (ESI−) m/z 512;

HPLC purity 100% at 210-370 nm, 9.1 min.; Xterra RP18, 3.5 u, 150×4.6 nm column, 1.2 mL/min, 85/15-5/95 (Ammon. Form. Buff. Ph=3.5/ACN+MeOH) for 10 min, hold 4 min.

HRMS: calcd for C₂₂H₁₈F₃NO₆S₂+H+, 514.06004; found (ESI, [M+H]⁺), 514.0597.

### EXAMPLE 878

### 4-oxo-4-[4-({[5-(phenylsulfonyl)-2-(trifluoromethyl)phenyl]sulfonyl}amino)piperidin-1-yl]butanoic acid

A stirred solution of 5-(phenylsulfonyl)-N-piperidin-4-yl-2-(trifluoromethyl)benzenesulfonamide (0.10 g, 0.22 mmol) and succinic anhydride (0.023 g, 0.23 mmol) in EtOH (1 mL) was microwave irradiated for 10 minutes at 150° C. The resulting solution was concentrated and flash column separation using 0-10% methanol/methylene chloride gradient gave 4-oxo-4-[4-({[5-(phenylsulfonyl)-2-(trifluoromethyl)phenyl]sulfonyl}amino)piperidin-1-yl]butanoic acid. (0.04 g, 33%).

MS (ESI+) m/z 549;

HPLC purity 100% at 210-370 nm, 8.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₂H₂₃F₃N₂O₇S₂+H+, 549.09715; found (ESI, [M+H]⁺), 549.095.

### EXAMPLE 879

### [4-({[5-(phenylsulfonyl)-2-(trifluoromethyl)phenyl]sulfonyl}amino)piperidin-1-yl]acetic acid

A stirred solution of 5-(phenylsulfonyl)-N-piperidin-4-yl-2-(trifluoromethyl)benzenesulfonamide (0.14 g, 0.30 mmol) and tert-butylbromoacetate (0.06 g, 0.30 mmol) and triethylamine (0.1 mL, 0.7 mmol) in THF (1 mL) was microwave irradiated for 10 minutes at 120° C. The resulting solution was concentrated and flash column separation using 0-50% ethyl acetate/hexane gradient gave a crude solid. The solid was dissolved in 4N HCl in dioxane solution (1 mL) and stirred overnight. The solution was concentrated and trituration with ethyl acetate gave [4-({[5-(phenylsulfonyl)-2-(trifluoromethyl)phenyl]sulfonyl)amino)piperidin-1-yl]acetic acid. (0.09 g, 59%).

HPLC purity 100% at 210-370 nm, 6.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₀H₂₁F₃N₂O₆S₂+H+, 507.08659; found (ESI, [M+H]⁺), 507.0854.

### EXAMPLE 880

### 2-[4-({[5-(phenylsulfonyl)-2-(trifluoromethyl)phenyl]sulfonyl}amino)piperidin-1-yl]acetamide

A stirred solution of 5-(phenylsulfonyl)-N-piperidin-4-yl-2-(trifluoromethyl)benzenesulfonamide (0.10 g, 0.21mmol) and bromoacetamide (0.03 g, 0.20 mmol) and triethylamine (0.1 mL, 0.7 mmol) in THF (1 mL) was microwave irradiated for 10 minutes at 120° C. The resulting solution was partitioned between sodium bicarbonate solution (sat.) and ethyl acetate. The organic layer was concentrated and flash column separation using 0-10% methanol/methylene chloride gradient gave 2-[4-({[5-(phenylsulfonyl)-2-(trifluoromethyl)phenyl]sulfonyl}amino)piperidin-1-yl]acetamide. (0.06 g, 57%).

HPLC purity 98.6% at 210-370 nm, 6.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₀H₂₂F₃N₃O₅S₂+H+, 506.10257; found (ESI, [M+H]⁺), 506.103.

### EXAMPLE 881

### N-(2-{methyl[(piperidin-4-ylamino)carbonyl]amino}ethyl)-5-(phenylsulfonyl)-2-(trifluoromethyl)benzenesulfonamide

To a solution of tert-butyl 4-[({methyl[2-({[5-(phenylsulfonyl)-2-(trifluoromethyl)phenyl]sulfonyl}amino)ethyl]amino}carbonyl)amino]piperidine-1-carboxylate (166.0 mg, 0.2559 mmol) from Example 866 in CH₂Cl₂ (3 mL) was added EtOAc saturated with HCl (3 mL). This mixture stood at room temperature 18 hours but solid had not formed. It was concentrated to 1 mL. EtOH (1 mL) was added. The oil/gel almost completely dissolved. The solvent was evaporated from this mixture. The residue crystallized as it stood at room temperature overnight to give 140.6 mg (94%) of N-(2-{methyl[(piperidin-4-ylamino)carbonyl]amino}ethyl)-5-(phenylsulfonyl)-2-(trifluoromethyl)benzenesulfonamide.

MS (ES+) m/z 548.8;

HRMS: calcd for C₂₂H₂₇F₃N₄O₅S₂+H+, 549.14477; found (ESI, [M+H]⁺), 549.1453.

### EXAMPLE 882

### ethyl N-({methyl[2-({[5-(phenylsulfonyl)-2-(trifluoromethyl)phenyl]sulfonyl}amino)ethyl]amino}carbonyl)glycinate

In an analogous manner to Example 826, N-[2-(methylamino)ethyl]-5-(phenylsulfonyl)-2-(trifluoromethyl)benzenesulfonamide hydrochloride from Example 799 and ethyl isocyanatoacetate were used to prepare ethyl N-({methyl[2-({[5-(phenylsulfonyl)-2-(trifluoromethyl)phenyl]sulfonyl}amino)ethyl]amino}carbonyl)glycinate.

MS (ES+) m/z 551.8;

HRMS: calcd for C₂₁H₂₄F₃N₃O₇S₂+H+, 552.10805; found (ESI, [M+H]⁺), 552.1089.

### EXAMPLE 883

### 3-({methyl[2-({[5-(phenylsulfonyl)-2-(trifluoromethyl)phenyl]sulfonyl}amino)ethyl]amino}sulfonyl)benzoic acid

To a stirred suspension of N-[2-(methylamino)ethyl]-5-(phenylsulfonyl)-2-(trifluoromethyl)benzenesulfonamide hydrochloride (111 mg, 0.242 mmol) from Example 799 in a solution of 3-(chlorosulfonyl)benzoic acid (53.3 mg, 0.242 mmol) in CH₂Cl₂ (5 mL) under N₂ at room temperature was added diisopropylethylamine (0.127 mL, 94.2 mg, 0.729 mmol). The mixture was stirred at room temperature for 3 days. It was washed twice with 2N HCl. The solution of crude was loaded directly onto a silica gel column and eluted with a gradient of hexane and ethyl acetate to give 109 mg (75%) of 3-({methyl[2-({[5-(phenylsulfonyl)-2-(trifluoromethyl)phenyl]sulfonyl}amino)ethyl]amino}sulfonyl)benzoic acid.

MS (ES+) m/z 606.7;

HRMS: calcd for C₂₃H₂₁F₃N₂O₈S₃+H+, 607.04849; found (ESI, [M+H]⁺), 607.0455.

### EXAMPLE 884

### 5-[(4-fluorophenyl)sulfonyl]-2-isopropyl-N-(2-piperidin-3-ylethyl)benzenesulfonamide

5-[(4-fluorophenyl)sulfonyl]-2-isopropyl-N-(2-pyridin-3-ylethyl)benzenesulfonamide (Example 534) (0.20 g, 0.43 mmol) was hydrogenated with palladium on carbon (70 mg) in ethanol (30 mL) at 50 psi. The reaction was filtered through Celite® and washed with ethanol. The filtrate was concentrated to give 5-[(4-fluorophenyl)sulfonyl]-2-isopropyl-N-(2-piperidin-3-ylethyl)benzenesulfonamide (0.17 g, 84%).

MS (ES+) m/z 468.8.

### EXAMPLE 885

### 5-(phenylsulfonyl)-N-[1-(2H-tetrazol-5-ylmethyl)piperidin-4-yl]-2-(trifluoromethyl)benzenesulfonamide

Step 1: A solution of 5-(phenylsulfonyl)-N-piperidin-4-yl-2-(trifluoromethyl)benzenesulfonamide (0.15 g, 0.34 mmol) and bromoacetonitrile (0.04 g, 0.34 mmol) and triethylamine (0.1 mL, 0.7 mmol) in THF (1 mL) was stirred overnight at room temperature. The mixture was concentrated and flash column separation using 0-50% ethyl acetate/hexane gradient gave crude 2-[4-({[5-(phenylsulfonyl)-2-(trifluoromethyl)phenyl]sulfonyl}amino)piperidin-1-yl]acetonitrile. (0.10 g, 62%).

Step 2: Following the same procedure described on example 863, 2-[4-({[5-(phenylsulfonyl)-2-(trifluoromethyl)phenyl]sulfonyl}amino)piperidin-1-yl]acetonitrile was used to prepare 5-(phenylsulfonyl)-N-[1-(2H-tetrazol-5-ylmethyl)piperidin-4-yl]-2-(trifluoromethyl)benzenesulfonamide.

MS (ES+) m/z 530.8;

HPLC purity 90.7% at 210-370 nm, 6.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₀H₂₁F₃N₆O₄S₂+H+, 531.10905; found (ESI, [M+H]⁺), 531.1094.

### EXAMPLE 886

### 4-oxo-4-[3-({[5-(phenylsulfonyl)-2-(trifluoromethyl)phenyl]sulfonyl}amino)pyrrolidin-1-yl]butanoic acid

In an analogous manner to example 878, 5-(phenylsulfonyl)-N-pyrrolidin-3-yl-2-(trifluoromethyl)benzenesulfonamide and succinic anhydride were used to prepare 4-oxo-4-[3-({[5-(phenylsulfonyl)-2-(trifluoromethyl)phenyl]sulfonyl}amino)pyrrolidin-1-yl]butanoic acid.

MS (ES+) m/z 534.7;

HPLC purity 96.4% at 210-370 nm, 7.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 887

### tert-butyl 4-({methyl[2-({[5-(phenylsulfonyl)-2-(trifluoromethyl)phenyl]sulfonyl}amino)ethyl]amino}carbonyl)piperazine-1-carboxylate

Step 1: In an analogous manner to Example 866, step 1, N,N′-carbonyldiimidazole and 1-tert-butyl piperazinecarboxylate were used to prepare the intermediate tert-butyl 4-(1H-imidazol-1-ylcarbonyl)piperazine-1-carboxylate.

Step 2: To a solution of tert-butyl 4-(1H-idazol-1-ylcarbonyl)piperazine-1-carboxylate (119 mg, 0.425 mmol) stirring in acetonitrile (0.79 mL) at room temperature under N₂ was added iodomethane (0.10 mL, 0.23 g, 1.6 mmol).The solution stirred at room temperature 18 hours. The acetonitrile and excess iodomethane were evaporated at reduced pressure. N-[2-(methylamino)ethyl]-5-(phenylsulfonyl)-2-(trifluoromethyl)benzenesulfonamide hydrochloride (172 mg, 0.375 mmol) from Example 799, CH₂Cl₂ (5.25 mL) and diisopropylethylamine (74 μL, 55 mg, 0.42 mmol) were added. The mixture was stirred at room temperature for 3 days. It was washed with dilute aqueous HCl (0.22 mL, 2N, 0.44 mmol diluted to <1M). An emulsion formed. The layers separated with the addition of 2N NaOH. The organic phase was loaded directly onto a silica gel column and eluted with a gradient of hexane and ethyl acetate to give 133 mg (56%) of tert-butyl 4-({methyl[2-({[5-(phenylsulfonyl)-2-(trifluoromethyl)phenyl]sulfonyl}amino)ethyl]amino}carbonyl)piperazine-1-carboxylate.

MS (ES+) m/z 634.8;

HRMS: calcd for C₂₆H₃₃F₃N₄O₇S₂+H+, 635.18155; found (ESI, [M+H]⁺

), 635.1817.

### EXAMPLE 888

### N-({methyl[2-({[5-(phenylsulfonyl)-2-(trifluoromethyl)phenyl]sulfonyl}amino)ethyl]amino}carbonyl)glycine

To a solution of ethyl N-({methyl[2-({[5-(phenylsulfonyl)-2-(trifluoromethyl)phenyl]sulfonyl}amino)ethyl]amino}carbonyl)glycinate (2.472 mg, 0.4481 mmol) from Example 882 in EtOH (6.5 mL) was added lithium hydroxide hydrate (94 mg, 2.24 mmol) in water (0.8 mL). The solution was stirred at room temperature for 2 h. The EtOH was evaporated. The residue was partitioned with EtOAc and 2N HCl. The EtOAc was washed once with water and dried over MgSO₄. Evaporation gave 236.7 mg (100%) of N-({methyl[2-({[5-(phenylsulfonyl)-2-(trifluoromethyl)phenyl]sulfonyl}amino)ethyl]amino}carbonyl)glycine.

MS (ES+) m/z 523.7;

HRMS: calcd for C₁₉H₂₀F₃N₃O₇S₂+H+, 524.07675; found (ESI, [M+H]⁺), 524.0753.

### EXAMPLE 889

### N-{[5-(phenylsulfonyl)-2-(trifluoromethyl)phenyl]sulfonyl}-□-alanine

Step 1: Following the same procedure described on example 654, 2-trifluoromethyl-5-(phenylsulfonyl)-benzenesulfonyl chloride and beta-alanine tertbutyl ester hydrochloride were used to prepare 3-(5-benzenesulfonyl-2-trifluoromethyl-benzenesulfonylamino)-propionic acid tert-butyl ester.

Step 2: Following the same procedure described on example 688, 3-(5-benzenesulfonyl-2-trifluoromethyl-benzenesulfonylamino)-propionic acid tert-butyl ester was used to prepare N-{[5-(phenylsulfonyl)-2-(trifluoromethyl)phenyl]sulfonyl}-□-alanine.

MS (ES−) m/z 435.7;

HPLC purity 100% at 210-370 nm, 8.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₆H₁₄F₃NO₆S₂+H+, 438.02874; found (ESI, [M+H]⁺), 438.0272.

### EXAMPLE 890

### 3-[4-({[5-(phenylsulfonyl)-2-(trifluoromethyl)phenyl]sulfonyl}amino)piperidin-1-yl]propanoic acid

In an analogous manner to example 879, 5-(phenylsulfonyl)-N-piperidin-4-yl-2-(trifluoromethyl)benzenesulfonamide and tert-butyl 3-bromopropionate were used to prepare 3-[4-({[5-(phenylsulfonyl)-2-(trifluoromethyl)phenyl]sulfonyl}amino)piperidin-1-yl]propanoic acid.

HPLC purity 97.6% at 210-370 nm, 6.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₁H₂₃F₃N₂O₆S₂+H+, 521.10224; found (ESI, [M+H]⁺), 521.1035.

### EXAMPLE 891

### 3-[4-({[5-(phenylsulfonyl)-2-(trifluoromethyl)phenyl]sulfonyl}amino)piperidin-1-yl]benzoic acid

To a stirred solution of 5-(phenylsulfonyl)-N-piperidin-4-yl-2-(trifluoromethyl)benzenesulfonamide (0.13 g, 0.29mmol) in methylene chloride (2 mL) under a drying tube was added 3-(tertbutoxycarbonyl)-phenylboronic acid (0.08g, 0.35 mmol), triethylamine (0.15 mL, 1.0 mmol) and copper II acetate (0.10 g, 0.58 mmol). The resulting solution was stirred room temperature for 2 days. The reaction mixture was washed with ammonium chloride solution (sat.), concentrated, and flash column separated using a 0-30% ethyl acetate/hexane gradient. The resulting material was dissolved in 4N HCl in dioxane solution (1 mL) and stirred overnight. The solution was concentrated and trituration with methylene chloride to give 3-[4-({[5-(phenylsulfonyl)-2-(trifluoromethyl)phenyl]sulfonyl}amino)piperidin-1-yl]benzoic acid. (0.026 g, 16%).

HPLC purity 100% at 210-370 nm, 9.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₅H₂₃F₃N₂O₆S₂+H+, 569.10224; found (ESI, [M+H]⁺), 569.1034.

### EXAMPLE 893

### 4-({[5-(phenylsulfonyl)-2-(trifluoromethyl)phenyl]sulfonyl}amino)butanoic acid

Step 1: Following the same procedure described on example 654, 2-trifluoromethyl-5-(phenylsulfonyl)-benzenesulfonyl chloride and 4-amino-N-butyric acid tert-butyl ester hydrochloride were used to prepare 3-(5-Benzenesulfonyl-2-trifluoromethyl-benzenesulfonylamino)-butyric acid tert-butyl ester.

Step 2: Following the same procedure described on example 688, 3-(5-Benzenesulfonyl-2-trifluoromethyl-benzenesulfonylamino)-butyric acid tert-butyl ester was used to prepare 4-({[5-(phenylsulfonyl)-2-(trifluoromethyl)phenyl]sulfonyl}amino)butanoic acid.

HPLC purity 99.1% at 210-370 nm, 8.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₇H₁₆F₃NO₆S₂+H+, 452.04439; found (ESI, [M+H]⁺), 452.0446.

### EXAMPLE 894

### 5-oxo-5-[4-({[5-(phenylsulfonyl)-2-(trifluoromethyl)phenyl]sulfonyl}amino)piperidin-1-yl]pentanoic acid

In an analogous manner to example 878, 5-(phenylsulfonyl)-N-piperidin-4-yl-2-(trifluoromethyl)benzenesulfonamide and glutaric anhydride were used to prepare 5-oxo-5-[4-({[5-(phenylsulfonyl)-2-(trifluoromethyl)phenyl]sulfonyl}amino)piperidin-1-yl]pentanoic acid.

HPLC purity 100% at 210-370 nm, 8.2 min.; Xterra RP18, 3.5 u, 150×4.6 mm column, 1.2 ml/min, 85/15-5/95 (Ammon. Form. Buff. Ph=3.5/ACN+MeOH) for 10 min, hold 4 min.

HRMS: calcd for C₂₃H₂₅F₃N₂O₇S₂+H+, 563.11280; found (ESI, [M+H]⁺), 563.1128.

### EXAMPLE 895

### 5-oxo-5-[3-({[5-(phenylsulfonyl)-2-(trifluoromethyl)phenyl]sulfonyl}amino)pyrrolidin-1-yl]pentanoic acid

In an analogous manner to example 878, 5-(phenylsulfonyl)-N-pyrrolidin-3-yl-2-(trifluoromethyl)benzenesulfonamide and glutaric anhydride were used to prepare 5-oxo-5-[3-({[5-(phenylsulfonyl)-2-(trifluoromethyl)phenyl]sulfonyl}amino)pyrrolidin-1-yl]pentanoic acid.

HPLC purity 93.7% at 210-370 nm, 8.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₂H₂₃F₃N₂O₇S₂+H+, 549.09715; found (ESI, [M+H]⁺), 549.0967.

### EXAMPLE 896

### tert-butyl 4-[({[5-(phenylsulfonyl)-2-(trifluoromethyl)phenyl]sulfonyl}amino)methyl]piperidine-1-carboxylate

In an analogous manner to example 435, 5-(phenylsulfonyl)-N-piperidin-4-yl-2-(trifluoromethyl)benzenesulfonamide and 4-aminomethyl-1-(tert-butoxycarbonyl)piperidine were used to prepare tert-butyl 4-[({[5-(phenylsulfonyl)-2-(trifluoromethyl)phenyl]sulfonyl}amino)methyl]piperidine-1-carboxylate.

MS (ES−) m/z 560.7;

HPLC purity 97.9% at 210-370 nm, 10.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₄H₂₉F₃N₂O₆S₂+H+, 563.14919; found (ESI, [M+H]⁺), 563.1487.

### EXAMPLE 897

### 4-{methyl[2-({[5-(phenylsulfonyl)-2-(trifluoromethyl)phenyl]sulfonyl}amino)ethyl]amino}-4-oxobutanoic acid

To a stirred suspension of N-[2-(methylamino)ethyl]-5-(phenylsulfonyl)-2-(trifluoromethyl)benzenesulfonamide hydrochloride (0.10 g, 0.22 mmol) from Example 799 in a solution of succinic anhydride (34 mg, 0.34 mmol) in toluene (3 mL) under N₂ at room temperature was added diisopropylethylamine (0.076 mL, 56 mg, 0.44 mmol). The mixture was stirred at 105° C. for 3 hours. An oil had formed which after cooling didn't dissolve with the addition of EtOAc saturated with HCl. The solution was decanted from the oil and saved. The oil was dissolved in methanol. The organic solutions were combined and washed with 2N HCl and water. It was loaded directly onto a Isco silica gel column and eluted with a gradient of hexane and ethyl acetate to give 45 mg (40%) of

4-{methyl[2-({[5-(phenylsulfonyl)-2-(trifluoromethyl)phenyl]sulfonyl}amino)ethyl]amino}-4-oxobutanoic acid.

MS (ES−) m/z 520.7;

HRMS: calcd for C₂₀H₂₁F₃N₂O₇S₂+H+, 523.08150; found (ESI, [M+H]⁺), 523.0803.

### EXAMPLE 898

### N-methyl-N-[2-({[5-(phenylsulfonyl)-2-(trifluoromethyl)phenyl]sulfonyl}amino)ethyl]piperazine-1-carboxamide

To a solution of tert-butyl 4-({methyl[2-({[5-(phenylsulfonyl)-2-(trifluoromethyl)phenyl]sulfonyl}amino)ethyl]amino}carbonyl)piperazine-1-carboxylate (81 mg, 0.13 mmol) from Example 887 in EtOH (1 mL) was added EtOAc saturated with HCl (1 mL). This mixture stood at room temperature 18 hours but solid had not formed. The solvent was evaporated after 4 days. The residue was dissolved in EtOH and transferred to another vial. No solid formed The solvent was evaporated and dried to give 48 mg (66%) of N-methyl-N-[2-({[5-(phenylsulfonyl)-2-(trifluoromethyl)phenyl]sulfonyl}amino)ethyl]piperazine-1-carboxamide.

MS (ES+) m/z 534.8;

HRMS: calcd for C₂₁H₂₅F₃N₄O₅S₂+H+, 535.12912; found (ESI, [M+H]⁺), 535.1285.

### EXAMPLE 899

### 5-(phenylsulfonyl)-N-(piperidin-4-ylmethyl)-2-(trifluoromethyl)benzenesulfonamide

In an analogous manner to example 688, tert-butyl 4-[({[5-(phenylsulfonyl)-2-(trifluoromethyl)phenyl]sulfonyl}amino)methyl]piperidine-1-carboxylate was used to prepare 5-(phenylsulfonyl)-N-(piperidin-4-ylmethyl)-2-(trifluoromethyl)benzenesulfonamide.

MS (ES−) m/z 460.8;

HPLC purity 98.3% at 210-370 nm, 7.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₉H₂₁F₃N₂O₄S₂+H+, 463.09676; found (ESI, [M+H]⁺), 463.0958.

### EXAMPLE 900

### N-(1-hydroxy-2,3-dihydro-1H-inden-2-yl)-5-(phenylsulfonyl)-2-(trifluoromethyl)benzenesulfonamide

In an analogous manner to example 765, 5-(phenylsulfonyl)-2-(trifluoromethyl)benzenesulfonyl chloride and 2-amino-2,3-dihydro-1H-inden-1-ol was used to prepare the title compound N-(1-hydroxy-2,3-dihydro-1H-inden-2-yl)-5-(phenylsulfonyl)-2-(trifluoromethyl)benzenesulfonamide (105.7 mg, 54.3%) as a white solid.

MS (ES−) m/z 495.7;

HPLC purity 97.7% at 210-370 nm, 9.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₂H₁₈F₃NO₅S₂+H+−H+, 497.05785; found (ESI, [M+H—H2O]+), 480.0508.

### EXAMPLE 901

### tert-butyl [trans-4-({[5-(phenylsulfonyl)-2-(trifluoromethyl)phenyl]sulfonyl}amino)cyclohexyl]carbamate

Step 1: To a stirred solution of trans-1,4-cyclohexanediamine (0.50 g, 4.4 mmol) in ethyl ether (25 mL) was added BOC anhydride (0.96 g, 4.4 mmol) and the resulting solution was stirred room temperature overnight. The mixture was filtered to give (4-aminocyclohexyl)-carbamic acid tert-butyl ester. (0.90 g, 82%).

Step 2: Following the same procedure described on example 435, 2-trifluoromethyl-5-(phenylsulfonyl)-benzenesulfonyl chloride and (4-aminocyclohexyl)-carbamic acid tert-butyl ester were used to prepare tert-butyl [trans-4-({[5-(phenylsulfonyl)-2-(trifluoromethyl)phenyl]sulfonyl}amino)cyclohexyl]carbamate.

MS (ES−) m/z 560.7;

HPLC purity 94.5% at 210-370 nm, 10.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₄H₂₉F₃N₂O₆S₂+H+, 563.14919; found (ESI, [M+H]⁺

), 563.1474.

### EXAMPLE 902

### 4-oxo-4-{4-[({[5-(phenylsulfonyl)-2-(trifluoromethyl)phenyl]sulfonyl}amino)methyl]piperidin-1-yl}butanoic acid

In an analogous manner to example 878, 5-(phenylsulfonyl)-N-(piperidin-4-ylmethyl)-2-(trifluoromethyl)benzenesulfonamide and succinic anhydride were used to prepare 4-oxo-4-{4-[({[5-(phenylsulfonyl)-2-(trifluoromethyl)phenyl]sulfonyl}amino)methyl]piperidin-1-yl}butanoic acid.

MS (ES−) m/z 560.7;

HPLC purity 90.6% at 210-370 nm, 8.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₃H₂₅F₃N₂O₇S₂+H+, 563.11280; found (ESI, [M+H]⁺), 563.1136.

### EXAMPLE 903

### 5-oxo-5-{4-[({[5-(phenylsulfonyl)-2-(trifluoromethyl)phenyl]sulfonyl}amino)methyl]piperidin-1-yl}pentanoic acid

In an analogous manner to example 878, 5-(phenylsulfonyl)-N-(piperidin-4-ylmethyl)-2-(trifluoromethyl)benzenesulfonamide and glutaric anhydride were used to prepare 5-oxo-5-{4-[({[5-(phenylsulfonyl)-2-(trifluoromethyl)phenyl]sulfonyl}amino)methyl]piperidin-1-yl}pentanoic acid.

MS (ES+) m/z 576.8;

HPLC purity 100% at 210-370 nm, 8.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₄H₂₇F₃N₂O₇S₂+H+, 577.12845; found (ESI, [M+H]⁺), 577.1257.

### EXAMPLE 904

### 3-({4-[({[5-(phenylsulfonyl)-2-(trifluoromethyl)phenyl]sulfonyl}amino)methyl]piperidin-1-yl}sulfonyl)benzoic acid

In an analogous manner to example 462, 5-(phenylsulfonyl)-N-(piperidin-4-ylmethyl)-2-(trifluoromethyl)benzenesulfonamide and 3-(chlorosulfonyl)benzoic acid were used to prepare 3-({4-[({[5-(phenylsulfonyl)-2-(trifluoromethyl)phenyl]sulfonyl}amino)methyl]piperidin-1-yl}sulfonyl)benzoic acid.

MS (ES+) m/z 646.7;

HPLC purity 92.6% at 210-370 nm, 9.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₆H₂₅F₃N₂O₈S₃+H+, 647.07979; found (ESI, [M+H]⁺), 647.0802.

### EXAMPLE 905

### N-{2-[(2-hydroxyethyl)(methyl)amino]ethyl}-5-(phenylsulfonyl)-2-(trifluoromethyl)benzenesulfonamide

To a stirred suspension of N-[2-(methylamino)ethyl]-5-(phenylsulfonyl)-2-(trifluoromethyl)benzenesulfonamide hydrochloride (65 mg, 0.14 mmol) from Example 799 in a solution of 2-bromoethanol (11 μL, 19 mg, 0.16 mmol) in CH₃CN (2 mL) under N₂ at room temperature was added diisopropylethylamine (49 μL, 36 mg, 0.28 mmol). The mixture was stirred under N₂ at 74° C. for 18 hours. The solvent was evaporated. The residue was partitioned with CH₂Cl₂ and water and washed twice with water. It was loaded directly onto a silica gel column and eluted with a gradient of hexane and ethyl acetate to give 40 mg (61%) of N-{2-[(2-hydroxyethyl)(methyl)amino]ethyl}-5-(phenylsulfonyl)-2-(trifluoromethyl)benzenesulfonamide.

MS (ES+) m/z 466.8;

HRMS: calcd for C₁₈H₂₁F₃N₂O₅S₂+H+, 467.09167; found (ESI, [M+H]⁺), 467.0922.

### EXAMPLE 906

### methyl N-methyl-N-[2-({[5-(phenylsulfonyl)-2-(trifluoromethyl)phenyl]sulfonyl}amino)ethyl]glycinate

To a stirred suspension of N-[2-(methylamino)ethyl]-5-(phenylsulfonyl)-2-(trifluoromethyl)benzenesulfonamide hydrochloride (12 mg, 0.26 mmol) from Example 799 in CH₂Cl₂ (3.5 mL) under N₂ at room temperature was added methyl bromoacetate (26 μL, 43 mg, 0.28 mmol) and diisopropylethylamine (83 μL, 62 mg, 0.48 mmol). The mixture was stirred at room temperatue for 2 days. It was washed twice with water and loaded directly onto a silica gel column and eluted with a gradient of hexane and ethyl acetate to give 0.11 g (85%) of methyl N-methyl-N-[2-({[5-(phenylsulfonyl)-2-(trifluoromethyl)phenyl]sulfonyl}amino)ethyl]glycinate.

MS (ES−) m/z 492.7;

HRMS: calcd for C₁₉H₂₁F₃N₂O₆S₂+H+, 495.08659; found (ESI, [M+H]⁺), 495.0846.

### EXAMPLE 907

### ethyl N-methyl-N-[2-({[5-(phenylsulfonyl)-2-(trifluoromethyl)phenyl]sulfonyl}amino)ethyl]-β-alaninate

To a stirred suspension of N-[2-(methylamino)ethyl]-5-(phenylsulfonyl)-2-(trifluoromethyl)benzenesulfonamide hydrochloride (0.15 g, 0.33 mmol) from Example 799 in CHCl₃ (15 mL) under N₂ at room temperature was added ethyl acrylate (48 μL, 44 mg, 0.44 mmol) and diisopropylethylamine (70 μL, 52 mg, 0.40 mmol). The mixture was stirred at 57° C. for 18 hours. Ethyl acrylate (46 μL, 42 mg, 0.42 mmol) and diisopropylethylamine (90 μL, 67 mg, 0.52 mmol) were added. The mixture was stirred at 57° C. for 2 days. It was washed with water and concentrated to 5 mL. This was loaded directly onto a Isco silica gel column and eluted with a gradient of hexane and ethyl acetate to give 0.15 g (88%) of ethyl N-methyl-N-[2-({[5-(phenylsulfonyl)-2-(trifluoromethyl)phenyl]sulfonyl}amino)ethyl]-β-alaninate.

MS (ES+) m/z 522.8;

HRMS: calcd for C₂₁H₂₅F₃N₂O₆S₂+H+, 523.11789; found (ESI, [M+H]⁺), 523.1188.

### EXAMPLE 908

### tert-butyl 2-[2-({[5-(phenylsulfonyl)-2-(trifluoromethyl)phenyl]sulfonyl}amino)ethyl]pyrrolidine-1-carboxylate

In an analogous manner to example 435, 2-trifluoromethyl-5-(phenylsulfonyl)-benzenesulfonyl chloride and 2-(aminoethyl)-1-N—BOC-pyrrolidine were used to prepare tert-butyl 2-[2-({[5-(phenylsulfonyl)-2-(trifluoromethyl)phenyl]sulfonyl}amino)ethyl]pyrrolidine-1-carboxylate.

MS (ES−) m/z 560.8;

HPLC purity 100% at 210-370 nm, 10.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 909

### tert-butyl 4-({[2-ethyl-5-(phenylsulfonyl)phenyl]sulfonyl}amino)piperidine-1-carboxylate

In an analogous manner to Example 300:

5-benzenesulfonyl-2-ethyl-benzenesulfonyl chloride and 4-amino-piperidine-1-carboxylic acid tert-butyl ester were used to prepare tert-butyl 4-({[2-ethyl-5-(phenylsulfonyl)phenyl]sulfonyl}amino)piperidine-1-carboxylate.

MS (ES−) m/z 507.1;

HPLC purity 100% at 210-370 nm, 10.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₄H₃₂N₂O₆S₂+H+, 509.17745; found (ESI, [M+H]⁺), 509.1793.

### EXAMPLE 910

### tert-butyl 4-({[2,3-dimethyl-5-(phenylsulfonyl)phenyl]sulfonyl}amino)piperidine-1-carboxylate

In an analogous manner to Example 347:

5-Benzenesulfonyl-2,3-dimethyl-benzenesulfonyl chloride and 4-amino-piperidine-1-carboxylic acid tert-butyl ester were used to prepare tert-butyl 4-({[2,3-dimethyl-5-(phenylsulfonyl)phenyl]sulfonyl}amino)piperidine-1-carboxylate.

MS (ES−) m/z 507.1;

HPLC purity 100% at 210-370 nm, 9.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₄H₃₂N₂O₆S₂+H+, 509.17745; found (ESI, [M+H]⁺), 509.1761.

### EXAMPLE 911

### tert-butyl 4-({[2,4-dimethyl-5-(phenylsulfonyl)phenyl]sulfonyl}amino)piperidine-1-carboxylate

In an analogous manner to Step 3, Example 295:

5-Benzenesulfonyl-2,4-dimethyl-benzenesulfonyl chloride and 4-amino-piperidine-1-carboxylic acid tert-butyl ester were used to prepare tert-butyl 4-({[2,4-dimethyl-5-(phenylsulfonyl)phenyl]sulfonyl}amino)piperidine-1-carboxylate.

MS (ES−) m/z 507.1;

HPLC purity 100% at 210-370 nm, 9.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₄H₃₂N₂O₆S₂+H+, 509.17745; found (ESI, [M+H]⁺), 509.1798.

### EXAMPLE 912

### tert-butyl 4-({[2-isopropyl-5-(phenylsulfonyl)phenyl]sulfonyl}amino)piperidine-1-carboxylate

In an analogous manner to Step 3, Example 295:

5-Benzenesulfonyl-2-isopropyl-benzenesulfonyl chloride and 4-amino-piperidine-1-carboxylic acid tert-butyl ester were used to prepare tert-butyl 4-({[2-isopropyl-5-(phenylsulfonyl)phenyl]sulfonyl}amino)piperidine-1-carboxylate.

MS (ES−) m/z 521.1;

HPLC purity 100% at 210-370 nm, 10.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₅H₃₄N₂O₆S₂+H+, 523.19310; found (ESI, [M+H]⁺), 523.1921.

### EXAMPLE 913

### 5-(phenylsulfonyl)-N-(2-pyrrolidin-2-ylethyl)-2-(trifluoromethyl)benzenesulfonamide

In an analogous manner to example 688, tert-butyl 2-[2-({[5-(phenylsulfonyl)-2-(trifluoromethyl)phenyl]sulfonyl}amino)ethyl]pyrrolidine-1-carboxylate was used to prepare 5-(phenylsulfonyl)-N-(2-pyrrolidin-2-ylethyl)-2-(trifluoromethyl)benzenesulfonamide.

MS (ES+) m/z 462.8;

HPLC purity 100% at 210-370 nm, 7.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₉H₂₁F₃N₂O₄S₂+H+, 463.09676; found (ESI, [M+H]⁺), 463.0961.

### EXAMPLE 914

### N-{1-[(4-hydroxyphenyl)sulfonyl]piperidin-4-yl}-5-(phenylsulfonyl)-2-(trifluoromethyl)benzenesulfonamide

Step 1: Following the same procedure described on example 462, 5-(phenylsulfonyl)-N-piperidin-4-yl-2-(trifluoromethyl)benzenesulfonamide and 4-methoxybenzene sulfonyl chloride were used to prepare N-{1-[(4-methoxyphenyl)sulfonyl]piperidin-4-yl}-5-(phenylsulfonyl)-2-(trifluoromethyl)benzenesulfonamide.

Step 2: Following the same procedure described on example 744, N-{1-[(4-methoxyphenyl)sulfonyl]piperidin-4-yl}-5-(phenylsulfonyl)-2-(trifluoromethyl)benzenesulfonamide was used to prepare N-{1-[(4-hydroxyphenyl)sulfonyl]piperidin-4-yl}-5-(phenylsulfonyl)-2-(trifluoromethyl)benzenesulfonamide.

MS (ES+) m/z 604.7;

MS (ES) m/z 604.7;

HPLC purity 100% at 210-370 nm, 9.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₄H₂₃F₃N₂O₇S₃+H+, 605.06922; found (ESI, [M+H]⁺), 605.0696.

### EXAMPLE 915

### tert-butyl 4-{[4-({[5-(phenylsulfonyl)-2-(trifluoromethyl)phenyl]sulfonylamino)piperidin-1-yl]carbonyl}piperidine-1-carboxylate

To a stirred solution of 1-BOC-piperidine-4-carboxylic acid (0.08g, 0.34 mmol) in methylene chloride (2 mL) was added triethylamine (0.1 mL, 0.7 mmol) and carbonyldiimidazole (0.06 g, 0.34 mmol). The resulting solution was stirred at room temperature for 20 minutes. To the mixture was added 5-(phenylsulfonyl)-N-piperidin-4-yl-2-(trifluoromethyl)benzenesulfonamide (0.15 g, 0.34 mmol). The resulting solution was stirred 1 hr, washed with ammonium chloride solution (sat). and concentrated. Flash column separation using 0%-50% ethyl acetate/hexane gradient gave tert-butyl 4-{[4-({[5-(phenylsulfonyl)-2-(trifluoromethyl)phenyl]sulfonyl}amino)piperidin-1-yl]carbonyl}piperidine-1-carboxylate. (0.13 g, 70%).

MS (ES−) m/z 657.8;

HPLC purity 100% at 210-370 nm, 9.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₉H₃₆F₃N₃O₇S₂+H+, 660.20195; found (ESI, [M+H]⁺), 660.2032.

### EXAMPLE 916

### N-[1-(N,N-dimethylglycyl)piperidin-4-yl]-5-(phenylsulfonyl)-2-(trifluoromethyl)benzenesulfonamide

In an analogous manner to example 462, 5-(phenylsulfonyl)-N-piperidin-4-yl-2-(trifluoromethyl)benzenesulfonamide and dimethylaminoaceyl chloride hydrochloride were used to prepare N-[1-(N,N-dimethylglycyl)piperidin-4-yl]-5-(phenylsulfonyl)-2-(trifluoromethyl)benzenesulfonamide.

MS (ES+) m/z 533.8;

HPLC purity 100% at 210-370 nm, 7.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₂H₂₆F₃N₃O₅S₂+H+, 534.13387; found (ESI, [M+H]⁺), 534.1341.

### EXAMPLE 917

### N-{1-[4-(dimethylamino)benzoyl]piperidin-4-yl}-5-(phenylsulfonyl)-2-(trifluoromethyl)benzenesulfonamide

In an analogous manner to example 462, 5-(phenylsulfonyl)-N-piperidin-4-yl-2-(trifluoromethyl)benzenesulfonamide and 4-(dimethylamino)benzoyl chloride were used to prepare N-{1-[4-(dimethylamino)benzoyl]piperidin-4-yl}-5-(phenylsulfonyl)-2-(trifluoromethyl)benzenesulfonamide.

MS (ES+) m/z 595.8;

HPLC purity 95.8% at 210-370 nm, 9.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₇H₂₈F₃N₃O₅S₂+H+, 596.14952; found (ESI, [M+H]⁺), 596.1509.

### EXAMPLE 918

### {2-[2-({[5-(phenylsulfonyl)-2-(trifluoromethyl)phenyl]sulfonyl}amino)ethyl]pyrrolidin-1-yl}acetic acid

In an analogous manner to example 879, 5-(phenylsulfonyl)-N-(2-pyrrolidin-2-ylethyl)-2-(trifluoromethyl)benzenesulfonamide and tert-butylbromoacetate were used to prepare {2-[2-({[5-(phenylsulfonyl)-2-(trifluoromethyl)phenyl]sulfonyl}amino)ethyl]pyrrolidin-1-yl}acetic acid.

MS (ES+) m/z 520.8;

HPLC purity 100% at 210-370 nm, 7.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₁H₂₃F₃N₂O₆S₂+H+, 521.10224; found (ESI, [M+H]⁺), 521.1031.

### EXAMPLE 919

### 4-oxo-4-{2-[2-({[5-(phenylsulfonyl)-2-(trifluoromethyl)phenyl]sulfonyl}amino)ethyl]pyrrolidin-1-yl}butanoic acid

In an analogous manner to example 878, 5-(phenylsulfonyl)-N-(2-pyrrolidin-2-ylethyl)-2-(trifluoromethyl)benzenesulfonamide and succinic anhydride were used to prepare 4-oxo-4-{2-[2-({[5-(phenylsulfonyl)-2-(trifluoromethyl)phenyl]sulfonyl}amino)ethyl]pyrrolidin-1-yl}butanoic acid.

MS (ES+) m/z 562.7;

HPLC purity 100% at 210-370 nm, 8.5 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₃H₂₅F₃N₂O₇S₂+H+, 563.11280; found (ESI, [M+H]⁺), 563.1114.

### EXAMPLE 920

### 3-({2-[2-({[5-(phenylsulfonyl)-2-(trifluoromethyl)phenyl]sulfonyl}amino)ethyl]pyrrolidin-1-yl)sulfonyl)benzoic acid

In an analogous manner to example 462, 5-(phenylsulfonyl)-N-(2-pyrrolidin-2-ylethyl)-2-(trifluoromethyl)benzenesulfonamide and and 3-(chlorosulfonyl)benzoic acid were used to prepare 3-({2-[2-({[5-(phenylsulfonyl)-2-(trifluoromethyl)phenyl]sulfonyl}amino)ethyl]pyrrolidin-1-yl}sulfonyl)benzoic acid.

MS (ES+) m/z 646.7;

HPLC purity 95.2% at 210-370 nm, 9.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₆H₂₅F₃N₂O₈S₃+H+, 647.07979; found (ESI, [M+H]⁺), 647.0803.

### EXAMPLE 921

### 2-ethyl-5-(phenylsulfonyl)-N-piperidin-4-ylbenzenesulfonamide

Example 909 (550 mg, 1.1 mmol) was dissolved in dry dioxane and a 4M hydrogenechloride solution in dioxane (2 mL) was added and the reaction was stirred at room temperature until the starting material was consumed as evidence by LCMS analysis. The resulting white precipitate was isolated by suction filtration and then recrystalized in hot ethanol resulting in the isolation of 2-ethyl-5-(phenylsulfonyl)-N-piperidin-4-ylbenzenesulfonamide (270 mg, 55%)

MS (ES+) m/z 408.8;

HPLC purity 100% at 210-370 nm, 6.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₉H₂₄N₂O₄S₂+H+, 409.12502; found (ESI, [M+H]⁺); 409.1242.

### EXAMPLE 922

### 2,3-dimethyl-5-(phenylsulfonyl)-N-piperidin-4-ylbenzenesulfonamide

In an analogous manner to Example 921:

Example 910 was treated with a solution of hydrogen chloride in dioxane to provide 2,3-dimethyl-5-(phenylsulfonyl)-N-piperidin-4-ylbenzenesulfonamide.

MS (ES+) m/z 408.8;

HPLC purity 100% at 210-370 nm, 6.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₉H₂₄N₂O₄S₂+H+, 409.12502; found (ESI, [M+H]⁺), 409.1246.

### EXAMPLE 923

### 2,4-dimethyl-5-(phenylsulfonyl)-N-piperidin-4-ylbenzenesulfonamide

In an analogous manner to Example 921:

Example 911 was treated with a solution of hydrogen chloride in dioxane to provide 2,4-dimethyl-5-(phenylsulfonyl)-N-piperidin-4-ylbenzenesulfonamide.

MS (ES+) m/z 408.8;

HPLC purity 100% at 210-370 nm, 6.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₉H₂₄N₂O₄S₂+H+, 409.12502; found (ESI, [M+H]⁺), 409.1241.

### EXAMPLE 924

### 2-isopropyl-5-(phenylsulfonyl)-N-piperidin-4-ylbenzenesulfonamide

In an analogous manner to Example 921:

Example 912 was treated with a solution of hydrogen chloride in dioxane to provide 2-isopropyl-5-(phenylsulfonyl)-N-piperidin-4-ylbenzenesulfonamide.

MS (ES+) m/z 422.9;

HPLC purity 100% at 210-370 nm, 7.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₀H₂₆N₂O₄S₂+H+, 423.14067; found (ESI, [M+H]⁺), 423.1404.

### EXAMPLE 925

### 5-(phenylsulfonyl)-N-piperidin-4-yl-2-propylbenzenesulfonamide

In an analogous manner as Example 909, 5-benzenesulfonyl-2-propyl-benzenesulfonyl chloride and 4-amino-piperidine-1-carboxylic acid tert-butyl ester were used to prepare tert-butyl 4-({[2-propyl-5-(phenylsulfonyl)phenyl]sulfonyl}amino)piperidine-1-carboxylate.

In an analogous manner to Example 921:

tert-butyl 4-({[2-propyl-5-(phenylsulfonyl)phenyl]sulfonyl}amino)piperidine-1-carboxylate was treated with a solution of hydrogen chloride in dioxane to provide 5-(phenylsulfonyl)-N-piperidin-4-yl-2-propylbenzenesulfonamide.

MS (ES+) m/z 422.9;

HPLC purity 100% at 210-370 nm, 7.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₀H₂₆N₂O₄S₂+H+, 423.14067; found (ESI, [M+H]⁺), 423.1412.

### EXAMPLE 926

### tert-butyl (3S)-3-[({methyl[2-({[5-(phenylsulfonyl)-2-(trifluoromethyl)phenyl]sulfonyl}amino)ethyl]amino}carbonyl)amino]pyrrolidine-1-carboxylate

Step 1: In an analogous manner to Example 866, Step 1, N,N′-carbonyldiimidazole and (S)—N—BOC-3-aminopyrrolidine were used to prepare the intermediate tert-butyl (3S)-3-[(1H-imidazol-1-ylcarbonyl)amino]pyrrolidine-1-carboxylate.

Step 2: In an analogous manner to Example 866, Step 2, N-[2-(methylamino)ethyl]-5-(phenylsulfonyl)-2-(trifluoromethyl)benzenesulfonamide hydrochloride from Example 799 and tert-butyl (3S)-3-[(1H-imidazol-1-ylcarbonyl)amino]pyrrolidine-1-carboxylate were used to prepare tert-butyl (3S)-3-[({methyl[2-({[5-(phenylsulfonyl)-2-(trifluoromethyl)phenyl]sulfonyl}amino)ethyl]amino}carbonyl)amino]pyrrolidine-1-carboxylate.

MS (ES−) m/z 632.8;

HRMS: calcd for C₂₆H₃₃F₃N₄O₇S₂+H+, 635.18155; found ESI, [M+H]⁺), 635.1805.

### EXAMPLE 927

### tert-butyl methyl[3-({[5-(phenylsulfonyl)-2-(trifluoromethyl)phenyl]sulfonyl}amino)propyl]carbamate

In an analogous manner to example 765, 5-(phenylsulfonyl)-2-(trifluoromethyl)benzenesulfonyl chloride and N-(3-Aminopropyl)-N-methylcarbamic acid tert-butyl was used to prepare the title compound tert-butyl methyl[3-({[5-(phenylsulfonyl)-2-(trifluoromethyl)phenyl]sulfonyl}amino)propyl]carbamate (1.827 g, 84%) as a white solid.

MS (ES) m/z 536.8;

HPLC purity 97.1% at 210-370 nm, 9.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₂H₂₇F₃N₂O₆S₂+H+, 537.13354; found (ESI, [M+H—C4H8]+), 481.0687.

### EXAMPLE 928

### N-[2-(4-aminophenyl)ethyl]-5-(phenylsulfonyl)-2-(trifluoromethyl)benzenesulfonamide

In an analogous manner to example 435, 2-trifluoromethyl-5-(phenylsulfonyl)-benzenesulfonyl chloride and 4-(2-aminoethyl)phenylamine were used to prepare N-[2-(4-aminophenyl)ethyl]-5-(phenylsulfonyl)-2-(trifluoromethyl)benzenesulfonamide.

MS (ES+) m/z 484.8;

HPLC purity 98.7% at 210-370 nm, 8.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₁H₁₉F₃N₂O₄S₂+H+, 485.08111; found (ESI, [M+H]⁺), 485.0799.

### EXAMPLE 929

### N-[3-(methylamino)propyl]-5-(phenylsulfonyl)-2-(trifluoromethyl)benzenesulfonamide

Tert-butyl methyl[3-({[5-(phenylsulfonyl)-2-(trifluoromethyl)phenyl]sulfonyl}amino)propyl]carbamate (1.49 g, 2.77 mmol) prepared in example 927 was taken up in 25 ml of hydrochloric saturated ethyl acetate. The hydrochloric salt of the title compound N-[3-(methylamino)propyl]-5-(phenylsulfonyl)-2-(trifluoromethyl)benzenesulfonamide (1.29 g, 98%) crashed out of solution as a white solid.

MS (ES−) m/z 434.7;

HPLC purity 98.4% at 210-370 nm, 6.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₇H₁₉F₃N₂O₄S₂+H+, 437.08111; found (ESI, [M+H]⁺), 437.0803.

### EXAMPLE 930

### 5-(phenylsulfonyl)-N-[1-(piperidin-4-ylcarbonyl)piperidin-4-yl]-2-(trifluoromethyl)benzenesulfonamide

To a stirred solution of tert-butyl 4-{[4-({[5-(phenylsulfonyl)-2-(trifluoromethyl)phenyl]sulfonyl}amino)piperidin-1-yl]carbonyl}piperidine-1-carboxylate (0.10 g, 0.15 mmol) in ethyl acetate (2 mL) was bubbled HCl gas. The resulting solution was stirred at room temperature overnight, and concentrated. Trituration in ethyl ether gave 5-(phenylsulfonyl)-N-[1-(piperidin-4-ylcarbonyl)piperidin-4-yl]-2-(trifluoromethyl)benzenesulfonamide. (0.085 g, 85%).

MS (ES+) m/z 559.8;

HPLC purity 100% at 210-370 nm, 7.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₄H₂₈F₃N₃O₅S₂+H+, 560.14952; found (ESI, [M+H]⁺), 560.1478.

### EXAMPLE 931

### tert-butyl 4-[2-({[5-(phenylsulfonyl)-2-(trifluoromethyl)phenyl]sulfonyl}amino)ethyl]piperidine-1-carboxylate

In an analogous manner to example 435, 2-trifluoromethyl-5-(phenylsulfonyl)-benzenesulfonyl chloride and 4-(aminoethyl)piperidine-1-carboxylic acid tert-butyl ester were used to prepare tert-butyl 4-[2-({[5-(phenylsulfonyl)-2-(trifluoromethyl)phenyl]sulfonyl}amino)ethyl]piperidine-1-carboxylate.

MS (ES) m/z 574.8;

HPLC purity 100% at 210-370 nm, 10.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 932

### methyl 3-({[2-isopropyl-5-(phenylsulfonyl)phenyl]sulfonyl}amino)cyclohexanecarboxylate

In an analogous manner to Step 3, Example 295:

5-Benzenesulfonyl-2-isopropyl-benzenesulfonyl chloride and methyl 3-aminocyclohexanecarboxylate were used to prepare methyl 3-({[2-isopropyl-5-(phenylsulfonyl)phenyl]sulfonyl}amino)cyclohexanecarboxylate.

MS (ES) m/z 479.9;

HPLC purity 100% at 210-370 nm, 9.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₃H₂₉NO₆S₂+H+, 480.15091; found (HRMS, [M+H]⁺), 480.1512.

### EXAMPLE 933

### methyl 4-({[2-isopropyl-5-(phenylsulfonyl)phenyl]sulfonyl}amino)cyclohexanecarboxylate

In an analogous manner to Step 3, Example 295:

5-Benzenesulfonyl-2-isopropyl-benzenesulfonyl chloride and methyl 4-aminocyclohexanecarboxylate were used to prepare methyl 4-({[2-isopropyl-5-(phenylsulfonyl)phenyl]sulfonyl}amino)cyclohexanecarboxylate.

MS (ES) m/z 479.8;

HPLC purity 94.0% at 210-370 nm, 9.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₃H₂₉NO₆S₂+H+, 480.15091; found (ESI, [M+H]⁺), 480.1503.

### EXAMPLE 934

### methyl trans-4-[({[2-isopropyl-5-(phenylsulfonyl)phenyl]sulfonyl}amino)methyl]cyclohexanecarboxylate

In an analogous manner to Step 3, Example 295:

5-Benzenesulfonyl-2-isopropyl-benzenesulfonyl chloride and methyl trans-4-(aminomethyl)-1-cyclohexanecarboxylate were used to prepare methyl trans-4-[({[2-isopropyl-5-(phenylsulfonyl)phenyl]sulfonyl}amino)methyl]cyclohexanecarboxylate.

MS (ES+) m/z 493.9;

HPLC purity 96.2% at 210-370 nm, 10.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₄H₃₁NO₆S₂+H+, 494.16656; found (ESI, [M+H]⁺), 494.1669.

### EXAMPLE 935

### 2-isopropyl-5-(phenylsulfonyl)-N-(2,2,6,6-tetramethylpiperidin-4-yl)benzenesulfonamide

In an analogous manner to Step 3, Example 295:

5-Benzenesulfonyl-2-isopropyl-benzenesulfonyl chloride and 4-amino-2,2,6,6-tetramethylpiperidine were used to prepare 2-isopropyl-5-(phenylsulfonyl)-N-(2,2,6,6-tetramethylpiperidin-4-yl)benzenesulfonamide.

MS (ES) m/z 478.9;

HPLC purity 97.9% at 210-370 nm, 7.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₄H₃₄N₂O₄S₂+H+, 479.20327; found (ESI, [M+H]⁺), 479.2044.

### EXAMPLE 936

### 2-isopropyl-5-(phenylsulfonyl)-N-(pyridin-4-ylmethyl)benzenesulfonamide

In an analogous manner to Step 3, Example 295:

5-Benzenesulfonyl-2-isopropyl-benzenesulfonyl chloride and 4-aminomethylpyridine were used to prepare 2-isopropyl-5-(phenylsulfonyl)-N-(pyridin-4-ylmethyl)benzenesulfonamide.

MS (ES) m/z 430.8;

HPLC purity 100% at 210-370 nm, 8.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₁H₂₂N₂O₄S₂+H+, 431.10937; found (ESI, [M+H]⁺), 431.1076.

### EXAMPLE 937

### N-[2-(methyl{[(3S)-pyrrolidin-3-ylamino]carbonyl}amino)ethyl]-5-(phenylsulfonyl)-2-(trifluoromethyl)benzenesulfonamide

To a solution of tert-butyl (3S)-3-[({methyl[2-({[5-(phenylsulfonyl)-2-(trifluoromethyl)phenyl]sulfonyl}amino)ethyl]amino}carbonyl)amino]pyrrolidine-1-carboxylate (0.17 g, 0.27 mmol) from Example 926 in EtOAc (1 mL) was added EtOAc saturated with HCl. This mixture stood at room temperature for 18 hours but solid had not formed. The solvent was evaporated. The residue was dissolved in EtOH. No solid formed. The solvent was evaporated. The residue was dried at 100° C. for 0.75 hours to give 0.14 g (88%) of N-[2-(methyl{[(3S)-pyrrolidin-3-ylamino]carbonyl}amino)ethyl]-5-(phenylsulfonyl)-2-(trifluoromethyl)benzenesulfonamide hydrochloride.

MS (ES+) m/z 534.8;

HRMS: calcd for C₂₁H₂₅F₃N₄O₅S₂+H+, 535.12912; found (HRMS, [M+H]⁺), 535.1287.

### EXAMPLE 938

### methyl 3-{[4-({[5-(phenylsulfonyl)-2-(trifluoromethyl)phenyl]sulfonyl}amino)piperidin-1-yl]sulfonyl}benzoate

To a stirred solution of acyl chloride (0.1 mL, 1.4 mmol) in methanol (2 mL) was added 3-{[4-({[5-(phenylsulfonyl)-2-(trifluoromethyl)phenyl]sulfonyl}amino)piperidin-1-yl]sulfonyl}benzoic acid (0.10 g, 0.16 mmol). The resulting solution was heated to 50° C. overnight and concentrated. Flash column separation using 0-10% methanol/methylene chloride gradient gave methyl 3-{[4-({[5-(phenylsulfonyl)-2-(trifluoromethyl)phenyl]sulfonyl}amino)piperidin-1-yl]sulfonyl}benzoate. (0.075 g, 74%).

MS (ES+) m/z 646.8;

HPLC purity 100% at 210-370 nm, 9.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₆H₂₅F₃N₂O₈S₃+H+, 647.07979; found (HRMS, [M+H]⁺), 647.0825.

### EXAMPLE 939

### N-[2-(4-methoxyphenyl)ethyl]-5-(phenylsulfonyl)-2-(trifluoromethyl)benzenesulfonamide

In an analogous manner to example 435, 2-trifluoromethyl-5-(phenylsulfonyl)-benzenesulfonyl chloride and 2-(4-methoxyphenyl)ethylamine were used to prepare N-[2-(4-methoxyphenyl)ethyl]-5-(phenylsulfonyl)-2-(trifluoromethyl)benzenesulfonamide.

MS (ES+) m/z 499.8;

HPLC purity 100% at 210-370 nm, 9.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₂H₂₀F₃NO₅S₂+H+, 500.08077; found (HRMS, [M+H]⁺), 500.0799.

### EXAMPLE 940

### 5-(phenylsulfonyl)-N-(2-piperidin-4-ylethyl)-2-(trifluoromethyl)benzenesulfonamide

In an analogous manner to example 930, tert-butyl 4-[2-({[5-(phenylsulfonyl)-2-(trifluoromethyl)phenyl]sulfonyl}amino)ethyl]piperidine-1-carboxylate was used to prepare 5-(phenylsulfonyl)-N-(2-piperidin-4-ylethyl)-2-(trifluoromethyl)benzenesulfonamide.

MS (ES+) m/z 476.8;

HPLC purity 97.8% at 210-370 nm, 7.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₀H₂₃F₃N₂O₄S₂+H+, 477.11241; found (HRMS, [M+H]⁺), 477.1128.

### EXAMPLE 941

### {4-[({[5-(phenylsulfonyl)-2-(trifluoromethyl)phenyl]sulfonyl}amino)methyl]piperidin-1-yl}acetic acid

In an analogous manner to example 879, 5-(phenylsulfonyl)-N-(piperidin-4-ylmethyl)-2-(trifluoromethyl)benzenesulfonamide and tert-butylbromoacetate were used to prepare {4-[({[5-(phenylsulfonyl)-2-(trifluoromethyl)phenyl]sulfonyl}amino)methyl]piperidin-1-yl}acetic acid.

MS (ES+) m/z 520.8;

HPLC purity 97.4% at 210-370 nm, 6.9 min.; Xterra RP18, 3.5 u, 150×4.6 mm column, 1.2 ml/min, 85/15-5/95 (Ammon. Form. Buff. Ph=3.5/ACN+MeOH) for 10 min, hold 4 min.

### EXAMPLE 942

### N-(4-aminobenzyl)-5-(phenylsulfonyl)-2-(trifluoromethyl)benzenesulfonamide

In an analogous manner to example 435, 2-trifluoromethyl-5-(phenylsulfonyl)-benzenesulfonyl chloride and 4-aminomethylphenylamine were used to prepare N-(4-aminobenzyl)-5-(phenylsulfonyl)-2-(trifluoromethyl)benzenesulfonamide.

MS (ES+) m/z 470.8;

HPLC purity 98.1% at 210-370 nm, 8.7 min.; Xterra RP18, 3.5 u, 150×4.6 mm column, 1.2 mL/min, 85/15-5/95 (Ammon. Bicarb Buff. Ph=9.5/ACN+MeOH) for 10 min, hold 4 min.

### EXAMPLE 943

### N-[2-(4-hydroxyphenyl)ethyl]-5-(phenylsulfonyl)-2-(trifluoromethyl)benzenesulfonamide

In an analogous manner to example 744, N-[2-(4-methoxyphenyl)ethyl]-5-(phenylsulfonyl)-2-(trifluoromethyl)benzenesulfonamide was used to prepare N-[2-(4-hydroxyphenyl)ethyl]-5-(phenylsulfonyl)-2-(trifluoromethyl)benzenesulfonamide.

MS (ES−) m/z 483.7;

HPLC purity 100% at 210-370 nm, 9.1 min.; Xterra RP18, 3.5 u, 150×4.6 mm column, 1.2 ml/min, 85/15-5/95 (Ammon. Form. Buff. Ph=3.5/ACN+MeOH) for 10 min, hold 4 min.

HRMS: calcd for C₂₁H₁₈F₃NO₅S₂+H+, 486.06512; found (HRMS, [M+H]⁺), 486.065.

### EXAMPLE 944

### {4-[2-({[5-(phenylsulfonyl)-2-(trifluoromethyl)phenyl]sulfonyl}amino)ethyl]piperidin-1-yl}acetic acid

In an analogous manner to example 879, 5-(phenylsulfonyl)-N-(2-piperidin-4-ylethyl)-2-(trifluoromethyl)benzenesulfonamide and tert-butylbromoacetate were used to prepare {4-[2-({[5-(phenylsulfonyl)-2-(trifluoromethyl)phenyl]sulfonyl}amino)ethyl]piperidin-1-yl}acetic acid.

MS (ES+) m/z 534.8;

HPLC purity 100% at 210-370 nm, 7.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₂H₂₅F₃N₂O₆S₂+H+, 535.11789; found (ESI, [M+H]⁺), 535.1189.

### EXAMPLE 945

### methyl 3-({[5-(phenylsulfonyl)-2-(trifluoromethyl)phenyl]sulfonyl}amino)cyclohexanecarboxylate

In an analogous manner to Step 3, Example 677:

5-Benzenesulfonyl-2-trifluoromethyl-benzenesulfonyl chloride and methyl 3-aminocyclohexanecarboxylate were used to prepare methyl 3-({[5-(phenylsulfonyl)-2-(trifluoromethyl)phenyl]sulfonyl}amino)cyclohexanecarboxylate.

MS (ES−) m/z 503.8;

HPLC purity 98.2% at 210-370 nm, 9.5 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₁H₂₂F₃NO₆S₂+H+, 506.09134; found (ESI, [M+H]⁺), 506.0894.

### EXAMPLE 946

### methyl 4-({[5-(phenylsulfonyl)-2-(trifluoromethyl)phenyl]sulfonyl}amino)cyclohexanecarboxylate

In an analogous manner to Step 3, Example 677:

5-Benzenesulfonyl-2-trifluoromethyl-benzenesulfonyl chloride and methyl 4-aminocyclohexanecarboxylate were used to prepare methyl 4-({[5-(phenylsulfonyl)-2-(trifluoromethyl)phenyl]sulfonyl}amino)cyclohexanecarboxylate.

MS (ES−) m/z 503.8;

HPLC purity 90.5% at 210-370 nm, 9.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₁H₂₂F₃NO₆S₂+H+, 506.09134; found (ESI, [M+H]⁺), 506.0928.

### EXAMPLE 947

### methyl trans-4-[({[5-(phenylsulfonyl)-2-(trifluoromethyl)phenyl]sulfonyl}amino)methyl]cyclohexanecarboxylate

In an analogous manner to Step 3, Example 677:

5-Benzenesulfonyl-2-trifluoromethyl-benzenesulfonyl chloride and methyl trans-4-(aminomethyl)-1-cyclohexanecarboxylate were used to prepare methyl trans-4-[({[5-(phenylsulfonyl)-2-(trifluoromethyl)phenyl]sulfonyl}amino)methyl]cyclohexanecarboxylate.

MS (ES−) m/z 517.8;

HPLC purity 98.1% at 210-370 nm, 9.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₂H₂₄F₃NO₆S₂+H+, 520.10699; found (ESI, [M+H]⁺), 520.108.

### EXAMPLE 948

### 5-(phenylsulfonyl)-N-(2,2,6,6-tetramethylpiperidin-4-yl)-2-(trifluoromethyl)benzenesulfonamide

In an analogous manner to Step 3, Example 677:

5-Benzenesulfonyl-2-trifluoromethyl-benzenesulfonyl chloride and 4-amino-2,2,6,6-tetramethylpiperidine were used to prepare 5-(phenylsulfonyl)-N-(2,2,6,6-tetramethylpiperidin-4-yl)-2-(trifluoromethyl)benzenesulfonamide.

MS (ES+) m/z 504.9;

HPLC purity 98.0% at 210-370 nm, 7.5 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₂H₂₇F₃N₂O₄S₂+H+, 505.14371; found (ESI, [M+H]⁺), 505.1442.

### EXAMPLE 949

### 5-(phenylsulfonyl)-N-(pyridin-4-ylmethyl)-2-(trifluoromethyl)benzenesulfonamide

In an analogous manner to Step 3, Example 677:

5-Benzenesulfonyl-2-trifluoromethyl-benzenesulfonyl chloride and 4-aminomethylpyridine were used to prepare 5-(phenylsulfonyl)-N-(pyridin-4-ylmethyl)-2-(trifluoromethyl)benzenesulfonamide.

MS (ES+) m/z 456.8;

HPLC purity 97.7% at 210-370 nm, 8.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₉H₁₅F₃N₂O₄S₂+H+, 457.04981; found (ESI, [M+H]⁺), 457.0482.

### EXAMPLE 950

### methyl 4-[({[5-(phenylsulfonyl)-2-(trifluoromethyl)phenyl]sulfonyl}amino)methyl]benzoate

In an analogous manner to Step 3, Example 677:

5-Benzenesulfonyl-2-trifluoromethyl-benzenesulfonyl chloride and methyl 4-aminomethylbenzoate were used to prepare methyl 4-[({[5-(phenylsulfonyl)-2-(trifluoromethyl)phenyl]sulfonyl}amino)methyl]benzoate.

MS (ES) m/z 513.7;

HPLC purity 100% at 210-370 nm, 9.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₂H₁₈F₃NO₆S₂+H+, 514.06004; found (ESI, [M+H]⁺), 514.0614.

### EXAMPLE 951

### methyl 4-[({[2-isopropyl-5-(phenylsulfonyl)phenyl]sulfonyl}amino)methyl]benzoate

In an analogous manner to Step 3, Example 295:

5-Benzenesulfonyl-2-isopropyl-benzenesulfonyl chloride and methyl 4-aminomethylbenzoate were used to prepare methyl 4-[({[2-isopropyl-5-(phenylsulfonyl)phenyl]sulfonyl}amino)methyl]benzoate.

MS (ES−) m/z 485.8;

HPLC purity 100% at 210-370 nm, 9.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₄H₂₅NO₆S₂+H+, 488.11961; found (ESI, [M+H]⁺), 488.1208.

### EXAMPLE 952

### 3-({[2-isopropyl-5-(phenylsulfonyl)phenyl]sulfonyl}amino)cyclohexanecarboxylic acid

Methyl 3-({[2-isopropyl-5-(phenylsulfonyl)phenyl]sulfonyl}amino)cyclohexanecarboxylate (example 932, 100 mg, 0.2 mmol) was dissolved in tetrahydrofuran (2 mL) and a 50% aqueous solution of sodium hydroxide (48 mg, 0.6 mmol) was added. The reaction mixture was stirred at room temperature for 18 h. The reaction was concentrated and then water (2 mL) was added followed by extraction with ethyl acetate (2×2 mL). The aqueous phase was acidified with 2N HCl and extracted with ethyl acetate which when evaporated under reduced pressure afforded 3-({[2-isopropyl-5-(phenylsulfonyl)phenyl]sulfonyl}amino)cyclohexanecarboxylic acid (92 mg, 95%) as an amorphous white solid.

MS (ES−) m/z 464.1;

HPLC purity 97.9% at 210-370 nm, 9.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₂H₂₇NO₆S₂+H+, 466.13526; found (ESI, [M+H]⁺), 466.1344.

### EXAMPLE 953

### 4-({[2-isopropyl-5-(phenylsulfonyl)phenyl]sulfonyl}amino)cyclohexanecarboxylic acid

In an analogous manner to Example 952:

methyl 4-({[2-isopropyl-(phenylsulfonyl)phenyl]sulfonyl}amino)cyclohexanecarboxylate (example 933) and 50% sodium hydroxide were used to prepare 4-({[2-isopropyl-5-(phenylsulfonyl)phenyl]sulfonyl}amino)cyclohexanecarboxylic acid.

MS (ES−) m/z 464.1;

HPLC purity 94.3% at 210-370 nm, 9.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₂H₂₇NO₆S₂+H+, 466.13526; found (ESI, [M+H]⁺), 466.1345.

### EXAMPLE 954

### trans-4-[({[2-isopropyl-5-(phenylsulfonyl)phenyl]sulfonyl}amino)methyl]cyclohexanecarboxylic acid

In an analogous manner to Example 952:

Methyl trans-4-[({[2-isopropyl-5-(phenylsulfonyl)phenyl]sulfonyl}amino)methyl]cyclohexanecarboxylate (example 934) and 50% sodium hydroxide were used to prepare trans-4-[({[2-isopropyl-5-(phenylsulfonyl)phenyl]sulfonyl}amino)methyl]cyclohexanecarboxylic acid.

MS (ES−) m/z 478.1;

HPLC purity 97.8% at 210-370 nm, 9.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 955

### tert-butyl 4-[({methyl[3-({[5-(phenylsulfonyl)-2-(trifluoromethyl)phenyl]sulfonyl}amino)propyl]amino}carbonyl)aminopiperidine-1-carboxylate

A mixture of N-[3-(methylamino)propyl]-5-(phenylsulfonyl)-2-(trifluoromethyl)benzenesulfonamide (202.5 mg, 0.43 mmol) prepared example 929 in dichloromethane (15 ml). Tert-butyl 4-(1H-imidazole-1-carboxamido)piperidine-1-carboxylate(109.4 mg, 0.37 mmol) and 1.5 eq. of N,N-Diisoprpylethylamine was syringed into the reaction vial and was allowed to stir overnight at room temperature. The product was transferred onto a 40 g Isco RediSep® Normal Phase column and was purified by automated flash chromatography using a gradient of 20% to 100% hexane/ethyl acetate. Isolation of the main component gave the title compound tert-butyl 4-[({methyl[3-({[5-(phenylsulfonyl)-2-(trifluoromethyl)phenyl]sulfonyl}amino)propyl]amino}carbonyl)amino]piperidine-1-carboxylate (217.2 mg, 77%) as a white solid.

MS (ES−) m/z 661.2;

HPLC purity 98.2% at 210-370 nm, 9.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₈H₃₇F₃N₄O₇S₂+H+, 663.21285; found (ESI, [M+H]⁺), 663.2208.

### EXAMPLE 956

### 3-{4-[({[5-(phenylsulfonyl)-2-(trifluoromethyl)phenyl]sulfonyl}amino)methyl]piperidin-1-yl}benzoic acid

In an analogous manner to example 891, 5-(phenylsulfonyl)-N-(piperidin-4-ylmethyl)-2-(trifluoromethyl)benzenesulfonamide and 3-(tertbutoxycarbonyl)-phenylboronic acid were used to prepare 3-{4-[({[5-(phenylsulfonyl)-2-(trifluoromethyl)phenyl]sulfonyl}amino)methyl]piperidin-1-yl}benzoic acid.

MS (ES+) m/z 583.1;

HPLC purity 100% at 210-370 nm, 9.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₆H₂₅F₃N₂O₆S₂+H+, 583.11789; found (ESI, [M+H]⁺), 583.1181.

### EXAMPLE 957

### 3-{4-[2-({[5-(phenylsulfonyl)-2-(trifluoromethyl)phenyl]sulfonyl}amino)ethyl]piperidin-1-yl}benzoic acid

In an analogous manner to example 891, 5-(phenylsulfonyl)-N-(2-piperidin-4-ylethyl)-2-(trifluoromethyl)benzenesulfonamide and 3-(tertbutoxycarbonyl)-phenylboronic acid were used to prepare 3-{4-[2-({[5-(phenylsulfonyl)-2-(trifluoromethyl)phenyl]sulfonyl}amino)ethyl]piperidin-1-yl}benzoic acid.

MS (ES+) m/z 597. 1;

HPLC purity 97.4% at 210-370 nm, 10.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₇H₂₇F₃N₂O₆S₂+H+, 597.13354; found (ESI, [M+H]⁺), 597.1345.

### EXAMPLE 958

### N-{1-[(6-chloropyridin-3-yl)carbonylepiperidin-4-yl}-5-(phenylsulfonyl)-2-(trifluoromethyl)benzenesulfonamide

In an analogous manner to example 462, 5-(phenylsulfonyl)-N-piperidin-4-yl-2-(trifluoromethyl)benzenesulfonamide and 6-chloronicotinyl chloride were used to prepare N-1-[(6-chloropyridin-3-yl)carbonyl]piperidin-4-yl}-5-(phenylsulfonyl)-2-(trifluoromethyl)benzenesulfonamide.

MS (ES) m/z 588.0;

HPLC purity 100% at 210-370 nm, 9.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 959

### N-(trans-4-aminocyclohexyl)-5-(phenylsulfonyl)-2-(trifluoromethyl)benzenesulfonamide

In an analogous manner to example 930, tert-butyl [trans-4-({[5-(phenylsulfonyl)-2-(trifluoromethyl)phenyl]sulfonyl}amino)cyclohexyl]carbamate was used to prepare N-(trans-4-aminocyclohexyl)-5-(phenylsulfonyl)-2-(trifluoromethyl)benzenesulfonamide.

MS (ES+) m/z 463.1;

HPLC purity 100% at 210-370 nm, 8.3 min.; Xterra RP18, 3.5 u, 150×4.6 mm column, 1.2 mL/min, 85/15-5/95 (Ammon. Bicarb Buff. Ph=9.5/ACN+MeOH) for 10 min, hold 4 min.

HRMS: calcd for C₁₉H₂₁F₃N₂O₄S₂+H+, 463.09676; found (ESI, [M+H]⁺), 463.0950.

### EXAMPLE 960

### N-[2-(4-bromophenyl)ethyl]-5-(phenylsulfonyl)-2-(trifluoromethyl)benzenesulfonamide

In an analogous manner to example 435, 2-trifluoromethyl-5-(phenylsulfonyl)-benzenesulfonyl chloride and 2-(4-bromophenyl)ethylamine were used to prepare N-[2-(4-bromophenyl)ethyl]-5-(phenylsulfonyl)-2-(trifluoromethyl)benzenesulfonamide.

MS (ES−) ) m/z 546.0;

HPLC purity 100% at 210-370 nm, 10.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 961

### tert-butyl N-methyl-N-[2-({[5-(phenylsulfonyl)-2-(trifluoromethyl)phenyl]sulfonyl}amino)ethyl]-□-alaninate

To a stirred suspension of N-[2-(methylamino)ethyl]-5-(phenylsulfonyl)-2-(trifluoromethyl)benzenesulfonamide hydrochloride (0.13 g, 0.28 mmol) from Example 799 in EtOH (8.9 mL) was added tert-butyl acrylate (0.14 mL, 0.13 g, 0.97 mmol) and diisopropylethylamine (59 μL, 44 mg, 0.34 mmol). The mixture was stirred 6 h at 76° C. under N₂. The solvent was evaporated and the residue was partitioned with water and CH₂Cl₂. The organic layer was washed with water. The solvent was evaporated. The residue was loaded directly onto a silica gel column and eluted with a gradient of hexane and ethyl acetate to give 0.10 g (64%) of tert-butyl N-methyl-N-[2-({[5-(phenylsulfonyl)-2-(trifluoromethyl)phenyl]sulfonyl}amino)ethyl)-β-alaninate.

MS (ESI+) m/z 551;

HRMS: calcd for C₂₃H₂₉F₃N₂O₆S₂+H+, 551.14919; found (ESI, [M+H]⁺), 551.1503.

### EXAMPLE 962

### tert-butyl N-methyl-N-[2-({[5-(phenylsulfonyl)-2-(trifluoromethyl)phenyl]sulfonyl}amino)ethyl]glycinate

In an analogous manner to Example 906, N-[2-(methylamino)ethyl]-5-(phenylsulfonyl)-2-(trifluoromethyl)benzenesulfonamide hydrochloride from Example 799 and tert-butyl bromoacetate were used to prepare tert-butyl N-methyl-N-[2-({[5-(phenylsulfonyl)-2-(trifluoromethyl)phenyl]sulfonyl}amino)ethyl]glycinate.

MS (ES−) m/z 534.8;

HRMS: calcd for C₂₂H₂₇F₃N₂O₆S₂+H+, 537.13354; found (ESI, [M+H]⁺), 537.1312.

### EXAMPLE 963

### 3-[({4-[({([5-(phenylsulfonyl)-2-(trifluoromethyl)phenyl]sulfonyl}amino)methyl]phenyl}amino)sulfonyl]benzoic acid

In an analogous manner to example 462, N-(4-aminobenzyl)-5-(phenylsulfonyl)-2-(trifluoromethyl)benzenesulfonamide and and 3-(chlorosulfonyl)benzoic acid were used to prepare 3-[({4-[({[5-(phenylsulfonyl)-2-(trifluoromethyl)phenyl]sulfonyl}amino)methyl]phenyl}amino)sulfonyl]benzoic acid.

HPLC purity 86.4% at 210-370 nm, 7.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₇H₂₁F₃N₂O₈S₃+H+, 655.04849; found (ESI, [M+H]⁺), 655.0472.

### EXAMPLE 964

### N-(4-bromobenzyl)-5-(phenylsulfonyl)-2-(trifluoromethyl)benzenesulfonamide

In an analogous manner to example 435, 2-trifluoromethyl-5-(phenylsulfonyl)-benzenesulfonyl chloride and 4-bromobenzyl amine were used to prepare N-(4-bromobenzyl)-5-(phenylsulfonyl)-2-(trifluoromethyl)benzenesulfonamide.

MS (ES−) m/z 531.6;

HPLC purity 97.6% at 210-370 nm, 10.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min. HPLC purity 97.6% at 210-370 nm, 10.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 965

### tert-butyl 4-{2-oxo-2-[4-({[5-(phenylsulfonyl)-2-(trifluoromethyl)phenyl]sulfonyl}amino)piperidin-1-yl]ethyl}piperidine-1-carboxylate

In an analogous manner to example 915, 5-(phenylsulfonyl)-N-piperidin-4-yl-2-(trifluoromethyl)benzenesulfonamide and 1-BOC-piperidin-4-ylacetic acid were used to prepare tert-butyl 4-{2-oxo-2-[4-({[5-(phenylsulfonyl)-2-(trifluoromethyl)phenyl]sulfonyl}amino)piperidin-1-yl]ethyl}piperidine-1-carboxylate.

HPLC purity 97.7% at 210-370 nm, 10.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₃₀H₃₈F₃N₃O₇S₂+H+, 674.21760; found (ESI, [M+H]⁺), 674.2167.

### EXAMPLE 966

### N-[2-(4-cyanophenyl)ethyl]-5-(phenylsulfonyl)-2-(trifluoromethyl)benzenesulfonamide

In an analogous manner to example 376, N-[2-(4-bromophenyl)ethyl]-5-(phenylsulfonyl)-2-(trifluoromethyl)benzenesulfonamide was used to prepare N-[2-(4-cyanophenyl)ethyl]-5-(phenylsulfonyl)-2-(trifluoromethyl)benzenesulfonamide.

HPLC purity 100% at 210-370 nm, 9.5 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₂H₁₇F₃N₂O₄S₂+H+, 495.06546; found (ESI, [M+H]⁺), 495.0669.

### EXAMPLE 967

### 3-({[5-(phenylsulfonyl)-2-(trifluoromethyl)phenyl]sulfonyl}amino)cyclohexanecarboxylic acid

In an analogous manner to Example 952:

Methyl 3-({[5-(phenylsulfonyl)-2-(trifluoromethyl)phenyl]sulfonyl}amino)cyclohexanecarboxylate (example 945) and 50% sodium hydroxide were used to prepare 3-(([5-(phenylsulfonyl)-2-(trifluoromethyl)phenyl]sulfonyl}amino)cyclohexanecarboxylic acid.

HPLC purity 98.5% at 210-370 nm, 8.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₀H₂₀F₃NO₆S₂+H+, 492.07569; found (ESI, [M+H]⁺), 492.075.

### EXAMPLE 968

### 4-({[5-(phenylsulfonyl)-2-(trifluoromethyl)phenyl]sulfonyl}amino)cyclohexanecarboxylic acid

In an analogous manner to Example 952:

Methyl 4-({[5-(phenylsulfonyl)-2-(trifluoromethyl)phenyl]sulfonyl}amino)cyclohexanecarboxylate (example 946) and 50% sodium hydroxide were used to prepare 4-({[5-(phenylsulfonyl)-2-(trifluoromethyl)phenyl]sulfonyl}amino)cyclohexanecarboxylic acid.

HPLC purity 90.5% at 210-370 nm, 8.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₀H₂₀F₃NO₆S₂+H+, 492.07569; found (ESI, [M+H]⁺), 492.0756.

### EXAMPLE 969

### trans-4-[({[5-(phenylsulfonyl)-2-(trifluoromethyl)phenyl]sulfonyl}amino)methyl]cyclohexanecarboxylic acid

In an analogous manner to Example 952:

HPLC purity 98.4% at 210-370 nm, 9.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₁H₂₂F₃NO₆S₂+H+, 506.09134; found (ESI, [M+H]⁺), 506.089.

### EXAMPLE 970

### 5-(phenylsulfonyl)-N-[1-(piperidin-4-ylacetyl)piperidin-4-yl]-2-(trifluoromethyl)benzenesulfonamide

In an analogous manner to example 930, tert-butyl 4-{2-oxo-2-[4-({[5-(phenylsulfonyl)-2-(trifluoromethyl)phenyl]sulfonyl}amino)piperidin-1-yl]ethyl}piperidine-1-carboxylate was used to prepare 5-(phenylsulfonyl)-N-[1-(piperidin-4-ylacetyl)piperidin-4-yl]-2-(trifluoromethyl)benzenesulfonamide.

MS (ES+) m/z 573.9;

HPLC purity 98.0% at 210-370 nm, 7.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₅H₃₀F₃N₃O₅S₂+H+, 574.16517; found (ESI, [M+H]⁺), 574.1636.

### EXAMPLE 971

### N-(3-{methyl[(piperidin-4-ylamino)carbonyl]amino}propyl)-5-(phenylsulfonyl)-2-(trifluoromethyl)benzenesulfonamide

Tert-butyl 4-[({methyl[3-({[5-(phenylsulfonyl)-2-(trifluoromethyl)phenyl]sulfonyl}amino)propyl]amino}carbonyl)amino]piperidine-1-carboxylate (156.3 mg, 0.23 mmol) prepared in example 955 was taken up in 25 ml of hydrochloric saturated ethyl acetate. The hydrochloric salt of the title compound N-(3-{methyl[(piperidin-4-ylamino)carbonyl]amino}propyl)-5-(phenylsulfonyl)-2-(trifluoromethyl)benzenesulfonamide (134.4 mg, 97%) crashed out of solution as a white solid.

MS (ES+) m/z 562.9;

HPLC purity 98.4% at 210-370 nm, 7.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₃H₂₉F₃N₄O₅S₂+H+, 563.16042; found (ESI, [M+H]⁺), 563.1581.

### EXAMPLE 972

### 4-[4-({[5-(phenylsulfonyl)-2-(trifluoromethyl)phenyl]sulfonyl}amino)piperidin-1-yl]benzoic acid

In an analogous manner to example 891, 5-(phenylsulfonyl)-N-piperidin-4-yl-2-(trifluoromethyl)benzenesulfonamide and 4-(tertbutoxycarbonyl)-phenylboronic acid were used to prepare 4-[4-({[5-(phenylsulfonyl)-2-(trifluoromethyl)phenyl]sulfonyl}amino)piperidin-1-yl]benzoic acid.

HRMS: calcd for C₂₅H₂₃F₃N₂O₆S₂+H+, 569.10224; found (ESI, [M+H]⁺), 569.0995.

### EXAMPLE 973

### methyl 4-[2-({[5-(phenylsulfonyl)-2-(trifluoromethyl)phenyl]sulfonyl}amino)ethyl]benzoate

Step 1: Following the same procedure described on example 938, 4-(2-aminoethyl)benzoic acid was used to prepare 4-(2-aminoethyl)benzoic acid methyl ester.

Step 2: Following the same procedure described on example 435, 2-trifluoromethyl-5-(phenylsulfonyl)-benzenesulfonyl chloride and 4-(2-aminoethyl)benzoic acid methyl ester were used to prepare methyl 4-[2-({[5-(phenylsulfonyl)-2-(trifluoromethyl)phenyl]sulfonyl}amino)ethyl]benzoate.

HPLC purity 92.4% at 210-370 nm, 8.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₃H₂₀F₃NO₆S₂+H+, 528.07569; found (ESI, [M+H]⁺), 528.0778.

### EXAMPLE 974

### tert-butyl 4-({methyl[3-({[5-(phenylsulfonyl)-2-(trifluoromethyl)phenyl]sulfonyl}amino)propyl]amino}carbonyl)piperazine-1-carboxylate

In an analogous manner to example 955, tert-butyl methyl[3-({[5-(phenylsulfonyl)-2-(trifluoromethyl)phenyl]sulfonyl}amino)propyl]carbamate (149.5 mg, 0.32mmol) prepare in example 927 and tert-butyl 4-(1H-imidazole-1-carboxamido)piperidine-1-carboxylate (78.1 mg, 0.28 mmol) was used to prepare the title compound tert-butyl 4-({methyl[3-({[5-(phenylsulfonyl)-2-(trifluoromethyl)phenyl]sulfonyl}amino)propyl]amino}carbonyl)piperazine-1-carboxylate (161.3 mg, 89%) as a white solid.

HPLC purity 96.8% at 210-370 nm, 9.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₇H₃₅F₃N₄O₇S₂+H+, 649.19720; found (ESI, [M+H]⁺), 649.1952.

### EXAMPLE 975

### N-methyl-N-[3-({[5-(phenylsulfonyl)-2-(trifluoromethyl)phenyl]sulfonyl}amino)propyl]piperazine-1-carboxamide

Tert-butyl 4-({methyl[3-({[5-(phenylsulfonyl)-2-(trifluoromethyl)phenyl]sulfonyl}amino)propyl]amino}carbonyl)piperazine-1-carboxylate (133.1 mg, 0.2 mmol) prepared in example 974 was taken up in 25 ml of hydrochloric saturated ethyl acetate. The hydrochloric salt of the title compound N-methyl-N-[3-({[5-(phenylsulfonyl)-2-(trifluoromethyl)phenyl]sulfonyl}amino)propyl]piperazine-1-carboxamide (118.7 mg, 98%) crashed out of solution as a yellow oil.

MS (ES+) m/z 549.0;

HPLC purity 98.2% at 210-370 nm, 8.0 min.; Xterra RP18, 3.5 u, 150×4.6 mm column, 1.2 mL/min, 85/15-5/95 (Ammon. Bicarb Buff. Ph=9.5/ACN+MeOH) for 10 min, hold 4 min.

HRMS: calcd for C₂₂H₂₇F₃N₄O₅S₂+H+, 549.14477; found (ESI, [M+H]⁺), 549.1434.

### EXAMPLE 976

### 4′-[({[5-(phenylsulfonyl)-2-(trifluoromethyl)phenyl]sulfonyl}amino)methyl]biphenyl-3-carboxylic acid

Step 1: To a stirred solution of N-(4-bromobenzyl)-5-(phenylsulfonyl)-2-(trifluoromethyl)benzenesulfonamide (0.15 g, 0.28 mmol) in glyme (3.5 mL) was added 3-(tert-butoxycarbonylphenyl) boronic acid (0.094 g, 0.42 mmol), Pd(PPh₃)₄ (0.03 g, 0.03 mmol), and sodium carbonate (0.09 g, 0.84 mmol) predissolved in water (0.6 mL). The resulting solution was heated to reflux for 2.5 hr, allowed to cool and extracted several times with ethyl acetate. The combined organic layers were dried over magnesium sulfate and concentrated. Flash column separation using 0%-30% ethyl acetate/hexane gradient gave 4′-[({[5-(phenylsulfonyl)-2-(trifluoromethyl)phenyl]sulfonyl}amino)methyl]biphenyl-3-carboxylic acid tert-butyl ester.

Step 2: In an analogous manner to example 930, 4′-[({[5-(phenylsulfonyl)-2-(trifluoromethyl)phenyl]sulfonyl}amino)methyl]biphenyl-3-carboxylic acid tert-butyl ester was used to prepare 4′-[({[5-(phenylsulfonyl)-2-(trifluoromethyl)phenyl]sulfonyl}amino)methyl]biphenyl-3-carboxylic acid.

MS (ES−) m/z 573.9;

HPLC purity 100% at 210-370 nm, 10.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₇H₂₀F₃NO₆S₂+H+, 576.07569; found (ESI, [M+H]⁺), 576.0751.

### EXAMPLE 977

### N-{1-[(2,5-dichloropyridin-3-yl)carbonyl]piperidin-4-yl}-5-(phenylsulfonyl)-2-(trifluoromethyl)benzenesulfonamide

In an analogous manner to example 435, 5-(phenylsulfonyl)-N-piperidin-4-yl-2-(trifluoromethyl)benzenesulfonamide and 2,5-dichloropyridine-3-carbonyl chloride were used to prepare N-{1-[(2,5-dichloropyridin-3-yl)carbonyl]piperidin-4-yl}-5-(phenylsulfonyl)-2-(trifluoromethyl)benzenesulfonamide.

MS (ES+) m/z 621.9;

HPLC purity 96.3% at 210-370 nm, 9.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₄H₂₀Cl₂F₃N₃O₅S₂+H+, 622.02463; found (ESI, [M+H]⁺), 622.0225.

### EXAMPLE 978

### N-[1-(2-chloroisonicotinoyl)piperidin-4-yl]-5-(phenylsulfonyl)-2-(trifluoromethyl)benzenesulfonamide

In an analogous manner to example 435, 5-(phenylsulfonyl)-N-piperidin-4-yl-2-(trifluoromethyl)benzenesulfonamide and 2-chloropyridine-4-carbonyl chloride were used to prepare N-[1-(2-chloroisonicotinoyl)piperidin-4-yl)-5-(phenylsulfonyl)-2-(trifluoromethyl)benzenesulfonamide.

MS (ES+) m/z 588.0;

HPLC purity 97.7% at 210-370 nm, 9.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₄H₂₁ClF₃N₃O₅S₂+H+, 588.06360; found (ESI, [M+H]⁺), 588.0627.

### EXAMPLE 979

### 4′-[({[5-(phenylsulfonyl)-2-(trifluoromethyl)phenyl]sulfonyl}amino)methyl]biphenyl-4-carboxylic acid

In an analogous manner to example 976, N-(4-bromobenzyl)-5-(phenylsulfonyl)-2-(trifluoromethyl)benzenesulfonamide and 4-(tert-butoxycarbonylphenyl) boronic acid were used to prepare 4′-[({[5-(phenylsulfonyl)-2-(trifluoromethyl)phenyl]sulfonyl}amino)methyl]biphenyl-4-carboxylic acid.

MS (ES−) m/z 573.9;

HPLC purity 100% at 210-370 nm, 10.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 980

### 4-{4-[({methyl[2-({[5-(phenylsulfonyl)-2-(trifluoromethyl)phenyl]sulfonyl}amino)ethyl]amino}carbonyl)amino]piperidin-1-yl}-4-oxobutanoic acid

In an analogous manner to Example 897, N-(2-{methyl[(piperidin-4-ylamino)carbonyl]amino}ethyl)-5-(phenylsulfonyl)-2-(trifluoromethyl)benzenesulfonamide hydrochloride from Example 881 and succinic anhydride were used to prepare crude product which was recrystallized from ethyl acetate/ethanol to give 4-{4-[({methyl[2-({[5-(phenylsulfonyl)-2-(trifluoromethyl)phenyl]sulfonyl}amino)ethyl]amino}carbonyl)amino]piperidin-1-yl}4-oxobutanoic acid.

MS (ES+) m/z 649.0;

HRMS: calcd for C₂₆H₃₁F₃N₄O₈S₂+H+, 649.16082; found (ESI, [M+H]⁺), 649.1613.

### EXAMPLE 981

### N-methyl-N-[2-({[5-(phenylsulfonyl)-2-(trifluoromethyl)phenyl]sulfonyl}amino)ethyl]glycine

To a solution of tert-butyl N-methyl-N-[2-({[5-(phenylsulfonyl)-2-(trifluoromethyl)phenyl]sulfonyl)}amino)ethyl]glycinate (87.9 mg, 0.164 mmol) from Example 962 in EtOAc (0.3 mL) was added EtOAc saturated with HCl (0.5 mL). The solution stood for 3 days and was then concentrated. NMR showed the reaction was incomplete. The mixture was dissolved in EtOAc saturated with HCl. The solution stood for 3 days and the solvent was removed by pipette. The solid was washed with EtOAc and dried to give 70.9 mg (90%) of N-methyl-N-[2-({[5-(phenylsulfonyl)-2-(trifluoromethyl)phenyl]sulfonyl}amino)ethyl]glycine hydrochloride.

MS (ES−) m/z 478.9;

HRMS: calcd for C₁₈H₁₉F₃N₂O₆S₂+H+, 481.07094; found (ESI, [M+H]⁺), 481.0704.

### EXAMPLE 982

### N-methyl-N-[2-({[5-(phenylsulfonyl)-2-(trifluoromethyl)phenyl]sulfonyl}amino)ethyl]-β-alanine

In an analogous manner to example 981, tert-butyl N-methyl-N-[2-({[5-(phenylsulfonyl)-2-(trifluoromethyl)phenyl]sulfonyl}amino)ethyl]-β-alaninate (99 mg, 0.18 mg) from Example 961 was dissolved in EtOAc (0.5 mL) saturated with HCl. After 3 days the solvent was removed by pipette. The solid was washed with EtOAc and dried to give 72 mg (81%) of N-methyl-N-[2-({[5-(phenylsulfonyl)-2-(trifluoromethyl)phenyl]sulfonyl}amino)ethyl]-β-alanine hydrochloride.

MS (ES+) m/z 495.0;

HRMS: calcd for C₁₉H₂₁F₃N₂O₆S₂+H+, 495.08659; found (ESI, [M+H]⁺), 495.088.

### EXAMPLE 983

### 4-(bromomethyl)-N-methyl-N-[2-({[5-(phenylsulfonyl)-2-(trifluoromethyl)phenyl]sulfonyl}amino)ethyl]benzamide

In an analogous manner to Example 833, N-[2-(methylamino)ethyl]-5-(phenylsulfonyl)-2-(trifluoromethyl)benzenesulfonamide hydrochloride from Example 799 and 4-bromomethyl benzoyl bromide were reacted as described in the example. After 2.5 hours the reaction mixture was washed once with water. It was loaded directly onto a silica gel column and eluted with a gradient of hexane and ethyl acetate to give 4-(bromomethyl)-N-methyl-N-[2-({[5-(phenylsulfonyl)-2-(trifluoromethyl)phenyl]sulfonyl}amino)ethyl]benzamide.

MS (ES+) m/z 618.9;

HRMS: calcd for C₂₄H₂₂BrF₃N₂O₅S₂+H+, 619.01783; found (ESI, [M+H]⁺), 619.0162.

### EXAMPLE 984

### 5-(phenylsulfonyl)-N-{2-[4-(2H-tetrazol-5-yl)phenyl]ethyl}-2-(trifluoromethyl)benzenesulfonamide

In an analogous manner to example 863, N-[2-(4-cyanophenyl)ethyl]-5-(phenylsulfonyl)-2-(trifluoromethyl)benzenesulfonamide was used to prepare 5-(phenylsulfonyl)-N-{2-[4-(2H-tetrazol-5-yl)phenyl]ethyl}-2-(trifluoromethyl)benzenesulfonamide.

MS (ES+) m/z 538.0;

HPLC purity 95.7% at 210-370 nm, 8.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₂H₁₈F₃N₅O₄S₂+H+, 538.08251; found (ESI, [M+H]⁺), 538.0812.

### EXAMPLE 985

### N-{1-[(3-cyanophenyl)sulfonyl]piperidin-4-yl}-5-(phenylsulfonyl)-2-(trifluoromethyl)benzenesulfonamide

In an analogous manner to example 462, 5-(phenylsulfonyl)-N-piperidin-4-yl-2-(trifluoromethyl)benzenesulfonamide and 3-cyanobenzenesulfonyl chloride were used to prepare N-{1-[(3-cyanophenyl)sulfonyl]piperidin-4-yl}-5-(phenylsulfonyl)-2-(trifluoromethyl)benzenesulfonamide.

MS (ES+) m/z 613.9;

HPLC purity 98.4% at 210-370 nm, 9.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 986

### N-[1-(3-cyanophenyl)piperidin-4-yl]-5-(phenylsulfonyl)-2-(trifluoromethyl)benzenesulfonamide

Step 1: A solution of 4-aminopiperidine (0.50 g, 5.0 mmol) and 3-fluorobenzonitrile (0.60 g, 5.0 mmol) in dimethylacetamide (5 mL) was heated 200° C. in a microwave for 20 minutes and partitioned between sodium bicarbonate solution (sat) and ethyl acetate. The organic layer was washed several times with water and concentrated. Flash column separation using 0%-10% methanol/methylene chloride gradient gave 3-(4-aminopiperidin-1-yl)-benzonitrile.

Step 2: In an analogous manner to example 435, 2-trifluoromethyl-5-(phenylsulfonyl)-benzenesulfonyl chloride and 3-(4-aminopiperidin-1-yl)-benzonitrile were used to prepare N-[1-(3-cyanophenyl)piperidin-4-yl]-5-(phenylsulfonyl)-2-(trifluoromethyl)benzenesulfonamide.

HRMS: calcd for C₂₅H₂₂F₃N₃O₄S₂+H+, 550.10766; found (ESI, [M+H]⁺), 550.1066;

HPLC purity 100% at 210-370 nm, 10.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 987

### N-(2-{4-[(methylsulfonyl)amino]phenyl}ethyl)-5-(phenylsulfonyl)-2-(trifluoromethyl)benzenesulfonamide

In an analogous manner to example 435, N-[2-(4-aminophenyl)ethyl]-5-(phenylsulfonyl)-2-(trifluoromethyl)benzenesulfonamide and methane sulfonyl chloride were used to prepare N-(2-{4-[(methylsulfonyl)amino]phenyl}ethyl)-5-(phenylsulfonyl)-2-(trifluoromethyl)benzenesulfonamide.

HRMS: calcd for C₂₂H₂₁F₃N₂O₆S₃+H+, 563.05866; found (ESI, [M+H]⁺), 563.0579;

HPLC purity 92.4% at 210-370 nm, 8.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 988

### tert-butyl [3-({[5-(phenylsulfonyl)-2-(trifluoromethyl)phenyl]sulfonyl}amino)propyl]carbamate

In an analogous manner to example 765, 5-(phenylsulfonyl)-2-(trifluoromethyl)benzenesulfonyl chloride (768.9 mg, 2 mmol) and tert-Butyl N-(3-aminopropyl)carbamate (525 μL, 3 mmol) was used to prepare the title compound tert-butyl [3-({[5-(phenylsulfonyl)-2-(trifluoromethyl)phenyl]sulfonyl}amino)propyl]carbamate (1.0331 g 99%) as a white foam.

MS (ES−) m/z 521.0;

HPLC purity 98.2% at 210-370 nm, 9.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₁H₂₅F₃N₂O₆S₂+H+, 523.11789; found (ESI, [M+H−tboc]+), 467.0599.

### EXAMPLE 989

### tert-butyl 4-[methyl({methyl[2-({[5-(phenylsulfonyl)-2-(trifluoromethyl)phenyl]sulfonyl}amino)ethyl]amino}carbonyl)amino]piperidine-1-carboxylate

Step 1: In an analogous manner to Example 866, Step 1, N,N′-carbonyldiimidazole and 4-N-methylamino-1-BOC-piperidine were used to prepare the intermediate tert-butyl 4-[(1H-imidazol-1-ylcarbonyl)(methyl)amino]piperidine-1-carboxylate.

Step 2: In an analogous manner to Example 887, Step 2, N-[2-(methylamino)ethyl]-5-(phenylsulfonyl)-2-(trifluoromethyl)benzenesulfonamide hydrochloride from Example 799 and tert-butyl 4-[(1H-imidazol-1-ylcarbonyl)(methyl)amino]piperidine-1-carboxylate were used to prepare tert-butyl 4-[methyl({methyl[2-({[5-(phenylsulfonyl)-2-(trifluoromethyl)phenyl]sulfonyl}amino)ethyl]amino}carbonyl)amino]piperidine-1-carboxylate.

MS (ES−) m/z 661.1;

HRMS: calcd for C₂₈H₃₇F₃N₄O₇S₂+H+, 663.21285; found (ESI, [M+H]⁺), 663.2126.

### EXAMPLE 990

### tert-butyl (3R)-3-[({methyl[2-({[5-(phenylsulfonyl)-2-(trifluoromethyl)phenyl]sulfonyl}amino)ethyl]amino}carbonyl)amino]pyrrolidine-1-carboxylate

Step 1:In an analogous manner to Example 866, Step 1, N,N′-carbonyldiimidazole and (R)-N-BOC-3-aminopyrrolidine were used to prepare the intermediate tert-butyl (3R)-3-[(1H-imidazol-1-ylcarbonyl)amino]pyrrolidine-1-carboxylate.

Step 2: In an analogous manner to Example 866, Step 2, N-[2-(methylamino)ethyl]-5-(phenylsulfonyl)-2-(trifluoromethyl)benzenesulfonamide hydrochloride from Example 799 and tert-butyl (3R)-3-[(1H-imidazol-1-ylcarbonyl)amino]pyrrolidine-1-carboxylate were used to prepare tert-butyl (3R)-3-[({methyl[2-({[5-(phenylsulfonyl)-2-(trifluoromethyl)phenyl]sulfonyl}amino)ethyl]amino}carbonyl)amino]pyrrolidine-1-carboxylate.

MS (ES−) m/z 633.0;

HRMS: calcd for C₂₆H₃₃F₃N₄O₇S₂+H+, 635.18155; found (ESI, [M+H]⁺), 635.1808.

### EXAMPLE 991

### N-(5-methoxy-2,3-dihydro-1H-inden-2-yl)-5-(phenylsulfonyl)-2-(trifluoromethyl)benzenesulfonamide

In an analogous manner to example 765, 5-(phenylsulfonyl)-2-(trifluoromethyl)benzenesulfonyl chloride (709.4 mg, 1.8 mmol) and 5-methoxy-2,3-dihydro-1H-inden-2-amine (361.1 mg, 2.2 mmol) was used to prepare the title compound N-(5-methoxy-2,3-dihydro-1H-inden-2-yl)-5-(phenylsulfonyl)-2-(trifluoromethyl)benzenesulfonamide (723.5 mg, 77%) as a yellow solid.

MS (ESI+) m/z 512;

HPLC purity 99.2% at 210-370 nm, 10.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₃H₂₀F₃NO₅S₂+H+, 512.08077; found (ESI, [M+H]⁺), 512.0795.

### EXAMPLE 992

### 4-{methyl[3-({(5-(phenylsulfonyl)-2-(trifluoromethyl)phenyl]sulfonyl}amino)propyl]amino}-4-oxobutanoic acid

A mixture of N-[3-(methylamino)propyl]-5-(phenylsulfonyl)-2-(trifluoromethyl)benzenesulfonamide (150.9 mg, 0.32 mmol) prepared example 929 in toluene (10 ml). Succinic anhydride (44.2 mg, 0.44 mmol) and 2 eq. of N,N-Diisoprpylethylamine was syringed into the reaction flask and was allowed to stir overnight at room temperature. The product was transferred onto a 12 g Isco RediSep® Normal Phase column and was purified by automated flash chromatography using a gradient of 20% to 100% hexane/ethyl acetate. Isolation of the main component gave the title compound of 4-{methyl[3-({[5-(phenylsulfonyl)-2-(trifluoromethyl)phenyl]sulfonyl}amino)propyl]amino}-4-oxobutanoic acid (121.3 mg, 71%) as an amorphous solid.

MS (ES+) m/z 536.9;

HPLC purity 93.3% at 210-370 nm, 8.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₁H₂₃F₃N₂O₇S₂+H+, 537.09715; found (ESI, [M+H]⁺), 537.0928.

### EXAMPLE 993

### N-(3-aminopropyl)-5-(phenylsulfonyl)-2-(trifluoromethyl)benzenesulfonamide

Tert-butyl [3-({[5-(phenylsulfonyl)-2-(trifluoromethyl)phenyl]sulfonyl}amino)propyl]carbamate (800 mg, 1.53 mmol) prepared in example 988 was taken up in 25 ml of hydrochloric saturated ethyl acetate. The hydrochloric salt of the title compound N-(3-aminopropyl)-5-(phenylsulfonyl)-2-(trifluoromethyl)benzenesulfonamide (697.3 mg, 99%) crashed out of solution as a white solid.

MS (ES+) m/z 423.0;

HPLC purity 100% at 210-370 nm, 6.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₆H₁₇F₃N₂O₄S₂+H+, 423.06546; found (ESI, [M+H]⁺), 423.0655.

### EXAMPLE 994: N-(3-bromobenzyl)-5-(phenylsulfonyl)-2-(trifluoromethyl)benzenesulfonamide

In an analogous manner to example 435, 2-trifluoromethyl-5-(phenylsulfonyl)-benzenesulfonyl chloride and 3-bromobenzylamine were used to prepare N-(3-bromobenzyl)-5-(phenylsulfonyl)-2-(trifluoromethyl)benzenesulfonamide.

MS (ES−) m/z 531.8;

HPLC purity 98.0% at 210-370 nm, 10.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 995: 5-(phenylsulfonyl)-N-(1-{[3-(2H-tetrazol-5-yl)phenyl]sulfonyl}piperidin-4-yl)-2-(trifluoromethyl)benzenesulfonamide

In an analogous manner to example 863, N-{1-[(3-cyanophenyl)sulfonyl]piperidin-4-yl}-5-(phenylsulfonyl)-2-(trifluoromethyl)benzenesulfonamide was used to prepare 5-(phenylsulfonyl)-N-(1-{[3-(2H-tetrazol-5-yl)phenyl]sulfonyl}piperidin-4-yl)-2-(trifluoromethyl)benzenesulfonamide.

MS (ES+) m/z 657.0;

HPLC purity 93.4% at 210-370 nm, 8.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 996:

### 3′-[({[5-(phenylsulfonyl)-2-(trifluoromethyl)phenyl]sulfonyl}amino)methyl]biphenyl-3-carboxylic acid

In an analogous manner to example 976, N-(3-bromobenzyl)-5-(phenylsulfonyl)-2-(trifluoromethyl)benzenesulfonamide and 3-(tert-butoxycarbonylphenyl) boronic acid were used to prepare 3′-[({[5-(phenylsulfonyl)-2-(trifluoromethyl)phenyl]sulfonyl}amino)methyl]biphenyl-3-carboxylic acid.

MS (ES−) m/z 573.9;

HPLC purity 100% at 210-370 nm, 10.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₇H₂₀F₃NO₆S₂+H+, 576.07569; found (ESI, [M+H]⁺), 576.0735.

### EXAMPLE 997

### 3′-[({[5-(phenylsulfonyl)-2-(trifluoromethyl)phenyl]sulfonyl}amino)methyl]biphenyl-4-carboxylic acid

In an analogous manner to example 976, N-(3-bromobenzyl)-5-(phenylsulfonyl)-2-(trifluoromethyl)benzenesulfonamide and 4-(tert-butoxycarbonylphenyl) boronic acid were used to prepare 3′-[({[5-(phenylsulfonyl)-2-(trifluoromethyl)phenyl]sulfonyl}amino)methyl]biphenyl-4-carboxylic acid.

MS (ES−) m/z 573.9;

HPLC purity 100% at 210-370 nm, 10.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₇H₂₀F₃NO₆S₂+H+, 576.07569; found (ESI, [M+H]⁺), 576.076.

### EXAMPLE 998

### N-[2-(4-{[amino(imino)methyl]amino}phenyl)ethyl]-5-(phenylsulfonyl)-2-(trifluoromethyl)benzenesulfonamide

A mixture of N-[2-(4-aminophenyl)ethyl]-5-(phenylsulfonyl)-2-(trifluoromethyl)benzenesulfonamide (0.12 g, 0.25 mmol) and 3,5-dimethylpyrazole-1-carboxamidine nitrate (0.05 g, 0.25 mmol) were heated neat at 170° C. for 10 minutes. The resulting mixture was allowed to cool and flash column separation using 0%-10% methanol/methylene chloride gradient gave N-[2-(4-{[amino(imino)methyl]amino}phenyl)ethyl]-5-(phenylsulfonyl)-2-(trifluoromethyl)benzenesulfonamide. (0.036 g, 28%).

MS (ES+) m/z 527.0;

HPLC purity 100% at 210-370 nm, 7.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₂H₂₁F₃N₄O₄S₂+H+, 527.10291; found (ESI, [M+H]⁺), 527.1037.

### EXAMPLE 999

### 5-[(4-methoxyphenyl)sulfonyl]-N-piperidin-4-yl-2-(trifluoromethyl)benzenesulfonamide

Step 1: In an analogous manner to example 820, 2-trifluoromethyl-5-(4-methoxyphenylsulfonyl)-benzenesulfonyl chloride and 4-aminopiperidine-1-carboxylic acid tert-butyl ester were used to prepare tert-butyl 4-({[5-(4-methoxyphenylsulfonyl)-2-(trifluoromethyl)phenyl]sulfonyl}amino)piperidine-1-carboxylate.

Step 2: In an analogous manner to example 930, tert-butyl 4-({[5-(4-methoxyphenylsulfonyl)-2-(trifluoromethyl)phenyl]sulfonyl}amino)piperidine-1-carboxylate was used to prepare 5-[(4-methoxyphenyl)sulfonyl]-N-piperidin-4-yl-2-(trifluoromethyl)benzenesulfonamide.

MS (ES+) m/z 479.0;

HPLC purity 95.9% at 210-370 nm, 7.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1000

### N-[1-(N-methylglycyl)piperidin-4-yl]-5-(phenylsulfonyl)-2-(trifluoromethyl)benzenesulfonamide

Step 1: In an analogous manner to example 462, 5-(phenylsulfonyl)-N-piperidin-4-yl-2-(trifluoromethyl)benzenesulfonamide and chloroacetyl chloride were used to prepare N-[1-(chloroacetyl)piperidin-4-yl]-5-(phenylsulfonyl)-2-(trifluoromethyl)benzenesulfonamide.

Step 2: To a stirred solution of N-[1-(chloroacetyl)piperidin-4-yl]-5-(phenylsulfonyl)-2-(trifluoromethyl)benzenesulfonamide (0.08 g, 0.15 mmol) in THF (1 mL) was added 33% methylamine in ethanol (0.1 mL) and the resulting solution was stirred overnight at room temperature. The mixture was concentrated and flash column separation using 0%-10% methanol/methylene chloride gradient gave N-[1-(N-methylglycyl)piperidin-4-yl]-5-(phenylsulfonyl)-2-(trifluoromethyl)benzenesulfonamide. (0.05 g, 61%).

MS (ES+) m/z 520.0;

HPLC purity 100% at 210-370 nm, 6.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₁H₂₄F₃N₃O₅S₂+H+, 520.11822; found (ESI, [M+H]⁺), 520.1174.

### EXAMPLE 1001

### 5-(phenylsulfonyl)-N-[1-(pyrrolidin-1-ylacetyl)piperidin-4-yl]-2-(trifluoromethyl)benzenesulfonamide

In an analogous manner to example 1000, N-[1-(chloroacetyl)piperidin-4-yl]-5-(phenylsulfonyl)-2-(trifluoromethyl)benzenesulfonamide and pyrrolidine were used to prepare 5-(phenylsulfonyl)-N-[1-(pyrrolidin-1-ylacetyl)piperidin-4-yl]-2-(trifluoromethyl)benzenesulfonamide.

MS (ES+) m/z 560.0;

HPLC purity 100% at 210-370 nm, 7.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1002

### N-[1-(morpholin-4-ylacetyl)piperidin-4-yl]-5-(phenylsulfonyl)-2-(trifluoromethyl)benzenesulfonamide

In an analogous manner to example 1000, N-[1-(chloroacetyl)piperidin-4-yl]-5-(phenylsulfonyl)-2-(trifluoromethyl)benzenesulfonamide and morpholine were used to prepare N-[1-(morpholin-4-ylacetyl)piperidin-4-yl]-5-(phenylsulfonyl)-2-(trifluoromethyl)benzenesulfonamide.

MS (ES+) m/z 576.0;

HPLC purity 100% at 210-370 nm, 7.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₄H₂₈F₃N₃O₆S₂+H+, 576.14444; found (ESI, [M+H]⁺), 576.1428.

### EXAMPLE 1003

### 2-[4-({[5-(phenylsulfonyl)-2-(trifluoromethyl)phenyl]sulfonyl}amino)piperidin-1-yl]propanamide

In an analogous manner to example 880, 5-(phenylsulfonyl)-N-piperidin-4-yl-2-(trifluoromethyl)benzenesulfonamide and 2-bromopropionamide were used to prepare 2-[4-({[5-(phenylsulfonyl)-2-(trifluoromethyl)phenyl]sulfonyl}amino)piperidin-1-yl]propanamide.

MS (ES+) m/z 520.0;

HPLC purity 100% at 210-370 nm, 6.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₁H₂₄F₃N₃O₅S₂+H+, 520.11822; found (ESI, [M+H]⁺), 520.1199.

### EXAMPLE 1004

### N-(5-hydroxy-2,3-dihydro-1H-inden-2-yl)-5-(phenylsulfonyl)-2-(trifluoromethyl)benzenesulfonamide

To a flame dried flask N-(5-methoxy-2,3-dihydro-1H-inden-2-yl)-5-(phenylsulfonyl)-2-(trifluoromethyl)benzenesulfonamide (262.3 mg, 0.51 mmol) was taken up in 5 ml of dichloromethane and cooled to −78° C. 1.0M Boron tribromide (3 ml, 3 mmol) in dichloromethane was syringed into the flask and then was allowed to warm to room temperature. The reaction was quenched with water, partioned between dichloromethane, dried with magnesium sulfate, and the solvent was removed under reduced pressure. The product was transferred onto a 4 g Isco RediSep® Normal Phase column and was purified by automated flash chromatography using a gradient of 20% to 100% hexane/ethyl acetate. Isolation of the main component gave the title compound N-(5-hydroxy-2,3-dihydro-1H-inden-2-yl)-5-(phenylsulfonyl)-2-(trifluoromethyl)benzenesulfonamide (232.9 mg, 91%) as a white solid.

MS (ES+) m/z 497.9;

HPLC purity 100% at 210-370 nm, 9.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₂H₁₈F₃NO₅S₂+H+, 498.06512; found (ESI, [M+H]⁺), 498.0631.

### EXAMPLE 1005

### 5-(phenylsulfonyl)-N-[1-(piperazin-1-ylacetyl)piperidin-4-yl]-2-(trifluoromethyl)benzenesulfonamide

Step 1: In an analogous manner to example 1000, N-[1-(chloroacetyl)piperidin-4-yl]-5-(phenylsulfonyl)-2-(trifluoromethyl)benzenesulfonamide and piperazine-1-carboxylic acid tert-butyl ester were used to prepare 5-(phenylsulfonyl)-N-[1-(piperazin-1-ylacetyl-4-tert-butylcarboxylate)piperidin-4-yl]-2-(trifluoromethyl)benzenesulfonamide.

Step 2: In an analogous manner to example 930, 5-(phenylsulfonyl)-N-[1-(piperazin-1-ylacetyl-4-tert-butylcarboxylate)piperidin-4-yl]-2-(trifluoromethyl)benzenesulfonamide was used to prepare 5-(phenylsulfonyl)-N-[1-(piperazin-1-ylacetyl)piperidin-4-yl]-2-(trifluoromethyl)benzenesulfonamide.

MS (ES−) m/z 572.9;

HPLC purity 100% at 210-370 nm, 7.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1006

### dimethyl [4-({methyl[2-({[5-(phenylsulfonyl)-2-(trifluoromethyl)phenyl]sulfonyl}amino)ethyl]amino}carbonyl)benzyl]phosphonate

A solution of 4-(bromomethyl)-N-methyl-N-[2-({[5-(phenylsulfonyl)-2-(trifluoromethyl)phenyl]sulfonyl}amino)ethyl]benzamide (0.311 g, 0.503 mmol) from Example 983 in trimethylphosphite (2.0 mL) was stirred at 110° C. for 6 h. The mixture was partitioned with 2N HCl and EtOAc. The organic layer was washed with water and loaded directly onto a silica gel column and eluted with a gradient of CH₂Cl₂ and CH3OH to give 86.1 mg (26%) of dimethyl [4-({methyl[2-({[5-(phenylsulfonyl)-2-(trifluoromethyl)phenyl]sulfonyl}amino)ethyl]amino}carbonyl)benzyl]phosphonate.

MS (ES+) m/z 649.0;

HRMS: calcd for C₂₆H₂₈F₃N₂O₈PS₂+H+, 649.10496; found (ESI, [M+H]⁺), 649.1035.

### EXAMPLE 1007

### N-[2-(methyl{[methyl(piperidin-4-yl)amino]carbonyl}amino)ethyl]-5-(phenylsulfonyl)-2-(trifluoromethyl)benzenesulfonamide

In an analogous manner to Example 937, tert-butyl 4-[methyl({methyl[2-({[5-(phenylsulfonyl)-2-(trifluoromethyl)phenyl]sulfonyl}amino)ethyl]amino}carbonyl)amino]piperidine-1-carboxylate from Example 989 was treated in EtOAc with HCl to give N-[2-(methyl{[methyl(piperidin-4-yl)amino]carbonyl}amino)ethyl]-5-(phenylsulfonyl)-2-(trifluoromethyl)benzenesulfonamide hydrochloride.

MS (ES+) m/z 563.2;

HRMS: calcd for C₂₃H₂₉F₃N₄O₅S₂+H+, 563.16042; found (ESI, [M+H]⁺), 563.1605.

### EXAMPLE 1008

### N-[2-(methyl{[(3R)-pyrrolidin-3-ylamino]carbonyl}amino)ethyl]-5-(phenylsulfonyl)-2-(trifluoromethyl)benzenesulfonamide

In an analogous manner to Example 937, tert-butyl (3R)-3-[({methyl[2-({[5-(phenylsulfonyl)-2-(trifluoromethyl)phenyl]sulfonyl}amino)ethyl]amino}carbonyl)amino]pyrrolidine-1-carboxylate from Example 990 was treated in EtOAc with HCl. The solvent was removed by pipette from the solid that formed. The solid was washed with EtOAc and dried to give N-[2-(methyl{[(3R)-pyrrolidin-3-ylamino]carbonyl}amino)ethyl]-5-(phenylsulfonyl)-2-(trifluoromethyl)benzenesulfonamide hydrochloride.

MS (ES+) m/z 535.1;

HRMS: calcd for C₂₁H₂₅F₃N₄O₅S₂+H+, 535.12912; found (ESI, [M+H]⁺), 535.129.

### EXAMPLE 1009

### tert-butyl (3R)-3-[({methyl[2-({[5-(phenylsulfonyl)-2-(trifluoromethyl)phenyl]sulfonyl}amino)ethyl]amino}carbonyl)amino]piperidine-1-carboxylate

Step 1: In an analogous manner to Example 866, Step1, N,N′-carbonyldiimidazole and (R)-1-BOC-3-amino-piperidine were used to prepare the intermediate tert-butyl (3R)-3-[(1H-imidazol-1-ylcarbonyl)amino]piperidine-1-carboxylate.

Step 2: In an analogous manner to Example 866, Step2, N-[2-(methylamino)ethyl]-5-(phenylsulfonyl)-2-(trifluoromethyl)benzenesulfonamide hydrochloride from Example 799 and tert-butyl (3R)-3-[(1H-imidazol-1-ylcarbonyl)amino]piperidine-1-carboxylate were used to prepare tert-butyl (3R)-3-[({methyl[2-({[5-(phenylsulfonyl)-2-(trifluoromethyl)phenyl]sulfonyl}amino)ethyl]amino}carbonyl)amino]piperidine-1-carboxylate.

MS (ES−) m/z 647.2;

HRMS: calcd for C₂₇H₃₅F₃N₄O₇S₂+H+, 649.19720; found (ESI, [M+H]⁺), 649.1984.

### EXAMPLE 1010

### tert-butyl (3S)-3-[({methyl[2-({[5-(phenylsulfonyl)-2-(trifluoromethyl)phenyl]sulfonyl}amino)ethyl]amino}carbonyl)amino]piperidine-1-carboxylate

Step 1: In an analogous manner to Example 866, Step1, N,N′-carbonyldiimidazole and (S)-1-BOC-3-amino-piperidine were used to prepare the intermediate tert-butyl (3S)-3-[(1H-imidazol-1-ylcarbonyl)amino]piperidine-1-carboxylate.

Step 2: In an analogous manner to Example 866, Step2, N-[2-(methylamino)ethyl]-5-(phenylsulfonyl)-2-(trifluoromethyl)benzenesulfonamide hydrochloride from Example 799 and tert-butyl (3S)-3-[(1H-imidazol-1-ylcarbonyl)amino]piperidine-1-carboxylate were used to prepare tert-butyl (3S)-3-[({methyl[2-({[5-(phenylsulfonyl)-2-(trifluoromethyl)phenyl]sulfonyl}amino)ethyl]amino}carbonyl)amino]piperidine-1-carboxylate.

MS (ES+) m/z 649.2;

HRMS: calcd for C₂₇H₃₅F₃N₄O₇S₂+H+, 649.19720; found (ESI, [M+H]⁺), 649.1954.

### EXAMPLE 1011

### tert-butyl (2S)-2-{[4-({[5-(phenylsulfonyl)-2-(trifluoromethyl)phenyl]sulfonyl}amino)piperidin-1-yl]carbonyl}pyrrolidine-1-carboxylate

In an analogous manner to example 915, N-(tert-butoxycarbonyl)-L-proline and 5-(phenylsulfonyl)-N-piperidin-4-yl-2-(trifluoromethyl)benzenesulfonamide were used to prepare tert-butyl (2S)-2-{[4-({[5-(phenylsulfonyl)-2-(trifluoromethyl)phenyl]sulfonyl}amino)piperidin-1-yl]carbonyl}pyrrolidine-1-carboxylate.

MS (ES−) m/z 644.2;

HPLC purity 100% at 210-370 nm, 9.5 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₈H₃₄F₃N₃O₇S₂+H+, 646.18630; found (ESI, [M+H]⁺), 646.188.

### EXAMPLE 1012

### 5-(phenylsulfonyl)-N-(1-L-prolylpiperidin-4-yl)-2-(trifluoromethyl)benzenesulfonamide

In an analogous manner to example 930, tert-butyl (2S)-2-{[4-({[5-(phenylsulfonyl)-2-(trifluoromethyl)phenyl]sulfonyl}amino)piperidin-1-yl]carbonyl}pyrrolidine-1-carboxylate was used to prepare 5-(phenylsulfonyl)-N-(1-L-prolylpiperidin-4-yl)-2-(trifluoromethyl)benzenesulfonamide.

MS (ES+) m/z 546.1;

HPLC purity 100% at 210-370 nm, 7.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₃H₂₆F₃N₃O₅S₂+H+, 546.13387; found (ESI, [M+H]⁺), 546.1358.

### EXAMPLE 1013

### N-(trans-4-hydroxycyclohexyl)-5-(phenylsulfonyl)-2-(trifluoromethyl)benzenesulfonamide

In an analogous manner to example 654, 2-trifluoromethyl-5-(phenylsulfonyl)-benzenesulfonyl chloride and trans-4-aminocyclohexanol were used to prepare N-(trans-4-hydroxycyclohexyl)-5-(phenylsulfonyl)-2-(trifluoromethyl)benzenesulfonamide.

MS (ES−) m/z 462.1;

HPLC purity 93.3% at 210-370 nm, 8.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1014

### tert-butyl (2R)-2-{[4-({[5-(phenylsulfonyl)-2-(trifluoromethyl)phenyl]sulfonyl}amino)piperidin-1-yl]carbonylpyrrolidine-1-carboxylate

In an analogous manner to example 915, N-(tert-butoxycarbonyl)-D-proline and 5-(phenylsulfonyl)-N-piperidin-4-yl-2-(trifluoromethyl)benzenesulfonamide were used to prepare tert-butyl (2R)-2-{[4-({[5-(phenylsulfonyl)-2-(trifluoromethyl)phenyl]sulfonyl}amino)piperidin-1-yl]carbonyl}pyrrolidine-1-carboxylate.

MS (ES−) m/z 644.2;

HPLC purity 100% at 210-370 nm, 9.5 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₈H₃₄F₃N₃O₇S₂+H+, 646.18630; found (ESI, [M+H]⁺), 646.1856.

### EXAMPLE 1015

### 5-(phenylsulfonyl)-N-(1-D-prolylpiperidin-4-yl)-2-(trifluoromethyl)benzenesulfonamide

In an analogous manner to example 930, tert-butyl (2R)-2-{[4-({[5-(phenylsulfonyl)-2-(trifluoromethyl)phenyl]sulfonyl}amino)piperidin-1-yl]carbonyl}pyrrolidine-1-carboxylate was used to prepare 5-(phenylsulfonyl)-N-(1-D-prolylpiperidin-4-yl)-2-(trifluoromethyl)benzenesulfonamide.

MS (ES+) m/z 546.1;

HPLC purity 97.7% at 210-370 nm, 7.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₃H₂₆F₃N₃O₅S₂+H+, 546.13387; found (ESI, [M+H]⁺), 546.1329.

### EXAMPLE 1016

### N-[4-(dimethylamino)benzyl]-5-(phenylsulfonyl)-2-(trifluoromethyl)benzenesulfonamide

In an analogous manner to example 435, 2-trifluoromethyl-5-(phenylsulfonyl)-benzenesulfonyl chloride and 4-dimethylaminobenzylamine were used to prepare N-[4-(dimethylamino)benzyl]-5-(phenylsulfonyl)-2-(trifluoromethyl)benzenesulfonamide.

MS (ES+) m/z 498.9;

HPLC purity 97.6% at 210-370 nm, 9.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₂H₂₁F₃N₂O₄S₂+H+, 499.09676; found (ESI, [M+H]⁺), 499.0995.

### EXAMPLE 1017

### 5-(phenylsulfonyl)-N-{1-[(6-pyrrolidin-1-ylpyridin-3-yl)carbonyl]piperidin-4-yl}-2-(trifluoromethyl)benzenesulfonamide

Step 1: In an analogous manner to example 435, piperidin-4-yl-carbamic acid tert-butyl ester and 2-chloropyridine-5-carbonyl chloride were used to prepare [1-(6-Chloro-pyridine-3-carbonyl)-piperidin-4-yl]-carbamic acid tert-butyl ester.

Step 2: To a stirred solution of [1-(6-Chloro-pyridine-3-carbonyl)-piperidin-4-yl]-carbamic acid tert-butyl ester (0.50 g, 1.47 mmol) in THF (1 mL) was added pyrrolidine (0.5 mL, 6.0 mmol) and the resulting solution was 160° C. in a microwave for 30 minutes and concentrated. Flash column separation using 0%-5% methanol/methylene chloride gradient gave [1-(6-Pyrrolidin-1-yl-pyridine-3-carbonyl)-piperidin-4-yl]-carbamic acid tert-butyl ester.

Step 3: In an analogous manner to example 930, [1-(6-Pyrrolidin-1-yl-pyridine-3-carbonyl)-piperidin-4-yl]-carbamic acid tert-butyl ester was used to prepare (4-Amino-piperidin-1-yl)-(6-pyrrolidin-1-yl-pyridin-3-yl)-methanone.

Step 4: In an analogous manner to example 435, 2-trifluoromethyl-5-(phenylsulfonyl)-benzenesulfonyl chloride and (4-Amino-piperidin-1-yl)-(6-pyrrolidin-1-yl-pyridin-3-yl)-methanone were used to prepare 5-(phenylsulfonyl)-N-{1-[(6-pyrrolidin-1-ylpyridin-3-yl)carbonyl]piperidin-4-yl}-2-(trifluoromethyl)benzenesulfonamide.

MS (ES+) m/z 623.0;

HPLC purity 92.8% at 210-370 nm, 8.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1018

### N-(1-{[6-(dimethylamino)pyridin-3-yl]carbonyl}piperidin-4-yl)-5-(phenylsulfonyl)-2-(trifluoromethyl)benzenesulfonamide

In an analogous manner to example 1017,

Step 2: [1-(6-Chloro-pyridine-3-carbonyl)-piperidin-4-yl]-carbamic acid tert-butyl ester and dimethylamine in THF were used to prepare [1-(6-Dimethylamino-pyridine-3-carbonyl)-piperidin-4-yl]-carbamic acid tert-butyl ester.

Step 3: In an analogous manner to example 930, [1-(6-Dimethylamino-pyridine-3-carbonyl)-piperidin-4-yl]-carbamic acid tert-butyl ester was used to prepare (4-Amino-piperidin-1-yl)-(6-dimethylamino-pyridin-3-yl)-methanone.

Step 4: In an analogous manner to example 435, 2-trifluoromethyl-5-(phenylsulfonyl)-benzenesulfonyl chloride and (4-Amino-piperidin-1-yl)-(6-dimethylamino-pyridin-3-yl)-methanone were used to prepare N-(1-{[6-(dimethylamino)pyridin-3-yl]carbonyl}piperidin-4-yl)-5-(phenylsulfonyl)-2-(trifluoromethyl)benzenesulfonamide.

MS (ES+) m/z 597.0;

HPLC purity 100% at 210-370 nm, 8.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1019

### N-[1-(2-morpholin-4-ylethyl)piperidin-4-yl]-5-(phenylsulfonyl)-2-(trifluoromethyl)benzenesulfonamide

To a stirred solution of 5-(phenylsulfonyl)-N-piperidin-4-yl-2-(trifluoromethyl)benzenesulfonamide (0.10 g, 0.223 mmol) in ethanol (1 mL) was added triethylamine (0.1 mL, 0.72 mmol) and 4-(2-chloroethyl)-morpholine hydrochloride (0.042 g, 0.223 mmol) and heated 120° C. in a microwave for 10 minutes and concentrated. Flash column separation using 0%-10% methanol/methylene chloride gradient gave N-[1-(2-morpholin-4-ylethyl)piperidin-4-yl]-5-(phenylsulfonyl)-2-(trifluoromethyl)benzenesulfonamide. (0.074 g, 59%).

MS (ES+) m/z 562.0;

HPLC purity 100% at 210-370 nm, 12.1 min.; XTerra MS C18, 5 u, 3×150 mm column, 0.5 ml/min, FA/MeOH gradient.

HRMS: calcd for C₂₄H₃₀F₃N₃O₅S₂+H+, 562.16517; found (ESI, [M+H]⁺), 562.1645.

### EXAMPLE 1020

### N-{1-[(6-oxo-1-{[5-(phenylsulfonyl)-2-(trifluoromethyl)phenyl]sulfonyl}-1,6-dihydropyridin-3-yl)carbonyl]piperidin-4-yl}-5-(phenylsulfonyl)-2-(trifluoromethyl)benzenesulfonamide

In an analogous manner to example 1017,

Step 2: [1-(6-Chloro-pyridine-3-carbonyl)-piperidin-4-yl]-carbamic acid tert-butyl ester and potassium tertbutoxide in THF were used to prepare [1-(6-tert-Butoxy-pyridine-3-carbonyl)-piperidin-4-yl]-carbamic acid tert-butyl ester.

Step 3: In an analogous manner to example 930, [1-(6-tert-Butoxy-pyridine-3-carbonyl)-piperidin-4-yl]-carbamic acid tert-butyl ester was used to prepare

5-(4-Amino-piperidine-1-carbonyl)-1H-pyridin-2-one.

Step 4: In an analogous manner to example 435, 2-trifluoromethyl-5-(phenylsulfonyl)-benzenesulfonyl chloride and 5-(4-Amino-piperidine-1-carbonyl)-1H-pyridin-2-one were used to prepare N-{1-[(6-oxo-1-{[5-(phenylsulfonyl)-2-(trifluoromethyl)phenyl]sulfonyl}-1,6-dihydropyridin-3-yl)carbonyl]piperidin-4-yl}-5-(phenylsulfonyl)-2-(trifluoromethyl)benzenesulfonamide.

MS (ES+) m/z 917.9;

HPLC purity 85.0% at 210-370 nm, 19.9 min.; XTerra MS C18, 5 u, 3×150 mm column, 0.5 mL/min, formic acid/MeOH gradient.

### EXAMPLE 1021

### N-(2,4-dimethoxybenzyl)-5-(phenylsulfonyl)-2-(trifluoromethyl)benzenesulfonamide

In an analogous manner to example 435, 2-trifluoromethyl-5-(phenylsulfonyl)-benzenesulfonyl chloride and 2,4-dimethoxybenzylamine were used to prepare N-(2,4-dimethoxybenzyl)-5-(phenylsulfonyl)-2-(trifluoromethyl)benzenesulfonamide.

MS (ES−) m/z 513.9;

HPLC purity 100.0% at 210-370 nm, 18.7 min.; Xterra MS C18, 5 u, 150×3.0 mm column, 0.5 mL/min, FA/MeOH grad.

### EXAMPLE 1022

### N-[2-(methyl{[(3R)-piperidin-3-ylamino]carbonyl}amino)ethyl]-5-(phenylsulfonyl)-2-(trifluoromethyl)benzenesulfonamide

In an analogous manner to Example 937, tert-butyl (3R)-3-[({methyl[2-({[5-(phenylsulfonyl)-2-(trifluoromethyl)phenyl]sulfonyl}amino)ethyl]amino}carbonyl)amino]piperidine-1-carboxylate from Example 1009 was used to prepare N-[2-(methyl{[(3R)-piperidin-3-ylamino]carbonyl}amino)ethyl]-5-(phenylsulfonyl)-2-(trifluoromethyl)benzenesulfonamide hydrochloride.

MS (ES+) m/z 549.0;

HRMS: calcd for C₂₂H₂₇F₃N₄O₅S₂+H+, 549.14477; found (ESI, [M+H]⁺), 549.1461.

### EXAMPLE 1023

### N-[2-(methyl{[(3S)-piperidin-3-ylamino]carbonyl}amino)ethyl]-5-(phenylsulfonyl)-2-(trifluoromethyl)benzenesulfonamide

In an analogous manner to Example 937, tert-butyl (3S)-3-[({methyl[2-({[5-(phenylsulfonyl)-2-(trifluoromethyl)phenyl]sulfonyl}amino)ethyl]amino}carbonyl)amino]piperidine-1-carboxylate from Example 1010 was used to prepare N-[2-(methyl{[(3S)-piperidin-3-ylamino]carbonyl}amino)ethyl]-5-(phenylsulfonyl)-2-(trifluoromethyl)benzenesulfonamide hydrochloride.

MS (ES+) m/z 549.1;

HRMS: calcd for C₂₂H₂₇F₃N₄O₅S₂+H+, 549.14477; found (ESI, [M+H]⁺), 549.1456.

### EXAMPLE 1024

### N-{1-[(6-phenylpyridin-3-yl)carbonyl]piperidin-4-yl}-5-(phenylsulfonyl)-2-(trifluoromethyl)benzenesulfonamide

Step 1: In an analogous manner to example 435, piperidin-4-yl-carbamic acid tert-butyl ester and 2-chloropyridine-5-carbonyl chloride were used to prepare [1-(6-Chloro-pyridine-3-carbonyl)-piperidin-4-yl]-carbamic acid tert-butyl ester.

Step 2: In an analogous manner to example 976 step 1, [1-(6-Chloro-pyridine-3-carbonyl)-piperidin-4-yl]-carbamic acid tert-butyl ester and phenyl boronic acid were used to prepare tert-butyl {1-[(6-phenylpyridin-3-yl)carbonyl]piperidin-4-yl}carbamate.

Step 3: In an analogous manner to example 930, tert-butyl {1-[(6-phenylpyridin-3-yl)carbonyl]piperidin-4-yl}carbamate was used to prepare 1-[(6-phenylpyridin-3-yl)carbonyl]piperidin-4-amine.

Step 4: In an analogous manner to example 435, 2-trifluoromethyl-5-(phenylsulfonyl)-benzenesulfonyl chloride and 1-[(6-phenylpyridin-3-yl)carbonyl]piperidin-4-amine were used to prepare N-{1-[(6-phenylpyridin-3-yl)carbonyl]piperidin-4-yl}-5-(phenylsulfonyl)-2-(trifluoromethyl)benzenesulfonamide.

MS (ES+) m/z 630.0;

HPLC purity 100.0% at 210-370 nm, 10.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₃₀H₂₆F₃N₃O₅S₂+H+, 630.13387; found (ESI, [M+H]⁺), 630.135.

### EXAMPLE 1025

### N-{1-[(6-morpholin-4-ylpyridin-3-yl)carbonyl]piperidin-4-yl}-5-(phenylsulfonyl)-2-(trifluoromethyl)benzenesulfonamide

In an analogous manner to example 1017,

Step 2: [1-(6-chloro-pyridine-3-carbonyl)-piperidin-4-yl]-carbamic acid tert-butyl ester and morpholine in THF were used to prepare tert-butyl {1-[(6-morpholin-4-ylpyridin-3-yl)carbonyl]piperidin-4-yl}carbamate.

Step 3: In an analogous manner to example 930, tert-butyl {1-[(6-morpholin-4-ylpyridin-3-yl)carbonyl]piperidin-4-yl}carbamate was used to prepare 1-[(6-morpholin-4-ylpyridin-3-yl)carbonyl]piperidin-4-amine.

Step 4: In an analogous manner to example 435, 2-trifluoromethyl-5-(phenylsulfonyl)-benzenesulfonyl chloride and 1-[(6-morpholin-4-ylpyridin-3-yl)carbonyl]piperidin-4-amine were used to prepare N-{1-[(6-morpholin-4-ylpyridin-3-yl)carbonyl]piperidin-4-yl}-5-(phenylsulfonyl)-2-(trifluoromethyl)benzenesulfonamide.

MS (ES+) m/z 639.1;

HPLC purity 98.3% at 210-370 nm, 9.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₈H₂₉F₃N₄O₆S₂+H+, 639.15534; found (ESI, [M+H]⁺), 639.1536.

### EXAMPLE 1026

### tert-butyl 4-[({[5-(phenylsulfonyl)-2-(trifluoromethyl)phenyl]sulfonyl}amino)carbonyl]piperidine-1-carboxylate

Step 1: In an analogous manner to example 930, N-(2,4-dimethoxybenzyl)-5-(phenylsulfonyl)-2-(trifluoromethyl)benzenesulfonamide and TFA were used to prepare 5-(phenylsulfonyl)-2-(trifluoromethyl)benzenesulfonamide.

Step 2: To a stirred solution of 1-BOC-piperidine-4-carboxylic acid (0.08g, 0.34 mmol) in methylene chloride (3 mL) was added DMAP (0.05 g, 0.37 mmol) and EDC (0.08 g, 0.40 mmol). The resulting solution was stirred at room temperature for 20 minutes. To the mixture was added 5-(phenylsulfonyl)-2-(trifluoromethyl)benzenesulfonamide (0.11 g, 0.30 mmol). The resulting solution was stirred 1 hr, washed with ammonium chloride solution (sat). and concentrated. Flash column separation using 0%-5% methanol/methylene chloride gradient gave tert-butyl 4-[({[5-(phenylsulfonyl)-2-(trifluoromethyl)phenyl]sulfonyl}amino)carbonyl]piperidine-1-carboxylate. (0.06 g, 34%).

MS (ES−) m/z 575.0;

HPLC purity 100.0% at 210-370 nm, 9.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1027

### N-{[5-(phenylsulfonyl)-2-(trifluoromethyl)phenyl]sulfonyl}piperidine-4-carboxamide

In an analogous manner to example 930, tert-butyl 4-[({[5-(phenylsulfonyl)-2-(trifluoromethyl)phenyl]sulfonyl}amino)carbonyl]piperidine-1-carboxylate was used to prepare N-{[5-(phenylsulfonyl)-2-(trifluoromethyl)phenyl]sulfonyl}piperidine-4-carboxamide.

MS (ES−) m/z 475.0;

HPLC purity 100.0% at 210-370 nm, 6.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₉H₁₉F₃N₂O₅S₂+H+, 477.07602; found (ESI, [M+H]⁺), 477.0748.

### EXAMPLE 1028

### methyl{[2-({[5-(phenylsulfonyl)-2-(trifluoromethyl)phenyl]sulfonyl}amino)-2,3-dihydro-1H-inden-5-yl]oxy}acetate

In an analogous manner to example 765, 5-(phenylsulfonyl)-2-(trifluoromethyl)benzenesulfonyl chloride (151.2 mg, 0.5 mmol) and methyl 2-(2-amino-2,3-dihydro-1H-inden-5-yloxy)acetate (155.3 mg, 0.6 mmol) was used to prepare the title compound methyl{[2-({[5-(phenylsulfonyl)-2-(trifluoromethyl)phenyl]sulfonyl}amino)-2,3-dihydro-1H-inden-5-yl]oxyacetate (176.2 mg, 62%) as a white solid.

MS (ES+) m/z 569.8;

HPLC purity 97.1% at 210-370 nm, 10.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₅H₂₂F₃NO₇S₂+H+, 570.08625; found (ESI, [M+H]⁺), 570.085.

### EXAMPLE 1029

### 5-(phenylsulfonyl)-N-[1-(2-pyrrolidin-1-ylisonicotinoyl)piperidin-4-yl]-2-(trifluoromethyl)benzenesulfonamide

Step 1: In an analogous manner to example 435, piperidin-4-yl-carbamic acid tert-butyl ester and 2-chloropyridine-4-carbonyl chloride were used to prepare tert-butyl [1-(2-chloroisonicotinoyl)piperidin-4-yl]carbamate.

Step 2: In an analogous manner to example 1017 step 2, tert-butyl [1-(2-chloroisonicotinoyl)piperidin-4-yl]carbamate and pyrrolidine in THF were used to prepare tert-butyl [1-(2-pyrrolidin-1-ylisonicotinoyl)piperidin-4-yl]carbamate.

Step 3: In an analogous manner to example 930. tert-butyl [1-(2-pyrrolidin-1-ylisonicotinoyl)piperidin-4-yl]carbamate was used to prepare

1-(2-pyrrolidin-1-ylisonicotinoyl)piperidin-4-amine.

Step 4: In an analogous manner to example 435, 2-trifluoromethyl-5-(phenylsulfonyl)-benzenesulfonyl chloride and 1-(2-pyrrolidin-1-ylisonicotinoyl)piperidin-4-amine were used to prepare 5-(phenylsulfonyl)-N-[1-(2-pyrrolidin-1-ylisonicotinoyl)piperidin-4-yl]-2-(trifluoromethyl)benzenesulfonamide.

MS (ES+) m/z 623.1;

HPLC purity 98.5% at 210-370 nm, 8.5 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₈H₂₉F₃N₄O₅S₂+H+, 623.16042; found (ESI, [M+H]⁺), 623.1596.

### EXAMPLE 1030

### N-[(6-chloropyridin-3-yl)methyl]-5-(phenylsulfonyl)-2-(trifluoromethyl)benzenesulfonamide

In an analogous manner to example 435, 2-trifluoromethyl-5-(phenylsulfonyl)-benzenesulfonyl chloride and 5-(aminomethyl)-2-chloropyridine were used to prepare N-[(6-chloropyridin-3-yl)methyl]-5-(phenylsulfonyl)-2-(trifluoromethyl)benzenesulfonamide.

MS (ES+) m/z 490.8;

HPLC purity 98.4% at 210-370 nm, 9.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₉H₁₄ClF₃N₂O₄S₂+H+, 491.01083; found (ESI, [M+H]⁺), 491.0101.

### EXAMPLE 1031

### {[2-({[5-(phenylsulfonyl)-2-(trifluoromethyl)phenyl]sulfonyl}amino)-2,3-dihydro-1H-inden-5-yl]oxy}acetic acid

Methyl {[2-({[5-(phenylsulfonyl)-2-(trifluoromethyl)phenyl]sulfonyl}amino)-2,3-dihydro-1H-inden-5-yl]oxy}acetate (125.3 mg, 0.2 mmol) prepare in example 1028 was taken up in 12.5 ml of a solution of 2:2:1 tetrahydrofuran:methanol:water. To the stirring solution 2N sodium hydroxide (220 μL, 0.44 mmol) was syringed into the reaction flask and allowed to stir overnight at room temperature. 2N Hydrochloric acid (230 μL, 0.46 mmol) was syringed into the reaction flask that allowed the title compound {[2-({[5-(phenylsulfonyl)-2-(trifluoromethyl)phenyl]sulfonyl}amino)-2,3-dihydro-1H-inden-5-yl]oxy}acetic acid (117 mg, 96%) to crash out of solution as a white solid.

MS (ES+) m/z 556.0;

HPLC purity 100.0% at 210-370 nm, 9.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₄H₂₀F₃NO₇S₂+H+, 556.07060; found (ESI, [M+H]⁺), 556.0712.

### EXAMPLE 1032

### 5-(phenylsulfonyl)-N-[(6-pyrrolidin-1-ylpyridin-3-yl)methyl]-2-(trifluoromethyl)benzenesulfonamide

In an analogous manner to example 1017 step 2, N-[(6-chloropyridin-3-yl)methyl]-5-(phenylsulfonyl)-2-(trifluoromethyl)benzenesulfonamide and pyrrolidine were used to prepare 5-(phenylsulfonyl)-N-[(6-pyrrolidin-1-ylpyridin-3-yl)methyl]-2-(trifluoromethyl)benzenesulfonamide.

MS (ES+) m/z 526.0;

HPLC purity 95.8% at 210-370 nm, 7.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1033

### N-[(6-morpholin-4-ylpyridin-3-yl)methyl]-5-(phenylsulfonyl)-2-(trifluoromethyl)benzenesulfonamide

In an analogous manner to example 1017 step 2, N-[(6-chloropyridin-3-yl)methyl]-5-(phenylsulfonyl)-2-(trifluoromethyl)benzenesulfonamide and morpholine were used to prepare N-[(6-morpholin-4-ylpyridin-3-yl)methyl]-5-(phenylsulfonyl)-2-(trifluoromethyl)benzenesulfonamide.

MS (ES+) m/z 542.0;

HPLC purity 100.0% at 210-370 nm, 8.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₃H₂₂F₃N₃O₅S₂+H+, 542.10257; found (ESI, [M+H]⁺), 542.1033.

### EXAMPLE 1034

### 5-[(3-bromophenyl)sulfonyl]-N-piperidin-4-yl-2-(trifluoromethyl)benzenesulfonamide

To a stirred solution of 5-(phenylsulfonyl)-N-piperidin-4-yl-2-(trifluoromethyl)benzenesulfonamide (0.80 g, 1.78 mmol) in conc. sulfuric acid (13 mL) and water (2 mL) was added NBS (0.32 g, 1.80 mmol) portionwise and the resulting solution was stirred overnight at room temperature. The solution was neutralized with aquous sodium hydroxide to pH ˜8 and extracted several times with ethyl acetate. The combined organic layers were dried over magnesium sulfate and concentrated. Achiral preparative super-critical fluid chromatography using 15% methanol/85% CO₂ with 0.2% dimethylethylamine gave 5-[(3-bromophenyl)sulfonyl]-N-piperidin-4-yl-2-(trifluoromethyl)benzenesulfonamide. (0.52 g, 55%).

MS (ES+) m/z 526.8;

HPLC purity 100.0% at 210-370 nm, 8.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₈H₁₈BrF₃N₂O₄S₂+H+, 526.99162; found (ESI, [M+H]⁺), 526.9941.

### EXAMPLE 1035

### N-[(1R*, 5S*)-8-Methyl-8-azabicyclo[3.2.1]oct-3-yl]-5-(phenylsulphonyl)-2-(trifluoromethyl)benzenesulfonamide

A stirred solution of 5-(phenylsulfonyl)-2-(trifluoromethyl)benzenesulfonyl chloride (0.38 g, 1.0 mmol) in 3:1 dichloromethane-acetonitrile (10 mL) was treated under nitrogen with endo-8-methyl-8-azabicyclo[3.2.1]octan-3-amine dihydrochloride (0.42 g, 2.0 mmol) and N-ethyl-N-isopropylpropan-2-amine (0.52 g, 4.0 mmol). The reaction was stirred for 18 hours at room temperature. The crude product was purified by reverse phase preparative liquid chromatography on an Xterra MSC18, 50×250 mm prep column, eluting with a mixture of 60:40 water-methanol containing 0.1% formic acid at a flow rate of 100 mL/min, to afford, after concentration of the solvent and extraction with ethyl acetate (3×), an oil. The oil was crystallized from diethyl ether to afford N-[(1R*, 5S*)-8-methyl-8-azabicyclo[3.2.1]oct-3-yl]-5-(phenylsulfonyl)-2-(trifluoromethyl)benzenesulfonamide (0.09 g, 20%), as a homogeneous, colorless, crystalline solid, m.p. 154-57° C.;

MS (−ESI), m/z: 486.9 [M−H]⁻;

HRMS: calcd for C₂₁H₂₃F₃N₂O₄S₂+H+, 489.11241; found (ESI, [M+H]⁺), 489.1109.

HPLC purity 99.5% at 210-370 nm, 7.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1036

### [4-({methyl[2-({[5-(phenylsulfonyl)-2-(trifluoromethyl)phenyl]sulfonyl}amino)ethyl]amino}carbonyl)benzyl]phosphonic acid

To a stirring solution of dimethyl [4-({methyl[2-({[5-(phenylsulfonyl)-2-(trifluoromethyl)phenyl]sulfonyl}amino)ethyl]amino}carbonyl)benzyl]phosphonate (0.282 g, 0.435 mmol) from Example 1006 in CH₂Cl₂ (5 mL) under N₂ at room temperature was added iodotrimethylsilane (0.13 mL, 0.18 g, 0.91 mmol). The mixture was stirred 2 days. The solvent and volatile components were evaporated. The residue was dissolved in CH₃OH (3 mL) and stirred overnight. Evaporation of the CH₃OH gave 0.06 g (22%) of [4-({methyl[2-({[5-(phenylsulfonyl)-2-(trifluoromethyl)phenyl]sulfonyl}amino)ethyl]amino}carbonyl)benzyl]phosphonic acid.

MS (ES+) m/z 620.8;

HRMS: calcd for C₂₄H₂₄F₃N₂O₈PS₂+H+, 621.07365; found (ESI, [M+H]⁺), 621.0723.

### EXAMPLE 1037

### methyl 2-({[5-(phenylsulfonyl)-2-(trifluoromethyl)phenyl]sulfonyl}amino)indane-5-carboxylate

In an analogous manner to example 765, 5-(phenylsulfonyl)-2-(trifluoromethyl)benzenesulfonyl chloride (98.3 mg, 0.33 mmol) and methyl 2-amino-2,3-dihydro-1H-indene-5-carboxylate (74.7 mg, 0.39 mmol) was used to prepare the title compound methyl 2-({[5-(phenylsulfonyl)-2-(trifluoromethyl)phenyl]sulfonyl}amino)indane-5-carboxylate (79.1 mg, 45%) as a white solid.

MS (ES−) m/z 537.7;

HPLC purity 100.0% at 210-370 nm, 10.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₄H₂₀F₃NO₆S₂+H+, 540.07569; found (ESI, [M+H]⁺), 540.0762.

### EXAMPLE 1038

### (2R)-2-[4-({[5-(phenylsulfonyl)-2-(trifluoromethyl)phenyl]sulfonyl}amino)piperidin-1-yl]propanamide

In an analogous manner to example 880, 5-(phenylsulfonyl)-N-piperidin-4-yl-2-(trifluoromethyl)benzenesulfonamide and 2-bromopropionamide were used to prepare 2-[4-({[5-(phenylsulfonyl)-2-(trifluoromethyl)phenyl]sulfonyl}amino)piperidin-1-yl]propanamide. This was chiral separated using column AD-H at 30% isopropyl alcohol to give (2R)-2-[4-({[5-(phenylsulfonyl)-2-(trifluoromethyl)phenyl]sulfonyl}amino)piperidin-1-yl]propanamide.

MS (ES+) m/z 520.0;

HPLC purity 100.0% at 210-370 nm, 7.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₁H₂₄F₃N₃O₅S₂+H+, 520.11822; found (ESI, [M+H]⁺), 520.1199.

### EXAMPLE 1039

### (2S)-2-[4-({[5-(phenylsulfonyl)-2-(trifluoromethyl)phenyl]sulfonyl}amino)piperidin-1-yl]propanamide

In an analogous manner to example 880, 5-(phenylsulfonyl)-N-piperidin-4-yl-2-(trifluoromethyl)benzenesulfonamide and 2-bromopropionamide were used to prepare 2-[4-({[5-(phenylsulfonyl)-2-(trifluoromethyl)phenyl]sulfonyl}amino)piperidin-1-yl]propanamide. This was chiral separated using column AD-H at 30% isopropyl alcohol to give (2S)-2-[4-({[5-(phenylsulfonyl)-2-(trifluoromethyl)phenyl]sulfonyl}amino)piperidin-1-yl]propanamide.

MS (ES+) m/z 520.1;

HPLC purity 100.0% at 210-370 nm, 7.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₁H₂₄F₃N₃O₅S₂+H+, 520.11822; found (ESI, [M+H]⁺), 520.118.

### EXAMPLE 1040

### N-{1-[(6-oxo-1,6-dihydropyridin-3-yl)carbonyl]piperidin-4-yl}-5-(phenylsulfonyl)-2-(trifluoromethyl)benzenesulfonamide

In an analogous manner to example 1017,

Step 2: [1-(6-Chloro-pyridine-3-carbonyl)-piperidin-4-yl]-carbamic acid tert-butyl ester and potassium tertbutoxide in THF were used to prepare [1-(6-tert-Butoxy-pyridine-3-carbonyl)-piperidin-4-yl]-carbamic acid tert-butyl ester.

Step 3: In an analogous manner to example 435, 2-trifluoromethyl-5-(phenylsulfonyl)-benzenesulfonyl chloride and 5-(4-Amino-piperidine-1-carbonyl)-1H-pyridin-2-one were used to prepare N-{1-[(6-tert-butoxypyridin-3-yl)carbonyl]piperidin-4-yl}-5-(phenylsulfonyl)-2-(trifluoromethyl)benzenesulfonamide.

Step 4: In an analogous manner to example 930, N-{1-[(6-tert-butoxypyridin-3-yl)carbonyl]piperidin-4-yl}-5-(phenylsulfonyl)-2-(trifluoromethyl)benzenesulfonamide was used to prepare N-{1-[(6-oxo-1,6-dihydropyridin-3-yl)carbonyl]piperidin-4-yl}-5-(phenylsulfonyl)-2-(trifluoromethyl)benzenesulfonamide.

MS (ES+) m/z 569.8;

HPLC purity 98.4% at 210-370 nm, 7.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1041

### N-[1-(1-acetyl-L-prolyl)piperidin-4-yl]-5-(phenylsulfonyl)-2-(trifluoromethyl)benzenesulfonamide

In an analogous manner to example 1026 step 2, 5-(phenylsulfonyl)-N-piperidin-4-yl-2-(trifluoromethyl)benzenesulfonamide and N-aceyl-L-proline was used to prepare N-[1-(1-acetyl-L-prolyl)piperidin-4-yl]-5-(phenylsulfonyl)-2-(trifluoromethyl)benzenesulfonamide.

MS (ES+) m/z 587.9;

HPLC purity 100.0% at 210-370 nm, 8.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1042

### tert-butyl (5S)-2-oxo-5-{[4-({[5-(phenylsulfonyl)-2-(trifluoromethyl)phenyl]sulfonyl}amino)piperidin-1-yl]carbonyl}pyrrolidine-1-carboxylate

In an analogous manner to example 1026 step 2, 5-(phenylsulfonyl)-N-piperidin-4-yl-2-(trifluoromethyl)benzenesulfonamide and 5-oxo-pyrrolidine-1,2-dicarboxylic acid 1-tert-butyl ester were used to prepare tert-butyl (5S)-2-oxo-5-{[4-({[5-(phenylsulfonyl)-2-(trifluoromethyl)phenyl]sulfonyl}amino)piperidin-1-yl]carbonyl}pyrrolidine-1-carboxylate.

MS (ES−) m/z 658.0;

HPLC purity 96.6% at 210-370 nm, 9.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1043

### N-[1-(5-oxo-L-prolyl)piperidin-4-yl]-5-(phenylsulfonyl)-2-(trifluoromethyl)benzenesulfonamide

In an analogous manner to example 930, tert-butyl (5S)-2-oxo-5-{[4-({[5-(phenylsulfonyl)-2-(trifluoromethyl)phenyl]sulfonyl}amino)piperidin-1-yl]carbonyl}pyrrolidine-1-carboxylate was used to prepare N-[1-(5-oxo-L-prolyl)piperidin-4-yl]-5-(phenylsulfonyl)-2-(trifluoromethyl)benzenesulfonamide.

MS (ES+) m/z 559.8;

HPLC purity 100.0% at 210-370 nm, 7.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1044

### N-(1-hydroxy-6-methoxy-2,3-dihydro-1H-inden-2-yl)-5-(phenylsulfonyl)-2-(trifluoromethyl)benzenesulfonamide

In an analogous manner to example 765, 5-(phenylsulfonyl)-2-(trifluoromethyl)benzenesulfonyl chloride (151.7 mg, 0.5 mmol) and 2-amino-6-methoxy-2,3-dihydro-1H-inden-1-ol (104.4 mg, 0.6 mmol) was used to prepare the title compound N-(1-hydroxy-6-methoxy-2,3-dihydro-1H-inden-2-yl)-5-(phenylsulfonyl)-2-(trifluoromethyl)benzenesulfonamide (171.6 mg, 65%) as a white solid.

HPLC purity 98.0% at 210-370 nm, 9.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₃H₂₀F₃NO₆S₂−H+, 526.06114; found (ESI, [+MH—H2O]+), 510.0851.

### EXAMPLE 1045

### tert-butyl 4-[methyl({[2-({[5-(phenylsulfonyl)-2-(trifluoromethyl)phenyl]sulfonyl}amino)ethyl]amino}carbonyl)amino]piperidine-1-carboxylate

In an analogous manner to Example 887, Step 2, N-2-(aminoethyl)-5-(phenylsulfonyl)-2-(trifluoromethyl)benzenesulfonamide hydrochloride from Example 762 and tert-butyl 4-[(1H-imidazol-1-ylcarbonyl)(methyl)amino]piperidine-1-carboxylat from Example 989, Step1, were used to prepare tert-butyl 4-[methyl({[2-({[5-(phenylsulfonyl)-2-(trifluoromethyl)phenyl]sulfonyl}amino)ethyl]amino}carbonyl)amino]piperidine-1-carboxylate.

MS (ES−) m/z 647.0;

HRMS: calcd for C₂₇H₃₅F₃N₄O₇S₂+H+, 649.19720; found (ESI, [M+H]⁺), 649.1974.

### EXAMPLE 1046

### tert-butyl 4-[({[2-({[5-(phenylsulfonyl)-2-(trifluoromethyl)phenyl]sulfonyl}amino)ethyl]amino}carbonyl)amino]piperidine-1-carboxylate

In an analogous manner to Example 866, Step 2, N-2-(aminoethyl)-5-(phenylsulfonyl)-2-(trifluoromethyl)benzenesulfonamide hydrochloride from Example 762 and tert-butyl 4-[(1H-imidazol-1-ylcarbonyl)amino]piperidine-1-carboxylate from Example 866, Step2, were used to prepare tert-butyl 4-[({[2-({[5-(phenylsulfonyl)-2-(trifluoromethyl)phenyl]sulfonyl}amino)ethyl]amino}carbonyl)amino]piperidine-1-carboxylate.

MS (ESI−) m/z 633;

HRMS: calcd for C₂₆H₃₃F₃N₄O₇S₂+H+, 635.18155; found (ESI, [M+H]⁺), 635.1825.

### EXAMPLE 1047

### N-[1-(1-methyl-L-prolyl)piperidin-4-yl]-5-(phenylsulfonyl)-2-(trifluoromethyl)benzenesulfonamide

In an analogous manner to example 1026 step 2, 5-(phenylsulfonyl)-N-piperidin-4-yl-2-(trifluoromethyl)benzenesulfonamide and N-methyl-L-proline were used to prepare N-[1-(1-methyl-L-prolyl)piperidin-4-yl]-5-(phenylsulfonyl)-2-(trifluoromethyl)benzenesulfonamide.

MS (ES+) m/z 560.2;

HRMS: calcd for C₂₄H₂₈F₃N₃O₅S₂+H+, 560.14952; found (ESI, [M+H]⁺), 560.1518;

HPLC purity 100.0% at 210-370 nm, 7.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1048

### 5-(phenylsulfonyl)-N-[1-(pyridin-3-ylcarbonyl)piperidin-4-yl]-2-(trifluoromethyl)benzenesulfonamide

In an analogous manner to example 462, 5-(phenylsulfonyl)-N-piperidin-4-yl-2-(trifluoromethyl)benzenesulfonamide and pyridine-3-carbonyl chloride were used to prepare 5-(phenylsulfonyl)-N-[1-(pyridin-3-ylcarbonyl)piperidin-4-yl]-2-(trifluoromethyl)benzenesulfonamide.

MS (ES+) m/z 554.0;

HRMS: calcd for C₂₄H₂₂F₃N₃O₅S₂+H+, 554.10257; found (ESI, [M+H]⁺), 554.1022;

HPLC purity 93.8% at 210-370 nm, 8.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1049

### 5-(phenylsulfonyl)-N-[1-(pyridin-2-ylcarbonyl)piperidin-4-yl]-2-(trifluoromethyl)benzenesulfonamide

In an analogous manner to example 462, 5-(phenylsulfonyl)-N-piperidin-4-yl-2-(trifluoromethyl)benzenesulfonamide and pyridine-2-carbonyl chloride were used to prepare 5-(phenylsulfonyl)-N-[1-(pyridin-2-ylcarbonyl)piperidin-4yl]-2-(trifluoromethyl)benzenesulfonamide.

MS (ES) m/z 554.0;

HRMS: calcd for C₂₄H₂₂F₃N₃O₅S₂+H+, 554.10257; found (ESI, [M+H]⁺), 554.1008;

HPLC purity 94.8% at 210-370 nm, 8.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1050

### N-{1-[4-(methylthio)benzoyl]piperidin-4-yl}-5-(phenylsulfonyl)-2-(trifluoromethyl)benzenesulfonamide

In an analogous manner to example1026 step 2, 5-(phenylsulfonyl)-N-piperidin-4-yl-2-(trifluoromethyl)benzenesulfonamide and 4-methylsulfanyl-benzoic acid were used to prepare N-{1-[4-(methylthio)benzoyl]piperidin-4-yl}-5-(phenylsulfonyl)-2-(trifluoromethyl)benzenesulfonamide.

MS (ES+) m/z 598.9;

HRMS: calcd for C₂₆H₂₅F₃N₂O₅S₃+H+, 599.09504; found (ESI, [M+H]⁺), 599.0963;

HPLC purity 100.0% at 210-370 nm, 9.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1051

### 5-(phenylsulfonyl)-2-(trifluoromethyl)-N-(1-([6-(trifluoromethyl)pyridin-3-yl]carbonyl}piperidin-4-yl)benzenesulfonamide

In an analogous manner to example 462, 5-(phenylsulfonyl)-N-piperidin-4-yl-2-(trifluoromethyl)benzenesulfonamide and 6-trifluoromethyl-nicotinoyl chloride were used to prepare 5-(phenylsulfonyl)-2-(trifluoromethyl)-N-(1-{[6-(trifluoromethyl)pyridin-3-yl]carbonyl}piperidin-4-yl)benzenesulfonamide.

MS (ES+) m/z 621.9;

HRMS: calcd for C₂₅H₂₁F₆N₃O₅S₂+H+, 622.08996; found (ESI, [M+H]⁺), 622.0897;

HPLC purity 100.0% at 210-370 nm, 9.5 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1052

### N-{1-[4-(Methylsulfinyl)benzoyl]piperidin-4-yl}-5-(phenylsulfonyl)-2-(trifluoromethyl)benzenesulfonamide

To a stirred solution of N-{1-[4-(methylthio)benzoyl]piperidin-4-yl}-5-(phenylsulfonyl)-2-(trifluoromethyl)benzenesulfonamide (0.08 g, 0.137 mmol) in methylene chloride (1 mL) was added mCPBA (0.31 g 77%, 0.137 mmol) and the resulting mixture was stirred at room temperature for 30 min. The solution was washed with with sodium bicarbonate solution (sat) and concentrated. Flash column separation using 50%-100% ethyl acetate/hexane gradient gave N-{1-[4-(methylsulfinyl)benzoyl]piperidin-4-yl}-5-(phenylsulfonyl)-2-(trifluoromethyl)benzenesulfonamide. (0.058g, 69%).

MS (ES+) m/z 614.9;

HRMS: calcd for C₂₆H₂₅F₃N₂O₆S₃+H+, 615.08996; found (ESI, [M+H]⁺), 615.0925;

HPLC purity 96.3% at 210-370 nm, 8.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1053

### N-{1-[3-(methylthio)propanoyl]piperidin-4-yl}-5-(phenylsulfonyl)-2-(trifluoromethyl)benzenesulfonamide

In an analogous manner to example 1026 step 2, 5-(phenylsulfonyl)-N-piperidin-4-yl-2-(trifluoromethyl)benzenesulfonamide and 3-methylsulfanyl-propionic acid were used to prepare N-{1-[3-(methylthio)propanoyl]piperidin-4-yl}-5-(phenylsulfonyl)-2-(trifluoromethyl)benzenesulfonamide.

HRMS: calcd for C₂₂H₂₅F₃N₂O₅S₃+H+, 551.09504; found (ESI, [M+H]⁺), 551.0927;

HPLC purity 99.1% at 210-370 nm, 9.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1054

### N-{1-[3-(methylsulfinyl)propanoyl]piperidin-4-yl}-5-(phenylsulfonyl)-2-(trifluoromethyl)benzenesulfonamide

In an analogous manner to example 1052, N-{1-[3-(methylthio)propanoyl]piperidin-4-yl}-5-(phenylsulfonyl)-2-(trifluoromethyl)benzenesulfonamide was used to prepare N-{1-[3-(methylsulfinyl)propanoyl]piperidin-4-yl}-5-(phenylsulfonyl)-2-(trifluoromethyl)benzenesulfonamide.

HRMS: calcd for C₂₂H₂₅F₃N₂O₆S₃+H+, 567.08996; found (ESI, [M+H]⁺), 567.0894;

HPLC purity 100.0% at 210-370 nm, 7.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1055

### tert-butyl (4R)-4-{[4-({[5-(phenylsulfonyl)-2-(trifluoromethyl)phenyl]sulfonyl}amino)piperidin-1-yl]carbonyl}-1,3-thiazolidine-3-carboxylate

In an analogous manner to example 1026 step 2, 5-(phenylsulfonyl)-N-piperidin-4-yl-2-(trifluoromethyl)benzenesulfonamide and R-thiazolidine-3,4-dicarboxylic acid 3-tert-butyl ester were used to prepare tert-butyl (4R)-4-{[4-({[5-(phenylsulfonyl)-2-(trifluoromethyl)phenyl]sulfonyl}amino)piperidin-1-yl]carbonyl}-1,3-thiazolidine-3-carboxylate.

MS (ES−) m/z 661.9;

HRMS: calcd for C₂₇H₃₂F₃N₃O₇S₃+H+, 664.14272; found (ESI, [M+H]⁺), 664.1432;

HPLC purity 100.0% at 210-370 nm, 9.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1056

### 5-(phenylsulfonyl)-N-{1-[(4R)-1,3-thiazolidin-4-ylcarbonyl]piperidin-4-yl}-2-(trifluoromethyl)benzenesulfonamide

In an analogous manner to example 930, tert-butyl (4R)-4-{[4-({[5-(phenylsulfonyl)-2-(trifluoromethyl)phenyl]sulfonyl}amino)piperidin-1-yl]carbonyl}-1,3-thiazolidine-3-carboxylate was used to prepare 5-(phenylsulfonyl)-N-{1-[(4R)-1,3-thiazolidin-4-ylcarbonyl]piperidin-4-yl}-2-(trifluoromethyl)benzenesulfonamide.

MS (ES+) m/z 563.8;

HPLC purity 100.0% at 210-370 nm, 8.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1057

### tert-butyl (3R)-3-{(4-({[5-(phenylsulfonyl)-2-(trifluoromethyl)phenyl]sulfonyl}amino)piperidin-1-yl]carbonyl}pyrrolidine-1-carboxylate

In an analogous manner to example 1026 step 2, 5-(phenylsulfonyl)-N-piperidin-4-yl-2-(trifluoromethyl)benzenesulfonamide and R-pyrrolidine-1,3-dicarboxylic acid 1-tert-butyl ester were used to prepare tert-butyl (3R)-3-{[4-({[5-(phenylsulfonyl)-2-(trifluoromethyl)phenyl]sulfonyl}amino)piperidin-1-yl]carbonyl}pyrrolidine-1-carboxylate.

HRMS: calcd for C₂₈H₃₄F₃N₃O₇S₂+H+, 646.18630; found (ESI, [M+H]⁺), 646.189;

HPLC purity 100.0% at 210-370 nm, 9.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1058

### tert-butyl (3S)-3-{[4-({[5-(phenylsulfonyl)-2-(trifluoromethyl)phenyl]sulfonylamino)piperidin-1-yl]carbonyl}pyrrolidine-1-carboxylate

In an analogous manner to example 1026 step 2, 5-(phenylsulfonyl)-N-piperidin-4-yl-2-(trifluoromethyl)benzenesulfonamide and S-pyrrolidine-1,3-dicarboxylic acid 1-tert-butyl ester were used to prepare tert-butyl (3S)-3-{[4-({[5-(phenylsulfonyl)-2-(trifluoromethyl)phenyl]sulfonyl}amino)piperidin-1-yl]carbonyl}pyrrolidine-1-carboxylate.

HRMS: calcd for C₂₈H₃₄F₃N₃O₇S₂+H+, 646.18630; found (ESI, [M+H]⁺), 646.1891;

HPLC purity 100.0% at 210-370 nm, 9.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1059

### 5-(phenylsulfonyl)-N-{1-[(3R)-pyrrolidin-3-ylcarbonyl]piperidin-4-yl}-2-(trifluoromethyl)benzenesulfonamide

In an analogous manner to example 930, tert-butyl (3R)-3-{[4-({[5-(phenylsulfonyl)-2-(trifluoromethyl)phenyl]sulfonyl}amino)piperidin-1-yl]carbonyl}pyrrolidine-1-carboxylate was used to prepare 5-(phenylsulfonyl)-N-{1-[(3R)-pyrrolidin-3-ylcarbonyl]piperidin-4-yl}-2-(trifluoromethyl)benzenesulfonamide.

HRMS: calcd for C₂₃H₂₆F₃N₃O₅S₂+H+, 546.13387; found (ESI, [M+H]⁺), 546.1336;

HPLC purity 95.7% at 210-370 nm, 7.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1060

### 5-(phenylsulfonyl)-N-{1-[(3S)-pyrroldin-3-ylcarbonyl]piperidin-4-yl}-2-(trifluoromethyl)benzenesulfonamide

In an analogous manner to example 930, tert-butyl (3S)-3-{[4-({[5-(phenylsulfonyl)-2-(trifluoromethyl)phenyl]sulfonyl}amino)piperidin-1-yl]carbonyl}pyrrolidine-1-carboxylate was used to prepare 5-(phenylsulfonyl)-N-{1-[(3S)-pyrrolidin-3-ylcarbonyl]piperidin-4-yl}-2-(trifluoromethyl)benzenesulfonamide.

HRMS: calcd for C₂₃H₂₆F₃N₃O₅S₂+H+, 546.13387; found (ESI, [M+H]⁺), 546.1344;

HPLC purity 90.4% at 210-370 nm, 7.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1061

### tert-butyl (4R)-4-{[4-({[5-(phenylsulfonyl)-2-(trifluoromethyl)phenyl]sulfonyl}amino)piperidin-1-yl]carbonyl}-1,3-thiazolidine-3-carboxylate 1-oxide

In an analogous manner to example 1052, tert-butyl (4R)-4-{[4-({[5-(phenylsulfonyl)-2-(trifluoromethyl)phenyl]sulfonyl}amino)piperidin-1-yl]carbonyl}-1,3-thiazolidine-3-carboxylate was used to prepare tert-butyl (4R)-4-{[4-({[5-(phenylsulfonyl)-2-(trifluoromethyl)phenyl]sulfonyl}amino)piperidin-1-yl]carbonyl}-1,3-thiazolidine-3-carboxylate 1-oxide.

MS (ES+) m/z 679.9;

HPLC purity 100.0% at 210-370 nm, 8.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1062

### 2-{[2-({[5-(phenylsulfonyl)-2-(trifluoromethyl)phenyl]sulfonyl}amino)-2,3-dihydro-1H-inden-5-yl]oxy}acetamide

In an analogous manner to example 765, 5-(phenylsulfonyl)-2-(trifluoromethyl)benzenesulfonyl chloride (181.4 mg, 0.6 mmol) and 2-(2-amino-2,3-dihydro-1H-inden-5-yloxy)acetamide (145.7 mg, 0.6 mmol) was used to prepare the title compound 2-{[2-({[5-(phenylsulfonyl)-2-(trifluoromethyl)phenyl]sulfonyl}amino)-2,3-dihydro-1H-inden-5-yl]oxy}acetamide (97.3 mg, 30%) as a yellow solid.

MS (ES+) m/z 554.8;

HPLC purity 95.0% at 210-370 nm, 9.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₄H₂₁F₃N₂O₆S₂+H+, 555.08659; found (ESI, [M+H]⁺), 555.0836.

### EXAMPLE 1063

### methyl [4-({[5-(phenylsulfonyl)-2-(trifluoromethyl)phenyl]sulfonyl)amino)piperidin-1-yl]acetate

In an analogous manner to example 938, [4-({[5-(phenylsulfonyl)-2-(trifluoromethyl)phenyl]sulfonyl}amino)piperidin-1-yl]acetic acid was used to prepare methyl [4-({[5-(phenylsulfonyl)-2-(trifluoromethyl)phenyl]sulfonyl}amino)piperidin-1-yl]acetate.

MS (ES+) m/z 520.8;

HPLC purity 96.3% at 210-370 nm, 6.9 min.; column, Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1064

### N-(1-{[(3R)-1-acetylpyrrolidin-3-yl]carbonyl}piperidin-4-yl)-5-(phenylsulfonyl)-2-(trifluoromethyl)benzenesulfonamide

In an analogous manner to example 462, 5-(phenylsulfonyl)-N-{1-[(3R)-pyrrolidin-3-ylcarbonyl]piperidin-4-yl}-2-(trifluoromethyl)benzenesulfonamide and acyl chloride were used to prepare N-(1-{[(3R)-1-acetylpyrrolidin-3-yl]carbonyl}piperidin-4-yl)-5-(phenylsulfonyl)-2-(trifluoromethyl)benzenesulfonamide.

MS (ES+) m/z 587.8;

HPLC purity 95.1% at 210-370 nm, 8.2 min.; column, Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1065

### N-(1-{[(3S)-1-acetylpyrrolidin-3-yl]carbonyl}piperidin-4-yl)-5-(phenylsulfonyl)-2-(trifluoromethyl)benzenesulfonamide

In an analogous manner to example 462, 5-(phenylsulfonyl)-N-{1-[(3S)-pyrrolidin-3-ylcarbonyl]piperidin-4-yl}-2-(trifluoromethyl)benzenesulfonamide and acyl chloride were used to prepare N-(1-{[(3S)-1-acetylpyrrolidin-3-yl]carbonyl}piperidin-4-yl)-5-(phenylsulfonyl)-2-(trifluoromethyl)benzenesulfonamide.

MS (ES+) m/z 587.8;

HPLC purity 100% at 210-370 nm, 8.2 min.; column, Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1066

### 4-{[3-({[1-(tert-butoxycarbonyl)piperidin-4-yl]amino}sulfonyl)-4-(trifluoromethyl)phenyl]sulfonyl}benzoic acid

Step 1: To a stirred solution of tert-butyl 4-({[5-[(4-fluorophenyl)sulfonyl]-2-(trifluoromethyl)phenyl]sulfonyl}amino)piperidine-1-carboxylate (0.10 g, 0.18 mmol) in DMSO (1 mL) was added sodium cyanide (0.02 g, 0.35 mmol) and the resulting solution was heated to 120° C. overnight. The solution was allowed to cool to room temperature, extracted with ethyl acetate and washed several times with water. The organic phase was concentrated. Flash column separation using 0%-30% ethyl acetate/hexane gradient gave 4-({[5-[(4-cyanophenyl)sulfonyl]-2-(trifluoromethyl)phenyl]sulfonyl}amino)piperidine-1-carboxylate.

Step 2: To a stirred solution of 4-({[5-[(4-cyanophenyl)sulfonyl]-2-(trifluoromethyl)phenyl]sulfonyl)}amino)piperidine-1-carboxylate (0.10 g, 0.17 mmol) in glyme (1 mL) was added 2.5M KOH solution (0.5 mL) and the resulting solution was refluxed overnight. The solution was allowed to cool, neutralized with 2N HCl solution, and extracted with ethyl acetate. The organic phase was concentrated. Flash column separation using 0%-10% methanol/methylene chloride gradient gave 4-{[3-({[1-(tert-butoxycarbonyl)piperidin-4-yl]amino}sulfonyl)-4-(trifluoromethyl)phenyl]sulfonyl}benzoic acid. (0.05 g, 47%).

MS (ES−) m/z 590.8;

HPLC purity 85.3% at 210-370 nm, 9.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1067

### N-[1-(1-isobutyryl-L-prolyl)piperidin-4-yl]-5-(phenylsulfonyl)-2-(trifluoromethyl)benzenesulfonamide

In an analogous manner to example 462, 5-(phenylsulfonyl)-N-(1-L-prolylpiperidin-4-yl)-2-(trifluoromethyl)benzenesulfonamide and isobutyryl chloride were used to prepare N-[1-(1-isobutyryl-L-prolyl)piperidin-4-yl]-5-(phenylsulfonyl)-2-(trifluoromethyl)benzenesulfonamide.

MS (ES+) m/z 615.8;

HPLC purity 100.0% at 210-370 nm, 9.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₇H₃₂F₃N₃O₆S₂+H+, 616.17574; found (ESI, [M+H]⁺), 616.1791.

### EXAMPLE 1068

### N-{1-[1-(2,2-dimethylpropanoyl)-L-prolyl]piperidin-4-yl}-5-(phenylsulfonyl)-2-(trifluoromethyl)benzenesulfonamide

In an analogous manner to example 462,5-(phenylsulfonyl)-N-(1-L-prolylpiperidin-4-yl)-2-(trifluoromethyl)benzenesulfonamide and 2,2-dimethyl-propionyl chloride were used to prepare N-{1-[1-(2,2-dimethylpropanoyl)-L-prolyl]piperidin-4-yl}-5-(phenylsulfonyl)-2-(trifluoromethyl)benzenesulfonamide.

MS (ES+) m/z 629.8;

HPLC purity 95.8% at 210-370 nm, 9.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₈H₃₄F₃N₃O₆S₂+H+, 630.19139; found (ESI, [M+H]⁺), 630.1919.

### EXAMPLE 1069

### N-{1-[1-(3,3-dimethylbutanoyl)-L-prolyl]piperidin-4-yl)-5-(phenylsulfonyl)-2-(trifluoromethyl)benzenesulfonamide

In an analogous manner to example 462, 5-(phenylsulfonyl)-N-(1-L-prolylpiperidin-4-yl)-2-(trifluoromethyl)benzenesulfonamide and 3,3-dimethyl-butyryl chloride were used to prepare N-{1-[1-(3,3-dimethylbutanoyl)-L-prolyl]piperidin-4-yl)-5-(phenylsulfonyl)-2-(trifluoromethyl)benzenesulfonamide.

MS (ES+) m/z 643.9;

HPLC purity 94.6% at 210-370 nm, 9.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1070

### N-{1-[1-(cyclohexylcarbonyl)-L-prolyl]piperidin-4-yl}-5-(phenylsulfonyl)-2-(trifluoromethyl)benzenesulfonamide

In an analogous-manner to example 462,5-(phenylsulfonyl)-N-(1-L-prolylpiperidin-4-yl)-2-(trifluoromethyl)benzenesulfonamide and cyclohexanecarbonyl chloride were used to prepare N-{1-[1-(cyclohexylcarbonyl)-L-prolyl]piperidin-4-yl}-5-(phenylsulfonyl)-2-(trifluoromethyl)benzenesulfonamide.

MS (ES+) m/z 655.9;

HPLC purity 100.0% at 210-370 nm, 8.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1071

### N-{1-[1-(morpholin-4-ylcarbonyl)-L-prolyl]piperidin-4-yl}-5-(phenylsulfonyl)-2-(trifluoromethyl)benzenesulfonamide

In an analogous manner to example 462, 5-(phenylsulfonyl)-N-(1-L-prolylpiperidin-4-yl)-2-(trifluoromethyl)benzenesulfonamide and morpholine-4-carbonyl chloride were used to prepare N-{1-[1-(morpholin-4-ylcarbonyl)-L-prolyl]piperidin-4-yl}-5-(phenylsulfonyl)-2-(trifluoromethyl)benzenesulfonamide.

MS (ES+) m/z 658.8;

HPLC purity 100.0% at 210-370 nm, 9.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₈H₃₅F₃N₄O₆S₂+H+, 645.20229; found (ESI, [M+H]⁺), 645.2024.

### EXAMPLE 1072

### (2S)-N-(tert-butyl)-2-{[4-({[5-(phenylsulfonyl)-2-(trifluoromethyl)phenyl]sulfonyl}amino)piperidin-1-yl]carbonyl}pyrrolidine-1-carboxamide

In an analogous manner to example 462, 5-(phenylsulfonyl)-N-(1-L-prolylpiperidin-4-yl)-2-(trifluoromethyl)benzenesulfonamide and tert-butylisocyanate were used to prepare (2S)-N-(tert-butyl)-2-{[4-({[5-(phenylsulfonyl)-2-(trifluoromethyl)phenyl]sulfonyl}amino)piperidin-1-yl]carbonyl}pyrrolidine-1-carboxamide.

MS (ES+) m/z 644.9;

HPLC purity 100.0% at 210-370 nm, 10.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₂₈H₃₅F₃N₄O₆S₂+H+, 645.20229; found (ESI, [M+H]⁺), 645.2048.

### EXAMPLE 1074

### (2S)-N-phenyl-2-{[4-({[5-(phenylsulfonyl)-2-(trifluoromethyl)phenyl]sulfonyl}amino)piperidin-1-yl]carbonyl}pyrrolidine-1-carboxamide

In an analogous manner to example 462, 5-(phenylsulfonyl)-N-(1-L-prolylpiperidin-4-yl)-2-(trifluoromethyl)benzenesulfonamide and phenylisocyanate were used to prepare (2S)-N-phenyl-2-{[4-({[5-(phenylsulfonyl)-2-(trifluoromethyl)phenyl]sulfonyl}amino)piperidin-1-yl]carbonyl}pyrrolidine-1-carboxamide.

MS (ES+) m/z 664.8;

HPLC purity 100.0% at 210-370 nm, 9.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1075

### N-{1-[1-(methylsulfonyl)-L-prolyl]piperidin-4-yl}-5-(phenylsulfonyl)-2-(trifluoromethyl)benzenesulfonamide

In an analogous manner to example 462, 5-(phenylsulfonyl)-N-(1-L-prolylpiperidin-4-yl)-2-(trifluoromethyl)benzenesulfonamide and methane sulfonyl chloride were used to prepare N-{1-[1-(methylsulfonyl)-L-prolyl]piperidin-4-yl)}-5-(phenylsulfonyl)-2-(trifluoromethyl)benzenesulfonamide.

MS (ES+) m/z 623.8;

HRMS: calcd for C₂₄H₂₈F₃N₃O₇S₃+H+, 624.11142; found (ESI, [M+H]⁺), 624.1116;

HPLC purity 100.0% at 210-370 nm, 8.5 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1076

### N-[1-(1-benzoyl-L-prolyl)piperidin-4-yl]-5-(phenylsulfonyl)-2-(trifluoromethyl)benzenesulfonamide

In an analogous manner to example 462, 5-(phenylsulfonyl)-N-(1-L-prolylpiperidin-4-yl)-2-(trifluoromethyl)benzenesulfonamide and benzoyl chloride were used to prepare N-[1-(1-benzoyl-L-prolyl)piperidin-4-yl]-5-(phenylsulfonyl)-2-(trifluoromethyl)benzenesulfonamide.

MS (ES+) m/z 649.8;

HPLC purity 94.7% at 210-370 nm, 9.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₃₀H₃₀F₃N₃O₆S₂+H+, 650.16009; found (ESI, [M+H]⁺), 650.1616.

### EXAMPLE 1077

### N-(1-{1-[4-(dimethylamino)benzoyl]-L-prolyl}piperidin-4-yl)-5-(phenylsulfonyl)-2-(trifluoromethyl)benzenesulfonamide

In an analogous manner to example 462, 5-(phenylsulfonyl)-N-(1-L-prolylpiperidin-4-yl)-2-(trifluoromethyl)benzenesulfonamide and 4-demethylaminobenzoyl chloride were used to prepare N-(1-{1-[4-(dimethylamino)benzoyl]-L-prolyl}piperidin-4-yl)-5-(phenylsulfonyl)-2-(trifluoromethyl)benzenesulfonamide.

MS (ES+) m/z 692.9;

HPLC purity 94.2% at 210-370 nm, 9.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₃₂H₃₅F₃N₄O₆S₂+H+, 693.20229; found (ESI, [M+H]⁺), 693.2034.

### EXAMPLE 1078

### N-[1-(1-isonicotinoyl-L-prolyl)piperidin-4-yl]-5-(phenylsulfonyl)-2-(trifluoromethyl)benzenesulfonamide

In an analogous manner to example 462, 5-(phenylsulfonyl)-N-(1-L-prolylpiperidin-4-yl)-2-(trifluoromethyl)benzenesulfonamide and isonicotinoyl chloride were used to prepare N-[1-(1-isonicotinoyl-L-prolyl)piperidin-4-yl]-5-(phenylsulfonyl)-2-(trifluoromethyl)benzenesulfonamide.

MS (ES+) m/z 650.8;

HRMS: calcd for C₂₉H₂₉F₃N₄O₆S₂+H+, 651.15534; found (ESI, [M+H]⁺), 651.1552;

HPLC purity 100.0% at 210-370 nm, 9.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1079

### N-(1-{1-[(6-chloropyridin-3-yl)carbonyl]-L-prolyl}piperidin-4-yl)-5-(phenylsulfonyl)-2-(trifluoromethyl)benzenesulfonamide

In an analogous manner to example 462, 5-(phenylsulfonyl)-N-(1-L-prolylpiperidin-4-yl)-2-(trifluoromethyl)benzenesulfonamide and 6-chloro-nicotinoyl chloride were used to prepare N-(1-{1-[(6-chloropyridin-3-yl)carbonyl]-L-prolyl}piperidin-4-yl)-5-(phenylsulfonyl)-2-(trifluoromethyl)benzenesulfonamide.

MS (ES−) m/z 684.8;

HPLC purity 100.0% at 210-370 nm, 9.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1080

### 4-[((2S)-2-{[4-({[5-(phenylsulfonyl)-2-(trifluoromethyl)phenyl]sulfonyl}amino)piperidin-1-yl]carbonyl}pyrrolidin-1-yl)sulfonyl]benzoic acid

In an analogous manner to example 462, 5-(phenylsulfonyl)-N-(1-L-prolylpiperidin-4-yl)-2-(trifluoromethyl)benzenesulfonamide and 4-(chlorosulfonyl)benzoic acid were used to prepare 4-[((2S)-2-{[4-({[5-(phenylsulfonyl)-2-(trifluoromethyl)phenyl]sulfonyl}amino)piperidin-1-yl]carbonyl}pyrrolidin-1-yl)sulfonyl]benzoic acid.

MS (ES+) m/z 729.8;

HPLC purity 100.0% at 210-370 nm, 8.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1081

### N-[1-(N,N-dimethylglycyl-L-prolyl)piperidin-4-yl]-5-(phenylsulfonyl)-2-(trifluoromethyl)benzenesulfonamide

In an analogous manner to example 462, 5-(phenylsulfonyl)-N-(1-L-prolylpiperidin-4-yl)-2-(trifluoromethyl)benzenesulfonamide and dimethylamino-acetyl chloride were used to prepare N-[1-(N,N-dimethylglycyl-L-prolyl)piperidin-4-yl]-5-(phenylsulfonyl)-2-(trifluoromethyl)benzenesulfonamide.

MS (ES+) m/z 630.9;

HPLC purity 87.6% at 210-370 nm, 7.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1082

### 2-({[5-(phenylsulfonyl)-2-(trifluoromethyl)phenyl]sulfonyl}amino)indane-5-carboxylic acid

Methyl 2-({[5-(phenylsulfonyl)-2-(trifluoromethyl)phenyl]sulfonyl}amino)indane-5-carboxylate (478.2 mg, 0.9 mmol) prepare in example 1037 was taken up in 25 ml of a solution of 2:2:1 tetrahydrofuran:methanol:water. To the stirring solution 2N sodium hydroxide (880 μL, 1.76 mmol) was syringed into the reaction flask and allowed to stir overnight at room temperature. 2N Hydrochloric acid (900 μL, 1.8 mmol) was syringed into the reaction flask that caused a white solid to crash out of solution. The solid was collected by filtration and the product was transferred onto a 12 g Isco RediSep® Normal Phase column and was purified by automated flash chromatography using a gradient of 20% to 100% hexane/ethyl acetate. Isolation of the main component gave the title compound 2-({[5-(phenylsulfonyl)-2-(trifluoromethyl)phenyl]sulfonyl}amino)indane-5-carboxylic acid (122.2 mg, 26%) as a white solid.

MS (ES−) m/z 525.7;

HPLC purity 97.2% at 210-370 nm, 9.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1083

### N-methyl-2-[4-({[5-(phenylsulfonyl)-2-(trifluoromethyl)phenyl]sulfonyl}amino)piperidin-1-yl]acetamide

In an analogous manner to example 880,5-(phenylsulfonyl)-N-piperidin-4-yl-2-(trifluoromethyl)benzenesulfonamide and 2-chloro-N-methyl-acetamide were used to prepare N-methyl-2-[4-({[5-(phenylsulfonyl)-2-(trifluoromethyl)phenyl]sulfonyl}amino)piperidin-1-yl]acetamide.

MS (ES+) m/z 519.8;

HPLC purity 100.0% at 210-370 nm, 7.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1084

### N-[1-(1-benzyl-L-prolyl)piperidin-4-yl]-5-(phenylsulfonyl)-2-(trifluoromethyl)benzenesulfonamide

In an analogous manner to example 880, 5-(phenylsulfonyl)-N-(1-L-prolylpiperidin-4-yl)-2-(trifluoromethyl)benzenesulfonamide and benzyl bromide were used to prepare N-[1-(1-benzyl-L-prolyl)piperidin-4-yl]-5-(phenylsulfonyl)-2-(trifluoromethyl)benzenesulfonamide.

MS (ES+) m/z 635.9;

HPLC purity 100.0% at 210-370 nm, 8.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1085

### N,N-dimethyl-2-[4-({[5-(phenylsulfonyl)-2-(trifluoromethyl)phenyl]sulfonyl}amino)piperidin-1-yl]acetamide

In an analogous manner to example 880, 5-(phenylsulfonyl)-N-piperidin-4-yl-2-(trifluoromethyl)benzenesulfonamide and 2-chloro-N,N-dimethyl-acetamide were used to prepare N,N-dimethyl-2-[4-({[5-(phenylsulfonyl)-2-(trifluoromethyl)phenyl]sulfonyl}amino)piperidin-1-yl]acetamide.

MS (ES+) m/z 533.9;

HPLC purity 96.6% at 210-370 nm, 6.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1086

### N-isopropyl-2-[4-({[5-(phenylsulfonyl)-2-(trifluoromethyl)phenyl]sulfonyl}amino)piperidin-1-yl]acetamide

In an analogous manner to example 880, 5-(phenylsulfonyl)-N-piperidin-4-yl-2-(trifluoromethyl)benzenesulfonamide and 2-chloro-N-isopropyl-acetamide were used to prepare N-isopropyl-2-[4-({[5-(phenylsulfonyl)-2-(trifluoromethyl)phenyl]sulfonyl}amino)piperidin-1-yl]acetamide.

MS (ES−) m/z 547.8;

HPLC purity 95.1% at 210-370 nm, 7.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1087

### N-[1-(2-morpholin-4-yl-2-oxoethyl)piperidin-4-yl]-5-(phenylsulfonyl)-2-(trifluoromethyl)benzenesulfonamide

In an analogous manner to example 880, 5-(phenylsulfonyl)-N-piperidin-4-yl-2-(trifluoromethyl)benzenesulfonamide and 2-chloro-1-morpholin-4-yl-ethanone were used to prepare N-[1-(2-morpholin-4-yl-2-oxoethyl)piperidin-4-yl]-5-(phenylsulfonyl)-2-(trifluoromethyl)benzenesulfonamide.

MS (ES+) m/z 575.8;

HPLC purity 98.2% at 210-370 nm, 7.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1088

### N-{1-[1-(cyclohexylmethyl)-L-prolyl]piperidin-4-yl}-5-(phenylsulfonyl)-2-(trifluoromethyl)benzenesulfonamide

To a stirred solution of 5-(phenylsulfonyl)-N-(1-L-prolylpiperidin-4-yl)-2-(trifluoromethyl)benzenesulfonamide (0.09 g, 0.16 mmol) and cyclohexanecarbaldehyde (0.02 g, 0.16 mmol) in methanol (1 mL) was added triacetoxy sodium borohydride (0.05 g, 0.22 mmol) and the resulting solution was stirred overnight at room temperature. The crude mixture was concentrated. Flash column separation using 0%-10% methanol/methylene chloride gradient gave N-{1-[1-(cyclohexylmethyl)-L-prolyl]piperidin-4-yl}-5-(phenylsulfonyl)-2-(trifluoromethyl)benzenesulfonamide. (0.025 g, 25%).

MS (ES+) m/z 641.9;

HPLC purity 100.0% at 210-370 nm, 8.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1089

### N-{1-[1-(3,3-dimethylbutyl)-L-prolyl]piperidin-4-yl)}-5-(phenylsulfonyl)-2-(trifluoromethyl)benzenesulfonamide

In an analogous manner to example 1088, 5-(phenylsulfonyl)-N-(1-L-prolylpiperidin-4-yl)-2-(trifluoromethyl)benzenesulfonamide and 3,3-dimethyl-butyraldehyde were used to prepare N-{1-[1-(3,3-dimethylbutyl)-L-prolyl]piperidin-4-yl}-5-(phenylsulfonyl)-2-(trifluoromethyl)benzenesulfonamide.

MS (ES+) m/z 630.0;

HPLC purity 100.0% at 210-370 nm, 8.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1090

### 5-(phenylsulfonyl)-N-[1-(1,3-thiazol-4-ylcarbonyl)piperidin-4-yl]-2-(trifluoromethyl)benzenesulfonamide

In an analogous manner to example 1026 step 2, 5-(phenylsulfonyl)-N-piperidin-4-yl-2-(trifluoromethyl)benzenesulfonamide and thiazole-4-carboxylic acid were used to prepare 5-(phenylsulfonyl)-N-[1-(1,3-thiazol-4-ylcarbonyl)piperidin-4-yl]-2-(trifluoromethyl)benzenesulfonamide.

MS (ES+) m/z 559.6;

HPLC purity 98.5% at 210-370 mn, 8.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1091

### (2S)—N-ethyl-2-{[4-({[5-(phenylsulfonyl)-2-(trifluoromethyl)phenyl]sulfonyl}amino)piperidin-1-yl]carbonyl}pyrrolidine-1-carboxamide

In an analogous manner to example 462, 5-(phenylsulfonyl)-N-(1-L-prolylpiperidin-4-yl)-2-(trifluoromethyl)benzenesulfonamide and ethylisocyanate were used to prepare (2S)—N-ethyl-2-{[4-({[5-(phenylsulfonyl)-2-(trifluoromethyl)phenyl]sulfonyl}amino)piperidin-1-yl]carbonyl}pyrrolidine-1-carboxamide.

MS (ES+) m/z 616.8;

HPLC purity 100.0% at 210-370 nm, 8.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1092

### (2S)—N,N-dimethyl-2-{[4-({[5-(phenylsulfonyl)-2-(trifluoromethyl)phenyl]sulfonyl}amino)piperidin-1-yl]carbonyl}pyrrolidine-1-carboxamide.

In an analogous manner to example 462, 5-(phenylsulfonyl)-N-(1-L-prolylpiperidin-4-yl)-2-(trifluoromethyl)benzenesulfonamide and dimethylaminocarbonyl chloride were used to prepare (2S)—N,N-dimethyl-2-{[4-({[5-(phenylsulfonyl)-2-(trifluoromethyl)phenyl]sulfonyl}amino)piperidin-1-yl]carbonyl}pyrrolidine-1-carboxamide.

MS (ES+) m/z 616.8;

HPLC purity 100.0% at 210-370 nm, 8.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1093

### N-[1-(1H-imidazol-1-ylacetyl)piperidin-4-yl]-5-(phenylsulfonyl)-2-(trifluoromethyl)benzenesulfonamide

In an analogous manner to example 1026 step 2, 5-(phenylsulfonyl)-N-piperidin-4-yl-2-(trifluoromethyl)benzenesulfonamide and imidazol-1-yl-acetic acid were used to prepare N-[1-(1H-imidazol-1-ylacetyl)piperidin-4-yl]-5-(phenylsulfonyl)-2-(trifluoromethyl)benzenesulfonamide.

MS (ES+) m/z 556.8;

HPLC purity 99.2% at 210-370 nm, 7.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1094

### N-(5-bromo-2,3-dihydro-1H-inden-2-yl)-5-(phenylsulfonyl)-2-(trifluoromethyl)benzenesulfonamide

To a stirred suspension of 5-bromoindan-2-amine hydrobromide (2.80 g, 9.56 mmol) in a solution of 5-(phenylsulfonyl)-2-(trifluoromethyl)benzenesulfonyl chloride (3.67 g, 9.54 mmol) from Example 677 in CH₂Cl₂ (75 mL) under N₂ at room temperature was added diisopropylethylamine (5.00 mL, 3.71 g, 28.7 mmol). The mixture was stirred at room temperature for 1.5 hours. It was washed once with 2N HCl, twice with water and once with brine. The solvent was evaporated. The residue was dissolved again in CH₂Cl₂ (18 mL) The solution was loaded directly onto a silica gel column and eluted with a gradient of hexane and CH₂Cl₂ to give 4.11 g (77%) of N-(5-bromo-2,3-dihydro-1H-inden-2-yl)-5-(phenylsulfonyl)-2-(trifluoromethyl)benzenesulfonamide.

MS (ES−) m/z 557.6.

### EXAMPLE 1095

### tert-butyl (2S)-2-{[4-({[5-[(3-cyanophenyl)sulfonyl]-2-(trifluoromethyl)phenyl]sulfonyl}amino)piperidin-1-yl]carbonyl}pyrrolidine-1-carboxylate

Step 1: In an analogous manner to example 1026 step 2, 5-[(3-bromophenyl)sulfonyl]-N-piperidin-4-yl-2-(trifluoromethyl)benzenesulfonamide and N-(tert-butoxycarbonyl)-L-proline were used to prepare tert-butyl (2S)-2-{[4-({[5-[(3-bromophenyl)sulfonyl]-2-(trifluoromethyl)phenyl]sulfonyl}amino)piperidin-1-yl]carbonyl}pyrrolidine-1-carboxylate.

Step 2: In an analogous manner to example 376, tert-butyl (2S)-2-{[4-({[5-[(3-bromophenyl)sulfonyl]-2-(trifluoromethyl)phenyl]sulfonyl}amino)piperidin-1-yl]carbonyl}pyrrolidine-1-carboxylate was used to prepare tert-butyl (2S)-2-{[4-({[5-[(3-cyanophenyl)sulfonyl]-2-(trifluoromethyl)phenyl]sulfonyl}amino)piperidin-1-yl]carbonyl}pyrrolidine-1-carboxylate.

MS (ES+) m/z 670.7

HPLC purity 98.8% at 210-370 nm, 11.2 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### EXAMPLE 1096

### Fluorescence Polarization Binding Assay

The affinity of test compounds for SFRP-1 was determined using a fluorescence polarization binding assay. According to the assay design, a probe compound was bound to SFRP-1. The fluorescence anisotropy value of the probe compound is increased upon binding to SFRP-1. Upon the addition of a test compound, the fluorescence anisotropy value for the probe compound decreased due to competitive displacement of the probe by the test compound. The decrease in anisotropy as a function of increasing concentration of the test compound provides a direct measure of the test compound's binding affinity for SFRP-1.

To determine IC50 values, fluorescence polarization experiments were conducted in a 384-well format according to the following procedures. A 20 mM stock solution of the probe compound was prepared in 100% DMSO and dispensed in 10 μL aliquots for long-term storage at −20° C. The binding assay buffer was prepared by combining stock solutions of Tris-Cl, NacL, glycerol, and NP40 at final concentrations of 25 mM Tris-Cl pH 7.4, 0.5 M NaCl, 5% glycerol and 0.002% NP40. Master stock solutions of the test compounds were prepared in 100% DMSO at final concentrations of 20 mM. Typically the working stock solutions of the test compounds were prepared by serially diluting the 20 mM master stock solution to 5 mM, 2.5 mM, 1.25 mM, 0.625 mM, 0.3125 mM, 0.156 mM, 78 μM, 39 μM, 19.5 M, 9.8 μM, 4.9 μM, 2.44 μM, 1.22 μM, 0.31 μM, 76 nM, and 19 nM in DMSO. The working stock solutions of the test compounds were further diluted by combining 6 μl of the solutions with 24 μL of Milli-Q purity water, resulting in working stock solutions (10× compound stocks) in 20% DMSO.

The assay controls were prepared as follows. A 2 μL aliquot of the 20 mM fluorescence probe compound was diluted 1000-fold in 100% DMSO to a final concentration of 20 μM. 6 μL of the 20 μM probe was combined with 5.4 mL of the assay buffer, mixed well, and 18 μL of the resulting solution was dispensed into 384-well plates.

SFRP-1/probe complex was prepared by combining 11 μL of 20 M probe compound with 9.9 mL of the assay buffer and SFRP-1 stock solution to final concentrations of 22 nM probe compound and 50 nM SFRP-1. 18 μL of the SFRP-1/probe complex was dispensed into the 384-well plates.

2 μL aliquots of the test compounds from the 10× working stock solutions were removed and dispensed into the plate containing the SFRP-1/probe complex and the resultant solutions were mixed by pipetting up and down once. The final concentratons of SFRP-1 and probe in the assay solutions were 45 nM and 20 nM, respectively. In a typical experiment, each plate was used to test 14 compounds.

The plate was incubated in the dark for 15 minutes. The fluorescence of the SFRP-1/probe complexs was read in the Tecan Ultra plate reader at excitation and emission maxima of 485 and 535 nm. The plate reader settings were as follows:

Mode: Fluorescence Polarization

Plate definition: Matrical3841v.pdf

(pdf stands for Plate Definition File)

Excitation 485 nm (bandwidth 20 nm)

Emission 535 nm (bandwidth 30 nm)

G-factor: 1.03

# flashes/well: 10

integration time: 100 us

time btwn move, flash: 60 ms

Z-position: 10730 um

Analysis of Results

Fluorescence anisotropy results from the emission of polarized light in the parallel and perpendicular directions when a fluorophore is excited with vertically polarized light. The anisotropy of the probe in the free and bound state was determined using the following equation:
r=I(II)−I(⊥)÷I(II)+2I(⊥)
where I(II) and I(I) are the parallel and perpendicular emission intensities, respectively.

Monitoring the anisotropy changes of the probe compound revealed that it bound saturably to SFRP-1 with a K_{D} of 20-30 nM. The binding affinity was independently verified using a tryptophan fluorescence quenching assay.

The decrease in the anisotropy of the probe upon addition of the competing test compound was fitted to a sigmoidal dose response curve of the equation shown below:

$$Y = Bottom + \frac{( Top - Bottom )}{( 1 + {10}^{X - Log IC 50} )} * Hillslope$$

where “X” is the logarithm of concentration, “Y” is the anisotropy, and “Bottom” and “Top” correspond to the anisotropy values of the free and SFRP-1-bound probe prior to the addition of the test compound, respectively.

For automated IC50 determinations, the equation shown above was used in the program GraphPad Prism. The “Hillslope” was kept constant at 1. The value for “Bottom” was fixed, but was determined by the blank (probe-only) wells in the plate. The values for “Top” and “IC50” were determined by the data fit. The value for “Top” was typically close to 120, equivalent to approximately 50% bound probe, and the value for “Bottom” was around 30, due to free probe. If the test compound interfered with the probe in the fluorescence assay at high concentrations, the range for the fitted data was limited to the lower concentration range. The data obtained from the experiments are shown in the table below.

| Example | IC50 (μM) | INHIBITION (%) | CONC (μg/ml) |
|---|---|---|---|
| 163 | | 71 | 45.7 |
| 169 | 13 | | |
| 173 | | 78 | 39 |
| 175 | | 77 | 39 |
| 177 | 2.9 | | |
| 203 | | 84 | 38.2 |
| 203 | | 64 | 38.3 |
| 204 | 8 | | |
| 205 | 3 | | |
| 206 | | 75 | 43.9 |
| 219 | 3 | | |
| 219 | 1.1 | | |
| 219 | 2.1 | | |
| 223 | 0.9 | | |
| 223 | 0.9 | | |
| 226 | 2.6 | | |
| 226 | 2.6 | | |
| 227 | 4 | | |
| 228 | 8 | | |
| 228 | 15 | | |
| 230 | 16 | | |
| 231 | 0.3 | | |
| 231 | 0.6 | | |
| 234 | | 29 | 43.3 |
| 235 | | 49 | 43.3 |
| 242 | | 58 | 43.6 |
| 295 | 0.3 | | |
| 296 | 0.2 | | |
| 297 | 0.4 | | |
| 298 | 0.8 | | |
| 303 | 0.7 | | |
| 310 | 0.4 | | |
| 311 | 0.4 | | |
| 313 | 0.5 | | |
| 314 | 1.2 | | |
| 318 | 0.4 | | |
| 318 | 0.5 | | |
| 318 | 0.6 | | |
| 318 | 0.7 | | |
| 318 | 0.9 | | |
| 319 | 0.6 | | |
| 320 | 0.9 | | |
| 321 | 1 | | |
| 322 | 0.6 | | |
| 324 | 0.9 | | |
| 328 | 3 | | |
| 329 | 1 | | |
| 344 | 1.4 | | |
| 353 | 0.3 | | |
| 354 | 1.4 | | |
| 356 | 1.7 | | |
| 357 | 0.3 | | |
| 358 | 0.6 | | |
| 358 | 0.6 | | |
| 359 | 0.8 | | |
| 360 | 2 | | |
| 368 | 0.9 | | |
| 370 | 0.5 | | |
| 370 | 0.5 | | |
| 371 | 7 | 22 | 3.4 |
| 372 | 2 | | |
| 372 | 2.1 | | |
| 373 | 1.1 | | |
| 381 | 4 | | |
| 390 | | 67 | 44.56 |
| 392 | 4 | | |
| 398 | 9 | | |
| 399 | 3 | | |
| 402 | 2.6 | | |
| 413 | 5 | | |
| 414 | | 70 | 23.53 |
| 418 | 1.3 | | |
| 420 | 8 | | |
| 421 | 9 | | |
| 422 | 2.8 | | |
| 423 | 10 | | |
| 424 | | 71 | 35.14 |
| 425 | 2.8 | | |
| 427 | 1.1 | | |
| 427 | 1.1 | | |
| 435 | 2.6 | | |
| 436 | 1 | | |
| 437 | 5 | | |
| 439 | 2 | | |
| 440 | 3 | | |
| 441 | 6 | | |
| 442 | 1 | | |
| 443 | 0.6 | | |
| 445 | 1.1 | | |
| 445 | 1.1 | | |
| 446 | 2 | | |
| 448 | 0.12 | | |
| 448 | 0.17 | | |
| 449 | 3 | | |
| 450 | 0.4 | | |
| 450 | 0.2 | | |
| 450 | 0.3 | | |
| 452 | 0.2 | | |
| 453 | 0.3 | | |
| 455 | 0.5 | | |
| 456 | 0.8 | | |
| 457 | 1.1 | | |
| 461 | 0.7 | | |
| 462 | 2 | | |
| 463 | 0.7 | | |
| 464 | 3 | | |
| 465 | 4 | | |
| 466 | 1.7 | | |
| 467 | 1.9 | | |
| 468 | 1 | | |
| 471 | 2.2 | | |
| 474 | 0.8 | | |
| 476 | 1.4 | | |
| 477 | 2.2 | | |
| 478 | 1.3 | | |
| 480 | 2.9 | | |
| 481 | 1.2 | | |
| 482 | 0.8 | | |
| 484 | 1.5 | | |
| 485 | 1.7 | | |
| 486 | 5 | | |
| 487 | 0.5 | | |
| 488 | 2.1 | | |
| 489 | 1 | | |
| 490 | 2.3 | | |
| 496 | 1.4 | | |
| 497 | 1.3 | | |
| 497 | 1.7 | | |
| 498 | 2.2 | | |
| 499 | 0.9 | | |
| 499 | 0.9 | | |
| 501 | 4 | | |
| 501 | 4 | | |
| 502 | 3 | | |
| 503 | 2.4 | | |
| 504 | 4 | | |
| 505 | 0.7 | | |
| 506 | 2.4 | | |
| 507 | 0.5 | | |
| 508 | 1.3 | | |
| 509 | 1.2 | | |
| 510 | 0.9 | | |
| 511 | 1.8 | | |
| 511 | 2.3 | | |
| 512 | 1 | | |
| 513 | 6 | | |
| 514 | 0.7 | | |
| 516 | 2.6 | | |
| 517 | 1.6 | | |
| 517 | 1.6 | | |
| 518 | 0.5 | | |
| 519 | 0.8 | | |
| 519 | 0.8 | | |
| 520 | 2.4 | | |
| 523 | 3 | | |
| 525 | 1 | | |
| 526 | 0.5 | | |
| 528 | 0.3 | | |
| 529 | 0.5 | | |
| 530 | 0.7 | | |
| 531 | 0.4 | | |
| 532 | 4 | | |
| 533 | 0.5 | | |
| 534 | 0.2 | | |
| 534 | 0.3 | | |
| 535 | 0.4 | | |
| 536 | 0.4 | | |
| 537 | 0.7 | | |
| 539 | 2.6 | | |
| 540 | 0.2 | | |
| 541 | 1.5 | | |
| 542 | 1.4 | | |
| 543 | 0.8 | | |
| 543 | 1.6 | | |
| 544 | 0.8 | | |
| 544 | 1.7 | | |
| 544 | 0.8 | | |
| 545 | 2 | | |
| 545 | 4 | | |
| 546 | 1.5 | | |
| 546 | 2.6 | | |
| 547 | 1 | | |
| 547 | 2.5 | | |
| 548 | 2.2 | | |
| 549 | 1.6 | | |
| 549 | 2.9 | | |
| 550 | 1.3 | | |
| 551 | 1 | | |
| 552 | 1.2 | | |
| 553 | 3 | | |
| 554 | 1.7 | | |
| 555 | 0.7 | | |
| 555 | 1.1 | | |
| 556 | 0.3 | | |
| 558 | 0.7 | | |
| 559 | 1.2 | | |
| 560 | 1.7 | | |
| 560 | 2.7 | | |
| 561 | 1.4 | | |
| 562 | 1.6 | | |
| 563 | 3 | | |
| 564 | 1.4 | | |
| 564 | 1.6 | | |
| 565 | 0.5 | | |
| 567 | 1.6 | | |
| 568 | 0.5 | | |
| 569 | 1.5 | | |
| 570 | 0.16 | | |
| 571 | 0.13 | | |
| 571 | 0.3 | | |
| 571 | 0.3 | | |
| 572 | 0.5 | | |
| 573 | 0.6 | | |
| 574 | 6 | | |
| 575 | 1.8 | | |
| 577 | 0.7 | | |
| 578 | 0.6 | | |
| 579 | 0.6 | | |
| 580 | 0.7 | | |
| 581 | 1 | | |
| 583 | 0.3 | | |
| 584 | 0.5 | | |
| 585 | 8 | | |
| 586 | 2.4 | | |
| 587 | 5 | | |
| 588 | 0.4 | | |
| 589 | 4 | | |
| 590 | 2 | | |
| 590 | 2.5 | | |
| 591 | 5 | | |
| 591 | 6 | | |
| 592 | 1 | | |
| 593 | 0.9 | | |
| 594 | 4 | | |
| 595 | 1.2 | | |
| 596 | 1.6 | | |
| 597 | 0.4 | | |
| 598 | 0.9 | | |
| 599 | 0.6 | | |
| 600 | 2 | | |
| 602 | | 10 | 13.73 |
| 603 | 8 | | |
| 604 | 7 | | |
| 605 | 0.6 | | |
| 606 | 8 | | |
| 607 | 2 | | |
| 608 | 0.3 | | |
| 608 | 0.5 | | |
| 609 | 2.1 | | |
| 610 | 0.9 | | |
| 611 | 1.3 | | |
| 612 | 1.6 | | |
| 613 | 2.5 | | |
| 613 | 3 | | |
| 614 | 1.3 | | |
| 615 | 1.2 | | |
| 616 | 0.8 | | |
| 617 | 1 | | |
| 620 | 1 | | |
| 620 | 1 | | |
| 621 | | 10 | 11.69 |
| 623 | | 20 | 6.44 |
| 625 | 3 | | |
| 625 | 3 | | |
| 626 | 4 | | |
| 626 | 4 | | |
| 627 | 5 | | |
| 627 | 5 | | |
| 628 | 3 | | |
| 628 | 3 | | |
| 629 | 3 | | |
| 629 | 3 | | |
| 630 | 4 | | |
| 630 | 4 | | |
| 631 | 4 | | |
| 631 | 4 | | |
| 632 | 2.6 | | |
| 632 | 2.6 | | |
| 635 | | 40 | 46.9 |
| 636 | | 57 | 25.7 |
| 637 | | 10 | 6.69 |
| 639 | | 10 | 12.88 |
| 640 | 1.2 | | |
| 641 | 1.1 | | |
| 642 | | 45 | 47.2 |
| 643 | 2.4 | | |
| 644 | | 49 | 22.85 |
| 646 | | 20 | 45.1 |
| 647 | 1.4 | | |
| 648 | 5 | | |
| 649 | 1.6 | | |
| 650 | 10 | | |
| 651 | 0.4 | | |
| 652 | 0.8 | | |
| 652 | 0.7 | | |
| 653 | | 10 | 6.34 |
| 654 | | 10 | 43.19 |
| 655 | | 36 | 40 |
| 656 | 2.8 | | |
| 657 | 2.4 | | |
| 658 | 0.8 | | |
| 659 | | | |
| 660 | 2.4 | | |
| 662 | 4 | | |
| 663 | | 10 | 18.5 |
| 664 | 1.7 | | |
| 665 | | 73 | 21.8 |
| 666 | | 71 | 21.9 |
| 667 | | 42 | 6.4 |
| 668 | | 48 | 11.8 |
| 669 | | 33 | 6.4 |
| 670 | 0.8 | | |
| 671 | 0.4 | | |
| 672 | 0.3 | | |
| 673 | | 81 | 37.6 |
| 674 | | 77 | 39 |
| 675 | | 57 | 40.6 |
| 676 | 9 | | |
| 677 | 0.16 | | |
| 678 | | 49 | 21.6 |
| 679 | 0.25 | | |
| 680 | 0.4 | | |
| 681 | 1.1 | | |
| 682 | 0.25 | | |
| 683 | 1.2 | | |
| 684 | 1 | | |
| 685 | | 61 | 6.2 |
| 686 | 0.5 | | |
| 687 | 0.5 | | |
| 688 | 0.44 | | |
| 689 | | 63 | 41.2 |
| 690 | 3 | | |
| 691 | 1 | | |
| 692 | 1.7 | | |
| 693 | 0.4 | | |
| 694 | 2.9 | | |
| 695 | 0.19 | | |
| 696 | 1.1 | | |
| 697 | 0.4 | | |
| 698 | 0.7 | | |
| 699 | 1.6 | | |
| 700 | 0.5 | | |
| 701 | 0.7 | | |
| 702 | 0.2 | | |
| 703 | 0.16 | | |
| 704 | 0.3 | | |
| 705 | 0.2 | | |
| 706 | 0.18 | | |
| 707 | 0.2 | | |
| 708 | 0.3 | | |
| 709 | 0.4 | | |
| 710 | 0.7 | | |
| 711 | 0.3 | | |
| 712 | 0.5 | | |
| 713 | 0.3 | | |
| 714 | 0.2 | | |
| 715 | 0.4 | | |
| 716 | 0.3 | | |
| 717 | 0.17 | | |
| 718 | 0.4 | | |
| 719 | 0.2 | | |
| 720 | 6 | | |
| 721 | 2 | | |
| 722 | 0.2 | | |
| 723 | 0.2 | | |
| 724 | | | |
| 725 | 0.5 | | |
| 726 | 0.8 | | |
| 727 | 10 | | |
| 728 | 4 | | |
| 729 | 15 | | |
| 730 | | | |
| 731 | 1.9 | | |
| 732 | 1.1 | | |
| 733 | 10 | | |
| 734 | | 46 | 22.2 |
| 735 | 0.9 | | |
| 736 | 0.4 | | |
| 737 | | 72 | 44.7 |
| 738 | 7 | | |
| 739 | 0.18 | | |
| 740 | 6 | | |
| 741 | 0.3 | | |
| 742 | 0.28 | | |
| 743 | 0.2 | | |
| 744 | 0.2 | | |
| 745 | 0.4 | | |
| 746 | 0.3 | | |
| 747 | 0.09 | | |
| 748 | | 73 | 8.1 |
| 749 | 3 | | |
| 750 | 0.2 | | |
| 751 | 0.3 | | |
| 752 | 0.4 | | |
| 753 | 0.7 | | |
| 754 | 0.4 | | |
| 755 | 0.16 | | |
| 756 | | 66 | 45.1 |
| 757 | 1.1 | | |
| 758 | 0.3 | | |
| 759 | 1.6 | | |
| 760 | 0.07 | | |
| 761 | 0.6 | | |
| 762 | 2.1 | | |
| 763 | 1.7 | | |
| 764 | 0.27 | | |
| 765 | 0.19 | | |
| 766 | 0.4 | | |
| 767 | 0.9 | | |
| 768 | 0.4 | | |
| 769 | 13 | | |
| 770 | 2.7 | | |
| 771 | 0.08 | | |
| 772 | 0.08 | | |
| 773 | 1.9 | | |
| 774 | 0.06 | | |
| 775 | | 10 | 52.7 |
| 776 | | 10 | 14.2 |
| 777 | | 44 | 3.3 |
| 778 | | 10 | 13.7 |
| 779 | | 10 | 59.1 |
| 780 | | 53 | 6.8 |
| 781 | | 10 | 58.1 |
| 782 | | 10 | 3.8 |
| 783 | 0.7 | | |
| 784 | 3 | | |
| 785 | 1.1 | | |
| 786 | 4.4 | | |
| 787 | 0.3 | | |
| 788 | 0.5 | | |
| 789 | | 64 | 6.4 |
| 790 | 2 | | |
| 791 | 3 | | |
| 792 | 0.2 | | |
| 793 | 0.6 | | |
| 794 | 0.1 | | |
| 795 | 0.08 | | |
| 796 | 0.6 | | |
| 797 | 0.8 | | |
| 798 | 4 | | |
| 799 | 8 | | |
| 800 | 0.4 | | |
| 801 | 0.18 | | |
| 802 | 0.17 | | |
| 803 | 0.29 | | |
| 804 | 0.3 | | |
| 805 | 0.4 | | |
| 806 | 0.23 | | |
| 807 | 0.9 | | |
| 808 | 0.5 | | |
| 809 | 1.1 | | |
| 810 | 0.29 | | |
| 811 | 0.3 | | |
| 812 | 0.6 | | |
| 813 | 0.4 | | |
| 814 | 0.12 | | |
| 815 | 1.8 | | |
| 816 | 0.27 | | |
| 817 | 0.3 | | |
| 818 | 0.3 | | |
| 819 | 0.22 | | |
| 820 | 0.25 | | |
| 821 | 0.3 | | |
| 822 | 0.2 | | |
| 823 | 0.8 | | |
| 824 | 0.3 | | |
| 825 | 0.13 | | |
| 826 | 0.7 | | |
| 827 | 0.25 | | |
| 828 | 0.3 | | |
| 829 | 0.4 | | |
| 830 | 0.6 | | |
| 831 | 0.26 | | |
| 832 | 0.25 | | |
| 833 | 0.4 | | |
| 834 | 0.4 | | |
| 835 | 1 | | |
| 836 | 0.21 | | |
| 837 | 0.18 | | |
| 838 | 1.2 | | |
| 839 | 0.3 | | |
| 840 | 0.8 | | |
| 841 | 18 | | |
| 842 | 0.19 | | |
| 843 | 0.26 | | |
| 844 | 0.16 | | |
| 845 | 0.5 | | |
| 846 | 0.3 | | |
| 847 | 2.2 | | |
| 848 | 0.7 | | |
| 849 | 0.7 | | |
| 850 | 0.6 | | |
| 851 | 0.4 | | |
| 852 | 0.3 | | |
| 853 | 0.02 | | |
| 854 | 0.02 | | |
| 855 | 0.04 | | |
| 856 | 0.1 | | |
| 857 | 0.15 | | |
| 858 | 1.4 | | |
| 859 | 0.6 | | |
| 860 | 0.8 | | |
| 861 | 0.6 | | |
| 862 | 1.3 | | |
| 863 | 1.8 | | |
| 864 | 0.3 | | |
| 865 | 0.3 | | |
| 866 | 0.8 | | |
| 867 | 0.8 | | |
| 868 | 2.5 | | |
| 869 | 1.2 | | |
| 870 | 0.8 | | |
| 871 | 0.6 | | |
| 872 | 0.6 | | |
| 873 | 1.6 | | |
| 874 | 1.95 | | |
| 875 | 0.6 | | |
| 876 | 0.8 | | |
| 877 | 0.03 | | |
| 878 | 0.27 | | |
| 879 | 0.19 | | |
| 880 | 0.06 | | |
| 881 | 0.28 | | |
| 882 | 0.08 | | |
| 883 | 0.4 | | |
| 884 | 1.6 | | |
| 885 | 0.4 | | |
| 886 | 0.15 | | |
| 887 | 3 | | |
| 888 | 0.9 | | |
| 889 | 4 | | |
| 890 | 0.9 | | |
| 891 | 0.12 | | |
| 893 | 0.7 | | |
| 894 | 0.5 | | |
| 895 | 0.19 | | |
| 896 | 0.3 | | |
| 897 | 0.5 | | |
| 898 | 1 | | |
| 899 | 0.3 | | |
| 900 | 0.08 | | |
| 901 | 0.45 | | |
| 902 | 0.18 | | |
| 903 | 0.07 | | |
| 904 | 0.06 | | |
| 905 | 2.6 | | |
| 906 | 0.9 | | |
| 907 | 0.9 | | |
| 908 | | 74 | 56.3 |
| 909 | 1 | | |
| 910 | | 51 | 12.7 |
| 911 | 4 | | |
| 912 | 2.1 | | |
| 913 | 3 | | |
| 914 | 0.08 | | |
| 915 | 0.3 | | |
| 916 | 0.3 | | |
| 917 | 0.3 | | |
| 918 | 4 | | |
| 919 | 1.4 | | |
| 920 | 0.28 | | |
| 921 | 4 | | |
| 922 | | 62 | 44.5 |
| 923 | 10 | | |
| 924 | 1.8 | | |
| 925 | 8 | | |
| 926 | 0.5 | | |
| 927 | 2.4 | | |
| 928 | 0.18 | | |
| 929 | 4 | | |
| 930 | 0.12 | | |
| 931 | 4 | | |
| 932 | 3 | | |
| 933 | 6 | | |
| 934 | 2.8 | | |
| 935 | 7 | | |
| 936 | 3 | | |
| 937 | 1.7 | | |
| 938 | 0.9 | | |
| 939 | 0.7 | | |
| 940 | 1.1 | | |
| 941 | 0.7 | | |
| 942 | 0.19 | | |
| 943 | 0.4 | | |
| 944 | 1.7 | | |
| 945 | 0.9 | | |
| 946 | 0.5 | | |
| 947 | 0.4 | | |
| 948 | 6 | | |
| 949 | 0.8 | | |
| 950 | 1.8 | | |
| 951 | | 27 | 3.1 |
| 952 | 5 | | |
| 953 | 7 | | |
| 954 | 0.9 | | |
| 955 | 6 | | |
| 956 | 0.11 | | |
| 957 | 0.3 | | |
| 958 | 0.11 | | |
| 959 | 0.4 | | |
| 960 | | 10 | 3.5 |
| 961 | 4 | | |
| 962 | 3 | | |
| 963 | 0.3 | | |
| 964 | | 18 | 67.4 |
| 965 | 0.5 | | |
| 966 | 0.5 | | |
| 967 | 2.5 | | |
| 968 | 1.4 | | |
| 969 | 0.18 | | |
| 970 | 0.1 | | |
| 971 | 0.6 | | |
| 972 | 0.12 | | |
| 973 | | 51 | 0.8 |
| 974 | 4 | | |
| 975 | 1.5 | | |
| 976 | 6 | | |
| 977 | 0.18 | | |
| 978 | 0.25 | | |
| 979 | 2.2 | | |
| 980 | 0.4 | | |
| 981 | 13 | | |
| 982 | 8 | | |
| 983 | 0.8 | | |
| 984 | 0.2 | | |
| 985 | 0.09 | | |
| 986 | 0.8 | | |
| 987 | 0.14 | | |
| 988 | 1.1 | | |
| 989 | 3 | | |
| 990 | 0.13 | | |
| 991 | 0.08 | | |
| 992 | 0.7 | | |
| 993 | 1.5 | | |
| 994 | 2.3 | | |
| 995 | 0.03 | | |
| 996 | 0.3 | | |
| 997 | 0.9 | | |
| 998 | 0.3 | | |
| 999 | 0.8 | | |
| 1000 | 0.2 | | |
| 1001 | 0.11 | | |
| 1002 | 0.1 | | |
| 1003 | 0.14 | | |
| 1004 | 0.06 | | |
| 1005 | 0.14 | | |
| 1006 | 0.18 | | |
| 1007 | 0.9 | | |
| 1008 | 3 | | |
| 1009 | 0.21 | | |
| 1010 | 0.6 | | |
| 1011 | 0.063 | | |
| 1012 | 0.106 | | |
| 1013 | 0.158 | | |
| 1014 | 0.12 | | |
| 1015 | 0.2 | | |
| 1016 | | 50 | 1.56 |
| 1017 | 0.085 | | |
| 1018 | 0.05 | | |
| 1019 | 0.29 | | |
| 1020 | 0.6 | | |
| 1021 | 0.2 | 36 | 1.66 |
| 1022 | 0.17 | | |
| 1023 | 0.21 | | |
| 1024 | 0.2 | | |
| 1025 | 0.13 | | |
| 1026 | | 10 | 57.7 |
| 1027 | | 10 | 51.3 |
| 1028 | 0.06 | | |
| 1029 | 0.14 | | |
| 1030 | 0.5 | | |
| 1031 | 0.02 | | |
| 1032 | 0.5 | | |
| 1033 | 0.5 | | |
| 1034 | 0.5 | | |
| 1035 | 0.3 | | |
| 1036 | 0.8 | | |
| 1037 | 0.08 | | |
| 1038 | 0.1 | | |
| 1039 | 0.4 | | |
| 1040 | 0.14 | | |
| 1041 | 0.16 | | |
| 1042 | 0.13 | | |
| 1043 | 0.09 | | |
| 1044 | 0.5 | | |
| 1045 | 4 | | |
| 1046 | 0.8 | | |
| 1047 | 0.12 | | |
| 1048 | 0.08 | | |
| 1049 | 0.06 | | |
| 1050 | 0.17 | | |
| 1051 | 0.05 | | |
| 1052 | 0.09 | | |
| 1053 | 0.04 | | |
| 1054 | 0.04 | | |
| 1055 | 0.05 | | |
| 1056 | 0.04 | | |
| 1057 | 0.05 | | |
| 1058 | 0.07 | | |
| 1059 | 0.06 | | |
| 1060 | 0.04 | | |
| 1061 | 0.02 | | |
| 1062 | 0.01 | | |
| 1063 | 0.14 | | |
| 1064 | 0.06 | | |
| 1065 | 0.05 | | |
| 1066 | 0.24 | | |
| 1067 | 0.04 | | |
| 1068 | 0.04 | | |
| 1069 | 0.02 | | |
| 1070 | 0.05 | | |
| 1071 | 0.04 | | |
| 1072 | 0.02 | | |
| 1074 | 0.02 | | |
| 1075 | 0.03 | | |
| 1076 | 0.07 | | |
| 1077 | 0.11 | | |
| 1078 | 0.07 | | |
| 1079 | 0.04 | | |
| 1080 | 0.03 | | |
| 1081 | 0.09 | | |
| 1082 | 0.016 | | |
| 1083 | 0.15 | | |
| 1084 | 1.4 | | |
| 1085 | 0.15 | | |
| 1086 | 0.27 | | |
| 1087 | 0.13 | | |
| 1088 | 0.04 | | 25 |
| 1089 | 0.03 | | 12.5 |
| 1090 | 0.06 | | 25 |
| 1091 | 0.03 | | 100 |
| 1092 | 0.03 | | 100 |
| 1093 | 0.03 | | 100 |
| 1094 | 0.11 | | 6.3 |
| 1095 | 0.06 | | 50 |

### EXAMPLE 1097

### Cell-Based Assay for in vitro Measurement of SFRP-1/SARP2 Antagonist Activity

The following cell-based assay can be used to identify inhibitors of SFRP-1.

Material and Methods

Cells

The osteosarcoma cell line, U2OS (ATCC, HTB 96), was passaged twice a week with growth medium (McCoy's 5A medium containing 10% (v/v) fetal calf serum, 2 mM GlutaMAX-1, and 1% (v/v) Penicillin-Streptomycin). The cells were maintained in vented flasks 37° C. inside a 5% CO₂/95% humidified air incubator. One day prior to transfection, the cells were plated with growth medium at 25,000 cells/well into 96-well plates and incubated at 37° C. overnight.

Routine Co-Transfection

The growth medium was removed, and the cells were washed once with OPTIMEM I (Gibco-BRL) medium (100 μL/well) to remove the serum and antibiotics. The wash medium was removed, and the cells were re-fed with OPTI-MEM I medium (100 μL/well). For each well of cells to be transfected, the following DNA's were diluted together in 25 μL OPTI-MEM I medium: 0.1 μg 16× TCF-tk-Luciferase reporter, 0.02 μg Wnt3A, Wnt3A, Wnt 1 or empty vector (Upstate Biotechnology), 0.075 82 g hSFRP-1 or empty vector (pcDNA3.1, Invitrogen), and 0.025 μg CMV-βgal (Clonetech). For each well of cells to be transfected, 1 μl of Lipofectamine 2000 reagent (Invitrogen) was diluted in 25 μl OPTI-MEM I medium and incubated at room temperature for 5 minutes. The diluted DNA's were then combined with the diluted Lipofectamine 2000 (LF2000), and the mixture was incubated at room temperature for 20 minutes. Fifty μL of the DNA-LF 2000 mixture was added to each well, and the plate(s) were incubated at 37° C. in a 5% CO₂/95% humidified air incubator for 4 hours. The cells were washed once with 150 μL/well of experimental medium (phenol red-free RPMI Medium 1640 containing 2% fetal bovine serum, 2 mM GlutaMAX-1, and 1% Penicillin-Streptomycin). Finally, the cells were treated overnight at 37° C. with 200 μL/well of experimental medium containing either vehicle (typically DMSO) or diluted compound in replicates of 8 wells/compound.

Dosing

Initial single dose screening of test compounds was done at 10 μM.

Dose-response experiments were initially performed with the compounds in log increases of concentration from 1-10,000 nM. From these dose-response curves, EC₅₀ values were generated.

Assay

After treatment, the cells were washed twice with 150 μL/well of PBS without calcium or magnesium and were lysed with 50 μl/well of 1× cell culture lysis reagent (Promega Corporation) on a shaker at room temperature for 30 minutes. Thirty μL aliquots of the cell lysates were transferred to 96-well luminometer plates, and luciferase activity was measured in a MicroLumat PLUS luminometer (EG&G Berthold), or a Victor (PerkinElmer Life Sciences) using 100 μL/well of luciferase substrate (Promega Corporation). Following the injection of substrate, luciferase activity was measured for 10 seconds after a 1.6 second delay. Similarly, 10 μL aliquots of the cell lysates were transferred to separate 96-well luminometer plates, and 50 μL of Galacton chemiluminescent substrate (Tropix) was added to each well. The plates were covered and incubated on a rotary shaker at room temperature for one hour. βgal activity was measured in a MicroLumat PLUS luminometer or Victor using 100 μL/well of Light Emission Accelerator (Tropix). Following the injection of the accelerator, βgal activity was measured for 10 seconds after a 1.6 second delay. The luciferase and βgal activity data were transferred from the luminometer to a PC and analyzed using the SAS/Excel program. After the luciferase activity was normalized to βgal, the SAS/Excel program was used to determine the mean and standard deviation of each treatment, to analyze the data for statistical significance, and to determine EC₅₀ values (see the Table below).

Large-Scale Co-Transfection

As an alternative to co-transfection in a 96 well plate, the U2OS cells were transfected in T225 flasks and the transfected cells were frozen. The frozen cells were thawed and plated on a 96 well plate and the assay was carried out as detailed above. The growth medium was removed from the T225 flasks, and the cells were washed once with OPTI-MEM I medium (approx. 25 ml/flask) to remove the serum and antibiotics. The wash medium was removed, and the cells were re-fed with OPTI-MEM I medium (59 ml/flask). For each T225 flask of cells to be transfected, the following DNA's were diluted together in 5.9 ml OPTI-MEM I medium: 70.3 μg 16× TCF-tk-Luciferase reporter, 14.06 μg WNT3, 3A or Wnt1 or empty vector, 52.8 μg hSFRP-1 or empty vector, and 17.58 μg CMV-βgal. Separately, for each flask of cells to be transfected, 354 μL of Lipofectamine 2000 reagent (Invitrogen) was diluted in 5.9 mL OPTI-MEM I medium and incubated at room temperature for 5 minutes. The diluted DNA's were then combined with the diluted Lipofectamine 2000 (LF2000), and the mixture was incubated at room temperature for 20 minutes. 11.8 mL of the DNA-LF 2000 mixture was added to each flask, and the flask(s) were incubated at 37° C. in a 5% CO₂/95% humidified air incubator for 4 hours. The medium was removed, and the cells were washed once with approximately 25 mL/flask of phenol red-free RPMI Medium 1640, then re-fed with 50 mL/flask of experimental medium (phenol red-free RPMI Medium 1640 containing 2% fetal bovine serum, 2 mM GlutaMAX-1, and 1% Penicillin-Streptomycin) and incubated at 37° C. overnight.

Freezing Cells

The transfected cells were washed twice with 25 mL/flask/wash of PBS without calcium or magnesium. Three ml of Trypsin-EDTA (0.05% Trypsin, 0.53 mM EDTA-4Na) was added to each flask, and the flasks were incubated at room temperature for approximately 5 minutes until the cells were rounded and detached from the surface of the flask(s). The cells were resuspended in 10 mL/flask of phenol red-free RPMI 1640 containing 10% fetal bovine serum and were pipetted up and down several times until a single cell suspension was formed. The resuspended cells were pooled and a 10 μL aliquot was removed and diluted at 1:10 in PBS. The diluted cells were counted using a hemacytometer to determine the total number of cells in the pool. The cells were transferred to sterile centrifuge tubes and pelletted at 1500 rpm in a Sorvall RC-3B refrigerated centrifuge at 4° C. for 5 minutes. The supernatant was aspirated and the cells were resuspended in cold, phenol red-free RPMI 1640 medium containing 50% FBS to a cell density of 2.5E+7 cells/ml. An equal volume of cold, 2× freezing medium (phenol red-free RPMI 1640 medium containing 50% FBS and 15% DMSO) was added slowly, dropwise to the resuspended cells with gentle mixing, resulting in a final cell density of 1.25E+7 cells/mL. The resuspended cells were placed on ice and aliquoted into sterile cryogenic vials. The vials were transferred to a Nalgene Cryo 1° C. freezing container (Nalgene catalog #5100-0001) containing 250 mL isopropyl alcohol. The sealed container was placed in a −80° C. freezer overnight to freeze the cells at a cooling rate of −1° C./minute. The frozen cells were then transferred to a −150° C. freezer for long-term storage.

Benchtop Assay for Single Dose Confirmation of HTS Hits

Early in the morning, a vial of frozen transfected cells was thawed, and the cells were resuspended in phenol red-free RPMI 1640 medium to a final cell density of 150,000 cells/ml. The resuspended cells were then plated in white, 96-well polystyrene tissue culture treated CulturPlates™ (Packard cat. #6005180) at a volume of 100 μL of cell suspension/well (i.e. 15,000 cells/well). The plates were incubated at 37° C. inside a 5% CO₂/95% humidified air incubator for 6 hours or until the cells were attached and started to spread. Test compounds were then added to the wells (1 well/compound) and the plates were incubated at 37° C. overnight. After the overnight incubation, luciferase activity was measured using the Luc-Screen Luciferase Assay System (Tropix). Fifty μL of Luc-Screen buffer 1, warmed to room temperature, was added directly to the cells in the 96-well plates. Fifty μL of Luc-Screen buffer 2, warmed to room temperature, was then added, and the plates were incubated in the dark, at room temperature, for 10 minutes. The plates were transferred to a Packard Top Count Microplate Scintillation and Luminescence Counter (Packard), and the light emission was measured for 10 seconds after a 2 minute delay.

The luciferase activity data was transferred to a PC and analyzed using the SAS/Excel program as described above.

Analysis of Results

The luciferase data was analyzed using the SAS/Excel program. For the initial single dose experiment, if the compound treatment resulted in increased reporter activity and was specific to SFRP-1 inhibition, then the results were reported as fold induction over SFRP-1 control (see the Table below).

Compounds

A known inhibitor of GSK-3β, a key enzyme involved in the Wnt signaling pathway, served as an internal control for measurement of the cellular response to Wnt signaling. The inhibition of GSK-3 results in stabilization of β-catenin, leading to up-regulation of LEF/TCF regulated reporter genes.

| | | Fold Induction with | Fold Induction without |
|---|---|---|---|
| Example | Conc (μM) | SFRP-1 | SFRP-1 |
| 1 | 30 | 1.5 | 0.6 |
| 2 | 30 | 0.9 | 0.5 |
| 3 | 30 | 1.1 | 0.4 |
| 4 | 30 | 0.1 | 0.1 |
| 24 | 30 | 0.3 | 0.3 |
| 30 | 30 | 1.7 | 0.7 |
| 30 | 30 | 1 | 0.3 |
| 30 | 15 | 2.4 | 0.9 |
| 31 | 30 | 0.4 | 0.5 |
| 32 | 30 | 0.5 | 0.5 |
| 33 | 30 | 1.2 | 0.4 |
| 34 | 30 | 1.7 | 0.6 |
| 40 | 30 | 1.7 | 0.9 |
| 43 | 30 | 3.6 | 1 |
| 51 | 30 | 2.6 | 0.9 |
| 52 | 30 | 1.4 | 0.7 |
| 53 | 30 | 1 | 0.4 |
| 53 | 30 | 1.2 | 0.6 |
| 54 | 30 | 3.8 | 1.1 |
| 54 | 30 | 3.6 | 1.1 |
| 55 | 30 | 3.1 | 0.9 |
| 62 | 15 | 1.2 | 1.1 |
| 80 | 15 | 1 | 0.7 |
| 81 | 15 | 1 | 0.9 |
| 82 | 15 | 1.2 | 1 |
| 83 | 15 | 1.3 | 1.2 |
| 89 | 30 | 1.8 | 0.5 |
| 92 | 30 | 1 | 0.4 |
| 116 | 30 | 0.9 | 0.4 |
| 117 | 30 | 1 | 0.5 |
| 118 | 30 | 1.3 | 0.5 |
| 119 | 30 | 0.2 | 0.3 |
| 120 | 30 | 1.2 | 0.5 |
| 138 | 30 | 1.1 | 0.5 |
| 138 | 15 | 2.7 | 0.9 |
| 141 | 15 | 3.3 | 1 |
| 142 | 15 | 3.2 | 1.3 |
| 143 | 15 | 3.3 | 0.7 |
| 144 | 15 | 3 | 1.1 |
| 145 | 15 | 2.2 | 1.2 |
| 146 | 15 | 2.1 | 1.3 |
| 147 | 15 | 3.2 | 1 |
| 148 | 15 | 2.8 | 1.1 |
| 149 | 15 | 3.1 | 1.1 |
| 150 | 15 | 1.5 | 1.2 |
| 151 | 15 | 1.4 | 1.1 |
| 152 | 15 | 1.6 | 1.1 |
| 153 | 15 | 1.5 | 1.1 |
| 154 | 15 | 1.4 | 1.3 |
| 155 | 15 | 1.5 | 1.2 |
| 156 | 15 | 1.4 | 1.4 |
| 157 | 15 | 1.5 | 1.2 |
| 158 | 15 | 1.4 | 1.1 |
| 159 | 15 | 1.8 | 0.9 |
| 160 | 15 | 2.4 | 0.9 |
| 161 | 15 | 2.7 | 1 |
| 162 | 15 | 2.7 | 0.8 |
| 162 | 15 | 2.5 | 1 |
| 162 | 15 | 2.7 | 0.8 |
| 162 | 15 | 2.5 | 1 |
| 163 | 15 | 2 | 0.9 |
| 164 | 15 | 1.9 | 1.1 |
| 165 | 15 | 3.3 | 1 |
| 166 | 15 | 2 | 1.1 |
| 167 | 15 | 3 | 1 |
| 168 | 15 | 1.7 | 0.9 |
| 169 | 15 | 2.6 | 0.9 |
| 170 | 15 | 2.3 | 0.9 |
| 171 | 15 | 2.2 | 1.2 |
| 172 | 15 | 1.9 | 1.1 |
| 173 | 15 | 2.5 | 0.8 |
| 173 | 15 | 2 | 1 |
| 173 | 15 | 2.5 | 0.8 |
| 173 | 15 | 2 | 1 |
| 174 | 15 | 2.8 | 1.2 |
| 175 | 15 | 2.5 | 0.9 |
| 176 | 15 | 2.6 | 1.3 |
| 177 | 15 | 3.6 | 1 |
| 177 | 15 | 2.9 | 1 |
| 177 | 15 | 3.6 | 1 |
| 177 | 14 | 2.9 | 1 |
| 178 | 15 | 2.8 | 1.5 |
| 179 | 15 | 2 | 1.1 |
| 180 | 15 | 1.2 | 1 |
| 181 | 30 | 0.6 | 1 |
| 182 | 30 | 1 | 1 |
| 183 | 30 | 1.3 | 1 |
| 184 | 30 | 1.2 | 0.9 |
| 185 | 30 | 1.3 | 1.1 |
| 186 | 30 | 1.3 | 1.3 |
| 187 | 30 | 1.9 | 1 |
| 188 | 30 | 1.4 | 1.4 |
| 189 | 30 | 1.3 | 1 |
| 190 | 30 | 1.2 | 1 |
| 191 | 30 | 1.2 | 1.2 |
| 192 | 30 | 1.1 | 1.1 |
| 193 | 30 | 1.2 | 1.3 |
| 194 | 30 | 1.3 | 1.2 |
| 195 | 30 | 1.2 | 0.9 |
| 196 | 30 | 1.4 | 1.3 |
| 197 | 30 | 1.1 | 1.2 |
| 198 | 30 | 1.1 | 1.3 |
| 199 | 30 | 1.1 | 1 |
| 200 | 30 | 1.4 | 1.8 |
| 201 | 30 | 1.1 | 1.2 |
| 202 | 30 | 1.1 | 1.2 |
| 203 | 15 | 2.5 | 1.3 |
| 204 | 15 | 3.4 | 1.1 |
| 205 | 15 | 2.3 | 0.8 |
| 206 | 15 | 2.2 | 0.5 |
| 207 | 15 | 2.3 | 1.1 |
| 208 | 15 | 2.6 | 0.9 |
| 209 | 15 | 2.2 | 1.3 |
| 210 | 15 | 2.5 | 1 |
| 211 | 15 | 2.3 | 0.7 |
| 212 | 15 | 2 | 0.9 |
| 213 | 15 | 1.8 | 1.3 |
| 214 | 15 | 3.2 | 1.5 |
| 215 | 15 | 2.9 | 1 |
| 216 | 15 | 3.6 | 1.3 |
| 217 | 15 | 2.5 | 1 |
| 218 | 15 | 2.2 | 1.2 |
| 219 | 15 | 3 | 1.1 |
| 219 | 15 | 3.6 | 1.1 |
| 219 | 15 | 3 | 1.1 |
| 219 | 15 | 3.6 | 1.1 |
| 220 | 15 | 3.6 | 1.8 |
| 221 | 15 | 3 | 1.2 |
| 222 | 15 | 2.4 | 0.9 |
| 223 | 15 | 2.6 | 0.8 |
| 224 | 15 | 2.7 | 1 |
| 225 | 15 | 3.1 | 0.9 |
| 226 | 15 | 3 | 0.7 |
| 227 | 15 | 2.7 | 0.8 |
| 228 | 15 | 3.3 | 1.2 |
| 229 | 15 | 2.6 | 0.8 |
| 230 | 15 | 3.2 | 1.2 |
| 231 | 15 | 3.4 | 1 |
| 231 | 15 | 2.8 | 0.8 |
| 231 | 15 | 3.4 | 1 |
| 231 | 15 | 2.8 | 0.8 |
| 232 | 15 | 4.2 | 1.6 |
| 233 | 15 | 1.5 | 1 |
| 234 | 15 | 1.2 | 1 |
| 235 | 15 | 1.2 | 0.9 |
| 236 | 15 | 1.2 | 1 |
| 237 | 15 | 1.1 | 0.9 |
| 237 | 15 | 1.4 | 1.1 |
| 238 | 15 | 1.3 | 0.9 |
| 239 | 15 | 1.2 | 1 |
| 240 | 15 | 1.4 | 0.9 |
| 241 | 15 | 1.8 | 1.1 |
| 242 | 15 | 2.3 | 1.2 |
| 243 | 15 | 2.4 | 1.9 |
| 244 | 15 | 1.3 | 1.1 |
| 245 | 15 | 1.9 | 1.2 |
| 246 | 20 | 2.2 | 0.8 |
| 247 | 20 | 1.7 | 0.9 |
| 248 | 20 | 2.8 | 0.9 |
| 249 | 20 | 1.9 | 0.8 |
| 250 | 20 | 1.4 | 0.9 |
| 251 | 20 | 3 | 0.8 |
| 252 | 20 | 2.6 | 1 |
| 253 | 20 | 1.4 | 0.9 |
| 254 | 20 | 3 | 1 |
| 255 | 20 | 2.6 | 1.1 |
| 256 | 20 | 1.2 | 1 |
| 257 | 20 | 2.4 | 1 |
| 258 | 20 | 1.2 | 1 |
| 259 | 20 | 1 | 1.1 |
| 260 | 20 | 1.2 | 1 |
| 261 | 15 | 1.4 | 0.9 |
| 262 | 15 | 1.6 | 1.1 |
| 263 | 15 | 0.8 | 1.1 |
| 264 | 15 | 1.1 | 0.9 |
| 264 | 15 | 1.7 | 1.1 |
| 265 | 15 | 1.1 | 1.1 |
| 265 | 15 | 1.2 | 1 |
| 266 | 15 | 2.7 | 1.2 |
| 267 | 15 | 2 | 1.2 |
| 268 | 15 | 3.6 | 1.1 |
| 269 | 15 | 1.4 | 1.2 |
| 270 | 15 | 1.5 | 1.2 |
| 271 | 15 | 1.7 | 1.2 |
| 272 | 15 | 0.9 | 1 |
| 273 | 15 | 0.9 | 1 |
| 274 | 15 | 2 | 1 |
| 275 | 15 | 0.8 | 0.8 |
| 276 | 15 | 0.8 | 1 |
| 277 | 15 | 1.8 | 1 |
| 278 | 15 | 1 | 0.9 |
| 279 | 15 | 1.9 | 1 |
| 280 | 15 | 2.7 | 1.5 |
| 281 | 15 | 2.8 | 1.3 |
| 282 | 15 | 2.4 | 1.6 |
| 283 | 15 | 1.8 | 1.3 |
| 284 | 15 | 1.1 | 1.2 |
| 285 | 15 | 2 | 1.1 |
| 286 | 15 | 2.0 | 1.3 |
| 287 | 15 | 2.1 | 1.2 |
| 288 | 15 | 3.6 | 1.7 |
| 289 | 15 | 2.3 | 1.1 |
| 290 | 15 | 1.9 | 0.8 |
| 291 | 15 | 1.7 | 0.9 |
| 292 | 15 | 2.3 | 0.8 |
| 293 | 15 | 2.5 | 0.9 |
| 294 | 15 | 2.3 | 1.5 |
| 295 | 15 | 2.7 | 1.5 |
| 296 | 15 | 3.3 | 1.4 |
| 297 | 15 | 4.3 | 1.4 |
| 298 | 15 | 3.2 | 1.3 |
| 299 | 15 | 3.1 | 1.1 |
| 300 | 15 | 3.4 | 1.2 |
| 301 | 15 | 3.3 | 1.2 |
| 302 | 15 | 1.2 | 1.2 |
| 303 | 15 | 2.7 | 0.8 |
| 304 | 15 | 1.7 | 1 |
| 305 | 15 | 1.8 | 1 |
| 306 | 60 | 1.3 | 0.9 |
| 307 | 15 | 2.5 | 0.9 |
| 308 | 15 | 2.3 | 0.9 |
| 309 | 15 | 1.9 | 0.9 |
| 310 | 15 | 2.9 | 1.1 |
| 311 | 15 | 2.4 | 1 |
| 312 | 15 | 1.6 | 0.9 |
| 313 | 15 | 1.9 | 1 |
| 314 | 15 | 3.3 | 1.3 |
| 315 | 15 | 1.8 | 1.3 |
| 316 | 15 | 2.5 | 1.1 |
| 317 | 15 | 1.2 | 1.1 |
| 318 | 15 | 3.9 | 1.1 |
| 319 | 15 | 3.5 | 1.1 |
| 320 | 15 | 2.8 | 1.2 |
| 321 | 15 | 3.3 | 1.2 |
| 322 | 15 | 2.5 | 1.1 |
| 323 | 15 | 2.6 | 0.9 |
| 324 | 15 | 3.2 | 0.9 |
| 325 | 15 | 3 | 1.1 |
| 326 | 15 | 2.4 | 1 |
| 327 | 15 | 2.7 | 1 |
| 328 | 15 | 2.6 | 1.1 |
| 329 | 15 | 2.9 | 0.9 |
| 330 | 15 | 3.2 | 1.1 |
| 331 | 15 | 2.3 | 1.1 |
| 332 | 15 | 3 | 1 |
| 333 | 15 | 1.7 | 1 |
| 334 | 15 | 1.5 | 1.2 |
| 335 | 15 | 2.2 | 1 |
| 336 | 15 | 2.8 | 1 |
| 337 | 15 | 2.6 | 1.1 |
| 338 | 15 | 1.2 | 1 |
| 339 | 15 | 2.6 | 1.1 |
| 340 | 15 | 2.5 | 1 |
| 341 | 15 | 1.5 | 1.1 |
| 342 | 15 | 3.3 | 1.1 |
| 343 | 15 | 2.8 | 1 |
| 344 | 15 | 2.2 | 1 |
| 345 | 15 | 1.6 | 1 |
| 346 | 15 | 2.4 | 1.3 |
| 347 | 15 | 2.9 | 1.1 |
| 348 | 15 | 2.5 | 1.2 |
| 349 | 15 | 2 | 1.2 |
| 350 | 15 | 1.8 | 1.3 |
| 351 | 15 | 1.5 | 1.3 |
| 352 | 15 | 1.7 | 1 |
| 353 | 15 | 3.3 | 1.1 |
| 354 | 15 | 2.6 | 1 |
| 355 | 15 | 3.4 | 0.9 |
| 356 | 15 | 2.5 | 1 |
| 357 | 15 | 4.2 | 1.1 |
| 358 | 15 | 2.9 | 1 |
| 359 | 15 | 2.7 | 0.9 |
| 360 | 15 | 1.9 | 0.9 |
| 361 | 15 | 2.3 | 0.9 |
| 362 | 15 | 2.4 | 1.1 |
| 363 | 15 | 2.1 | 1.1 |
| 364 | 15 | 1.2 | 1 |
| 365 | 15 | 1.3 | 1.1 |
| 366 | 15 | 2.4 | 1 |
| 367 | 15 | 2.2 | 0.9 |
| 368 | 15 | 2.5 | 1 |
| 369 | 15 | 1.9 | 0.7 |
| 370 | 15 | 1.8 | 0.8 |
| 371 | 15 | 1.4 | 0.8 |
| 372 | 15 | 2.2 | 0.9 |
| 373 | 15 | 2.8 | 1 |
| 374 | 15 | 1.5 | 0.9 |
| 375 | 15 | 1.9 | 0.9 |
| 376 | 15 | 1.5 | 1.1 |
| 377 | 15 | 1.9 | 1.1 |
| 378 | 15 | 1.7 | 1.1 |
| 379 | 15 | 1.8 | 0.9 |
| 380 | 15 | 0.5 | 0.6 |
| 381 | 15 | 2.1 | 0.8 |
| 382 | 15 | 2.4 | 0.9 |
| 383 | 15 | 2.1 | 0.9 |
| 384 | 15 | 2.6 | 0.8 |
| 385 | 15 | 2.7 | 1 |
| 386 | 15 | 1.8 | 1 |
| 387 | 15 | 1.7 | 1.4 |
| 388 | 15 | 2.2 | 1.1 |
| 389 | 15 | 1.5 | 1.1 |
| 390 | 15 | 2.2 | 1.1 |
| 391 | 15 | 1.8 | 1.1 |
| 392 | 15 | 2.8 | 1 |
| 393 | 7.5 | 1.2 | 1 |
| 394 | 15 | 1.5 | 1 |
| 395 | 15 | 1.2 | 1 |
| 396 | 15 | 1.4 | 1 |
| 397 | 15 | 1.2 | 1.1 |
| 398 | 15 | 2.6 | 1.2 |
| 399 | 15 | 2.5 | 1.1 |
| 400 | 15 | 1.8 | 1.2 |
| 401 | 15 | 1.6 | 1.1 |
| 402 | 15 | 2.5 | 1.1 |
| 403 | 15 | 2.5 | 1.1 |
| 404 | 15 | 1.2 | 1.1 |
| 405 | 15 | 2.1 | 1.1 |
| 406 | 60 | 1.2 | 0.9 |
| 407 | 15 | 1.2 | 0.9 |
| 408 | 15 | 1.2 | 1 |
| 409 | 15 | 2.2 | 1.3 |
| 410 | 15 | 2.7 | 1.3 |
| 411 | 15 | 1.6 | 1 |
| 412 | 15 | 1.9 | 1.5 |
| 413 | 15 | 2.2 | 1.4 |
| 414 | 15 | 2 | 1.3 |
| 415 | 15 | 1.5 | 1.2 |
| 416 | 15 | 1.9 | 0.9 |
| 417 | 15 | 1.8 | 1 |
| 418 | 15 | 2.3 | 1.2 |
| 419 | 15 | 2.5 | 1.3 |
| 420 | 15 | 2.4 | 1.3 |
| 421 | 15 | 2.2 | 1.2 |
| 422 | 15 | 1.9 | 1.2 |
| 423 | 15 | 2.4 | 1.1 |
| 424 | 15 | 2.1 | 1.2 |
| 425 | 15 | 2.5 | 1.2 |
| 426 | 15 | 1.3 | 0.8 |
| 427 | 15 | 2 | 0.9 |
| 428 | 15 | 1.3 | 0.8 |
| 429 | 15 | 0.9 | 0.8 |
| 430 | 15 | 1.7 | 0.8 |
| 431 | 15 | 1.7 | 0.8 |
| 432 | 15 | 1.9 | 0.9 |
| 433 | 15 | 2.5 | 1.1 |
| 434 | 15 | 2.1 | 1.1 |
| 435 | 15 | 1.7 | 1 |
| 436 | 15 | 2.2 | 1 |
| 437 | 15 | 2.2 | 1 |
| 438 | 15 | 1.6 | 1 |
| 439 | 15 | 2 | 0.9 |
| 440 | 15 | 1.8 | 1.1 |
| 441 | 15 | 1.8 | 1.1 |
| 442 | 15 | 3.9 | 1 |
| 443 | 15 | 2.5 | 1.1 |
| 444 | 15 | 1.4 | 1.1 |
| 445 | 15 | 2.9 | 1.2 |
| 446 | 15 | 2.3 | 1.1 |
| 447 | 15 | 3 | 1.1 |
| 448 | 15 | 2 | 1.2 |
| 449 | 15 | 2.8 | 1.1 |
| 450 | 15 | 4 | 1 |
| 451 | 15 | 3.7 | 1.1 |
| 452 | 15 | 3.8 | 1 |
| 453 | 15 | 4 | 1.1 |
| 454 | 15 | 1.7 | 1.1 |
| 455 | 15 | 3.2 | 1 |
| 456 | 15 | 3.6 | 1 |
| 457 | 15 | 2.9 | 1 |
| 458 | 15 | 2.9 | 0.9 |
| 459 | 15 | 1.6 | 0.9 |
| 460 | 15 | 1.1 | 1 |
| 461 | 15 | 1.7 | 1 |
| 462 | 15 | 2.9 | 1 |
| 463 | 15 | 2.9 | 1 |
| 464 | 15 | 2.1 | 1 |
| 465 | 15 | 1.9 | 1.1 |
| 466 | 15 | 1.7 | 1.1 |
| 467 | 15 | 2.3 | 1 |
| 468 | 15 | 2 | 1.2 |
| 469 | 15 | 3.6 | 1.1 |
| 470 | 15 | 1.6 | 1.1 |
| 471 | 15 | 2.1 | 1.1 |
| 472 | 15 | 0.9 | 0.9 |
| 473 | 15 | 0.9 | 0.9 |
| 474 | 15 | 1.4 | 1 |
| 475 | 15 | 1.9 | 1.1 |
| 476 | 15 | 2 | 1 |
| 477 | 15 | 2.1 | 1 |
| 478 | 15 | 2.3 | 1 |
| 479 | 15 | 2.1 | 1.1 |
| 480 | 15 | 1.7 | 1 |
| 481 | 15 | 1.8 | 1 |
| 482 | 15 | 3.2 | 1.1 |
| 483 | 15 | 2.9 | 1 |
| 484 | 15 | 2.4 | 1.1 |
| 485 | 15 | 2.5 | 1 |
| 486 | 15 | 2.1 | 1.1 |
| 487 | 15 | 2.1 | 1.1 |
| 488 | 15 | 2 | 0.9 |
| 489 | 15 | 2.4 | 1.1 |
| 490 | 15 | 1.7 | 1 |
| 491 | 15 | 1.1 | 1 |
| 492 | 15 | 1.6 | 1.2 |
| 493 | 15 | 1 | 1.1 |
| 494 | 15 | 1 | 1.1 |
| 495 | 15 | 1.1 | 1.3 |
| 496 | 15 | 1.9 | 1 |
| 497 | 15 | 2.4 | 1 |
| 498 | 15 | 1.8 | 1 |
| 499 | 15 | 2.4 | 1 |
| 500 | 15 | 1.6 | 0.8 |
| 501 | 15 | 2.5 | 0.9 |
| 502 | 15 | 2.9 | 1 |
| 503 | 15 | 2.7 | 0.9 |
| 504 | 15 | 2.2 | 1 |
| 505 | 15 | 2.3 | 1 |
| 506 | 15 | 2.7 | 1 |
| 507 | 15 | 2.7 | 0.9 |
| 508 | 15 | 2.3 | 0.9 |
| 509 | 15 | 1.9 | 1 |
| 510 | 15 | 1.9 | 1.1 |
| 511 | 15 | 2.8 | 0.9 |
| 512 | 15 | 3.1 | 1.1 |
| 513 | 15 | 2.2 | 1.2 |
| 514 | 15 | 2.6 | 1 |
| 515 | 15 | 1.8 | 1 |
| 516 | 15 | 3 | 1 |
| 517 | 15 | 2.2 | 1 |
| 518 | 15 | 3 | 1.2 |
| 519 | 15 | 3 | 0.9 |
| 520 | 15 | 3.3 | 1.1 |
| 521 | 15 | 1.7 | 1 |
| 522 | 15 | 1.4 | 0.9 |
| 523 | 15 | 2.1 | 1 |
| 524 | 15 | 1.4 | 1 |
| 525 | 15 | 2.1 | 1.1 |
| 526 | 15 | 2.1 | 1.1 |
| 527 | 15 | 2.3 | 1.2 |
| 528 | 15 | 2.5 | 1.1 |
| 529 | 15 | 2.8 | 1 |
| 530 | 15 | 3 | 1 |
| 531 | 15 | 2.5 | 1.1 |
| 532 | 15 | 2.8 | 1.1 |
| 533 | 15 | 3.7 | 1.1 |
| 534 | 15 | 4 | 1.1 |
| 535 | 15 | 3.8 | 1.2 |
| 536 | 15 | 3 | 1.2 |
| 537 | 15 | 2.9 | 1.1 |
| 538 | 15 | 2.4 | 1.1 |
| 539 | 15 | 2.9 | 1.2 |
| 540 | 15 | 3.6 | 1.2 |
| 541 | 15 | 3.5 | 1.2 |
| 542 | 15 | 2.7 | 1.1 |
| 543 | 15 | 2.7 | 1.1 |
| 544 | 15 | 2.7 | 1.2 |
| 545 | 15 | 2.5 | 1.1 |
| 546 | 15 | 2.2 | 1.2 |
| 547 | 15 | 3.7 | 1.3 |
| 548 | 15 | 2.5 | 1.2 |
| 549 | 15 | 3.6 | 1.5 |
| 550 | 15 | 2.9 | 1.1 |
| 551 | 15 | 3.1 | 1.1 |
| 552 | 15 | 2.4 | 1.2 |
| 553 | 15 | 2.5 | 1.1 |
| 554 | 15 | 2.6 | 1.1 |
| 555 | 15 | 3.1 | 1.2 |
| 556 | 15 | 4 | 1.2 |
| 557 | 15 | 1.4 | 1.2 |
| 558 | 15 | 1.8 | 1.1 |
| 559 | 15 | 2.3 | 1 |
| 560 | 15 | 1.6 | 0.9 |
| 561 | 15 | 2.3 | 0.9 |
| 562 | 15 | 3.3 | 1.2 |
| 563 | 15 | 2.8 | 1.2 |
| 564 | 15 | 3.3 | 1.3 |
| 565 | 15 | 4 | 1.2 |
| 566 | 15 | 1.5 | 1.3 |
| 567 | 15 | 2.2 | 1.3 |
| 568 | 15 | 3.8 | 1.1 |
| 569 | 15 | 3.8 | 1.1 |
| 570 | 15 | 2.4 | 1.2 |
| 571 | 15 | 3.1 | 1.2 |
| 572 | 15 | 1.7 | 1 |
| 573 | 15 | 2.1 | 1.1 |
| 574 | 15 | 2.1 | 1.2 |
| 575 | 15 | 2.6 | 1.1 |
| 576 | 15 | 3 | 1.2 |
| 577 | 15 | 1.9 | 1.2 |
| 578 | 15 | 2.8 | 1.2 |
| 579 | 15 | 3.4 | 1 |
| 580 | 15 | 2.8 | 1.3 |
| 581 | 15 | 2.2 | 1.3 |
| 582 | 15 | 2.6 | 1 |
| 583 | 15 | 3.9 | 1.1 |
| 584 | 15 | 3.4 | 1.1 |
| 585 | 15 | 2.7 | 1.1 |
| 586 | 15 | 1.9 | 1.1 |
| 587 | 15 | 1.2 | 1.2 |
| 588 | 15 | 3.6 | 1.7 |
| 589 | 15 | 2.6 | 1.3 |
| 590 | 15 | 2.9 | 1 |
| 591 | 15 | 3.1 | 1.3 |
| 592 | 15 | 3.1 | 1.1 |
| 593 | 15 | 2 | 1.1 |
| 594 | 15 | 2.3 | 1 |
| 595 | 15 | 3.4 | 1.4 |
| 596 | 15 | 4.2 | 1.3 |
| 597 | 15 | 3.7 | 1.3 |
| 598 | 15 | 3.6 | 1.4 |
| 599 | 15 | 4.2 | 1.4 |
| 600 | 15 | 3 | 1.1 |
| 601 | 15 | 2 | 0.9 |
| 602 | 15 | 1.3 | 1.1 |
| 603 | 15 | 2.4 | 0.9 |
| 604 | 15 | 2.2 | 1.1 |
| 605 | 15 | 3.2 | 1.1 |
| 606 | 15 | 1.9 | 1 |
| 607 | 15 | 2.3 | 1.1 |
| 608 | 15 | 3.8 | 1.1 |
| 609 | 15 | 1.4 | 1.1 |
| 610 | 15 | 2.5 | 1.1 |
| 611 | 15 | 2.6 | 1.1 |
| 612 | 15 | 2 | 1.1 |
| 613 | 15 | 1.3 | 1 |
| 614 | 15 | 1.8 | 1 |
| 615 | 15 | 1.7 | 1 |
| 616 | 15 | 2.7 | 1 |
| 617 | 15 | 2 | 1.2 |
| 618 | 15 | 2.8 | 1.1 |
| 619 | 15 | 1.3 | 1.1 |
| 620 | 15 | 2.7 | 1 |
| 621 | 15 | 1.2 | 1 |
| 622 | 15 | 2.7 | 1.1 |
| 623 | 15 | 2.2 | 1.1 |
| 624 | 15 | 2.7 | 1.2 |
| 625 | 15 | 2.2 | 1.1 |
| 626 | 15 | 2.9 | 1.1 |
| 627 | 15 | 3.4 | 1.1 |
| 628 | 15 | 3.2 | 1.1 |
| 629 | 15 | 2.1 | 1 |
| 630 | 15 | 2.2 | 1 |
| 631 | 15 | 2.4 | 1 |
| 632 | 15 | 3 | 1 |
| 633 | 15 | 1.5 | 1 |
| 634 | 15 | 3 | 1 |
| 635 | 15 | 2 | 1.7 |
| 636 | 15 | 1.7 | 1.1 |
| 637 | 15 | 0.9 | 1 |
| 638 | 15 | 1.2 | 1.1 |
| 639 | 15 | 0.9 | 1 |
| 640 | 15 | 3.5 | 1.2 |
| 641 | 15 | 3 | 1 |
| 642 | 15 | 2.2 | 1.1 |
| 643 | 15 | 3.2 | 1 |
| 644 | 15 | 1.3 | 1.1 |
| 645 | 15 | 1 | 1.1 |
| 646 | 15 | 1 | 1.1 |
| 647 | 15 | 3.9 | 1.1 |
| 648 | 15 | 2.9 | 1.1 |
| 649 | 15 | 3 | 1.1 |
| 650 | 15 | 3.4 | 1.1 |
| 651 | 15 | 2.9 | 0.9 |
| 652 | 15 | 3.8 | 1 |
| 653 | 15 | 1 | 1 |
| 654 | 15 | 1 | 1 |

| | Fold Induction | EC50 |
|---|---|---|
| Example | with SFRP-1 | (μM) |
| 655 | 1.1 | |
| 656 | 2.2 | 11.67 |
| 657 | 2.4 | 9.03 |
| 658 | 2.6 | 6.35 |
| 659 | | |
| 660 | 4.9 | 3.72 |
| 662 | 3.3 | 17.63 |
| 663 | 1 | |
| 664 | 3.9 | 2.99 |
| 665 | 1.3 | |
| 666 | 1.5 | 27.15 |
| 667 | 0.9 | |
| 668 | 1.4 | |
| 669 | 0.9 | |
| 670 | 3.2 | 2.83 |
| 671 | 3.6 | 1.583 |
| 672 | 4.2 | 3.12 |
| 673 | 2 | 15.88 |
| 674 | 2 | 24.1 |
| 675 | 1.7 | 41.5 |
| 676 | 2.6 | 8.9 |
| 677 | 3.8 | 0.489 |
| 678 | 1.6 | |
| 679 | 3.4 | 0.449 |
| 680 | 3.2 | 0.968 |
| 681 | 3.4 | 4.3 |
| 682 | 1.2 | 0.765 |
| 683 | 3.7 | 3.1 |
| 684 | 1.7 | 29.3 |
| 685 | 1.5 | |
| 686 | 3.5 | 1.747 |
| 687 | 3.2 | 0.261 |
| 688 | 4.7 | 0.7 |
| 689 | 1.2 | |
| 690 | 2.3 | 8.4 |
| 691 | 2.8 | 8.6 |
| 692 | 3 | 8.5 |
| 693 | 3.5 | 1.076 |
| 694 | 2.6 | 8.7 |
| 695 | 2.1 | 7.1 |
| 696 | 2.2 | 14.7 |
| 697 | 3.7 | 0.452 |
| 698 | 3 | 0.609 |
| 699 | 2.9 | 9.8 |
| 700 | 1.7 | 0.397 |
| 701 | 3 | 0.698 |
| 702 | 3.7 | 1.939 |
| 703 | 4.8 | 1.2 |
| 704 | 3.8 | 0.868 |
| 705 | 3.5 | 0.787 |
| 706 | 3.7 | 0.503 |
| 707 | 4.2 | 0.187 |
| 708 | 3.7 | 1.173 |
| 709 | 4.5 | 2.35 |
| 710 | 3.5 | 10 |
| 711 | 3.3 | 9.4 |
| 712 | 2.9 | 10 |
| 713 | 3.9 | 1.829 |
| 714 | 4.2 | 0.597 |
| 715 | 4.9 | 1.293 |
| 716 | 4.2 | 1.758 |
| 717 | 5.3 | 1.29 |
| 718 | 6.7 | 0.214 |
| 719 | 2.1 | 0.441 |
| 720 | 2 | |
| 721 | 2.7 | |
| 722 | 3.4 | 0.629 |
| 723 | 4.5 | 0.79 |
| 724 | 1.7 | |
| 725 | 3.1 | 1.256 |
| 726 | 3 | |
| 727 | 3.2 | |
| 728 | 3.1 | |
| 729 | 1.7 | |
| 730 | 2.2 | |
| 731 | 3.3 | |
| 732 | 4.2 | |
| 733 | 2.5 | |
| 734 | 1.6 | |
| 735 | 4.1 | |
| 736 | 5 | 1.756 |
| 737 | 2.4 | |
| 738 | 2.9 | |
| 739 | 5.1 | 0.72 |
| 740 | 3.3 | |
| 741 | 4.8 | 1.003 |
| 742 | 2.4 | |
| 743 | 5.9 | 1.321 |
| 744 | 3.1 | 0.852 |
| 745 | 4.5 | 0.631 |
| 746 | 5 | 0.453 |
| 747 | 5.7 | 0.779 |
| 748 | 2.8 | |
| 749 | 1.7 | |
| 750 | 4.2 | 0.937 |
| 751 | 3.1 | 4.849 |
| 752 | 3.8 | 1.094 |
| 753 | 3.8 | |
| 754 | 2.5 | 1.98 |
| 755 | 3.8 | 1.43 |
| 756 | 1.3 | |
| 757 | 3.1 | |
| 758 | 3.4 | 0.582 |
| 759 | 3.5 | |
| 760 | 3.5 | 0.253 |
| 761 | 3.2 | 1.574 |
| 762 | 2.2 | |
| 763 | 2.7 | |
| 764 | 0.6 | 0.463 |
| 765 | 3.7 | 0.754 |
| 766 | 2.7 | 1.224 |
| 767 | 3.6 | |
| 768 | 3.4 | 1.129 |
| 769 | | |
| 770 | 1.7 | |
| 771 | 3.5 | 0.733 |
| 772 | 1.2 | |
| 773 | 1.2 | 1.3 |
| 774 | 1.3 | 0.973 |
| 775 | 1.3 | |
| 776 | 0.9 | |
| 777 | 1.1 | |
| 778 | 1.2 | |
| 779 | 1 | |
| 780 | 1.7 | |
| 781 | 1.2 | |
| 782 | 1.2 | |
| 783 | 3.2 | |
| 784 | 3 | |
| 785 | 2.9 | |
| 786 | 1.7 | |
| 787 | 3.7 | 0.896 |
| 788 | 4.7 | 0.85 |
| 789 | 1.5 | |
| 790 | 3.1 | |
| 791 | 3.8 | |
| 792 | 1.4 | 2.29 |
| 793 | 2.5 | 1.536 |
| 794 | 3.3 | 0.534 |
| 795 | 3.8 | 0.14 |
| 796 | 4.3 | 1.35 |
| 797 | 3.5 | |
| 798 | 1.8 | |
| 799 | 2.2 | |
| 800 | 3.6 | 0.738 |
| 801 | 3.4 | 1.08 |
| 802 | 2.6 | 0.301 |
| 803 | 3.2 | 10 |
| 804 | 3 | 10 |
| 805 | 3 | 0.512 |
| 806 | 2.8 | 0.238 |
| 807 | 3 | |
| 808 | 3.7 | 0.945 |
| 809 | 2.8 | |
| 810 | 3.1 | 0.818 |
| 811 | 2.6 | 10 |
| 812 | 3.9 | 10 |
| 813 | 3.9 | 0.674 |
| 814 | 4.5 | 0.307 |
| 815 | 2.2 | |
| 816 | 3.8 | 1.505 |
| 817 | 3.3 | 1.144 |
| 818 | 2.6 | 1.322 |
| 819 | 3.3 | 1.571 |
| 820 | 4.1 | 0.797 |
| 821 | 5.1 | 0.626 |
| 822 | 3.4 | 1.837 |
| 823 | 2.6 | 3 |
| 824 | 3.1 | 0.501 |
| 825 | 4.4 | 0.207 |
| 826 | 3.5 | 1.916 |
| 827 | 3.5 | 1.71 |
| 828 | 2.8 | 2.357 |
| 829 | 2.7 | 1.32 |
| 830 | 3.3 | 1.865 |
| 831 | 3.7 | 1.464 |
| 832 | 3.4 | 2.188 |
| 833 | 3.4 | 1.261 |
| 834 | 3 | 1.495 |
| 835 | 3.3 | |
| 836 | 2.8 | 1.99 |
| 837 | 3.4 | 0.644 |
| 838 | 2.5 | |
| 839 | 3.5 | 2.385 |
| 840 | 2.9 | |
| 841 | 1.6 | |
| 842 | 4.2 | 0.67 |
| 843 | 4.7 | 2.013 |
| 844 | 4.4 | 0.5 |
| 845 | 3.8 | 0.807 |
| 846 | 3.2 | 2.673 |
| 847 | 2.4 | |
| 848 | 3.6 | 1.642 |
| 849 | 3.9 | 2.099 |
| 850 | 3.5 | 1.525 |
| 851 | 3.6 | 1.506 |
| 852 | 3.3 | 1.651 |
| 853 | 4.4 | 0.142 |
| 854 | 4.1 | 0.113 |
| 855 | 3.4 | 0.137 |
| 856 | 4.2 | 0.36 |
| 857 | 4.4 | 1.19 |
| 858 | 3.8 | |
| 859 | 3.6 | 1.535 |
| 860 | 4.1 | |
| 861 | 4 | 1.778 |
| 862 | 4 | |
| 863 | 2.4 | |
| 864 | 3.2 | 1.8 |
| 865 | 2.9 | 2.142 |
| 866 | 4.6 | |
| 867 | 2.8 | |
| 868 | 2.4 | 2.13 |
| 869 | 3.5 | 1.76 |
| 870 | 2.5 | |
| 871 | 2 | 3.812 |
| 872 | 3.1 | 1.948 |
| 873 | 2.1 | |
| 874 | 3.1 | 2 |
| 875 | 1.6 | 6.65 |
| 876 | 3 | 1.36 |
| 877 | 3.9 | 0.257 |
| 878 | 3.5 | 0.399 |
| 879 | 3.2 | 0.567 |
| 880 | 4 | 0.314 |
| 881 | 3.4 | 0.85 |
| 882 | 3.4 | 0.627 |
| 883 | 2.7 | 2.57 |
| 884 | 2.7 | 3.4 |
| 885 | 2.7 | 2.09 |
| 886 | 2.7 | 1.33 |
| 887 | 2.3 | 1.77 |
| 888 | 2.1 | 1.77 |
| 889 | 1.3 | |
| 890 | 3.6 | 1.16 |
| 891 | 3.2 | 0.346 |
| 893 | 1.8 | |
| 894 | 3.5 | 0.574 |
| 895 | 2.9 | 0.871 |
| 896 | 2.9 | 0.675 |
| 897 | 2.7 | 1.36 |
| 898 | 3.1 | 1.93 |
| 899 | 4.2 | 0.894 |
| 900 | 4.1 | |
| 901 | 3.4 | 0.224 |
| 902 | 3 | 0.636 |
| 903 | 3.5 | 0.795 |
| 904 | 3.5 | 0.7 |
| 905 | 1.5 | 3.66 |
| 906 | 2.9 | 1.34 |
| 907 | 2.2 | 10 |
| 908 | 1.1 | |
| 909 | 2.3 | 2.58 |
| 910 | 1.2 | |
| 911 | 1.9 | 2.42 |
| 912 | 2.4 | 1.01 |
| 913 | 3.1 | 7.01 |
| 914 | 4.5 | 0.541 |
| 915 | 3.6 | 0.415 |
| 916 | 3.1 | 0.276 |
| 917 | 3.6 | 0.186 |
| 918 | 1.6 | 2.98 |
| 919 | 1.6 | 5.26 |
| 920 | 2.2 | 1.81 |
| 921 | 2.5 | 2.36 |
| 922 | 1.1 | |
| 923 | 2.4 | 1.87 |
| 924 | 2.5 | 1.92 |
| 925 | 3 | 2.15 |
| 926 | 3.6 | 1.36 |
| 927 | 2.1 | 4.51 |
| 928 | 3.9 | 0.38 |
| 929 | 2.9 | 3.32 |
| 930 | 3.7 | 0.15 |
| 931 | 2.9 | 1.3 |
| 932 | 2.8 | 1.86 |
| 933 | 2.5 | 1.74 |
| 934 | 2.3 | 2.73 |
| 935 | 2.5 | 2.53 |
| 936 | 2.7 | 1.82 |
| 937 | 2.8 | 2.3 |
| 938 | 4.4 | 0.85 |
| 939 | 2.2 | 10 |
| 940 | 4.1 | 2.28 |
| 941 | 3.2 | 1.34 |
| 942 | 3.8 | 0.209 |
| 943 | 3.4 | 0.569 |
| 944 | 2.9 | 1.44 |
| 945 | 2.9 | 1.12 |
| 946 | 3 | 1.46 |
| 947 | 3.2 | 0.695 |
| 948 | 2.4 | 2.74 |
| 949 | 3.2 | 1 |
| 950 | 2.6 | 1.84 |
| 951 | 1.8 | |
| 952 | 1.4 | 10 |
| 953 | 1.5 | |
| 954 | 2.2 | 5.57 |
| 955 | 2.4 | 5.16 |
| 956 | 4.3 | 0.31 |
| 957 | 3.9 | 1.39 |
| 958 | 4.5 | 0.135 |
| 959 | 3.7 | 0.39 |
| 960 | 1.4 | |
| 961 | 2.8 | 2.55 |
| 962 | 2.7 | 2.86 |
| 963 | 3.5 | 1.16 |
| 964 | 1.2 | |
| 965 | 3.6 | 0.401 |
| 966 | 1.5 | 0.229 |
| 967 | 2.1 | 10 |
| 968 | 2.4 | |
| 969 | 3.1 | 0.588 |
| 970 | 2.8 | 0.214 |
| 971 | 2.7 | 0.774 |
| 972 | | 0.226 |
| 973 | | |
| 974 | | |
| 975 | | 5.7 |
| 976 | | |
| 977 | | 1.1 |
| 978 | | 0.392 |
| 979 | | 2.94 |
| 980 | | 0.853 |
| 981 | | |
| 982 | | |
| 983 | | 1.14 |
| 984 | | 0.657 |
| 985 | | 0.174 |
| 986 | | |
| 987 | | 0.231 |
| 988 | | 1.89 |
| 989 | | |
| 990 | | 0.577 |
| 991 | | 2.64 |
| 992 | | 3.16 |
| 993 | | 2.29 |
| 994 | | |
| 995 | | 1.24 |
| 996 | | 3.01 |
| 997 | | 3.08 |
| 998 | | 2.2 |
| 999 | | 0.789 |
| 1000 | | 0.618 |
| 1001 | | 0.194 |
| 1002 | | 0.161 |
| 1003 | | 0.399 |
| 1004 | | 0.473 |
| 1005 | | 0.251 |
| 1006 | | 8.16 |
| 1007 | | 1.52 |
| 1008 | | 0.936 |
| 1009 | | 0.945 |
| 1010 | | 2 |
| 1011 | | 0.059 |
| 1012 | | 0.354 |
| 1013 | | 0.277 |
| 1014 | | 0.155 |
| 1015 | | 0.705 |
| 1016 | | |
| 1017 | | 0.226 |
| 1018 | | 0.065 |
| 1019 | | 0.898 |
| 1020 | | 0.264 |
| 1021 | | >10 |
| 1022 | | 0.726 |
| 1023 | | 0.547 |
| 1024 | | 0.236 |
| 1025 | | 0.141 |
| 1026 | | |
| 1027 | | |
| 1028 | | 0.087 |
| 1029 | | 0.49 |
| 1030 | | 1.11 |
| 1031 | | 0.085 |
| 1032 | | 1.44 |
| 1033 | | 1.52 |
| 1034 | | 1.67 |
| 1035 | | 1.35 |
| 1036 | | 7.64 |
| 1037 | | 3.81 |
| 1038 | | 0.331 |
| 1039 | | 0.244 |
| 1040 | | 0.217 |
| 1041 | | 0.25 |
| 1042 | | 0.093 |
| 1043 | | 0.235 |
| 1044 | | 1.34 |
| 1045 | | |
| 1046 | | 3.44 |
| 1047 | | 0.234 |
| 1048 | | 0.123 |
| 1049 | | 0.231 |
| 1050 | | 0.66 |
| 1051 | | 0.135 |
| 1052 | | 0.155 |
| 1053 | | 0.19 |
| 1054 | | 0.717 |
| 1055 | | 0.096 |
| 1056 | | 0.294 |
| 1057 | | 0.081 |
| 1058 | | 0.146 |
| 1059 | | 0.381 |
| 1060 | | 0.698 |
| 1061 | | 0.078 |
| 1062 | | 0.034 |
| 1063 | | 0.455 |
| 1064 | | 0.193 |
| 1065 | | 0.167 |
| 1066 | | 0.615 |
| 1067 | | 0.092 |
| 1068 | | 0.271 |
| 1069 | | 0.122 |
| 1070 | | 0.463 |
| 1071 | | 0.276 |
| 1072 | | 0.035 |
| 1074 | | 0.306 |
| 1075 | | 0.114 |
| 1076 | | 0.376 |
| 1077 | | 0.834 |
| 1078 | | 0.353 |
| 1079 | | 0.329 |
| 1080 | | 0.386 |
| 1081 | | 0.306 |
| 1082 | | 0.289 |
| 1083 | | 0.366 |
| 1084 | | 0.722 |
| 1085 | | 0.746 |
| 1086 | | 0.492 |
| 1087 | | 0.746 |
| 1088 | | 0.317 |
| 1089 | | 0.454 |
| 1090 | | 0.393 |
| 1091 | | 0.086 |
| 1092 | | 0.162 |
| 1093 | | 0.226 |
| 1094 | 10.4 | |
| 1095 | | 0.374 |

The entire disclosure of each patent, patent application, and publication cited or described in this document is hereby incorporated by reference.

We claim:

1. A compound of Formula (1): 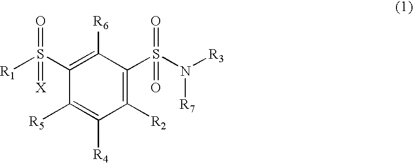
or a pharmaceutically acceptable salt thereof,
wherein
R₁ is 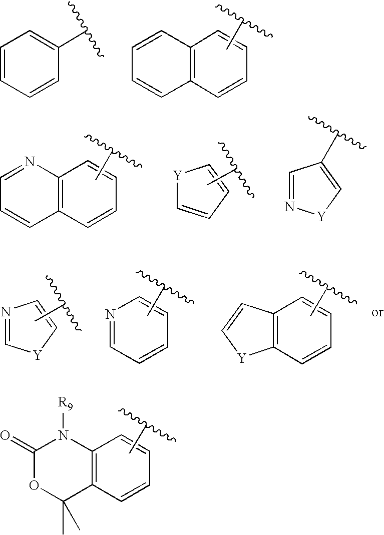
and each R₁ group is optionally substituted with up to three R₈ groups;
Y is O, S, or NR_{9;}
R₈ is alkyl, arylalkyl, perfluoroalkyl, alkenyl, arylalkenyl, alkynyl, arylalkynyl, cycloalkyl, alkylcycloalkyl, heterocycloalkyl, alkylheterocycloalkyl, aryl, alkylaryl, heteroaryl, alkoxy, perfluoroalkoxy, arylalkoxy, alkylcarbonyl, arylcarbonyl, halogen, cyano, azido, hydroxyl, carboxy, alkoxycarbonyl, alkylamino, dialkylamino, alkylaminocarbonyl, dialkylaminocarbonyl, alkylcarbonylamino, alkylcarbonylalkylamino, hydroxyalkylamino, nitro, alkylcarbonyloxime, alkylsulfonyl, alkylsulfinyl, alkylthio, perfluoroalkylthio, arylthio, tertiary alkylcarbinol, tertiary alkylcycloalkylcarbinol, or tertiary arylalkylcarbinol;
R₉ is hydrogen, alkyl, aryl, arylalkyl, cycloalkylalkyl, heterocycloalkyl, or spirocycloalkyl;
X is oxygen or an electron pair;
R₂ is hydrogen, alkyl, alkoxy, cycloalkyl, perfluoroalkyl, perfluoroalkylalkyl, perfluoroalkoxy, dialkylamino, or halogen;
R₄ is hydrogen, halogen, alkyl, cycloalkyl, alkoxy, perfluoroalkyl, or perfluoroalkoxy;
or R₂ and R₄, together with the carbon atoms to which they are attached, form a cycloalkyl ring of 5 to 7 carbon atoms that is optionally substituted with 1 to 3 R groups;
each R is, independently, hydrogen, alkyl, arylalkyl, cyanoalkyl, cycloalkyl, cycloalkylalkyl, heterocycloalkyl, spirocycloalkyl, aryl, arylalkyl, or alkoxyalkyl;
R₅, and R₆ are, independently, hydrogen, alkyl, aryl, alkoxy, halogen, or perfluoroalkyl;
R₃ and R₇ are each, independently, hydrogen or an optionally substituted alkyl, cycloalkyl, heterocycloalkyl, alkylheterocycloalkyl, heteroarylalkyl, alkylaryl, alkylheteroaryl, alkenyl, alkynyl, fused cycloalkylaryl, fused heterocycloalkylaryl, cycloalkylcarbonyl, or heterocycloalkylcarbonyl group;
or R₃ and R₇, together with the nitrogen atom to which they are attached, form a five or six membered heterocycloalkyl ring optionally substituted with 1 to 5 substituents selected from alkyl, aryl, heterocycloalkyl, heterocycloalkylalkyl, alkylamino, dialkylamino, alkoxycarbonyl, alkylcarbonyl, alkylaminocarbonylalkyl, and heterocycloalkylcarbonylalkyl;
provided that the compound is not
2-methyl-N-(2-phenylethyl)-5-(phenylsulfonyl)-benzenesulfonamide;
2-methyl-N-(2-phenylmethyl)-5-(phenylsulfonyl)-benzenesulfonamide;
5-[(4-chlorophenyl)sulfonyl]-N-cyclohexyl-2-methylbenzenesulfonamide;
N-benzyl-5-[(4-chlorophenyl)sulfonyl]-2-methylbenzenesulfonamide;
5-[(4-chlorophenyl)sulfonyl]-N-(2-furylmethyl)-2-methylbenzenesulfonamide;
5-[(4-chlorophenyl)sulfonyl]-2-methylbenzenesulfonamide;
2-methyl-5-(phenylsulfonyl)-benzenesulfonamide;
5-[(4-bromophenyl)sulfonyl]-2-methylbenzenesulfonamide;
2-methyl-5-[(4-nitrophenyl)sulfonyl]-benzenesulfonamide;
5-[(2,4-dinitrophenyl)sulfonyl]-2-methyl-benzenesulfonamide; or
5-[(4-chlorophenyl)sulfonyl]-2-methyl-N-(3-pyridinylmethyl)-benzenesulfonamide.

2. The compound of claim 1 wherein the alkyl, cycloalkyl, alkylheterocycloalkyl, heteroarylalkyl, alkylaryl, alkylheteroaryl, alkenyl, alkynyl, fused cycloalkylaryl, fused heterocycloalkylaryl, cycloalkylcarbonyl, and heterocycloalkylcarbonyl groups groups of R₃ and R₇ are each independently, optionally substituted with 1 to 5 substituents selected from alkyl, perfluoroalkyl, cycloalkyl, heterocycloalkyl, aryl, heteroaryl, fused cycloalkylaryl, alkoxy, aminocarbonylalkoxy, alkoxycarbonylalkoxy, carboxyalkoxy, cycloalkyloxy, aryloxy, amino, alkylamino, dialkylamino, alkoxycarbonylamino, carboxy, cyano, halogen, oxo, hydroxyl, alkylcarbonyl, carboxyalkylcarbonyl, arylaminocarbonyl, heterocycloalkylcarbonyl, alkoxycarbonyl, alkylaminocarbonyl, dialkylaminocarbonyl, fused cycloalkylarylaminocarbonyl, and fused heterocycloalkylarylcarbonyl.

3. The compound of claim 2 wherein the cycloalkyl, heterocycloalkyl, aryl, and heteroaryl substituents on the alkyl groups of R₃ and R₇ are independently, optionally substituted with 1 to 5 substituents selected from alkyl, cycloalkyl, heterocycloalkyl, spirocycloalkyl, perfluoroalkyl, haloalkyl, cyanoalkyl, carboxyalkyl, dimethylphosphonatealkyl, phosphonic acid alkyl, arylalkyl, cycloalkylalkyl, alkoxy, perfluoroalkoxy, arylalkoxy, benzoxy, aryl, heteroaryl, carboxyaryl, arylcarbonyl, alkylcarbonyl, perfluoroalkylcarbonyl, alkoxycarbonyl, carboxyalkylcarbonyl, aryloxycarbonyl, alkoxythiocarbonyl, aryloxythiocarbonyl, arylcarbonyl, cycloalkylcarbonyl, heterocycloalkylcarbonyl, heterocycloalkylthiocarbonyl, arylthiocarbonyl, alkylaminocarbonyl, dialkylaminocarbonyl, dialkylaminoarylcarbonyl, dialkylaminoalkylcarbonyl, alkylthiocarbonyl, arylaminocarbonyl, heteroarylcarbonyl, heteroarylaminocarbonyl, alkylaminothiocarbonyl, dialkylaminothiocarbonyl, arylaminothiocarbonyl, heteroarylaminothiocarbonyl, aminosulfonyl, alkylsulfonyl, arylsulfonyl, arylsulfonylarylsulfonyl, carboxyarylsulfonyl, nitro, amino, dialkylamino, alkylcarbonylamino, alkylsulfonylamino, carboxyarylsulfonylamino, hydroxy, carboxy, sulfonamide, alkylthio, halogen, cyano, guanidine, and oxo, and the nitrogen atoms in the heteroaryl substituents are optionally substituted with an oxygen atom.

4. The compound of claim 1 wherein the heterocycloalkyl groups of R₃ and R₇ are independently, optionally substituted with 1 to 5 substituents selected from alkyl, hydroxyalkyl, cyanoalkyl, carboxyalkyl, aminocarbonylalkyl, alkoxycarbonylalkyl, aminocarbonylalkyl, alkylaminocarbonylalkyl, dialkylaminocarbonylalkyl, heterocycloalkyl, heterocycloalkylalkyl, heterocycloalkylcarbonylalkyl, arylalkyl, heteroarylalkyl, arylcarbonylalkyl, alkylcarbonyl, cyano, alkylester, alkylamide, cycloalkylamide, aryl, arylester, alkylcarbonyl, perfluoroalkylcarbonyl, aminocarbonyl, arylaminocarbonyl, arylaminothiocarbonyl, cyanoalkoxycarbonyl, cycloalkylcarbonyl, arylcarbonyl, arylthiocarbonyl, alkylaminocarbonyl, dialkylaminocarbonyl, arylaminocarbonyl, alkylaminothiocarbonyl, dialkylaminothiocarbonyl, heteroarylcarbonyl, heterocycloalkylcarbonyl, cyanoarylcarbonyl, arylalkylcarbonyl, alkoxycarbonyl, alkoxyalkylcarbonyl, alkylthioalkylcarbonyl, alkylaminoalkylcarbonyl, dialkylaminoalkylcarbonyl, heterocycloalkylalkylcarbonyl, heterocycloalkylalkylaminothiocarbonyl, arylaminothiocarbonyl, heteroarylaminothiocarbonyl, heteroarylatkylcarbonyl, carboxyalkylcarbonyl, alkoxycarbonylaminothiocarbonyl, alkoxycarbonylalkylaminothiocarbonyl, alkylthiocarbonylalkylcarbonyl, alkylsulfonyl, arylsulfonyl, alkylaminoarylsulfonyl, and heteroarylsulfonyl.

5. The compound of claim 4 wherein the cycloalkyl, heterocycloalkyl, aryl, and heteroaryl substituents on the heterocycloalkyl groups of R₃ and R₇ are independently, optionally substituted with 1 to 5 substituents selected from alkyl, cycloalkyl, heterocycloalkyl, spirocycloalkyl, perfluoroalkyl, haloalkyl, cyanoalkyl, carboxyalkyl, dimethylphosphonatealkyl, phosphonic acid alkyl, arylalkyl, cycloalkylalkyl, alkoxy, perfluoroalkoxy, arylalkoxy, benzoxy, aryl, heteroaryl, carboxyaryl, arylcarbonyl, alkylcarbonyl, perfluoroalkylcarbonyl, alkoxycarbonyl, carboxyalkylcarbonyl, aryloxycarbonyl, alkoxythiocarbonyl, aryloxythiocarbonyl, arylcarbonyl, cycloalkylcarbonyl, heterocycloalkylcarbonyl, heterocycloalkylthiocarbonyl, arylthiocarbonyl, alkylaminocarbonyl, dialkylaminocarbonyl, dialkylaminoarylcarbonyl, dialkylaminoalkylcarbonyl, alkylthiocarbonyl, arylaminocarbonyl, heteroarylcarbonyl, heteroarylaminocarbonyl, alkylaminothiocarbonyl, dialkylaminothiocarbonyl, arylaminothiocarbonyl, heteroarylaminothiocarbonyl, aminosulfonyl, alkylsulfonyl, arylsulfonyl, arylsulfonylarylsulfonyl, carboxyarylsulfonyl, nitro, amino, dialkylamino, alkylcarbonylamino, alkylsulfonylamino, carboxyarylsulfonylamino, hydroxy, carboxy, sulfonamide, alkylthio, halogen, cyano, guanidine, and oxo, and the nitrogen atoms in the heteroaryl substituents are optionally substituted with an oxygen atom.

6. The compound of claim 2 wherein the amino substituents on the alkyl groups of R₃ and R₇ are independently, optionally substituted with 1 or 2 substituents selected from alkyl, hydroxyalkyl, carboxyalkyl, cycloalkyl, alkoxycarbonylalkyl, aryl, alkylcarbonyl, arylcarbonyl, alkylsulfonyl, arylsulfonyl, alkylcarbonyl, cycloalkylcarbonyl, alkylaminocarbonyl, dialkylaminocarbonyl, alkylaminothiocarbonyl, dialkylaminothiocarbonyl, alkoxycarbonylalkylaminocarbonyl, carboxyalkylcarbonyl, carboxyalkylaminocarbonyl, carboxyalkylcarbonylheterocycloalkylaminocarbonyl, arylaminocarbonyl, arylcarbonyl, heteroarylaminocarbonyl, heterocycloalkylcarbonyl, arylaminothiocarbonyl, heteroarylaminothiocarbonyl, heterocycloalkylaminocarbonyl, heterocycloalkylthiocarbonyl, heteroarylcarbonyl, alkoxycarbonyl, aryloxycarbonyl, alkoxythiocarbonyl, and aryloxythiocarbonyl.

7. The compound of claim 6 wherein the cycloalkyl, heterocycloalkyl, aryl, and heteroaryl substituents on the amino substituents on the alkyl groups of R₃ and R₇ are independently, optionally substituted with 1 to 5 substituents selected from alkyl, cycloalkyl, heterocycloalkyl, spirocycloalkyl, perfluoroalkyl, haloalkyl, cyanoalkyl, carboxyalkyl, dimethylphosphonatealkyl, phosphonic acid alkyl, arylalkyl, cycloalkylalkyl, alkoxy, perfluoroalkoxy, arylalkoxy, benzoxy, aryl, heteroaryl, carboxyaryl, arylcarbonyl, alkylcarbonyl, perfluoroalkylcarbonyl, alkoxycarbonyl, carboxyalkylcarbonyl, aryloxycarbonyl, alkoxythiocarbonyl, aryloxythiocarbonyl, arylcarbonyl, cycloalkylcarbonyl, heterocycloalkylcarbonyl, heterocycloalkylthiocarbonyl, arylthiocarbonyl, alkylaminocarbonyl, dialkylaminocarbonyl, dialkylaminoarylcarbonyl, dialkylaminoalkylcarbonyl, alkylthiocarbonyl, arylaminocarbonyl, heteroarylcarbonyl, heteroarylaminocarbonyl, alkylaminothiocarbonyl, dialkylaminothiocarbonyl, arylaminothiocarbonyl, heteroarylaminothiocarbonyl, aminosulfonyl, alkylsulfonyl, arylsulfonyl, arylsulfonylarylsulfonyl, carboxyarylsulfonyl, nitro, amino, dialkylamino, alkylcarbonylamino, alkylsulfonylamino, carboxyarylsulfonylamino, hydroxy, carboxy, sulfonamide, alkylthio, halogen, cyano, guanidine, and oxo, and the nitrogen atoms in the heteroaryl substituents are optionally substituted with an oxygen atom.

8. The compound of claim 1 wherein the alkyl group substituents on the heterocycloalkyl ring formed from R₃ and R_{7,} together with the nitrogen atom to which they are attached are each independently, optionally substituted with 1 to 5 substituents selected from aryl, heteroaryl optionally substituted with one to three alkyl groups, aminoalkyl, heterocycloalkyl, fused heterocycloalkylaryl, and heterocycloalkylcarbonyl.

9. The compound of claim 1 wherein R₁ is aryl.

10. The compound of claim 1 wherein each R₈ is, independently, alkyl, alkylaryl, alkylheteroaryl, alkylamino, dialkylamino, carboxy, alkylcarbonyl, alkoxy, perfluoroalkoxy, halogen, or cyano.

11. The compound of claim 1 wherein X is oxygen.

12. The compound of claim 1 wherein R_{4,} R₅, and R₆ are each hydrogen.

13. The compound of claim 1 wherein R₄ is methyl and R₅ and R₆ are each hydrogen, or R₅ is methyl and R₄ and R₆ are each hydrogen, or R₆ is methyl and R₄ and R₅ are each hydrogen.

14. The compound of claim 1 wherein R₂ is methyl, ethyl, isopropyl, propyl, Cl, methoxy, trifluoromethyl, or trifluoromethoxy.

15. The compound of claim 14 wherein R₂ is methyl, isopropyl, trifluoromethyl, or trifluoromethoxy.

16. The compound of claim 15 wherein R₂ is isopropyl or trifluoromethyl.

17. The compound of claim 1 wherein R₃ and R₇, together with the nitrogen atoms to which they are attached, form an optionally substituted 5 or 6 membered heterocycloalkyl group.

18. The compound of claim 17 wherein R₃ and R₇, together with the nitrogen atoms to which they are attached, form an optionally substituted piperazinyl group.

19. The compound of claim 1 wherein R₇ is hydrogen and R₃ is alkyl, cycloalkyl, 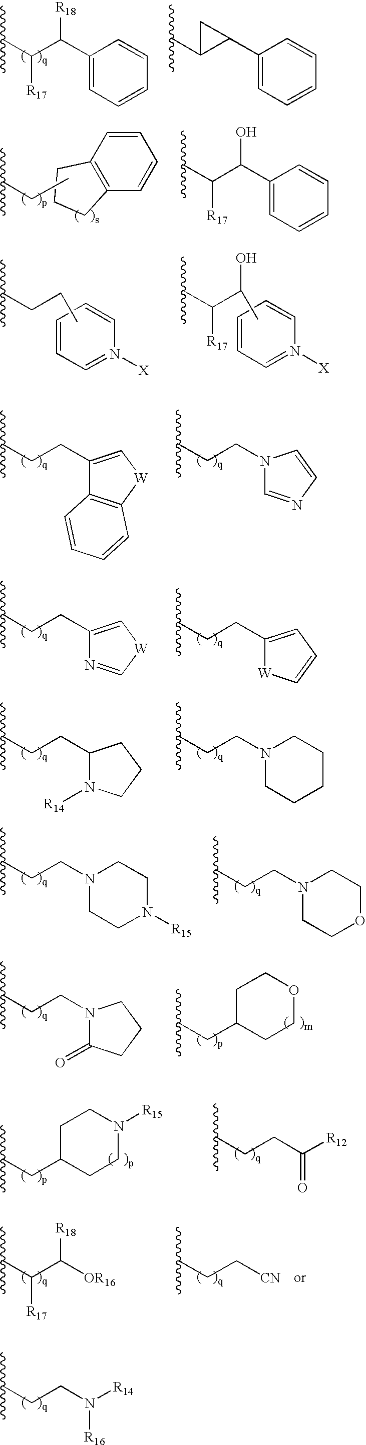
wherein the carbon atoms of each alkyl, cycloalkyl, heterocycloalkyl, aryl or heteroaryl group are optionally substituted with up to four R₁₃ groups;
R₁₃ is hydrogen, F, Cl, Br, alkyl, alkoxy, aryl, nitro, aminosulfonyl, arylalkoxy, perfluoroalkyl, perfluoroalkoxy, amino, alkylamino, dialkylamino, hydroxy, carboxy, cycloalkyl, carboxyalkyl, carboxyalkoxy, alkoxycarbonyl, aminocarbonylalkyl, alkoxycarbonylalkoxy, aminocarbonylalkoxy, alkylaminocarbonylalkyl, aminocarbonyl, alkylaminocarbonyl, dialkylaminocarbonyl, heterocycloalkylcarbonyl, heterocycloalkylaminocarbonyl, alkylaminocarbonylalkoxy, dialkylaminocarbonylalkyl, dialkylaminocarbonylalkoxy, heterocycloalkylcarbonylalkyl, heterocycloalkylaminocarbonylalkoxy, heterocycloalkylcarbonylalkoxy, cycloalkylaminoalkyl, heterocycloalkylaminoalkyl, aminoalkylaminoalkylarylamionalkyl, heteroarylaminoalkylarylalkylaminoalkyl, heteroarylalkylaminoalkyl, alkylaminoalkylamino, dialkylaminoalkylaminoarylamino, heteroarylaminoarylalkylamino, heteroarylalkylamino, or cyano;
each R₁₂ is alkyl, aryl, alkylamino, dialkylamino, alkoxy, hydroxyl, amino, arylamino, diarylamino, aryl(alkyl)amino, or aryloxy;
each R₁₄ is hydrogen, alkyl, aryl, cycloalkyl, alkylcarbonyl, arylcarbonyl, aryloxycarbonyl, alkylsulfonyl, arylsulfonyl, alkylaminocarbonyl, dialkylaminocarbonyl, alkylaminothiocarbonyl, dialkylaminothiocarbonyl, arylaminocarbonyl, heteroarylaminocarbonyl, heterocycloalkylcarbonyl, arylaminothiocarbonyl, heteroarylaminothiocarbonyl, heterocycloalkylthiocarbonyl, heteroarylcarbonyl, alkoxycarbonyl, aryloxycarbonyl, alkoxythiocarbonyl, alkoxycarbonylaminothiocarbonyl, cycloalkylcarbonyl, aminocarbonyl, alkoxycarbonyl, cycloalkylaminocarbonyl, heterocycloalkylaminocarbonyl, cycloalkylsulfonyl, heterocycloalkylsulfonyl, heteroarylsulfonyl, or aryloxythiocarbonyl;
R₅ is hydrogen, alkyl, aryl, cycloalkyl, alkylcarbonyl, arylcarbonyl, alkylsulfonyl, arylsulfonyl, alkylcarbonyl, arylcarbonyl, alkylaminocarbonyl, dialkylaminocarbonyl, alkylaminothiocarbonyl, dialkylaminothiocarbonyl, arylaminocarbonyl, heteroarylaminocarbonyl, heterocycloalkylcarbonyl, arylaminothiocarbonyl, heteroarylaminothiocarbonyl, heterocycloalkylthiocarbonyl, heterocycloalkylalkylaminothiocarbonyl, heteroarylcarbonyl, alkoxycarbonyl, aryloxycarbonyl, arylthiocarbonyl, alkoxythiocarbonyl, or aryloxythiocarbonyl;
R₁₆, R₁₇ and R₁₈ are each, independently, hydrogen, alkyl, aryl or cycloalkyl;
m is 0, 1, or 2;
p is 0, 1, or2;
q is 1 or 2;
s is 1 or 2; and
W is NR₉, O, or S.

20. The compound of claim 19 wherein the alkyl, aryl and cycloalkyl groups of R₁₃, R₁₄ and R₁₅ are each, independently, optionally substituted with 1 to 5 substituents selected from alkyl, cycloalkyl, heterocycloalkyl, perfluoroalkyl, aryl, heteroaryl, alkoxy, aryloxy, cycloalkyloxy, amino, alkylamino, dialkylamino, carboxy, cyano, oxo, hydroxyl, alkylcarbonyl, alkoxycarbonyl, arylcarbonyl, alkoxycarbonylamino, alkylcarbonylamino, aminocarbonyl, alkylaminocarbonyl, dialkylaminocarbonyl, alkylthio, alkyloxothio, and alkoxycarbonylalkylamino.

21. The compound of claim 19 wherein the arylsulfonyl, arylcarbonyl and heteroarylcarbonyl groups of R₁₃, R₁₄ and R₁₅ are each, independently, optionally substituted with 1 to 5 substituents selected from hydrogen, halogen, hydroxyl, alkyl, cycloalkyl, perfluoroalkyl, aryl, alkoxy, heterocycloalkyl, heteroaryl, cycloalkyloxy, aryloxy, amino, alkylamino, dialkylamino, alkylthio, alkyloxothio, carboxy, cyano, oxo, alkylcarbonyl, arylcarbonyl,alkoxycarbonyl, alkylcarbonylamino, aminocarbonyl, alkylaminocarbonyl, and dialkylaminocarbonyl.

22. The compound of claim 19 wherein the heterocycloalkyl groups of R₁₃, R₁₄ and R₁₅ are each, independently, optionally substituted with 1 to 5 substituents selected from hydrogen, hydroxyl, alkyl, cycloalkyl, perfluoroalkyl, aryl, heterocycloalkyl, heteroaryl, heteroarylcarbonyl, heteroarylalkylcarbonyl, alkylcarbonyl, alkylsulfonyl, arylsulfonyl, alkoxycarbonyl, arylcarbonyl, heteroarylcarbonyl, alkylaminocarbonyl, dialkylaminocarbonyl, alkylaminoalkylcarbonyl, dialkylaminoalkylcarbonyl, alkylaminocarbonylalkyl, dialkylaminocarbonylalkyl, heterocycloalkylcarbonylalkyl, carboxyalkylcarbonyl, and arylaminocarbonyl.

23. The compound of claim 19 wherein the alkylcarbonyl groups of R₁₃, R₁₄ and R₁₅ are each, independently, optionally substituted with 1 to 5 substituents selected from amino, alkylamino, dialkylamino, cycloalkyl, heterocycloalkyl, perfluoroalkyl, aryl, heteroaryl, alkoxy, aryloxy, cycloalkyloxy, carboxy, cyano, oxo, hydroxyl, alkylcarbonyl, alkoxycarbonyl, arylcarbonyl, and alkoxycarbonylamino.

24. The compound of claim 19 wherein the amino groups of the alkylamino and hetercycloalkylamino groups of R₁₃, R₁₄ and R₁₅ are each, independently, optionally substituted with a substituent selected from hydrogen, alkyl, cycloalkyl, and aryl.

25. The compound of claim 1 wherein R₇ is hydrogen and R₃ is heteroarylethyl, heteroarylpropyl, arylethyl, heterocycloalkyl, heterocycloalkylethyl, heterocycloalkylpropyl, heterocycloalkylmethyl, heterocyclalkylamino, cycloalkyl, fused cycloalkylaryl, aminoalkyl, or alkoxyalkyl.

26. The compound of claim 25 wherein R₇ is hydrogen and R₃ is heteroarylethyl, heteroarylpropyl, heterocyclalkylamino, fused cycloalkylaryl, or phenylethyl.

27. The compound of claim 26 wherein R₇ is hydrogen and R₃ is pyridinylethyl, imidazolylethyl, imidazolylpropyl, heterocyclalkylamino, fused cycloalkylaryl, or phenylethyl.

28. The compound of claim 1 which is
N-(2-phenylethyl)-3-(phenylsulfonyl)benzenesulfonamide;
N-[2-(2-chlorophenyl)ethyl]-3-(phenylsulfonyl)benzenesulfonamide;
N-[2-(2-methoxyphenyl)ethyl]-3-(phenylsulfonyl)benzenesulfonamide;
N-[2-(3-methoxyphenyl)ethyl]-3-(phenylsulfonyl)benzenesulfonamide;
N-[2-(3,4-dimethoxyphenyl)ethyl]-3-(phenylsulfonyl)benzenesulfonamide;
N-[2-(4-bromophenyl)ethyl]-3-(phenylsulfonyl)benzenesulfonamide;
N-[2-(4-fluorophenyl)ethyl]-3-(phenylsulfonyl)benzenesulfonamide;
N-[2-(4-chlorophenyl)ethyl]-3-(phenylsulfonyl)benzenesulfonamide;
N-[2-(4-methoxyphenyl)ethyl]-3-(phenylsulfonyl)benzenesulfonamide;
N-[2-(4-methylphenyl)ethyl]-3-(phenylsulfonyl)benzenesulfonamide;
N-{2-[4-(aminosulfonyl)phenyl]ethyl}-3-(phenylsulfonyl)benzenesulfonamide;
N-{2-[3,4-bis(benzyloxy)phenyl]ethyl}-3-(phenylsulfonyl)benzenesulfonamide;
N-[2-(4-nitrophenyl)ethyl]-3-(phenylsulfonyl)benzenesulfonamide;
N-[2-(1,3-benzodioxol-5-yl)ethyl]-3-(phenylsulfonyl)benzenesulfonamide;
3-(phenylsulfonyl)-N-{2-[3-(trifluoromethyl)phenyl]ethyl}benzenesulfonamide;
N-[2-(3-chlorophenyl)ethyl]-3-(phenylsulfonyl)benzenesulfonamide;
N-[2-(2,4-dichlorophenyl)ethyl]-3-(phenylsulfonyl)benzenesulfonamide;
N-[2-(2,6-dichlorophenyl)ethyl]-3-(phenylsulfonyl)benzenesulfonamide;
N-[2-(2,5-dimethoxyphenyl)ethyl]-3-(phenylsulfonyl)benzenesulfonamide;
N-[2-(3,4-dichlorophenyl)ethyl]-3-(phenylsulfonyl)benzenesulfonamide;
N-[2-(3,5-dimethoxyphenyl)ethyl]-3-(phenylsulfonyl)benzenesulfonamide;
N-[2-(4-ethoxyphenyl)ethyl]-3-(phenylsulfonyl)benzenesulfonamide;
N-[2-(3-fluorophenyl)ethyl]-3-(phenylsulfonyl)benzenesulfonamide;
N-[2-(2-fluorophenyl)ethyl]-3-(phenylsulfonyl)benzenesulfonamide;
N-[2-(3-ethoxy-4-methoxyphenyl)ethyl]-3-(phenylsulfonyl)benzenesulfonamide;
N-[2-(4-ethoxy-3-methoxyphenyl)ethyl]-3-(phenylsulfonyl)benzenesulfonamide;
N-(4-methoxybenzyl)-3-(phenylsulfonyl)benzenesulfonamide;
N-[2-(2-bromo-4,5-dimethoxyphenyl)ethyl]-3-(phenylsulfonyl)benzenesulfonamide;
N-[2-(5-bromo-2-methoxyphenyl)ethyl]-3-(phenylsulfonyl)benzenesulfonamide;
N³-{[3-(phenylsulfonyl)phenyl]sulfonyl}-beta-alaninamide;
methyl N-{[3-(phenylsulfonyl)phenyl]sulfonyl}-beta-alaninate;
N-(2-cyanoethyl)-3-(phenylsulfonyl)benzenesulfonamide;
3-(phenylsulfonyl)-N-(2-pyridin-2-ylethyl)benzenesulfonamide;
N-(3-morpholin-4-ylpropyl)-3-(phenylsulfonyl)benzenesulfonamide;
N-[2-(1-methylpyrrolidin-2-yl)ethyl]-3-(phenylsulfonyl)benzenesulfonamide;
3-(phenylsulfonyl)-N-(2-pyridin-4-ylethyl)benzenesulfonamide;
3-(phenylsulfonyl)-N-(2-piperidin-1-ylethyl)benzenesulfonamide;
N-[2-(1H-imidazol-4-yl)ethyl]-3-(phenylsulfonyl)benzenesulfonamide;
N-[2-({[3-(phenylsulfonyl)phenyl]sulfonyl}amino)ethyl]acetamide;
N-(2-morpholin-4-ylethyl)-3-(phenylsulfonyl)benzenesulfonamide;
N-[3-(2-oxopyrrolidin-1-yl)propyl]-3-(phenylsulfonyl)benzenesulfonamide;
N-[2-(diethylamino)ethyl]-3-(phenylsulfonyl)benzenesulfonamide;
N-[2-(dimethylamino)ethyl]-3-(phenylsulfonyl)benzenesulfonamide;
N-(3-methoxypropyl)-3-(phenylsulfonyl)benzenesulfonamide;
N-[3-(dimethylamino)propyl]-3-(phenylsulfonyl)benzenesulfonamide;
N-(2-methoxyethyl)-3-(phenylsulfonyl)benzenesulfonamide;
N-[3-(diethylamino)propyl]-3-(phenylsulfonyl)benzenesulfonamide;
N-[3-(1H-imidazol-1-yl)propyl]-3-(phenylsulfonyl)benzenesulfonamide;
3-(phenylsulfonyl)-N-(3-pyrrolidin-1-ylpropyl)benzenesulfonamide;
N-[3-(4-methylpiperazin-1-yl)propyl]-3-(phenylsulfonyl)benzenesulfonamide;
N-{[(3-(phenylsulfonyl)phenyl]sulfonyl}-beta-alanine;
N-2,3-dihydro-1H-inden-2-yl-3-(phenylsulfonyl)benzenesulfonamide;
N-[(1S,2R)-2-phenylcyclopropyl]-3-(phenylsulfonyl)benzenesulfonamide;
N-[(2R)-2-phenylpropyl]-3-(phenylsulfonyl)benzenesulfonamide; or
N-[(2S)-2-phenylpropyl]-3-(phenylsulfonyl)benzenesulfonamide.

29. The compound of claim 1 which is
5-[(4-fluorophenyl)sulfonyl]-2-methyl-N-(2-phenylethyl)benzenesulfonamide;
N-[2-(2-chlorophenyl)ethyl]-5-[(4-fluorophenyl)sulfonyl]-2-methylbenzenesulfonamide;
5-[(4-fluorophenyl)sulfonyl]-N-[2-(2-methoxyphenyl)ethyl]-2-methylbenzenesulfonamide;
5-[(4-fluorophenyl)sulfonyl]-N-[2-(3-methoxyphenyl)ethyl]-2-methylbenzenesulfonamide;
N-[2-(3,4-dimethoxyphenyl)ethyl]-5-[(4-fluorophenyl)sulfonyl]-2-methylbenzenesulfonamide;
N-[2-(4-bromophenyl)ethyl]-5-[(4-fluorophenyl)sulfonyl]-2-methylbenzenesulfonamide;
N-[2-(4-fluorophenyl)ethyl]-5-[(4-fluorophenyl)sulfonyl]-2-methylbenzenesulfonamide;
N-[2-(4-chlorophenyl)ethyl]-5-[(4-fluorophenyl)sulfonyl]-2-methylbenzenesulfonamide;
5-[(4-fluorophenyl)sulfonyl]-N-[2-(4-methoxyphenyl)ethyl]-2-methylbenzenesulfonamide;
5-[(4-fluorophenyl)sulfonyl]-2-methyl-N-[2-(4-methylphenyl)ethyl]benzenesulfonamide;
N-{2-[4-(aminosulfonyl)phenyl]ethyl}-5-[(4-fluorophenyl)sulfonyl]-2-methylbenzenesulfonamide;
N-{2-[3,4-bis(benzyloxy)phenyl]ethyl}-5-[(4-fluorophenyl)sulfonyl]-2-methylbenzenesulfonamide;
5-[(4-fluorophenyl)sulfonyl]-2-methyl-N-[2-(4-nitrophenyl)ethyl]benzenesulfonamide;
N-[2-(1,3-benzodioxol-5-yl)ethyl]-5-[(4-fluorophenyl)sulfonyl]-2-methylbenzenesulfonamide;
5-[(4-fluorophenyl)sulfonyl]-2-methyl-N-{2-[3-(trifluoromethyl)phenyl]ethyl}benzenesulfonamide;
N-[2-(3-chlorophenyl)ethyl]-5-[(4-fluorophenyl)sulfonyl]-2-methylbenzenesulfonamide;
N-[2-(2,4-dichlorophenyl)ethyl]-5-[(4-fluorophenyl)sulfonyl]-2-methylbenzenesulfonamide;
N-[2-(2,6-dichlorophenyl)ethyl]-5-[(4-fluorophenyl)sulfonyl]-2-methylbenzenesulfonamide;
N-[2-(2,5-dimethoxyphenyl)ethyl]-5-[(4-fluorophenyl)sulfonyl]-2-methylbenzenesulfonamide;
N-[2-(3,4-dichlorophenyl)ethyl]-5-[(4-fluorophenyl)sulfonyl]-2-methylbenzenesulfonamide;
N-[2-(3,5-dimethoxyphenyl)ethyl]-5-[(4-fluorophenyl)sulfonyl]-2-methylbenzenesulfonamide;
N-[2-(4-ethoxyphenyl)ethyl]-5-[(4-fluorophenyl)sulfonyl]-2-methylbenzenesulfonamide;
N-[2-(3-fluorophenyl)ethyl]-5-[(4-fluorophenyl)sulfonyl]-2-methylbenzenesulfonamide;
N-[2-(2-fluorophenyl)ethyl]-5-[(4-fluorophenyl)sulfonyl]-2-methylbenzenesulfonamide;
N-[2-(3-ethoxy-4-methoxyphenyl)ethyl]-5-[(4-fluorophenyl)sulfonyl]-2-methylbenzenesulfonamide;
N-[2-(4-ethoxy-3-methoxyphenyl)ethyl]-5-[(4-fluorophenyl)sulfonyl]-2-methylbenzenesulfonamide;
5-[(4-fluorophenyl)sulfonyl]-N-(4-methoxybenzyl)-2-methylbenzenesulfonamide;
N-[2-(2-bromo-4,5-dimethoxyphenyl)ethyl]-5-[(4-fluorophenyl)sulfonyl]-2-methylbenzenesulfonamide;
N-[2-(5-bromo-2-methoxyphenyl)ethyl]-5-[(4-fluorophenyl)sulfonyl]-2-methylbenzenesulfonamide;
2-Methyl-N-(2-phenylethyl)-5-(phenylsulfonyl)benzene sulfonamide;
N-[2-(2-chlorophenyl)ethyl]-2-methyl-5-(phenylsulfonyl)benzenesulfonamide;
N-[2-(2-methoxyphenyl)ethyl]-2-methyl-5-(phenylsulfonyl)benzenesulfonamide;
N-[2-(3-methoxyphenyl)ethyl]-2-methyl-5-(phenylsulfonyl)benzenesulfonamide;
N-[2-(3,4-dimethoxyphenyl)ethyl]-2-methyl-5-(phenylsulfonyl)benzenesulfonamide;
N-[2-(4-bromophenyl)ethyl]-2-methyl-5-(phenylsulfonyl)benzenesulfonamide;
N-[2-(4-fluorophenyl)ethyl]-2-methyl-5-(phenylsulfonyl)benzenesulfonamide;
N-[2-(4-chlorophenyl)ethyl]-2-methyl-5-(phenylsulfonyl)benzenesulfonamide;
N-[2-(4-methoxyphenyl)ethyl]-2-methyl-5-(phenylsulfonyl)benzenesulfonamide;
2-methyl-N-[2-(4-methylphenyl)ethyl]-5-(phenylsulfonyl)benzenesulfonamide;
N-{2-[4-(aminosulfonyl)phenyl]ethyl}-2-methyl-5-(phenylsulfonyl)benzenesulfonamide;
N-{2-[3,4-bis(benzyloxy)phenyl]ethyl}-2-methyl-5-(phenylsulfonyl)benzenesulfonamide;
2-methyl-N-[2-(4-nitrophenyl)ethyl]-5-(phenylsulfonyl)benzenesulfonamide;
N-[2-(1,3-benzodioxol-5-yl)ethyl]-2-methyl-5-(phenylsulfonyl)benzenesulfonamide;
2-methyl-5-(phenylsulfonyl)-N-{2-[3-(trifluoromethyl)phenyl]ethyl}benzenesulfonamide;
N-[2-(3-chlorophenyl)ethyl]-2-methyl-5-(phenylsulfonyl)benzenesulfonamide;
N-[2-(2,4-dichlorophenyl)ethyl]-2-methyl-5-(phenylsulfonyl)benzenesulfonamide;
N-[2-(2,6-dichlorophenyl)ethyl]-2-methyl-5-(phenylsulfonyl)benzenesulfonamide;
N-[2-(2,5-dimethoxyphenyl)ethyl]-2-methyl-5-(phenylsulfonyl)benzenesulfonamide;
N-[2-(3,4-dichlorophenyl)ethyl]-2-methyl-5-(phenylsulfonyl)benzenesulfonamide;
N-[2-(3,5-dimethoxyphenyl)ethyl]-2-methyl-5-(phenylsulfonyl)benzenesulfonamide;
N-[2-(4-ethoxyphenyl)ethyl]-2-methyl-5-(phenylsulfonyl)benzenesulfonamide;
N-[2-(3-fluorophenyl)ethyl]-2-methyl-5-(phenylsulfonyl)benzenesulfonamide;
N-[2-(2-fluorophenyl)ethyl]-2-methyl-5-(phenylsulfonyl)benzenesulfonamide;
N-[2-(3-ethoxy-4-methoxyphenyl)ethyl]-2-methyl-5-(phenylsulfonyl)benzenesulfonamide;
N-[2-(4-ethoxy-3-methoxyphenyl)ethyl]-2-methyl-5-(phenylsulfonyl)benzenesulfonamide;
N-(4-methoxybenzyl)-2-methyl-5-(phenylsulfonyl)benzenesulfonamide;
N-[2-(2-bromo-4,5-dimethoxyphenyl)ethyl]-2-methyl-5-(phenylsulfonyl)benzenesulfonamide;
N-[2-(5-bromo-2-methoxyphenyl)ethyl]-2-methyl-5-(phenylsulfonyl)benzenesulfonamide;
5-[(4-chlorophenyl)sulfonyl]-2-methyl-N-(2-phenylethyl)benzenesulfonamide;
N-[2-(2-chlorophenyl)ethyl]-5-[(4-chlorophenyl)sulfonyl]-2-methylbenzenesulfonamide;
5-[(4-chlorophenyl)sulfonyl]-N-[2-(2-methoxyphenyl)ethyl]-2-methylbenzenesulfonamide;
5-[(4-chlorophenyl)sulfonyl]-N-[2-(3-methoxyphenyl)ethyl]-2-methylbenzenesulfonamide;
5-[(4-chlorophenyl)sulfonyl]-N-[2-(3,4-dimethoxyphenyl)ethyl]-2-methylbenzenesulfonamide;
N-[2-(4-bromophenyl)ethyl]-5-[(4-chlorophenyl)sulfonyl]-2-methylbenzenesulfonamide;
5-[(4-chlorophenyl)sulfonyl]-N-[2-(4-fluorophenyl)ethyl]-2-methylbenzenesulfonamide;
N-[2-(4-chlorophenyl)ethyl]-5-[(4-chlorophenyl)sulfonyl]-2-methylbenzenesulfonamide;
5-[(4-chlorophenyl)sulfonyl]-N-[2-(4-methoxyphenyl)ethyl]-2-methylbenzenesulfonamide;
5-[(4-chlorophenyl)sulfonyl]-2-methyl-N-[2-(4-methylphenyl)ethyl]benzenesulfonamide;
N-{2-[4-(aminosulfonyl)phenyl]ethyl)}-5-[(4-chlorophenyl)sulfonyl]-2-methylbenzenesulfonamide;
5-[(4-chlorophenyl)sulfonyl]-2-methyl-N-[2-(4-nitrophenyl)ethyl]benzenesulfonamide;
N-[2-(1,3-benzodioxol-5-yl)ethyl]-5-[(4-chlorophenyl)sulfonyl]-2-methylbenzenesulfonamide;
5-[(4-chlorophenyl)sulfonyl]-2-methyl-N-{2-[3-(trifluoromethyl)phenyl]ethyl}benzenesulfonamide;
N-[2-(3-chlorophenyl)ethyl]-5-[(4-chlorophenyl)sulfonyl]-2-methylbenzenesulfonamide;
5-[(4-chlorophenyl)sulfonyl]-N-[2-(2,4-dichlorophenyl)ethyl]-2-methylbenzenesulfonamide;
5-[(4-chlorophenyl)sulfonyl]-N-[2-(2,6-dichlorophenyl)ethyl]-2-methylbenzenesulfonamide;
5-[(4-chlorophenyl)sulfonyl]-N-[2-(2,5-dimethoxyphenyl)ethyl]-2-methylbenzenesulfonamide;
5-[(4-chlorophenyl)sulfonyl]-N-[2-(3,4-dichlorophenyl)ethyl]-2-methylbenzenesulfonamide;
5-[(4-chlorophenyl)sulfonyl]-N-[2-(3,5-dimethoxyphenyl)ethyl]-2-methylbenzenesulfonamide;
5-[(4-chlorophenyl)sulfonyl]-N-[2-(4-ethoxyphenyl)ethyl]-2-methylbenzenesulfonamide;
5-[(4-chlorophenyl)sulfonyl]-N-[2-(3-fluorophenyl)ethyl]-2-methylbenzenesulfonamide;
5-[(4-chlorophenyl)sulfonyl]-N-[2-(2-fluorophenyl)ethyl]-2-methylbenzenesulfonamide;
5-[(4-chlorophenyl)sulfonyl]-N-[2-(3-ethoxy-4-methoxyphenyl)ethyl]-2-methylbenzenesulfonamide;
5-[(4-chlorophenyl)sulfonyl]-N-[2-(4-ethoxy-3-methoxyphenyl)ethyl]-2-methylbenzenesulfonamide;
5-[(4-chlorophenyl)sulfonyl]-N-(4-methoxybenzyl)-2-methylbenzenesulfonamide;
N-[2-(2-bromo-4,5-dimethoxyphenyl)ethyl]-5-[(4-chlorophenyl)sulfonyl]-2-methylbenzenesulfonamide;
N-[2-(5-bromo-2-methoxyphenyl)ethyl]-5-[(4-chlorophenyl)sulfonyl]-2-methylbenzenesulfonamide;
2-methyl-5-[(4-methylphenyl)sulfonyl]-N-(2-phenylethyl)benzenesulfonamide;
N-[2-(2-chlorophenyl)ethyl]-2-methyl-5-[(4-methylphenyl)sulfonyl]benzenesulfonamide;
N-[2-(2-methoxyphenyl)ethyl]-2-methyl-5-[(4-methylphenyl)sulfonyl]benzenesulfonamide;
N-[2-(3-methoxyphenyl)ethyl]-2-methyl-5-[(4-methylphenyl)sulfonyl]benzenesulfonamide;
N-[2-(3,4-dimethoxyphenyl)ethyl]-2-methyl-5-[(4-methylphenyl)sulfonyl]benzenesulfonamide;
N-[2-(4-bromophenyl)ethyl]-2-methyl-5-[(4-methylphenyl)sulfonyl]benzenesulfonamide;
N-[2-(4-fluorophenyl)ethyl]-2-methyl-5-[(4-methylphenyl)sulfonyl]benzenesulfonamide;
N-[2-(4-chlorophenyl)ethyl]-2-methyl-5-[(4-methylphenyl)sulfonyl]benzenesulfonamide;
N-[2-(4-methoxyphenyl)ethyl]-2-methyl-5-[(4-methylphenyl)sulfonyl]benzenesulfonamide;
2-methyl-N-[2-(4-methylphenyl)ethyl]-5-[(4-methylphenyl)sulfonyl]benzenesulfonamide;
N-{2-[4-(aminosulfonyl)phenyl]ethyl}-2-methyl-5-[(4-methylphenyl)sulfonyl]benzenesulfonamide;
2-methyl-5-[(4-methylphenyl)sulfonyl]-N-[2-(4-nitrophenyl)ethyl]benzenesulfonamide;
N-[2-(1,3-benzodioxol-5-yl)ethyl]-2-methyl-5-[(4-methylphenyl)sulfonyl]benzenesulfonamide;
2-methyl-5-[(4-methylphenyl)sulfonyl]-N-{2-[3-(trifluoromethyl)phenyl]ethyl}benzenesulfonamide;
N-[2-(3-chlorophenyl)ethyl]-2-methyl-5-[(4-methylphenyl)sulfonyl]benzenesulfonamide;
N-[2-(2,4-dichlorophenyl)ethyl]-2-methyl-5-[(4-methylphenyl)sulfonyl]benzenesulfonamide;
N-[2-(2,6-dichlorophenyl)ethyl]-2-methyl-5-[(4-methylphenyl)sulfonyl]benzenesulfonamide;
N-[2-(2,5-dimethoxyphenyl)ethyl]-2-methyl-5-[(4-methylphenyl)sulfonyl]benzenesulfonamide;
N-[2-(3,4-dichlorophenyl)ethyl]-2-methyl-5-[(4-methylphenyl)sulfonyl]benzenesulfonamide;
N-[2-(3,5-dimethoxyphenyl)ethyl]-2-methyl-5-[(4-methylphenyl)sulfonyl]benzenesulfonamide;
N-[2-(4-ethoxyphenyl)ethyl]-2-methyl-5-[(4-methylphenyl)sulfonyl]benzenesulfonamide;
N-[2-(3-fluorophenyl)ethyl]-2-methyl-5-[(4-methylphenyl)sulfonyl]benzenesulfonamide;
N-[2-(2-fluorophenyl)ethyl]-2-methyl-5-[(4-methylphenyl)sulfonyl]benzenesulfonamide;
N-[2-(2-bromo-4,5-dimethoxyphenyl)ethyl]-2-methyl-5-[(4-methylphenyl)sulfonyl]benzenesulfonamide;
N-[2-(5-bromo-2-methoxyphenyl)ethyl]-2-methyl-5-[(4-methylphenyl)sulfonyl]benzenesulfonamide;
2-ethyl-N-(2-phenylethyl)-5-(phenylsulfonyl)benzenesulfonamide;
2-ethyl-N-[2-(2-methoxyphenyl)ethyl]-5-(phenylsulfonyl)benzenesulfonamide;
2-ethyl-N-[2-(3-methoxyphenyl)ethyl]-5-(phenylsulfonyl)benzenesulfonamide;
N-[2-(3,4-dimethoxyphenyl)ethyl]-2-ethyl-5-(phenylsulfonyl)benzenesulfonamide;
N-{2-[4-(aminosulfonyl)phenyl]ethyl}-2-ethyl-5-(phenylsulfonyl)benzenesulfonamide;
2-ethyl-N-[2-(3-fluorophenyl)ethyl]-5-(phenylsulfonyl)benzenesulfonamide;
2-ethyl-N-[2-(2-fluorophenyl)ethyl]-5-(phenylsulfonyl)benzenesulfonamide;
N-[2-(3-ethoxy-4-methoxyphenyl)ethyl]-2-ethyl-5-(phenylsulfonyl)benzenesulfonamide;
N-[2-(4-ethoxy-3-methoxyphenyl)ethyl]-2-ethyl-5-(phenylsulfonyl)benzenesulfonamide;
2-methoxy-N-(2-phenylethyl)-5-(phenylsulfonyl)benzenesulfonamide;
2-methoxy-N-[2-(2-methoxyphenyl)ethyl]-5-(phenylsulfonyl)benzenesulfonamide;
2-methoxy-N-[2-(3-methoxyphenyl)ethyl]-5-(phenylsulfonyl)benzenesulfonamide;
N-[2-(3,4-dimethoxyphenyl)ethyl]-2-methoxy-5-(phenylsulfonyl)benzenesulfonamide;
N-{2-[4-(aminosulfonyl)phenyl]ethyl}-2-methoxy-5-(phenylsulfonyl)benzenesulfonamide;
N-[2-(3-fluorophenyl)ethyl]-2-methoxy-5-(phenylsulfonyl)benzenesulfonamide;
N-[2-(2-fluorophenyl)ethyl]-2-methoxy-5-(phenylsulfonyl)benzenesulfonamide;
N-[2-(3-ethoxy-4-methoxyphenyl)ethyl]-2-methoxy-5-(phenylsulfonyl)benzenesulfonamide;
N-[2-(4-ethoxy-3-methoxyphenyl)ethyl]-2-methoxy-5-(phenylsulfonyl)benzenesulfonamide;
2-methyl-N-(3-phenylpropyl)-5-(phenylsulfonyl)benzenesulfonamide;
2-ethyl-N-(3-phenylpropyl)-5-(phenylsulfonyl)benzenesulfonamide;
N-2,3-dihydro-1H-inden-2-yl-2-methyl-5-(phenylsulfonyl)benzenesulfonamide;
2-methyl-N-[(1S,2R)-2-phenylcyclopropyl]-5-(phenylsulfonyl)benzenesulfonamide;
2-methyl-N-[(2R)-2-phenylpropyl]-5-(phenylsulfonyl)benzenesulfonamide;
N-2,3-dihydro-1H-inden-2-yl-5-[(4-fluorophenyl)sulfonyl]-2-methylbenzenesulfonamide;
5-[(4-fluorophenyl)sulfonyl]-2-methyl-N-[(1S,2R)-2-phenylcyclopropyl]benzenesulfonamide;
5-[(4-fluorophenyl)sulfonyl]-2-methyl-N-[(2R)-2-phenylpropyl]benzenesulfonamide;
N-2,3-dihydro-1H-inden-2-yl-2-methyl-5-[(4-methylphenyl)sulfonyl]benzenesulfonamide;
2-methyl-5-[(4-methylphenyl)sulfonyl]-N-[(1S,2R)-2-phenylcyclopropyl]benzenesulfonamide;
2-methyl-5-[(4-methylphenyl)sulfonyl]-N-[(2R)-2-phenylpropyl]benzenesulfonamide;
5-[(4-chlorophenyl)sulfonyl]-N-2,3-dihydro-1H-inden-2-yl-2-methylbenzenesulfonamide;
5-[(4-chlorophenyl)sulfonyl]-2-methyl-N-[(1S,2R)-2-phenylcyclopropyl]benzenesulfonamide;
5-[(4-chlorophenyl)sulfonyl]-2-methyl-N-[(2R)-2-phenylpropyl]benzenesulfonamide;
5-[(4-fluorophenyl)sulfonyl]-2-methyl-N-[(2S)-2-phenylpropyl]benzenesulfonamide;
2-methyl-N-[(2S)-2-phenylpropyl]-5-(phenylsulfonyl)benzenesulfonamide;
2-methyl-5-[(4-methylphenyl)sulfonyl]-N-[(2S)-2-phenylpropyl]benzenesulfonamide;
5-[(4-chlorophenyl)sulfonyl]-2-methyl-N-[(2S)-2-phenylpropyl]benzenesulfonamide;
N-2,3-dihydro-1H-inden-2-yl-2-ethyl-5-(phenylsulfonyl)benzenesulfonamide;
2-ethyl-N-[(1S,2R)-2-phenylcyclopropyl]-5-(phenylsulfonyl)benzenesulfonamide;
2-ethyl-N-[(2R)-2-phenylpropyl]-5-(phenylsulfonyl)benzenesulfonamide;
N-2,3-dihydro-1H-inden-2-yl-2-methoxy-5-(phenylsulfonyl)benzenesulfonamide;
2-methoxy-N-[(1S,2R)-2-phenylcyclopropyl]-5-(phenylsulfonyl)benzenesulfonamide;
2-methoxy-N-[(2R)-2-phenylpropyl]-5-(phenylsulfonyl)benzenesulfonamide;
2-isopropyl-N-(2-phenylethyl)-5-(phenylsulfonyl)benzenesulfonamide;
2-methyl-N-(2-phenylethyl)-3-(phenylsulfonyl)benzenesulfonamide;
2-chloro-N-(2-phenylethyl)-5-(phenylsulfonyl)benzenesulfonamide;
2-(2-hydroxy-2-methylpropyl)-N-(2-phenylethyl)-5-(phenylsulfonyl)benzenesulfonamide;
N-{[2-methyl-5-(phenylsulfonyl)phenyl]sulfonyl}-L-phenylalaninamide;
methyl N-{[2-methyl-5-(phenylsulfonyl)phenyl]sulfonyl}-D-phenylalaninate;
N-(2,3-dihydro-1H-inden-1-yl)-2-methyl-5-(phenylsulfonyl)benzenesulfonamide;
N-[2-(2-fluorophenyl)ethyl]-3-[(4-methylphenyl)sulfonyl]-5,6,7,8-tetrahydronaphthalene-1-sulfonamide;
5-[(4-fluorophenyl)sulfonyl]-2-isopropyl-N-[(2R)-2-phenylpropyl]benzenesulfonamide;
5-[(4-fluorophenyl)sulfonyl]-2-isopropyl-N-[(2S)-2-phenylpropyl]benzenesulfonamide;
N-(2-phenylethyl)-5-(phenylsulfonyl)-2-propylbenzenesulfonamide;
5-(phenylsulfonyl)-2-propylbenzenesulfonamide;
5-[(4-fluorophenyl)sulfonyl]-N-(2-phenylethyl)-2-propylbenzenesulfonamide;
5-[(4-fluorophenyl)sulfonyl]-2-propylbenzenesulfonamide;
N-(2-phenylethyl)-5-(phenylsulfonyl)-2-(trifluoromethyl)-benzenesulfonamide;
2,4-diisopropyl-N-(2-phenylethyl)-5-(phenylsulfonyl)benzenesulfonamide;
N-(2,3-dihydro-1H-inden-2-yl)-2,4-diisopropyl-5-(phenylsulfonyl)benzenesulfonamide;
5-[(4-fluorophenyl)sulfonyl]-2,4-diisopropyl-N-(2-phenylethyl)benzenesulfonamide; or
N-(2,3-dihydro-1H-inden-2-yl)-5-[(4-fluorophenyl)sulfonyl]-2,4-diisopropylbenzenesulfonamide.

30. The compound of claim 1 which is
N-(2-hydroxy-2-phenylethyl)-2-methyl-5-(phenylsulfonyl)benzenesulfonamide;
5-[(4-fluorophenyl)sulfonyl]-N-(2-hydroxy-2-phenylethyl)-2-methylbenzenesulfonamide;
2-ethyl-5-[(4-fluorophenyl)sulfonyl]-N-(2-hydroxy-2-phenylethyl)benzenesulfonamide;
N-(2-hydroxy-2-phenylethyl)-2,4-dimethyl-5-(phenylsulfonyl)benzenesulfonamide;
trans-N-(2-hydroxy-1-methyl-2-phenylethyl)-2-methyl-5-(phenylsulfonyl)benzenesulfonamide;
5-[(4-fluorophenyl)sulfonyl]-N-[trans-2-hydroxy-1-methyl-2-phenylethyl]-2-methylbenzenesulfonamide; p1 2-ethyl-5-[(4-fluorophenyl)sulfonyl]-N-[(trans)-2-hydroxy-1-methyl-2-phenylethyl]benzenesulfonamide;
N-[(trans)-2-hydroxy-1-methyl-2-phenylethyl]-2,4-dimethyl-5-(phenylsulfonyl)benzenesulfonamide;
N-[(1S*,2S*)-2-hydroxycyclohexyl]-2-methyl-5-(phenylsulfonyl)benzenesulfonamide or
5-[(4-fluorophenyl)sulfonyl]-N-(2-hydroxyethyl)-2-isopropylbenzenesulfonamide.

31. The compound of claim 1 which is
5-[(4-fluorophenyl)sulfonyl]-2-methyl-N-(2-pyridin-2-ylethyl)benzenesulfonamide;
5-[(4-fluorophenyl)sulfonyl]-2-methyl-N-(3-morpholin-4-ylpropyl)benzenesulfonamide;
5-[(4-fluorophenyl)sulfonyl]-2-methyl-N-(2-pyridin-4-ylethyl)benzenesulfonamide;
2-methyl-5-(phenylsulfonyl)-N-(2-pyridin-2-ylethyl)benzenesulfonamide;
2-methyl-5-(phenylsulfonyl)-N-(2-pyridin-4-ylethyl)benzenesulfonamide;
2-ethyl-5-(phenylsulfonyl)-N-(2-pyridin-2-ylethyl)benzenesulfonamide;
2-methoxy-5-(phenylsulfonyl)-N-(2-pyridin-2-ylethyl)benzenesulfonamide;
2-methoxy-5-(phenylsulfonyl)-N-(2-pyridin-4-ylethyl)benzenesulfonamide;
5-[(4-chlorophenyl)sulfonyl]-2-methyl-N-(2-pyridin-2-ylethyl)benzenesulfonamide;
5-[(4-fluorophenyl)sulfonyl]-2-isopropyl-N-(2-pyridin-2-ylethyl)benzenesulfonamide;
2,4-dimethyl-5-(phenylsulfonyl)-N-(2-pyridin-2-ylethyl)benzenesulfonamide;
5-[(4-fluorophenyl)sulfonyl]-2,4-dimethyl-N-(2-pyridin-2-ylethyl)benzenesulfonamide;
2-chloro-5-(phenylsulfonyl)-N-(2-pyridin-2-ylethyl)benzenesulfonamide;
2,3-dimethyl-5-(phenylsulfonyl)-N-(2-pyridin-2-ylethyl)benzenesulfonamide;
2-methyl-5-{[4-(methylamino)phenyl]sulfonyl}-N-(2-pyridin-2-ylethyl)benzenesulfonamide;
2-isopropyl-5-{[4-(methylamino)phenyl]sulfonyl}-N-(2-pyridin-2-ylethyl)benzenesulfonamide;
5-{[4-(1-cyclohexyl-1-hydroxyethyl)phenyl]sulfonyl}-2-methyl-N-(2-pyridin-2-ylethyl)benzenesulfonamide;
5-{[4-(1-hydroxy-1-phenylethyl)phenyl]sulfonyl}-2-methyl-N-(2-pyridin-2-ylethyl)benzenesulfonamide;
5-{[4-(1-hydroxy-1-methyl-2-phenylethyl)phenyl]sulfonyl}-2-methyl-N-(2-pyridin-2-ylethyl)benzenesulfonamide;
5-[(3-cyanophenyl)sulfonyl]-2-methyl-N-(2-pyridin-2-ylethyl)benzenesulfonamide;
2-methyl-5-( 1-naphthylsulfonyl)-N-(2-pyridin-2-ylethyl)benzenesulfonamide;
5-[(3-hydroxyphenyl)sulfonyl]-2-methyl-N-(2-pyridin-2-ylethyl)benzenesulfonamide;
5-[(3,5-difluorophenyl)sulfonyl]-2-methyl-N-(2-pyridin-2-ylethyl)benzenesulfonamide;
5-[(4-ethylphenyl)sulfonyl]-2-methyl-N-(2-pyridin-2-ylethyl)benzenesulfonamide;
5-[(3-acetylphenyl)sulfonyl]-2-methyl-N-(2-pyridin-2-ylethyl)benzenesulfonamide;
5-[(2-ethoxyphenyl)sulfonyl]-2-methyl-N-(2-pyridin-2-ylethyl)benzenesulfonamide;
5-[(2,5-dimethoxyphenyl)sulfonyl]-2-methyl-N-(2-pyridin-2-ylethyl)benzenesulfonamide;
5-[(2,3-dimethoxyphenyl)sulfonyl]-2-methyl-N-(2-pyridin-2-ylethyl)benzenesulfonamide;
5-[(2,4-dimethoxyphenyl)sulfonyl]-2-methyl-N-(2-pyridin-2-ylethyl)benzenesulfonamide;
2-methyl-N-(2-pyridin-2-ylethyl)-5-(pyridin-3-ylsulfonyl)benzenesulfonamide;
5-(1H-indol-5-ylsulfonyl)-2-methyl-N-(2-pyridin-2-ylethyl)benzenesulfonamide;
5-[(4-cyanophenyl)sulfonyl]-2-methyl-N-(2-pyridin-2-ylethyl)benzenesulfonamide;
5-{[3-(ethylsulfonyl)phenyl]sulfonyl}-2-methyl-N-(2-pyridin-2-ylethyl)benzenesulfonamide;
2-methyl-5-[(2-methylphenyl)sulfonyl]-N-(2-pyridin-2-ylethyl)benzenesulfonamide;
5-[(2-ethylphenyl)sulfonyl]-2-methyl-N-(2-pyridin-2-ylethyl)benzenesulfonamide;
5-(biphenyl-2-ylsulfonyl)-2-methyl-N-(2-pyridin-2-ylethyl)benzenesulfonamide;
5-(biphenyl-4-ylsulfonyl)-2-methyl-N-(2-pyridin-2-ylethyl)benzenesulfonamide;
5-(biphenyl-3-ylsulfonyl)-2-methyl-N-(2-pyridin-2-ylethyl)benzenesulfonamide;
5-[(4-tert-butylphenyl)sulfonyl]-2-isopropyl-N-(2-pyridin-2-ylethyl)benzenesulfonamide;
5-[(4-tert-butylphenyl)sulfonyl]-2-methyl-N-(2-pyridin-2-ylethyl)benzenesulfonamide;
5-[(3,5-dimethylphenyl)sulfonyl]-2-isopropyl-N-(2-pyridin-2-ylethyl)benzenesulfonamide;
5-[(3-chlorophenyl)sulfonyl]-2-isopropyl-N-(2-pyridin-2-ylethyl)benzenesulfonamide;
2-isopropyl-5-[(3-methoxyphenyl)sulfonyl]-N-(2-pyridin-2-ylethyl)benzenesulfonamide;
2-isopropyl-5-[(2-methoxyphenyl)sulfonyl]-N-(2-pyridin-2-ylethyl)benzenesulfonamide;
5-[(3,5-difluorophenyl)sulfonyl]-2-isopropyl-N-(2-pyridin-2-ylethyl)benzenesulfonamide;
2-cyclohexyl-5-(phenylsulfonyl)-N-(2-pyridin-2-ylethyl)benzenesulfonamide;
2-cyclohexyl-5-[(4-fluorophenyl)sulfonyl]-N-(2-pyridin-2-ylethyl)benzenesulfonamide;
2-tert-butyl -5-(phenylsulfonyl)-N-(2-pyridin-2-ylethyl)benzenesulfonamide;
2,6-dimethyl-3-(phenylsulfonyl)-N-(2-pyridin-2-ylethyl)benzenesulfonamide;
5-{[5-(dimethylamino)-1-naphthyl]sulfonyl}-2-isopropyl-N-(2-pyridin-2-ylethyl)benzenesulfonamide;
5-[(3-chloro-5-cyanophenyl)sulfonyl]-2-methyl-N-(2-pyridin-2-ylethyl)benzenesulfonamide
2-methyl-3-(phenylsulfonyl)-N-(2-pyridin-2-ylethyl)benzenesulfonamide;
5-[(3,5-dichlorophenyl)sulfonyl]-2-isopropyl-N-(2-pyridin-2-ylethyl)benzenesulfonamide;
2-isopropyl-N-(2-pyridin-2-ylethyl)-5-{[3-(trifluoromethoxy)phenyl]sulfonyl}benzenesulfonamide;
5-{[4-(dimethylamino)phenyl]sulfonyl}-2-isopropyl-N-(2-pyridin-2-ylethyl)benzenesulfonamide;
2-isopropyl-N-(2-pyridin-2-ylethyl)-5-{[3-(trifluoromethyl)phenyl]sulfonyl}benzenesulfonamide;
5-[(5-chloro-2-methoxyphenyl)sulfonyl]-2-isopropyl-N-(2-pyridin-2-ylethyl)benzenesulfonamide;
2-isopropyl-N-(2-pyridin-2-ylethyl)-5-(quinolin-8-ylsulfonyl)benzenesulfonamide;
5-[(2,5-dichloro-3-thienyl)sulfonyl]-2-isopropyl-N-(2-pyridin-2-ylethyl)benzenesulfonamide;
5-[(5-chloro-1,3-dimethyl-1H-pyrazol-4-yl)sulfonyl]-2-isopropyl-N-(2-pyridin-2-ylethyl)benzenesulfonamide;
2-isopropyl-5-[(1-methyl-1H-imidazol-4-yl)sulfonyl]-N-(2-pyridin-2-ylethyl)benzenesulfonamide;
5-[(3-chloro-2-methylphenyl)sulfonyl]-2-isopropyl-N-(2-pyridin-2-ylethyl)benzenesulfonamide;
5-[(4,4-dimethyl-2-oxo-1,4-dihydro-2H-3,1-benzoxazin-6-yl)sulfonyl]-2-isopropyl-N-(2-pyridin-2-ylethyl)benzenesulfonamide;
5-[(4-fluorophenyl)sulfonyl]-2-isopropyl-N-(2-pyridin-4-ylethyl)benzenesulfonamide;
5-[(4-fluorophenyl)sulfonyl]-2-isopropyl-N-(2-pyridin-3-ylethyl)benzenesulfonamide;
5-[(3-cyanophenyl)sulfonyl]-2-isopropyl-N-(2-pyridin-2-ylethyl)benzenesulfonamide;
5-(1H-indol-5-ylsulfonyl)-2-isopropyl-N-(2-pyridin-2-ylethyl)benzenesulfonamide;
5-(phenylsulfonyl)-2-propyl-N-(2-pyridin-2-ylethyl)benzenesulfonamide;
5-[(4-fluorophenyl)sulfonyl]-2-propyl-N-(2-pyridin-2-ylethyl)benzenesulfonamide;
5-(phenylsulfonyl)-N-(2-pyridin-2-ylethyl)-2-(trifluoromethyl)benzenesulfonamide;
2-isopropyl-5-(phenylsulfinyl)-N-(2-pyridin-2-ylethyl)benzenesulfonamide;
5-[(3-bromo-2-methylphenyl)sulfonyl]-2-isopropyl-N-(2-pyridin-2-ylethyl)benzenesulfonamide;
5-[(2-chloro-6-methylphenyl)sulfonyl]-2-isopropyl-N-(2-pyridin-2-ylethyl)benzenesulfonamide;
2-isopropyl-5-[(3-methylphenyl)sulfonyl]-N-(2-pyridin-2-ylethyl)benzenesulfonamide;
2-isopropyl-5-[(R)-phenylsulfinyl]-N-(2-pyridin-2-ylethyl)benzenesulfonamide;
2-isopropyl-5-[(S)-phenylsulfinyl]-N-(2-pyridin-2-ylethyl)benzenesulfonamide;
2-bromo-5-(phenylsulfonyl)-N-(2-pyridin-2-ylethyl)benzenesulfonamide;
5-[(3-cyano-2-methylphenyl)sulfonyl]-2-isopropyl-N-(2-pyridin-2-ylethyl)benzenesulfonamide;
5-[(3-acetyl-2-methylphenyl)sulfonyl]-2-isopropyl-N-(2-pyridin-2-ylethyl)benzenesulfonamide;
5-[(3-chloro-4-fluorophenyl)sulfonyl]-2-isopropyl-N-(2-pyridin-2-ylethyl)benzenesulfonamide;
5-[(4-fluoro-2-methylphenyl)sulfonyl]-2-isopropyl-N-(2-pyridin-2-ylethyl)benzenesulfonamide;
2-isopropyl-5-[(1-methyl-1H-indol-5-yl)sulfonyl]-N-(2-pyridin-2-ylethyl)benzenesulfonamide;
2-isopropyl-5-{[2-methyl-4-(methylamino)phenyl]sulfonyl)}-N-(2-pyridin-2-ylethyl)benzenesulfonamide;
5-{[3-chloro-4-(methylamino)phenyl]sulfonyl}-2-isopropyl-N-(2-pyridin-2-ylethyl)benzenesulfonamide;
2,4-diisopropyl-5-(phenylsulfonyl)-N-(2-pyridin-2-ylethyl)benzenesulfonamide;
2,4-diisopropyl-5-(phenylsulfonyl)-N-(2-pyridin-3-ylethyl)benzenesulfonamide;
2,4-diisopropyl-5-(phenylsulfonyl)-N-(2-pyridin-4-ylethyl)benzenesulfonamide;
5-[(4-fluorophenyl)sulfonyl]-2,4-diisopropyl-N-(2-pyridin-2-ylethyl)benzenesulfonamide;
5-[(4-fluorophenyl)sulfonyl]-2,4-diisopropyl-N-(2-pyridin-3-ylethyl)benzenesulfonamide;
5-[(4-fluorophenyl)sulfonyl]-2,4-diisopropyl-N-(2-pyridin-4-ylethyl)benzenesulfonamide;
2-chloro-5-(phenylsulfonyl)-N-(2-pyridin-3-ylethyl)benzenesulfonamide;
2-chloro-5-(phenylsulfonyl)-N-(2-pyridin-4-ylethyl)benzenesulfonamide; or
5-[(4-fluorophenyl)sulfonyl]-2-isopropyl-N-[2-(1-oxidopyridin-3-yl)ethyl]benzenesulfonamide.

32. The compound of claim 1 which is
5-[(4-fluorophenyl)sulfonyl]-N-[2-(1H-imidazol-4-yl)ethyl]-2-methylbenzenesulfonamide;
5-[(4-fluorophenyl)sulfonyl]-N-[3-(1H-imidazol-1-yl)propyl]-2-methylbenzenesulfonamide;
N-[2-(1H-imidazol-4-yl)ethyl]-2-methyl-5-(phenylsulfonyl)benzenesulfonamide;
N-[3-(1H-imidazol-1-yl)propyl]-2-methyl-5-(phenylsulfonyl)benzenesulfonamide;
2-ethyl-N-[2-(1H-imidazol-4-yl)ethyl]-5-(phenylsulfonyl)benzenesulfonamide;
2-ethyl-N-[3-(1H-imidazol-1-yl)propyl]-5-(phenylsulfonyl)benzenesulfonamide;
N-[2-(1H-imidazol-4-yl)ethyl]-2-methoxy-5-(phenylsulfonyl)benzenesulfonamide;
N-[3-(1H-imidazol-1-yl)propyl]-2-methoxy-5-(phenylsulfonyl)benzenesulfonamide;
5-[(4-chlorophenyl)sulfonyl]-N-[3-(1H-imidazol-1-yl)propyl]-2-methylbenzenesulfonamide;
5-[(4-azidophenyl)sulfonyl]-N-[3-(1H-imidazol-1-yl)propyl]-2-methylbenzenesulfonamide;
N-[2-(1H-indol-3-yl)ethyl]-2-methyl-5-(phenylsulfonyl)benzenesulfonamide;
N-[3-(1H-imidazol-1-yl)propyl]-2-isopropyl-5-(phenylsulfonyl)benzenesulfonamide;
2-ethyl-5-[(4-fluorophenyl)sulfonyl]-N-[2-(1H-imidazol-1-yl)ethyl]benzenesulfonamide;
2-ethyl-5-[(4-fluorophenyl)sulfonyl]-N-[3-(1H-imidazol-1-yl)propyl]benzenesulfonamide;
5-({4-[ethyl(methyl)amino]phenyl}sulfonyl)-N-[3-(1H-imidazol-1-yl)propyl]-2-methylbenzenesulfonamide;
N-[3-(1H-imidazol-1-yl)propyl]-2-methyl-5-[(4-pyrrolidin-1-ylphenyl)sulfonyl]benzenesulfonamide;
N-[3-(1H-imidazol-1-yl)propyl]-4-methyl-3-(phenylsulfinyl)benzenesulfonamide;
5-[(4-fluorophenyl)sulfonyl]-N-[2-(1H-imidazol-1-yl)ethyl]-2-methylbenzenesulfonamide;
N-[3-(1H-imidazol-1-yl)propyl]-2-methyl-5-{[4-(methylamino)phenyl]sulfonyl}benzenesulfonamide;
5-{[4-(ethylamino)phenyl]sulfonyl}-N-[3-(1H-imidazol-1-yl)propyl]-2-methylbenzenesulfonamide;
N-[3-(1H-imidazol-1-yl)propyl]-2-methyl-5-[(4-piperidin-1-ylphenyl)sulfonyl]benzenesulfonamide;
N-[3-(1H-imidazol-1-yl)propyl]-2-methyl-5-[(4-morpholin-4-ylphenyl)sulfonyl]benzenesulfonamide;
N-[2-(1H-imidazol-1-yl)ethyl]-2-methyl-5-(phenylsulfonyl)benzenesulfonamide;
N-[3-(1H-imidazol-1-yl)propyl]-5-[(4-methoxyphenyl)sulfonyl]-2-methylbenzenesulfonamide;
N-[3-(1H-imidazol-1-yl)propyl]-5-[(2-methoxyphenyl)sulfonyl]-2-methylbenzenesulfonamide;
N-[3-(1H-imidazol-1-yl)propyl]4-methyl-3-(phenylsulfonyl)benzenesulfonamide;
5-[(4-fluorophenyl)sulfonyl]-N-[2-(1H-imidazol-1-yl)ethyl]-2-isopropylbenzenesulfonamide;
5-[(4-fluorophenyl)sulfonyl]-N-[3-(1H-imidazol-1-yl)propyl]-2-isopropylbenzenesulfonamide;
N-[3-(1H-imidazol-1-yl)propyl]-5-[(3-methoxyphenyl)sulfonyl]-2-methylbenzenesulfonamide;
N-[2-(1H-imidazol-1-yl)ethyl]-2,4-dimethyl-5-(phenylsulfonyl)benzenesulfonamide;
N-[3-(1H-imidazol-1-yl)propyl]-2,4-dimethyl-5-(phenylsulfonyl)benzenesulfonamide;
5-[(4-fluorophenyl)sulfonyl]-N-[2-(1H-imidazol-1-yl)ethyl]-2,4-dimethylbenzenesulfonamide;
5-[(4-fluorophenyl)sulfonyl]-N-[3-(1H-imidazol-1-yl)propyl]-2,4-dimethylbenzenesulfonamide;
2-chloro-N-[3-(1H-imidazol-1-yl)propyl]-5-(phenylsulfonyl)benzenesulfonamide;
N-[3-(1H-imidazol-1-yl)propyl]-5-[(4-isopropylphenyl)sulfonyl]-2-methylbenzenesulfonamide;
N-[3-(1H-imidazol-1-yl)propyl]-2-methyl-5-(2-naphthylsulfonyl)benzenesulfonamide;
5-[(3,4-dichlorophenyl)sulfonyl]-N-[3-(1H-imidazol-1-yl)propyl]-2-methylbenzenesulfonamide;
5-[(3-chlorophenyl)sulfonyl]-N-[3-(1H-imidazol-1-yl)propyl]-2-methylbenzenesulfonamide;
5-[(3,5-dimethylphenyl)sulfonyl]-N-[3-(1H-imidazol-1-yl)propyl]-2-methylbenzenesulfonamide;
5-[(3,5-dichlorophenyl)sulfonyl]-N-[3-(1H-imidazol-1-yl)propyl]-2-methylbenzenesulfonamide;
5-[(2,5-dichlorophenyl)sulfonyl]-N-[3-(1H-imidazol-1-yl)propyl]-2-methylbenzenesulfonamide;
N-[3-(1H-imidazol-1-yl)propyl]-2-methyl-5-(phenylsulfinyl)benzenesulfonamide;
N-[2-(1H-imidazol-1-yl)ethyl]-2,3-dimethyl-5-(phenylsulfonyl)benzenesulfonamide;
N-[3-(1H-imidazol-1-yl)propyl]-2,3-dimethyl-5-(phenylsulfonyl)benzenesulfonamide;
5-{[4-(cyclohexylamino)phenyl]sulfonyl}-N-[3-(1H-imidazol-1-yl)propyl]-2-methylbenzenesulfonamide;
5-({4-[(2-cyanoethyl)amino]phenyl}sulfonyl)-N-[3-(1H-imidazol-1-yl)propyl]-2-methylbenzenesulfonamide; p1 5-[(4-{[(1S,2S)-1-(hydroxymethyl)-2-methylbutyl]amino}phenyl)sulfonyl]-N-[3-(1H-imidazol-1-yl)propyl]-2-methylbenzenesulfonamide;
N-[3-(1H-imidazol-1-yl)propyl]-2-methyl-5-({4-[(1-phenylethyl)amino]phenyl}sulfonyl)benzenesulfonamide;
5-[(2,3-dichlorophenyl)sulfonyl]-N-[3-(1H-imidazol-1-yl)propyl]-2-methylbenzenesulfonamide;
N-[3-(1H-imidazol-1-yl)propyl]-2-methyl-5-(2-thienylsulfonyl)benzenesulfonamide;
N-[3-(1H-imidazol-1-yl)propyl]-2-methyl-5-[(2-methyl-3-furyl)sulfonyl]benzenesulfonamide;
N-[3-(1H-imidazol-1-yl)propyl]-2-methyl-5-{[4-(tetrahydro-2H-pyran-4-ylamino)phenyl]sulfonyl}benzenesulfonamide;
N-[3-(1H-imidazol-1-yl)propyl]-5-({4-[(3-isopropoxypropyl)amino]phenyl}sulfonyl)-2-methylbenzenesulfonamide;
5-({4-[(cyclopropylmethyl)amino]phenyl}sulfonyl)-N-[3-(1H-imidazol-1-yl)propyl]-2-methylbenzenesulfonamide;
5-({4-[(1R,2R,4S)-bicyclo[2.2.1]hept-2-ylamino]phenyl}sulfonyl)-N-[3-(1H-imidazol-1-yl)propyl]-2-methylbenzenesulfonamide;
5-{[4-(benzylamino)phenyl]sulfonyl}-N-[3-(1H-imidazol-1-yl)propyl]-2-methylbenzenesulfonamide;
5-[(4-{[(1S)-1-cyclohexylethyl]amino}phenyl)sulfonyl]-N-[3-(1H-imidazol-1-yl)propyl]-2-methylbenzenesulfonamide;
5-[(4-{[(1R)-1-cyclohexylethyl]amino}phenyl)sulfonyl]-N-[3-(1H-imidazol-1-yl)propyl]-2-methylbenzenesulfonamide;
5-({4-[(2-hydroxybutyl)amino]phenyl}sulfonyl)-N-[3-(1H-imidazol-1-yl)propyl]-2-methylbenzenesulfonamide;
N-[3-(1H-imidazol-1-yl)propyl]-2-methyl-5-[(4-{[4-(trifluoromethyl)benzyl]amino}phenyl)sulfonyl]benzenesulfonamide;
5-[(2-chlorophenyl)sulfonyl]-N-[3-(1H-imidazol-1-yl)propyl]-2-methylbenzenesulfonamide;
N-(tert-butyl)-2-methyl-5-(phenylsulfonyl)benzenesulfonamide;
5-[(4-tert-butylphenyl)sulfonyl]-N-[3-(1H-imidazol-1-yl)propyl]-2-isopropylbenzenesulfonamide;
5-[(4-tert-butylphenyl)sulfonyl]-N-[3-(1H-imidazol-1-yl)propyl]-2-methylbenzenesulfonamide;
5-[(4-fluorophenyl)sulfonyl]-N-[2-(1H-imidazol-4-yl)ethyl]-2-isopropylbenzenesulfonamide;
5-({4-[(2-cyanoethyl)amino]phenyl}sulfonyl)-N-[2-(1H-imidazol-1-yl)ethyl]-2-isopropylbenzenesulfonamide;
N-[2-(1H-imidazol-1-yl)ethyl]-2-isopropyl-5-{[4-(methylamino)phenyl]sulfonyl}benzenesulfonamide;
5-({4-[(2-hydroxybutyl)amino]phenyl}sulfonyl)-N-[2-(1H-imidazol-1-yl)ethyl]-2-isopropylbenzenesulfonamide;
5-[(4-{[(2S)-1-(hydroxymethyl)-2-methylbutyl]amino}phenyl)sulfonyl]-N-[2-(1H-imidazol-1-yl)ethyl]-2-isopropylbenzenesulfonamide;
N-[3-(1H-imidazol-1-yl)propyl]-2-methyl-3-(phenylsulfonyl)benzenesulfonamide;
N-[3-(1H-Imidazol-1-yl)propyl]-5-(phenylsulfonyl)-2-propylbenzenesulfonamide;
5-[(4-Fluorophenyl)sulfonyl]-N-[3-(1H-imidazol-1-yl)propyl]-2-propylbenzenesulfonamide;
5-[(4-fluorophenyl)sulfonyl]-2-isopropyl-N-[2-(2-methyl-1H-imidazol-1-yl)ethyl]benzenesulfonamide;
N-[2-(2-ethyl-1H-imidazol-1-yl)ethyl]-5-[(4-fluorophenyl)sulfonyl]-2-isopropylbenzenesulfonamide;
5-[(4-fluorophenyl)sulfonyl]-2-isopropyl-N-[2-(2-isopropyl-1H-imidazol-1-yl)ethyl]benzenesulfonamide or
N-[3-(1H-Imidazol-1-yl)propyl]-2,4-diisopropyl-5-(phenylsulfonyl)benzenesulfonamide.

33. The compound of claim 1 which is
5-[(4-fluorophenyl)sulfonyl]-2-methyl-N-[2-(1-methylpyrrolidin-2-yl)ethyl]benzenesulfonamide;
5-[(4-fluorophenyl)sulfonyl]-2-methyl-N-(2-piperidin-1-ylethyl)benzenesulfonamide;
5-[(4-fluorophenyl)sulfonyl]-2-methyl-N-(2-morpholin-4-ylethyl)benzenesulfonamide;
5-[(4-fluorophenyl)sulfonyl]-2-methyl-N-[3-(2-oxopyrrolidin-1-yl)propyl]benzenesulfonamide;
N-[3-(dimethylamino)propyl]-5-[(4-fluorophenyl)sulfonyl]-2-methylbenzenesulfonamide;
5-[(4-fluorophenyl)sulfonyl]-N-(2-methoxyethyl)-2-methylbenzenesulfonamide;
5-[(4-fluorophenyl)sulfonyl]-2-methyl-N-(3-pyrrolidin-1-ylpropyl)benzenesulfonamide;
5-[(4-fluorophenyl)sulfonyl]-2-methyl-N-[3-(4-methylpiperazin-1-yl)propyl]benzenesulfonamide;
2-methyl-N-(3-morpholin-4-ylpropyl)-5-(phenylsulfonyl)benzenesulfonamide;
2-methyl-N-[2-(1-methylpyrrolidin-2-yl)ethyl]-5-(phenylsulfonyl)benzenesulfonamide;
2-methyl-5-(phenylsulfonyl)-N-(2-piperidin-1-ylethyl)benzenesulfonamide;
2-methyl-N-(2-morpholin4-ylethyl)-5-(phenylsulfonyl)benzenesulfonamide;
2-methyl-N-[3-(2-oxopyrrolidin-1-yl)propyl]-5-(phenylsulfonyl)benzenesulfonamide;
2-methyl-5-(phenylsulfonyl)-N-(3-pyrrolidin-1-ylpropyl)benzenesulfonamide;
2-methyl-N-[3-(4-methylpiperazin-1-yl)propyl]-5-(phenylsulfonyl)benzenesulfonamide;
2-ethyl-N-(2-morpholin-4-ylethyl)-5-(phenylsulfonyl)benzenesulfonamide;
2-ethyl-5-(phenylsulfonyl)-N-(3-pyrrolidin-1-ylpropyl)benzenesulfonamide;
2-methoxy-N-(2-morpholin-4-ylethyl)-5-(phenylsulfonyl)benzenesulfonamide;
2-methoxy-5-(phenylsulfonyl)-N-(3-pyrrolidin-1-ylpropyl)benzenesulfonamide;
5-[(4-chlorophenyl)sulfonyl]-2-methyl-N-(2-morpholin4-ylethyl)benzenesulfonamide;
2-methyl-5-(phenylsulfonyl)-N-tetrahydro-2H-pyran-4-ylbenzenesulfonamide;
2-methyl-5-(phenylsulfonyl)-N-(2-tetrahydro-2H-pyran-4-ylethyl)benzenesulfonamide;
2-ethyl-5-(phenylsulfonyl)-N-tetrahydro-2H-pyran4-ylbenzenesulfonamide;
2-ethyl-5-(phenylsulfonyl)-N-(2-tetrahydro-2H-pyran-4-ylethyl)benzenesulfonamide;
5-[(4-fluorophenyl)sulfonyl]-2-methyl-N-tetrahydro-2H-pyran-4-ylbenzenesulfonamide;
5-[(4-fluorophenyl)sulfonyl]-2-methyl-N-(2-tetrahydro-2H-pyran-4-ylethyl)benzenesulfonamide;
5-[(4-fluorophenyl)sulfonyl]-2-isopropyl-N-tetrahydro-2H-pyran-4-ylbenzenesulfonamide;
5-[(4-fluorophenyl)sulfonyl]-2-isopropyl-N-(2-tetrahydro-2H-pyran-4-ylethyl)benzenesulfonamide;
2,4-dimethyl-5-(phenylsulfonyl)-N-tetrahydro-2H-pyran-4-ylbenzenesulfonamide;
2,4-dimethyl-5-(phenylsulfonyl)-N-(2-tetrahydro-2H-pyran-4-ylethyl)benzenesulfonamide;
5-[(4-fluorophenyl)sulfonyl]-2,4-dimethyl-N-(tetrahydro-2H-pyran-4-yl)benzenesulfonamide;
5-[(4-fluorophenyl)sulfonyl]-2,4-dimethyl-N-[2-(tetrahydro-2H-pyran-4-yl)ethyl]benzenesulfonamide;
2-chloro-N-(2-morpholin-4-ylethyl)-5-(phenylsulfonyl)benzenesulfonamide;
2,3-dimethyl-5-(phenylsulfonyl)-N-(tetrahydro-2H-pyran-4-yl)benzenesulfonamide;
2,3-dimethyl-5-(phenylsulfonyl)-N-[2-(tetrahydro-2H-pyran-4-yl)ethyl]benzenesulfonamide;
2-isopropyl-5-{[4-(methylamino)phenyl]sulfonyl}-N-(tetrahydro-2H-pyran-4-yl)benzenesulfonamide;
2-isopropyl-5-{[4-(methylamino)phenyl]sulfonyl}-N-[2-(tetrahydro-2H-pyran-4-yl)ethyl]benzenesulfonamide;
2-chloro-5-(phenylsulfonyl)-N-(tetrahydro-2H-pyran-4-yl)benzenesulfonamide;
5-[(4-bromophenyl)sulfonyl]-2-methyl-N-(tetrahydro-2H-pyran-4-yl)benzenesulfonamide;
5-[(4-cyanophenyl)sulfonyl]-2-methyl-N-(tetrahydro-2H-pyran-4-yl)benzenesulfonamide;
N-[3-(1H-imidazol-1-yl)propyl]-2-methyl-5-(pyridin-2-ylsulfonyl)benzenesulfonamide;
5-[(2,4-dichlorophenyl)sulfonyl]-N-[3-(1H-imidazol-1-yl)propyl]-2-methylbenzenesulfonamide;
5-[(4-acetylphenyl)sulfonyl]-2-methyl-N-(tetrahydro-2H-pyran-4-yl)benzenesulfonamide;
5-[(4-bromophenyl)sulfonyl]-2-methyl-N-(2-pyridin-2-ylethyl)benzenesulfonamide;
2-methyl-5-(phenylsulfonyl)-N-(tetrahydro-2H-pyran-4-ylmethyl)benzenesulfonamide;
5-[(4-fluorophenyl)sulfonyl]-2-methyl-N-(tetrahydro-2H-pyran-4-ylmethyl)benzenesulfonamide;
2-ethyl-5-[(4-fluorophenyl)sulfonyl]-N-(tetrahydro-2H-pyran-4-ylmethyl)benzenesulfonamide;
2,4-dimethyl-5-(phenylsulfonyl)-N-(tetrahydro-2H-pyran-4-ylmethyl)benzenesulfonamide;
N-(2,2-dimethylpropyl)-2-methyl-5-(phenylsulfonyl)benzenesulfonamide;
5-({4-[(1E)-N-hydroxyethanimidoyl]phenyl}sulfonyl)-2-methyl-N-(tetrahydro-2H-pyran-4-yl)benzenesulfonamide;
5-[(4-acetylphenyl)sulfonyl]-2-methyl-N-(2-pyridin-2-ylethyl)benzenesulfonamide;
5-{[4-(1-hydroxy-1-methylethyl)phenyl]sulfonyl}-2-methyl-N-(2-pyridin-2-ylethyl)benzenesulfonamide;
2-methyl-5-(phenylsulfonyl)-N-(2,2,6,6-tetramethylpiperidin-4-yl)benzenesulfonamide;
N-(1-benzylpiperidin-4-yl)-2-methyl-5-(phenylsulfonyl)benzenesulfonamide;
N-(1-benzylpyrrolidin-3-yl)-2-methyl-5-(phenylsulfonyl)benzenesulfonamide;
ethyl 4-({[2-methyl-5-(phenylsulfonyl)phenyl]sulfonyl}amino)piperidine-1-carboxylate;
5-[(4-tert-butylphenyl)sulfonyl]-2-isopropyl-N-(tetrahydro-2H-pyran-4-yl)benzenesulfonamide;
5-[(4-tert-butylphenyl)sulfonyl]-2-methyl-N-(tetrahydro-2H-pyran-4-yl)benzenesulfonamide;
5-[(4-fluorophenyl)sulfonyl]-2-isopropyl-N-(tetrahydro-2H-pyran-4-ylmethyl)benzenesulfonamide;
tert-butyl 4-({[2-methyl-5-(phenylsulfonyl)phenyl]sulfonyl}amino)piperidine-1-carboxylate;
2-methyl-5-(phenylsulfonyl)-N-piperidin-4-ylbenzenesulfonamide;
2-methyl-5-(phenylsulfonyl)-N-[1-(phenylsulfonyl)piperidin-4-yl]benzenesulfonamide;
N-[1-(2-furoyl)piperidin-4-yl]-2-methyl-5-(phenylsulfonyl)benzenesulfonamide;
N-[1-(2-methoxybenzoyl)piperidin-4-yl]-2-methyl-5-(phenylsulfonyl)benzenesulfonamide;
N-[1-(3-methoxybenzoyl)piperidin-4-yl]-2-methyl-5-(phenylsulfonyl)benzenesulfonamide;
N-[1-(3,4-dimethoxybenzoyl)piperidin-4-yl]-2-methyl-5-(phenylsulfonyl)benzenesulfonamide;
2-methyl-5-(phenylsulfonyl)-N-{1-[3-(trifluoromethyl)benzoyl]piperidin-4-yl}benzenesulfonamide;
N-[1-(4-chlorobenzoyl)piperidin-4-yl]-2-methyl-5-(phenylsulfonyl)benzenesulfonamide;
N-[1-(4-methoxybenzoyl)piperidin-4-yl]-2-methyl-5-(phenylsulfonyl)benzenesulfonamide;
2-methyl-N-[1-(4-methylbenzoyl)piperidin-4-yl]-5-(phenylsulfonyl)benzenesulfonamide;
N-[1-(methoxyacetyl)piperidin-4-yl]-2-methyl-5-(phenylsulfonyl)benzenesulfonamide;
2-methyl-N-[1-(phenylacetyl)piperidin-4-yl]-5-(phenylsulfonyl)benzenesulfonamide;
N-[1-(cyclohexylcarbonyl)piperidin-4-yl]-2-methyl-5-(phenylsulfonyl)benzenesulfonamide;
2,6-dimethyl-3-(phenylsulfonyl)-N-(tetrahydro-2H-pyran-4-yl)benzenesulfonamide;
N-[1-(cyclopropylcarbonyl)piperidin-4-yl]-2-methyl-5-(phenylsulfonyl)benzenesulfonamide;
N-[1-(4-cyanobenzoyl)piperidin-4-yl]-2-methyl-5-(phenylsulfonyl)benzenesulfonamide;
N-[1-(3-cyanobenzoyl)piperidin-4-yl]-2-methyl-5-(phenylsulfonyl)benzenesulfonamide;
2-methyl-N-[1-(methylsulfonyl)piperidin-4-yl]-5-(phenylsulfonyl)benzenesulfonamide;
N-(1-acetylpiperidin-4-yl)-2-methyl-5-(phenylsulfonyl)benzenesulfonamide
N-(4-{[4-({[2-methyl-5-(phenylsulfonyl)phenyl]sulfonyl}amino)piperidin-1-yl]carbonyl}phenyl)acetamide;
2-methyl-N-{1-[(1-methyl-1H-imidazol-4-yl)sulfonyl]piperidin-4-yl}-5-(phenylsulfonyl)benzenesulfonamide;
2-methyl-5-(phenylsulfonyl)-N-[1-(2-thienylsulfonyl)piperidin-4-yl]benzenesulfonamide;
N-(1-{[5-(dimethylamino)-1-naphthyl]sulfonyl}piperidin-4-yl)-2-methyl-5-(phenylsulfonyl)benzenesulfonamide;
N-[1-(1,3-benzodioxol-5-ylcarbonyl)piperidin-4-yl]-2-methyl-5-(phenylsulfonyl)benzenesulfonamide;
N-[1-(isoxazol-5-ylcarbonyl)piperidin-4-yl]-2-methyl-5-(phenylsulfonyl)benzenesulfonamide;
N-[1-(N,N-dimethylglycyl)piperidin-4-yl]-2-methyl-5-(phenylsulfonyl)benzenesulfonamide;
prop-2-yn-1-yl 4-({[2-methyl-5-(phenylsulfonyl)phenyl]sulfonyl}amino)piperidine-1carboxylate;
methyl 4-({[2-methyl-5-(phenylsulfonyl)phenyl]sulfonyl}amino)piperidine-1-carboxylate;
2-methoxyphenyl 4-({[2-methyl-5-(phenylsulfonyl)phenyl]sulfonyl}amino)piperidine-1-carboxylate;
N-(tert-butyl)-4-({[2-methyl-5-(phenylsulfonyl)phenyl]sulfonyl}amino)piperidine-1-carboxamide;
N-cyclohexyl-4-({[2-methyl-5-(phenylsulfonyl)phenyl]sulfonyl}amino)piperidine-1-carboxamide;
2-methyl-N-(2-morpholin-4-ylethyl)-3-(phenylsulfonyl)benzenesulfonamide;
2-methyl-N-[1-(2-naphthoyl)piperidin-4-yl]-5-(phenylsulfonyl)benzenesulfonamide;
2-methyl-5-(phenylsulfonyl)-N-[1-(2-thienylcarbonyl)piperidin-4-yl]benzenesulfonamide;
isobutyl 4-({[2-methyl-5-(phenylsulfonyl)phenyl]sulfonyl}amino)piperidine-1-carboxylate;
N-{1-[4-(dimethylamino)benzoyl]piperidin-4-yl}-2-methyl-5-(phenylsulfonyl)benzenesulfonamide;
4-fluorophenyl 4-({[2-methyl-5-(phenylsulfonyl)phenyl]sulfonyl}amino)piperidine-1-carboxylate
N-ethyl-4-({[2-methyl-5-(phenylsulfonyl)phenyl]sulfonyl}amino)piperidine-1-carboxamide;
2-methyl-N-[1-(morpholin-4-ylcarbonyl)piperidin-4-yl]-5-(phenylsulfonyl)benzenesulfonamide;
N,N-dimethyl-4-({[2-methyl-5-(phenylsulfonyl)phenyl]sulfonyl}amino)piperidine-1-carboxamide;
N-[1-(3,3-dimethylbutanoyl)piperidin-4-yl]-2-methyl-5-(phenylsulfonyl)benzenesulfonamide;
2-methyl-5-(phenylsulfonyl)-N-[1-(pyridin-3-ylcarbonyl)piperidin-4-yl]benzenesulfonamide;
tert-butyl 4-[({5-[(4-fluorophenyl)sulfonyl]-2-isopropylphenyl}sulfonyl)amino]piperidine-1-carboxylate;
N-(1-{[5-(dimethylamino)-1-naphthyl]sulfonyl}piperidin-4-yl)-5-[(4-fluorophenyl)sulfonyl]-2-isopropylbenzenesulfonamide;
5-[(4-fluorophenyl)sulfonyl]-2-isopropyl-N-[1-(methoxyacetyl)piperidin-4-yl]benzenesulfonamide;
5-[(4-fluorophenyl)sulfonyl]-2-isopropyl-N-piperidin-4-ylbenzenesulfonamide;
N-(1-benzylpiperidin-4-yl)-5-[(4-fluorophenyl)sulfonyl]-2-isopropylbenzenesulfonamide;
ethyl 4-[({5-[(4-fluorophenyl)sulfonyl]-2-isopropylphenyl}sulfonyl)amino]piperidine-1-carboxylate;
5-[(4-fluorophenyl)sulfonyl]-2-isopropyl-N-(2-piperidin-1-ylethyl)benzenesulfonamide;
tert-butyl 4-{[(2-isopropyl-5-{[4-(methylamino)phenyl]sulfonyl}phenyl)sulfonyl]amino}piperidine-1-carboxylate;
N-(1-acetylpiperidin-4-yl)-5-[(4-fluorophenyl)sulfonyl]-2-isopropylbenzenesulfonamide;
N-[1-(cyclopropylcarbonyl)piperidin-4-yl]-5-[(4-fluorophenyl)sulfonyl]-2-isopropylbenzenesulfonamide;
N-[1-(4-cyanobenzoyl)piperidin-4-yl]-5-[(4-fluorophenyl)sulfonyl]-2-isopropylbenzenesulfonamide;
5-[(4-fluorophenyl)sulfonyl]-2-isopropyl-N-(tetrahydrofuran-2-ylmethyl)benzenesulfonamide;
5-({4-[(2-cyanoethyl)amino]phenyl}sulfonyl)-2-isopropyl-N-(tetrahydro-2H-pyran-4-yl)benzenesulfonamide;
5-({4-[(2-cyanoethyl)amino]phenyl}sulfonyl)-2-isopropyl-N-(tetrahydro-2H-pyran-4-ylmethyl)benzenesulfonamide;
5-({4-[(2-cyanoethyl)amino]phenyl}sulfonyl)-2-isopropyl-N-[2-(tetrahydro-2H-pyran-4-yl)ethyl]benzenesulfonamide;
N-(3′,6′-dihydroxy-3-oxo-3H-spiro[2-benzofuran-1,9′-xanthen]-5-yl)-4-[({5-[(4-fluorophenyl)sulfonyl]-2-isopropylphenyl}sulfonyl)amino]piperidine-1-carbothioamide;
b 2-isopropyl-5-[(2-methylphenyl)sulfonyl]-N-(tetrahydro-2H-pyran-4-yl)benzenesulfonamide;
5-[(3-chloro-2-methylphenyl)sulfonyl]-2-isopropyl-N-(tetrahydro-2H-pyran-4-yl)benzenesulfonamide;
2-isopropyl-5-{[4-(methylamino)phenyl]sulfonyl}-N-(tetrahydro-2H-pyran-4-ylmethyl)benzenesulfonamide;
5-{[4-(dimethylamino)phenyl]sulfonyl}-2-isopropyl-N-(tetrahydro-2H-pyran-4-yl)benzenesulfonamide;
5-{[4-(dimethylamino)phenyl]sulfonyl}-2-isopropyl-N-(tetrahydro-2H-pyran-4-ylmethyl)benzenesulfonamide;
5-{[4-(dimethylamino)phenyl]sulfonyl}-2-isopropyl-N-[2-(tetrahydro-2H-pyran-4-yl)ethyl]benzenesulfonamide;
2-isopropyl-5-{[4-(4-methylpiperazin-1-yl)phenyl]sulfonyl}-N-(tetrahydro-2H-pyran-4-yl)benzenesulfonamide;
2-isopropyl-5-{[4-(4-methylpiperazin-1-yl)phenyl]sulfonyl}-N-(tetrahydro-2H-pyran-4-ylmethyl)benzenesulfonamide;
2-isopropyl-5-{[4-(4-methylpiperazin-1-yl)phenyl]sulfonyl}-N-[2-(tetrahydro-2H-pyran-4-yl)ethyl]benzenesulfonamide;
5-(phenylsulfonyl)-2-propyl-N-(tetrahydro-2H-pyran-4-yl)benzenesulfonamide;
5-(phenylsulfonyl)-2-propyl-N-(tetrahydro-2H-pyran-4-ylmethyl)benzenesulfonamide;
5-(phenylsulfonyl)-2-propyl-N-[2-(tetrahydro-2H-pyran-4-yl)ethyl]benzenesulfonamide;
tert-butyl 4-({[5-(phenylsulfonyl)-2-propylphenyl]sulfonyl}amino)piperidine-1-carboxylate;
5-[(4-fluorophenyl)sulfonyl]-2-propyl-N-(tetrahydro-2H-pyran-4-yl)benzenesulfonamide;
5-[(4-fluorophenyl)sulfonyl]-2-propyl-N-(tetrahydro-2H-pyran-4-ylmethyl)benzenesulfonamide;
5-[(4-fluorophenyl)sulfonyl]-2-propyl-N-[2-(tetrahydro-2H-pyran-4-yl)ethyl]benzenesulfonamide;
tert-Butyl 4-[({5-[(4-fluorophenyl)sulfonyl]-2-propylphenyl}sulfonyl)amino]piperidine-1-carboxylate;
2-isopropyl-5-{[4-(methylamino)phenyl]sulfonyl}-N-piperidin-4-ylbenzenesulfonamide;
5-[(5-chloro-1,3-dimethyl-1H-pyrazol-4-yl)sulfonyl]-2-isopropyl-N-(tetrahydro-2H-pyran-4-yl)benzenesulfonamide;
5-[(4-fluorophenyl)sulfonyl]-2-isopropyl-N-[1-(morpholin-4-ylcarbonyl)piperidin-4-yl]benzenesulfonamide;
4-[({5-[(4-fluorophenyl)sulfonyl]-2-isopropylphenyl}sulfonyl)amino]-N,N-dimethylpiperidine-1-carboxamide;
N-(1-benzylpiperidin-4-yl)-5-[(4-fluorophenyl)sulfonyl]-2-methylbenzenesulfonamide;
5-[(4-fluorophenyl)sulfonyl]-2-methyl-N-piperidin-4-ylbenzenesulfonamide;
5-({4-[(2-cyanoethyl)(methyl)amino]phenyl}sulfonyl)-2-isopropyl-N-(tetrahydro-2H-pyran-4-yl)benzenesulfonamide;
5-({4-[(2-cyanoethyl)(methyl)amino]phenyl}sulfonyl)-2-isopropyl-N-(tetrahydro-2H-pyran-4-ylmethyl)benzenesulfonamide;
N-(1-acetylpiperidin-4-yl)-5-[(4-fluorophenyl)sulfonyl]-2-methylbenzenesulfonamide;
N-[1-(4-cyanobenzoyl)piperidin-4-yl]-5-[(4-fluorophenyl)sulfonyl]-2-methylbenzenesulfonamide;
4-[({5-[(4-fluorophenyl)sulfonyl]-2-methylphenyl}sulfonyl)amino]-N,N-dimethylpiperidine-1-carboxamide;
5-[(4-fluorophenyl)sulfonyl]-N-[1-(methoxyacetyl)piperidin-4-yl]-2-methylbenzenesulfonamide;
2-isopropyl-5-[(3-methoxyphenyl)sulfonyl]-N-(tetrahydro-2H-pyran-4-yl)benzenesulfonamide;
5-{[4-(dimethylamino)-2-methylphenyl]sulfonyl}-2-isopropyl-N-(2-pyridin-2-ylethyl)benzenesulfonamide;
2-(dimethylamino)-5-(phenylsulfonyl)-N-(2-pyridin-2-ylethyl)benzenesulfonamide;
5-[(4-fluorophenyl)sulfonyl]-2-methyl-N-[1-(morpholin-4-ylcarbonyl)piperidin-4-yl]benzenesulfonamide;
2-chloro-5-[(3-methylphenyl)sulfonyl]-N-(tetrahydro-2H-pyran-4-yl)benzenesulfonamide;
2-chloro-5-[(3-methoxyphenyl)sulfonyl]-N-(tetrahydro-2H-pyran-4-yl)benzenesulfonamide;
2-chloro-5-[(1-methyl-1H-indol-5-yl)sulfonyl]-N-(tetrahydro-2H-pyran-4-yl)benzenesulfonamide;
5-(phenylsulfonyl)-N-(tetrahydro-2H-pyran-4-yl)-2-(trifluoromethyl)benzenesulfonamide;
2,4-diisopropyl-5-(phenylsulfonyl)-N-(tetrahydro-2H-pyran-4-yl)benzenesulfonamide;
2,4-diisopropyl-5-(phenylsulfonyl)-N-(tetrahydro-2H-pyran-4-ylmethyl)benzenesulfonamide;
2,4-diisopropyl-5-(phenylsulfonyl)-N-[2-(tetrahydro-2H-pyran-4-yl)ethyl]benzenesulfonamide;
5-[(4-Fluorophenyl)sulfonyl]-2,4-diisopropyl-N-(tetrahydro-2H-pyran-4-yl)benzenesulfonamide;
2-chloro-5-[(4-fluorophenyl)sulfonyl]-N-(tetrahydro-2H-pyran-4-yl)benzenesulfonamide;
tert-butyl 4-({[2-chloro-5-(phenylsulfonyl)phenyl]sulfonyl}amino)piperidine-1-carboxylate
2-chloro-5-(phenylsulfonyl)-N-piperidin-4-ylbenzenesulfonamide;
tert-butyl 4-[4-({4-isopropyl-3-[(tetrahydro-2H-pyran-4-ylamino)sulfonyl]phenyl}sulfonyl)phenyl]piperazine-1-carboxylate
5-({4-cis-3,5-dimethylpiperazin-1-ylphenyl}sulfonyl)-2-isopropyl-N-(tetrahydro-2H-pyran-4yl)benzenesulfonamide;
5-([4-trans-2,5-dimethylpiperazin-1-ylphenyl}sulfonyl)-2-isopropyl-N-(tetrahydro-2H-pyran-4yl)benzenesulfonamide;
2-isopropyl-5-[(4-piperazin-1-ylphenyl)sulfonyl]-N-(tetrahydro-2H-pyran-4-yl)benzenesulfonamide;
5-[(4-Fluorophenyl)sulfonyl]-2,4-diisopropyl-N-(tetrahydro-2H-pyran-4-ylmethyl)benzenesulfonamide;
5-[(4-Fluorophenyl)sulfonyl]-2,4-diisopropyl-N-[2-(tetrahydro-2H-pyran-4-yl)ethyl]benzenesulfonamide;
2-chloro-N-[3-(4-methylpiperazin-1-yl)propyl]-5-(phenylsulfonyl)benzenesulfonamide;
1-{[2-chloro-5-(phenylsulfonyl)phenyl]sulfonyl}-N,N-diethylpyrrolidin-3-amine;
ethyl 1-{[2-chloro-5-(phenylsulfonyl)phenyl]sulfonyl}piperidine-3-carboxylate;
2-chloro-5-(phenylsulfonyl)-N-[1-(trifluoroacetyl)piperidin-4-yl]benzenesulfonamide;
2-chloro-N-[1-(2,2-dimethylpropanoyl)piperidin-4-yl]-5-(phenylsulfonyl)benzenesulfonamide;
N-(tert-butyl)-4-({[2-chloro-5-(phenylsulfonyl)phenyl]sulfonyl}amino)piperidine-1-carboxamide;
2-chloro-N-[1-(morpholin-4-ylcarbonyl)piperidin-4-yl]-5-(phenylsulfonyl)benzenesulfonamide; or
2-chloro-N-(1-cyanopiperidin-4-yl)-5-(phenylsulfonyl)benzenesulfonamide.

34. The compound of claim 1 which is
N³-({5-[(4-fluorophenyl)sulfonyl]-2-methylphenyl}sulfonyl)-beta-alaninamide;
methyl N-({5-[(4-fluorophenyl)sulfonyl]-2-methylphenyl}sulfonyl)-beta-alaninate;
N-(2-cyanoethyl)-5-[(4-fluorophenyl)sulfonyl]-2-methylbenzenesulfonamide;
N-{2-[({5-[(4-fluorophenyl)sulfonyl]-2-methylphenyl}sulfonyl)amino]ethyl}acetamide;
N-[2-(diethylamino)ethyl]-5-[(4-fluorophenyl)sulfonyl]-2-methylbenzenesulfonamide;
N-[2-(dimethylamino)ethyl]-5-[(4-fluorophenyl)sulfonyl]-2-methylbenzenesulfonamide;
5-[(4-fluorophenyl)sulfonyl]-N-(3-methoxypropyl)-2-methylbenzenesulfonamide;
N-[3-(diethylamino)propyl]-5-[(4-fluorophenyl)sulfonyl]-2-methylbenzenesulfonamide;
N-({5-[(4-fluorophenyl)sulfonyl]-2-methylphenyl}sulfonyl)-beta-alanine;
N³-{[2-methyl-5-(phenylsulfonyl)phenyl]sulfonyl}-beta-alaninamide;
methyl N-{[2-methyl-5-(phenylsulfonyl)phenyl]sulfonyl}-beta-alaninate;
N-[2-(diethylamino)ethyl]-2-methyl-5-(phenylsulfonyl)benzenesulfonamide;
N-[2-(dimethylamino)ethyl]-2-methyl-5-(phenylsulfonyl)benzenesulfonamide;
N-(3-methoxypropyl)-2-methyl-5-(phenylsulfonyl)benzenesulfonamide;
N-[3-(dimethylamino)propyl]-2-methyl-5-(phenylsulfonyl)benzenesulfonamide;
N-(2-methoxyethyl)-2-methyl-5-(phenylsulfonyl)benzenesulfonamide;
N-[3-(diethylamino)propyl]-2-methyl-5-(phenylsulfonyl)benzenesulfonamide;
methyl N-{[2-ethyl-5-(phenylsulfonyl)phenyl]sulfonyl}-beta-alaninate;
2-ethyl-N-(3-methoxypropyl)-5-(phenylsulfonyl)benzenesulfonamide;
N-[3-(dimethylamino)propyl]-2-ethyl-5-(phenylsulfonyl)benzenesulfonamide;
2-ethyl-N-(2-methoxyethyl)-5-(phenylsulfonyl)benzenesulfonamide;
N-[3-(diethylamino)propyl]-2-ethyl-5-(phenylsulfonyl)benzene sulfonamide;
methyl N-{[2-methoxy-5-(phenylsulfonyl)phenyl]sulfonyl}-beta-alaninate;
2-methoxy-N-(3-methoxypropyl)-5-(phenylsulfonyl)benzenesulfonamide;
N-[3-(dimethylamino)propyl]-2-methoxy-5-(phenylsulfonyl)benzenesulfonamide;
2-methoxy-N-(2-methoxyethyl)-5-(phenylsulfonyl)benzenesulfonamide;
N-[3-(diethylamino)propyl]-2-methoxy-5-(phenylsulfonyl)benzenesulfonamide;
2-methyl-5-(phenylsulfonyl)-N-propylbenzenesulfonamide;
N-(1-ethylpropyl)-2-methyl-5-(phenylsulfonyl)benzenesulfonamide;
N-cyclobutyl-2-methyl-5-(phenylsulfonyl)benzenesulfonamide;
N-cyclopentyl-2-methyl-5-(phenylsulfonyl)benzenesulfonamide;
N-cyclohexyl-2-methyl-5-(phenylsulfonyl)benzenesulfonamide;
2-methyl-5-(phenylsulfonyl)-N-(2,2,2-trifluoroethyl)benzenesulfonamide;
N-(2-hydroxy-1,1-dimethylethyl)-2-methyl-5-(phenylsulfonyl)benzenesulfonamide;
N-cyclopropyl-2-methyl-5-(phenylsulfonyl)benzenesulfonamide;
N-cyclopropyl-5-[(4-fluorophenyl)sulfonyl]-2-isopropylbenzenesulfonamide;
N-cyclobutyl-5-[(4-fluorophenyl)sulfonyl]-2-isopropylbenzenesulfonamide;
N-cyclopentyl-5-[(4-fluorophenyl)sulfonyl]-2-isopropylbenzenesulfonamide;
N-cyclohexyl-5-[(4-fluorophenyl)sulfonyl]-2-isopropylbenzenesulfonamide;
N-cyclopentyl-5-(phenylsulfonyl)-2-propylbenzenesulfonamide;
N-cyclopentyl-5-[(4-fluorophenyl)sulfonyl]-2-propylbenzenesulfonamide;
N-cyclopentyl-2,4-diisopropyl-5-(phenylsulfonyl)benzenesulfonamide;
N-cyclopentyl-5-[(4-fluorophenyl)sulfonyl]-2,4-diisopropylbenzenesulfonamide;
2-chloro-N-(2-cyanoethyl)-5-(phenylsulfonyl)benzenesulfonamide; or
methyl N-{[2-chloro-5-(phenylsulfonyl)phenyl]sulfonyl}-2-methylalaninate.

35. The compound of claim 1 which is
N-[2-(3,4-dimethoxyphenyl)ethyl]-N,2-dimethyl-5-(phenylsulfonyl)benzenesulfonamide;
N-allyl-N-[2-(3,4-dimethoxyphenyl)ethyl]-2-methyl-5-(phenylsulfonyl)benzenesulfonamide;
N-[2-(3,4-dimethoxyphenyl)ethyl]-2-methyl-5-(phenylsulfonyl)-N-prop-2-ynylbenzenesulfonamide;
N-[2-(2-fluorophenyl)ethyl]-N,2-dimethyl-5-[(4-methylphenyl)sulfonyl]benzenesulfonamide;
N-allyl-N-[2-(2-fluorophenyl)ethyl]-2-methyl-5-[(4-methylphenyl)sulfonyl]benzene sulfonamide;
N-[2-(2-fluorophenyl)ethyl]-2-methyl-5-[(4-methylphenyl)sulfonyl]-N-prop-2-ynylbenzenesulfonamide;
N,2-dimethyl-N-(2-phenylethyl)-5-(phenylsulfonyl)benzenesulfonamide;
1-{[2-methyl-5-(phenylsulfonyl)phenyl]sulfonyl}-4-(2-oxo-2-pyrrolidin-1-ylethyl)piperazine;
N,N-diethyl-N-[2-(4-{[2-methyl-5-(phenylsulfonyl)phenyl]sulfonyl}piperazin-1-yl)ethyl]amine;
4-[2-(4-{[²-methyl-5-(phenylsulfonyl)phenyl]sulfonyl}piperazin-1-yl)ethyl]morpholine;
1-{[2-methyl-5-(phenylsulfonyl)phenyl]sulfonyl}4-(2-pyrrolidin-1-ylethyl)piperazine;
4-[3-(4-{[2-methyl-5-(phenylsulfonyl)phenyl]sulfonyl}piperazin-1-yl)propyl]morpholine;
2-ethyl-N-methyl-N-(2-phenylethyl)-5-(phenylsulfonyl)benzenesulfonamide;
1-{[2-ethyl-5-(phenylsulfonyl)phenyl]sulfonyl}-4-(2-oxo-2-pyrrolidin-1-ylethyl)piperazine;
N,N-diethyl-N-[2-(4-{[2-ethyl-5-(phenylsulfonyl)phenyl]sulfonyl}piperazin-1-yl)ethyl]amine;
4-[2-(4-{[2-ethyl-5-(phenylsulfonyl)phenyl]sulfonyl}piperazin-1-yl)ethyl]morpholine;
1-{[2-ethyl-5-(phenylsulfonyl)phenyl]sulfonyl}-4-(2-pyrrolidin-1-ylethyl)piperazine;
4-[3-(4-{[2-ethyl-5-(phenylsulfonyl)phenyl]sulfonyl}piperazin-1-yl)propyl]morpholine;
N-[1-(cyclopropylcarbonyl)piperidin-4-yl]-N-({5-[(4-fluorophenyl)sulfonyl]-2-methylphenyl}sulfonyl)cyclopropanecarboxamide; p1 1-{[2-chloro-5-(phenylsulfonyl)phenyl]sulfonyl}-4-pyrrolidin-1-ylpiperidine;
4-[2-(4-{[2-chloro-5-(phenylsulfonyl)phenyl]sulfonyl}piperazin-1-yl)ethyl]morpholine;
1-(1,3-benzodioxol-5-ylmethyl)-4-{[2-chloro-5-(phenylsulfonyl)phenyl]sulfonyl}piperazine;
tert-butyl (1-{[2-chloro-5-(phenylsulfonyl)phenyl]sulfonyl}pyrrolidin-3-yl)carbamate;
tert-butyl (1-{[2-chloro-5-(phenylsulfonyl)phenyl]sulfonyl}piperidin-4-yl)carbamate;
2-chloro-N-methyl-5-(phenylsulfonyl)-N-(2-pyridin-2-ylethyl)benzenesulfonamide; or
1-{[2-chloro-5-(phenylsulfonyl)phenyl]sulfonyl}-4-[(2,5-dimethyl-1H-pyrrol-1-yl)methyl]piperidine.

36. The compound of claim 1 which is
2-chloro-N-(2-hydroxy-1,1-dimethylethyl)-5-(phenylsulfonyl)benzenesulfonamide;
2-chloro-N-(cyanomethyl)-5-(phenylsulfonyl)benzenesulfonamide;
N-(2-cyanoethyl)-5-[(4-fluorophenyl)sulfonyl]-2-isopropylbenzenesulfonamide;
2-methyl-N-(3-oxo-3-pyrrolidin-1-ylpropyl)-5-(phenylsulfonyl)benzenesulfonamide;
N-(tert-butyl)-N³-{[2-methyl-5-(phenylsulfonyl)phenyl]sulfonyl}-β-alaninamide;
N³-{[2-methyl-5-(phenylsulfonyl)phenyl]sulfonyl}-N-(1,2,3,4-tetrahydronaphthalen-1-yl)-β-alaninamide;
N-methyl-N³-{[2-methyl-5-(phenylsulfonyl)phenyl]sulfonyl}-N-phenyl-β-alaninamide;
2-methyl-N-[3-(6-methyl-3,4-dihydroquinolin-1(2H)-yl)-3-oxopropyl]-5-(phenylsulfonyl)benzenesulfonamide;
2-chloro-N-(2-hydroxyethyl)-5-(phenylsulfonyl)benzenesulfonamide;
2-chloro-N-(2-hydroxy-1-methylethyl)-5-(phenylsulfonyl)benzenesulfonamide;
2-chloro-N-[2-hydroxy-1-(hydroxymethyl)ethyl]-5-(phenylsulfonyl)benzenesulfonamide;
N-(2-cyanoethyl)-5-(phenylsulfonyl)-2-(trifluoromethyl)benzenesulfonamide;
N-(2-hydroxyethyl)-5-(phenylsulfonyl)-2-(trifluoromethyl)benzenesulfonamide;
N-(2-hydroxy-1-methylethyl)-5-(phenylsulfonyl)-2-(trifluoromethyl)benzenesulfonamide;
N-[(1S)-2-hydroxy-1-methylethyl]-5-(phenylsulfonyl)-2-(trifluoromethyl)benzenesulfonamide;
N-[(1R)-2-hydroxy-1-methylethyl]-5-(phenylsulfonyl)-2-(trifluoromethyl)benzenesulfonamide;
5-[(4-fluorophenyl)sulfonyl]-N-(2-hydroxy-1-methylethyl)-2-isopropylbenzenesulfonamide;
5-[(4-fluorophenyl)sulfonyl]-N-[1-(hydroxymethyl)-2-methylpropyl]-2-isopropylbenzenesulfonamide;
5-[(4-fluorophenyl)sulfonyl]-N-(2-hydroxybutyl)-2-isopropylbenzenesulfonamide;
N-(2-cyanoethyl)-5-[(3-methoxyphenyl)sulfonyl]-2-(trifluoromethyl)benzenesulfonamide;
5-[(3-chlorophenyl)sulfonyl]-N-(2-cyanoethyl)-2-(trifluoromethyl)benzenesulfonamide;
5-(phenylsulfonyl)-N-(tetrahydro-2H-pyran-4-ylmethyl)-2-(trifluoromethyl)benzenesulfonamide;
N-(2-morpholin-4-ylethyl)-5-(phenylsulfonyl)-2-(trifluoromethyl)benzenesulfonamide;
N-(3-morpholin-4-ylpropyl)-5-(phenylsulfonyl)-2-(trifluoromethyl)benzenesulfonamide;
N-(3-methoxypropyl)-5-(phenylsulfonyl)-2-(trifluoromethyl)benzenesulfonamide;
N-[1-(hydroxymethyl)-2-methylpropyl]-5-(phenylsulfonyl)-2-(trifluoromethyl)benzenesulfonamide;
N-[(1R)-1-(hydroxymethyl)propyl]-5-(phenylsulfonyl)-2-(trifluoromethyl)benzenesulfonamide;
N-(2-hydroxyethyl)-5-[(3-methoxyphenyl)sulfonyl]-2-(trifluoromethyl)benzenesulfonamide;
N-(2-cyanoethyl)-5-[(4-methoxyphenyl)sulfonyl]-2-(trifluoromethyl)benzenesulfonamide;
N-(2-cyanoethyl)-5-[(4-hydroxyphenyl)sulfonyl]-2-(trifluoromethyl)benzenesulfonamide;
N-[(1-hydroxycyclohexyl)methyl]-5-(phenylsulfonyl)-2-(trifluoromethyl)benzenesulfonamide;
5-[(4-fluorophenyl)sulfonyl]-2-isopropyl-N-(2-piperidin-3-ylethyl)benzenesulfonamide;
N-{[(5-(phenylsulfonyl)-2-(trifluoromethyl)phenyl]sulfonyl)}-β-alanine;
4-({[5-(phenylsulfonyl)-2-(trifluoromethyl)phenyl]sulfonyl}amino)butanoic acid;
tert-butyl 4-[({[5-(phenylsulfonyl)-2-(trifluoromethyl)phenyl]sulfonyl}amino)methyl]piperidine-1-carboxylate;
5-(phenylsulfonyl)-N-(piperidin-4-ylmethyl)-2-(trifluoromethyl)benzenesulfonamide;
tert-butyl [trans-4-({[5-(phenylsulfonyl)-2-(trifluoromethyl)phenyl]sulfonyl}amino)cyclohexyl]carbamate;
4-oxo-4-{4-[({[5-(phenylsulfonyl)-2-(trifluoromethyl)phenyl]sulfonyl}amino)methyl]piperidin-1-yl}butanoic acid;
5-oxo-5-4-[({[5-(phenylsulfonyl)-2-(trifluoromethyl)phenyl]sulfonyl}amino)methyl]piperidin-1-yl}pentanoic acid;
3-({4-[({[5-(phenylsulfonyl)-2-(trifluoromethyl)phenyl]sulfonyl}amino)methyl]piperidin-1yl}sulfonyl)benzoic acid;
tert-butyl 2-[2-({[5-(phenylsulfonyl)-2-(trifluoromethyl)phenyl]sulfonyl}amino)ethyl]pyrrolidine-1-carboxylate;
5-(phenylsulfonyl)-N-(2-pyrrolidin-2-ylethyl)-2-(trifluoromethyl)benzenesulfonamide;
{2-[2-({[5-(phenylsulfonyl)-2-(trifluoromethyl)phenyl]sulfonyl)}amino)ethyl]pyrrolidin-1yl}acetic acid;
4-oxo-4-{2-[2-({[5-(phenylsulfonyl)-2-(trifluoromethyl)phenyl]sulfonyl}amino)ethyl]pyrrolidin-1-yl}butanoic acid;
3-({2-[2-({[5-(phenylsulfonyl)-2-(trifluoromethyl)phenyl]sulfonyl}amino)ethyl]pyrrolidin-1-yl}sulfonyl)benzoic acid;
tert-butyl 4-[2-({[5-(phenylsulfonyl)-2-(trifluoromethyl)phenyl]sulfonyl}amino)ethyl]piperidine-1-carboxylate;
methyl 3-({[2-isopropyl-5-(phenylsulfonyl)phenyl]sulfonyl}amino)cyclohexanecarboxylate;
methyl 4-({[2-isopropyl-5-(phenylsulfonyl)phenyl]sulfonyl}amino)cyclohexanecarboxylate;
methyl trans-4-[({[2-isopropyl-5-(phenylsulfonyl)phenyl]sulfonyl}amino)methyl]cyclohexanecarboxylate;
5-(phenylsulfonyl)-N-(2-piperidin-4-ylethyl)-2-(trifluoromethyl)benzenesulfonamide;
{4-[({[5-(phenylsulfonyl)-2-(trifluoromethyl)phenyl]sulfonyl}amino)methyl]piperidin-1-yl}acetic acid;
{4-[2-({[5-(phenylsulfonyl)-2-(trifluoromethyl)phenyl]sulfonyl}amino)ethyl]piperidin-1yl}acetic acid;
methyl 3-({[5-(phenylsulfonyl)-2-(trifluoromethyl)phenyl]sulfonyl}amino)cyclohexanecarboxylate;
methyl 4-({[5-(phenylsulfonyl)-2-(trifluoromethyl)phenyl]sulfonyl}amino)cyclohexanecarboxylate;
methyl trans-4-[({[5-(phenylsulfonyl)-2-(trifluoromethyl)phenyl]sulfonyl}amino)methyl]cyclohexanecarboxylate;
3-({[2-isopropyl-5-(phenylsulfonyl)phenyl]sulfonyl}amino)cyclohexanecarboxylic acid;
4-({[2-isopropyl-5-(phenylsulfonyl)phenyl]sulfonyl}amino)cyclohexanecarboxylic acid;
trans-4-[({[2-isopropyl-5-(phenylsulfonyl)phenyl]sulfonyl}amino)methyl]cyclohexanecarboxylic acid;
3-{4-[({[5-(phenylsulfonyl)-2-(trifluoromethyl)phenyl]sulfonyl}amino)methyl]piperidin-1yl}benzoic acid;
3-{4-[2-({[5-(phenylsulfonyl)-2-(trifluoromethyl)phenyl]sulfonyl}amino)ethyl]piperidin-1yl}benzoic acid;
N-(trans-4-aminocyclohexyl)-5-(phenylsulfonyl)-2-(trifluoromethyl)benzenesulfonamide;
3-({[5-(phenylsulfonyl)-2-(trifluoromethyl)phenyl]sulfonyl}amino)cyclohexanecarboxylic acid;
4-({[5-(phenylsulfonyl)-2-(trifluoromethyl)phenyl]sulfonyl}amino)cyclohexanecarboxylic acid;
trans-4-[({[5-(phenylsulfonyl)-2-(trifluoromethyl)phenyl]sulfonyl}amino)methyl]cyclohexanecarboxylic acid;
N-(trans-4-hydroxycyclohexyl)-5-(phenylsulfonyl)-2-(trifluoromethyl)benzenesulfonamide;
tert-butyl 4-[({[5-(phenylsulfonyl)-2-(trifluoromethyl)phenyl]sulfonyl}amino)carbonyl]piperidine-1-carboxylate; or
N-{[5-(phenylsulfonyl)-2-(trifluoromethyl)phenyl]sulfonyl}piperidine-4-carboxamide.

37. The compound of claim 1 which is
5-(phenylsulfonyl)-N-(tetrahydro-2H-pyran-4-yl)-2-(trifluoromethoxy)benzenesulfonamide;
2-isopropyl-5-(phenylsulfonyl)-N-(tetrahydro-2H-pyran-4-yl)benzenesulfonamide;
5-(phenylsulfonyl)-N-piperidin-4-yl-2-(trifluoromethyl)benzenesulfonamide;
2-Methoxy-5-(phenylsulfonyl)-N-(tetrahydro-2H-pyran-4-yl)benzenesulfonamide;
5-[(3-methoxyphenyl)sulfonyl]-N-(tetrahydro-2H-pyran-4-yl)-2-(trifluoromethyl)benzenesulfonamide;
5-[(3-hydroxyphenyl)sulfonyl]-N-(tetrahydro-2H-pyran-4-yl)-2-(trifluoromethyl)benzenesulfonamide;
5-[(3-chlorophenyl)sulfonyl]-N-(tetrahydro-2H-pyran-4-yl)-2-(trifluoromethyl)benzenesulfonamide;
5-(phenylsulfonyl)-N-[(3S)-piperidin-3-yl]-2-(trifluoromethyl)benzenesulfonamide;
5-(phenylsulfonyl)-N-[(3R)-piperidin-3-yl]-2-(trifluoromethyl)benzenesulfonamide;
5-[(1,2-dimethyl-1H-indol-5-yl)sulfonyl]-N-piperidin-4-yl-2-(trifluoromethyl)benzenesulfonamide;
5-(phenylsulfonyl)-N-[(3S)-pyrrolidin-3-yl]-2-(trifluoromethyl)benzenesulfonamide;
5-(phenylsulfonyl)-N-[(3R)-pyrrolidin-3-yl]-2-(trifluoromethyl)benzenesulfonamide;
2-methyl-5-(phenylsulfonyl)-N-(tetrahydro-2H-thiopyran-4-yl)benzenesulfonamide;
5-[(4-fluorophenyl)sulfonyl]-2-isopropyl-N-(tetrahydro-2H-thiopyran-4-yl)benzenesulfonamide;
5-[(4-fluorophenyl)sulfonyl]-N-(tetrahydro-2H-pyran-4-yl)-2-(trifluoromethyl)benzenesulfonamide;
5-[(2-fluorophenyl)sulfonyl]-N-(tetrahydro-2H-pyran-4-yl)-2-(trifluoromethyl)benzenesulfonamide;
5-(phenylsulfonyl)-N-pyrrolidin-3-yl-2-(trifluoromethyl)benzenesulfonamide;
N-[(2R*,4S*,6S*)-2,6-dimethyltetrahydro-2H-pyran-4-yl]-5-[(4-fluorophenyl)sulfonyl]-2-methylbenzenesulfonamide;
5-{[4-(methylamino)phenyl]sulfonyl}-N-piperidin-4-yl-2-(trifluoromethyl)benzenesulfonamide;
5-{[4-(dimethylamino)phenyl]sulfonyl}-N-piperidin-4-yl-2-(trifluoromethyl)benzenesulfonamide;
5-({4-[(2-hydroxyethyl)amino]phenyl}sulfonyl)-N-piperidin-4-yl-2-(trifluoromethyl)benzenesulfonamide;
N-(1,1-dioxidotetrahydro-2H-thiopyran-4-yl)-5-[(4-fluorophenyl)sulfonyl]-2-isopropylbenzenesulfonamide;
5-{[2-(methylamino)phenyl]sulfonyl}-N-piperidin-4-yl-2-(trifluoromethyl)benzenesulfonamide;
5-{[2-(dimethylamino)phenyl]sulfonyl}-N-piperidin-4-yl-2-(trifluoromethyl)benzenesulfonamide;
5-({2-[(2-hydroxyethyl)amino]phenyl}sulfonyl)-N-piperidin-4-yl-2-(trifluoromethyl)benzenesulfonamide;
2-ethyl-5-(phenylsulfonyl)-N-piperidin-4-ylbenzenesulfonamide;
2,3-dimethyl-5-(phenylsulfonyl)-N-piperidin-4-ylbenzenesulfonamide;
2,4-dimethyl-5-(phenylsulfonyl)-N-piperidin-4-ylbenzenesulfonamide;
2-isopropyl-5-(phenylsulfonyl)-N-piperidin-4-ylbenzenesulfonamide;
5-(phenylsulfonyl)-N-piperidin-4-yl-2-propylbenzenesulfonamide;
2-isopropyl-5-(phenylsulfonyl)-N-(2,2,6,6-tetramethylpiperidin-4-yl)benzenesulfonamide;
5-(phenylsulfonyl)-N-(2,2,6,6-tetramethylpiperidin-4-yl)-2-(trifluoromethyl)benzenesulfonamide;
5-[(4-methoxyphenyl)sulfonyl]-N-piperidin-4-yl-2-(trifluoromethyl)benzenesulfonamide; or
5-[(3-bromophenyl)sulfonyl]-N-piperidin-4-yl-2-(trifluoromethyl)benzenesulfonamide.

38. The compound of claim 1 which is
2-chloro-N-[1-(4-fluorobenzoyl)piperidin-4-yl]-5-(phenylsulfonyl)benzenesulfonamide;
2-chloro-N-[1-(4-cyanobenzoyl)piperidin-4-yl]-5-(phenylsulfonyl)benzenesulfonamide;
2-chloro-5-(phenylsulfonyl)-N-{1-[4-(trifluoromethyl)benzoyl]piperidin-4-yl}benzenesulfonamide;
5-(phenylsulfonyl)-2-(trifluoromethyl)-N-{1-[4-(trifluoromethyl)benzoyl]piperidin-4-yl}benzenesulfonamide;
N-[1-(2-chlorobenzoyl)piperidin-4-yl]-5-(phenylsulfonyl)-2-(trifluoromethyl)benzenesulfonamide;
N-[1-(2-methoxybenzoyl)piperidin-4-yl]-5-(phenylsulfonyl)-2-(trifluoromethyl)benzenesulfonamide;
N-[1-(3-chlorobenzoyl)piperidin-4-yl]-5-(phenylsulfonyl)-2-(trifluoromethyl)benzenesulfonamide;
N-[1-(3,4-difluorobenzoyl)piperidin-4-yl]-5-(phenylsulfonyl)-2-(trifluoromethyl)benzenesulfonamide;
N-[1-(3,5-difluorobenzoyl)piperidin-4-yl]-5-(phenylsulfonyl)-2-(trifluoromethyl)benzenesulfonamide;
N-[1-(2,6-dimethoxybenzoyl)piperidin-4-yl]-5-(phenylsulfonyl)-2-(trifluoromethyl)benzenesulfonamide;
N-[1-(2,4-dimethoxybenzoyl)piperidin-4-yl]-5-(phenylsulfonyl)-2-(trifluoromethyl)benzenesulfonamide;
5-(phenylsulfonyl)-N-{1-[4-(trifluoromethoxy)benzoyl]piperidin-4-yl}-2-(trifluoromethyl)benzenesulfonamide;
N-{1-[2-fluoro-4-(trifluoromethyl)benzoyl]piperidin-4-yl}-5-(phenylsulfonyl)-2-(trifluoromethyl)benzenesulfonamide;
N-{1-[3-fluoro-4-(trifluoromethyl)benzoyl]piperidin-4-yl}-5-(phenylsulfonyl)-2-(trifluoromethyl)benzenesulfonamide;
N-[1-(3,4-dichlorobenzoyl)piperidin-4-yl]-5-(phenylsulfonyl)-2-(trifluoromethyl)benzenesulfonamide
N-[1-(4-chlorobenzoyl)piperidin-4-yl]-5-(phenylsulfonyl)-2-(trifluoromethyl)benzenesulfonamide;
N-(1-isonicotinoylpiperidin-4-yl)-5-(phenylsulfonyl)-2-(trifluoromethyl)benzenesulfonamide;
N-[1-(2-chloro-6-methoxyisonicotinoyl)piperidin-4-yl]-5-(phenylsulfonyl)-2-(trifluoromethyl)benzenesulfonamide;
N-[1-(2-chloro-4-fluorobenzoyl)piperidin-4-yl]-5-(phenylsulfonyl)-2-(trifluoromethyl)benzenesulfonamide;
5-(phenylsulfonyl)-N-[1-(2,4,6-trifluorobenzoyl)piperidin-4-yl]-2-(trifluoromethyl)benzenesulfonamide;
N-[1-(4-tert-butylbenzoyl)piperidin-4-yl]-5-(phenylsulfonyl)-2-(trifluoromethyl)benzenesulfonamide;
N-(4-{[4-({[5-(phenylsulfonyl)-2-(trifluoromethyl)phenyl]sulfonyl}amino)piperidin-1yl]carbonyl}phenyl)acetamide;
5-(phenylsulfonyl)-2-(trifluoromethyl)-N-(1-{[4-(trifluoromethyl)phenyl]carbonothioyl}piperidin-4-yl)benzenesulfonamide;
N-[1-(4-tert-butylbenzoyl)piperidin-4-yl]-5-[(3-methoxyphenyl)sulfonyl]-2-(trifluoromethyl)benzenesulfonamide;
N-[1-(4-tert-butylbenzoyl)piperidin-4-yl]-5-[(3-chlorophenyl)sulfonyl]-2-(trifluoromethyl)benzenesulfonamide;
5-(phenylsulfonyl)-N-{1-[2-(trifluoromethoxy)benzoyl]piperidin-4-yl}-2-(trifluoromethyl)benzenesulfonamide;
N-(1-benzoylpiperidin-4-yl)-5-(phenylsulfonyl)-2-(trifluoromethyl)benzenesulfonamide;
N-[1-(4-tert-butylbenzoyl)pyrrolidin-3-yl]-5-(phenylsulfonyl)-2-(trifluoromethyl)benzenesulfonamide;
N-[1-(4-tert-butylbenzoyl)piperidin-4-yl]-5-[(3-hydroxyphenyl)sulfonyl]-2-(trifluoromethyl)benzenesulfonamide;
N-[1-(4-benzoylbenzoyl)piperidin-4-yl]-5-(phenylsulfonyl)-2-(trifluoromethyl)benzenesulfonamide;
N-[1-(3-benzoylbenzoyl)piperidin-4-yl]-5-(phenylsulfonyl)-2-(trifluoromethyl)benzenesulfonamide;
1-Isopropyl-4-(phenylsulfonyl)benzene;
2-Isopropyl-N-[1-(2-methoxybenzoyl)piperidin-4-yl]-5-(phenylsulfonyl)benzenesulfonamide;
N-[1-(3-Fluorobenzoyl)piperidin-4-yl]-2-isopropyl-5-(phenylsulfonyl)benzenesulfonamide;
N-[1-(4-Fluorobenzoyl)piperidin-4-yl]-2-isopropyl-5-(phenylsulfonyl)benzenesulfonamide;
N-[1-(2-Fluorobenzoyl)piperidin-4-yl]-2-isopropyl-5-(phenylsulfonyl)benzenesulfonamide;
2-Isopropyl-5-(phenylsulfonyl)-N-{1-[4-(trifluoromethyl)benzoyl]piperidin-4-yl}benzenesulfonamide;
2-Isopropyl-N-[1-(1-naphthoyl)piperidin-4-yl]-5-(phenylsulfonyl)benzenesulfonamide;
2-Isopropyl-N-[1-(2-naphthoyl)piperidin-4-yl]-5-(phenylsulfonyl)benzenesulfonamide;
N-[1-(3-Cyanobenzoyl)piperidin-4-yl-2-isopropyl-5-(phenylsulfonyl)benzenesulfonamide;
N-[1-(4-Cyanobenzoyl)piperidin-4-yl]-2-isopropyl-5-(phenylsulfonyl)benzenesulfonamide;
N-[1-(4-tert-Butylbenzoyl)piperidin-4-yl]-2-isopropyl-5-(phenylsulfonyl)benzenesulfonamide;
N-[1-(2-Ethoxy-1-naphthoyl)piperidin-4-yl]-2-isopropyl-5-(phenylsulfonyl)benzenesulfonamide;
N-[1-(2-chloro-6-methylisonicotinoyl)piperidin-4-yl]-5-(phenylsulfonyl)-2-(trifluoromethyl)benzenesulfonamide;
N-[1-(2,6-dichloroisonicotinoyl)piperidin-4-yl]-5-(phenylsulfonyl)-2-(trifluoromethyl)benzenesulfonamide;
N-{1-[4-(dimethylamino)benzoyl]piperidin-4-yl}-5-(phenylsulfonyl)-2-(trifluoromethyl)benzenesulfonamide;
N-{1-[(6-chloropyridin-3-yl)carbonyl]piperidin-4-yl}-5-(phenylsulfonyl)-2-(trifluoromethyl)benzenesulfonamide;
N-{1-[(2,5-dichloropyridin-3-yl)carbonyl]piperidin-4-yl}-5-(phenylsulfonyl)-2-(trifluoromethyl)benzenesulfonamide;
N-[1-(2-chloroisonicotinoyl)piperidin-4-yl]-5-(phenylsulfonyl)-2-(trifluoromethyl)benzenesulfonamide;
5-(phenylsulfonyl)-N-{1-[(6-pyrrolidin-1-ylpyridin-3-yl)carbonyl]piperidin-4-yl}-2-(trifluoromethyl)benzenesulfonamide;
N-(1-{[6-(dimethylamino)pyridin-3-yl]carbonyl}piperidin-4-yl)-5-(phenylsulfonyl)-2-(trifluoromethyl)benzenesulfonamide;
N-{1-[(6-oxo-1-{[5-(phenylsulfonyl)-2-(trifluoromethyl)phenyl]sulfonyl}-1,6-dihydropyridin-3-yl)carbonyl]piperidin-4-yl}-5-(phenylsulfonyl)-2-(trifluoromethyl)benzenesulfonamide;
N-{1-[(6-phenylpyridin-3-yl)carbonyl]piperidin-4-yl}-5-(phenylsulfonyl)-2-(trifluoromethyl)benzenesulfonamide;
N-{1-[(6-morpholin-4-ylpyridin-3-yl)carbonyl]piperidin-4-yl}-5-(phenylsulfonyl)-2-(trifluoromethyl)benzenesulfonamide;
5-(phenylsulfonyl)-N-[1-(2-pyrrolidin-1-ylisonicotinoyl)piperidin-4-yl]-2-(trifluoromethyl)benzenesulfonamide;
N-{1-[(6-oxo-1,6-dihydropyridin-3-yl)carbonyl]piperidin-4-yl}-5-(phenylsulfonyl)-2-(trifluoromethyl)benzenesulfonamide;
5-(phenylsulfonyl)-N-[1-(pyridin-3-ylcarbonyl)piperidin-4-yl]-2-(trifluoromethyl)benzenesulfonamide;
5-(phenylsulfonyl)-N-[1-(pyridin-2-ylcarbonyl)piperidin-4-yl]-2-(trifluoromethyl)benzenesulfonamide;
N-{1-[4-(methylthio)benzoyl]piperidin-4-yl}-5-(phenylsulfonyl)-2-(trifluoromethyl)benzenesulfonamide;
5-(phenylsulfonyl)-2-(trifluoromethyl)-N-(1-{[6-(trifluoromethyl)pyridin-3-yl]carbonyl}piperidin-4-yl)benzenesulfonamide;
N-{1-[4-(methylsulfinyl)benzoyl]piperidin-4-yl}-5-(phenylsulfonyl)-2-(trifluoromethyl)benzenesulfonamide; or
5-(phenylsulfonyl)-N-[1-(1,3-thiazol-4-ylcarbonyl)piperidin-4-yl]-2-(trifluoromethyl)benzenesulfonamide.

39. The compound of claim 1 which is
tert-butyl 4-({[5-(phenylsulfonyl)-2-(trifluoromethyl)phenyl]sulfonyl}amino)piperidine-1-carboxylate;
tert-butyl (3S)-3-({[5-(phenylsulfonyl)-2-(trifluoromethyl)phenyl]sulfonyl}amino)piperidine-1-carboxylate;
tert-butyl (3R)-3-({[5-(phenylsulfonyl)-2-(trifluoromethyl)phenyl]sulfonyl}amino)piperidine-1-carboxylate;
tert-butyl 4-[({5-[(4-fluorophenyl)sulfonyl]-2-methylphenyl}sulfonyl)amino]piperidine-1-carboxylate;
tert-butyl (3S)-3-({[5-(phenylsulfonyl)-2-(trifluoromethyl)phenyl]sulfonyl}amino)pyrrolidine-1-carboxylate;
tert-butyl (3R)-3-({[5-(phenylsulfonyl)-2-(trifluoromethyl)phenyl]sulfonyl}amino)pyrrolidine-1-carboxylate;
tert-butyl 4-({[5-[(4-fluorophenyl)sulfonyl]-2-(trifluoromethyl)phenyl]sulfonyl}amino)piperidine-1-carboxylate;
tert-butyl 4-({[2-ethyl-5-(phenylsulfonyl)phenyl]sulfonyl}amino)piperidine-1-carboxylate;
tert-butyl 4-({[2,3-dimethyl-5-(phenylsulfonyl)phenyl]sulfonyl}amino)piperidine-1-carboxylate;
tert-butyl 4-({[2,4-dimethyl-5-(phenylsulfonyl)phenyl]sulfonyl}amino)piperidine-1-carboxylate;
tert-butyl 4-({[2-isopropyl-5-(phenylsulfonyl)phenyl]sulfonyl}amino)piperidine-1-carboxylate; or
4-{[3-({[1-(tert-butoxycarbonyl)piperidin-4-yl]amino}sulfonyl)-4-(trifluoromethyl)phenyl]sulfonyl}benzoic acid.

40. The compound of claim 1 which is
4-oxo-4-[4-({[5-(phenylsulfonyl)-2-(trifluoromethyl)phenyl]sulfonyl}amino)piperidin-1yl]butanoic acid;
4-oxo-4-[3-({[5-(phenylsulfonyl)-2-(trifluoromethyl)phenyl]sulfonyl}amino)pyrrolidin-1-yl]butanoic acid;
5-oxo-5-[4-({[5-(phenylsulfonyl)-2-(trifluoromethyl)phenyl]sulfonyl}amino)piperidin-1-yl]pentanoic acid;
5-oxo-5-[3-({[5-(phenylsulfonyl)-2-(trifluoromethyl)phenyl]sulfonyl}amino)pyrrolidin-1yl]pentanoic acid;
N-[1-(N,N-dimethylglycyl)piperidin-4-yl]-5-(phenylsulfonyl)-2-(trifluoromethyl)benzenesulfonamide;
tert-butyl 4-{2-oxo-2-[4-({[5-(phenylsulfonyl)-2-(trifluoromethyl)phenyl]sulfonyl}amino)piperidin-1-yl]ethyl}piperidine-1-carboxylate;
5-(phenylsulfonyl)-N-[1-(piperidin-4-ylacetyl)piperidin-4-yl]-2-(trifluoromethyl)benzenesulfonamide;
N-[1-(N-methylglycyl)piperidin-4-yl]-5-(phenylsulfonyl)-2-(trifluoromethyl)benzenesulfonamide;
5-(phenylsulfonyl)-N-[1-(pyrrolidin-1-ylacetyl)piperidin-4-yl]-2-(trifluoromethyl)benzenesulfonamide;
N-[1-(morpholin-4-ylacetyl)piperidin-4-yl]-5-(phenylsulfonyl)-2-(trifluoromethyl)benzenesulfonamide;
5-(phenylsulfonyl)-N-[1-(piperazin-1-ylacetyl)piperidin-4-yl]-2-(trifluoromethyl)benzenesulfonamide;
N-{1-[3-(methylthio)propanoyl]piperidin-4-yl}-5-(phenylsulfonyl)-2-(trifluoromethyl)benzenesulfonamide;
N-(1-[3-(methylsulfinyl)propanoyl]piperidin-4-yl}-5-(phenylsulfonyl)-2-(trifluoromethyl)benzenesulfonamide; or
N-[1-(1H-imidazol-1-ylacetyl)piperidin-4-yl]-5-(phenylsulfonyl)-2-(trifluoromethyl)benzenesulfonamide.

41. The compound of claim 1 which is
4-[({5-[(4-fluorophenyl)sulfonyl]-2-isopropylphenyl}sulfonyl)amino]-N-1-naphthylpiperidine-1-carbothioamide;
N-(2-fluorophenyl)-4-[({5-[(4-fluorophenyl)sulfonyl]-2-isopropylphenyl}sulfonyl)amino]piperidine-1-carbothioamide;
4-[({5-[(4-fluorophenyl)sulfonyl]-2-isopropylphenyl}sulfonyl)amino]-N-(2-methylphenyl)piperidine-1-carbothioamide;
ethyl ({4-[({5-[(4-fluorophenyl)sulfonyl]-2-isopropylphenyl}sulfonyl)amino]piperidin-1-yl}carbonothioyl)carbamate;
N-butyl-4-[({5-[(4-fluorophenyl)sulfonyl]-2-isopropylphenyl}sulfonyl)amino]piperidine-1-carbothioamide;
4-[({5-[(4-fluorophenyl)sulfonyl]-2-isopropylphenyl}sulfonyl)amino]-N-(4-methoxyphenyl)piperidine-1-carbothioamide;
methyl 4-[({4-[({5-[(4-fluorophenyl)sulfonyl]-2-isopropylphenyl}sulfonyl)amino]piperidin-1-yl}carbonothioyl)amino]benzoate;
methyl N-({4-[({5-[(4-fluorophenyl)sulfonyl]-2-isopropylphenyl}sulfonyl)amino]piperidin-1-yl}carbonothioyl)glycinate;
4-[({5-[(4-fluorophenyl)sulfonyl]-2-isopropylphenyl}sulfonyl)amino]-N-(2-morpholin-4-ylethyl)piperidine-1-carbothioamide;
4-[({5-[(4-fluorophenyl)sulfonyl]-2-isopropylphenyl}sulfonyl)amino]-N-(3-nitrophenyl)piperidine-1-carbothioamide;
3-[({4-[({5-[(4-fluorophenyl)sulfonyl]-2-isopropylphenyl}sulfonyl)amino]piperidin-1-yl}carbonothioyl)amino]benzoic acid;
4-[({5-[(4-fluorophenyl)sulfonyl]-2-isopropylphenyl}sulfonyl)amino]-N-pyridin-3-yl)piperidine-1-carbothioamide;
4-[({5-[(4-fluorophenyl)sulfonyl]-2-isopropylphenyl}sulfonyl)amino]-N-[4-(trifluoromethyl)phenyl]piperidine-1-carbothioamide;
N-(4-tert-butylphenyl)-4-({[5-(phenylsulfonyl)-2-(trifluoromethyl)phenyl]sulfonyl}amino)piperidine-1-carboxamide; or
3-({[4-({[5-(phenylsulfonyl)-2-(trifluoromethyl)phenyl]sulfonyl}amino)piperidin-1-yl]carbonothioyl}amino)benzoic acid.

42. The compound of claim 1 which is
tert-butyl 4-{[4-({[5-(phenylsulfonyl)-2-(trifluoromethyl)phenyl]sulfonyl}amino)piperidin-1yl]carbonyl}piperidine-1-carboxylate;
5-(phenylsulfonyl)-N-[1-(piperidin-4-ylcarbonyl)piperidin-4-yl]-2-(trifluoromethyl)benzenesulfonamide;
tert-butyl (2S)-2-{[4-({[5-(phenylsulfonyl)-2-(trifluoromethyl)phenyl]sulfonyl}amino)piperidin-1-yl]carbonyl}pyrrolidine-1-carboxylate;
5-(phenylsulfonyl)-N-(1-L-prolylpiperidin-4-yl)-2-(trifluoromethyl)benzenesulfonamide;
tert-butyl (2R)-2-{[4-({[5-(phenylsulfonyl)-2-(trifluoromethyl)phenyl]sulfonyl}amino)piperidin-1-yl]carbonyl}pyrrolidine-1-carboxylate;
5-(phenylsulfonyl)-N-(1-D-prolylpiperidin-4-yl)-2-(trifluoromethyl)benzenesulfonamide;
N-[1-(1-acetyl-L-prolyl)piperidin-4-yl]-5-(phenylsulfonyl)-2-(trifluoromethyl)benzenesulfonamide;
tert-butyl (5S)-2-oxo-5-{[4-({[5-(phenylsulfonyl)-2-(trifluoromethyl)phenyl]sulfonyl}amino)piperidin-1-yl]carbonyl}pyrrolidine-1-carboxylate;
N-[1-(5-oxo-L-prolyl)piperidin-4-yl]-5-(phenylsulfonyl)-2-(trifluoromethyl)benzenesulfonamide;
N-[1-(1-methyl-L-prolyl)piperidin-4-yl]-5-(phenylsulfonyl)-2-(trifluoromethyl)benzenesulfonamide;
tert-butyl (4R)-4-{[4-({[5-(phenylsulfonyl)-2-(trifluoromethyl)phenyl]sulfonyl}amino)piperidin-1-yl]carbonyl}-1,3-thiazolidine-3-carboxylate;
5-(phenylsulfonyl)-N-{1-[(4R)-1,3-thiazolidin-4-ylcarbonyl]piperidin-4-yl}-2-(trifluoromethyl)benzenesulfonamide;
tert-butyl (3R)-3-{[4-({[5-(phenylsulfonyl)-2-(trifluoromethyl)phenyl]sulfonyl}amino)piperidin-1-yl]carbonyl}pyrrolidine-1-carboxylate;
tert-butyl (3S)-3-{[4-({[5-(phenylsulfonyl)-2-(trifluoromethyl)phenyl]sulfonyl}amino)piperidin-1-yl]carbonyl}pyrrolidine-1-carboxylate;
5-(phenylsulfonyl)-N-{1-[(3R)-pyrrolidin-3-ylcarbonyl]piperidin-4-yl}-2-(trifluoromethyl)benzenesulfonamide;
5-(phenylsulfonyl)-N-{1-[(3S)-pyrrolidin-3-ylcarbonyl]piperidin-4-yl}-2-(trifluoromethyl)benzenesulfonamide;
tert-butyl (4R)-4-{[4-({[5-(phenylsulfonyl)-2-(trifluoromethyl)phenyl]sulfonyl}amino)piperidin-1-yl]carbonyl}-1,3-thiazolidine-3-carboxylate 1-oxide;
N-(1-{[(3R)-1-acetylpyrrolidin-3-yl]carbonyl}piperidin-4-yl)-5-(phenylsulfonyl)-2-(trifluoromethyl)benzenesulfonamide;
N-(1-{[(3S)-1-acetylpyrrolidin-3-yl]carbonyl}piperidin-4-yl)-5-(phenylsulfonyl)-2-(trifluoromethyl)benzenesulfonamide;
N-[1-(1-isobutyryl-L-prolyl)piperidin-4-yl]-5-(phenylsulfonyl)-2-(trifluoromethyl)benzenesulfonamide;
N-{1-[1-(2,2-dimethylpropanoyl)-L-prolyl]piperidin-4-yl}-5-(phenylsulfonyl)-2-(trifluoromethyl)benzenesulfonamide;
N-{1-[1-(3,3-dimethylbutanoyl)-L-prolyl]piperidin-4-yl}-5-(phenylsulfonyl)-2-(trifluoromethyl)benzenesulfonamide;
N-{1-[1-(cyclohexylcarbonyl)-L-prolyl]piperidin-4-yl}-5-(phenylsulfonyl)-2-(trifluoromethyl)benzenesulfonamide;
N-{1-[1-(morpholin-4-ylcarbonyl)-L-prolyl]piperidin-4-yl}-5-(phenylsulfonyl)-2-(trifluoromethyl)benzenesulfonamide;
(2S)—N-(tert-butyl)-2-{[4-({[5-(phenylsulfonyl)-2-(trifluoromethyl)phenyl]sulfonyl}amino)piperidin-1-yl]carbonyl}pyrrolidine-1-carboxamide;
(2S)—N-phenyl-2-{[4-({[5-(phenylsulfonyl)-2-(trifluoromethyl)phenyl]sulfonyl}amino)piperidin-1-yl]carbonyl}pyrrolidine-1-carboxamide;
N-{1-[1-(methylsulfonyl)-L-prolyl]piperidin-4-yl}-5-(phenylsulfonyl)-2-(trifluoromethyl)benzenesulfonamide;
N-[1-(1-benzoyl-L-prolyl)piperidin-4-yl]-5-(phenylsulfonyl)-2-(trifluoromethyl)benzenesulfonamide;
N-(1-{1-[4-(dimethylamino)benzoyl]-L-prolyl}piperidin-4-yl)-5-(phenylsulfonyl)-2-(trifluoromethyl)benzenesulfonamide;
N-[1-(1-isonicotinoyl-L-prolyl)piperidin-4-yl]-5-(phenylsulfonyl)-2-(trifluoromethyl)benzenesulfonamide;
N-(1-{1-[(6-chloropyridin-3-yl)carbonyl]-L-prolyl}piperidin-4-yl)-5-(phenylsulfonyl)-2-(trifluoromethyl)benzenesulfonamide;
4-[((2S)-2-{[4-({[5-(phenylsulfonyl)-2-(trifluoromethyl)phenyl]sulfonyl}amino)piperidin-1-yl]carbonyl}pyrrolidin-1-yl)sulfonyl]benzoic acid;
N-[1-(N,N-dimethylglycyl-L-prolyl)piperidin-4-yl]-5-(phenylsulfonyl)-2-(trifluoromethyl)benzenesulfonamide;
N-[1-(1-benzyl-L-prolyl)piperidin-4-yl]-5-(phenylsulfonyl)-2-(trifluoromethyl)benzenesulfonamide;
N-{1-[1-(cyclohexylmethyl)-L-prolyl]piperidin-4-yl}-5-(phenylsulfonyl)-2-(trifluoromethyl)benzenesulfonamide;
N-{1-[1-(3,3-dimethylbutyl)-L-prolyl]piperidin-4-yl}-5-(phenylsulfonyl)-2-(trifluoromethyl)benzenesulfonamide;
(2S)—N-ethyl-2-{[4-({[5-(phenylsulfonyl)-2-(trifluoromethyl)phenyl]sulfonyl}amino)piperdin 1-yl]carbonyl}pyrrolidine-1-carboxamide;
(2S)—N,N-dimethyl-2-{[4-({[5-(phenylsulfonyl)-2-(trifluoromethyl)phenyl]sulfonyl}amino)piperidin-1-yl]carbonyl}pyrrolidine-1-carboxamide; or
tert-butyl (2S)-2-{[4-({[5-[(3-cyanophenyl)sulfonyl]-2-(trifluoromethyl)phenyl]sulfonyl}amino)piperidin-1-yl]carbonyl}pyrrolidine-1-carboxylate.

43. The compound of claim 1 which is
N-[1-(2-hydroxy-2-methylpropyl)piperidin-4-yl]-5-(phenylsulfonyl)-2-(trifluoromethyl)benzenesulfonamide;
5-(phenylsulfonyl)-2-(trifluoromethyl)-N-{1-[4-(trifluoromethyl)benzyl]piperidin-4-yl}benzenesulfonamide;
N-[1-(cyanomethyl)piperidin-4-yl]-5-(phenylsulfonyl)-2-(trifluoromethyl)benzenesulfonamide;
N-[1-(2-oxo-2-phenylethyl)piperidin-4-yl]-5-(phenylsulfonyl)-2-(trifluoromethyl)benzenesulfonamide;
5-(phenylsulfonyl)-2-(trifluoromethyl)-N-{1-[4-(trifluoromethyl)phenyl]piperidin-4-yl}benzenesulfonamide;
N-[1-(2-hydroxyethyl)piperidin-4-yl]-5-(phenylsulfonyl)-2-(trifluoromethyl)benzenesulfonamide;
2-Isopropyl-N-[(1R*,5S*)-8-methyl-8-azabicyclo[3.2.1]oct-3-yl]-5-(phenylsulfonyl)benzenesulfonamide monohydrochloride;
[4-({[5-(phenylsulfonyl)-2-(trifluoromethyl)phenyl]sulfonyl}amino)piperidin-1-yl]acetic acid;
2-[4-({[5-(phenylsulfonyl)-2-(trifluoromethyl)phenyl]sulfonyl}amino)piperidin-1-yl]acetamide;
5-(phenylsulfonyl)-N-[1-(2H-tetrazol-5-ylmethyl)piperidin-4-yl]-2-(trifluoromethyl)benzenesulfonamide;
3-[4-({[5-(phenylsulfonyl)-2-(trifluoromethyl)phenyl]sulfonyl}amino)piperidin-1-yl]propanoic acid;
3-[4-({[5-(phenylsulfonyl)-2-(trifluoromethyl)phenyl]sulfonyl}amino)piperidin-1-yl]benzoic acid;
4-[4-({[5-(phenylsulfonyl)-2-(trifluoromethyl)phenyl]sulfonyl}amino)piperidin-1-yl]benzoic acid;
N-[1-(3-cyanophenyl)piperidin-4-yl]-5-(phenylsulfonyl)-2-(trifluoromethyl)benzenesulfonamide;
2-[4-({[5-(phenylsulfonyl)-2-(trifluoromethyl)phenyl]sulfonyl}amino)piperidin-1-yl]propanamide;
N-[1-(2-morpholin-4-ylethyl)piperidin-4-yl]-5-(phenylsulfonyl)-2-(trifluoromethyl)benzenesulfonamide;
N-[(1R*,5S*)-8-Methyl-8-azabicyclo[3.2.1]oct-3-yl]-5-(phenylsulfonyl)-2-(trifluoromethyl)benzenesulfonamide;
(2R)-2-[4-({[5-(phenylsulfonyl)-2-(trifluoromethyl)phenyl]sulfonyl}amino)piperidin-1-yl]propanamide;
(2S)-2-[4-({[5-(phenylsulfonyl)-2-(trifluoromethyl)phenyl]sulfonyl}amino)piperidin-1-yl]propanamide;
methyl [4-({[5-(phenylsulfonyl)-2-(trifluoromethyl)phenyl]sulfonyl}amino)piperidin-1-yl]acetate;
N-methyl-2-[4-({[5-(phenylsulfonyl)-2-(trifluoromethyl)phenyl]sulfonyl}amino)piperidin-1-yl]acetamide;
N,N-dimethyl-2-[4-({[5-(phenylsulfonyl)-2-(trifluoromethyl)phenyl]sulfonyl}amino)piperidin-1yl]acetamide;
N-isopropyl-2-[4-({[5-(phenylsulfonyl)-2-(trifluoromethyl)phenyl]sulfonyl}amino)piperidin-1yl]acetamide; or
N-[1-(2-morpholin-4-yl-2-oxoethyl)piperidin-4-yl]-5-(phenylsulfonyl)-2-(trifluoromethyl)benzenesulfonamide.

44. The compound of claim 1 which is
N-{1-[(4-tert-butylphenyl)sulfonyl]piperidin-4-yl}-5-(phenylsulfonyl)-2-(trifluoromethyl)benzenesulfonamide;
5-(phenylsulfonyl)-N-[1-(phenylsulfonyl)piperidin-4-yl]-2-(trifluoromethyl)benzenesulfonamide;
3-{[4-({[5-(phenylsulfonyl)-2-(trifluoromethyl)phenyl]sulfonyl}amino)piperidin-1-yl]sulfonyl}benzoic acid;
4-{[4-({[5-(phenylsulfonyl)-2-(trifluoromethyl)phenyl]sulfonyl}amino)piperidin-1-yl]sulfonyl}benzoic acid;
N-{1-[(4-hydroxyphenyl)sulfonyl]piperidin-4-yl}-5-(phenylsulfonyl)-2-(trifluoromethyl)benzenesulfonamide;
methyl 3-{[4-({[5-(phenylsulfonyl)-2-(trifluoromethyl)phenyl]sulfonyl}amino)piperidin-1yl]sulfonyl}benzoate;
N-{1-[(3-cyanophenyl)sulfonyl]piperidin-4-yl}-5-(phenylsulfonyl)-2-(trifluoromethyl)benzenesulfonamide; or
5-(phenylsulfonyl)-N-(1-{[3-(2H-tetrazol-5-yl)phenyl]sulfonyl}piperidin-4-yl)-2-(trifluoromethyl)benzenesulfonamide.

45. The compound of claim 1 which is
5-(phenylsulfonyl)-N-(2-pyridin-3-ylethyl)-2-(trifluoromethoxy)benzenesulfonamide;
2-isopropyl-5-(phenylsulfonyl)-N-(2-pyridin-2-ylethyl)benzenesulfonamide;
2-isopropyl-5-(phenylsulfonyl)-N-(2-pyridin-3-ylethyl)benzenesulfonamide;
5-(phenylsulfonyl)-N-(2-pyridin-3-ylethyl)-2-(trifluoromethyl)benzenesulfonamide;
1-Methoxy-4-(phenylsulfonyl)benzene;
N-[2-(1-oxidopyridin-3-yl)ethyl]-5-(phenylsulfonyl)-2-(trifluoromethyl)benzenesulfonamide;
5-[(4-fluorophenyl)sulfonyl]-N-(2-hydroxy-2-pyridin-2-ylethyl)-2-isopropylbenzenesulfonamide;
N-(2-hydroxy-2-pyridin-2-ylethyl)-2-isopropyl-5-(phenylsulfonyl)benzene-sulfonamide;
N-(2-hydroxy-2-pyridin-2-ylethyl)-2,4-dimethyl-5-(phenylsulfonyl)benzenesulfonamide;
5-[(4-fluorophenyl)sulfonyl]-N-(2-hydroxy-2-pyridin-3-ylethyl)-2-isopropylbenzenesulfonamide;
N-(2-hydroxy-2-pyridin-3-ylethyl)-2-isopropyl-5-(phenylsulfonyl)benzenesulfonamide;
N-(2-hydroxy-2-pyridin-3-ylethyl)-2,4-dimethyl-5-(phenylsulfonyl)benzenesulfonamide;
N-[2-(1H-Imidazol-1-yl)ethyl]-5-(phenylsulfonyl)-2-(trifluoromethyl)benzenesulfonamide;
5-[(4-fluorophenyl)sulfonyl]-N-(2-hydroxy-2-pyridin-3-ylethyl)-2-methylbenzenesulfonamide;
N-(2-hydroxy-2-pyridin-3-ylethyl)-5-(phenylsulfonyl)-2-(trifluoromethyl)benzenesulfonamide;
5-[(3-methoxyphenyl)sulfonyl]-N-(2-pyridin-3-ylethyl)-2-(trifluoromethyl)benzenesulfonamide;
5-[(3-chlorophenyl)sulfonyl]-N-(2-pyridin-3-ylethyl)-2-(trifluoromethyl)benzenesulfonamide;
5-[(4-fluorophenyl)sulfonyl]-N-(2-hydroxy-2-pyridin-2-ylethyl)-2-methylbenzenesulfonamide;
N-(2-hydroxy-2-pyridin-2-ylethyl)-5-(phenylsulfonyl)-2-(trifluoromethyl)benzenesulfonamide;
5-(phenylsulfonyl)-N-(2-pyridin-4-ylethyl)-2-(trifluoromethyl)benzenesulfonamide;
5-[(4-hydroxyphenyl)sulfonyl]-N-(2-pyridin-3-ylethyl)-2-(trifluoromethyl)benzenesulfonamide;
5-[(4-fluorophenyl)sulfonyl]-N-[2-(1H-imidazol-1-yl)ethyl]-2-(trifluoromethyl)benzenesulfonamide;
5-[(2-fluorophenyl)sulfonyl]-N-[2-(1H-imidazol-1-yl)ethyl]-2-(trifluoromethyl)benzenesulfonamide;
5-(Phenylsulfonyl)-2-propyl-N-(2-pyridin-4-ylethyl)benzenesulfonamide;
5-(phenylsulfonyl)-N-[2-(2H-tetrazol-5-yl)ethyl]-2-(trifluoromethyl)benzenesulfonamide;
5-[(4-Fluorophenyl)sulfonyl]-2-propyl-N-(2-pyridin-3-ylethyl)benzenesulfonamide;
5-[(4-Fluorophenyl)sulfonyl]-2-propyl-N-(2-pyridin-4-ylethyl)benzenesulfonamide;
5-(Phenylsulfonyl)-2-propyl-N-(2-pyridin-3-ylethyl)benzenesulfonamide;
2-isopropyl-5-(phenylsulfonyl)-N-(pyridin-4-ylmethyl)benzenesulfonamide;
5-(phenylsulfonyl)-N-(pyridin-4-ylmethyl)-2-(trifluoromethyl)benzenesulfonamide;
N-[(6-chloropyridin-3-yl)methyl]-5-(phenylsulfonyl)-2-(trifluoromethyl)benzenesulfonamide;
5-(phenylsulfonyl)-N-[(6-pyrrolidin-1-ylpyridin-3-yl)methyl]-2-(trifluoromethyl)benzenesulfonamide; or
N-[(6-morpholin-4-ylpyridin-3-yl)methyl]-5-(phenylsulfonyl)-2-(trifluoromethyl)benzenesulfonamide.

46. The compound of claim 1 which is
2-chloro-N-[(1R*,2R*)-2-hydroxy-1-methyl-2-phenylethyl]-5-(phenylsulfonyl)benzenesulfonamide;
N-(2-hydroxy-2-phenylethyl)-5-(phenylsulfonyl)-2-(trifluoromethyl)benzenesulfonamide;
N-[(1R*,2R*)-2-hydroxy-1-methyl-2-phenylethyl]-5-(phenylsulfonyl)-2-(trifluoromethyl)benzenesulfonamide;
N-[(1S,2R)-2-hydroxy-1-methyl-2-phenylethyl]-5-(phenylsulfonyl)-2-(trifluoromethyl)benzenesulfonamide;
N-[(1R,2S)-2-hydroxy-1-methyl-2-phenylethyl]-5-(phenylsulfonyl)-2-(trifluoromethyl)benzenesulfonamide;
N-[(1S)-1-benzyl-2-hydroxyethyl]-5-(phenylsulfonyl)-2-(trifluoromethyl)benzenesulfonamide;
N-[(1S)-2-hydroxy-1-(1H-indol-3-ylmethyl)ethyl]-5-(phenylsulfonyl)-2-(trifluoromethyl)benzenesulfonamide;
5-[(4-fluorophenyl)sulfonyl]-N-(2-hydroxy-2-phenylethyl)-2-isopropylbenzenesulfonamide;
N-(2-hydroxy-2-phenylethyl)-2-isopropyl-5-(phenylsulfonyl)benzenesulfonamide;
N-[(2R)-2-hydroxy-2-phenylethyl]-5-(phenylsulfonyl)-2-(trifluoromethyl)benzenesulfonamide;
N-[(2S)-2-hydroxy-2-phenylethyl]-5-(phenylsulfonyl)-2-(trifluoromethyl)benzenesulfonamide;
4-[2-({[5-(phenylsulfonyl)-2-(trifluoromethyl)phenyl]sulfonyl}amino)ethyl]benzoic acid;
N-[2-(4-aminophenyl)ethyl]-5-(phenylsulfonyl)-2-(trifluoromethyl)benzenesulfonamide;
N-[2-(4-methoxyphenyl)ethyl]-5-(phenylsulfonyl)-2-(trifluoromethyl)benzenesulfonamide;
N-(4-aminobenzyl)-5-(phenylsulfonyl)-2-(trifluoromethyl)benzenesulfonamide;
N-[2-(4-hydroxyphenyl)ethyl]-5-(phenylsulfonyl)-2-(trifluoromethyl)benzenesulfonamide;
methyl 4-[({[5-(phenylsulfonyl)-2-(trifluoromethyl)phenyl]sulfonyl}amino)methyl]benzoate;
methyl 4-[({[2-isopropyl-5-(phenylsulfonyl)phenyl]sulfonyl}amino)methyl]benzoate;
N-[2-(4-bromophenyl)ethyl]-5-(phenylsulfonyl)-2-(trifluoromethyl)benzenesulfonamide;
3-[({4-[({[5-(phenylsulfonyl)-2-(trifluoromethyl)phenyl]sulfonyl}amino)methyl]phenyl}amino)sulfonyl]benzoic acid;
N-(4-bromobenzyl)-5-(phenylsulfonyl)-2-(trifluoromethyl)benzenesulfonamide;
N-[2-(4-cyanophenyl)ethyl]-5-(phenylsulfonyl)-2-(trifluoromethyl)benzenesulfonamide;
methyl 4-[2-({[5-(phenylsulfonyl)-2-(trifluoromethyl)phenyl]sulfonyl}amino)ethyl]benzoate;
4′-[({[5-(phenylsulfonyl)-2-(trifluoromethyl)phenyl]sulfonyl}amino)methyl]biphenyl-3-carboxylic acid;
4′-[({[5-(phenylsulfonyl)-2-(trifluoromethyl)phenyl]sulfonyl}amino)methyl]biphenyl-4-carboxylic acid;
5-(phenylsulfonyl)-N-{2-[4-(2H-tetrazol-5-yl)phenyl]ethyl}-2-(trifluoromethyl)benzenesulfonamide;
N-(2-{4-[(methylsulfonyl)amino]phenyl}ethyl)-5-(phenylsulfonyl)-2-(trifluoromethyl)benzenesulfonamide;
N-(3-bromobenzyl)-5-(phenylsulfonyl)-2-(trifluoromethyl)benzenesulfonamide;
3′-[({[5-(phenylsulfonyl)-2-(trifluoromethyl)phenyl]sulfonyl}amino)methyl]biphenyl-3-carboxylic acid;
3′-[({[5-(phenylsulfonyl)-2-(trifluoromethyl)phenyl]sulfonyl}amino)methyl]biphenyl-4-carboxylic acid;
N-[2-(4-{[amino(imino)methyl]amino}phenyl)ethyl]-5-(phenylsulfonyl)-2-(trifluoromethyl)benzenesulfonamide;
N-[4-(dimethylamino)benzyl]-5-(phenylsulfonyl)-2-(trifluoromethyl)benzenesulfonamide; or
N-(2,4-dimethoxybenzyl)-5-(phenylsulfonyl)-2-(trifluoromethyl)benzenesulfonamide.

47. The compound of claim 1 which is
tert-Butyl [2-({[5-(phenylsulfonyl)-2-(trifluoromethyl)phenyl]sulfonyl}amino)ethyl]carbamate;
N-(2-Aminoethyl)-5-(phenylsulfonyl)-2-(trifluoromethyl)benzenesulfonamide hydrochloride;
N-[2-({[5-(Phenylsulfonyl)-2-(trifluoromethyl)phenyl]sulfonyl}amino)ethyl]benzamide;
4-Methyl-N-[2-({[5-(phenylsulfonyl)-2-(trifluoromethyl)phenyl]sulfonyl}amino)ethyl]benzamide;
4-tert-Butyl-N-[2-({[5-(phenylsulfonyl)-2-(trifluoromethyl)phenyl]sulfonyl}amino)ethyl]benzamide;
4-Fluoro-N-[2-({[5-(phenylsulfonyl)-2-(trifluoromethyl)phenyl]sulfonyl}amino)ethyl]benzamide;
4-Chloro-N-[2-({[5-(phenylsulfonyl)-2-(trifluoromethyl)phenyl]sulfonyl}amino)ethyl]benzamide;
4-Bromo-N-[2-({[5-(phenylsulfonyl)-2-(trifluoromethyl)phenyl]sulfonyl}amino)ethyl]benzamide;
4-Methoxy-N-[2-({[5-(phenylsulfonyl)-2-(trifluoromethyl)phenyl]sulfonyl}amino)ethyl]benzamide;
N-[2-({[5-(Phenylsulfonyl)-2-(trifluoromethyl)phenyl]sulfonyl}amino)ethyl]-4-(trifluoromethyl)benzamide;
N-[2-({[5-(Phenylsulfonyl)-2-(trifluoromethyl)phenyl]sulfonyl}amino)ethyl]-4-(trifluoromethoxy)benzamide;
N-[2-({[5-(Phenylsulfonyl)-2-(trifluoromethyl)phenyl]sulfonyl}amino)ethyl]isonicotinamide;
tert-Butyl methyl[2-({[5-(phenylsulfonyl)-2-(trifluoromethyl)phenyl]sulfonyl}amino)ethyl]carbamate;
N-[2-(Methylamino)ethyl]-5-(phenylsulfonyl)-2-(trifluoromethyl)benzenesulfonamide hydrochloride;
N-Methyl-N-[2-({[5-(phenylsulfonyl)-2-(trifluoromethyl)phenyl]sulfonyl}amino)ethyl]benzamide;
4-Methoxy-N-methyl-N-[2-({[5-(phenylsulfonyl)-2-(trifluoromethyl)phenyl]sulfonyl}amino)ethyl]benzamide;
N-{2-[(anilinocarbonyl)(methyl)amino]ethyl}-5-(phenylsulfonyl)-2-(trifluoromethyl)benzenesulfonamide;
N-(2-{methyl[(pyridin-3-ylamino)carbonyl]amino}ethyl)-5-(phenylsulfonyl)-2-(trifluoromethyl)benzenesulfonamide;
N-{2-[{[(2,4-dimethoxyphenyl)amino]carbonyl}(methyl)amino]ethyl}-5-(phenylsulfonyl)-2-(trifluoromethyl)benzenesulfonamide;
N-{2-[[(tert-butylamino)carbonyl](methyl)amino]ethyl}-5-(phenylsulfonyl)-2-(trifluoromethyl)benzenesulfonamide;
N-{2-[{[(4-methoxyphenyl)amino]carbonyl}(methyl)amino]ethyl}-5-(phenylsulfonyl)-2-(trifluoromethyl)benzenesulfonamide;
N-{2-[[(butylamino)carbonyl](methyl)amino]ethyl}-5-(phenylsulfonyl)-2-(trifluoromethyl)benzenesulfonamide
N-{2-[{[(2,4-difluorophenyl)amino]carbonyl}(methyl)amino]ethyl}-5-(phenylsulfonyl)-2-(trifluoromethyl)benzenesulfonamide;
N-methyl-N-[2-({[5-(phenylsulfonyl)-2-(trifluoromethyl)phenyl]sulfonyl}amino)ethyl]pyrrolidine-1-carboxamide;
N-{2-[[(diethylamino)carbonyl](methyl)amino]ethyl}-5-(phenylsulfonyl)-2-(trifluoromethyl)benzenesulfonamide;
N-methyl-N-[2-({[5-(phenylsulfonyl)-2-(trifluoromethyl)phenyl]sulfonyl}amino)ethyl]morpholine-4-carboxamide;
N-[2-(methyl{[methyl(phenyl)amino]carbonyl}amino)ethyl]-5-(phenylsulfonyl)-2-(trifluoromethyl)benzenesulfonamide;
N-Methyl-N-[2-({[5-(phenylsulfonyl)-2-(trifluoromethyl)phenyl]sulfonyl}amino)ethyl]-2-furamide;
4-tert-Butyl-N-methyl-N-[2-({[5-(phenylsulfonyl)-2-(trifluoromethyl)phenyl]sulfonyl}amino)ethyl]benzamide;
N-Methyl-N-[2-({[5-(phenylsulfonyl)-2-(trifluoromethyl)phenyl]sulfonyl}amino)ethyl]-2-(trifluoromethoxy)benzamide;
N-methyl-N-[2-({[5-(phenylsulfonyl)-2-(trifluoromethyl)phenyl]sulfonyl}amino)ethyl]cyclohexanecarboxamide;
3-Fluoro-N-methyl-N-[2-({[5-(phenylsulfonyl)-2-(trifluoromethyl)phenyl]sulfonyl}amino)ethyl]-4-(trifluoromethyl)benzamide;
Methyl 4-({methyl[2-({[5-(phenylsulfonyl)-2-(trifluoromethyl)phenyl]sulfonyl}amino)ethyl]amino}carbonyl)benzoate;
N-Methyl-N-[2-({[5-(phenylsulfonyl)-2-(trifluoromethyl)phenyl]sulfonyl}amino)ethyl]nicotinamide;
N-Methyl-N-[2-({[5-(phenylsulfonyl)-2-(trifluoromethyl)phenyl]sulfonyl}amino)ethyl]isonicotinamide;
2-Chloro-N-methyl-N-[2-({[5-(phenylsulfonyl)-2-(trifluoromethyl)phenyl]sulfonyl}amino)ethyl]nicotinamide;
N,2,2-trimethyl-N-[2-({[5-(phenylsulfonyl)-2-(trifluoromethyl)phenyl]sulfonyl}amino)ethyl]propanamide;
2-ethyl-N-methyl-N-[2-({[5-(phenylsulfonyl)-2-(trifluoromethyl)phenyl]sulfonyl}amino)ethyl]butanamide;
butyl methyl[2-({[5-(phenylsulfonyl)-2-(trifluoromethyl)phenyl]sulfonyl}amino)ethyl]carbamate;
tert-butyl 4-[({methyl[2-({[5-(phenylsulfonyl)-2-(trifluoromethyl)phenyl]sulfonyl}amino)ethyl]amino}carbonyl)amino]piperidine-1-carboxylate;
2,2-dimethylpropyl methyl[2-({[5-(phenylsulfonyl)-2-(trifluoromethyl)phenyl]sulfonyl}amino)ethyl]carbamate;
isobutyl methyl[2-({[5-(phenylsulfonyl)-2-(trifluoromethyl)phenyl]sulfonyl)amino}ethyl]carbamate;
3-(trifluoromethyl)phenyl methyl[2-({[5-(phenylsulfonyl)-2-(trifluoromethyl)phenyl]sulfonyl}amino)ethyl]carbamate;
4-fluorophenyl methyl[2-({[5-(phenylsulfonyl)-2-(trifluoromethyl)phenyl]sulfonyl}amino)ethyl]carbamate;
4-bromophenyl methyl[2-({[5-(phenylsulfonyl)-2-(trifluoromethyl)phenyl]sulfonyl}amino)ethyl]carbamate;
N-(2-{methyl[(piperidin-4-ylamino)carbonyl]amino}ethyl)-5-(phenylsulfonyl)-2-(trifluoromethyl)benzenesulfonamide;
ethyl N-({methyl[2-({[5-(phenylsulfonyl)-2-(trifluoromethyl)phenyl]sulfonyl}amino)ethyl]amino}carbonyl)glycinate;
3-({methyl[2-({[5-(phenylsulfonyl)-2-(trifluoromethyl)phenyl]sulfonyl)amino)ethyl]amino}sulfonyl)benzoic acid;
tert-butyl 4-({methyl[2-({[5-(phenylsulfonyl)-2-(trifluoromethyl)phenyl]sulfonyl}amino)ethyl]amino}carbonyl)piperazine-1-carboxylate;
N-({methyl[2-({[5-(phenylsulfonyl)-2-(trifluoromethyl)phenyl]sulfonyl}amino)ethyl]amino}carbonyl)glycine;
4-{methyl[2-({[5-(phenylsulfonyl)-2-(trifluoromethyl)phenyl]sulfonyl}amino)ethyl]amino}-4-oxobutanoic acid;
N-methyl-N-[2-({[5-(phenylsulfonyl)-2-(trifluoromethyl)phenyl]sulfonyl}amino)ethyl]piperazine-1-carboxamide;
N-{2-[(2-hydroxyethyl)(methyl)amino]ethyl}-5-(phenylsulfonyl)-2-(trifluoromethyl)benzenesulfonamide;
methyl N-methyl-N-[2-({[5-(phenylsulfonyl)-2-(trifluoromethyl)phenyl]sulfonyl}amino)ethyl]glycinate;
ethyl N-methyl-N-[2-({[5-(phenylsulfonyl)-2-(trifluoromethyl)phenyl]sulfonyl}amino)ethyl]-□-alaninate;
tert-butyl (3S)-3-[({methyl[2-({[5-(phenylsulfonyl)-2-(trifluoromethyl)phenyl]sulfonyl}amino)ethyl]amino}carbonyl)amino]pyrrolidine-1-carboxylate;
tert-butyl methyl[3-({[5-(phenylsulfonyl)-2-(trifluoromethyl)phenyl]sulfonyl}amino)propyl]carbamate;
N-[3-(methylamino)propyl]-5-(phenylsulfonyl)-2-(trifluoromethyl)benzenesulfonamide;
N-[2-(methyl{[(3S)-pyrrolidin-3-ylamino]carbonyl}amino)ethyl]-5-(phenylsulfonyl)-2-(trifluoromethyl)benzenesulfonamide;
tert-butyl 4-[({methyl[3-({[5-(phenylsulfonyl)-2-(trifluoromethyl)phenyl]sulfonyl}amino)propyl]amino}carbonyl)amino]piperidine-1-carboxylate;
tert-butyl N-methyl-N-[2-({[5-(phenylsulfonyl)-2-(trifluoromethyl)phenyl]sulfonyl}amino)ethyl]-β-alaninate;
tert-butyl N-methyl-N-[2-({[5-(phenylsulfonyl)-2-(trifluoromethyl)phenyl]sulfonyl}amino)ethyl]glycinate;
N-(3-{methyl[(piperidin-4-ylamino)carbonyl]amino}propyl)-5-(phenylsulfonyl)-2-(trifluoromethyl)benzenesulfonamide;
tert-butyl 4-({methyl[3-({[5-(phenylsulfonyl)-2-(trifluoromethyl)phenyl]sulfonyl}amino)propyl]amino}carbonyl)piperazine-1-carboxylate;
N-methyl-N-[3-({[5-(phenylsulfonyl)-2-(trifluoromethyl)phenyl]sulfonyl}amino)propyl]piperazine-1-carboxamide;
4-{4-[({methyl[2-({[5-(phenylsulfonyl)-2-(trifluoromethyl)phenyl]sulfonyl}amino)ethyl]amino}carbonyl)amino]piperidin-1-yl}-4-oxobutanoic acid;
N-methyl-N-[2-({[5-(phenylsulfonyl)-2-(trifluoromethyl)phenyl]sulfonyl}amino)ethyl]glycine;
N-methyl-N-[2-({[5-(phenylsulfonyl)-2-(trifluoromethyl)phenyl]sulfonyl}amino)ethyl]-β-alanine;
4-(bromomethyl)-N-methyl-N-[2-({[5-(phenylsulfonyl)-2-(trifluoromethyl)phenyl]sulfonyl}amino)ethyl]benzamide;
tert-butyl [3-({[5-(phenylsulfonyl)-2-(trifluoromethyl)phenyl]sulfonyl}amino)propyl]carbamate;
tert-butyl 4-[methyl({methyl[2-({[5-(phenylsulfonyl)-2-(trifluoromethyl)phenyl]sulfonyl}amino)ethyl]amino}carbonyl)amino]piperidine-1-carboxylate;
tert-butyl (3R)-3-[({methyl[2-({[5-(phenylsulfonyl)-2-(trifluoromethyl)phenyl]sulfonyl}amino)ethyl]amino}carbonyl)amino]pyrrolidine-1-carboxylate;
4-{methyl[3-({[5-(phenylsulfonyl)-2-(trifluoromethyl)phenyl]sulfonyl}amino)propyl]amino}-4-oxobutanoic acid;
N-(3-aminopropyl)-5-(phenylsulfonyl)-2-(trifluoromethyl)benzenesulfonamide;
dimethyl [4-({methyl[2-({[5-(phenylsulfonyl)-2-(trifluoromethyl)phenyl]sulfonyl}amino)ethyl]amino}carbonyl)benzyl]phosphonate;
N-[2-(methyl{[methyl(piperidin-4-yl)amino]carbonyl}amino)ethyl]-5-(phenylsulfonyl)-2-(trifluoromethyl)benzenesulfonamide;
N-[2-(methyl{[(3R)-pyrrolidin-3-ylamino]carbonyl}amino)ethyl]-5-(phenylsulfonyl)-2-(trifluoromethyl)benzenesulfonamide;
tert-butyl (3R)-3-[({methyl[2-({[5-(phenylsulfonyl)-2-(trifluoromethyl)phenyl]sulfonyl}amino)ethyl]amino}carbonyl)amino]piperidine-1-carboxylate;
tert-butyl (3S)-3-[({methyl[2-({[5-(phenylsulfonyl)-2-(trifluoromethyl)phenyl]sulfonyl}amino)ethyl]amino}carbonyl)amino]piperidine-1-carboxylate;
N-[2-(methyl{[(3R)-piperidin-3-ylamino]carbonyl}amino)ethyl]-5-(phenylsulfonyl)-2-(trifluoromethyl)benzenesulfonamide;
N-[2-(methyl{[(3S)-piperidin-3-ylamino]carbonyl}amino)ethyl]-5-(phenylsulfonyl)-2-(trifluoromethyl)benzenesulfonamide;
[4-({methyl[2-({[5-(phenylsulfonyl)-2-(trifluoromethyl)phenyl]sulfonyl}amino)ethyl]amino}carbonyl)benzyl]phosphonic acid;
tert-butyl 4-[methyl({[2-({[5-(phenylsulfonyl)-2-(trifluoromethyl)phenyl]sulfonyl}amino)ethyl]amino}carbonyl)amino]piperidine-1-carboxylate; or
tert-butyl 4-[({[2-({[5-(phenylsulfonyl)-2-(trifluoromethyl)phenyl]sulfonyl}amino)ethyl]amino}carbonyl)amino}piperidine-1-carboxylate.

48. The compound of claim 1 which is
N-(2,3-dihydro-1H-inden-2-yl)-5-(phenylsulfonyl)-2-(trifluoromethyl)benzenesulfonamide;
N-(2-hydroxy-2,3-dihydro-1H-inden-1-yl)-5-(phenylsulfonyl)-2-(trifluoromethyl)benzenesulfonamide;
N-(1-hydroxy-2,3-dihydro-1H-inden-2-yl)-5-(phenylsulfonyl)-2-(trifluoromethyl)benzenesulfonamide;
N-(5-methoxy-2,3-dihydro-1H-inden-2-yl)-5-(phenylsulfonyl)-2-(trifluoromethyl)benzenesulfonamide;
N-(5-hydroxy-2,3-dihydro-1H-inden-2-yl)-5-(phenylsulfonyl)-2-(trifluoromethyl)benzenesulfonamide;
methyl{[2-({[5-(phenylsulfonyl)-2-(trifluoromethyl)phenyl]sulfonyl}amino)-2,3-dihydro-1H-inden-5-yl]oxy}acetate;
{[2-({[5-(phenylsulfonyl)-2-(trifluoromethyl)phenyl]sulfonyl}amino)-2,3-dihydro-1H-inden-5-yl]oxy}acetic acid;
methyl 2-({[5-(phenylsulfonyl)-2-(trifluoromethyl)phenyl]sulfonyl}amino)indane-5-carboxylate;
N-(1-hydroxy-6-methoxy-2,3-dihydro-1H-inden-2-yl)-5-(phenylsulfonyl)-2-(trifluoromethyl)benzenesulfonamide;
2-{[2-({[5-(phenylsulfonyl)-2-(trifluoromethyl)phenyl]sulfonyl}amino)-2,3-dihydro-1H-inden-5-yl]oxy}acetamide;
2-({[5-(phenylsulfonyl)-2-(trifluoromethyl)phenyl]sulfonyl}amino)indane-5-carboxylic acid; or
N-(5-bromo-2,3-dihydro-1H-inden-2-yl)-5-(phenylsulfonyl)-2-(trifluoromethyl)benzenesulfonamide.

49. A pharmaceutical composition comprising at least one compound of claim 1 or a pharmaceutically acceptable salt thereof and one or more pharmaceutically acceptable excipients, diluents, or carriers.

50. A method for treating a patient suffering from osteoporosis, arthritis, chronic obstructive pulmonary disease, cartilage defects, bone fractures, or leiomyoma comprising administering to the patient a therapeutically effective amount of at least one compound of Formula (1): 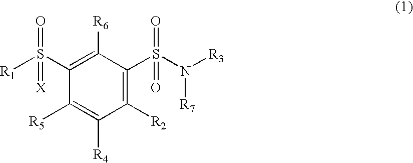
or a pharmaceutically acceptable salt thereof,
wherein
R₁ is 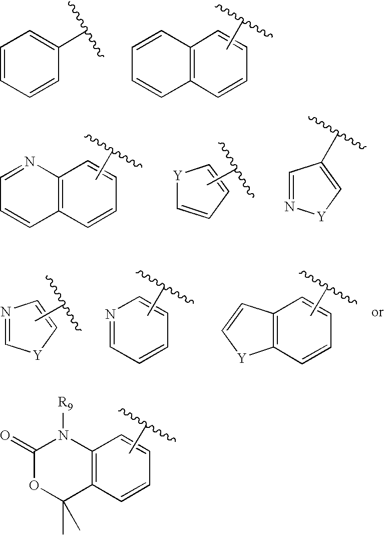
and each R₁ group is optionally substituted with up to three R₈ groups;
Y is O, S, or NR₉;
R₈ is alkyl, arylalkyl, perfluoroalkyl, alkenyl, arylalkenyl, alkynyl, arylalkynyl, cycloalkyl, alkylcycloalkyl, heterocycloalkyl, alkylheterocycloalkyl, aryl, alkylaryl, heteroaryl, alkoxy, perfluoroalkoxy, arylalkoxy, alkylcarbonyl, arylcarbonyl, halogen, cyano, azido, hydroxyl, carboxy, alkoxycarbonyl, alkylamino, dialkylamino, alkylaminocarbonyl, dialkylaminocarbonyl, alkylcarbonylamino, alkylcarbonylalkylamino, hydroxyalkylamino, nitro, alkylcarbonyloxime, alkylsulfonyl, alkylsulfinyl, alkylthio, perfluoroalkylthio, arylthio, tertiary alkylcarbinol, tertiary alkylcycloalkylcarbinol, or tertiary arylalkylcarbinol;
R₉ is hydrogen, alkyl, aryl, arylalkyl, cycloalkylalkyl, heterocycloalkyl, or spirocycloalkyl;
X is oxygen or an electron pair;
R₂ is hydrogen, alkyl, alkoxy, cycloalkyl, perfluoroalkyl, perfluoroalkylalkyl, perfluoroalkoxy, dialkylamino, or halogen;
R₄ is hydrogen, halogen, alkyl, cycloalkyl, alkoxy, perfluoroalkyl, or perfluoroalkoxy;
or R₂ and R₄, together with the carbon atoms to which they are attached, form a cycloalkyl ring of 5 to 7 carbon atoms that is optionally substituted with 1 to 3 R groups;
each R is, independently, hydrogen, alkyl, arylalkyl, cyanoalkyl, cycloalkyl, cycloalkylalkyl, heterocycloalkyl, spirocycloalkyl, aryl, arylalkyl, or alkoxyalkyl;
R₅, and R₆ are, independently, hydrogen, alkyl, aryl, alkoxy, halogen, or perfluoroalkyl;
R₃ and R₇ are each, independently, hydrogen or an optionally substituted alkyl, cycloalkyl, heterocycloalkyl, alkylheterocycloalkyl, heteroarylalkyl, alkylaryl, alkylheteroaryl, alkenyl, alkynyl, fused cycloalkylaryl, fused heterocycloalkylaryl, cycloalkylcarbonyl, or heterocycloalkylcarbonyl group;
or R₃ and R₇, together with the nitrogen atom to which they are attached, form a five or six membered heterocycloalkyl ring optionally substituted with 1 to 5 substituents selected from alkyl, aryl, heterocycloalkyl, heterocycloalkylalkyl, alkylamino, dialkylamino, alkoxycarbonyl, alkylcarbonyl, alkylaminocarbonylalkyl, and heterocycloalkylcarbonylalkyl.

51. The method of claim 50 wherein the patient suffers from osteoporosis or arthritis.
